(19) United States
(12) Patent Application Publication
(10) Pub. No.: US 2024/0228463 A1
(43) Pub. Date: Jul. 11, 2024

(54) SODIUM CHANNEL INHIBITORS AND METHODS OF DESIGNING SAME

(71) Applicant: Genentech, Inc., South San Francisco, CA (US)

(72) Inventors: Marc KSCHONSAK, South San Francisco, CA (US); Steven John MCKERRALL, San Francisco, CA (US); Daniel Fred ORTWINE, Williamsburg, MI (US); Jian Mehr-Dean PAYANDEH, Alameda, CA (US); Benjamin Douglas SELLERS, Larkspur, CA (US); John C. TELLIS, San Francisco, CA (US); Matthew VOLGRAF, Oakland, CA (US); Philippe BERGERON, Redwood City, CA (US); Claudio CIFERRI, Oakland, CA (US); Peter Scott DRAGOVICH, San Diego, CA (US)

(73) Assignee: Genentech, Inc., South San Francisco, CA (US)

(21) Appl. No.: 18/584,311

(22) Filed: Feb. 22, 2024

Related U.S. Application Data
(63) Continuation of application No. PCT/US2022/041258, filed on Aug. 23, 2022.
(60) Provisional application No. 63/236,594, filed on Aug. 24, 2021.

(51) Int. Cl. C07D 401/12; A61K 31/18; A61P 25/04; C07C 311/51; C07D 211/96; C07D 277/44; C07D 277/82; C07D 295/26; C07D 471/04; C07D 487/04; C07D 498/04

(52) U.S. Cl. CPC C07D 401/12; A61K 31/18; A61P 25/04; C07C 311/51; C07D 211/96; C07D 277/44; C07D 277/82; C07D 295/26; C07D 471/04; C07D 487/04; C07D 498/04

(57) ABSTRACT

The invention provides compounds of formulae I, II, IIa, and III:

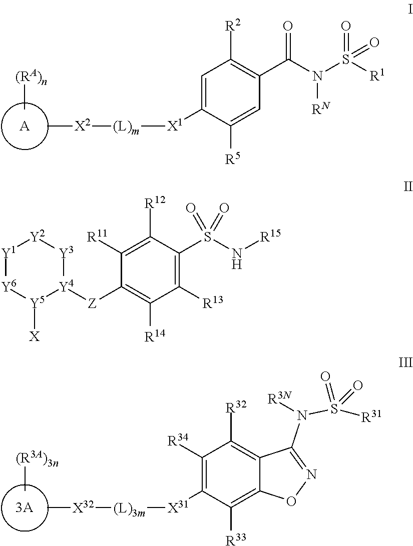

and pharmaceutically acceptable salts thereof, as well as compositions containing such compounds and methods for using such compounds and compositions.

### CLAIM OF PRIORITY

This application is a continuation of International Application Serial No. PCT/US2022/041258, filed Aug. 23, 2022, which application claims the benefit of priority under 35 U.S.C. § 119(e) to U.S. provisional application Ser. No. 63/236,594, filed on Aug. 24, 2021, both of which are incorporated herein by reference.

### BACKGROUND

The present invention relates to organic compounds useful for therapy in a mammal, particularly a human, and in particular to inhibitors of sodium channel (e.g., NaV1.7) that are useful for treating sodium channel-mediated diseases or conditions, such as pain, as well as other diseases and conditions associated with the modulation of sodium channels. The invention further includes methods of designing organic compounds that inhibit the NaV1.7 channel based on atom-resolution structures thereof, such as obtained by cryogenic electron microscopy (“Cryo-EM”, or “cryoEM”).

Voltage-gated sodium channels are transmembrane proteins that initiate action potentials in nerve, muscle and other electrically excitable cells, and are a necessary component of normal sensation, emotions, thoughts and movements (Catterall, W. A., Nature (2001), 409, 988-990). These channels contain a highly processed alpha subunit associated with auxiliary beta subunits. The pore-forming alpha subunit is sufficient for channel function, but the kinetics and voltage dependence of channel gating are in part modified by the beta subunits (Goldin et al, Neuron (2000), 28, 365-368). Electrophysiological recording, biochemical purification, and molecular cloning have identified ten different sodium channel alpha subunits and four beta subunits (Yu, F. H., et al, Sci. STKE (2004), 253: Yu, F. H., et al, Neurosci. (2003), 20:7577-85).

The sodium channel family of proteins has been extensively studied and shown to be involved in a number of vital body functions. Research in this area has identified variants of the alpha subunits that result in major changes in channel function and activities, which can ultimately lead to major pathophysiological conditions. The members of this family of proteins are denoted NaV1.1 to NaV1.9. However, until now, crystal structures of the binding site of sufficient resolution to permit study and design of inhibitor molecules were not available.

NaV1.7 is a tetrodotoxin-sensitive voltage-gated sodium channel encoded by the gene SCN9A. Human NaV1.7 was first cloned from neuroendocrine cells (Klugbauer, N., et al. 1995 EMBO J., 14 (6): 1084-90) and rat NaV1.7 was cloned from a pheochromocytoma PC12 cell line (Toledo-Aral, J. J., et al., Proc. Natl. Acad. Sci. USA (1997), 94:1527-1532) and from rat dorsal root ganglia (Sangameswaran, L., et al., (1997), J. Biol. Chem., 272 (23): 14805-9). NaV1.7 is expressed primarily in the peripheral nervous system, especially nocieptors and olfactory neurons and sympathetic neurons. The inhibition, or blocking, of NaV1.7 has been shown to result in analgesic activity. Knockout of NaV1.7 expression in a subset of sensory neurons that are predominantly nociceptive results in resistance to inflammatory pain (Nassar, et al., op. cit.). Likewise, loss of function mutations in humans results in congenital indifference to pain (CIP), in which the individuals are resistant to both inflammatory and neuropathic pain (Cox, J. J., et al., Nature (2006): 444:894-898; Goldberg, Y. P., et al., Clin. Genet. (2007): 71:311-319). Conversely, gain of function mutations in NaV1.7 have been established in two human heritable pain conditions, primary erythromelalgia and familial rectal pain, (Yang. Y., et al., J. Med. Genet. (2004), 41(3): 171-4). In addition, a single nucleotide polymorphism (R1150W) that has very subtle effects on the time- and voltage-dependence of channel gating has large effects on pain perception (Estacion, M., et al., 2009. Ann Neurol. 66: 862-6; Reimann, F., et al., Proc. Natl. Acad Sci USA (2010), 107: 5148-53). About 10% of the patients with a variety of pain conditions have the allele conferring greater sensitivity to pain and thus might be more likely to respond to block of NaV1.7. Because NaV1.7 is expressed in both sensory and sympathetic neurons, one might expect that enhanced pain perception would be accompanied by cardiovascular abnormalities such as hypertension, but no correlation has been reported. Thus, both the CIP mutations and SNP analysis suggest that human pain responses are more sensitive to changes in NaV1.7 currents than are perturbations of autonomic function.

Inhibitors of NaV1.7 can act through several mechanisms of action: pore binding such as via a local anesthetic, e.g., TTX, STX: peptide voltage sensor domain (VSD) binders such as peptide toxins; and small molecule VSD4 binders, such as aryl- and acylsulfonamides as have previously been identified. Efforts to develop isoform selective NaV1.7 inhibitors have largely focused on VSD4 domain binders.

Sodium channel blockers have been shown to be useful in the treatment of pain, (see. e.g., Wood, J. N., et al, J. Neurobiol. (2004), 61(1), 55-71. Genetic and functional studies have provided evidence to support that activity of NaV1.7 as a major contributor to pain signalling in mammals. (See Hajj, et al. Nature Reviews Neuroscience; 2013, vol 14, 49-62; and Lee, et al. Cell, 2014, vol 157: 1-12). Presently, there are a limited number of effective sodium channel blockers for the treatment of pain with a minimum of adverse side effects which are currently in the clinic. Thus there remains a need for selective voltage-gated sodium channel modulators (e.g., modulators of NaV1.7) that are useful for the treatment of pain. However, efforts to improve upon existing chemical matter via structure-based drug design have been complicated by the recalcitrance of the channel toward X-ray crystallographic co-structure determination.

### SUMMARY

In one aspect the present invention provides novel compounds having sodium channel blocking activity that are useful for the treatment of pain.

In a first embodiment (Embodiment 1; abbreviated as “E1”) the invention provides a compound of Formula I;

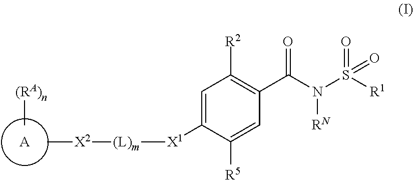

and pharmaceutically acceptable salts thereof, wherein in Formula I, the variables have the following values.

R¹ is selected from a first set of moieties consisting of C₁₋₈alkyl, C₃₋₁₂cycloalkyl, C-linked C₂₋₁₁heterocycloalkyl, C₃₋₁₂carbocycle, aryl, heteroaryl, and —NR^{1A}R^{1B}, wherein;
R^{1A} and R^{1B} are selected from H, C₁₋₈alkyl, C₃₋₈cycloalkyl, C₁₋₈haloalkyl, or R^{1A} and R^{1B} together with the nitrogen atom to which they are both bonded form a 3 to 10 membered heterocyclic group;
any of the first set of moieties, R^{1A}, or R^{1B}, is optionally substituted with one or more substituents selected from a second set of moieties consisting of:
   C₁₋₈alkyl, C₂₋₈alkenyl, C₃₋₈cycloalkyl, C₁₋₈haloalkyl, aryl, (aryl)C₁₋₈alkyl, heteroaryl, (heteroaryl)C₁₋₈alkyl, C₂₋₁₁heterocycloalkyl, (C₂₋₁₁heterocycloalkyl)C₁₋₈alkyl, F, Cl, Br, I, —OH, —CN, —NO₂, ═O, aminocarbonyl, —X^{R1}NR^{R1a}R^{R1b},
   —X^{R1}OR^{R1a}, and —X^{R1}SR^{R1a}, wherein;
      X^{R1} is selected from the group consisting of C(═O), C₁₋₄ alkylene,
      C₁₋₄heteroalkylene, C₂₋₄alkenylene and C₂₋₄ alkynylene, or is absent; and
      R^{R1a} and R^{R1b} are independently selected from a third set of moieties consisting of: H, C₁₋₈alkyl, C₂₋₈alkenyl, C₁₋₈haloalkyl, C₃₋₈cycloalkyl, C₃₋₁₂carbocycle, (C₃₋₈ cycloalkyl)C₁₋₈ alkyl, aryl, (aryl)C₁₋₈alkyl, heteroaryl, (heteroaryl)C₁₋₈alkyl, C₂₋₁₁heterocycloalkyl, and
      (C₂₋₁₁heterocycloalkyl)C₁₋₈alkyl,
      or R^{R1a} and R^{R1b} together with the nitrogen atom to which they are both bonded form a 3 to 10 membered heterocyclic group;
and
wherein any of the second set of moieties, R^{R1a}, or R^{R1b}, where present, are each optionally substituted by one or more groups independently selected from: C₁₋₈ alkyl, C₁₋₈haloalkyl, F, Cl, Br, I, —OH, —CN, aryl, C₁₋₈haloalkyl-substituted aryl, C₁₋₈alkoxy, C₁₋₈alkanoyl, C₁₋₈alkoxycarbonyl, C₃₋₈cycloalkyl, C₂₋₁₁heterocycloalkyl, amino, (C₁₋₃alkyl)amino, di(C₁₋₃alkyl)amino, C₁₋₃alkylamido, C₁₋₃alkylcarboxy, and —NO₂;
R^{N} is hydrogen, C₁₋₄ alkyl, or C₁₋₄haloalkyl;
R² is selected from the group consisting of H, F, Cl, Br, I, —CN, C₁₋₈ alkyl, C₁₋₈ haloalkyl and C₁₋₈ alkoxy;
R⁵ is selected from the group consisting of H, F, Cl, Br, I, —CN, C₁₋₈ alkyl, C₂₋₈ alkenyl, C₂₋₈ alkynyl, C₁₋₈ haloalkyl, C₁₋₈ alkoxy, C₃₋₈ cycloalkyl, C₂₋₁₁ heterocycloalkyl, phenyl and 5-6 membered heteroaryl comprising 1 to 3 heteroatoms selected from N, O and S, wherein said 5-6 membered heteroaryl, C₁₋₈ alkyl, C₃₋₈ cycloalkyl or C₂₋₁₁ heterocycloalkyl is further optionally substituted with from 1 to 3 substituents selected from F, Cl, Br, I, —OH, ═O, C₃₋₆ cycloalkyl, —CN, C₁₋₄ alkyl, —C₁₋₄ alkyl-OC₁₋₄ alkyl, C₁₋₄ haloalkyl and C₁₋₄ alkoxy;
L is a linker selected from the group consisting of C₁₋₄ alkylene, C₂₋₄ alkenylene, C₂₋₄ alkynylene, and C₁₋₄ heteroalkylene, wherein L is optionally substituted with from 1 to 3 substituents selected from the group consisting of —O, —OH, —OCH₂-phenyl, C₁₋₄ alkyl, C₁₋₄ haloalkyl and C₁₋₄ acyl;
m is 0 or 1;
X¹ and X² are each independently selected from the group consisting of, —O—, —S(O)—, —S(O)₂— and —N(R^{X}) wherein R^{X} is H, C₁₋₈ alkyl, C₁₋₈ acyl and —S(O)₂(C₁₋₈ alkyl), or is absent, and wherein if m is 0 then at least one of X¹ or X² is absent;
n is an integer from 0 to 5;
A is selected from the group consisting of: hydrogen, C₁₋₈alkyl, C₁₋₈haloalkyl, C₃₋₁₂ cycloalkyl, and aryl, and wherein if A is hydrogen then n is 0;
each R^{A} is independently selected from the group consisting of C₁₋₈ alkyl, C₃₋₈ cycloalkyl, C₁₋₈ haloalkyl, F, Cl, Br, I, —OH, —CN, —NO₂, ═O, heteroaryl, —(X^{RA})₀₋₁NR^{A1}R^{A2}, —(X^{RA})₀₋₁OR^{A1}, —(X^{RA})₀₋₁SR^{A1}, —(X^{RA})₀₋₁N(R^{A1})C(═O)OR^{A3}, —(X^{RA})₀₋₁OC(═O)N(R^{A1})(R^{A2}), —(X^{RA})₀₋₁N(R^{A1})C(═O)N(R^{A1})(R^{A2}), —(X^{RA})₀₋₁C(═O)N(R^{A1})(R^{A2}), —(X^{RA})₀₋₁N(R^{A1})C(═O)R^{A2}, —(X^{RA})₀₋₁C(═O)OR^{A1}, —(X^{RA})₀₋₁OC(═O)R^{A1}, —P(═O)(OR^{A1})(OR^{A2}), —(X^{RA})₀₋₁S(O)₁₋₂R^{A3}, —(X^{RA})₀₋₁S(O)₁₋₂N(R^{A1})(R^{A2}), —(X^{RA})₀₋₁N(R^{A1})S(O)₁₋₂N(R^{A1})(R^{A2}) and —(X^{RA})₀₋₁N(R^{A1})S(O)₁₋₂(R^{A3}), wherein X^{RA} is selected from the group consisting of C₁₋₄ alkylene, C₁₋₄ heteroalkylene, C₂₋₄ alkenylene and C₂₋₄ alkynylene; wherein R^{A1} and R^{A2} are independently selected from the group consisting of hydrogen, C₁₋₈ alkyl, C₂₋₈ alkenyl, C₁₋₈ haloalkyl, C₃₋₈ cycloalkyl, tetrahydronapthalene, phenyl, benzyl, heteroaryl, and C₂₋₁₁ heterocycloalkyl; R^{A3} is selected from the group consisting of C₁₋₈ alkyl, C₁₋₈ haloalkyl, C₃₋₈ cycloalkyl, tetrahydronapthalene, phenyl, benzyl, heteroaryl, and C₂₋₁₁ heterocycloalkyl; wherein if A is a monocyclic C₃₋₁₂ carbocycloalkyl or monocyclic C₂₋₁₁ heterocycloalkyl, then any two R^{A} substituents attached to adjacent atoms on the A ring are optionally combined to form a benzene or a 5 to 6 membered heteroaryl ring; and wherein the aliphatic and aromatic portions of a R^{A} substitutent is optionally substituted with from 1 to 5 R^{RA} substitutents selected from, F, Cl, Br, I, —NH₂, —OH, —CN, —NO₂, ═O, C₁₋₄ alkyl, C₁₋₄ haloalkyl, C₁₋₄ alkoxy, C₁₋₄ (halo)alkyl-C(═O)—, C₁₋₄ (halo)alkyl-S(O)₀₋₂—, C₁₋₄ (halo)alkyl-C(═O)N(H)—, C₁₋₄ (halo)alkyl-N(H)—C(═O)—, ((halo)alkyl)₂N—C(═O)—, C₁₋₄ (halo)alkyl-OC(═O)N(H)—, C₁₋₄ (halo)alkyl-OC(═O)N(H)—, (halo)alkyl-N(H)—C(═O)O—, ((halo)alkyl)₂N—C(═O)O—, C₁₋₄ alkylamino, C₁₋₄ dialkylamino, C₃₋₆ cycloalkyl, C₃₋₆ cycloalkoxy, C₂₋₈ heterocycloalkoxy, tetrahydronaphthalene and phenyl wherein phenyl is optionally substituted with 1-3 fluoro, chloro, bromo, CN, C₁₋₆ alkyl, C₁₋₆ haloalkyl, C₁₋₆ haloalkoxy, C₁₋₆ alkoxy, C₁₋₆ alkylamino, C₁₋₆ or dialkylamino.

In another embodiment E2, the invention provides a compound or pharmaceutically acceptable salt of E1, wherein n is 0.

In another embodiment E3, the invention provides a compound or pharmaceutically acceptable salt of E1 or E2, wherein A is C₃₋₆cycloalkyl.

In another embodiment E4, the invention provides a compound or pharmaceutically acceptable salt of E1 or E2 wherein A is cyclopentyl.

In another embodiment E5, the invention provides a compound or pharmaceutically acceptable salt of E1, E2, E3, or E4, wherein X¹ is —O—.

In another embodiment E6, the invention provides a compound or pharmaceutically acceptable salt of E1, E2, E3, E4, or E5, wherein X² is absent.

In another embodiment E7, the invention provides a compound or pharmaceutically acceptable salt of E1, E2, E3, E4, E5, or E6, wherein L is C₁₋₄ alkylene.

In another embodiment E8, the invention provides a compound or pharmaceutically acceptable salt of E1, E2, E3, E4, E5, or E6, wherein L is methylene.

In another embodiment E9, the invention provides a compound or pharmaceutically acceptable salt of E1, wherein the group

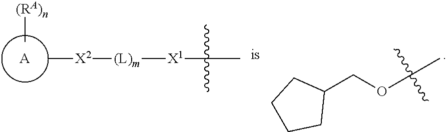

In another embodiment E10, the invention provides a compound or pharmaceutically acceptable salt of E1, E2, E3, E4, E5, E6, E7, E8, or E9, wherein R² is H or F.

In another embodiment E11, the invention provides a compound or pharmaceutically acceptable salt of E1, E2, E3, E4, E5, E6, E7, E8, or E9, wherein R² is F.

In another embodiment E12, the invention provides a compound or pharmaceutically acceptable salt of E1, E2, E3, E4, E5, E6, E7, E8, E9, E10 or E11, wherein R⁵ is selected from the group consisting of H, F, Cl, C₁₋₈ alkyl, C₁₋₈ alkoxy, and C₃₋₈ cycloalkyl.

In another embodiment E13, the invention provides a compound or pharmaceutically acceptable salt of E1, E2, E3, E4, E5, E6, E7, E8, E9, E10 or E11, wherein R⁵ is selected from the group consisting of H, F, Cl, ethyl, isopropyl, cyclopropyl, and methoxy.

In another embodiment E14, the invention provides a compound or pharmaceutically acceptable salt of E1, E2, E3, E4, E5, E6, E7, E8, E9, E10 or E11, wherein R⁵ is selected from the group consisting of methyl, C₁ and cyclopropyl.

In another embodiment E15, the invention provides a compound or pharmaceutically acceptable salt of E1, E2, E3, E4, E5, E6, E7, E8, E9, E10 or E11, wherein R⁵ is Cl.

In another embodiment E16, the invention provides a compound or pharmaceutically acceptable salt of E1, E2, E3, E4, E5, E6, E7, E8, E9, E10, E11, E12, E13, E14, or E15, wherein; R¹ is aryl that is optionally substituted with from 1 or 2 R^{R1} substituents independently selected from the group consisting of C₁₋₃alkyl, trifluoromethyl, C₃₋₅ cycloalkyl, F, Cl, Br, I, —OH, —CN, —(X^{1R})₀₋₁NR^{R1a}R^{R1b}, —(X^{1R})₀₋₁OR^{R1a}, wherein X^{1R} is C₁₋₃ alkylene; and wherein R^{R1a} and R^{R1b} are independently selected from the group consisting of hydrogen, C₁₋₃ alkyl, trifluoromethyl, C₃₋₆ cycloalkyl, phenyl, and benzyl, wherein any phenyl, and benzyl of R^{R1a} and R^{R1b} is optionally substituted with 1, 2, or 3 substituents independently selected from the group consisting of F, Cl, aminomethyl, C₁₋₃ alkyl, C₁₋₃ alkoxy, and dimethylamino;
or R¹ is selected from the group consisting of:

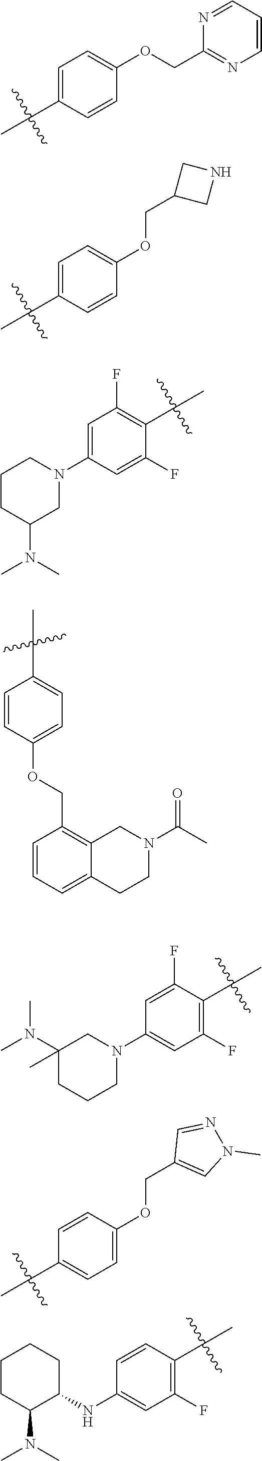

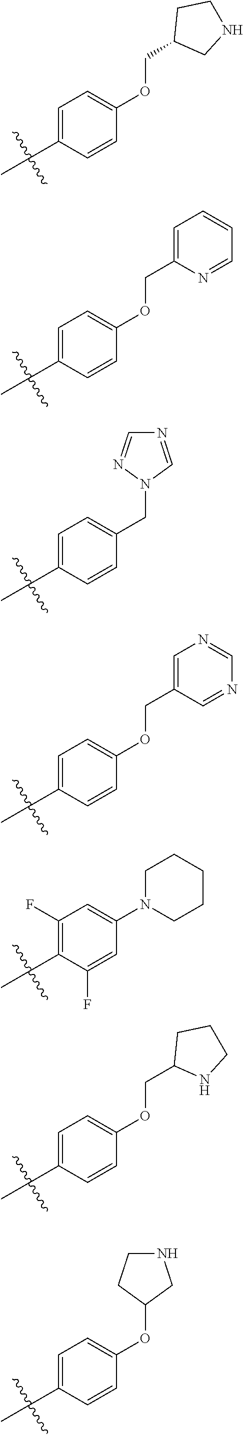

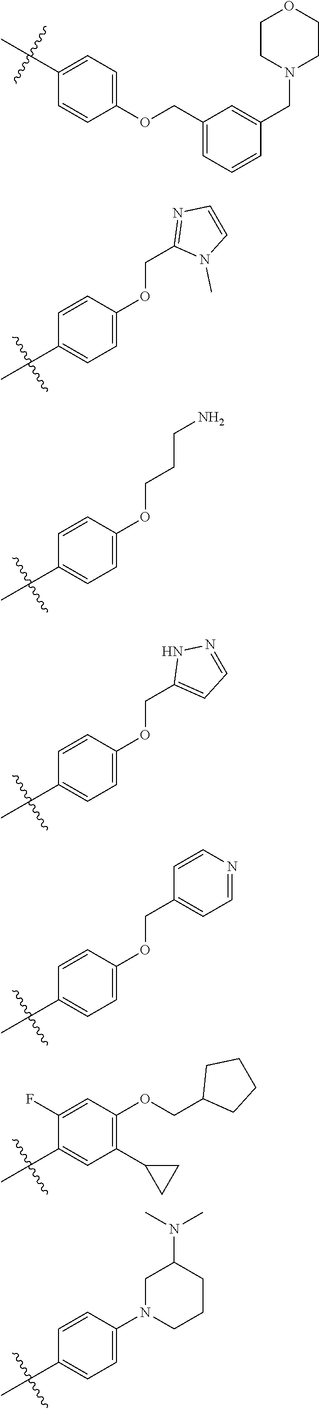

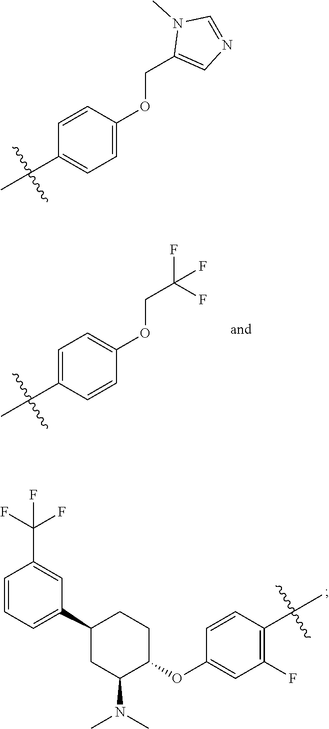 [SMILES: *c1ccc(OCc2cncn2C)cc1 ; *c1ccc(OCC(F)(F)F)cc1 ; *c1ccc(O[C@H]2CC[C@H](c3cccc(C(F)(F)F)c3)C[C@@H]2N(C)C)cc1F]

provided R¹ is not 3-chloro-4-cyanophenyl.

In another embodiment E17, the invention provides a compound or pharmaceutically acceptable salt of E1, E2, E3, E4, E5, E6, E7, E8, E9, E10, E11, E12, E13, E14, or E15, wherein R¹ is selected from the group consisting of any instance of R¹ as found in compounds of the Examples herein, and additionally or including the following groups;

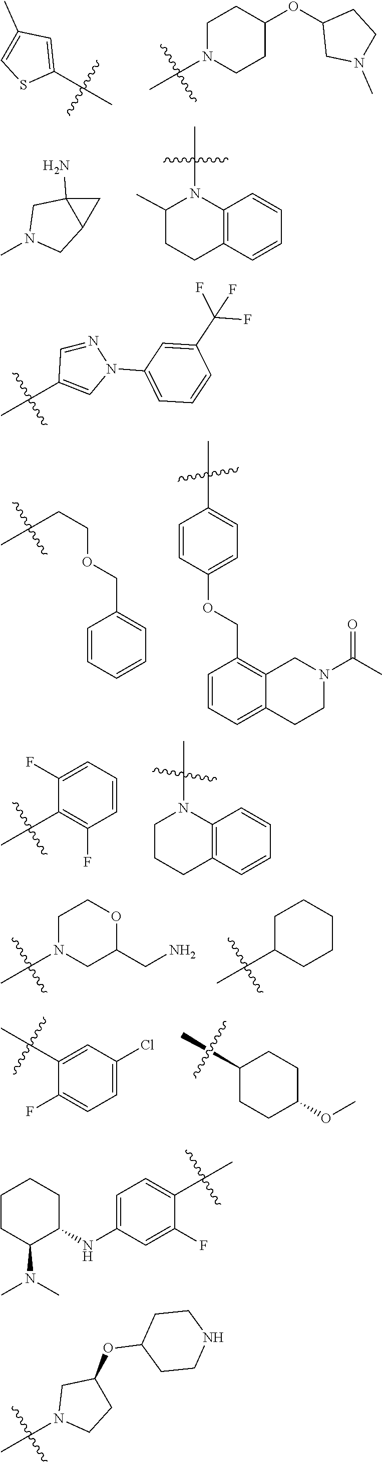

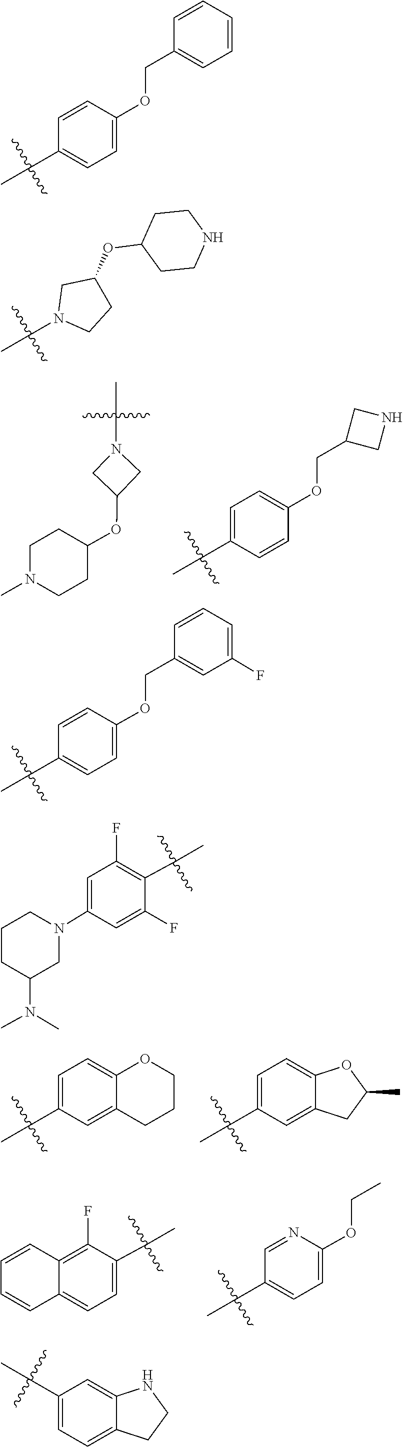

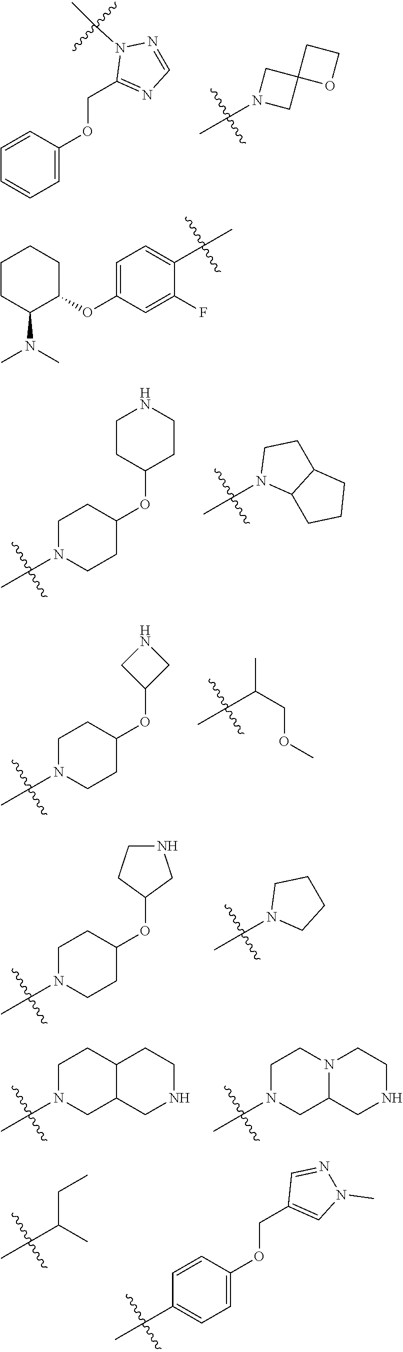

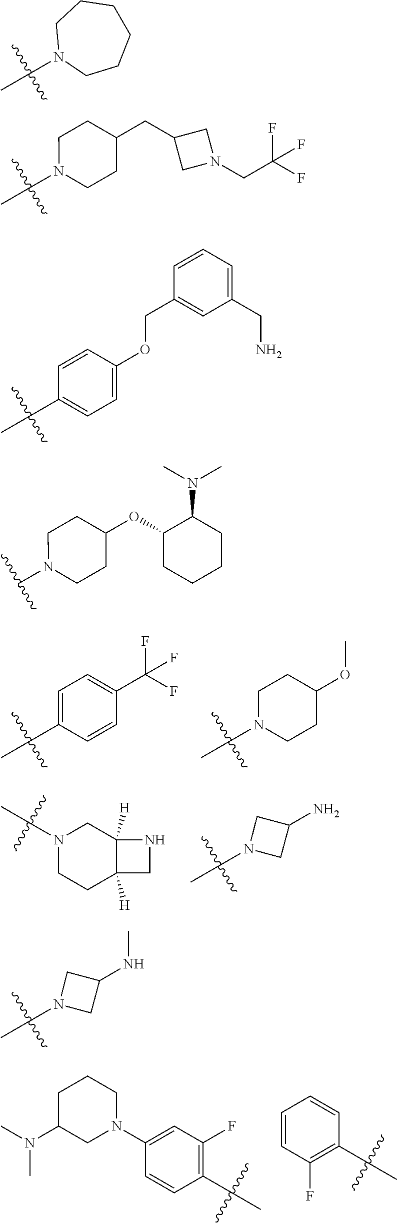

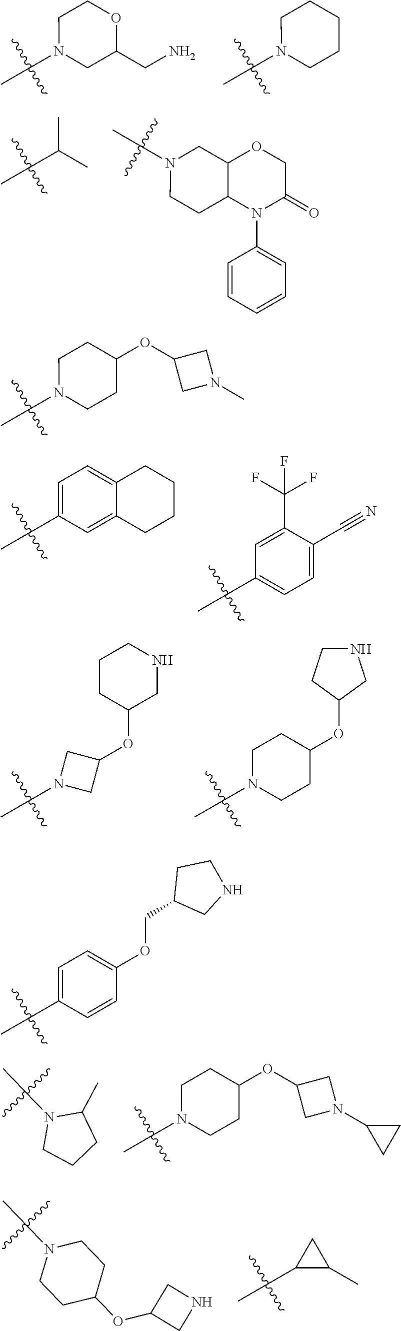

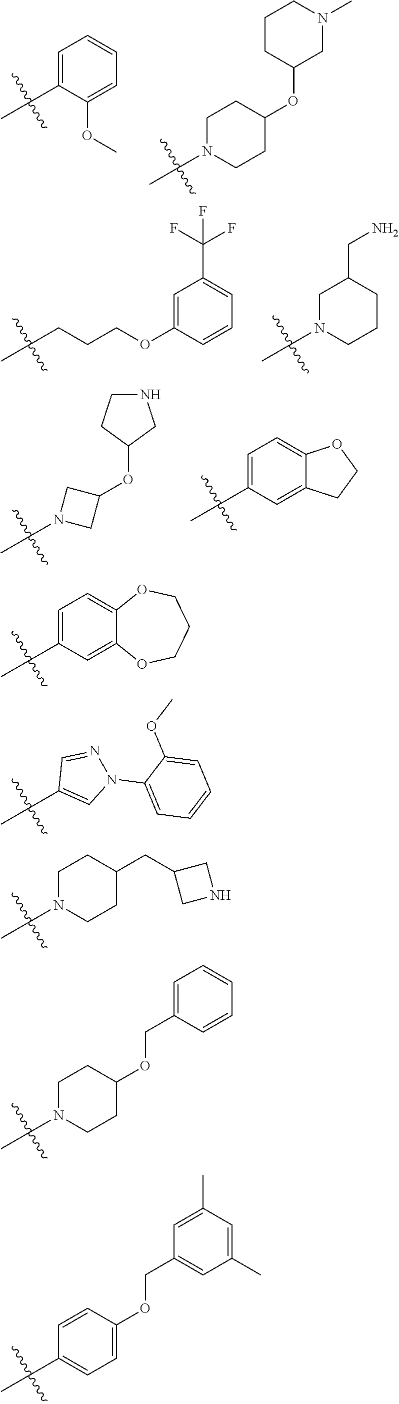

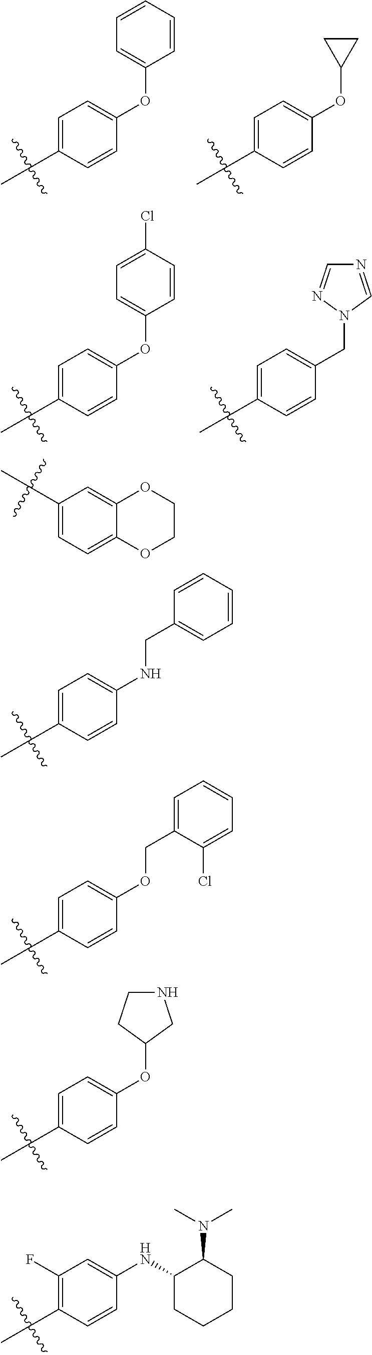

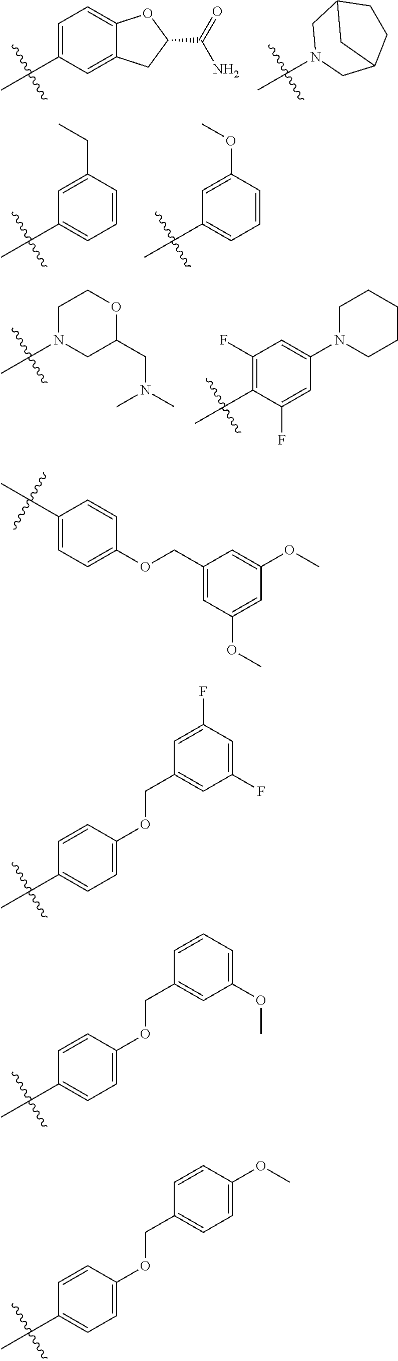

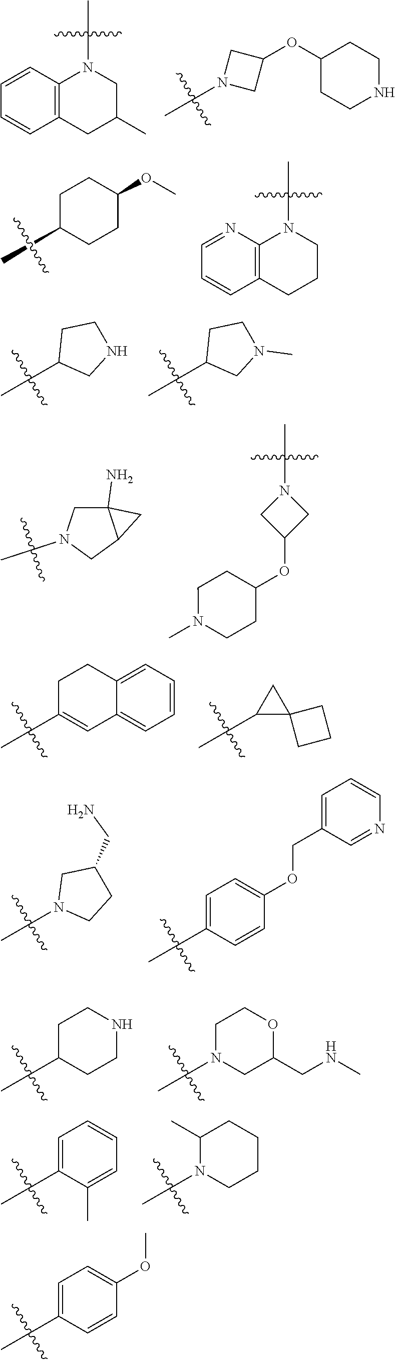

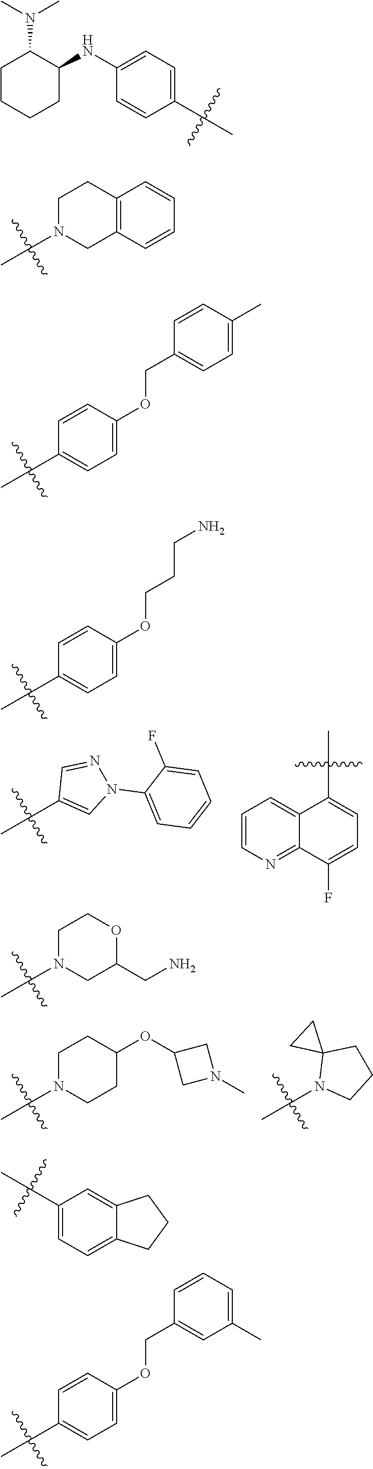

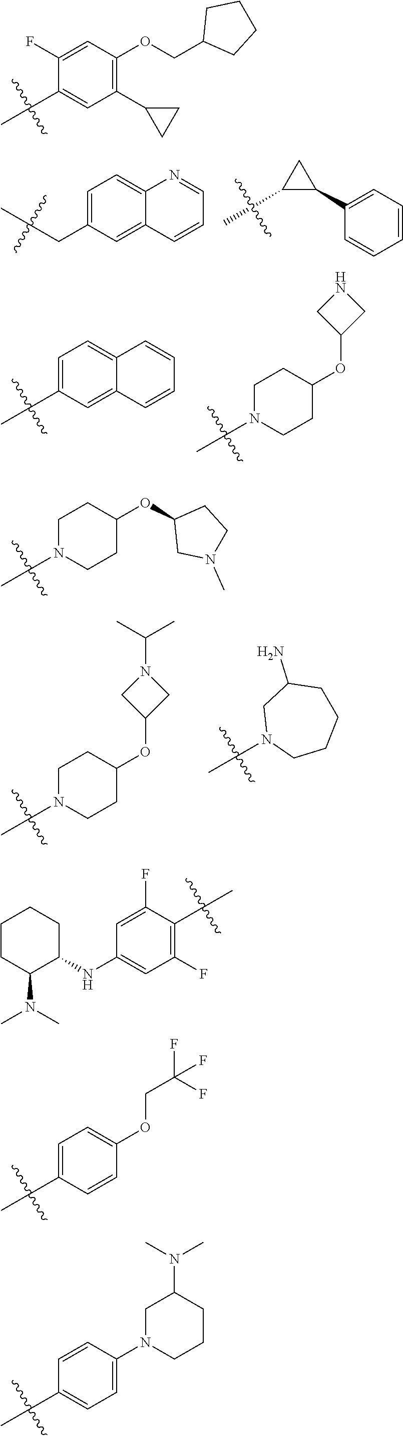

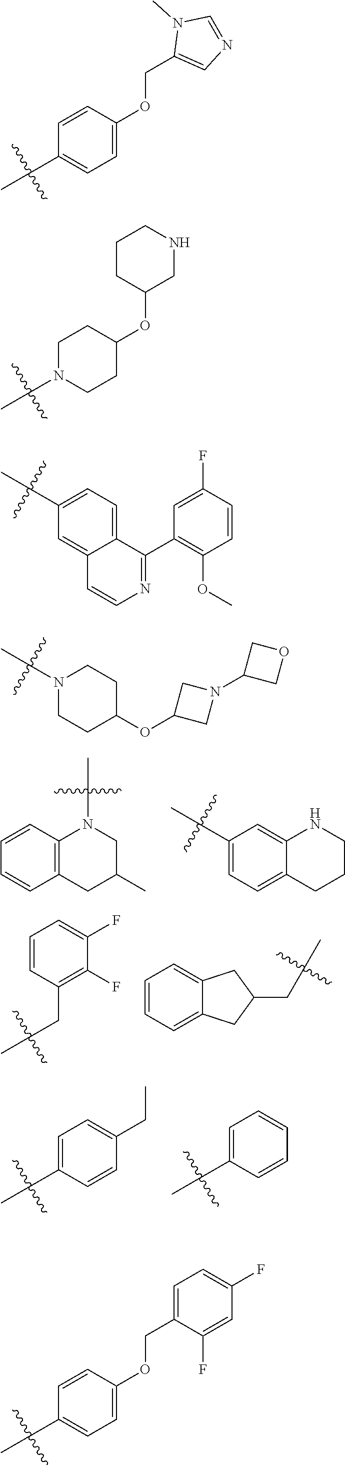

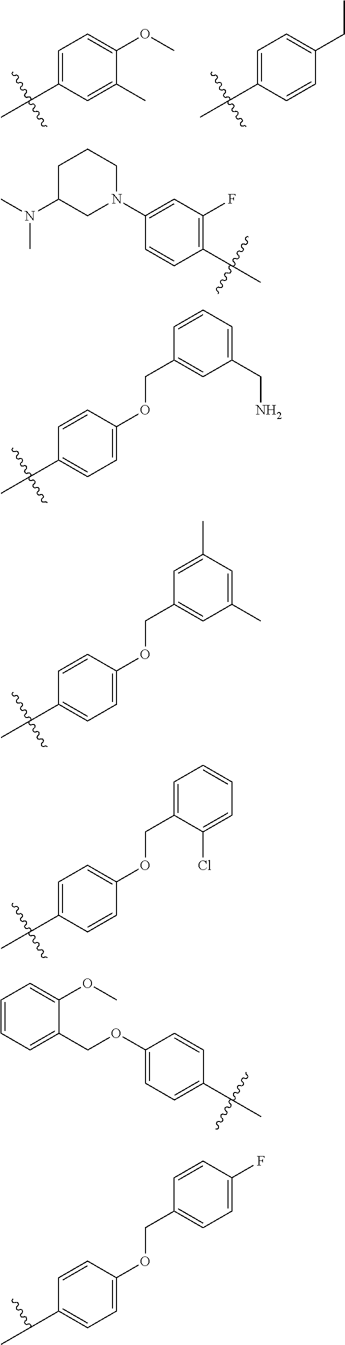

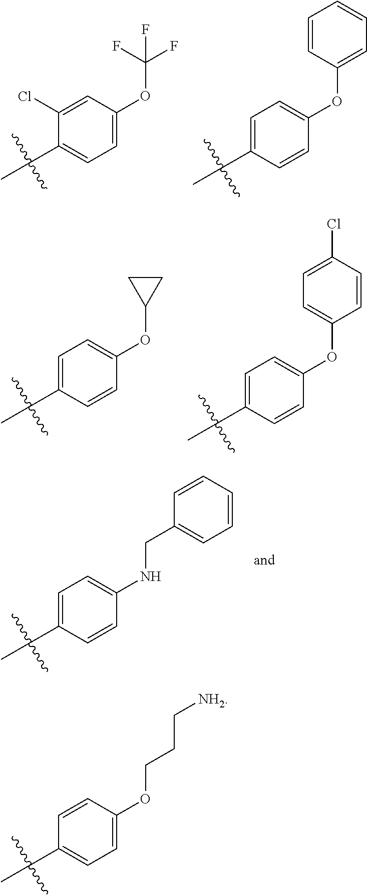 [SMILES: *c1ccc(OC(F)(F)F)cc1Cl ; *c1ccc(Oc2ccccc2)cc1 ; *c1ccc(OC2CC2)cc1 ; *c1ccc(Oc2ccc(Cl)cc2)cc1 ; *c1ccc(NCc2ccccc2)cc1 ; *c1ccc(OCCCN)cc1]

In another embodiment E18, the invention provides a compound or pharmaceutically acceptable salt of E1, E2, E3, E4, E5, E6, E7, E8, E9, E10, E11, E12, E13, E14, E15, E16, or E17 wherein R^{N} is hydrogen.

In another embodiment E19, the invention provides a compound or pharmaceutically acceptable salt of E1, E2, E3, E4, E5, E6, E7, E8, E9, E10, E11, E12, E13, E14, E15, E16, E17, or E18 wherein each R^{A} is independently selected from the group consisting of C₁₋₈ alkyl, C₃₋₈ cycloalkyl, C₁₋₈ haloalkyl, F, Cl, Br, I, —OH, —CN, —NO₂, and ═O.

In another embodiment, E20, the invention provides a compound or pharmaceutically acceptable salt of E1, E2, E3, E4, E5, E6, E7, E8, E9, E10, E11, E12, E13, E14, E15, E16, E17, or E18, wherein each R^{A} is independently selected from the group consisting of C₁₋₈ alkyl, C₃₋₈ cycloalkyl, C₁₋₈ haloalkyl, F, Cl, Br, I, —OH, —CN, —NO₂, and ═O.

In another embodiment E21, the invention provides a compound or pharmaceutically acceptable salt of E1 selected from the group consisting of the compounds described in the individual Examples herein, as well as the following exemplary compounds. Compounds in the following group may also be found individually characterized in the Examples herein. Compounds characterized in the Examples herein that are not specifically enumerated in the following list are also considered to be part of the invention. It is intended that, where specific chirality is not designated at a given chiral center, then either enantiomer or diastereomer of the depicted compound is encompassed.

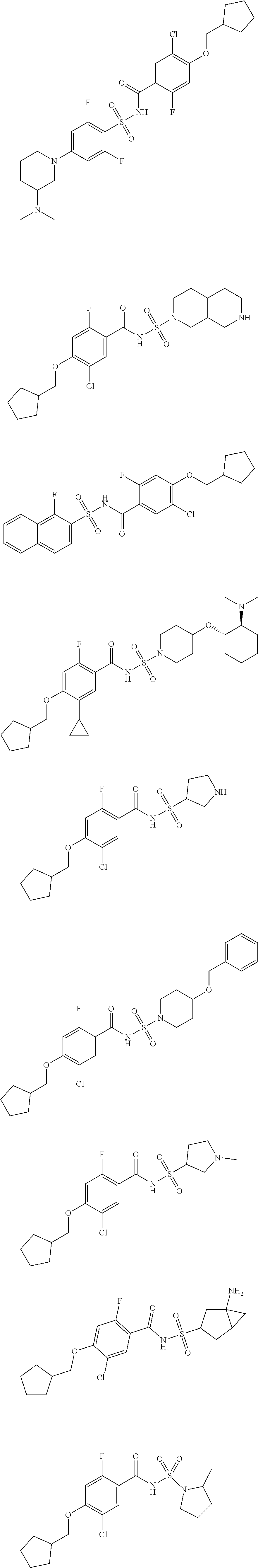

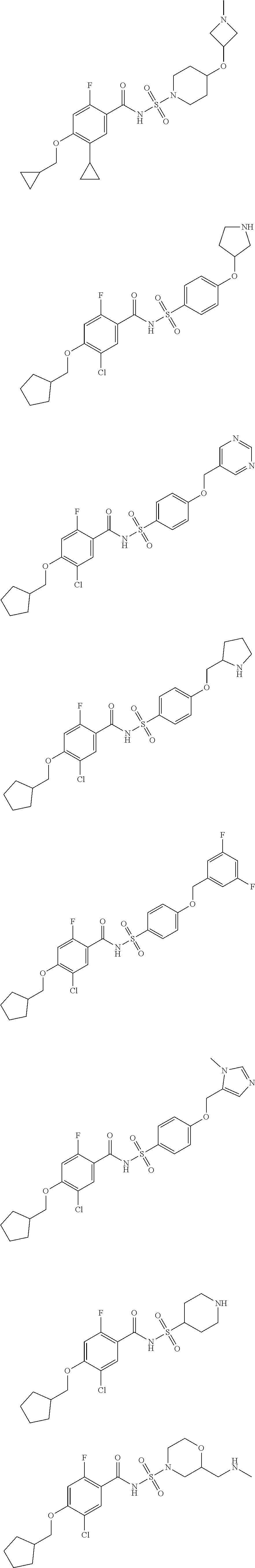

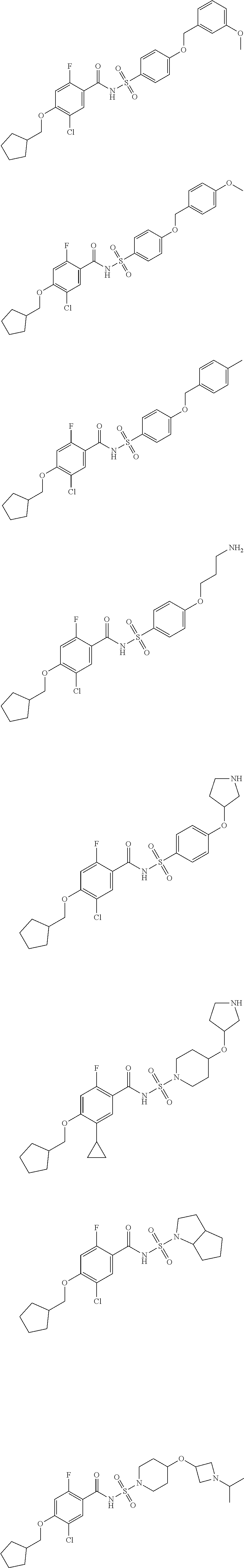

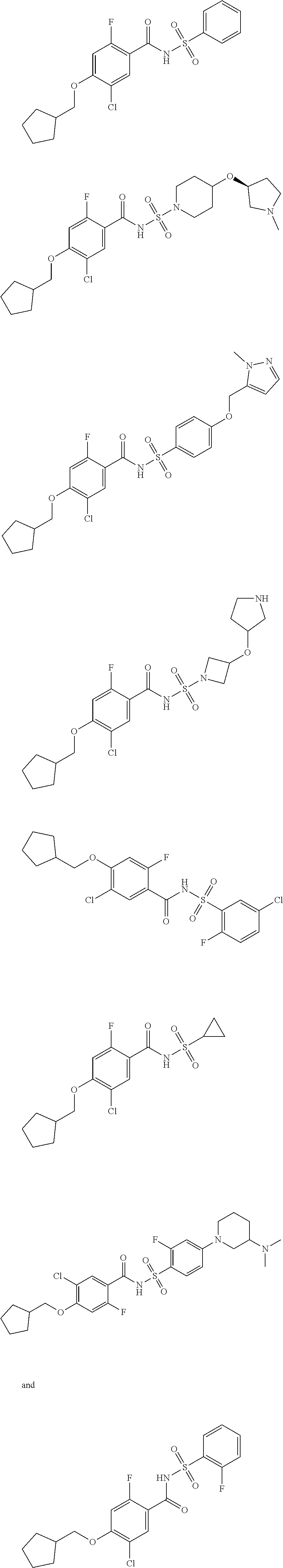

and pharmaceutically acceptable salts thereof.

In another embodiment E22 the invention provides a compound of Formula II or a pharmaceutically acceptable salt thereof, wherein the individual variables are as follows.

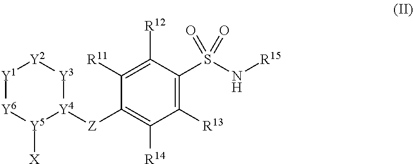

R¹¹, R¹², R¹³, and R¹⁴ are each independently selected from hydrogen, C₁-C₆ alkyl, cyano, C₃-C₆ cycloalkyl, hydroxy, C₁-C₆ alkoxy, —NH₂, —NHR, —NR₂, —SR, —S(O)R, —SO₂R, SO₂NR₂, nitro, and halo, wherein any C₁-C₆ alkyl, and C₃-C₆ cycloalkyl is optionally substituted with one or more groups independently selected from halo, cyano, hydroxy, C₁-C₆alkoxy, C₁-C₆haloalkoxy, C₃-C₆cycloalkyl, and phenyl;
Z is selected from —N(R¹⁵)—, —O—, —S—, —S(O)—, and —S(O)₂—;
R¹⁵ is a 9-membered or 10-membered bicyclic heteroaryl that is optionally substituted with from 1 to 5 R^{R15} substituents selected from the group consisting of C₁₋₈ alkyl, C₂₋₈ alkenyl, C₃₋₈ cycloalkyl, C₁₋₈ haloalkyl, F, Cl, Br, I, —OH, —CN, —NO₂, ═O, —X^{15R}NRR^{15a}R^{R15b}, —X^{15R}OR^{R15a}, —X^{15R}SR^{R15a}, wherein X^{15R} is C₁₋₄ alkylene, or is absent; wherein R^{R15a} and R^{R15b} are independently selected from the group consisting of hydrogen, C₁₋₈ alkyl, C₂₋₈ alkenyl, C₁₋₈ haloalkyl, C₃₋₈ cycloalkyl, (C₃₋₈ cycloalkyl)C₁₋₈ alkyl, aryl, (aryl)C₁₋₈ alkyl, and C₂₋₁₁ heterocycloalkyl; and where any C₁₋₈ alkyl, C₂₋₈ alkenyl, C₃₋₈ cycloalkyl, (C₃₋₈ cycloalkyl)C₁₋₈ alkyl, aryl, (aryl)C₁₋₈ alkyl, and C₂₋₁₁heterocycloalkyl of R^{R15a} and R^{R15b} is optionally substituted with from 1 to 5 substituents independently selected from the group consisting of C₁₋₈ haloalkyl, F, Cl, Br, I, —OH, —CN, and —NO₂;
or R¹⁵ is a 5- or 6-membered heterocycle, that is substituted with one or more —X^{15R}OR^{R15a} or X^{15R}SR^{R15a}, wherein X^{15R} is C₁₋₄ alkylene or is absent; wherein R^{R15a} is hydrogen, C₁₋₈ alkyl, C₂₋₈ alkenyl, C₁₋₈ haloalkyl, C₃₋₈ cycloalkyl,
(C₃₋₈cycloalkyl)C₁₋₈ alkyl, phenyl, (aryl)C₁₋₈ alkyl, and C₂₋₁₁ heterocycloalkyl; where any C₂₋₈ alkenyl, C₃₋₈ cycloalkyl, (C₃₋₈ cycloalkyl)C₁₋₈ alkyl, phenyl, (aryl)C₁₋₈ alkyl, and C₂₋₁₁ heterocycloalkyl of R^{R15a} is optionally substituted with from one or more substituents independently selected from the group consisting of C₁₋₈ haloalkyl, F, Cl, Br, I, —OH, —CN, and —NO₂;
X is selected from hydrogen, C₁₋₈alkyl, —N(R¹⁶)₂ and —N(R¹⁷)₃⁺W⁻, and amine oxides thereof;
W⁻ is a counterion.
Y¹ is —C(R¹⁸)₂—; and Y² is selected from —(C(R¹⁸)₂)ₙ—, —N(R¹⁹)—, —O—, —C(R¹⁸)₂—N(R¹⁹)—, —N(R¹⁹)—C(R¹⁸)₂—, —C(R¹⁸)₂—O—, and —O—C(R¹⁸)₂—; or
Y¹ is selected from —N(R¹⁹)— and —O—; and Y² is —(C(R¹⁸)₂)ₙ—;
n is selected from 0, 1, and 2;
Y³ and Y⁶ are each —C(R¹⁸)₂—;
Y⁴ and Y⁵ are each —C(R¹⁸)—;
each R¹⁶ is independently selected from hydrogen, C₁-C₆ alkyl, C₃-C₆ cycloalkyl, C₁-C₆ alkanoyl, 4-7 membered heterocycle, 5-6 membered heteroaryl, and C₆-C₁₂ aryl, wherein any C₁-C₆ alkyl, C₃-C₆ cycloalkyl, C₁-C₆ alkanoyl, 4-7 membered heterocycle, 5-6 membered heteroaryl, and C₆-C₁₂ aryl is optionally substituted with one or more groups independently selected from deuterium, halo, cyano, hydroxy, C₁-C₆ alkyl, C₁-C₆ alkoxy, C₁-C₆ alkanoyl, C₁-C₆ haloalkyl, C₁-C₆ haloalkoxy, C₃-C₆ cycloalkyl, C₃-C₆ halocycloalkyl, —NR^{a}R^{b}, —C(═O)NR^{a}R^{b}, R^{c}, and phenyl, wherein any C₁-C₆ alkyl, C₁-C₆ alkoxy, and C₁-C₆ alkanoyl is optionally substituted with C₃-C₆ cycloalkyl; or two R¹⁶ groups together with the nitrogen to which they are both attached form a 4- to 10-membered heterocycle that is optionally substituted with one or more groups independently selected from deuterium, halo, cyano, hydroxy, C₁-C₆ alkyl, and C₃-C₆ cycloalkyl, which C₁-C₆ alkyl, and C₃-C₆ cycloalkyl is optionally substituted with one or more groups independently selected from hydroxy and halo;
each R¹⁷ is independently selected from C₁-C₆ alkyl; or two R¹⁷ groups together with the nitrogen to which they are both attached form a 4- to 7-membered heterocycle;
each R¹⁸ is independently selected from hydrogen, C₁-C₆ alkyl, C₁-C₆ haloalkyl, C₃-C₆ cycloalkyl, benzyl, 5-15 membered heteroaryl, C₁-C₆ alkoxy, C₁-C₆ haloalkoxy, C₃-C₆ cycloalkoxy, hydroxy, halo, cyano, and -L-C₆-C₁₂ aryl; wherein each C₃-C₆ cycloalkyl, -L-C₆-C₁₂ aryl, benzyl, 5-15 membered heteroaryl, and C₁-C₆ alkoxy, is optionally substituted with one to three substituents R^{x} each independently selected from C₁-C₆ alkyl, C₃-C₆ cycloalkyl, C₁-C₆ alkoxy, C₁-C₆ haloalkoxy, cyano, halo, hydroxy, C₃-C₆ cycloalkoxy, C₃-C₆ halocycloalkyl, —S(C₁-C₆ alkyl), —S(O)(C₁-C₆ alkyl), —S(O)₂(C₁-C₆ alkyl), —NH₂, —NH(C₁-C₆ alkyl), —N(C₁-C₆ alkyl)₂, 4-6 membered heterocycle, and C₁-C₆ haloalkyl or two R¹⁸ that are on adjacent carbons taken together form a double bond; and R¹⁹ is selected from hydrogen, C₁-C₆ alkyl and C₆-C₁₂ aryl, which C₆-C₁₂ aryl is optionally substituted with one to three substituents each independently selected from C₁-C₆ alkyl, C₃-C₆ cycloalkyl, C₁-C₆ alkoxy, C₁-C₆ haloalkoxy, cyano, halo, and C₁-C₆ haloalkyl; or one R¹⁸ or R¹⁹ taken together with another R¹⁸ or R¹⁹ and the atoms to which they are attached form a 3-8 membered fused, bridged or spirocyclic ring, which 3-8 membered ring is optionally substituted with one to three substituents each independently selected from C₁—C₆ alkyl, C₁-C₆ alkoxy, cyano, halo, and C₁-C₆ haloalkyl; and L is selected from a bond, —O—, —S—, —S(O)—, and —S(O)₂—;
each R^{a} and R^{b} is independently selected from the group consisting of hydrogen, C₁-C₆ alkyl, and C₁-C₆ alkanoyl;
each R^{c} is independently selected from 4-7 membered heterocycle that is optionally substituted with one or more groups independently selected from halo, C₁-C₆ alkyl, and C₁-C₆ haloalkyl; and

In another embodiment E23, the invention provides a compound or pharmaceutically acceptable salt of embodiment E22, which is a compound of formula IIa, wherein X, Z, R¹¹-R¹⁵ and R¹⁸ are as for formula II;

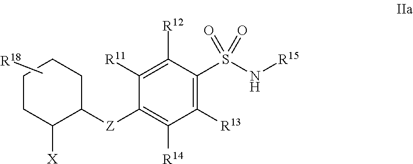

or a pharmaceutically acceptable salt thereof.

In another embodiment E24, the invention provides a compound or pharmaceutically acceptable salt of E22 or E23, wherein R¹⁸ is trifluoromethyl or phenyl that is optionally substituted with one to three substituents each independently selected from C₁-C₆ alkyl, cyano, halo, and C₁-C₆ haloalkyl; or a pharmaceutically acceptable salt thereof.

In another embodiment E25, the invention provides a compound or pharmaceutically acceptable salt of E22, E23 or E24, wherein X is —N(R¹⁶)₂ and each R¹⁶ is independently selected from hydrogen, C₂-C₆ alkoxyalkyl, C₁-C₆ alkyl, benzyl, C₃-C₆cycloalkyl, C₁-C₆haloalkyl, and C₁-C₆hydroxyalkyl; or a pharmaceutically acceptable salt thereof.

In another embodiment E26, the invention provides a compound or pharmaceutically acceptable salt of E22, E23 or E24 wherein X is —N(R¹⁶)₂ and two R¹⁶ groups together with the nitrogen to which they are both attached form a 4- to 7-membered heterocycle, or a pharmaceutically acceptable salt thereof.

In another embodiment E27, the invention provides a compound or pharmaceutically acceptable salt of E22, E23, or E24, wherein X is dimethylamino, or a pharmaceutically acceptable salt thereof.

In another embodiment E28, the invention provides a compound or pharmaceutically acceptable salt of E22, E23, E24, E25, E26, or E27, wherein any of R¹¹, R¹², R¹³, or R¹⁴ is selected from fluoro, chloro, bromo, cyclopropyl, and trifluoromethyl; or a pharmaceutically acceptable salt thereof.

In another embodiment E29, the invention provides a compound or pharmaceutically acceptable salt of E22, E23, E24, E25, E26, E27, or E28, wherein R¹¹ is H, or a pharmaceutically acceptable salt thereof.

In another embodiment E30, the invention provides a compound or pharmaceutically acceptable salt of E22, E23, E24, E25, E26, E27, or E28, wherein R¹¹ is selected from hydrogen and halo; or a pharmaceutically acceptable salt thereof.

In another embodiment E31, the invention provides a compound or pharmaceutically acceptable salt of E22, E23, E24, E25, E26, E27, E28, E29 or E30, wherein R¹³ is selected from hydrogen and fluoro; or a pharmaceutically acceptable salt thereof.

In another embodiment E32, the invention provides a compound or pharmaceutically acceptable salt of E22, E23, E24, E25, E26, E27, E28, E29, E30, or E31 wherein R¹² is hydrogen; or a pharmaceutically acceptable salt thereof.

In another embodiment E33, the invention provides a compound or pharmaceutically acceptable salt of E22, E23, E24, E25, E26, E27, E28, E29, E30, E31, or E32, wherein R¹⁴ is hydrogen; or a pharmaceutically acceptable salt thereof.

In another embodiment E34, the invention provides a compound or pharmaceutically acceptable salt of E22, E23, E24, E25, E26, E27, E29, E30, E31, or E32, wherein R¹¹, R¹², R¹³, or R¹⁴ is selected from hydrogen, fluoro, chloro, bromo, cyclopropyl, cyclobutyl, hydroxy, methoxy, and trifluoromethyl; or a pharmaceutically acceptable salt thereof.

In another embodiment E35, the invention provides a compound or pharmaceutically acceptable salt of E22, E23, E24, E25, E26, E27, E28, E29, E30, E31, E32, E33, or E34, wherein R¹⁵ is a 9-membered or 10-membered bicyclic heteroaryl that is optionally substituted with from 1 to 5 R^{R15} substituents selected from the group consisting of C₁₋₈ alkyl, C₂₋₈ alkenyl, C₃₋₈ cycloalkyl, C₁₋₈ haloalkyl, F, Cl, Br, I, —OH, —CN, —NO₂, ═O, —(X^{15R})₀₋₁NRR^{15a}R^{R15b}, —(X^{15R})₀₋₁OR^{R15a}, —(X^{15R})₀₋₁SR^{R15a}, wherein X^{15R} is C₁₋₄ alkylene; wherein R^{R15a} and R^{R15b} are independently selected from the group consisting of hydrogen, C₁₋₈ alkyl, C₂₋₈ alkenyl, C₁₋₈ haloalkyl, C₃₋₈ cycloalkyl, (C₃₋₈ cycloalkyl)C₁₋₈ alkyl, aryl, (aryl)C₁₋₈ alkyl, and C₂₋₁₁ heterocycloalkyl; and where any C₂₋₈ alkenyl, C₃₋₈ cycloalkyl, (C₃₋₈ cycloalkyl)C₁₋₈ alkyl, phenyl, (aryl)C₁₋₈ alkyl, and C₂₋₁₁ heterocycloalkyl of R^{R15a} and R^{R15b} is optionally substituted with from 1 to 5 substituents independently selected from the group consisting of C₁₋₈ haloalkyl, F, Cl, Br, I, —OH, —CN, and —NO₂.

In another embodiment E36, the invention provides a compound or pharmaceutically acceptable salt of E22, E23, E24, E25, E26, E27, E28, E29, E30, E31, E32, E33. E34, or E35, wherein R¹⁵ is selected from the group consisting of:

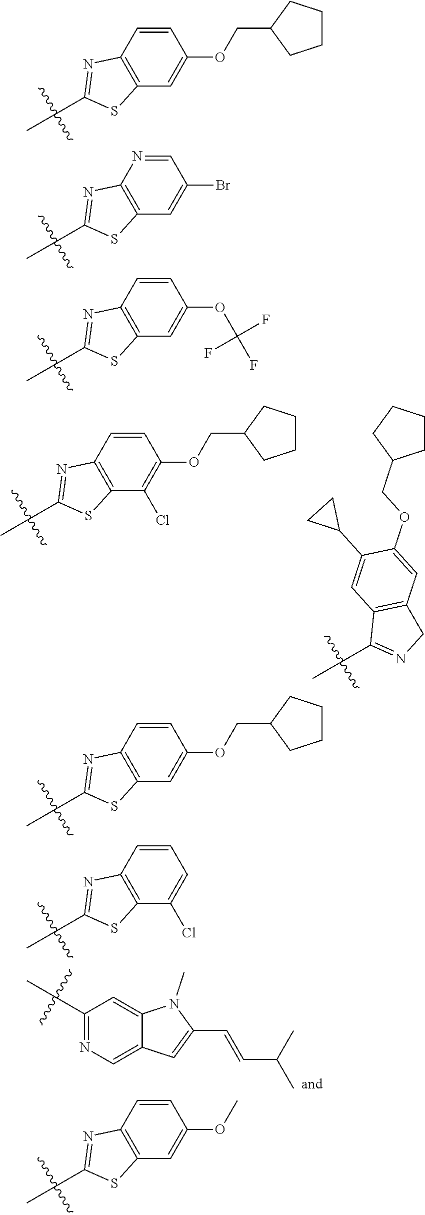

In another embodiment E37, the invention provides a compound or pharmaceutically acceptable salt of E22, E23, E24, E25, E26, E27, E28, E29, E30, E31, E32, E33. E34, or E35, wherein R¹⁵ is a heterocycle selected from thiazole, thiadiazole, oxazole, isoxazole, pyrimidine, pyridazine, and pyridyl, wherein the heterocycle is substituted with one or more —(X^{15R})₀₋₁OR^{R15a} or —(X^{15R})₀₋₁SR^{R15a}, wherein X^{15R} is C₁₋₄ alkylene; wherein R^{R15a} is hydrogen, C₁₋₈ alkyl, C₂₋₈ alkenyl, C₁₋₈ haloalkyl, C₃₋₈ cycloalkyl, (C₃₋₈ cycloalkyl)C₁₋₈ alkyl, phenyl, (aryl)C₁₋₈ alkyl, and C₂₋₁₁ heterocycloalkyl; where any C₂₋₈ alkenyl, C₃₋₈ cycloalkyl, (C₃₋₈ cycloalkyl)C₁₋₈ alkyl, phenyl, (aryl)C₁₋₈ alkyl, and C₂₋₁₁ heterocycloalkyl of R^{R15a} is optionally substituted with from 1 to 5 substituents independently selected from the group consisting of C₁₋₈ haloalkyl, F, Cl, Br, I, —OH, —CN, and —NO₂.

In another embodiment E38, the invention provides a compound or pharmaceutically acceptable salt of E22, E23, E24, E25, E26, E27, E28, E29, E30, E31, E32, E33, or E34, wherein R¹⁵ is selected from the group consisting of:

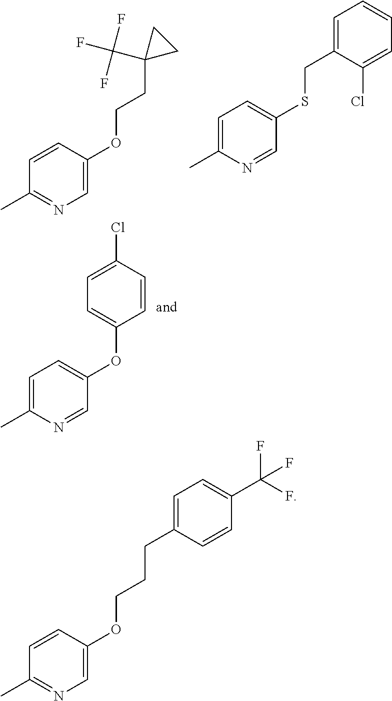

In another embodiment E40, the invention provides a compound or pharmaceutically acceptable salt of E22 selected from the group consisting of the compounds described in the individual Examples herein, including but not limited to the compounds of Examples 89, 95, 96, 97, 99, 101, 102, 103, 201 202, 203, 257, 258, and 333, and pharmaceutically acceptable salts thereof, wherein it is intended that, where specific chirality is not designated at a given chiral center, then either enantiomer or diastereomer of the depicted compound is encompassed.

In another embodiment E41 the invention provides a compound of the invention, which is a compound of Formula III;

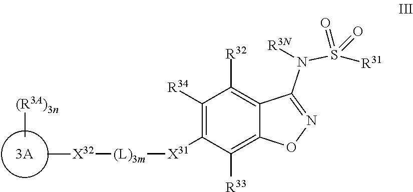

or a pharmaceutically acceptable salt thereof, wherein the individual variables are as follows.

R³¹ is selected from a first set of moieties consisting of C₁₋₈alkyl, C₃₋₁₂cycloalkyl, C-linked C₂₋₁₁heterocycloalkyl, C₃₋₁₂carbocycle, aryl, heteroaryl, and —NR^{31A}R^{31B}, wherein;
R^{31A} and R^{31B} are selected from H, C₁₋₈alkyl, C₃₋₈cycloalkyl, C₁₋₈haloalkyl, or R^{31A} and R^{31B} together with the nitrogen atom to which they are both bonded form a 3 to 10 membered heterocyclic group;
any of the first set of moieties, R^{31A}, or R^{31B}, is optionally substituted with one or more substituents selected from a second set of moieties consisting of:
   C₁₋₈alkyl, C₂₋₈alkenyl, C₃₋₈cycloalkyl, C₁₋₈haloalkyl, aryl, (aryl)C₁₋₈alkyl, heteroaryl, (heteroaryl)C₁₋₈alkyl, C₂₋₁₁heterocycloalkyl,
   (C₂₋₁₁heterocycloalkyl)C₁₋₈alkyl, F, Cl, Br, I, —OH, —CN, —NO₂, ═O, aminocarbonyl, —X^{R1}NR^{R1a}R^{R1b}
   —X^{R1}OR^{R1a}, and —X^{R1}SR^{R1a}, wherein;
      X^{R1} is selected from the group consisting of C(—O), C₁₋₄ alkylene,
      C₁₋₄heteroalkylene, C₂₋₄alkenylene and C₂₋₄ alkynylene, or is absent; and
      R^{R1a} and R^{R1b} are independently selected from a third set of moieties consisting of: H, C₁₋₈alkyl, C₂₋₈alkenyl, C₁₋₈haloalkyl, C₃₋₈cycloalkyl,
      C₃₋₁₂carbocycle, (C₃₋₈ cycloalkyl)C₁₋₈ alkyl, aryl, (aryl)C₁₋₈alkyl, heteroaryl, (heteroaryl)C₁₋₈alkyl, C₂₋₁₁heterocycloalkyl, and
      (C₂₋₁₁heterocycloalkyl)C₁₋₈alkyl,
      or R^{R1a} and R^{R1b} together with the nitrogen atom to which they are both bonded form a 3 to 10 membered heterocyclic group;
and
wherein any of the second set of moieties, R^{R1a}, or R^{R1b}, where present, are each optionally substituted by one or more groups independently selected from: C₁₋₈ alkyl, C₁₋₈haloalkyl, F, Cl, Br, I, —OH, —CN, aryl, C₁₋₈haloalkyl-substituted aryl, C₁₋₈alkoxy, C₁₋₈alkanoyl, C₁₋₈alkoxycarbonyl, C₃₋₈cycloalkyl, C₂₋₁₁heterocycloalkyl, amino, (C₁₋₃alkyl)amino, di(C₁₋₃alkyl)amino, C₁₋₃alkylamido, C₁₋₃alkylcarboxy, and —NO₂;

R^{3N} is hydrogen, C₁₋₄ alkyl or C₁₋₄ haloalkyl.

R³² and R³³ are independently selected from the group consisting of H, F, Cl, Br, I, —CN, C₁₋₈ alkyl, C₁₋₈ haloalkyl and C₁₋₈ alkoxy.

R³⁴ is selected from the group consisting of H, F, Cl, Br, I, —CN, C₁₋₈ alkyl, C₂₋₈ alkenyl, C₁₋₈ haloalkyl, C₁₋₈ alkoxy, C₃₋₈ cycloalkyl, C₂₋₁₁ heterocycloalkyl, phenyl and 5-6 membered heteroaryl comprising 1 to 3 heteroatoms selected from N, O and S, wherein said 5-6 membered heteroaryl, C₁₋₈ alkyl, C₃₋₈ cycloalkyl or C₂₋₁₁ heterocycloalkyl is further optionally substituted with from 1 to 3 R^{5a} substituents selected from F, Cl, Br, I, —OH, —O, C₃₋₆ cycloalkyl, —CN, C₁₋₄ alkyl, —C₁₋₄ alkyl-O—C₁₋₄ alkyl, C₁₋₄ haloalkyl and C₁₋₄ alkoxy.

L is a linker selected from the group consisting of C₁₋₄ alkylene, C₂₋₄ alkenylene, C₂₋₄ alkynylene, and C₁₋₄ heteroalkylene, wherein L is optionally substituted with from 1 to 3 substituents selected from the group consisting of —O, —OH, —OCH₂-phenyl, C₁₋₄ alkyl, C₁₋₄ haloalkyl and C₁₋₄ acyl.

3m is 0 or 1.

3n is an integer from 0 to 5.

X³¹ and X³² are each independently selected from the group consisting of —O—, —S(O)—, —S(O)₂— and —N(R^{X})— wherein R^{X} is H, C₁₋₈ alkyl, C₁₋₈ acyl or —S(O)₂(C₁₋₈ alkyl), or is absent, and wherein if 3m is 0 then at least one of X³¹ or X³² is absent.

3A is selected from the group consisting of hydrogen, C₁₋₈alkyl, C₁₋₈haloalkyl, C₃₋₁₂ cycloalkyl, and aryl, and wherein if 3A is hydrogen then n is 0.
each R^{3A} is independently selected from the group consisting of C₁₋₈ alkyl, C₃₋₈ cycloalkyl, C₁₋₈ haloalkyl, F, Cl, Br, I, —OH, —CN, —NO₂, and ═O.

In another embodiment E42, the invention provides a compound or pharmaceutically acceptable salt of E41, wherein X³¹ is —O—.

In another embodiment E43, the invention provides a compound or pharmaceutically acceptable salt of E41 or E42, wherein X³² is absent.

In another embodiment E44, the invention provides a compound or pharmaceutically acceptable salt of E41, E42, or E43, wherein L is C₁₋₄ alkylene.

In another embodiment E45, the invention provides a compound or pharmaceutically acceptable salt of E41, E42, or E43, wherein L is methylene.

In another embodiment E46, the invention provides a compound or pharmaceutically acceptable salt of E41, wherein the group

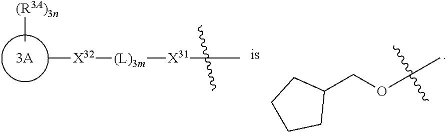

In another embodiment E47, the invention provides a compound or pharmaceutically acceptable salt of E41, E42, E43, E44, E45, or E46, wherein R³² is H.

In another embodiment E48, the invention provides a compound or pharmaceutically acceptable salt of E41, E42, E43, E44, E45, E46, or E47, wherein R³³ is H.

In another embodiment E49, the invention provides a compound or pharmaceutically acceptable salt of E41, E42, E43, E44, E45, E46, E47, or E48, wherein R³⁴ is selected from the group consisting of F, Cl, ethyl, isopropyl, cyclopropyl, and methoxy.

In another embodiment E50, the invention provides a compound or pharmaceutically acceptable salt of E41, E42, E43, E44, E45, E46, E47, or E48, wherein R³⁴ is selected from the group consisting of C₁ and cyclopropyl.

In another embodiment E51, the invention provides a compound or pharmaceutically acceptable salt of E41, E42, E43, E44, E45, E46, E47, E48, E49, or E50, wherein R^{N} is hydrogen.

In another embodiment E52, the invention provides a compound or pharmaceutically acceptable salt of E41, E42, E43, E44, E45, E46, E47, E48, E49, E50, or E51, wherein R³¹ is;

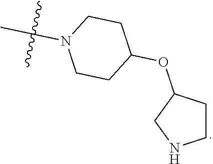 [SMILES: *N1CCC(OC2CCNC2)CC1]

In another embodiment E53, the invention provides a compound or pharmaceutically acceptable salt of E41 that is that is selected from the group consisting of compounds described in the individual Examples herein, including but not limited to the compounds of Examples 89, 90, 91, 93 and 94, and pharmaceutically acceptable salts thereof, wherein it is intended that, where specific chirality is not designated at a given chiral center, then either enantiomer or diastereomer of the depicted compound is encompassed.

In another embodiment E54 the invention provides a compound having the following structure;

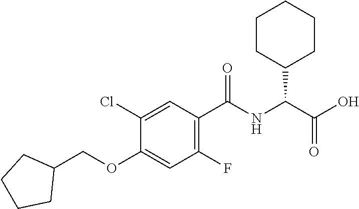 [SMILES: O=C(N[C@@H](C(=O)O)C1CCCCC1)c1cc(Cl)c(OCC2CCCC2)cc1F]

or a pharmaceutically acceptable salt thereof.

In another embodiment E55, the invention provides a compound or pharmaceutically acceptable salt of embodiments E1-E20 of Formula (I), wherein X¹ is —O— or —N(H)—; X² is absent; m is 1; and L is selected from the group consisting of —CH₂—, —C(═O)—, —C(H)(CH₃)—, —CH₂—CH₂—, —CH₂—C(H)(CH₃)—, —C(H)(CH₃)—C(H₂)—, —CH₂CH₂CH₂—, —CH₂—C(H)(CH₃)—CH₂— or —CH₂CH₂CH₂CH₂—.

In another embodiment E56, the invention provides a compound or pharmaceutically acceptable salt of embodiments E1-E20 of Formula (I), wherein A is an optionally substituted ring selected from the group consisting of cyclopropane, cyclobutane, cyclopentane, cyclohexane, cycloheptane, adamantane, bicyclo[2.1.1]hexane, bicyclo[2.2.2]octane, bicyclo[2.2.1]heptane, bicyclo[3.1.1]heptane, bicyclo[3.2.1]octane, bicyclo[4.1.1]octane, bicyclo[3.3.1]nonane and 1,2,3,4-tetrahydro-1,4-methanonaphthalene, 1,2,3,4-tetrahydroisoquinoline and chroman.

In another embodiment E57, the invention provides a compound or pharmaceutically acceptable salt of embodiments E1-E20 of Formula (I), wherein A is an optionally substituted ring selected from the group consisting of cyclopropane, cyclobutane, cyclopentane, cyclohexane, adamantane, cubane, bicyclo[2.2.2]octane, bicyclo[3.1.1]heptane, bicyclo[2.2.1]heptane, piperidinyl, tetrahydrofuranyl, tetrahydronaphthyl, spiro[2,5]octanyl, norpinanyl, spiro[3.5]nonanyl, 8-azabicyclo[3.2.1]octanyl, norbornanyl, spiro[4.5]decanyl, bicyclo[4.1.0]heptane and spiro[5.5]undecanyl.

In another embodiment E58, the invention provides a compound or pharmaceutically acceptable salt of embodiments E1-E20 of Formula (I), wherein A is an optionally substituted ring selected from the group consisting of azetidine, pyrrolidine, piperidine, homopiperidine, (1R,5S)-8-azabicyclo[3.2.1]octane, 3-oxa-9-azabicyclo[3.3.1]nonane, (1s, 4s)-7-azabicyclo[2.2.1]heptane, (1R,4S)-5-azabicyclo[2.1.1]hexane, 7-(trifluoromethyl)-1,2,3,4-tetrahydropyrrolo[1,2-a]pyrazine and quinuclidine.

In another embodiment E59, the invention provides a compound or pharmaceutically acceptable salt of embodiments E1-E20 of Formula (I), wherein A-(R^{A})ₙ is selected from the group consisting of:

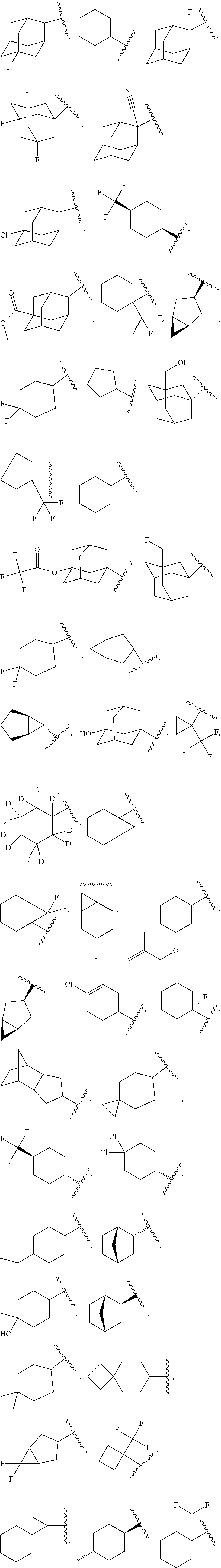

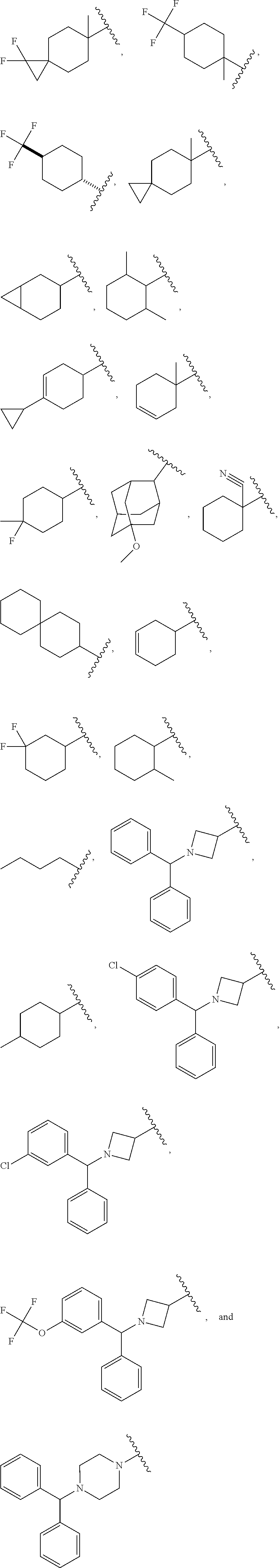

In another embodiment E60, the invention provides a compound or pharmaceutically acceptable salt of embodiments E1-E20 of Formula (I), wherein A-(R^{A})ₙ is selected from the group consisting of:

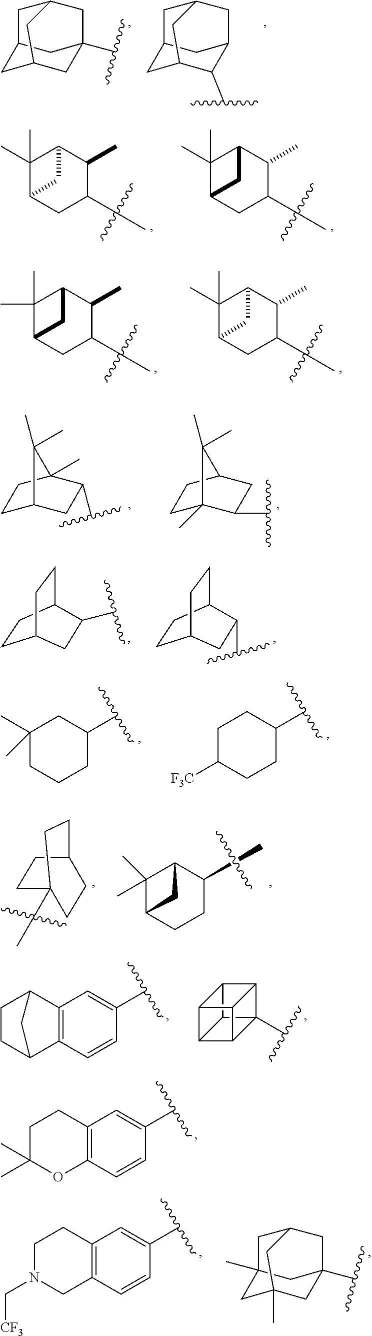

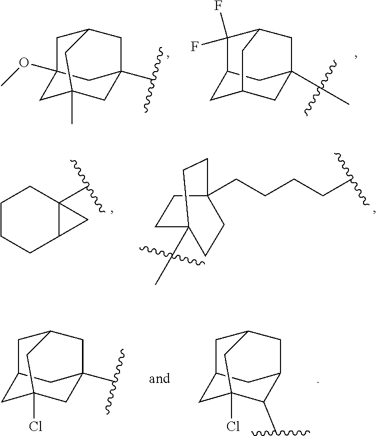 [SMILES: *C12CC3CC(C)(C1)CC(OC)(C3)C2 ; *C12CC3CC(C1)C(F)(F)C(C3)C2 ; *C12CCCCC1C2 ; *CCCCC12CCC(*)(CC1)CC2 ; *C12CC3CC(C1)CC(Cl)(C3)C2 ; *C1C2CC3CC(C2)CC1(Cl)C3]

In another embodiment E61, the invention provides a compound or pharmaceutically acceptable salt of embodiments E1-E20 of Formula (I), wherein the group

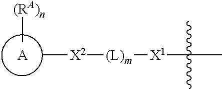

is selected from the group consisting of:

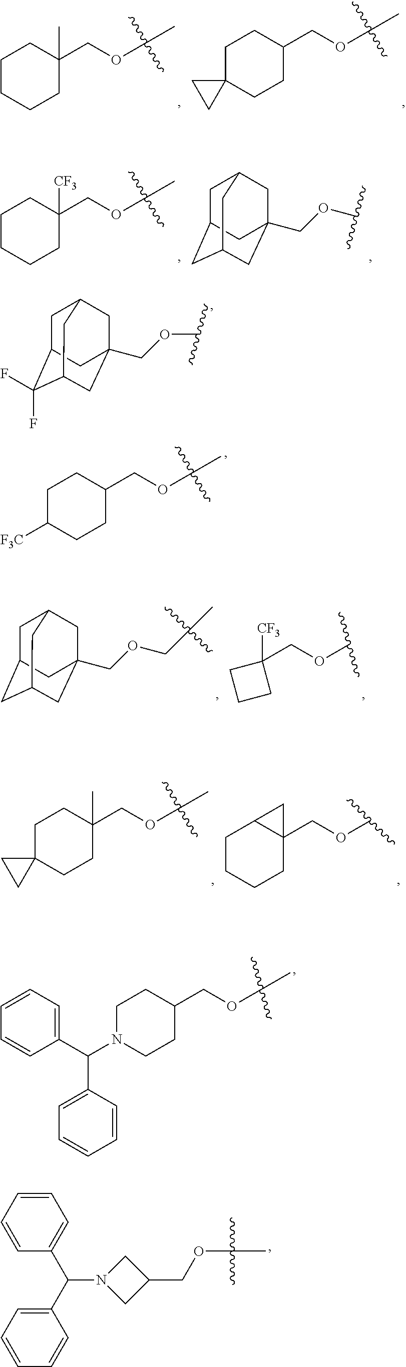

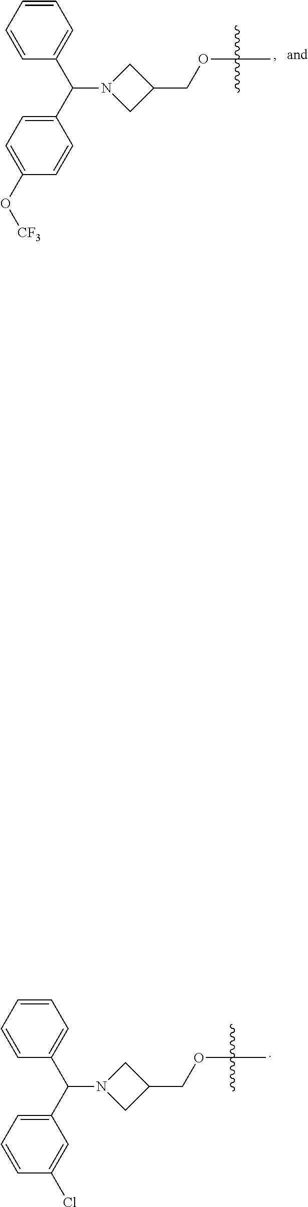 [SMILES: *OCC1CN(C(c2ccccc2)c2ccc(OC(F)(F)F)cc2)C1 ; *OCC1CN(C(c2ccccc2)c2cccc(Cl)c2)C1]

In another embodiment E62, the invention provides a compound or pharmaceutically acceptable salt of embodiments E1-E20 of Formula (I), wherein A(R^{A})ₙ is selected from the group consisting of:

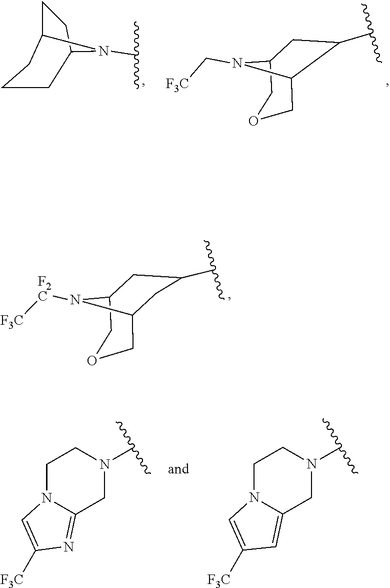 [SMILES: *N1C2CCCC1CC2 ; *C1CC2COCC1N2CC(F)(F)F ; *C1CC2COCC(C1)N2C(F)(F)C(F)(F)F ; *N1CCn2cc(C(F)(F)F)nc2C1 ; *N1CCn2cc(C(F)(F)F)cc2C1]

In another aspect the present invention provides for a pharmaceutical composition comprising a compound of formulae (I), (II), and (III), as described herein or a pharmaceutically acceptable salt thereof, and a pharmaceutically acceptable excipient.

In another aspect the present invention provides for a method of treating a disease or condition in a mammal selected from the group consisting of pain, depression, cardiovascular diseases, respiratory diseases, and psychiatric diseases, and combinations thereof, wherein the method comprises administering to the mammal in need thereof a therapeutically effective amount of a compound as described herein, or a pharmaceutically acceptable salt thereof. In another aspect of the present invention said disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, persistent pain, peripherally mediated pain, centrally mediated pain, chronic headache, migraine headache, sinus headache, tension headache, phantom limb pain, dental pain, peripheral nerve injury or a combination thereof. In another aspect of the present invention said disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritis, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythromelalgia, primary erythromelalgia, familial rectal pain, cancer, epilepsy, partial and general tonic seizures, restless leg syndrome, arrhythmias, fibromyalgia, neuroprotection under ischaemic conditions cause by stroke or neural trauma, tach-arrhythmias, atrial fibrillation and ventricular fibrillation.

In another aspect the present invention provides for a method of treating pain in a mammal by the inhibition of ion flux through a voltage-dependent sodium channel in the mammal, wherein the method comprises administering to the mammal in need thereof a therapeutically effective amount of a compound as described herein, or a pharmaceutically acceptable salt thereof.

In another aspect the present invention provides for a method of decreasing ion flux through a voltage-dependent sodium channel in a cell in a mammal, wherein the method comprises contacting the cell with a compound as described herein, or a pharmaceutically acceptable salt thereof.

In another aspect the present invention provides for a method of treating pruritus in a mammal, wherein the method comprises administering to the mammal in need thereof a therapeutically effective amount of a compound of the invention, or a pharmaceutically acceptable salt thereof.

In another aspect the present invention provides for a method of treating cancer in a mammal, wherein the method comprises administering to the mammal in need thereof a therapeutically effective amount a compound as described herein, or a pharmaceutically acceptable salt thereof.

In another aspect the present invention provides for a method of treating, but not preventing, pain in a mammal, wherein the method comprises administering to the mammal in need thereof a therapeutically effective amount of a compound as described herein, or a pharmaceutically acceptable salt thereof. In another aspect of the present invention the pain is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, persistent pain, peripherally mediated pain, centrally mediated pain, chronic headache, migraine headache, sinus headache, tension headache, phantom limb pain, dental pain, peripheral nerve injury or a combination thereof. In another aspect the present invention the pain is associated with a disease or condition selected from the group consisting of HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritis, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythromelalgia, primary erythromelalgia, familial rectal pain, cancer, epilepsy, partial and general tonic seizures, restless leg syndrome, arrhythmias, fibromyalgia, neuroprotection under ischaemic conditions cause by stroke or neural trauma, tach-arrhythmias, atrial fibrillation and ventricular fibrillation.

In another aspect the present invention provides for a method for the treatment or prophylaxis of pain, depression, cardiovascular disease, respiratory disease, or psychiatric disease, or a combinations thereof, in an animal which method comprises administering an effective amount of a compound of as described herein, or a pharmaceutically acceptable salt thereof.

In another aspect the present invention provides for a compound as described herein, or a pharmaceutically acceptable salt thereof for the use as a medicament for the treatment of diseases and disorders selected from the group consisting of pain, depression, cardiovascular diseases, respiratory diseases, and psychiatric diseases, or a combination thereof.

In another aspect the present invention provides for the use of a compound as described herein, or a pharmaceutically acceptable salt thereof for the manufacture of a medicament for the treatment of diseases and disorders selected from the group consisting of pain, depression, cardiovascular diseases, respiratory diseases, and psychiatric diseases, or a combination thereof.

In another aspect the present invention includes methods of designing NaV1.7 inhibitors utilizing computational methods that utilize virtual “docking” of test molecules into a computer model of the binding site.

A still further aspect of the invention is a method of identifying a compound that binds to the NaV1.7 receptor, wherein the method comprises: modeling test compounds that fit spatially into a NaV1.7 binding site using an atomic structural model of the NaV1.7 receptor binding site or portion thereof, screening the test compounds in an assay, for example a biological assay, characterized by measuring binding of a test compound to the NaV1.7 receptor, and identifying a test compound that binds according to a threshold, such as at least 1 micromolar, at least 0.1 micromolar, at least 0.001 micromolar. The atomic structural model comprises atomic coordinates of a NaV1.7 receptor and may additionally comprise coordinates of a ligand bound to the NaV1.7 binding site. It is to be understood that the atomic structural model may comprise the entire Nav1.7 receptor, or simply a sufficient portion thereof that comprises coordinates of the binding site.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows attempts to rationalize the binding behavior of two classes of NaV1.7 binding compounds from the prior art.

FIGS. 2A and 2B show schematics of binding poses of aryl- and acyl-sulfonamide compounds of the prior art.

FIG. 3A shows a schematic of design of compounds of the present invention, and FIG. 3B shows highlighted portions of the NaV1.7 VSD4 binding pocket and binding poses for 3 classes of compound.

FIG. 4 shows exemplary structural components of compounds of the present invention.

FIGS. 5A, 5B and 5C show a schematic of the binding configuration of the compound of Example 59 from an extracellular view, and an arginine core view, and showing the reduced extent of interaction between ligand and Try 1537.

FIGS. 6A (“Cyclopropane substitution expectedly improves potency”), 6B (“Lipophilic piece can be transposed from one location to another”) and 6C (“compounds are inactive without lipophilic piece”) show schematics of aspects of SAR based on hybrid structured molecules.

FIGS. 7A, 7B, 7C, 7D, and 7E show instances of aryl-sulfonamide binding that exhibit preferential interactions to acyl-sulfonamide compounds.

FIGS. 8A and 8B, illustrate aspects of designing molecules to compensate for the energetic penalty associated with helix rearrangement upon binding, within the VSD4 binding domain.

FIG. 9 panels A and B show cryoEM data from Example 403 for the compound of Example 96: A: Sample freezing; B: 2D classification.

FIG. 10 shows data from cryo-EM analyses in Example 403.

### DETAILED DESCRIPTION

### Definitions

As used herein, the term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like.

The term “alkoxy” is used in its conventional sense, and refers to an alkyl group attached to the remainder of the molecule via an oxygen atom (“oxy”).

The term “alkylthio” is used in its conventional sense, and refers to an alkyl group attached to the remainder of the molecule via a sulfur atom.

The terms “halo” by itself or as part of another substituent, mean, unless otherwise stated, a fluorine, chlorine, bromine, or iodine atom.

The term “haloalkyl” refers to an alkyl that is substituted with one or more (e.g. 1, 2, 3, 4, 5, or 6) halo groups. For example the term includes an alkyl group having 1-6 carbon atoms that is substituted with one or more halo groups. Non-limiting examples of the term C₁-C₆ haloalkyl include fluoromethyl, difluoromethyl, trifluoromethyl, chloromethyl, and 2,2,2-trifluoroethyl.

The term “(haloalkyl)thio” refers to an alkyl that is substituted with one or more (e.g. 1, 2, 3, 4, 5, or 6) halo groups and is attached to the remainder of the molecule via a sulfur atom.

The term “halocycloalkyl” refers to a cycloalkyl that is substituted with one or more (e.g. 1, 2, 3, 4, 5, or 6) halo groups. For example the term includes a cycloalkyl group having 3-6 carbon atoms that is substituted with one or more halo groups. Non-limiting examples of the term C₁-C₆ halocycloalkyl include 1-fluorocyclopropyl.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, naphthyl, 1, 2, 3, 4-tetrahydronaphthyl, anthracenyl, and the like.

The term “(aryl)alkyl” as used herein refers to an aryl group that is attached through an alkyl group (e.g., benzyl or phenethyl).

The term “carbocycle” or “carbocyclyl” refers to a single saturated (i.e., cycloalkyl) or a single partially unsaturated (e.g., cycloalkenyl, cycloalkadienyl, etc.) all carbon ring having 3 to 7 carbon atoms (i.e., (C₃-C₇)carbocycle). The term “carbocycle” or “carbocyclyl” also includes multiple condensed, saturated and partially unsaturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 6 to 20 or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4.5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). The “carbocycle” or “carbocyclyl” can also be optionally substituted with one or more (e.g., 1, 2 or 3) oxo groups. In one embodiment the term carbocycle includes a C₃₋₁₂ carbocycle. In one embodiment the term carbocycle includes a C₃₋₈ carbocycle. In one embodiment the term carbocycle includes a C₃₋₆ carbocycle. In one embodiment the term carbocycle includes a C₃₋₅ carbocycle. Non-limiting examples of carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, bicyclo[2.2.1]heptane, pinane, adamantane, norborene, spirocyclic C₅₋₁₂ alkane, and 1-cyclohex-3-enyl. In one embodiment the term “cycloalkyl” refers to a single saturated all carbon ring having 3 to 8 carbon atoms. Non-limiting examples of carbocycles include cyclopropyl, cyclobutyl, cyclopentyl, and cyclohexyl.

The term “(C₃₋₈cycloalkyl)C₁₋₈ alkyl” refers to a (C₃₋₈cycloalkyl) group that is attached through an alkyl group (e.g., cyclopropylmethyl or 2-cyclopropylethyl).

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example a naphthyridinyl such as 1,8-naphthyridinyl), heterocycles. (to form for example a 1, 2, 3, 4-tetrahydronaphthyridinyl such as 1,2,3,4-tetrahydro-1,8-naphthyridinyl), carbocycles (to form for example 5.6.7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 20 or about 5 to 15 or about 5 to 10 members within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system. It is also to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b, 4,4a, 5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The term “(heteroaryl)C₁₋₈ alkyl” refers to a (heteroaryl) group that is attached through an alkyl group (e.g., pyrid-2-ylmethyl or 2-(pyrid-2-yl)ethyl).

The term “heterocycloalkyl.” “heterocyclic,” or “heterocycle” refers to a saturated or partially unsaturated ring system radical having from 3-10 ring atoms (e.g., 3-10 membered heterocycloalkyl is a heterocycloalkyl radical with 3-10 ring atoms, a C₂₋₉ heterocycloalkyl is a heterocycloalkyl having 3-10 ring atoms with 2-9 ring atoms being carbon) that contain from one to five heteroatoms independently selected from N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, spirocyclic or a polycylic ring system. Non limiting examples of “heterocycloalkyl.” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, tropane, 2-azaspiro[3.3]heptane, (1R,5S)-3-azabicyclo[3.2.1]octane, (1s, 4s)-2-azabicyclo[2.2.2]octane, (1R,4R)-2-oxa-5-azabicyclo[2.2.2]octane and the like A “heterocycloalkyl,” “heterocyclic,” or “heterocycle” group can be attached to the remainder of the molecule through one or more ring carbons or heteroatoms. A “heterocycloalkyl,” “heterocyclic,” or “heterocycle” can include mono- and poly-halogenated variants thereof.

The term “(heterocycloalkyl)C₁₋₈ alkyl” refers to a (heterocycloalkyl) group that is attached through an alkyl group (e.g., pyrrolidin-2-ylmethyl or 2-(pyrrolidine-2-yl)ethyl)

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein a way line “ [symbol] ” that intersects a bond in a chemical structure indicates the point of attachment of the bond that the wavy bond intersects in the chemical structure to the remainder of a molecule.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley and Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxy carbonyl (BOC), benzyloxycarbonyl (CBZ) and 9-fluorenylmethylenoxy carbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4th edition, Wiley-Interscience, New York, 2006.

As used herein, the term “mammal” includes, but is not limited to, humans, mice, rats, guinea pigs, monkeys, dogs, cats, horses, cows, pigs, and sheep.

As used herein, the term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, omithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present invention provides for metabolites of compounds of the invention. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

### Pharmaceutical Compositions and Administration

In addition to one or more of the compounds provided above (or stereoisomers, geometric isomers, tautomers, solvates, metabolites, isotopes, pharmaceutically acceptable salts, or prodrugs thereof), the invention also provides for compositions and medicaments comprising a compound of the invention and at least one pharmaceutically acceptable carrier, diluent or excipient. The compositions of the invention can be used to selectively inhibit NaV1.7 in patients (e.g, humans).

The term “composition,” as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

In one embodiment, the invention provides for pharmaceutical compositions (or medicaments) comprising a compound as described herein, and its stereoisomers, geometric isomers, tautomers, solvates, metabolites, isotopes, pharmaceutically acceptable salts, or prodrugs thereof) and a pharmaceutically acceptable carrier, diluent or excipient. In another embodiment, the invention provides for preparing compositions (or medicaments) comprising compounds of the invention. In another embodiment, the invention provides for administering a compound of the invention or a and compositions comprising a compound of the invention to a patient (e.g., a human patient) in need thereof.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The effective amount of the compound to be administered will be governed by such considerations, and is the minimum amount necessary to inhibit NaV1.7 activity as required to prevent or treat the undesired disease or disorder, such as for example, pain. For example, such amount may be below the amount that is toxic to normal cells, or the mammal as a whole.

In one example, the therapeutically effective amount of the compound of the invention administered parenterally per dose will be in the range of about 0.01-100 mg/kg, alternatively about e.g., 0.1 to 20 mg/kg of patient body weight per day, with the typical initial range of compound used being 0.3 to 15 mg/kg/day. The daily does is, in certain embodiments, given as a single daily dose or in divided doses two to six times a day, or in sustained release form. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, intracerebral, intraocular, intralesional or subcutaneous administration.

The compositions comprising compounds as described herein or an embodiment thereof are normally formulated in accordance with standard pharmaceutical practice as a pharmaceutical composition. A typical formulation is prepared by mixing a compound of the present invention and a diluent, carrier or excipient. Suitable diluents, carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams and Wilkins, 2004; Gennaro, Alfonso R., et al. Remington: The Science and Practice of Pharmacy. Philadelphia: Lippincott, Williams and Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which a compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). A active pharmaceutical ingredient of the invention (e.g., a compound or pharmaceutically acceptable salt of the invention) can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy: Remington the Science and Practice of Pharmacy (2005) 21^{st} Edition, Lippincott Williams and Wilkins, Philadelphia, PA.

Sustained-release preparations of a compound can be prepared. Suitable examples of sustained-release preparations include semipermeable matrices of solid hydrophobic polymers containing a compound as described herein, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate (Sidman et al., Biopolymers 22:547, 1983), non-degradable ethylene-vinyl acetate (Langer et al., J. Biomed. Mater. Res. 15:167, 1981), degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid (EP 133,988A). Sustained release compositions also include liposomally entrapped compounds, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy.

The formulations include those suitable for the administration routes detailed herein. The formulations can conveniently be presented in unit dosage form and can be prepared by any of the methods well known in the art of pharmacy. Techniques and formulations generally are found in Remington: The Science and Practice of Pharmacy: Remington the Science and Practice of Pharmacy (2005) 21^{st} Edition, Lippincott Williams and Wilkins, Philadelphia, PA. Such methods include the step of bringing into association the active ingredient with the carrier which constitutes one or more accessory ingredients.

In general the formulations are prepared by uniformly and intimately bringing into association the active ingredient with liquid carriers, diluents or excipients or finely divided solid carriers, diluents or excipients, or both, and then, if necessary, shaping the product. A typical formulation is prepared by mixing a compound of the present invention and a carrier, diluent or excipient. The formulations can be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. A compound of the present invention is typically formulated into pharmaceutical dosage forms to provide an easily controllable dosage of the drug and to enable patient compliance with the prescribed regimen.

In one example, compounds as described herein may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of the invention is formulated in an acetate buffer, at pH 5. In another embodiment, a compound of the invention is sterile. The compound may be stored, for example, as a solid or amorphous composition, as a lyophilized formulation or as an aqueous solution.

Formulations of a compound as described herein suitable for oral administration can be prepared as discrete units such as pills, capsules, cachets or tablets each containing a predetermined amount of a compound of the invention.

Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs can be prepared for oral use. Formulations of a compound as described herein intended for oral use can be prepared according to any method known to the art for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

An example of a suitable oral administration form is a tablet containing about 1 mg, 5 mg, 10 mg, 25 mg, 30 mg, 50 mg, 80 mg, 100 mg, 150 mg, 250 mg, 300 mg and 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredient can be employed with either a paraffinic or a water-miscible ointment base. Alternatively, the active ingredients can be formulated in a cream with an oil-in-water cream base. If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of the active ingredient through the skin or other affected areas. Examples of such dermal penetration enhancers include dimethyl sulfoxide and related analogs.

The oily phase of the emulsions of this invention can be constituted from known ingredients in a known manner. While the phase can comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

In one aspect of topical applications, it is desired to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

Aqueous suspensions of a compound as described herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose or saccharin.

Formulations of a compound as described herein can be in the form of a sterile injectable preparation, such as a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The amount of active ingredient that can be combined with the carrier material to produce a single dosage form will vary depending upon the host treated and the particular mode of administration. For example, a time-release formulation intended for oral administration to humans can contain approximately 1 to 1000 mg of active material compounded with an appropriate and convenient amount of carrier material which can vary from about 5 to about 95% of the total compositions (weight:weight). The pharmaceutical composition can be prepared to provide easily measurable amounts for administration. For example, an aqueous solution intended for intravenous infusion can contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration can be prepared according to conventional methods and can be delivered with other therapeutic agents such as compounds heretofore used in the treatment of disorders as described below.

The formulations can be packaged in unit-dose or multi-dose containers, for example sealed ampoules and vials, and can be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

When the binding target is located in the brain, certain embodiments of the invention provide for a compound as described herein to traverse the blood-brain barrier. Certain neurodegenerative diseases are associated with an increase in permeability of the blood-brain barrier, such that a compound of the invention can be readily introduced to the brain. When the blood-brain barrier remains intact, several art-known approaches exist for transporting molecules across it, including, but not limited to, physical methods, lipid-based methods, and receptor and channel-based methods.

Physical methods of transporting a compound as described herein across the blood-brain barrier include, but are not limited to, circumventing the blood-brain barrier entirely, or by creating openings in the blood-brain barrier.

Circumvention methods include, but are not limited to, direct injection into the brain (see, e.g., Papanastassiou et al., Gene Therapy 9:398-406, 2002), interstitial infusion/convection-enhanced delivery (see, e.g., Bobo et al., Proc. Natl. Acad. Sci. U.S.A. 91:2076-2080, 1994), and implanting a delivery device in the brain (see, e.g., Gill et al., Nature Med. 9:589-595, 2003; and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Volumes 1 and 2, Plenum Press, N.Y., 1989)), and permeabilization by, e.g., bradykinin or permeabilizer A-7 (see, e.g., U.S. Pat. Nos. 5,112,596, 5,268,164, 5,506,206, and 5,686,416).

Lipid-based methods of transporting a compound as described herein across the blood-brain barrier include, but are not limited to, encapsulating the a compound as described herein in liposomes that are coupled to antibody binding fragments that bind to receptors on the vascular endothelium of the blood-brain barrier (see, e.g., U.S. Patent Application Publication No. 2002/0025313), and coating a compound as described herein in low-density lipoprotein particles (see, e.g., U.S. Patent Application Publication No. 2004/0204354) or apolipoprotein E (see, e.g., U.S. Patent Application Publication No. 2004/0131692).

Receptor and channel-based methods of transporting a compound as described herein across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating a compound as described herein with a transferrin and modulating activity of the one or more transferrin receptors (see, e.g., U.S. Patent Application Publication No. 2003/0129186), and cationizing the antibodies (see, e.g., U.S. Pat. No. 5,004,697).

For intracerebral use, in certain embodiments, the compounds can be administered continuously by infusion into the fluid reservoirs of the CNS, although bolus injection may be acceptable. The inhibitors can be administered into the ventricles of the brain or otherwise introduced into the CNS or spinal fluid. Administration can be performed by use of an indwelling catheter and a continuous administration means such as a pump, or it can be administered by implantation, e.g., intracerebral implantation of a sustained-release vehicle. More specifically, the inhibitors can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer's disease patients and animal models for Parkinson's disease, as described by Harbaugh, J. Neural Transm. Suppl. 24:271, 1987; and DeYebenes et al, Mov. Disord. 2: 143, 1987.

A compound as described herein used in the invention are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. A compound as described herein need not be, but is optionally formulated with one or more agent currently used to prevent or treat the disorder in question. The effective amount of such other agents depends on the amount of a compound of the invention present in the formulation, the type of disorder or treatment, and other factors discussed above.

These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a compound as described herein (when used alone or in combination with other agents) will depend on the type of disease to be treated, the properties of the compound, the severity and course of the disease, whether the compound is administered for preventive or therapeutic purposes, previous therapy, the patient's clinical history and response to the compound, and the discretion of the attending physician. The compound is suitably administered to the patient at one time or over a series of treatments. Depending on the type and severity of the disease, about 1 μg/kg to 15 mg/kg (e.g., 0.1 mg/kg-10 mg/kg) of compound can be an initial candidate dosage for administration to the patient, whether, for example, by one or more separate administrations, or by continuous infusion. One typical daily dosage might range from about 1 μg kg to 100 mg/kg or more, depending on the factors mentioned above. For repeated administrations over several days or longer, depending on the condition, the treatment would generally be sustained until a desired suppression of disease symptoms occurs. One exemplary dosage of a compound of the invention would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. An exemplary dosing regimen comprises administering an initial loading dose of about 4 mg/kg, followed by a weekly maintenance dose of about 2 mg kg of the compound. However, other dosage regimens may be useful. The progress of this therapy is easily monitored by conventional techniques and assays.

Other typical daily dosages might range from, for example, about 1 g/kg to up to 100 mg/kg or more (e.g., about 1 μg kg to 1 mg/kg, about 1 μg/kg to about 5 mg/kg, about 1 mg kg to 10 mg/kg, about 5 mg/kg to about 200 mg/kg, about 50 mg/kg to about 150 mg/mg, about 100 mg/kg to about 500 mg/kg, about 100 mg/kg to about 400 mg/kg, and about 200 mg/kg to about 400 mg/kg), depending on the factors mentioned above. Typically, the clinician will administer a compound until a dosage is reached that results in improvement in or, optimally, elimination of, one or more symptoms of the treated disease or condition. The progress of this therapy is easily monitored by conventional assays. One or more agent provided herein may be administered together or at different times (e.g., one agent is administered prior to the administration of a second agent). One or more agent may be administered to a subject using different techniques (e.g., one agent may be administered orally, while a second agent is administered via intramuscular injection or intranasally). One or more agent may be administered such that the one or more agent has a pharmacologic effect in a subject at the same time. Alternatively, one or more agent may be administered, such that the pharmacological activity of the first administered agent is expired prior the administration of one or more secondarily administered agents (e.g., 1, 2, 3, or 4 secondarily administered agents).

### Synthesis of Compounds

Compounds of formula I, II, and III can be prepared using starting materials, synthetic processes and synthetic intermediates like those described in the Examples below. In particular, compounds of formula I can be prepared as illustrated in Schemes 1 and 2; compounds of formula II can be prepared as illustrated in Scheme 3; and compounds of formula III can be prepared as illustrated in Schemes 4 and 5.

Compounds of formula (I), wherein X¹ is O, S, or NH, may be prepared by the process illustrated in Scheme 1.

Compounds of formula (I) can be made from compounds of formula (11) by displacement with formula (III) and a base (reaction step ii in Scheme 1). Suitable conditions include potassium tert-butoxide in DMSO, NaH in DMF or K₂CO₃ in DMF. Formula (II) can be made according to step (i) by activation of the acid group of formula (IV) with reagents such as oxalyl chloride, carbonyl di-imidazole (CDI), propylphosphonic anhydride, a uronium based amide coupling agent or a carbodiimide reagent followed by displacement with a sulfonamide of formula (VII) in the presence of a nucleophilic base such as 4-dimethylaminopyridine. Illustrative conditions comprise N, N-dimethylaminopropyl-N-ethylcarbodiimide and 4-dimethylaminopyridine with N, N-diisopropylethylamine.

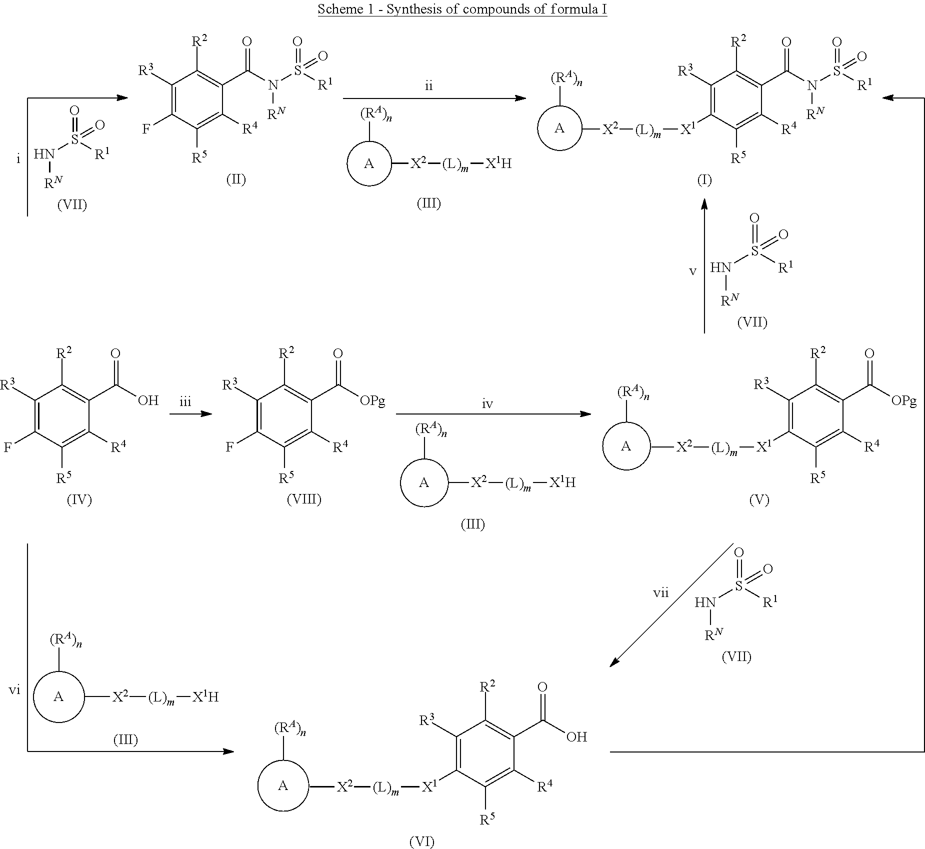

Alternatively, compounds of formula (I) can be made from compounds of formula (IV) by reversing steps (i) and (ii) as described in Scheme 1. Illustrative conditions for steps vi and vii are as previously described in steps (ii) and (i), respectively.

Compounds of formula (I) can also be made from compounds of formula (V) according to step (v) by displacement of the ester with compounds of formula (VII) and a suitable base such as potassium tert-butoxide, NaH or DBU. Compounds of formula (I) can also be made from compounds of formula (v) by a two steps sequence (see steps viii and vii in Scheme 1). Compounds of formula (V) can be made from compounds of formula (VIII) according to step (iv) via a nucleophilic substitution reaction using compounds of formula (III) and a base as described in step ii. Compounds of formula (VIII) can be made from compounds of formula (IV) according to step (iii) using protecting group methodology as described in references such as ‘Greene's Protective Groups in Organic Synthesis’. When Pg is tolyl, illustrative conditions comprise thionyl chloride or carbonyldiimidazole with para-cresol. When Pg is tert-butyl, illustrative conditions comprise di-tert butyl dicarbonate and 4-dimethylaminopyridine in tert-butanol.

Compounds of formula (I), wherein R⁵ is Ar, heteroaryl, C₁₋₈ alkyl, C₁₋₈ haloalkyl, C₁₋₈ alkoxy, C₃₋₁₀ cycloalkyl or C₂₋₉ heterocycloalkyl can be prepared by the process illustrated in Scheme 2. In certain embodiment, W groups in compounds of formula (IX, X and XI) are an ester or cyano group.

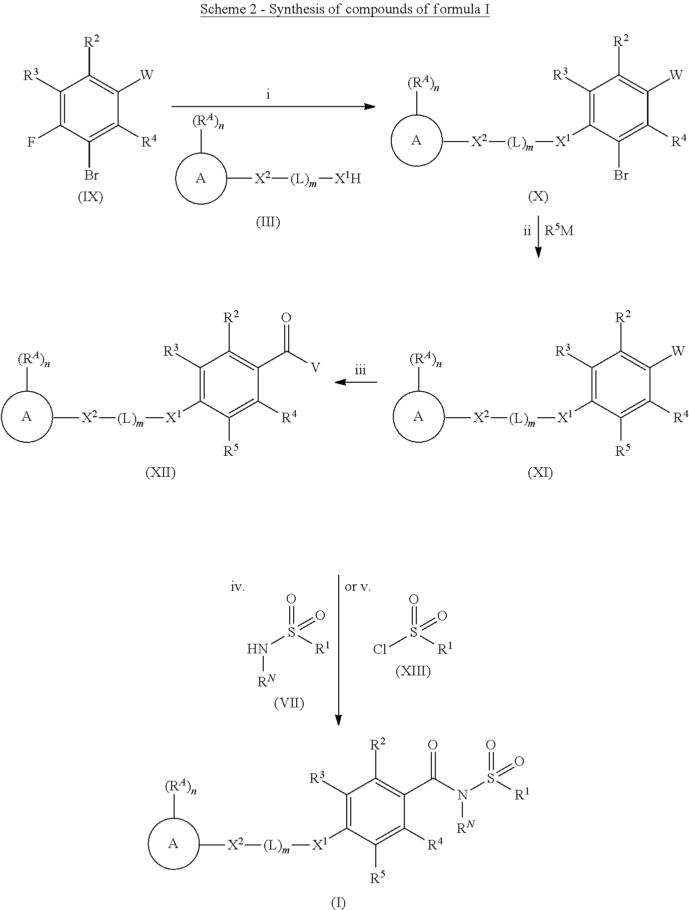

Compounds of formula (I) can be prepared from compounds of formulae (XII) (—V═OH) according to reaction step (iv) by activation of the acid group with reagents such as oxalyl chloride, carbonyl di-imidazole (CDI), a uronium based amide coupling agent, propylphosphonic anhydride or a carbodiimide reagent followed by displacement with a suitable sulfonamide of formula (VII) in the presence of a nucleophilic base such as 4-dimethylaminopyridine.

Alternatively, compounds of formula (I) can be prepared from compounds of formula (XII) (—V═NH₂) according to reaction step (v) by displacement of a sulfonyl chloride of formula (XIII) under basic reaction conditions.

Compounds of formula (XII) can be prepared by hydrolysis of the nitrile functional group in compounds of formula (XI, W═CN) or by hydrolysis of the ester functional group in compounds of formula (XI, W═CO₂Pg) by either acidic or basic methods according to step (iii) as required.

Compounds of formula (XI) can be prepared from compounds of formula (X) by palladium-catalyzed coupling of a compound of formula (R₅M) according to step (ii).

Conveniently the coupling is effective with a boronic acid or ester of formula (R₅M). The coupling reaction can be carried out with a variety of palladium catalysts such as palladium acetate or tetrakistriphenylphosphine palladium (0) in various solvents and in the presence of bases such as sodium and potassium carbonate, cesium fluoride or potassium phosphate. Compounds of formula (X) can be prepared under similar conditions as described for the preparation of compounds of formula (V), (VI) and (I) in Scheme 1.

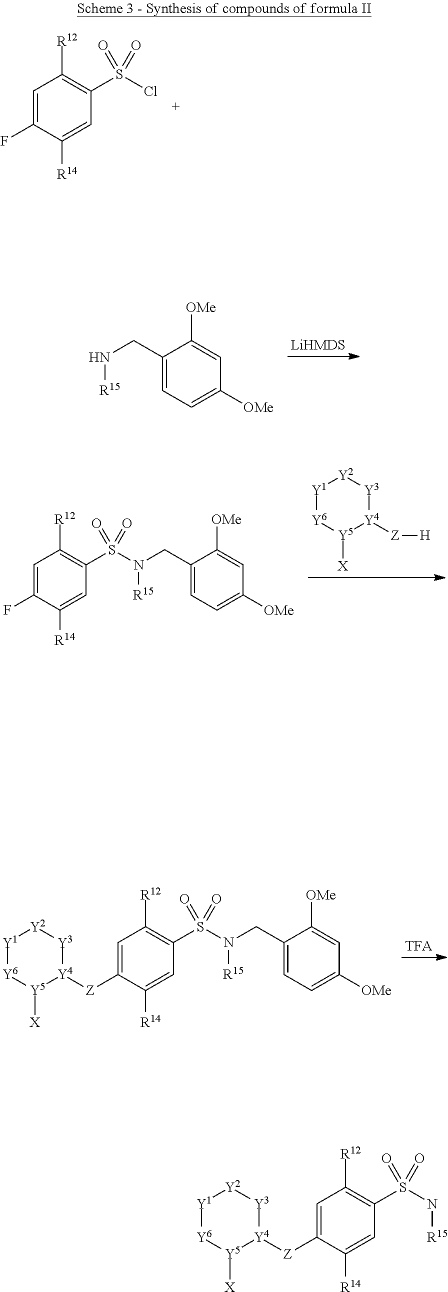

Compounds of formula III may be prepared by the processes illustrated in Scheme 4.

A compound of Formula III can be prepared by treating an amine of Formula IIIa with a sulfonylating reagent, e.g., a reagent of formula X—SO₂—R³¹ wherein X is a suitable leaving group, such as chloro, to provide the compound of Formula I. Accordingly, the invention also provides novel amines of Formula IIIa, which are useful intermediates for preparing the corresponding sulfonamides of Formula III. The invention also provides a method for preparing a compound of Formula III comprising treating a corresponding amine of Formula IIIa with corresponding sulfonylating reagent to provide the compound of Formula III.

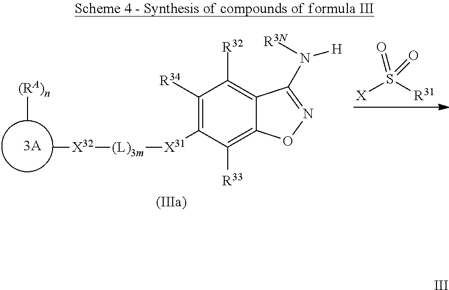

An intermediate amine of Formula IIIa wherein R^{N} is H can be prepared by treating a cyano fluoride of Formula IIIb with N-hydroxyacetamide as illustrated in Scheme 5.

Amines of Formula IIIa wherein R^{N} is H are general intermediates that can be converted to compounds of formula III using standard techniques. Accordingly, the invention also provides novel amines of Formula IIIa wherein R^{N} is H as well as novel compounds of Formula IIIb, which are useful intermediates for preparing the corresponding sulfonamides of Formula III. The invention also provides a method for preparing a compound of Formula IIIa, wherein R^{N} is H comprising treating a corresponding amine of Formula IIIb with N-hydroxyacetamide to provide the compound of Formula IIIa.

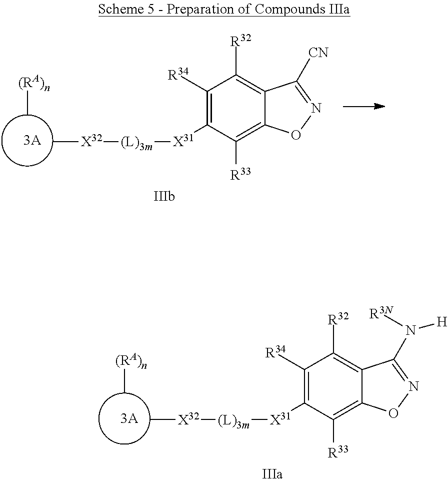

### Indications and Methods of Treatment

The compounds of the invention modulate, preferably inhibit, ion flux through a voltage-dependent sodium channel in a mammal, (e.g, a human). Any such modulation, whether it be partial or complete inhibition or prevention of ion flux, is sometimes referred to herein as “blocking” and corresponding compounds as “blockers” or “inhibitors”. In general, the compounds of the invention modulate the activity of a sodium channel downwards by inhibiting the voltage-dependent activity of the sodium channel, and/or reduce or prevent sodium ion flux across a cell membrane by preventing sodium channel activity such as ion flux.

Accordingly, the compounds of the invention are sodium channel blockers and are therefore useful for treating diseases and conditions in mammals, for example humans, and other organisms, including all those diseases and conditions which are the result of aberrant voltage-dependent sodium channel biological activity or which may be ameliorated by modulation of voltage-dependent sodium channel biological activity. In particular, the compounds of the invention, i.e., the compounds of formula (I) and embodiments and (or stereoisomers, geometric isomers, tautomers, solvates, metabolites, isotopes, pharmaceutically acceptable salts, or prodrugs thereof), are useful for treating diseases and conditions in mammals, for example humans, which are the result of aberrant voltage-dependent NaV1.7 biological activity or which may be ameliorated by the modulation, preferably the inhibition, of NaV1.7 biological activity. In certain aspects, the compounds of the invention selectively inhibit NaV1.7 over NaV1.5.

As defined herein, a sodium channel-mediated disease or condition refers to a disease or condition in a mammal, preferably a human, which is ameliorated upon modulation of the sodium channel and includes, but is not limited to, pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome.

In one aspect, the present invention relates to compounds, pharmaceutical compositions and methods of using the compounds and pharmaceutical compositions for the treatment of sodium channel-mediated diseases in mammals, preferably humans and preferably diseases and conditions related to pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome, by administering to a mammal, for example a human, in need of such treatment an effective amount of a sodium channel blocker modulating, especially inhibiting, agent.

A sodium channel-mediated disease or condition also includes pain associated with HIV, HIV treatment induced neuropathy, trigeminal neuralgia, glossopharyngeal neuralgia, neuropathy secondary to metastatic infiltration, adiposis dolorosa, thalamic lesions, hypertension, autoimmune disease, asthma, drug addiction (e.g., opiate, benzodiazepine, amphetamine, cocaine, alcohol, butane inhalation), Alzheimer, dementia, age-related memory impairment, Korsakoff syndrome, restenosis, urinary dysfunction, incontinence, Parkinson's disease, cerebrovascular ischemia, neurosis, gastrointestinal disease, sickle cell anemia, transplant rejection, heart failure, myocardial infarction, reperfusion injury, intermittant claudication, angina, convulsion, respiratory disorders, cerebral or myocardial ischemias, long-QT syndrome, Catecholeminergic polymorphic ventricular tachycardia, ophthalmic diseases, spasticity, spastic paraplegia, myopathies, myasthenia gravis, paramyotonia congentia, hyperkalemic periodic paralysis, hypokalemic periodic paralysis, alopecia, anxiety disorders, psychotic disorders, mania, paranoia, seasonal affective disorder, panic disorder, obsessive compulsive disorder (OCD), phobias, autism, Aspergers Syndrome, Retts syndrome, disintegrative disorder, attention deficit disorder, aggressivity, impulse control disorders, thrombosis, pre clampsia, congestive cardiac failure, cardiac arrest, Freidrich's ataxia, Spinocerebellear ataxia, myelopathy, radiculopathy, systemic lupus erythamatosis, granulomatous disease, olivo-ponto-cerebellar atrophy, spinocerebellar ataxia, episodic ataxia, myokymia, progressive pallidal atrophy, progressive supranuclear palsy and spasticity, traumatic brain injury, cerebral oedema, hydrocephalus injury, spinal cord injury, anorexia nervosa, bulimia, Prader-Willi syndrome, obesity, optic neuritis, cataract, retinal haemorrhage, ischaemic retinopathy, retinitis pigmentosa, acute and chronic glaucoma, macular degeneration, retinal artery occlusion, Chorea, Huntington's chorea, cerebral edema, proctitis, post-herpetic neuralgia, eudynia, heat sensitivity, sarcoidosis, irritable bowel syndrome, Tourette syndrome, Lesch-Nyhan Syndrome, Brugado syndrome, Liddle syndrome, Crohns disease, multiple sclerosis and the pain associated with multiple sclerosis (MS), amyotrophic lateral sclerosis (ALS), disseminated sclerosis, diabetic neuropathy, peripheral neuropathy, charcot marie tooth syndrome, arthritic, rheumatoid arthritis, osteoarthritis, chondrocalcinosis, atherosclerosis, paroxysmal dystonia, myasthenia syndromes, myotonia, myotonic dystrophy, muscular dystrophy, malignant hyperthermia, cystic fibrosis, pseudoaldosteronism, rhabdomyolysis, mental handicap, hypothyroidism, bipolar depression, anxiety, schizophrenia, sodium channel toxin related illnesses, familial erythromelalgia, primary erythromelalgia, rectal pain, cancer, epilepsy, partial and general tonic seizures, febrile seizures, absence seizures (petit mal), myoclonic seizures, atonic seizures, clonic seizures, Lennox Gastaut, West Syndrome (infantile spasms), multiresistant seizures, seizure prophylaxis (anti-epileptogenic), familial Mediterranean fever syndrome, gout, restless leg syndrome, arrhythmias, fibromyalgia, neuroprotection under ischaemic conditions caused by stroke or neural trauma, tachy-arrhythmias, atrial fibrillation and ventricular fibrillation and as a general or local anaesthetic.

As used herein, the term “pain” refers to all categories of pain and is recognized to include, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, chronic regional pain syndrome (CRPS), reflex sympathetic dystrophy, brachial plexus avulsion, neurogenic bladder, acute pain (e.g., musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, and combinations thereof.

Furthermore, sodium channel blockers have clinical uses in addition to pain. The present invention therefore also relates to compounds, pharmaceutical compositions and methods of using the compounds and pharmaceutical compositions for the treatment of diseases or conditions such as cancer and pruritus (itch).

Pruritus, commonly known as itch, is a common dermatological condition. While the exact causes of pruritus are complex and incompletely understood, there has long been evidence that itch involves sensory neurons, especially C fibers, similar to those that mediate pain (Schmelz, M., et al., J. Neurosci. (1997), 17: 8003-8). In particular, it is believed that sodium influx through voltage-gated sodium channels is essential for the propagation of itch sensation from the skin. Transmission of the itch impulses results in the unpleasant sensation that elicits the desire or reflex to scratch.

Multiple causes and electrical pathways for eliciting itch are known. In humans, pruritus can be elicited by histamine or PAR-2 agonists such as mucunain that activate distinct populations of C fibers (Namer, B., et al., J. Neurophysiol. (2008), 100: 2062-9). A variety of neurotrophic peptides are known to mediate itch in animal models (Wang, H., and Yosipovitch, G., International Journal of Dermatology (2010), 49: 1-11). Itch can also be elicited by opioids, evidence of distinct pharmacology from that of pain responses.

There exists a complex interaction between itch and pain responses that arises in part from the overlapping sensory input from the skin (Ikoma, A., et al., Arch. Dermatol. (2003), 139: 1475-8) and also from the diverse etiology of both pain and pruritus. Pain responses can exacerbate itching by enhancing central sensitization or lead to inhibition of painful scratching. Particularly severe forms of chronic itch occur when pain responses are absent, as in the case of post-herpetic itch (Oaklander, A. L., et al., Pain (2002), 96: 9-12).

The compounds of the invention can also be useful for treating pruritus. The rationale for treating itch with inhibitors of voltage-gated sodium channels, especially NaV1.7, is as follows.

The propagation of electrical activity in the C fibers that sense pruritinergic stimulants requires sodium entry through voltage-gated sodium channels.

NaV1.7 is expressed in the C fibers and kerotinocytes in human skin (Zhao, P., et al., Pain (2008), 139: 90-105).

A gain of function mutation of NaV1.7 (L858F) that causes erythromelalgia also causes chronic itch (Li, Y., et al., Clinical and Experimental Dermatology (2009), 34: e313-e4).

Chronic itch can be alleviated with treatment by sodium channel blockers, such as the local anesthetic lidocaine (Oaklander, A. L., et al., Pain (2002), 96: 9-12; Villamil, A. G., et al., The American Journal of Medicine (2005), 118: 1160-3). In these reports, lidocaine was effective when administered either intravenously or topically (a Lidoderm patch). Lidocaine can have multiple activities at the plasma concentrations achieved when administered systemically, but when administered topically, the plasma concentrations are only about 1 μM (Center for Drug Evaluation and Research NDA 20-612). At these concentrations, lidocaine is selective for sodium channel block and inhibits spontaneous electrical activity in C fibers and pain responses in animal models (Xiao, W. H., and Bennett, G. J. Pain (2008), 137: 218-28). The types of itch or skin irritation, include, but are not limited to:
psoriatic pruritus, itch due to hemodyalisis, aguagenic pruritus, and itching caused by skin disorders (e.g., contact dermatitis), systemic disorders, neuropathy, psychogenic factors or a mixture thereof,
itch caused by allergic reactions, insect bites, hypersensitivity (e.g., dry skin, acne, eczema, psoriasis), inflammatory conditions or injury;
itch associated with vulvar vestibulitis; and
skin irritation or inflammatory effect from administration of another therapeutic such as, for example, antibiotics, antivirals and antihistamines.

The compounds of the invention are also useful in treating certain cancers, such as hormone sensitive cancers, such as prostate cancer (adenocarcinoma), breast cancer, ovarian cancer, testicular cancer and thyroid neoplasia, in a mammal, preferably a human. The voltage gated sodium channels have been demonstrated to be expressed in prostate and breast cancer cells. Up-regulation of neonatal NaV1.5 occurs as an integral part of the metastatic process in human breast cancer and could serve both as a novel marker of the metastatic phenotype and a therapeutic target (Clin. Cancer Res. (2005), Aug. 1; 11(15): 5381-9). Functional expression of voltage-gated sodium channel alpha-subunits, specifically NaV1.7, is associated with strong metastatic potential in prostate cancer (CaP) in vitro. Voltage-gated sodium channel alpha-subunits immunostaining, using antibodies specific to the sodium channel alpha subunit was evident in prostatic tissues and markedly stronger in CaP vs non-CaP patients (Prostate Cancer Prostatic Dis., 2005; 8(3):266-73). See also Diss, J. K. J., et al., Mol. Cell. Neurosci. (2008), 37:537-547 and Kis-Toth, K., et al., The Journal of Immunology (2011), 187:1273-1280.

In consideration of the above, in one embodiment, the present invention provides a method for treating a mammal for, or protecting a mammal from developing, a sodium channel-mediated disease, especially pain, comprising administering to the mammal, especially a human, in need thereof, a therapeutically effective amount of a compound of the invention or a pharmaceutical composition comprising a therapeutically effective amount of a compound of the invention wherein the compound modulates the activity of one or more voltage-dependent sodium channels.

In another embodiment of the invention is a method of treating a disease or a condition in a mammal, preferably a human, wherein the disease or condition is selected from the group consisting of pain, depression, cardiovascular diseases, respiratory diseases, and psychiatric diseases, and combinations thereof, and wherein the method comprises administering to the mammal in need thereof a therapeutically effective amount of an embodiment of a compound of the invention, as set forth above, as a stereoisomer, enantiomer or tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer or tautomer thereof or mixtures thereof, or a pharmaceutically acceptable salt, solvate or prodrug thereof, and a pharmaceutically acceptable excipient.

One embodiment of this embodiment is wherein the disease or condition is selected from the group consisting of acute pain, chronic pain, neuropathic pain, inflammatory pain, visceral pain, cancer pain, chemotherapy pain, trauma pain, surgical pain, post surgical pain, childbirth pain, labor pain, neurogenic bladder, ulcerative colitis, persistent pain, peripherally mediated pain, centrally mediated pain, chronic headache, migraine headache, sinus headache, tension headache, phantom limb pain, peripheral nerve injury, and combinations thereof.

Another embodiment of this embodiment is wherein the disease or condition is selected from the group consisting of pain associated with HIV, HIV treatment induced neuropathy, trigeminal neuralgia, post herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythromelalgia, primary erythromelalgia, familial rectal pain, cancer, epilepsy, partial and general tonic seizures, restless leg syndrome, arrhythmias, fibromyalgia, neuroprotection under ischaemic conditions caused by stroke or neural trauma, tachy arrhythmias, atrial fibrillation and ventricular fibrillation.

Another embodiment of the invention is a method of treating, but not preventing, pain in a mammal, wherein the method comprises administering to the mammal in need thereof a therapeutically effective amount of a compound of the invention, as set forth above, as a stereoisomer, enantiomer or tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer or tautomer thereof or mixtures thereof, or a pharmaceutically acceptable salt, solvate or prodrug thereof, and a pharmaceutically acceptable excipient.

One embodiment of this embodiment is a method wherein the pain is selected from the group consisting of neuropathic pain, inflammatory pain, visceral pain, cancer pain, chemotherapy pain, trauma pain, surgical pain, post surgical pain, childbirth pain, labor pain, dental pain, chronic pain, persistent pain, peripherally mediated pain, centrally mediated pain, chronic headache, migraine headache, sinus headache, tension headache, phantom limb pain, peripheral nerve injury, trigeminal neuralgia, post herpetic neuralgia, eudynia, familial erythromelalgia, primary erythromelalgia, familial rectal pain or fibromyalgia, and combinations thereof.

Another embodiment of this embodiment is a method wherein the pain is associated with a disease or condition selected from HIV, HIV treatment induced neuropathy, heat sensitivity, tosarcoidosis, irritable bowel syndrome, Crohns disease, multiple sclerosis, amyotrophic lateral sclerosis, diabetic neuropathy, peripheral neuropathy, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, neurogenic bladder, ulcerative colitis, cancer, epilepsy, partial and general tonic seizures, restless leg syndrome, arrhythmias, ischaemic conditions caused by stroke or neural trauma, tachy arrhythmias, atrial fibrillation and ventricular fibrillation.

Another embodiment of the invention is the method of treating pain in a mammal, preferably a human, by the inhibition of ion flux through a voltage dependent sodium channel in the mammal, wherein the method comprises administering to the mammal in need thereof a therapeutically effective amount of an embodiment of a compound of the invention, as set forth above, as a stereoisomer, enantiomer or tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer or tautomer thereof or mixtures thereof, or a pharmaceutically acceptable salt, solvate or prodrug thereof, and a pharmaceutically acceptable excipient.

Another embodiment of the invention is the method of treating pruritus in a mammal, preferably a human, wherein the method comprises administering to the mammal in need thereof a therapeutically effective amount of an embodiment of a compound of the invention, as set forth above, as a stereoisomer, enantiomer or tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer or tautomer thereof or mixtures thereof, or a pharmaceutically acceptable salt, solvate or prodrug thereof, and a pharmaceutically acceptable excipient.

Another embodiment of the invention is the method of treating cancer in a mammal, preferably a human, wherein the method comprises administering to the mammal in need thereof a therapeutically effective amount of an embodiment of a compound of the invention, as set forth above, as a stereoisomer, enantiomer or tautomer thereof or mixtures thereof, or a pharmaceutically acceptable salt, solvate or prodrug thereof, or a pharmaceutical composition comprising a therapeutically effective amount of a compound of the invention, as set forth above, as a stereoisomer, enantiomer or tautomer thereof or mixtures thereof, or a pharmaceutically acceptable salt, solvate or prodrug thereof, and a pharmaceutically acceptable excipient.

Another embodiment of the invention is the method of decreasing ion flux through a voltage dependent sodium channel in a cell in a mammal, wherein the method comprises contacting the cell with an embodiment of a compound of the invention, as set forth above, as a stereoisomer, enantiomer or tautomer thereof or mixtures thereof, or a pharmaceutically acceptable salt, solvate or prodrug thereof.

Another embodiment of the invention is the method of selectively inhibiting a first voltage-gated sodium channel over a second voltage-gated sodium channel in a mammal, wherein the method comprises administering to the mammal an inhibitory amount of a compound of formula (I), or an embodiment of a compound of formula (I).

Another embodiment of the invention is the method of selectively inhibiting NaV1.7 in a mammal or a mammalian cell as compared to NaV1.5, wherein the method comprises administering to the mammal in need thereof an inhibitory amount of a compound of formula (I) or an embodiment of an embodiment thereof.

For each of the above embodiments described related to treating diseases and conditions in a mammal, the present invention also contemplates relatedly a compound as described herein for the use as a medicament in the treatment of such diseases and conditions.

For each of the above embodiments described related to treating diseases and conditions in a mammal, the present invention also contemplates relatedly the use of a compound as described herein for the manufacture of a medicament for the treatment of such diseases and conditions.

Another embodiment of the invention is a method of using a compound as described herein as a standard or control in in vitro or in vivo assays in determining the efficacy of test compounds in modulating voltage-dependent sodium channels.

In another embodiment of the invention, the compounds as described herein are isotopically-labeled by having one or more atoms therein replaced by an atom having a different atomic mass or mass number. Such isotopically-labeled (i.e., radiolabelled) compounds are considered to be within the scope of this invention. Examples of isotopes that can be incorporated into the compounds include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These isotopically-labeled compounds would be useful to help determine or measure the effectiveness of the compounds, by characterizing, for example, the site or mode of action on the sodium channels, or binding affinity to pharmacologically important site of action on the sodium channels, particularly NaV1.7. Certain isotopically-labeled compounds, for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described in the Examples as set out below using an appropriate isotopically-labeled reagent in place of the non-labeled reagent previously employed.

### Testing Compounds

The assessment of the compounds of the invention in mediating, especially inhibiting, the sodium channel ion flux can be determined using the assays described hereinbelow. Alternatively, the assessment of the compounds in treating conditions and diseases in humans may be established in industry standard animal models for demonstrating the efficacy of compounds in treating pain. Animal models of human neuropathic pain conditions have been developed that result in reproducible sensory deficits (allodynia, hyperalgesia, and spontaneous pain) over a sustained period of time that can be evaluated by sensory testing. By establishing the degree of mechanical, chemical, and temperature induced allodynia and hyperalgesia present, several physiopathological conditions observed in humans can be modeled allowing the evaluation of pharmacotherapies.

In rat models of peripheral nerve injury, ectopic activity in the injured nerve corresponds to the behavioural signs of pain. In these models, intravenous application of the sodium channel blocker and local anesthetic lidocaine can suppress the ectopic activity and reverse the tactile allodynia at concentrations that do not affect general behaviour and motor function (Mao, J. and Chen, L. L, Pain (2000), 87:7-17). Allometric scaling of the doses effective in these rat models, translates into doses similar to those shown to be efficacious in humans (Tanelian, D. L. and Brose, W. G., Anesthesiology (1991), 74(5):949-951). Furthermore, Lidoderm®, lidocaine applied in the form of a dermal patch, is currently an FDA approved treatment for post-herpetic neuralgia (Devers, A. and Glaler, B. S., Clin. J. Pain (2000), 16(3):205-8).

The present invention readily affords many different means for identification of sodium channel modulating agents that are useful as therapeutic agents. Identification of modulators of sodium channel can be assessed using a variety of in vitro and in vivo assays, e.g., measuring current, measuring membrane potential, measuring ion flux, (e.g., sodium or guanidinium), measuring sodium concentration, measuring second messengers and transcription levels, and using e.g., voltage-sensitive dyes, radioactive tracers, and patch-clamp electrophysiology.

One such protocol involves the screening of chemical agents for ability to modulate the activity of a sodium channel thereby identifying it as a modulating agent.

A typical assay described in Bean et al., J. General Physiology (1983), 83:613-642, and Leuwer, M., et al., Br. J. Pharmacol. (2004), 141(1):47-54, uses patch-clamp techniques to study the behaviour of channels. Such techniques are known to those skilled in the art, and may be developed, using current technologies, into low or medium throughput assays for evaluating compounds for their ability to modulate sodium channel behaviour.

Throughput of test compounds is an important consideration in the choice of screening assay to be used. In some strategies, where hundreds of thousands of compounds are to be tested, it is not desirable to use low throughput means. In other cases, however, low throughput is satisfactory to identify important differences between a limited number of compounds. Often it will be necessary to combine assay types to identify specific sodium channel modulating compounds.

Electrophysiological assays using patch clamp techniques is accepted as a gold standard for detailed characterization of sodium channel compound interactions, and as described in Bean et al., op. cit. and Leuwer, M., et al., op. cit. There is a manual low-throughput screening (LTS) method which can compare 2-10 compounds per day; a recently developed system for automated medium-throughput screening (MTS) at 20-50 patches (i.e. compounds) per day; and a technology from Molecular Devices Corporation (Sunnyvale, CA) which permits automated high-throughput screening (HTS) at 1000-3000 patches (i.e. compounds) per day.

One automated patch-clamp system utilizes planar electrode technology to accelerate the rate of drug discovery. Planar electrodes are capable of achieving high-resistance, cells-attached seals followed by stable, low-noise whole-cell recordings that are comparable to conventional recordings. A suitable instrument is the PatchXpress 7000A (Axon Instruments Inc, Union City, CA). A variety of cell lines and culture techniques, which include adherent cells as well as cells growing spontaneously in suspension are ranked for seal success rate and stability. Immortalized cells (e.g. HEK and CHO) stably expressing high levels of the relevant sodium ion channel can be adapted into high-density suspension cultures.

Other assays can be selected which allow the investigator to identify compounds which block specific states of the channel, such as the open state, closed state or the resting state, or which block transition from open to closed, closed to resting or resting to open. Those skilled in the art are generally familiar with such assays.

Binding assays are also available. Designs include traditional radioactive filter based binding assays or the confocal based fluorescent system available from Evotec OAI group of companies (Hamburg, Germany), both of which are HTS.

Radioactive flux assays can also be used. In this assay, channels are stimulated to open with veratridine or aconitine and held in a stabilized open state with a toxin, and channel blockers are identified by their ability to prevent ion influx. The assay can use radioactive 22[Na] and 14[C] guanidinium ions as tracers. FlashPlate and Cytostar-T plates in living cells avoids separation steps and are suitable for HTS. Scintillation plate technology has also advanced this method to HTS suitability. Because of the functional aspects of the assay, the information content is reasonably good.

Yet another format measures the redistribution of membrane potential using the FLIPR system membrane potential kit (HTS) available from Molecular Dynamics (a division of Amersham Biosciences, Piscataway, NJ). This method is limited to slow membrane potential changes. Some problems may result from the fluorescent background of compounds. Test compounds may also directly influence the fluidity of the cell membrane and lead to an increase in intracellular dye concentrations. Still, because of the functional aspects of the assay, the information content is reasonably good.

Sodium dyes can be used to measure the rate or amount of sodium ion influx through a channel. This type of assay provides a very high information content regarding potential channel blockers. The assay is functional and would measure Na+ influx directly. CoroNa Red, SBFI and/or sodium green (Molecular Probes, Inc. Eugene OR) can be used to measure Na influx; all are Na responsive dyes. They can be used in combination with the FLIPR instrument. The use of these dyes in a screen has not been previously described in the literature. Calcium dyes may also have potential in this format.

In another assay, FRET based voltage sensors are used to measure the ability of a test compound to directly block Na influx. Commercially available HTS systems include the VIPR™ II FRET system (Life Technologies, or Aurora Biosciences Corporation, San Diego, CA, a division of Vertex Pharmaceuticals, Inc.) which may be used in conjunction with FRET dyes, also available from Aurora Biosciences. This assay measures sub-second responses to voltage changes. There is no requirement for a modifier of channel function. The assay measures depolarization and hyperpolarizations, and provides ratiometric outputs for quantification. A somewhat less expensive MTS version of this assay employs the FLEXstation™ (Molecular Devices Corporation) in conjunction with FRET dyes from Aurora Biosciences. Other methods of testing the compounds disclosed herein are also readily known and available to those skilled in the art.

Modulating agents so identified are then tested in a variety of in vivo models so as to determine if they alleviate pain, especially chronic pain or other conditions such as cancer and pruritus (itch) with minimal adverse events. The assays described below in the Biological Assays Section are useful in assessing the biological activity of the instant compounds.

Typically, the efficacy of a compound of the invention is expressed by its IC50 value (“Inhibitory Concentration—50%”), which is the measure of the amount of compound required to achieve 50% inhibition of the activity of the target sodium channel over a specific time period. For example, representative compounds of the present invention have demonstrated IC50's ranging from less than 100 nanomolar to less than 10 micromolar in the patch voltage clamp NaV1.7 electrophysiology assay described herein.

In another aspect of the invention, the compounds of the invention can be used in in vitro or in vivo studies as exemplary agents for comparative purposes to find other compounds also useful in treatment of, or protection from, the various diseases disclosed herein.

Another aspect of the invention relates to inhibiting NaV1.1, NaV1.2, NaV1.3, NaV1.4, NaV1.5, NaV1.6, NaV1.7, NaV1.8, or NaV1.9 activity, preferably NaV1.7 activity, in a biological sample or a mammal, preferably a human, which method comprises administering to the mammal, preferably a human, or contacting said biological sample with a compound as described herein or a pharmaceutical composition comprising a compound as described herein. The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition of NaV1.1, NaV1.2, NaV1.3, NaV1.4, NaV1.5, NaV1.6, NaV1.7, NaV1.8, or NaV1.9 activity in a biological sample is useful for a variety of purposes that are known to one of skill in the art. Examples of such purposes include, but are not limited to, the study of sodium ion channels in biological and pathological phenomena; and the comparative evaluation of new sodium ion channel inhibitors.

The compounds of the invention (or stereoisomers, geometric isomers, tautomers, solvates, metabolites, isotopes, pharmaceutically acceptable salts, or prodrugs thereof) and/or the pharmaceutical compositions described herein which comprise a pharmaceutically acceptable excipient and one or more compounds of the invention, can be used in the preparation of a medicament for the treatment of sodium channel-mediated disease or condition in a mammal.

### Combination Therapies

The compounds of the invention may be usefully combined with one or more other compounds of the invention or one or more other therapeutic agent or as any combination thereof, in the treatment of sodium channel-mediated diseases and conditions. For example, a compound of the invention may be administered simultaneously, sequentially or separately in combination with other therapeutic agents, including, but not limited to:
1. opiates analgesics, e.g., morphine, heroin, cocaine, oxymorphine, levorphanol, levallorphan, oxycodone, codeine, dihydrocodeine, propoxyphene, nalmefene, fentanyl, hydrocodone, hydromorphone, meripidine, methadone, nalorphine, naloxone, naltrexone, buprenorphine, butorphanol, nalbuphine and pentazocine;
2. non-opiate analgesics, e.g., acetomeniphen, salicylates (e.g., aspirin);
3. nonsteroidal antiinflammatory drugs (NSAIDs), e.g., ibuprofen, naproxen, fenoprofen, ketoprofen, celecoxib, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac;
4. anticonvulsants, e.g., carbamazepine, oxcarbazepine, lamotrigine, valproate, topiramate, gabapentin and pregabalin;
5. antidepressants such as tricyclic antidepressants, e.g., amitriptyline, clomipramine, despramine, imipramine and nortriptyline;
6. COX-2 selective inhibitors, e.g., celecoxib, rofecoxib, parecoxib, valdecoxib, deracoxib, etoricoxib, and lumiracoxib;
7. alpha-adrenergics, e.g., doxazosin, tamsulosin, clonidine, guanfacine, dexmetatomidine, modafinil, and 4-amino-6,7-dimethoxy-2-(5-methane sulfonamido-1,2,3,4-tetrahydroisoquinol-2-yl)-5-(2-pyridyl) quinazoline;
8. barbiturate sedatives, e.g., amobarbital, aprobarbital, butabarbital, butabital, mephobarbital, metharbital, methohexital, pentobarbital, phenobartital, secobarbital, talbutal, theamylal and thiopental;
9. tachykinin (NK) antagonist, particularly an NK-3, NK-2 or NK-1 antagonist, e.g., (aR, 9R)-7-[3,5-bis(trifluoromethyl)benzyl)]-8,9,10,11-tetrahydro-9-methyl-5-(4-methylphenyl)-7H-[1,4]diazocino[2,1-g][1,7]-naphthyridine-6-13-dione (TAK-637), 5-[[2R,3S)-2-[(1R)-1-[3,5-bis(trifluoromethylphenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy5-(trifluoromethoxy)phenyl]-methylamino]-2-phenylpiperidine (2S,3S);
10. coal-tar analgesics, in particular paracetamol;
11. serotonin reuptake inhibitors, e.g., paroxetine, sertraline, norfluoxetine (fluoxetine desmethyl metabolite), metabolite demethylsertraline, ‘3 fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine, trazodone and fluoxetine;
12. noradrenaline (norepinephrine) reuptake inhibitors, e.g., maprotiline, lofepramine, mirtazepine, oxaprotiline, fezolamine, tomoxetine, mianserin, buproprion, buproprion metabolite hydroxybuproprion, nomifensine and viloxazine (Vivalan®)), especially a selective noradrenaline reuptake inhibitor such as reboxetine, in particular (S,S)-reboxetine, and venlafaxine duloxetine neuroleptics sedative/anxiolytics;
13. dual serotonin-noradrenaline reuptake inhibitors, such as venlafaxine, venlafaxine metabolite O-desmethylvenlafaxine, clomipramine, clomipramine metabolite desmethylclomipramine, duloxetine, milnacipran and imipramine;
14. acetylcholinesterase inhibitors such as donepezil;
15. 5-HT3 antagonists such as ondansetron;
16. metabotropic glutamate receptor (mGluR) antagonists;
17. local anaesthetic such as mexiletine and lidocaine;
18. corticosteroid such as dexamethasone;
19. antiarrhythimics, e.g., mexiletine and phenytoin;
20. muscarinic antagonists, e.g., tolterodine, propiverine, tropsium chloride, darifenacin, solifenacin, temiverine and ipratropium;
21. cannabinoids;
22. vanilloid receptor agonists (e.g., resinferatoxin) or antagonists (e.g., capsazepine);
23. sedatives, e.g., glutethimide, meprobamate, methaqualone, and dichloralphenazone;
24. anxiolytics such as benzodiazepines, 25. antidepressants such as mirtazapine, 26. topical agents (e.g., lidocaine, capsacin and resiniferotoxin);
27. muscle relaxants such as benzodiazepines, baclofen, carisoprodol, chlorzoxazone, cyclobenzaprine, methocarbamol and orphrenadine;
28. anti-histamines or H1 antagonists;
29. NMDA receptor antagonists;
30. 5-HT receptor agonists/antagonists;
31. PDEV inhibitors;
32. Tramadol®;
33. cholinergic (nicotinc) analgesics;
34. alpha-2-delta ligands;
35. prostaglandin E2 subtype antagonists;
36. leukotriene B4 antagonists;
37. 5-lipoxygenase inhibitors; and
38. 5-HT3 antagonists.

Sodium channel-mediated diseases and conditions that may be treated and/or prevented using such combinations include but not limited to, pain, central and peripherally mediated, acute, chronic, neuropathic as well as other diseases with associated pain and other central nervous disorders such as epilepsy, anxiety, depression and bipolar disease; or cardiovascular disorders such as arrhythmias, atrial fibrillation and ventricular fibrillation; neuromuscular disorders such as restless leg syndrome and muscle paralysis or tetanus; neuroprotection against stroke, neural trauma and multiple sclerosis; and channelopathies such as erythromyalgia and familial rectal pain syndrome.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Computer-Aided Molecular Design Methods

CryoEm structures of NaV1.7 receptors that contain the VSD4 binding site, particularly at better than 3 Angstrom resolution, such as the structure represented in the PDB file (hereinbelow), can be used to carry out computer-based methods of molecular design. The methods herein could also be applied to X-ray crystallographic structures of comparable resolution if obtained.

Computer-based methods of molecular design typically rely on computer programs available and familiar to those skilled in the art of computational chemistry, computer-aided molecular design, molecular modeling, or rational drug design. Such computer programs are designed to be operated on any or all of a desktop workstation, laptop, or super-computer, and/or may utilize processing resources and storage functions commonly referred to as cloud computing. Such programs may utilize any or all of: molecular mechanics (“force field) representations or quantum mechanical calculations of molecular properties. Such programs may permit the serial docking of tens, hundreds, thousands, tens of thousands, hundreds of thousands, or millions of computer-stored molecular structures into a model of the NaV1.7 binding site such as that provided herewith. It is to be understood that “docking” as used herein means obtaining a preferential fit of a given molecular structure, spatially, into the model, based on some scoring function (based on energetic and/or steric criteria), and permitting multiple conformations of a given molecule to be tested.

It is further to be understood that computer-aided design of molecules to fit the binding site may include fitting a scaffold into the binding site and permitting a designer to choose and test representative substitutents on said scaffold for goodness of fit. It is also to be understood that certain computer representations permit the structure of the binding site itself to experience some flexibility such that test inhibitor molecules of varying structure may be tested.

By fits spatially and preferentially is meant that a molecule possesses a 3-dimensional structure, such as obtainable from one of its conformations, that is accommodated geometrically by a cavity or pocket of a protein, such as on the surface or in a solvent accessible cavity.

It is further to be understood that it may not be necessary to use an atomic structural model of an entire receptor, such as the NaV1.7 receptor, for the purpose of identifying additional binding compounds. It may be sufficient to dock molecular structures into a model that comprises a portion of the NaV1.7 receptor that encompasses the ligand binding site. As used herein, the term “portion” or “portion thereof” when referring to the NaV1.7 binding site, is intended to mean the atomic coordinates corresponding to a sufficient number of residues or their atoms that interaction with a compound capable of binding to the site can be accurately described. This can include receptor residues having an atom within about 4.5 Angstrom of a bound compound or a moiety thereof. Particularly useful subsets of the coordinates include, but are not limited to, coordinates of single domains of NaV1.7, in particular the ligand binding domain, coordinates of residues lining an active site such as the ligand binding site, coordinates of residues that participate in important intramolecular, or intermolecular, contacts at an interface, and Ca coordinates. For example, the coordinates of one domain of a protein that contains the active site may be used to design inhibitors that bind to that site, even though the protein is fully described by a larger set of atomic coordinates. Therefore, a set of atomic coordinates that define the entire polypeptide chain of NaV1.7, or the NaV1.7 ligand binding receptor, although useful for many applications, do not necessarily need to be used for the methods described herein.

Structure coordinates for the NaV1.7 receptor or portions thereof according to Appendix 1 may be modified by mathematical manipulation. Such manipulations include, but are not limited to, fractionalization of the raw structure coordinates, additions to, or subtractions from, sets of the raw structure coordinates, by a constant amount inversion, rotation, or reflection the raw structure coordinates, and any combination of the foregoing. Appendix 1 contains coordinates of the VSD4 domain that does not include the channel portion of the receptor.

Those having skill in the art will recognize that atomic structure coordinates are not without error. Thus, it is to be understood that, preferably, any set of structure coordinates obtained for NaV1.7 that have a root mean square deviation (“r.m.s.d.”) of from about 0.5 to about 0.7 Angstrom, or from 0.5 to 0.7 Angstrom, when superimposed, using backbone atoms (N, Ca, C and O), on the structure whose coordinates are found in Appendix 1, are considered to be identical with the structure coordinates listed herein when at least about 50% to 100% of the backbone atoms of NaV1.7 are included in the superposition. Less preferably, a set of structure coordinates obtained for NaV1.7 that have a r.m.s.d. of from about 0.7 to about 1.0 Angstrom, or from 0.7 to 1.0 Angstrom, when superimposed, can be considered to be identical with the structure coordinates listed herein.

Computer-stored molecular structures, or atomic structural information, as used herein, is taken to mean coordinates and identities of atoms found in a molecule or complex, presented or stored in any one of the formats referred to hereinbelow. From atomic structural information it is typically possible to deduce further information important to a chemist, such as the location and type of chemical bonds between atoms in the molecule or complex. It is further to be understood that atomic structural information may be incomplete in the sense that one or more atoms, particularly hydrogen atoms, is missing. However, where there are such missing atoms, it is further to be understood that one of ordinary skill in the art is usually able to deduce the likely position and identity of such atoms, particularly using one or more software programs that would be readily available. The term “atomic model”, or “atomic structural model” may also find use herein. Such terms refer to a set of identities and coordinates for the atoms in a molecule presented in such a way that a 3-dimensional representation of the molecule may be presented to one of skill in the art on, for example, a computer display. Such a 3-dimensional representation may be further manipulated by, for example, rotating or translating it on the display, or by altering its conformation so that the 3-dimensional disposition of its constituent atoms is changed, even though the way in which they are bonded to one another remains unchanged.

All format representations of the atomic structure coordinates described herein may be used according to methods herein. Accordingly, the present invention encompasses the structure coordinates and other information, e.g., amino acid sequence, connectivity tables, vector-based representations, temperature factors, etc., used to generate the three-dimensional structure of the NaV1.7 receptor and its binding site for use in the software programs described herein and other software programs.

While Cartesian coordinates are important and convenient representations of the three-dimensional structure of a protein or polypeptide, those of ordinary skill in the art will readily recognize that other representations of the structure are also useful. Therefore, the three-dimensional structure of a protein, receptor, small organic molecule, or polypeptide, as discussed herein, includes not only the Cartesian coordinate representation, but also all alternative representations of the three-dimensional distribution of atoms. For example, atomic coordinates may be represented as a Z-matrix, wherein a first atom of the molecule is chosen, a second atom is placed at a defined distance from the first atom, a third atom is placed at a defined distance from the second atom so that the first, second and third atoms, when taken in order, make a defined angle. Each subsequent atom is placed at a defined distance from a previously placed atom to make a specified angle with respect to a third atom, and at a specified torsion angle with respect to a fourth atom.

Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the molecule structure. Furthermore, the positions of atoms in a 3-dimensional structure may be represented as fractions of the unit cell (fractional coordinates), or in spherical polar coordinates.

Additional information, such as thermal parameters, which measure the motion of each atom in a crystal structure, chain identifiers, which identify the particular chain of a multi-chain protein in which an atom is located, and connectivity information, which indicates to which atoms a particular atom is bonded, are also useful for representing a 3-dimensional molecular structure.

A variety of data processor programs and formats can be used to store the sequence and structure information on a computer readable medium. Such formats include, but are not limited to, Protein Data Bank (“PDB”) format (Research Collaboratory for Structural Bioinformatics; pdb101.rcsb.org/learn/guide-to-understanding-pdb-data); Structure-data (“SD”) file format (MDL Information Systems, Inc.; Dalby et al., J. Chem. Inf. Comp. Sci. 32:244-255, (1992)), and line-notation, e.g., as used in SMILES (Weininger, D., “SMILES, a Chemical Language and Information System. 1. Introduction to Methodology and Encoding Rules,” J. Chem. Inf. Comp. Sci., 28:31-36, (1988)), and CHUCKLES (Siani, M. A., Weininger, D., Blaney, J., “CHUCKLES: a method for representing and searching peptide and peptoid sequences on both monomer and atomic levels,” J. Chem. Inf. Comp. Sci., 34:588-593, (1994)).

Methods of converting between various formats read by different computer software will be readily apparent to those of ordinary skill in the art, and programs for carrying out such conversions are widely available, either as stand-alone programs, e.g., BABEL (Walters, P. and Stahl, M.,© 1992-1996), available as open-source, or integrated into other software packages.

Ultimately, molecules to be tested for goodness of fit to the NaV1.7 binding site (“test molecules”) have to be quantified for goodness of fit, and preferably selected for testing by means of biochemical assay. Testing may also include synthesizing prior to assaying, in the case of compounds that are not commercially available.

After the 3-dimensional structure of a NaV1.7 receptor, with or without a bound ligand, is determined, the structural information, comprising atomic coordinates, can be stored electronically. Accordingly, the present invention encompasses machine readable media embedded with the three-dimensional structure of the model described herein, or with portions thereof and/or other physicochemical data. By providing a computer readable medium having stored thereon the atomic coordinates of the invention, one of skill in the art can routinely access the atomic coordinates of the invention, or portions thereof, and related information for use in modeling and design programs, as described in detail hereinbelow.

As used herein, “machine readable medium” or “computer readable medium” refers to any media that can be read and accessed directly by a computer or scanner. Such media include, but are not limited to: magnetic storage media, such as floppy discs, hard discs and magnetic tape; optical storage media such as optical discs; CD-ROM, CD-R or CD-RW, and DVD; electronic storage media such as RAM or ROM; and hybrids of these categories such as magnetic/optical storage media. In a preferred embodiment, the information is provided in the form of a machine-readable data storage medium such as a CD-Rom, or on a computer hard-drive. Such media further include paper on which is recorded a representation of the atomic structure coordinates, e.g., Cartesian coordinates, that can be read by a scanning device and converted into a three-dimensional structure with optical character recognition (OCR) technology. The choice of the data storage structure will generally be based on the means chosen to access the stored information.

The machine readable data storage medium can also be used in computational methods of interactive ligand design, specifically the design of synthetic molecules that bind to the NaV1.7 receptor. In one embodiment of the present invention, the structure coordinates of the ligand binding site of NaV1.7 are useful for identifying and/or designing compounds that bind NaV1.7 so that new therapeutic agents may ultimately be developed.

Methods of rational drug design and virtual screening that utilize the coordinates of the proteins of the present invention are preferably performed on one or more computers that comprise at least one central-processing unit for processing machine readable data, coupled via a bus to working memory, a user interface, a network interface, and a machine-readable memory. In preferred embodiments, one or more such computing systems distributed over a computer network are utilized.

In such computing systems: machine-readable memory comprises a data storage material encoded with machine-readable data, wherein the data comprises the structural coordinates of at least one receptor of NaV1.7, with or without a ligand bound thereto; working memory stores an operating system, optionally one or more molecular structure databases, optionally one or more pharmacophores derived from structural coordinates, a graphical user interface and instructions for processing machine-readable data comprising one or more molecular modelling programs such as a deformation energy calculator, a homology modelling tool, a de novo design tool, a “docking tool”, a database search engine, a 2D-3D structure converter and a file format interconverter.

A suitable computer system may be any of the varieties of laptop or desktop personal computer, or workstation, or a networked or mainframe computer or super-computer, that would be available to one of ordinary skill in the art. For example, a suitable computer system may be a personal computer, a workstation, or may be a supercomputer of the type formerly popular in academic computing environments. Computer system may also support multiple processors, in particular GPU processors.

A suitable operating system may be any variety that runs on any of the foregoing computer systems. For example, in one embodiment, operating system 112 is selected from the UNIX family of operating systems. It may also be a LINUX operating system. In another embodiment, operating system 112 is a Windows operating system such as Windows10. In yet another embodiment, operating system 112 is a Macintosh operating system such as MacOS X and later variants, from Apple, Inc.

A graphical user interface (“GUI”) is preferably used for displaying representations of structural coordinates or variations thereof, in 3-dimensional form on user a interface. The GUI also preferably permits the user to manipulate the display of the structure that corresponds to structural coordinates of a NaV1.7 receptor in a number of ways, including, but not limited to: rotations in any of three orthogonal degrees of freedom; translations; projecting the structure on to a 2-dimensional representation; zooming in on specific portions of the structure; coloring of the structure according to a property that varies amongst to different regions of the structure; displaying subsets of the atoms in the structure; coloring the structure by atom type; displaying tertiary structure such as .alpha.-helices and .beta.-sheets as solid or shaded objects; and displaying a surface of a small molecule, peptide, or protein, as might correspond to, for example, a solvent accessible surface, also optionally colored according to some property. Structural coordinates are also optionally copied into computer system memory to facilitate manipulations with one or more of the molecular modelling programs.

A network interface may optionally be used to access one or more molecular structure databases stored in the memory of one or more other computers.

The computational methods of the present invention may be carried out with commercially available programs which run on, or with computer programs that are developed specially for the purpose and implemented on, any of the foregoing computer systems. Commercially available programs typically comprise large integrated molecular modelling packages that contain multiple types of functionality, and are available from vendors such as OpenEye Scientific Software, Inc. (Santa Fe, NM), Chemical Computing Group (Montreal, Canada), and Schrödinger, Inc. (New York, NY).

Alternatively, the computational methods of the present invention may be performed with one or more stand-alone programs each of which carries out one of the functions performed by integrated molecular modelling programs. In particular, certain aspects of the display and visualization of molecular structures may be accomplished by specialized tools).

In still another embodiment, the structure of the NaV1.7 ligand binding site can be used to computationally screen small molecule databases for compounds that can bind in whole, or in part, to NaV1.7. In this screening, the quality of fit of such entities or compounds to the binding site may be judged by methods such as shape complementarity or by estimated interaction energy, according to a number of different methods known to those skilled in the art.

Compounds fitting the NaV1.7 binding site serve as a starting point for an iterative design, synthesis and test cycle in which new compounds are selected and optimized for desired properties including affinity, efficacy, and selectivity with respect to the NaV1.7 binding site and various mutated forms thereof. For example, the compounds can be subjected to additional modification, such as replacement and/or addition of substituents of a core structure identified for a particular class of binding compounds, modeling and/or activity screening if desired, and then subjected to additional rounds of testing.

By “modeling” is meant quantitative and qualitative analysis of molecular structure and/or function based on atomic structural information and interaction models of a receptor and a ligand agonist or antagonist. Modeling thus includes conventional numeric-based molecular dynamic and energy minimization models, interactive computer graphic models, modified molecular mechanics models, distance geometry and other structure-based constraint models. Modeling is preferably performed using a computer and may be further optimized using methods familiar to one of ordinary skill in the art.

Identification of a not previously understood binding conformation to the NaV1.7 binding site has made it possible to apply the principles of molecular modeling to design more compounds that are complementary to the structure of the binding site. Accordingly, computer programs that employ various docking algorithms can be used to identify compounds that fit into the ligand binding domain of NaV1.7. Fragment-based docking can also be used to build molecules inside the NaV1.7 binding site, by placing molecular fragments that have a complementary fit with the site, thereby optimizing intermolecular interactions. Techniques of computational chemistry can also be used to optimize the geometry of the bound conformations.

Docking may be accomplished using commercially available software such as reviewed in: Pagadala N S, Syed K, Tuszynski J. “Software for molecular docking: a review”, Biophys Rev., 2017, 9(2):91-102. Docking is typically followed by energy minimization and molecular dynamics simulations of the docked molecule, using molecular mechanics forcefields. See for example, those reviewed in: Cole, D. J., et al., “The future of force fields in computer-aided drug design”, Future Med. Chem., 11(18), (2019).

Once a compound has been designed or selected by methods such as those described hereinabove, the efficiency with which that compound may bind to the binding site of NaV1.7 may be tested and optimized by computational evaluation. For example, a compound that has been designed or selected to function as an inhibitor (antagonist) of NaV1.7 preferably occupies a volume that does not overlap with the volume occupied by the active site residues. An effective inhibitor of NaV1.7 activity preferably demonstrates a relatively small difference in energy between its bound and free states (i.e., it has a small deformation energy of binding). Thus, the most efficient inhibitors of NaV1.7 should preferably be designed with a deformation energy of binding of not greater than about 10 kcal/mol or, even more preferably, not greater than about 7 kcal/mol. Molecules that bind to NaV1.7 may interact with the receptor in more than one conformation that is similar in overall binding energy. In such cases, the deformation energy of binding is preferably taken to be the difference between the energy of the free compound and the average energy of the conformations observed when the inhibitor binds to the receptor.

A compound selected or designed for binding to NaV1.7 may be further computationally optimized so that in its bound state it would lack repulsive electrostatic interactions with the NaV1.7 structure. Such repulsive electrostatic interactions include non-complementary interactions such as repulsive charge-charge, dipole-dipole and charge-dipole interactions. Specifically, the sum of all electrostatic interactions between the inhibitor and the receptor when the inhibitor is bound to it preferably make a neutral or favorable contribution to the enthalpy of binding.

Specific computer software is available in the art to evaluate compound deformation energy and electrostatic interaction. Examples of programs designed for such uses fall into approximately three levels of sophistication. The crudest level of approximation, molecular mechanics, is also the cheapest to compute and can most usefully be used to calculate deformation energies. Molecular mechanics programs find application for calculations on small organic molecules as well as polypeptides, nucleic acids, proteins, and most other biomolecules.

An intermediate level of sophistication comprises the so-called “semi-empirical” methods, which are relatively inexpensive to compute and are most frequently employed for calculating deformation energies of organic molecules. Examples of program packages that provide semi-empirical capability are reviewed in: Christensen, A. S., et al., Chem. Rev., 2016, 116, 9, 5301-5337.

The highest level of sophistication is achieved by those programs that employ so-called ab initio quantum chemical methods and methods of density functional theory, for example those reviewed in: Kulik, et al., J. Phys. Chem. B, 2012, 116, 41, 12501-12509. These programs may be installed, for instance, on a computer workstation, as is well-known in the art. Other hardware systems and software packages will be known to those skilled in the art.

In general, databases of small molecules can be computationally screened to identify molecules that are likely to bind in whole, or in part, to a NaV1.7 binding site of interest. In such screening, the quality of fit of molecules to the binding site may be judged by any of a number of methods that are familiar to one of ordinary skill in the art, including shape complementarity or by estimated interaction energy Such methods are preferably applicable to ranking compounds for their ability to binding to the NaV1.7 receptor.

In a preferred method, potential binding compounds may be obtained by rapid computational screening. Such a screening comprises testing a large number, which may be hundreds, or may preferably be thousands, or more preferably tens of thousands, or even more preferably hundreds of thousands of molecules whose formulae are known and for which at least one conformation can be readily computed.

The databases of small molecules include any virtual or physical database, such as electronic and physical compound library databases. Preferably, the molecules are obtained from one or more molecular structure databases that are available in electronic form and any proprietary database of compounds with known medicinal properties, as is found in a large or small pharmaceutical company.

The molecules in such databases for use with the present invention are preferably stored as a connection table, with or without a 2D representation that comprises coordinates in just 2 dimensions, say x and y, for facilitating visualization on a computer display. The molecules are more preferably stored as at least one set of 3D coordinates corresponding to an experimentally derived or computer-generated molecular conformation. If the molecules are only stored as a connection table or a 2D set of coordinates, then it can be necessary to generate a 3D structure for each molecule before proceeding with a computational screen, for example, if the molecules are to be docked into a receptor structure during screening. Programs for converting 2D molecular structures or molecule connection tables to 3D structures are available to those skilled in the art.

As part of a computational screen, it is possible to “dock” 3D structures of molecules from a database into the NaV1.7 binding site on a high throughput basis. Such a procedure can normally be subject to a number of user-defined parameters and thresholds according to desired speed of throughput and accuracy of result. Such parameters include the number of different starting positions from which to start a docking simulation and the number of energy calculations to carry out before rejecting or accepting a docked structure. Such parameters and their choices are familiar to one of ordinary skill in the art. Structures from the database can be selected for synthesis to test their ability to bind NaV1.7 if their docked energy is below a certain threshold. Methods of docking are further described elsewhere herein.

Alternatively, it is possible to carry out a “molecular similarity” search for molecules that are potential inhibitors of NaV1.7. If a pharmacophore has been developed from a knowledge of the NaV1.7 binding site, then molecules whose structures map on to that pharmacophore are to be found. A pharmacophore defines a set of contact sites on the surface of the binding site, accompanied by the distances between them. A similarity search attempts to find molecules in a database that have at least one favorable 3D conformation whose structure overlaps favorably with the pharmacophore. For example, a pharmacophore may comprise a lipophilic pocket at a particular position, a hydrogen-bond acceptor site at another position and a hydrogen bond donor site at yet another specified position accompanied by distance ranges between them. A molecule that could potentially fit into the active site is one that can adopt a conformation in which a H-bond donor in the active site can reach the H-bond acceptor site on the pharmacophore, a H-bond acceptor in the active site can simultaneously reach the H-bond donor site of the pharmacophore and, for example, a group such as a phenyl ring can orient itself into the lipophilic pocket.

Even where a pharmacophore has not been developed, molecular similarity principles may be employed in a database searching regime (see, for example, Johnson, M. A.; Maggiora, G. M., Eds. Concepts and Applications of Molecular Similarity, New York: John Wiley & Sons (1990)) if at least one molecule that fits well into the NaV1.7 binding site is known. In a preferred embodiment, it is possible to search for molecules that have certain properties in common with those of the molecule(s) known to bind. For example, such properties include numbers of hydrogen bond donors or numbers of hydrogen bond acceptors, or overall hydrophobicity within a particular range of values. Alternatively, even where a pharmacophore is not known, similar molecules may be selected on the basis of optimizing an overlap criterion with the molecule of interest. For example, where the structures of test molecules that bind are known, a model of the test molecule may be superimposed over the model of the NaV1.7 structure.

In searching a molecular structure database, a specialized database searching tool that permits searching molecular structures and sub-structures is typically employed, as is familiar to one of skill in the art.

Molecules that bind to the NaV1.7 binding site can be designed by a number of methods, including: exploiting available structural and functional information; by deriving a quantitative structure-activity relationship (QSAR); and by using a combination of such information to design new compound libraries. In particular, focused libraries having molecular diversity at one or more particular groups attached to a core structure or scaffold, may be used. Preferably, structural data is incorporated into the iterative design process. For example, one of skill in the art may use one of several methods to screen molecules or fragments for their ability to associate with the NaV1.7 binding site. This process may begin with visual inspection of, for example, the NaV1.7 binding site on a computer screen. Selected fragments or chemical entities may then be positioned into the site, or a portion thereof. Docking may be accomplished using computer software as described hereinabove, followed by energy minimization and molecular dynamics with standard molecular mechanics force-fields, as also described hereinabove.

The design of molecules that bind to NaV1.7 generally involves consideration of two factors. The molecule must be capable of first physically, and second structurally, associating with NaV1.7. The physical interactions underpinning this association can be covalent or non-covalent. For example, covalent interactions may be important for designing irreversible or “suicide” inhibitors of a protein. Non-covalent molecular interactions that are important in the association of NaV1.7 with molecules that bind to it include hydrogen bonding, ionic, van der Waals, and hydrophobic interactions. Structurally, the compound must be able to assume a conformation that allows it to associate with the binding site of NaV1.7. Although certain portions of the compound will not necessarily directly participate in this association with NaV1.7, those portions may still influence the overall conformation of the molecule. This, in turn, may have a significant impact on potency. Such conformational requirements include the overall three-dimensional structure and orientation of a functional group or moleculein relation to all or a portion of the binding site, or the spacing between functional groups of a compound comprising several functional groups that directly interact with NaV1.7.

In general, the potential binding effect of a compound on NaV1.7 may be analyzed prior to its actual synthesis and testing by the use of computer modeling techniques. If the theoretical structure of the given compound suggests insufficient interaction and association between it and the NaV1.7 binding site, synthesis and testing of the compound need not be carried out. However, if computer modeling indicates a strong interaction, the molecule may then be synthesized and tested for its ability to bind to the NaV1.7 binding site and thereby inhibit its activity. In this manner, costly synthesis of ineffective compounds may be avoided.

Among the computational techniques that enable the rational design of molecules that bind to NaV1.7, it is key to have access to visualization tools, programs for calculating properties of molecules, and programs for fitting ligand structures into three-dimensional representations of the receptor binding site. Computer program packages for facilitating each of these capabilities have been referred to herein, and are available to one of ordinary skill in the art. Visualization of molecular properties, such as field properties that vary through space, can also be particularly important and may be aided by computer programs familiar to those of skill in the art.

A molecular property of particular interest when assessing suitability of drug compounds is its hydrophobicity. An accepted and widespread measure of hydrophobicity is LogP, the log₁₀ of the octanol-water partition coefficient. It is customary to use the value of LogP for a designed molecule to assess whether the molecule could be suitable for transport across a cell membrane, if it were to be administered as a drug. Measured values of LogP are available for many compounds. Methods and programs for calculating LogP are also available, and are particularly useful for molecules that have not been synthesized or for which no experimental value of LogP is available.

Once an NaV1.7-binding compound has been optimally selected or designed, as described hereinabove, substitutions may then be made in some of its atoms or chemical groups in order to improve or modify its binding properties. Generally, initial substitutions are conservative, i.e., the replacement group will have approximately the same size, shape, hydrophobicity, polarity and charge as the original group. For selection of appropriate groups, any of several chemical models can be used, e.g., isolobal or isosteric analogies. Groups known to be bio-isosteres of one another are particularly preferred. One of skill in the art will understand that substitutions known in the art to alter conformation are preferably avoided. Such altered chemical compounds may then be analyzed for efficiency of binding to NaV1.7 by the same computer methods described hereinabove.

Suitable test compounds can be designed, as is further described herein, or can be obtained from a library of compounds, and include, by means of illustration and not limitation, small organic molecules, peptides and peptidomimetics. A library of compounds may be a combinatorial library, generated either in the laboratory, or virtually in a computer, or may be a computer-encoded library of molecules that are commercially available from one or more vendors. The library of compounds may further be a commercially available selection of molecules that has been selected for a particular property, or for representative diversity of properties.

In some embodiments, the atomic coordinates of a compound that fits into the NaV1.7 binding site also can be used for modeling to identify compounds or fragments that bind the site. Thus, the present invention also provides for a computational method that uses three dimensional models of a NaV1.7 receptor, such as derived from CryoEM, preferably containing coordinates of a bound molecule. Such models can be said to be experimentally derived, as opposed to derived computationally, such as by homology modeling. Generally, the computational method of designing a NaV1.7 receptor ligand involves determining which amino acid or amino acid residues of the NaV1.7 receptor binding site interact with at least one moiety (“first moiety”) of the ligand, by using a three dimensional model that comprises the NaV1.7 receptor binding site. The method further comprises selecting at least one chemical modification of the first moiety to produce a second moiety that either decreases or increases an interaction between the interacting amino acid residue and the second moiety when compared to the interaction between the interacting amino acid residue and the original moiety. Such a modification can be carried out virtually, by using a computer modeling program as further described herein, or in the laboratory, as applied to a sample of the molecule or by synthesizing an analog that differs from the initial molecule by such a modification.

Computational methods may further comprise quantifying a change in interaction between the interacting amino acid in the NaV1.7 binding site and the ligand after modification of the first moiety. The modification can either enhance or reduce a hydrogen bonding interaction, a charge interaction, a hydrophobic interaction, a van der Waals interaction, or a dipole interaction between the second moiety and the interacting amino acid, as compared to the interaction between the first moiety and the interacting amino acid. Chemical modifications will often enhance or reduce interactions between an atom of a NaV1.7 binding site amino acid and an atom of a ligand.

### NaV1.7 Binding Modes

Voltage-gated sodium channels have been shown to have four (4) discrete voltage sensing domains (VSD). VSD4 has been identified as the most promising target for molecular design, and comprises 4 helices, S1-S4.

Inhibitors of the NaV1.7 VSD4 binding domain have been identified previously as falling within two chemical classes: aryl-sulfonamides (such as GNE-616, J. Med. Chem., 62, 4091 (2019), and acyl sulfonamides (such as GDC-0276, J. Med. Chem., 64, 2953 (2021)).

Hitherto, only one small molecule co-crystal structure of an inhibitor (GX-936) bound to the NaV1.7 VSD4 domain has been published (Science, (2015), 350 (6267), aac5464). In this structure, one key interaction is between an anionic “warhead” on the ligand and two arginine residues in the receptor binding site, and a second is an aryl/CF3 interaction between phCF3 on the ligand and a pi-stacking relationship with residue Y1537 in the binding pocket.

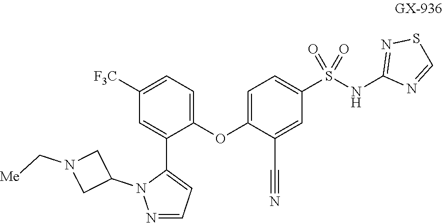 [SMILES: CCN1CC(n2nccc2-c2cc(C(F)(F)F)ccc2Oc2ccc(S(=O)(=O)Nc3ncsn3)cc2C#N)C1]

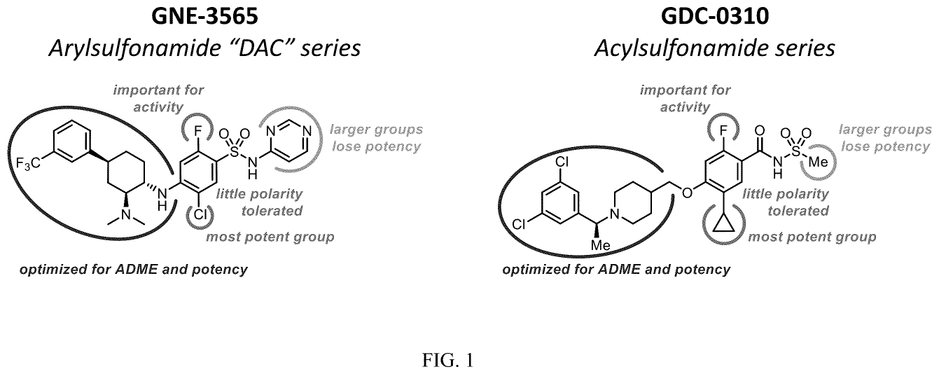

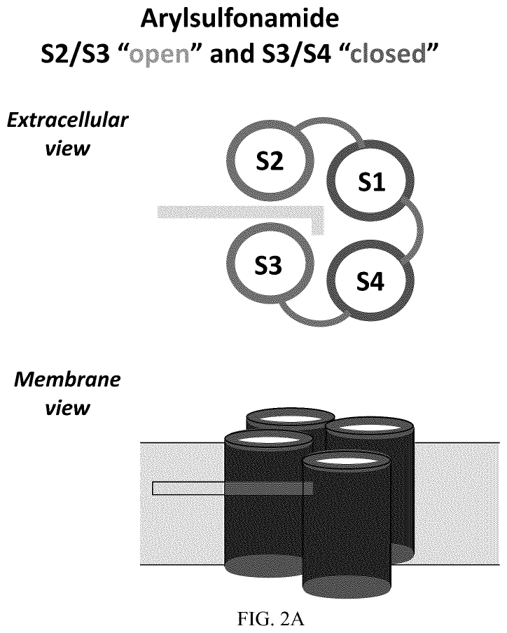

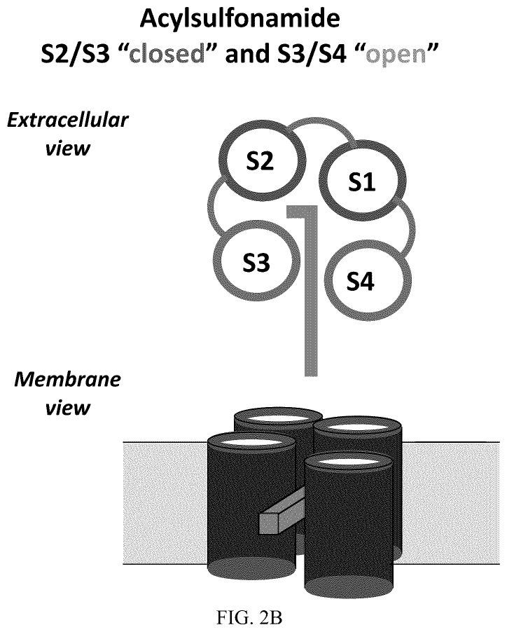

Conversely, efforts to determine co-crystal structures of acyl sulfonamide ligands bound to NaV1.7 have been unsuccessful, meaning that attempts at molecular design has been in some sense, “blind”, and relying on structure-activity relationships (SAR) and assumptions as to the respective binding modes of both series. As shown in FIG. 1, the two classes of compounds exhibited “pseudoparallel” SAR trends while apparently demonstrating competitive binding. Proposals to reconcile the different SAR while rationalizing their binding poses were unsatisfactory (see, e.g., J. Med. Chem. 62, 908 (2019)). FIGS. 2A and 2B show schematics of proposed binding poses of aryl- and acyl-sulfonamide compounds of the prior art.

By using cryo-EM, however, as described elsewhere herein, it was possible to independently confirm the binding posture of the aryl-sulfonamide series of compounds while, for the first time revealing that the acyl-sulfonamide compounds bind to the VSD4 domain in a different manner, with accompanying changes in protein conformation.

Specifically, it becomes clear that aryl sulfonamides bind between helices S2 and S3, pushing residue Tyr1537 “upwards”; the helices S3/S4 form a “wall” abutting the anionic “warhead”. Conversely, acyl sulfonamides bind to a different pocket of the VSD4 domain, wherein the binding site is between helices S3 and S4, the Tyr1537 residue is pointed “down” into intrahelical space between S2 and S3.

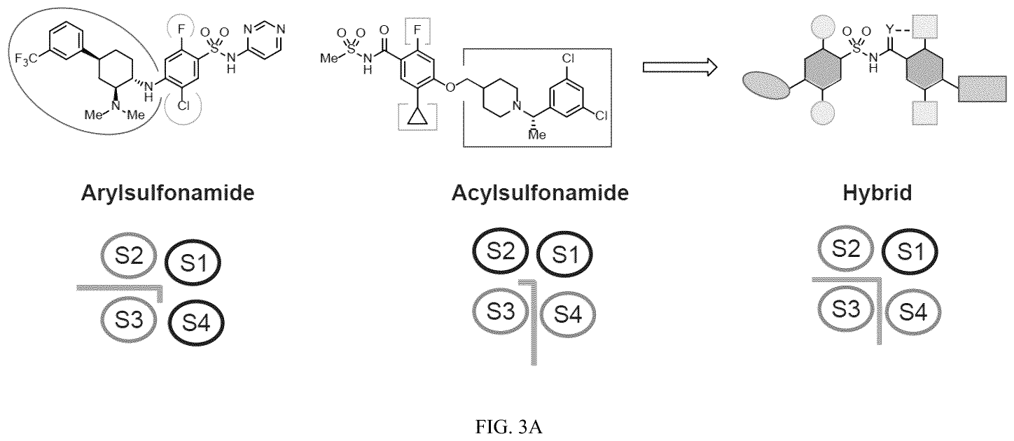

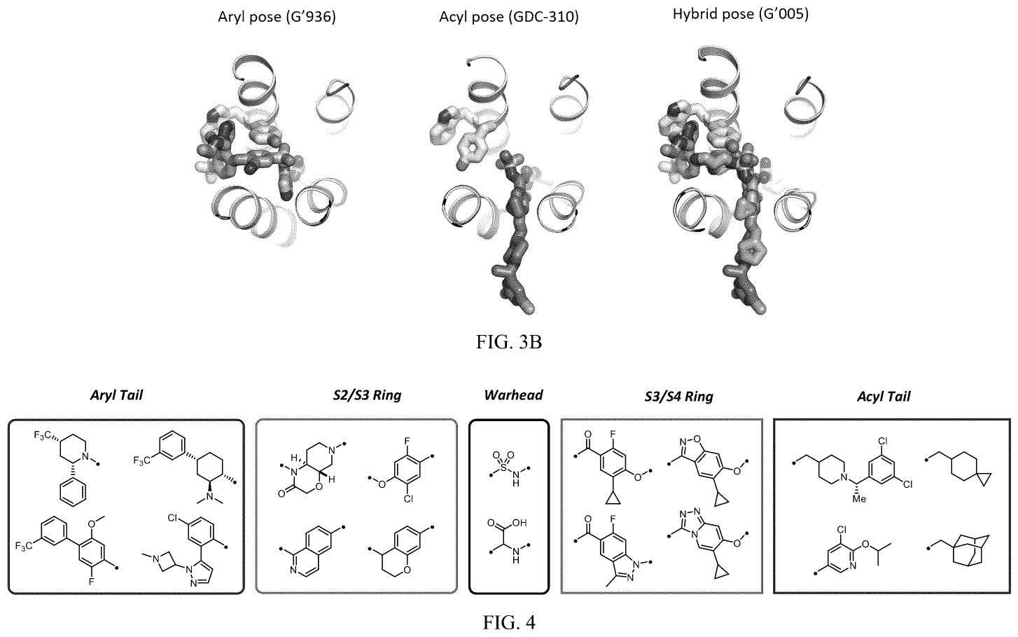

This is summarized in FIGS. 3A, 3B. Other facets of the differentiated binding poses between the two classes of compounds are as follows: Aryl sulfonamides interact with receptor residues R1602 and R1608; Acyl sulfonamides interact with receptor residues R1605 and R1608. Furthermore, in the bound aryl sulfonamide configurations, S3/S4 pocket interactions appear to be largely lipophlic space filling. Conversely, for bound acyl sulfonamides, the ligands' “tail” reside wholly in the plasma membrane that surrounds the receptor.

The understanding of two distinct binding poses leads to the possibility that molecules could be designed that simultaneously bind both pockets, thereby relying less on membrane interactions for potency, and more for an increased interaction with a larger and more complex binding pocket. The principle behind devising such “hybrid” molecules is illustrated in FIG. 4.

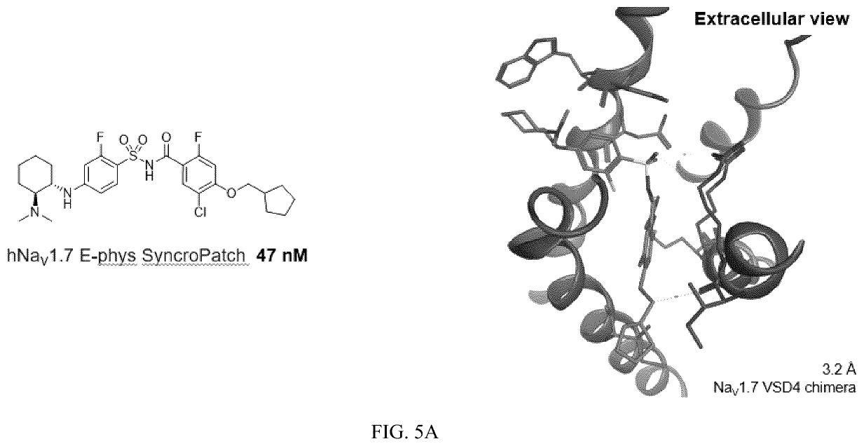

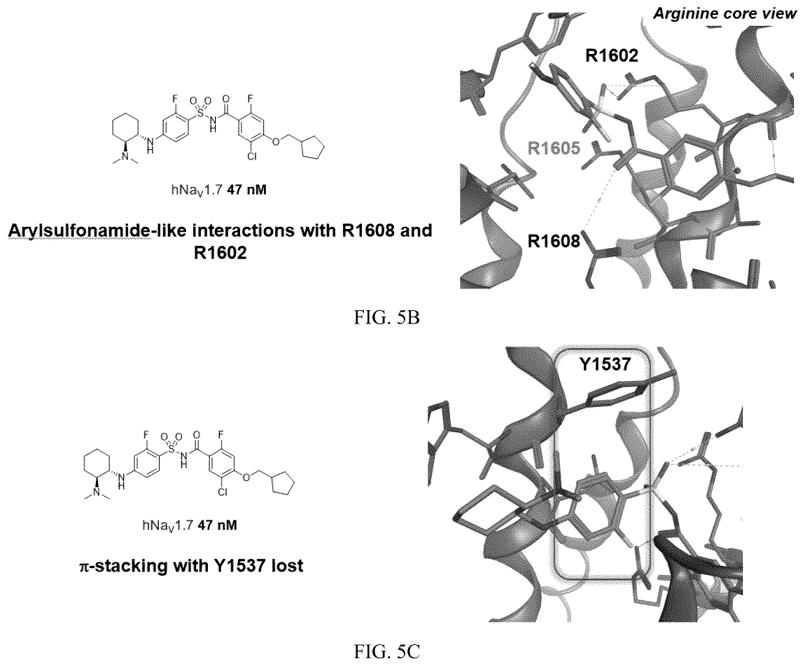

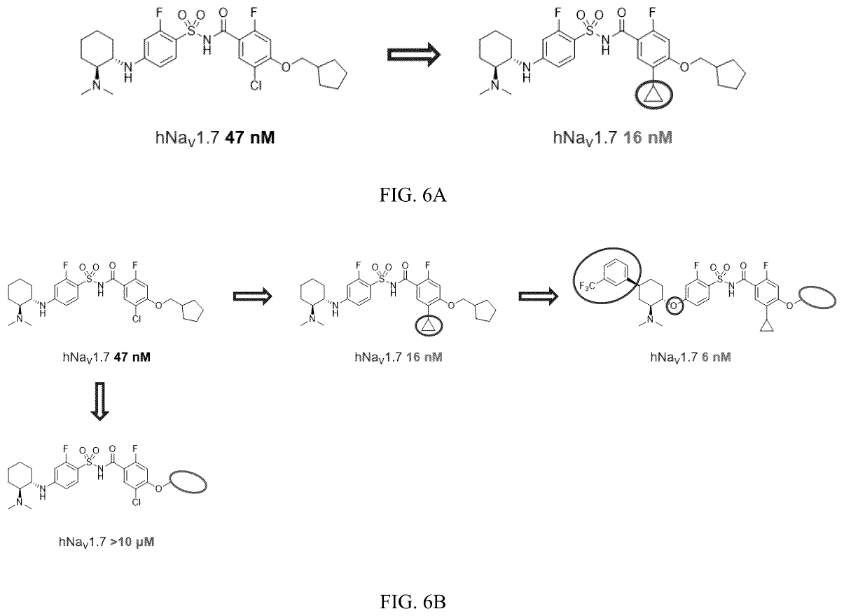

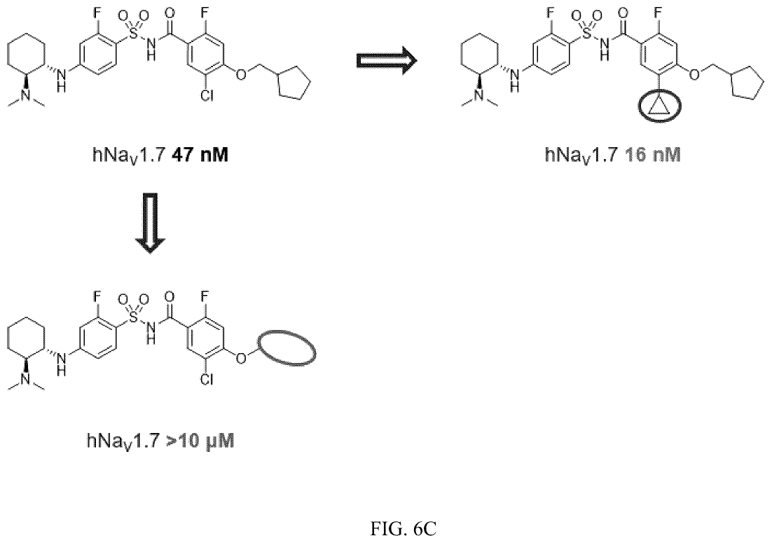

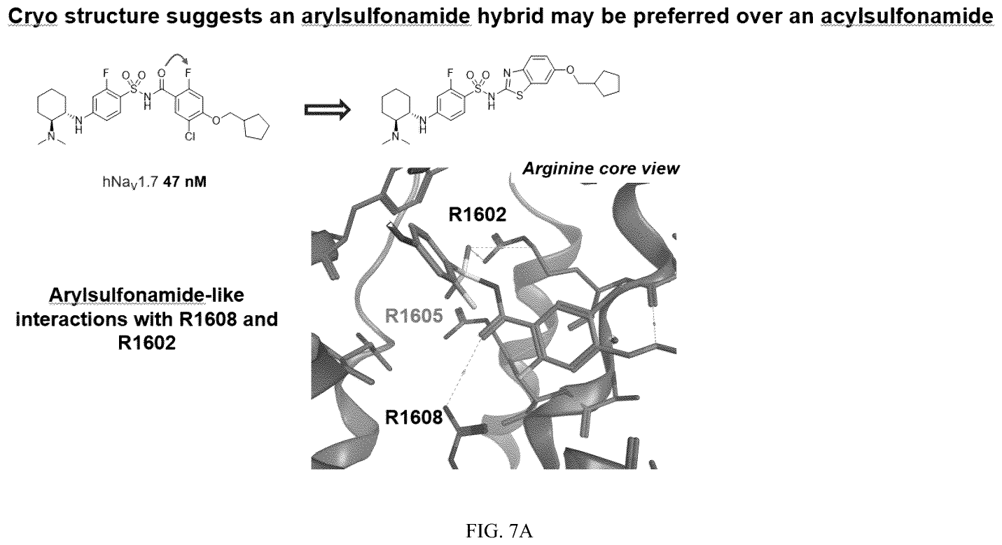

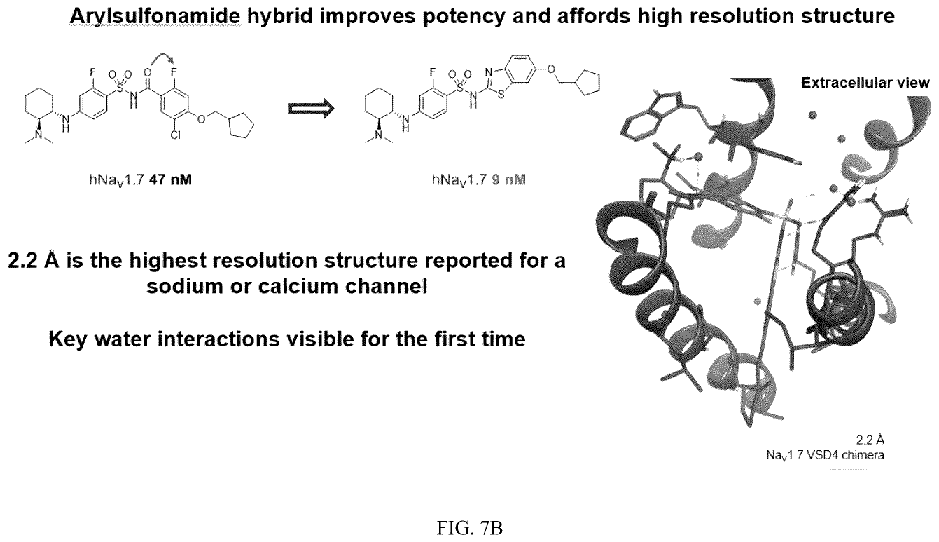

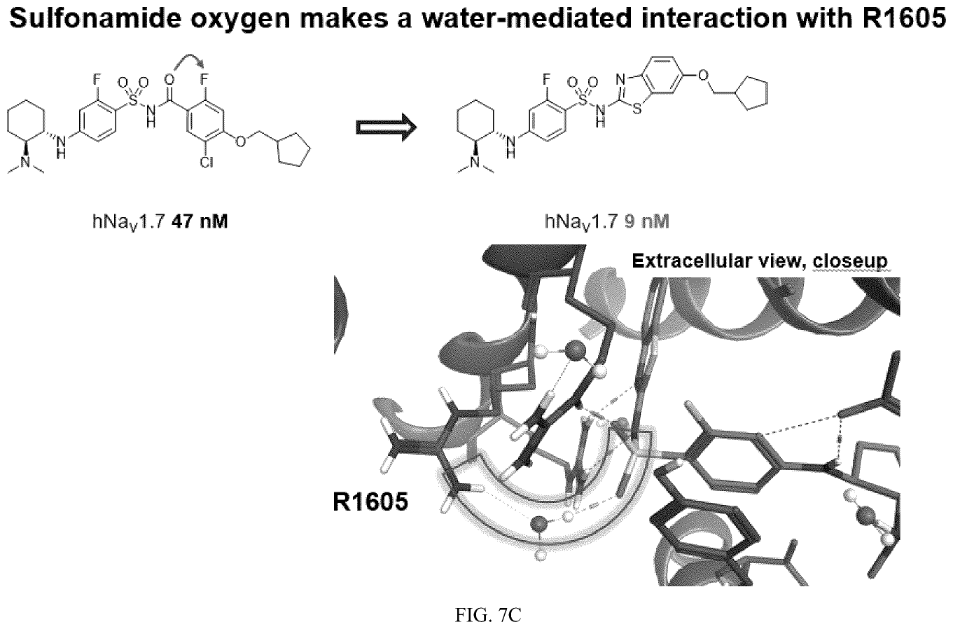

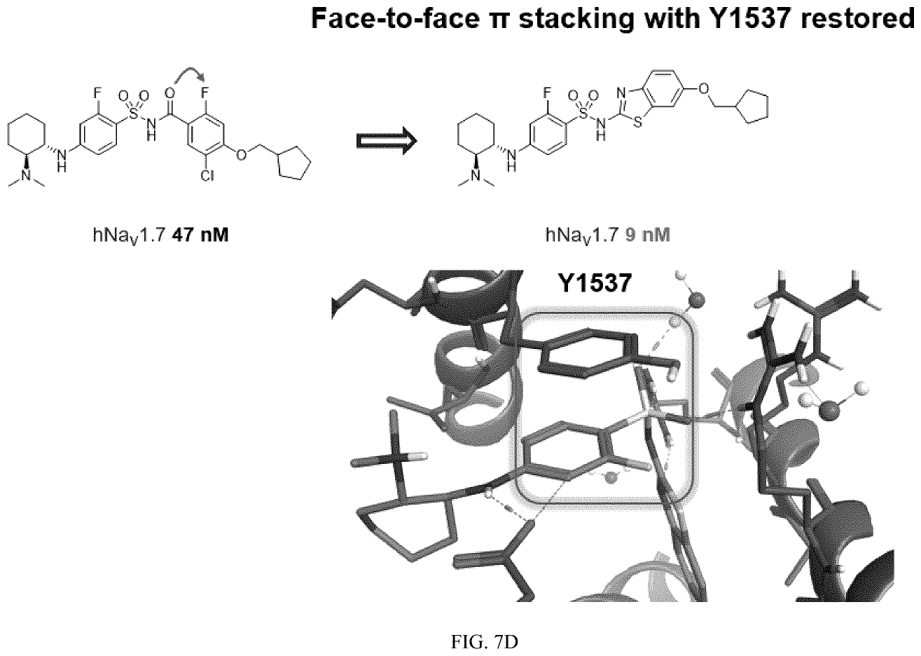

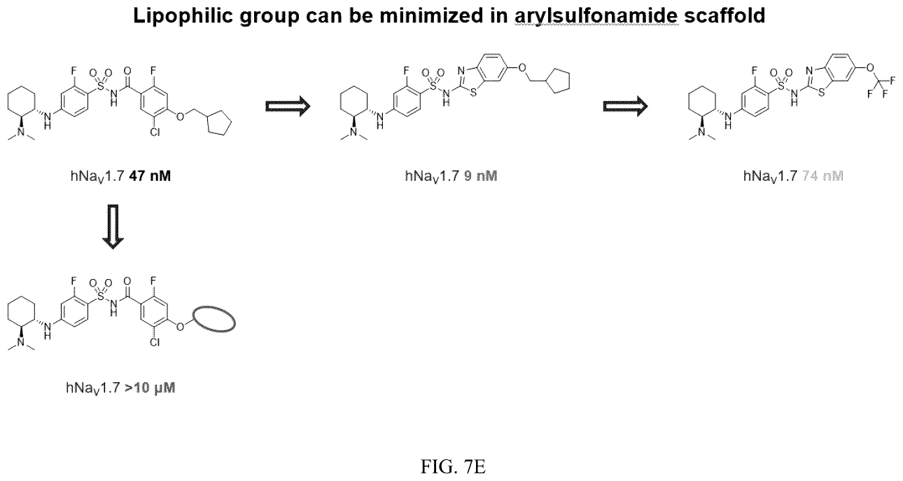

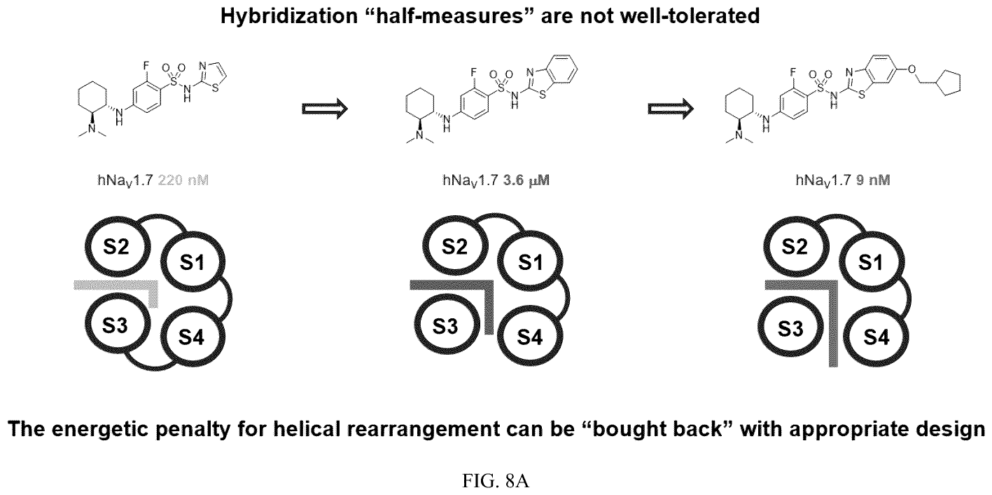

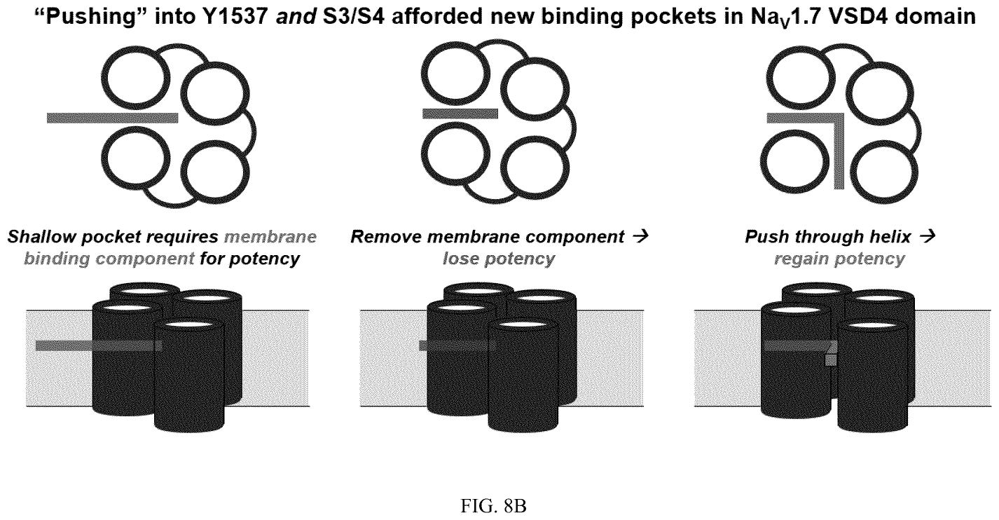

Various aspects of design and properties of “hybrid” molecules that bind NaV1.7 are shown in FIGS. 4, 5A-5C, 6A-6C, 7A-7E, 8A and 8B. Such features can be utilized to rationalize the binding properties of molecules exemplified herein as well as to design further molecules consistent with the instant invention.

The invention will be more fully understood by reference to the following examples. They should not, however, be construed as limiting the scope of the invention.

### EXAMPLES

The following examples serve to provide guidance to a skilled artisan to prepare and use the compounds, compositions and methods of the invention. While a particular embodiment of the present invention are described, the skilled artisan will appreciate that various changes and modifications can be made without departing from the spirit and scope of the inventions. Furthermore, while the Examples are not presented in a continuous sequence, the numbers are unique. Gaps in the numbering sequence are not indicative of omitted material (either intentionally or unintentionally), and the assignment of a particular number to a particular compound or example is arbitrary and without significance.

The chemical reactions in the examples described can be readily adapted to prepare a number of other compounds of the invention, and alternative methods for preparing the compounds of this invention are deemed to be within the scope of this invention. For example, the synthesis of non-exemplified compounds according to the invention can be successfully performed by modifications apparent to those skilled in the art, for example, by appropriately protecting interfering groups by utilizing other suitable reagents known in the art other than those described, and/or by making routine modifications of reaction conditions.

In the examples below, unless otherwise indicated all temperatures are set forth in degrees Celsius. Commercially available reagents were purchased from suppliers such as Aldrich Chemical Company, Lancaster, TCI or Maybridge and were used without further purification unless otherwise indicated. The reactions set forth below were done under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried. ¹H NMR spectra were obtained in deuterated CDCl₃, d₆-DMSO, CH₃OD or d₆-acetone solvent solutions (reported in ppm) using or trimethylsilane (TMS) or residual non-deuterated solvent peaks as the reference standard. When peak multiplicities are reported, the following abbreviates are used: s (singlet), d (doublet), t (triplet), q (quartet), m (multiplet), br (broad), dd (doublet of doublets), dt (doublet of triplets). Coupling constants, when given, are reported in Hz (Hertz).

Although the preparation of the free-base or a specific salt form may be illustrated in the Examples herein, it is understood that the free-base and its acid or base salt forms can be interconverted using standard techniques

All abbreviations used to describe reagents, reaction conditions or equipment are intended to be consistent with the definitions set forth in the “List of standard abbreviates and acronyms”. The chemical names of discrete compounds of the invention were obtained using the structure naming feature of ChemDraw naming program. Structures in the Examples herein are shown with a formula ((I), (II), etc., as described elsewhere herein) that each respective compound falls within.

### Abbreviations

MeCN Acetonitrile
EtOAc Ethyl acetate
DCM Dichloromethane
DIPEA Diisopropylethylamine
DEA Diethylamine
DMAP 4-dimethylaminopyridine
DMF N,N-Dimethylformamide
DMSO Dimethyl sulfoxide
FA Formic acid
IPA Isopropyl alcohol
TFA Trifluoroacetic acid
EDCI 1-Ethyl-3-(3-dimethylaminopropyl)carbodiimide hydrochloride
HCl Hydrochloric acid
HPLC High Pressure Liquid Chromatography
LCMS Liquid Chromatography Mass Spectrometry
MeOH Methanol
NMP N-methyl-2-pyrrolidone
RT Retention time
SFC Supercritical Fluid Chromatography
THF Tetrahydrofuran
TEA Triethylamine

### Example 1: 5-chloro-4-(cyclopentylmethoxy)-2-fluoro-N-((4-((1-methylazetidin-3-yl)oxy)piperidin-1-yl)sulfonyl)benzamide

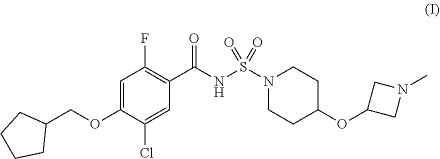 [SMILES: CN1CC(OC2CCN(S(=O)(=O)NC(=O)c3cc(Cl)c(OCC4CCCC4)cc3F)CC2)C1]

### Step 1: tert-butyl 3-(pyridin-4-yloxy)azetidine-1-carboxylate

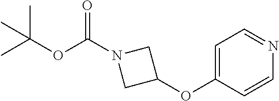 [SMILES: CC(C)(C)OC(=O)N1CC(Oc2ccncc2)C1]

To a solution of 4-hydroxypyridine (6.2 g, 65.19 mmol) in THF (190 ml) was added PPh₃ (21.37 g, 81.49 mmol), 1-boc-3-hydroxyazetidine (14.12 g, 81.49 mmol) and DIAD (16.16 ml, 81.49 mmol) at room temperature. The mixture was stirred at 70° C. for 16 h. After cooling to room temperature, the reaction was concentrated in vacuo. The reaction mixture was dissolved in 1.0 M aqueous HCl solution (100 mL) and extracted with DCM (100 mL). The aqueous lawyer was adjusted to pH=12 using 1.0 M aqueous NaOH and then extracted with DCM (100 mL×3). The combined organic layers were washed with brine (100 mL), dried over anhydrous Na₂SO₄, filtered and concentrated in vacuo. The crude residue was purified by silica gel chromatography (solvent gradient: 0-50% EtOAc in petroleum ether) to afford the title compound (6.11 g, 75%) as colorless oil. LCMS (ESI) m/z: 251.1 [M+H]⁺.

### Step 2: tert-butyl 3-(piperidin-4-yloxy)azetidine-1-carboxylate

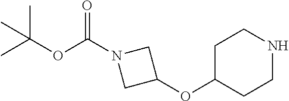 [SMILES: CC(C)(C)OC(=O)N1CC(OC2CCNCC2)C1]

To a solution of PtO₂ (5.47 g, 24.09 mmol) in EtOH (150 mL) was added tert-butyl 3-(pyridin-4-yloxy)azetidine-1-carboxylate (6.7 g, 26.77 mmol) and TsOH (5.07 g, 29.45 mmol). The mixture was stirred at 45° C. for 48 h under hydrogen atmosphere (45 psi). After cooling to room temperature, the mixture was added 1 M NaOH aqueous solution (200 mL) and stirred for 10 min, then the mixture was filtered, concentrated in vacuo to remove most EtOH and extracted with DCM (100 mL×4). The combined organic layers were washed with brine (100 mL×2), dried over anhydrous Na₂SO₄, filtered, and concentrated in vacuo. The residue was purified by silica gel chromatography (solvent gradient: 0-10% MeOH in DCM (1% NH₃·H₂O)) to afford the title compound (2.5 g, 36%) as yellow oil. ¹H NMR (400 MHz, CDCl₃) δ 4.36-4.28 (m, 1H), 4.12-4.04 (m, 2H), 3.86-3.77 (m, 2H), 3.47-3.41 (m, 1H), 3.18-3.12 (m, 2H), 2.82-2.71 (m, 2H), 1.99-1.88 (m, 2H), 1.65-1.52 (m, 2H), 1.44 (s, 9H).

### Step 3: tert-butyl 3-((1-sulfamoylpiperidin-4-yl)oxy)azetidine-1-carboxylate

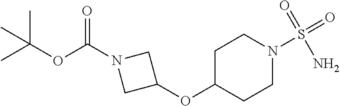 [SMILES: CC(C)(C)OC(=O)N1CC(OC2CCN(S(N)(=O)=O)CC2)C1]

To a mixture of tert-butyl 3-(piperidin-4-yloxy)azetidine-1-carboxylate (500 mg, 1.95 mmol) in 1,4-dioxane (20 mL) was added sulfamide (469 mg, 4.88 mmol). The resulting mixture was stirred at 110° C. for 16 h under nitrogen atmosphere. After cooling to room temperature, the reaction was added water (20 mL), extracted with EtOAc (20 mL×3). The combined organic layers were washed with brine (20 mL×3), dried over anhydrous Na₂SO₄, filtered and concentrated in vacuo to afford the title compound (600 mg, crude) as a yellow solid that required no further purification. ¹H NMR (400 MHz, CDCl₃) δ 4.35-4.27 (m, 1H), 4.15-4.10 (m, 2H), 3.86-3.79 (m, 2H), 3.55-3.45 (m, 1H), 3.45-3.30 (m, 2H), 3.15-3.00 (m, 2H), 1.94-1.83 (m, 2H), 1.79-1.68 (m, 2H), 1.44 (s, 9H).

### Step 4: tert-butyl 3-((1-(N-(5-chloro-4-(cyclopentylmethoxy)-2-fluorobenzoyl)sulfamoyl)piperidin-4-yl)oxy)azetidine-1-carboxylate

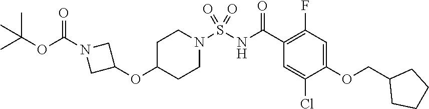 [SMILES: CC(C)(C)OC(=O)N1CC(OC2CCN(S(=O)(=O)NC(=O)c3cc(Cl)c(OCC4CCCC4)cc3F)CC2)C1]

To a mixture of DMAP (0.63 g, 5.13 mmol) and EDCI (0.59 g, 3.08 mmol) in DCM (35 mL) was added 5-chloro-4-(cyclopentylmethoxy)-2-fluoro-benzoic acid (0.7 g, 2.57 mmol) and tert-butyl 3-[(1-sulfamoyl-4-piperidyl)oxy]azetidine-1-carboxylate (0.86 g, 2.57 mmol). The resulting mixture was stirred at room temperature for 16 h under nitrogen atmosphere. The reaction was quenched with 10% citric aqueous solution (50 mL) and extracted with DCM (50 mL×3). The combined organic layers were washed with brine (100 mL), dried over anhydrous Na₂SO₄, filtered and concentrated in vacuo. The crude residue was purified by silica gel chromatography (solvent gradient: 0-7% MeOH in DCM) to afford the title compound (1.2 g, 79%) as a white solid. LCMS (ESI) m/z: 490.2 [M-100+H]⁺.

### Step 5: N-((4-(azetidin-3-yloxy)piperidin-1-yl)sulfonyl)-5-chloro-4-(cyclopentylmethoxy)-2-fluorobenzamide

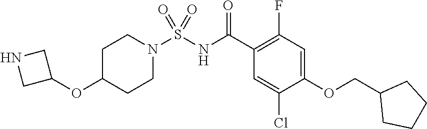 [SMILES: O=C(NS(=O)(=O)N1CCC(OC2CNC2)CC1)c1cc(Cl)c(OCC2CCCC2)cc1F]

To a solution of 2,2,2-trifluoroacetic acid (1.3 mL, 17.31 mmol) in DCM (17 mL) was added tert-butyl 3-[[1-[[5-chloro-4-(cyclopentylmethoxy)-2-fluoro-benzoyl]sulfamoyl]-4-piperidyl]oxy]azetidine-1-carboxylate (1.2 g, 2.03 mmol) at room temperature. The mixture was stirred at room temperature for 1 h. The mixture was concentrated in vacuo, then the residue was dissolved in DCM (30 mL), washed with 10% NaOH aqueous solution (10 mL) and brine (30 mL), dried over anhydrous Na₂SO₄, filtered and concentrated in vacuo to afford the title compound (0.90 g, crude) as a white solid that required no further purification. LCMS (ESI) m/z: 490.1 [M+H]⁺.

### Step 6: 5-chloro-4-(cyclopentylmethoxy)-2-fluoro-N-((4-((1-methylazetidin-3-yl)oxy)piperidin-1-yl)sulfonyl)benzamide

To a mixture of N-((4-(azetidin-3-yloxy)piperidin-1-yl)sulfonyl)-5-chloro-4-(cyclopentylmethoxy)-2-fluorobenzamide (200 mg, 0.41 mmol) in DCM (6 mL) was added formaldehyde (0.82 mL, 10.85 mmol, 37% in water) and NaBH(OAc)₃ (432 mg, 2.04 mmol). The resulting mixture was stirred at room temperature for 16 h. The mixture was quenched with saturated aqueous NaHCO₃ solution (20 mL) to pH>7 and then extracted with DCM (30 mL×2). The combined organic layers were washed with brine (25 mL), then dried over anhydrous Na₂SO₄, filtered and concentrated in vacuo. The crude residue was purified by reverse phase chromatography (solvent gradient: acetonitrile 30-60%/(0.2% HCOOH) in water) to afford the title compound (15.48 mg, 7%) as a white solid. ¹H NMR (400 MHz, DMSO-d₆) δ 7.71 (d, J=7.6 Hz, 1H), 6.96 (d, J=12.4 Hz, 1H), 4.43-4.36 (m, 1H), 4.25-4.17 (m, 2H), 3.95 (d, J=6.8 Hz, 2H), 3.75-3.85 (m, 2H), 3.45-3.41 (m, 3H), 2.77 (s, 3H), 2.75-2.71 (m, 2H), 2.32-2.25 (m, 1H), 1.83-1.72 (m, 4H), 1.64-1.50 (m, 4H), 1.45-1.32 (m, 4H). LCMS (ESI) m/z: 504.2 [M+H]⁺.

### Example 2: 4-(cyclopentylmethoxy)-2-fluoro-5-methyl-N-((4-((1-methylazetidin-3-yl)oxy)piperidin-1-yl)sulfonyl)benzamide

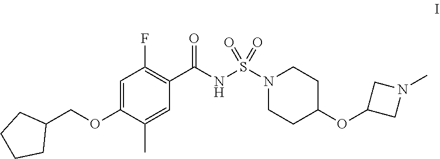 [SMILES: Cc1cc(C(=O)NS(=O)(=O)N2CCC(OC3CN(C)C3)CC2)c(F)cc1OCC1CCCC1]

### Step 1: tert-butyl 4-(cyclopentylmethoxy)-2-fluoro-5-methylbenzoate

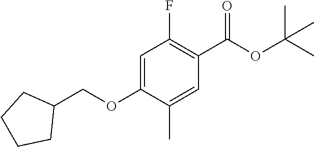 [SMILES: Cc1cc(C(=O)OC(C)(C)C)c(F)cc1OCC1CCCC1]

A mixture of tert-butyl 5-chloro-4-(cyclopentylmethoxy)-2-fluoro-benzoate (500 mg, 1.52 mmol), methylboronicacid (0.27 g, 4.56 mmol) and K₃PO₄ (0.97 g, 4.56 mmol) in toluene (7 mL) and water (1 mL) was added Pd(OAc)₂ (34 mg, 0.15 mmol) and dicyclohexyl-(2′,6′-dimethoxybiphenyl-2-yl)-phosphane (62 mg, 0.15 mmol). The mixture was stirred at 100° C. under nitrogen atmosphere for 3 h. After cooling to room temperature, the reaction was diluted with water (50 mL) and extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-10% EtOAc in petroleum ether) to afford the title compound (370 mg, 78%) as a yellow solid. LCMS (ESI) m/z: 253.1 [M-56+H]⁺.

### Step 2: 4-(cyclopentylmethoxy)-2-fluoro-5-methylbenzoic acid

To a solution of 2,2,2-trifluoroacetic acid (3.0 mL, 40.39 mmol) in DCM (6 mL) was added tert-butyl 4-(cyclopentylmethoxy)-2-fluoro-5-methyl-benzoate (370 mg, 1.2 mmol) at room temperature. The mixture was stirred at room temperature for 1 h. The mixture was concentrated in vacuo. The residue was added n-heptane (5 mL) and stirred at room temperature for 0.5 h. The resultant mixture was filtered to afford the title compound (130 mg, 42%) as a white solid that required no further purification. ¹H NMR (400 MHz, DMSO-d₆) δ 7.78 (d, J=8.4 Hz, 1H), 6.58 (d, J=12.4 Hz, 1H), 3.89 (d, J=6.8 Hz, 2H), 2.46-2.38 (m, 1H), 2.20 (s, 3H), 1.91-1.84 (m, 2H), 1.70-1.61 (m, 4H), 1.44-1.37 (m, 2H).

### Step 3: 4-(cyclopentylmethoxy)-2-fluoro-5-methyl-N-((4-((1-methylazetidin-3-yl)oxy)piperidin-1-yl)sulfonyl)benzamide

Following the procedure described in Example 1 and making non-critical variations as required to replace 5-chloro-4-(cyclopentylmethoxy)-2-fluoro-benzoic acid with 4-(cyclopentylmethoxy)-2-fluoro-5-methylbenzoic acid, the title compound was obtained as a white solid. ¹H NMR (400 MHz, DMSO-d₆) δ 7.46 (d, J=8.8 Hz, 1H), 6.73 (d, J=12.4 Hz, 1H), 4.40-4.30 (m, 1H), 4.15-4.04 (m, 2H), 3.87 (d, J=6.8 Hz, 2H), 3.63-3.61 (m, 2H), 3.47-3.35 (m, 3H), 2.85-2.75 (m, 2H), 2.67 (s, 3H), 2.35-2.24 (m, 1H), 2.09 (s, 3H), 1.86-1.71 (m, 4H), 1.68-1.48 (m, 4H), 1.47-1.29 (m, 4H). LCMS (ESI) m/z: 484.1 [M+H]⁺.

### Example 3: 4-(cyclopentylmethoxy)-5-ethyl-2-fluoro-N-((4-((1-methylazetidin-3-yl)oxy)piperidin-1-yl) sulfonyl) benzamide

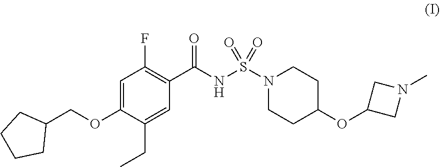 [SMILES: CCc1cc(C(=O)NS(=O)(=O)N2CCC(OC3CN(C)C3)CC2)c(F)cc1OCC1CCCC1]

### a. Step 1: 4-(cyclopentylmethoxy)-5-ethyl-2-fluorobenzoic acid

Following the procedure described in Example 2 and making non-critical variations as required to replace methylboronic acid with ethylboronic acid, the title compound was obtained as a white solid. LCMS (ESI) m/z: 266.9 [M+H]⁺.

### Step 2: 4-(cyclopentylmethoxy)-5-ethyl-2-fluoro-N-((4-((1-methylazetidin-3-yl) oxy) piperidin-1-yl) sulfonyl) benzamide

Following the procedure described in Example 1 and making non-critical variations as required to replace 5-chloro-4-(cyclopentylmethoxy)-2-fluorobenzoic acid with 4-(cyclopentylmethoxy)-5-ethyl-2-fluorobenzoic acid, the title compound was obtained as a white solid. ¹H NMR (400 MHz, DMSO-d₆) δ 7.43 (d, J=8.4 Hz, 1H), 6.80 (d, J=12.8 Hz, 1H), 4.42-4.34 (m, 1H), 4.30-4.13 (m, 2H), 3.90 (d, J=6.4 Hz, 2H), 3.78-3.69 (m, 2H), 3.53-3.42 (m, 3H), 2.98-2.89 (m, 2H), 2.73 (s, 3H), 2.52-2.47 (m, 2H), 2.46-2.42 (m, 1H), 1.88-1.73 (m, 4H), 1.66-1.53 (m, 4H), 1.50-1.42 (m, 2H), 1.40-1.30 (m, 2H), 1.12 (t, J=7.6 Hz, 3H). LCMS (ESI) m/z: 498.1 [M+H]⁺.

### Example 4: 4-(cyclopentylmethoxy)-5-cyclopropyl-2-fluoro-N-((4-((1-methylazetidin-3-yl)oxy)piperidin-1-yl) sulfonyl) benzamide

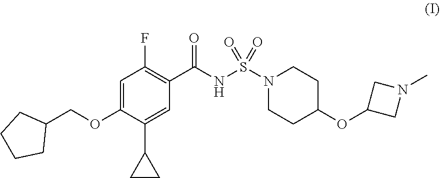 [SMILES: CN1CC(OC2CCN(S(=O)(=O)NC(=O)c3cc(C4CC4)c(OCC4CCCC4)cc3F)CC2)C1]

### Step 1: 4-(cyclopentylmethoxy)-5-cyclopropyl-2-fluorobenzoic acid

Following the procedure described in Example 2 and making non-critical variations as required to replace methylboronic acid with cyclopropylboronic acid, the title compound was obtained as a white solid. LCMS (ESI) m/z: 279.2 [M+H]⁺.

### Step 2: 4-(cyclopentylmethoxy)-5-cyclopropyl-2-fluoro-N-((4-((1-methylazetidin-3-yl) oxy) piperidin-1-yl) sulfonyl) benzamide

Following the procedure described in Example 1 and making non-critical variations as required to replace 5-chloro-4-(cyclopentylmethoxy)-2-fluorobenzoic acid with 4-(cyclopentyl-methoxy)-5-cyclopropyl-2-fluorobenzoic acid, the title compound was obtained as a white solid. ¹H NMR (400 MHz, DMSO-d₆) δ 7.15 (d, J=8.8 Hz, 1H), 6.74 (d, J=12.8 Hz, 1H), 4.38-4.27 (m, 1H), 4.10-4.03 (m, 2H), 3.90 (d, J=6.8 Hz, 2H), 3.62-3.50 (m, 2H), 3.45-3.35 (m, 3H), 2.90-2.78 (m, 2H), 2.65 (s, 3H), 2.37-2.28 (m, 1H), 2.03-1.95 (m, 1H), 1.85-1.73 (m, 4H), 1.64-1.51 (m, 4H), 1.46-1.29 (m, 4H), 0.90-0.84 (m, 2H), 0.61-0.54 (m, 2H). LCMS (ESI) m/z: 510.3 [M+H]⁺.

### Example 5: 4-(cyclopentylmethoxy)-2-fluoro-5-isopropyl-N-((4-((1-methylazetidin-3-yl)oxy)piperidin-1-yl)sulfonyl)benzamide

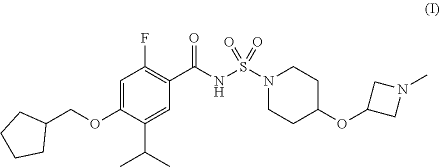 [SMILES: CC(C)c1cc(C(=O)NS(=O)(=O)N2CCC(OC3CN(C)C3)CC2)c(F)cc1OCC1CCCC1]

### Step 1: tert-butyl 4-(cyclopentylmethoxy)-2-fluoro-5-(prop-1-en-2-yl)benzoate

To a solution of tert-butyl 5-chloro-4-(cyclopentylmethoxy)-2-fluorobenzoate (200 mg, 0.61 mmol), Cs₂CO₃ (595 mg, 1.82 mmol) and 4,4,5,5-tetramethyl-2-(prop-1-en-2-yl)-1,3,2-dioxaborolane (123 mg, 0.73 mmol) in 1,4-dioxane (3 mL) and water (0.3 mL) was added XPhos Pd G2 (47 mg, 0.06 mmol) and XPhos (29 mg, 0.06 mmol) under nitrogen atmosphere. The reaction was stirred at 100° C. for 16 h. After cooling to room temperature, the reaction was diluted with water (20 mL) and extracted with EtOAc (20 mL×3). The combined organic layers were washed with brine, anhydrous Na₂SO₄, filtered and concentrated in vacuo. The residue was purified on silica gel chromatography (solvent gradient: 100% petroleum ether) to afford the title compound (150 mg, 74%) as light yellow oil. LCMS (ESI) m/z: 279.2 [M-56+H]⁺.

### Step 2: tert-butyl 4-(cyclopentylmethoxy)-2-fluoro-5-isopropyl-benzoate

To a solution of tert-butyl 4-(cyclopentylmethoxy)-2-fluoro-5-(prop-1-en-2-yl)benzoate (150 mg, 0.45 mmol) in EtOAc (5 mL) was added 10% Pd/C (71 mg, 0.07 mmol). The reaction was stirred under hydrogen atmosphere (15 psi) at room temperature for 16 h. The mixture was filtered and concentrated in vacuo to afford the title compound (150 mg, crude) as colorless oil that required no further purification. LCMS (ESI) m/z: 281.2 [M-56+H]⁺.

### Step 3: 4-(cyclopentylmethoxy)-2-fluoro-5-isopropylbenzoic acid

To a solution of tert-butyl 4-(cyclopentylmethoxy)-2-fluoro-5-isopropyl-benzoate (150 mg, 0.45 mmol) in DCM (2 mL) was added 2,2,2-trifluoroacetic acid (1 mL). The reaction was stirred at room temperature for 4 h. The mixture was concentrated in vacuo. The residue was added n-heptane (5 mL) and stirred at room temperature for 0.5 h. The resultant mixture was filtered to afford the title compound (120 mg, crude) as a light blue solid that required no further purification. LCMS (ESI) m/z: 281.1 [M+H]⁺.

### Step 4: 4-(cyclopentylmethoxy)-2-fluoro-5-isopropyl-N-((4-((1-methylazetidin-3-yl)oxy)piperidin-1-yl)sulfonyl)benzamide

Following the procedure described in Example 1 and making non-critical variations as required to replace 5-chloro-4-(cyclopentylmethoxy)-2-fluorobenzoic acid with 4-(cyclopentyl-methoxy)-2-fluoro-5-isopropylbenzoic acid, the title compound was obtained as a white solid. ¹H NMR (400 MHz, DMSO-d₆) δ 7.50 (d, J=8.8 Hz, 1H), 6.69 (d, J=12.8 Hz, 1H), 4.30-4.27 (m, 1H), 4.05-3.96 (m, 2H), 3.87 (d, J=6.4 Hz, 2H), 3.50-3.41 (m, 4H), 3.18-3.11 (m, 1H), 2.83-2.78 (m, 2H), 2.58 (s, 3H), 2.36-2.28 (m, 2H), 1.80-1.77 (m, 4H), 1.64-1.52 (m, 4H), 1.44-1.33 (m, 4H), 1.16 (d, J=7.2 Hz, 6H). LCMS (ESI) m/z: 512.3 [M+H]⁺.

### Example 6: 4-(cyclopentylmethoxy)-2-fluoro-5-methoxy-N-((4-((1-methylazetidin-3-yl)oxy)piperidin-1-yl) sulfonyl) benzamide

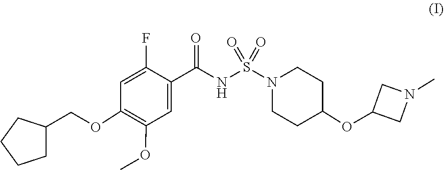 [SMILES: COc1cc(C(=O)NS(=O)(=O)N2CCC(OC3CN(C)C3)CC2)c(F)cc1OCC1CCCC1]

### Step 1: 1-bromo-4-(cyclopentylmethoxy)-2-fluoro-5-methoxybenzene

A solution of K₂CO₃ (1.13 g, 8.14 mmol), NaI (0.61 g, 4.07 mmol), (bromomethyl)cyclopentane (2.0 g, 12.2 mmol) and 4-bromo-5-fluoro-2-methoxyphenol (0.90 g, 4.07 mmol) in DMSO (10 mL) was stirred at 80° C. for 16 h. After cooling to room temperature, the reaction was diluted with water (50 mL) and extracted with EtOAc (50 mL×3). The combined organic layers were washed with brine (30 mL×4), dried over anhydrous Na₂SO₄, filtered and concentrated in vacuo. The crude residue was purified by silica gel chromatography (solvent gradient: 0-30% EtOAc in petroleum ether) to afford the title compound (0.60 g, 86%) as light yellow oil. ¹H NMR (400 MHz, CDCl₃) δ 6.97 (d, J=6.4 Hz, 1H), 6.70 (d, J=10.0 Hz, 1H), 3.85-3.82 (m, 5H), 2.49-2.46 (m, 1H), 1.90-1.82 (m, 2H), 1.68-1.58 (m, 4H), 1.38-1.32 (m, 2H).

### Step 2: 4-(cyclopentylmethoxy)-2-fluoro-5-methoxybenzoic acid

To a solution of 1-bromo-4-(cyclopentylmethoxy)-2-fluoro-5-methoxy-benzene (0.30 g, 0.99 mmol) in THF (6 mL) was added n-BuLi (0.47 mL, 1.19 mmol, 2.5 M) at −78° C. The mixture was stirred at −78° C. for 1 h. Then CO₂ gas (15 psi) was bubbled. The reaction was stirred at room temperature for 1 h. The reaction was quenched with water (2 mL), then adjusted to pH<7 with aqueous HCl (1 M), extracted with ethyl acetate (30 mL×3). The combined organic layers were washed with brine (50 mL), dried over anhydrous Na₂SO₄, filtered and concentrated in vacuo. The crude residue was purified by silica gel chromatography (solvent gradient: 0-50% EtOAc in petroleum ether) to afford the title compound (80 mg, 30%) as yellow oil. ¹H NMR (400 MHz, CDCl₃) δ 7.42 (d, J=4.0 Hz, 1H), 6.61 (d, J=12.4 Hz, 1H), 3.89 (d, J=7.2 Hz, 2H), 3.86 (s, 3H), 2.51-2.40 (m, 1H), 1.93-1.82 (m, 2H), 1.67-1.59 (m, 4H), 1.38-1.31 (m, 2H).

### Step 3: 4-(cyclopentylmethoxy)-2-fluoro-5-methoxy-N-((4-((1-methylazetidin-3-yl)oxy) piperidin-1-yl) sulfonyl) benzamide

Following the procedure described in Example 1 and making non-critical variations as required to replace 5-chloro-4-(cyclopentylmethoxy)-2-fluorobenzoic acid with 4-(cyclopentyl-methoxy)-2-fluoro-5-methoxybenzoic acid, the title compound was obtained as a white solid. ¹H NMR (400 MHz, DMSO-d₆) δ 7.23 (d, J=7.2 Hz, 1H), 6.78 (d, J=12.0 Hz, 1H), 4.39-4.28 (m, 1H), 4.11-4.03 (m, 2H), 3.85 (d, J=7.2 Hz, 2H), 3.74 (s, 3H), 3.62-3.57 (m, 1H), 3.44-3.42 (m, 2H), 2.81 (t, J=10.0 Hz, 2H), 2.66 (s, 3H), 2.34-2.26 (m, 1H), 1.85-1.70 (m, 4H), 1.64-1.50 (m, 4H), 1.49-1.40 (m, 2H), 1.36-1.27 (m, 2H). LCMS (ESI) m/z: 500.3 [M+H]⁺.

### Example 7: 5-cyclobutyl-4-(cyclopentylmethoxy)-2-fluoro-N-((4-((1-methylazetidin-3-yl)oxy)piperidin-1-yl)sulfonyl)benzamide 2,2,2-trifluoroacetate

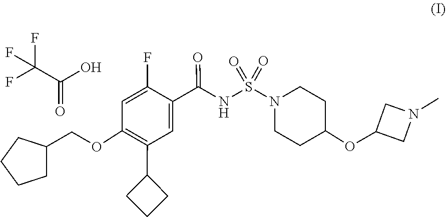

### Step 1: tert-butyl 5-bromo-4-(cyclopentylmethoxy)-2-fluorobenzoate

To a mixture of cyclopentanemethanol (1.4 g, 13.98 mmol) and tert-butyl 5-bromo-2,4-difluoro-benzoate (4.51 g, 15.38 mmol) in DMSO (12 mL) was added Cs₂CO₃ (4.55 g, 13.98 mmol) and the reaction mixture was heated at 80° C. for 16 h. After cooling to room temperature, the mixture was diluted with EtOAc (100 mL), washed with brine (100 mL×5). The organic lawyer was dried over Na₂SO₄, filtered, concentrated in vacuo. The residue was purified by reverse phase chromatography (acetonitrile 65-99%/(0.225% HCOOH) in water) to afford the title compound (1.75 g, 33%) as a white solid. ¹H NMR (400 MHz, CDCl₃) δ 8.05 (d, J=7.6 Hz, 1H), 6.60 (d, J=12.4 Hz, 1H), 3.92 (d, J=6.8 Hz, 2H), 2.37-2.49 (m, 1H), 1.92-1.83 (m, 2H), 1.74-1.60 (m, 4H), 1.58 (s, 9H), 1.45-1.37 (m, 2H). LCMS (ESI) m/z: 316.8 [M-56+H]⁺.

### Step 2: tert-butyl 5-cyclobutyl-4-(cyclopentylmethoxy)-2-fluorobenzoate

To a solution of tert-butyl 5-bromo-4-(cyclopentylmethoxy)-2-fluorobenzoate (300 mg, 0.8 mmol) in THF (4 mL) was added Pd(dppf)Cl₂ (117 mg, 0.16 mmol) and bromo(cyclobutyl)zinc (8.0 mL, 4.02 mmol) under nitrogen atmosphere. The reaction was stirred at 65° C. for 16 h. After cooling to 0° C., the reaction was quenched with water (10 mL) and extracted with EtOAc (20 mL×3). The combined organic layers were washed with brine (20 mL), dried over anhydrous Na₂SO₄, filtered and concentrated in vacuo. The crude residue was purified by silica gel chromatography (solvent gradient: 0-10% EtOAc in petroleum ether) to afford the title compound (140 mg, 50%) as yellow oil. LCMS (ESI) m/z: 293.1 [M-56+H]⁺.

### Step 3: 5-cyclobutyl-4-(cyclopentylmethoxy)-2-fluorobenzoic acid

To a solution of tert-butyl 5-cyclobutyl-4-(cyclopentylmethoxy)-2-fluorobenzoate (180 mg, 0.52 mmol) in DCM (5 mL) was added 2,2,2-trifluoroacetic acid (0.38 mL, 5.17 mmol). The reaction was stirred at room temperature for 1 h. The mixture was concentrated in vacuo, treated with n-heptane (5 mL) and stirred at room temperature for 0.5 h. The resultant mixture was filtered and the filtrate dried in vacuo to afford the title compound (140 mg, crude) as yellow oil that required no further purification. LCMS (ESI) m/z: 293.2 [M+H]⁺.

### Step 4: 5-cyclobutyl-4-(cyclopentylmethoxy)-2-fluoro-N-((4-((1-methylazetidin-3-yl)oxy)piperidin-1-yl)sulfonyl)benzamide 2,2,2-trifluoroacetate

Following the procedure described in Example 1 and making non-critical variations as required to replace 5-chloro-4-(cyclopentylmethoxy)-2-fluorobenzoic acid with 5-cyclobutyl-4-(cyclopentylmethoxy)-2-fluorobenzoic acid, the title compound was obtained as a white solid. ¹H NMR (400 MHz, DMSO-d₆) δ 11.70 (s, 1H), 7.36 (d, J=8.4 Hz, 1H), 6.91 (d, J=12.8 Hz, 1H), 4.50-4.35 (m, 2H), 4.12-4.00 (m, 2H), 3.90 (d, J=6.8 Hz, 2H), 3.89-3.80 (m, 1H), 3.63-3.50 (m, 4H), 3.45-3.41 (m, 2H), 3.39-3.35 (m, 2H), 3.15-3.05 (m, 2H), 2.83 (s, 3H), 2.37-2.29 (m, 1H), 2.28-2.20 (m, 2H), 2.11-1.96 (m, 3H), 1.90-1.83 (m, 2H), 1.81-1.76 (m, 2H), 1.64-1.54 (m, 6H), 1.38-1.28 (m, 2H), 1.25-1.22 (m, 1H). LCMS (ESI) m/z: 524.1 [M+H]⁺.

### Example 9: 5-cyclopropyl-4-(cyclopropylmethoxy)-2-fluoro-N-((4-((1-methylazetidin-3-yl)oxy)piperidin-1-yl)sulfonyl)benzamide

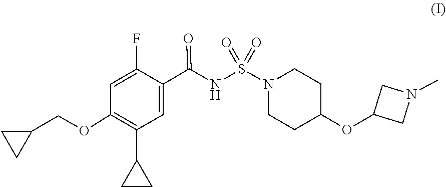 [SMILES: CN1CC(OC2CCN(S(=O)(=O)NC(=O)c3cc(C4CC4)c(OCC4CC4)cc3F)CC2)C1]

### Step 1: tert-butyl 4-(benzyloxy)-5-chloro-2-fluorobenzoate

To a solution of tert-butyl 5-chloro-2,4-difluoro-benzoate (5.0 g, 20.11 mmol) and Cs₂CO₃ (13.1 g, 40.22 mmol) in DMSO (50 mL), benzyl alcohol (2.17 g, 20.11 mmol) was added. The reaction was stirred at 80° C. under nitrogen atmosphere for 16 h. After cooling to room temperature, the reaction was diluted with water (100 mL) and extracted with EtOAc (100 mL×2). The combined organic layers were washed with brine (100 mL×3), dried over anhydrous Na₂SO₄, filtered and concentrated in vacuo. The crude residue was purified by silica gel chromatography (solvent gradient: 0-1% EtOAc in petroleum ether) to afford the title compound (4.7 g, 69%) as colorless oil. LCMS (ESI) m/z: 281.1 [M-56+H]⁺.

### Step 2: tert-butyl 4-(benzyloxy)-5-cyclopropyl-2-fluorobenzoate

To a solution of tert-butyl 4-benzyloxy-5-chloro-2-fluoro-benzoate (2.5 g, 7.42 mmol), K₃PO₄ (4.73 g, 22.27 mmol) and cyclopropylboronicacid (956 mg, 11.13 mmol) in toluene (17.5 mL) and water (2.5 mL), Pd(OAc)₂ (166 mg, 0.74 mmol) and 2-dicyclohexylphosphino-2′,6′-dimethoxybiphenyl (304 mg, 0.74 mmol) was added under nitrogen atmosphere at room temperature. The reaction was stirred at 100° C. for 16 h. After cooling to room temperature, the reaction was diluted with water (50 mL) and extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-2% EtOAc in petroleum ether) to afford the title compound (2.1 g, 82%) as colorless oil. LCMS (ESI) m/z: 287.1 [M-56+H]⁺.

### Step 3: tert-butyl 5-cyclopropyl-2-fluoro-4-hydroxybenzoate

To a solution of tert-butyl 4-(benzyloxy)-5-cyclopropyl-2-fluorobenzoate (1.4 g, 4.09 mmol) in ethanol (30 mL) and Pd/C (870 mg, 0.82 mmol) was added at room temperature. The mixture was stirred at room temperature under hydrogen atmosphere (15 psi) for 16 h. The reaction was filtered and concentrated in vacuo to afford the title compound (1.0 g, crude) as colorless oil that required no further purification. LCMS (ESI) m/z: 197.1 [M-56+H]⁺.

### Step 4: tert-butyl 5-cyclopropyl-4-(cyclopropylmethoxy)-2-fluorobenzoate

To a stirred solution of tert-butyl 5-cyclopropyl-2-fluoro-4-hydroxy-benzoate (0.25 g, 0.99 mmol) in DMF (2.5 mL) was added TBAI (0.04 g, 0.10 mmol), K₂CO₃ (0.55 g, 3.96 mmol) and cyclopropylmethyl bromide (0.67 g, 4.95 mmol) at room temperature under nitrogen atmosphere. Then reaction was stirred at 70° C. for 16 h. After cooling to room temperature, the reaction was diluted with water (50 mL) and extracted with EtOAc (50 mL×3). The combined organic layers were washed with brine (30 mL×3), dried over anhydrous Na₂SO₄, filtered and concentrated in vacuo to afford the title compound (300 mg, 92%) as yellow oil that required no further purification. LCMS (ESI) m/z: 251.2 [M-56+H]⁺.

### Step 5: 5-cyclopropyl-4-(cyclopropylmethoxy)-2-fluoro-N-((4-((1-methylazetidin-3-yl)oxy)piperidin-1-yl) sulfonyl)benzamide

Following the procedure described in Example 1 and making non-critical variations as required to replace 5-chloro-4-(cyclopentylmethoxy)-2-fluorobenzoic acid with 5-cyclopropyl-4-(cyclopropylmethoxy)-2-fluorobenzoic acid, the title compound was obtained as a white solid. ¹H NMR (400 MHz, DMSO-d₆) δ 7.14 (d, J=8.8 Hz, 1H), 6.67 (d, J=12.8 Hz, 1H), 4.35-4.23 (m, 1H), 4.05-3.94 (m, 2H), 3.87 (d, J=6.8 Hz, 2H), 3.55-3.45 (m, 5H), 2.85-2.70 (m, 2H), 2.59 (s, 3H), 2.10-1.95 (m, 1H), 1.85-1.70 (m, 2H), 1.50-1.35 (m, 2H), 1.30-1.15 (m, 1H), 0.93-0.80 (m, 2H), 0.63-0.52 (m, 4H), 0.39-0.31 (m, 2H). LCMS (ESI) m/z: 482.1 [M+H]⁺.

### Example 10: 5-chloro-4-(cyclopentylmethoxy)-2-fluoro-N-((4-((1-isopropylazetidin-3-yl)oxy)piperidin-1-yl)sulfonyl)benzamide

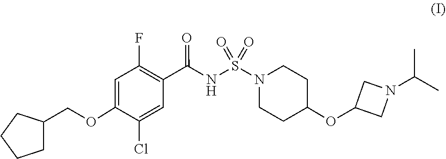 [SMILES: CC(C)N1CC(OC2CCN(S(=O)(=O)NC(=O)c3cc(Cl)c(OCC4CCCC4)cc3F)CC2)C1]

To a mixture of acetone (1.5 mL, 20.35 mmol) in acetonitrile (5 mL) was added N-[[4-(azetidin-3-yloxy)-1-piperidyl]sulfonyl]-5-chloro-4-(cyclopentylmethoxy)-2-fluoro-benzamide (50 mg, 0.10 mmol) and NaBH(OAc)₃ (32 mg, 0.15 mmol). The resulting mixture was stirred at room temperature for 16 h. The mixture was quenched with saturated aqueous NaHCO₃ (30 mL) to pH>7, extracted with EtOAc (30 mL×3). The combined organic layers were washed with brine (25 mL), dried over anhydrous Na₂SO₄, filtered and concentrated in vacuo. The crude residue was purified by reverse phase chromatography (acetonitrile 30-60%/(0.2% HCOOH) in water) to afford the title compound (11 mg, 21%) as a white solid. ¹H NMR (400 MHz, DMSO-d₆) δ 7.70 (d, J=7.6 Hz, 1H), 7.13 (d, J=11.2 Hz, 1H), 4.34-4.40 (m, 1H), 4.25-4.30 (m, 2H), 4.00 (d, J=6.8 Hz, 2H), 3.85-3.95 (m, 2H), 3.46-3.32 (m, 4H), 2.87-2.96 (m, 2H), 2.37-2.33 (m, 1H), 1.89-1.73 (m, 4H), 1.63-1.47 (m, 6H), 1.38-1.32 (m, 2H), 1.10 (d, J=6.4 Hz, 6H). LCMS (ESI) m/z: 532.1 [M+H]⁺.

### Example 11: 5-chloro-4-(cyclopentylmethoxy)-2-fluoro-N-((4-((1-(oxetan-3-yl)azetidin-3-yl)oxy)piperidin-1-yl)sulfonyl)benzamide

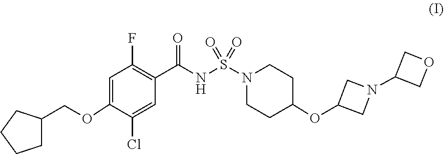 [SMILES: O=C(NS(=O)(=O)N1CCC(OC2CN(C3COC3)C2)CC1)c1cc(Cl)c(OCC2CCCC2)cc1F]

To a mixture of 3-oxetanone (29 mg, 0.41 mmol) in DCM (10 mL) was added N-[[4-(azetidin-3-yloxy)-1-piperidyl]sulfonyl]-5-chloro-4-(cyclopentylmethoxy)-2-fluoro-benzamide (100 mg, 0.20 mmol) and NaBH(OAc)₃ (86 mg, 0.41 mmol). The resulting mixture was stirred at room temperature for 16 h. The mixture was quenched with saturated aqueous NaHCO₃ (30 mL) to pH>7 and extracted with EtOAc (30 mL×3). The combined organic layers were washed with brine (25 mL), dried over anhydrous Na₂SO₄, filtered and concentrated in vacuo. The crude residue was purified by reverse phase chromatography (acetonitrile 30-60%/(0.05% NH₃·H₂O+10 mM NH₄HCO₃) in water) to afford the title compound (11 mg, 9%) as a white solid. ¹H NMR (400 MHz, DMSO-d₆) δ 7.71 (d, J=7.6 Hz, 1H), 6.96 (d, J=12.4 Hz, 1H), 4.76-4.72 (m, 2H), 4.50-4.45 (m, 2H), 4.37-4.30 (m, 1H), 3.99 (d, J=6.8 Hz, 2H), 3.91-3.85 (m, 1H), 3.78-3.73 (m, 2H), 3.64-3.59 (m, 2H), 3.57-3.52 (m, 1H), 3.24-3.18 (m, 4H), 2.46-2.39 (m, 1H), 1.92-1.85 (m, 4H), 1.72-1.61 (m, 6H), 1.49-1.42 (m, 2H). LCMS (ESI) m/z: 546.3 [M+H]⁺.

### Example 12: 5-chloro-4-(cyclopentylmethoxy)-N-((4-((1-cyclopropylazetidin-3-yl)oxy)piperidin-1-yl)sulfonyl)-2-fluorobenzamide

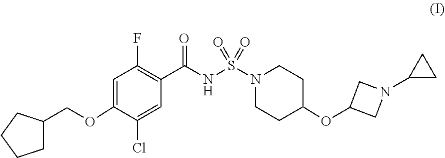 [SMILES: O=C(NS(=O)(=O)N1CCC(OC2CN(C3CC3)C2)CC1)c1cc(Cl)c(OCC2CCCC2)cc1F]

To a mixture of N-[[4-(azetidin-3-yloxy)-1-piperidyl]sulfonyl]-5-chloro-4-(cyclo-pentylmethoxy)-2-fluoro-benzamide (100 mg, 0.20 mmol) in EtOH (3 mL) was added (1-ethoxycyclopropoxy)trimethylsilane (213 mg, 1.22 mmol), acetic acid (0.01 ml, 0.20 mmol), NaBH₃CN (77 mg, 1.22 mmol), and 4 Å molecular sieve (100 mg). The resulting mixture was stirred at 50° C. for 1 h. After cooling to room temperature, the mixture was quenched with saturated aqueous NaHCO₃ (30 mL) (pH>7), diluted with water (10 mL) and extracted with EtOAc (20 mL×2). The combined organic layers were washed with brine (10 mL), dried over anhydrous Na₂SO₄, filtered and concentrated in vacuo. The crude residue was purified by reverse phase chromatography (acetonitrile 27-57%/(0.2% HCOOH) in water) to afford the title compound. ¹H NMR (400 MHz, DMSO-d₆) δ 7.71 (d, J=7.6 Hz, 1H), 7.11 (d, J=12.4 Hz, 1H), 4.25-4.15 (m, 1H), 3.99 (d, J=6.8 Hz, 2H), 3.75-3.83 (m, 2H), 3.50-3.40 (m, 5H), 3.00-2.85 (m, 2H), 2.40-2.20 (m, 2H), 1.91-1.69 (m, 4H), 1.67-1.50 (m, 4H), 1.48-1.26 (m, 4H), 0.58-0.27 (m, 4H). LCMS (ESI) m/z: 530.3 [M+H]⁺.

### Example 13: 5-chloro-4-(cyclopentylmethoxy)-2-fluoro-N-((4-((1-(2,2,2-trifluoroethyl)azetidin-3-yl)oxy)piperidin-1-yl)sulfonyl)benzamide

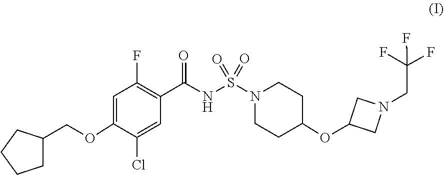 [SMILES: O=C(NS(=O)(=O)N1CCC(OC2CN(CC(F)(F)F)C2)CC1)c1cc(Cl)c(OCC2CCCC2)cc1F]

To a mixture of N-[[4-(azetidin-3-yloxy)-1-piperidyl]sulfonyl]-5-chloro-4-(cyclopentyl-methoxy)-2-fluoro-benzamide (80 mg, 0.16 mmol) in MeCN (2 mL) was added 2,2,2-trifluoroethyl trifluoromethanesulfonate (47 mg, 0.20 mmol) and NEt₃ (20 mg, 0.20 mmol). The resulting mixture was stirred at room temperature for 16 h. The reaction was diluted with water (10 mL) and extracted with EtOAc (10 mL×3). The combined organic layers were washed with brine (10 mL), dried over anhydrous Na₂SO₄, filtered and concentrated in vacuo. The crude residue was purified by reverse phase chromatography (acetonitrile 65-95%/(0.2% HCOOH) in water) to afford the title compound (5 mg, 5%). ¹H NMR (400 MHz, DMSO-d₆) δ 11.84 (s, 1H), 7.70 (d, J=7.6 Hz, 1H), 7.24 (d, J=12.0 Hz, 1H), 4.25-4.15 (m, 1H), 4.03 (d, J=6.8 Hz, 2H), 3.65-3.72 (m, 2H), 3.56-3.41 (m, 4H), 3.30-3.15 (m, 2H), 3.15-3.00 (m, 3H), 2.40-2.27 (m, 1H), 1.87-1.70 (m, 4H), 1.69-1.52 (m, 4H), 1.50-1.40 (m, 2H), 1.39-1.31 (m, 2H). LCMS (ESI) m/z: 572.2 [M+H]⁺.

### Example 14: 5-chloro-4-(cyclopentylmethoxy)-N-((3,4-dihydroquinolin-1(2H)-yl)sulfonyl)-2-fluorobenzamide

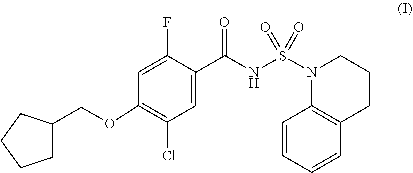 [SMILES: O=C(NS(=O)(=O)N1CCCc2ccccc21)c1cc(Cl)c(OCC2CCCC2)cc1F]

### Step 1: 3,4-dihydroquinoline-1(2H)-sulfonamide

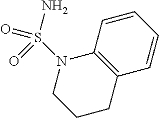 [SMILES: NS(=O)(=O)N1CCCc2ccccc21]

To a stirred solution of 1,2,3,4-tetrahydroquinoline (500 mg, 3.75 mmol) in 1-methyl-2-pyrrolidinone (22 mL) was added sulfamoylchloride (867 mg, 7.51 mmol) at 0° C. The mixture was stirred at room temperature under nitrogen atmosphere for 16 h. The reaction was diluted with water (50 mL) and extracted with EtOAc (50 mL×2). The combined organic layers were washed with brine (50 mL×4), dried over anhydrous Na₂SO₄, filtered and concentrated in vacuo to afford the title compound (550 mg, crude) as a yellow solid that required no further purification. LCMS (ESI) m/z: 213.2 [M+H]⁺.

### Step 2: 5-chloro-4-(cyclopentylmethoxy)-N-((3,4-dihydroquinolin-1(2H)-yl)sulfonyl)-2-fluorobenzamide

To a mixture of 3,4-dihydroquinoline-1(2H)-sulfonamide (195 mg, 0.92 mmol) and DMAP (224 mg, 1.83 mmol) in DCM (15 mL) was added 5-chloro-4-(cyclopentylmethoxy)-2-fluoro-benzoic acid (250 mg, 0.92 mmol) and EDCI (210 mg, 1.1 mmol). The resulting mixture was stirred at room temperature for 16 h under nitrogen atmosphere. The reaction was quenched with 10% citric aqueous solution (30 mL) and extracted with DCM (30 mL×3). The combined organic layers were washed with brine (20 mL), dried over anhydrous Na₂SO₄, filtered and concentrated in vacuo. The crude residue was purified by reverse phase chromatography (acetonitrile 70-100%/(0.2% HCOOH) in water) to afford the title compound (53 mg, 12%) as a white solid. ¹H NMR (400 MHz, DMSO-d₆) δ 12.45 (s, 1H), 7.60 (d, J=8.4 Hz, 1H), 7.53 (d, J=7.6 Hz, 1H), 7.24-7.10 (m, 3H), 7.05-6.97 (m, 1H), 4.00 (d, J=6.8 Hz, 2H), 3.90 (t, J=6.0 Hz, 2H), 2.73 (t, J=6.4 Hz, 2H), 2.38-2.25 (m, 1H), 2.00-1.90 (m, 2H), 1.82-1.70 (m, 2H), 1.68-1.47 (m, 4H), 1.40-1.27 (m, 2H). LCMS (ESI) m/z: 489.0 [M+Na]⁺.

### Example 15: 5-chloro-4-(cyclopentylmethoxy)-N-((3,4-dihydro-1,8-naphthyridin-1(2H)-yl)sulfonyl)-2-fluorobenzamide

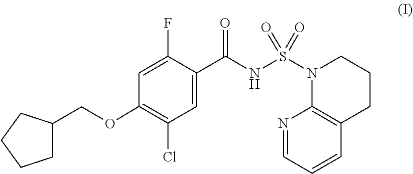 [SMILES: O=C(NS(=O)(=O)N1CCCc2cccnc21)c1cc(Cl)c(OCC2CCCC2)cc1F]

Following the procedure described in Example 14 and making non-critical variations as required to replace 1,2,3,4-tetrahydroquinoline with 1,2,3,4-tetrahydro-1,8-naphthyridine the title compound was obtained as a white solid. ¹H NMR (400 MHz, DMSO-d₆) δ 8.04-7.95 (m, 1H), 7.77 (d, J=7.6 Hz, 1H), 7.35-7.25 (m, 1H), 6.91 (d, J=13.2 Hz, 1H), 6.75-6.65 (m, 1H), 3.98-3.85 (m, 4H), 2.72-2.63 (m, 2H), 2.35-2.24 (m, 1H), 1.90-1.81 (m, 2H), 1.80-1.70 (m, 2H), 1.64-1.48 (m, 4H), 1.39-1.28 (m, 2H). LCMS (ESI) m/z: 468.1 [M+H]⁺.

### Example 16 and Example 17: (S)-5-chloro-4-(cyclopentylmethoxy)-2-fluoro-N-((3-methyl-3,4-dihydroquinolin-1(2H)-yl)sulfonyl)benzamide and (R)-5-chloro-4-(cyclopentylmethoxy)-2-fluoro-N-((3-methyl-3,4-dihydroquinolin-1(2H)-yl)sulfonyl)benzamide

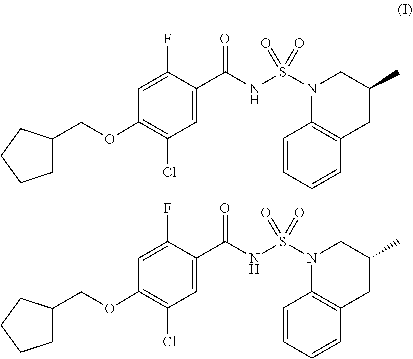

### Step 1: 5-chloro-4-(cyclopentylmethoxy)-2-fluoro-N-((3-methyl-3,4-dihydroquinolin-1(2H)-yl)sulfonyl)benzamide

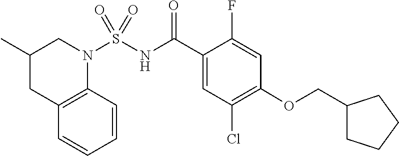 [SMILES: CC1Cc2ccccc2N(S(=O)(=O)NC(=O)c2cc(Cl)c(OCC3CCCC3)cc2F)C1]

Following the procedure described in Example 14 and making non-critical variations as required to replace 1,2,3,4-tetrahydroquinoline with 3-methyl-1,2,3,4-tetrahydroquinoline, the title compound was obtained as a white solid. LCMS (ESI) m/z: 481.2 [M+H]⁺.

### Step 2: (S)-4-(3-(dimethylamino)-3-phenethylpiperidin-1-yl)-2-fluoro-N-(pyrimidin-4-yl)benzenesulfonamide and (R)-4-(3-(dimethylamino)-3-phenethylpiperidin-1-yl)-2-fluoro-N-(pyrimidin-4-yl)benzenesulfonamide

5-chloro-4-(cyclopentylmethoxy)-2-fluoro-N-((3-methyl-3,4-dihydroquinolin-1(2H)-yl)sulfonyl)benzamide (140 mg, 0.29 mmol) was separated by using chiral SFC (Chiralpak IG (250 mm*30 mm, 10 um), Supercritical CO₂/EtOH+Heptane=95/5; 25 mL/min) to afford (S)-4-(3-(dimethylamino)-3-phenethylpiperidin-1-yl)-2-fluoro-N-(pyrimidin-4-yl)benzene-sulfonamide (26 mg, first peak) as a white solid and (R)-4-(3-(dimethylamino)-3-phenethyl-piperidin-1-yl)-2-fluoro-N-(pyrimidin-4-yl)benzenesulfonamide (24 mg, second peak) as a white solid. Absolute configuration was arbitrarily assigned to each enantiomer. Example 16: ¹H NMR (400 MHz, DMSO-d₆) δ 12.49 (s, 1H), 7.62-7.55 (m, 2H), 7.21-7.08 (m, 3H), 7.01-6.95 (m, 1H), 4.08-4.04 (m, 1H), 4.00 (d, J=6.8 Hz, 2H), 3.40-3.37 (m, 1H), 2.84-2.79 (m, 1H), 2.44-2.27 (m, 2H), 2.11-2.04 (m, 1H), 1.80-1.72 (m, 2H), 1.65-1.49 (m, 4H), 1.37-1.29 (m, 2H), 1.03 (d, J=6.4 Hz, 3H). LCMS (ESI) m/z: 481.2 [M+H]⁺. Example 17: ¹H NMR (400 MHz, DMSO-d₆) δ 12.50 (s, 1H), 7.60 (d, J=8.4 Hz, 1H), 7.56 (d, J=7.2 Hz, 1H), 7.20 (d, J=12 Hz, 1H), 7.17-7.11 (m, 2H), 7.02-6.98 (m, 1H), 4.08-4.04 (m, 1H), 4.00 (d, J=6.8 Hz, 2H), 3.40-3.37 (m, 1H), 2.85-2.79 (m, 1H), 2.44-2.37 (m, 1H), 2.36-2.28 (m, 1H), 2.12-2.04 (m, 1H), 1.80-1.72 (m, 2H), 1.63-1.50 (m, 4H), 1.37-1.29 (m, 2H), 1.03 (d, J=6.8 Hz, 3H). LCMS (ESI) m/z: 481.2 [M+H]⁺.

### Example 18: (R)-5-chloro-4-(cyclopentylmethoxy)-2-fluoro-N-((2-methyl-3,4-dihydro-quinolin-1(2H)-yl)sulfonyl)benzamide

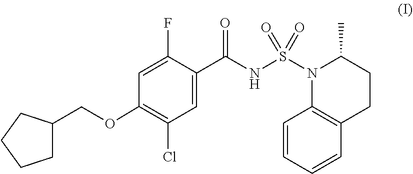 [SMILES: C[C@@H]1CCc2ccccc2N1S(=O)(=O)NC(=O)c1cc(Cl)c(OCC2CCCC2)cc1F]

Following the procedure described in Example 14 and making non-critical variations as required to replace 1,2,3,4-tetrahydroquinoline with 2-methyl-1,2,3,4-tetrahydroquinoline, 5-chloro-4-(cyclopentylmethoxy)-2-fluoro-N-[(2-methyl-3,4-dihydro-2H-quinolin-1-yl)sulfonyl]benzamide (420 mg, 0.87 mmol) was obtained as a white solid. The enantiomers were separated using chiral SFC (DAICEL CHIRALPAK AD (250 mm*30 mm, 10 um); Supercritical CO₂/EtOH+0.1% NH₃·H₂O=70/30; 70 mL/min) to afford the title compound (157 mg, first peak) as a white solid. Absolute configuration was arbitrarily assigned to each enantiomer. ¹H NMR (400 MHz, DMSO-d₆) δ 12.24 (s, 1H), 7.62 (d, J=8.0 Hz, 1H), 7.42 (d, J=7.6 Hz, 1H), 7.21-7.11 (m, 3H), 7.07-7.02 (m, 1H), 4.74-4.61 (m, 1H), 3.99 (d, J=6.4 Hz, 2H), 2.70-2.60 (m, 2H), 2.38-2.25 (m, 1H), 2.24-2.14 (m, 1H), 1.82-1.69 (m, 2H), 1.65-1.48 (m, 5H), 1.38-1.27 (m, 2H), 1.18 (d, J=6.4 Hz, 3H). LCMS (ESI) m/z: 481.2 [M+H]⁺.

### Example 19: 5-chloro-4-(cyclopentylmethoxy)-N-((3,4-dihydroisoquinolin-2(1H)-yl)sulfonyl)-2-fluorobenzamide

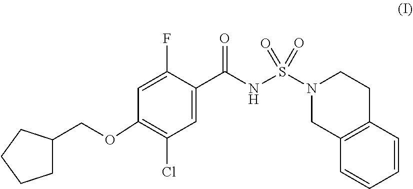 [SMILES: O=C(NS(=O)(=O)N1CCc2ccccc2C1)c1cc(Cl)c(OCC2CCCC2)cc1F]

### Step 1: 3,4-dihydroisoquinoline-2(1H)-sulfonamide

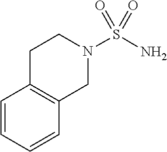 [SMILES: NS(=O)(=O)N1CCc2ccccc2C1]

To a mixture of 1,2,3,4-tetrahydroisoquinoline (500 mg, 3.75 mmol) in 1,4-dioxane (26 mL) was added sulfamide (902 mg, 9.39 mmol). The mixture was stirred at 110° C. for 16 h under nitrogen atmosphere. After cooling to room temperature, the reaction was diluted with water (30 mL) and extracted with EtOAc (30 mL×3). The combined organic layers were washed with brine (30 mL), dried over anhydrous Na₂SO₄, filtered and concentrated in vacuo to afford the title compound (670 mg, crude) as a yellow solid that required no further purification. LCMS (ESI) m/z: 212.8 [M+H]⁺.

### Step 2: 5-chloro-4-(cyclopentylmethoxy)-N-((3,4-dihydroisoquinolin-2(1H)-yl)sulfonyl)-2-fluorobenzamide

Following the procedure described in Example 14 and making non-critical variations as required to replace 3,4-dihydroquinoline-1(2H)-sulfonamide with 3,4-dihydroisoquinoline-2(1H)-sulfonamide, the title compound was obtained as a white solid. ¹H NMR (400 MHz, DMSO-d₆) δ 11.97 (s, 1H), 7.59 (d, J=7.2 Hz, 1H), 7.25-7.15 (m, 5H), 4.54 (s, 2H), 4.01 (d, J=6.8 Hz, 2H), 3.61 (t, J=5.6 Hz, 2H), 2.89 (t, J=5.6 Hz, 2H), 2.39-2.27 (m, 1H), 1.80-1.72 (m, 2H), 1.65-1.48 (m, 4H), 1.40-1.29 (m, 2H). LCMS (ESI) m/z: 467.0 [M+H]⁺.

### Example 20: 5-chloro-4-(cyclopentylmethoxy)-2-fluoro-N-((4-methoxypiperidin-1-yl)sulfonyl)benzamide

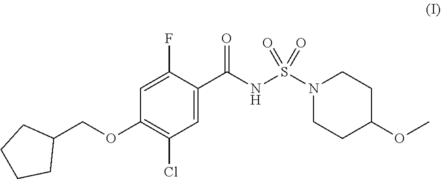 [SMILES: COC1CCN(S(=O)(=O)NC(=O)c2cc(Cl)c(OCC3CCCC3)cc2F)CC1]

Following the procedure described in Example 19 and making non-critical variations as required to replace 1,2,3,4-tetrahydroisoquinoline with 4-methoxypiperidine, the title compound was obtained as a white solid. ¹H NMR (400 MHz, DMSO-d₆) δ 11.82 (s, 1H), 7.71 (d, J=7.6 Hz, 1H), 7.24 (d, J=12.4 Hz, 1H), 4.03 (d, J=6.8 Hz, 2H), 3.53-3.37 (m, 3H), 3.23 (s, 3H), 3.19-3.12 (m, 2H), 2.38-2.30 (m, 1H), 1.88-1.73 (m, 4H), 1.64-1.49 (m, 6H), 1.40-1.32 (m, 2H). LCMS (ESI) m/z: 449.0 [M+H]⁺.

### Example 21: 5-chloro-4-(cyclopentylmethoxy)-2-fluoro-N-(piperidin-1-ylsulfonyl)benzamide

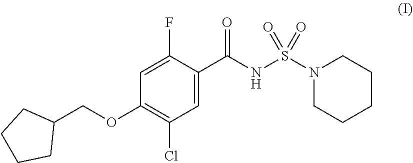 [SMILES: O=C(NS(=O)(=O)N1CCCCC1)c1cc(Cl)c(OCC2CCCC2)cc1F]

Following the procedure described in Example 19 and making non-critical variations as required to replace 1,2,3,4-tetrahydroisoquinoline with piperidine, the title compound was obtained as a white solid. ¹H NMR (400 MHz, DMSO-d₆) δ 11.77 (s, 1H), 7.71 (d, J=7.2 Hz, 1H), 7.23 (d, J=12.0 Hz, 1H), 4.03 (d, J=6.8 Hz, 2H), 3.30-3.21 (m, 4H), 2.40-2.28 (m, 1H), 1.84-1.71 (m, 2H), 1.68-1.59 (m, 2H), 1.59-1.52 (m, 6H), 1.51-1.43 (m, 2H), 1.41-1.29 (m, 2H). LCMS (ESI) m/z: 419.0 [M+H]⁺.

### Example 22: 5-chloro-4-(cyclopentylmethoxy)-2-fluoro-N-(pyrrolidin-1-ylsulfonyl)-benzamide

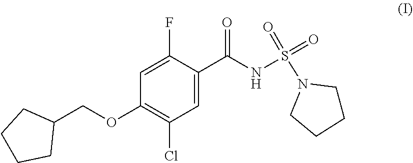 [SMILES: O=C(NS(=O)(=O)N1CCCC1)c1cc(Cl)c(OCC2CCCC2)cc1F]

Following the procedure described in Example 19 and making non-critical variations as required to replace 1,2,3,4-tetrahydroisoquinoline with pyrrolidine, the title compound was obtained as a white solid. ¹H NMR (400 MHz, DMSO-d₆) δ 11.75 (s, 1H), 7.71 (d, J=8.0 Hz, 1H), 7.21 (d, J=12.4 Hz, 1H), 4.02 (d, J=6.8 Hz, 2H), 3.43-3.37 (m, 4H), 2.36-2.31 (m, 1H), 1.85-1.81 (m, 4H), 1.80-1.73 (m, 2H), 1.67-1.59 (m, 2H), 1.58-1.51 (m, 2H), 1.40-1.30 (m, 2H). LCMS (ESI) m/z: 405.2 [M+H]⁺.

### Example 23: 5-chloro-4-(cyclopentylmethoxy)-2-fluoro-N-((octahydro-2H-pyrazino[1,2-a]pyrazin-2-yl)sulfonyl)benzamide

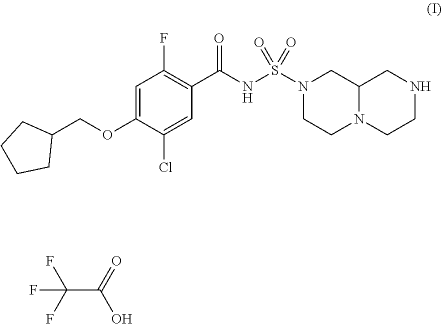 [SMILES: O=C(NS(=O)(=O)N1CCN2CCNCC2C1)c1cc(Cl)c(OCC2CCCC2)cc1F ; O=C(O)C(F)(F)F]

(This compound may also be referred to as: 5-chloro-4-(cyclopentylmethoxy)-2-fluoro-N-((hexahydro-1H-pyrazino[1,2-a]pyrazin-2(6H)-yl)sulfonyl)benzamide 2,2,2-trifluoroacetate.)

### Step 1: tert-butyl 8-(N-(5-chloro-4-(cyclopentylmethoxy)-2-fluorobenzoyl)sulfamoyl)hexahydro-1H-pyrazino[1,2-a]pyrazine-2(6H)-carboxylate

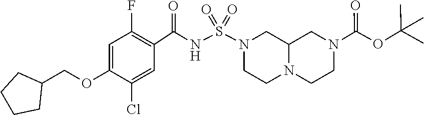 [SMILES: CC(C)(C)OC(=O)N1CCN2CCN(S(=O)(=O)NC(=O)c3cc(Cl)c(OCC4CCCC4)cc3F)CC2C1]

Following the procedure described in Example 19 and making non-critical variations as required to replace 1,2,3,4-tetrahydroisoquinoline with tert-butyl hexahydro-1H-pyrazino[1,2-a]pyrazine-2(6H)-carboxylate, the title compound was obtained as yellow oil. LCMS (ESI) m/z: 575.1 [M+H]⁺.

### Step 2: 5-chloro-4-(cyclopentylmethoxy)-2-fluoro-N-((hexahydro-1H-pyrazino[1,2-a]pyrazin-2(6H)-yl)sulfonyl)benzamide 2,2,2-trifluoroacetate

To a stirred solution of tert-butyl 8-(N-(5-chloro-4-(cyclopentylmethoxy)-2-fluorobenzoyl)sulfamoyl)hexahydro-1H-pyrazino[1,2-a]pyrazine-2(6H)-carboxylate (310 mg, 0.54 mmol) in DCM (4 mL) was added 2,2,2-trifluoroacetic acid (2 mL, 23.24 mmol). The mixture was stirred at room temperature for 1 h. The mixture was concentrated in vacuo. The crude residue was purified by reverse phase chromatography (acetonitrile 30-60%/(0.075% 2,2,2-trifluoroacetic acid) in water) to afford the title compound (103 mg, 32%) as a white solid. ¹H NMR (400 MHz, DMSO-d₆) δ 11.98 (s, 1H), 8.97-8.60 (m, 2H), 7.73 (d, J=7.6 Hz, 1H), 7.24 (d, J=12.4 Hz, 1H), 4.03 (d, J=6.8 Hz, 2H), 3.71-3.60 (m, 2H), 3.36-3.24 (m, 2H), 3.10-2.93 (m, 1H), 2.92-2.83 (m, 2H), 2.80-2.60 (m, 2H), 2.41-2.30 (m, 2H), 2.27-2.18 (m, 1H), 1.85-1.70 (m, 2H), 1.68-1.49 (m, 4H), 1.42-1.28 (m, 2H). LCMS (ESI) m/z: 475.1 [M+H]⁺.

### Example 24: (R)—N-((2-(aminomethyl)morpholino)sulfonyl)-5-chloro-4-(cyclopentyl-methoxy)-2-fluorobenzamide 2,2,2-trifluoroacetate

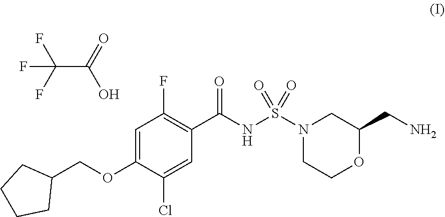 [SMILES: NC[C@@H]1CN(S(=O)(=O)NC(=O)c2cc(Cl)c(OCC3CCCC3)cc2F)CCO1 ; O=C(O)C(F)(F)F]

### a. Step 1: tert-butyl ((4-(N-(5-chloro-4-(cyclopentylmethoxy)-2-fluorobenzoyl)-sulfamoyl)morpholin-2-yl)methyl)carbamate

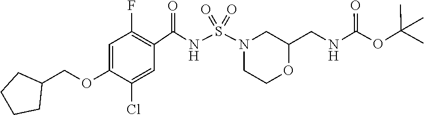 [SMILES: CC(C)(C)OC(=O)NCC1CN(S(=O)(=O)NC(=O)c2cc(Cl)c(OCC3CCCC3)cc2F)CCO1]

Following the procedure described in Example 19 and making non-critical variations as required to replace 1,2,3,4-tetrahydroisoquinoline with tert-butyl (morpholin-2-ylmethyl)carbamate, the title compound was obtained as a white solid. LCMS (ESI) m/z: 450.2 [M-100+H]⁺.

### Step 2: (R)-tert-butyl ((4-(N-(5-chloro-4-(cyclopentylmethoxy)-2-fluorobenzoyl)sulfamoyl)morpholin-2-yl)methyl)carbamate and (S)-tert-butyl ((4-(N-(5-chloro-4-(cyclopentylmethoxy)-2-fluorobenzoyl)sulfamoyl)-morpholin-2-yl)methyl)carbamate

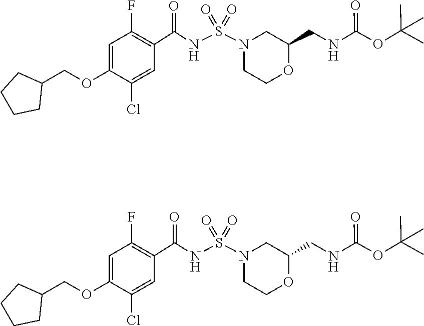 [SMILES: CC(C)(C)OC(=O)NC[C@@H]1CN(S(=O)(=O)NC(=O)c2cc(Cl)c(OCC3CCCC3)cc2F)CCO1 ; CC(C)(C)OC(=O)NC[C@H]1CN(S(=O)(=O)NC(=O)c2cc(Cl)c(OCC3CCCC3)cc2F)CCO1]

tert-butyl ((4-(N-(5-chloro-4-(cyclopentylmethoxy)-2-fluorobenzoyl)sulfamoyl) morpholin-2-yl)methyl)carbamate (100 mg, 0.18 mmol) was separated by using chiral SFC (Chiralpak AD (250 mm*30 mm, 10 um), Supercritical CO₂/EtOH+0.1% NH₃·H₂O=65/35; 70 mL/min) to afford (R)-tert-butyl ((4-(N-(5-chloro-4-(cyclopentylmethoxy)-2-fluorobenzoyl)sulfamoyl)morpholin-2-yl)methyl)carbamate (40 mg, first peak) and (S)-tert-butyl ((4-(N-(5-chloro-4-(cyclopentylmethoxy)-2-fluorobenzoyl)sulfamoyl) morpholin-2-yl)methyl)carbamate (30 mg, second peak). Absolute configuration was arbitrarily assigned to each enantiomer. LCMS (ESI) m/z: 450.2 [M-100+H]⁺.

### Step 3: (R)—N-((2-(aminomethyl)morpholino)sulfonyl)-5-chloro-4-(cyclopentylmethoxy)-2-fluorobenzamide 2,2,2-trifluoroacetate

Following the procedure described in Example 23 and making non-critical variations as required to replace tert-butyl 8-(N-(5-chloro-4-(cyclopentylmethoxy)-2-fluorobenzoyl)sulfamoyl)hexahydro-1H-pyrazino[1,2-a]pyrazine-2(6H)-carboxylate with (R)-tert-butyl ((4-(N-(5-chloro-4-(cyclopentylmethoxy)-2-fluorobenzoyl)sulfamoyl)morpholin-2-yl)methyl)carbamate, the title compound was obtained as a white solid. ¹H NMR (400 MHz, DMSO-d₆) δ 12.02 (s, 1H), 7.95-7.84 (s, 3H), 7.75 (d, J=7.2 Hz, 1H), 7.24 (d, J=12.4 Hz, 1H), 4.03 (d, J=6.8 Hz, 2H), 4.10-3.96 (m, 1H), 3.72-3.65 (m, 2H), 3.58-3.48 (m, 2H), 3.15-2.99 (m, 2H), 2.93-2.76 (m, 2H), 2.40-2.28 (m, 1H), 1.85-1.70 (m, 2H), 1.69-1.45 (m, 4H), 1.42-1.29 (m, 2H). LCMS (ESI) m/z: 450.2 [M+H]⁺.

### Example 25: (R)—N-((2-(aminomethyl)morpholino)sulfonyl)-4-(cyclopentylmethoxy)-5-cyclopropyl-2-fluorobenzamide 2,2,2-trifluoroacetate

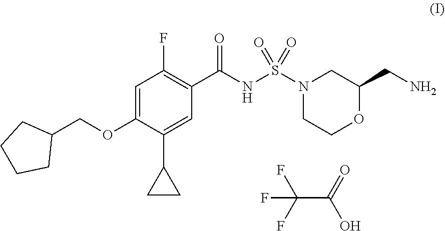

### a. Step 1: (R)-tert-butyl ((4-(N-(4-(cyclopentylmethoxy)-5-cyclopropyl-2-fluorobenzoyl)sulfamoyl)morpholin-2-yl)methyl)carbamate and (S)-tert-butyl ((4-(N-(4-(cyclopentylmethoxy)-5-cyclopropyl-2-fluorobenzoyl)sulfamoyl)morpholin-2-yl)methyl)carbamate

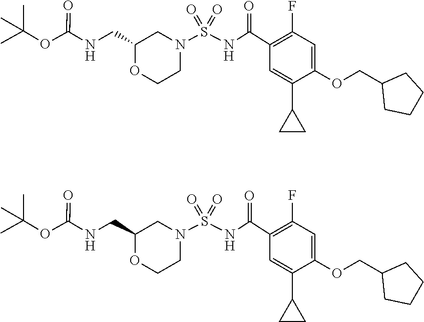 [SMILES: CC(C)(C)OC(=O)NC[C@@H]1CN(S(=O)(=O)NC(=O)c2cc(C3CC3)c(OCC3CCCC3)cc2F)CCO1 ; CC(C)(C)OC(=O)NC[C@H]1CN(S(=O)(=O)NC(=O)c2cc(C3CC3)c(OCC3CCCC3)cc2F)CCO1]

Following the procedure described in Example 19 and making non-critical variations as required to replace 1,2,3,4-tetrahydroisoquinoline with tert-butyl (morpholin-2-ylmethyl)carbamate and replace 5-chloro-4-(cyclopentylmethoxy)-2-fluorobenzoic acid with 4-(cyclopentylmethoxy)-5-cyclopropyl-2-fluorobenzoic acid, the title compound was obtained as yellow oil. Tert-butyl ((4-(N-(4-(cyclopentylmethoxy)-5-cyclopropyl-2-fluorobenzoyl)sulfamoyl)morpholin-2-yl)methyl)carbamate (230 mg, 0.41 mmol) was separated by using chiral SFC (Chiralpak AD (250 mm*30 mm, 10 μm), Supercritical CO₂/MeOH+0.1% NH₃·H₂O=55/45; 60 mL/min) to afford (R)-tert-butyl ((4-(N-(4-(cyclopentylmethoxy)-5-cyclopropyl-2-fluorobenzoyl)sulfamoyl)morpholin-2-yl)methyl)carbamate (110 mg, first peak) and (S)-tert-butyl ((4-(N-(4-(cyclopentylmethoxy)-5-cyclopropyl-2-fluorobenzoyl)-sulfamoyl)morpholin-2-yl)methyl)carbamate (90 mg, second peak) both as yellow oil. Absolute configuration was arbitrarily assigned to each enantiomer. LCMS (ESI) m/z: 578.1 [M+Na]⁺.

### Step 2: (R)—N-((2-(aminomethyl)morpholino)sulfonyl)-4-(cyclopentylmethoxy)-5-cyclopropyl-2-fluorobenzamide 2,2,2-trifluoroacetate

Following the procedure described in Example 23 and making non-critical variations as required to replace 8-(N-(5-chloro-4-(cyclopentylmethoxy)-2-fluorobenzoyl)sulfamoyl)hexahydro-1H-pyrazino[1,2-a]pyrazine-2(6H)-carboxylate with (R)-tert-butyl ((4-(N-(4-(cyclopentylmethoxy)-5-cyclopropyl-2-fluorobenzoyl)sulfamoyl)morpholin-2-yl)methyl)carbamate, the title compound was obtained as a white solid. ¹H NMR (400 MHz, DMSO-d₆) δ 11.82 (s, 1H), 7.96-7.80 (m, 3H), 7.11 (d, J=8.4 Hz, 1H), 6.96 (d, J=12.8 Hz, 1H), 4.05-3.97 (m, 1H), 3.96 (d, J=6.8 Hz, 2H), 3.74-3.62 (m, 2H), 3.58-3.48 (m, 2H), 3.16-2.97 (m, 2H), 2.94-2.76 (m, 2H), 2.40-2.28 (m, 1H), 2.07-1.94 (m, 1H), 1.85-1.72 (m, 2H), 1.68-1.48 (m, 4H), 1.45-1.29 (m, 2H), 0.94-0.84 (m, 2H), 0.72-0.62 (m, 2H). LCMS (ESI) m/z: 456.2 [M+H]⁺.

### Example 26: (R)-5-chloro-4-(cyclopentylmethoxy)-2-fluoro-N-((2-((methylamino) methyl)morpholino)sulfonyl) benzamide 2,2,2-trifluoroacetate

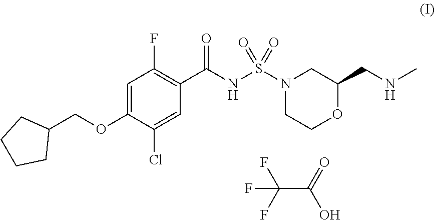

### Step 1: Benzyl 2-(((tert-butoxycarbonyl)amino)methyl)morpholine-4-carboxylate

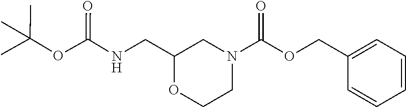 [SMILES: CC(C)(C)OC(=O)NCC1CN(C(=O)OCc2ccccc2)CCO1]

To a solution of tert-butyl N-(morpholin-2-ylmethyl)carbamate (500 mg, 2.31 mmol) and benzyl chloroformate (0.39 mL, 2.77 mmol) in DCM (16 mL) was added DIPEA (1.0 mL, 5.78 mmol) dropwise at 0° C. After stirring for 0.5 h, the reaction was stirred at room temperature for 16 h. The reaction was diluted with water (30 mL) and extracted with DCM (30 mL×2). The combined organic layers were washed with brine (30 mL), dried over anhydrous Na₂SO₄, filtered and concentrated in vacuo. The crude residue was purified by silica gel chromatography (solvent gradient: 0-60% EtOAc in petroleum ether) to afford the title compound (715 mg, 88%) as yellow oil. LCMS (ESI) m/z: 251.2 [M-100+H]⁺.

### Step 2: benzyl 2-(((tert-butoxycarbonyl)(methyl)amino)methyl)morpholine-4-carboxylate

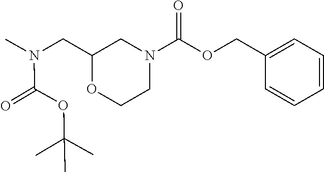 [SMILES: CN(CC1CN(C(=O)OCc2ccccc2)CCO1)C(=O)OC(C)(C)C]

To a solution of benzyl 2-(((tert-butoxycarbonyl) amino) methyl) morpholine-4-carboxylate (405 mg, 1.16 mmol) in THF (11 mL) was added NaH (92 mg, 2.31 mmol) at room temperature under nitrogen atmosphere. After the mixture was stirred for 30 min, Mel (600 mg, 3.32 mmol) was added slowly at room temperature under nitrogen atmosphere. The mixture was stirred at room temperature for 16 h. The reaction was quenched with saturated aqueous NH₄Cl solution (20 mL), extracted with EtOAc (50 mL×2). The combined organic layers were washed with brine (30 mL), dried over anhydrous Na₂SO₄, filtered and concentrated in vacuo to afford the title compound (415 mg, crude) as yellow oil that required no further purification. LCMS (ESI) m/z: 265.2 [M-100+H]⁺.

### Step 3: tert-butyl methyl(morpholin-2-ylmethyl)carbamate

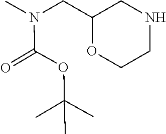 [SMILES: CN(CC1CNCCO1)C(=O)OC(C)(C)C]

To a solution of benzyl 2-[[tert-butoxycarbonyl(methyl)amino]methyl]morpholine-4-carboxylate (415 mg, 1.14 mmol) in EtOAc (16 mL) was added 10% Pd/C (606 mg, 0.57 mmol). The mixture was stirred at room temperature for 1 h under hydrogen atmosphere (15 psi). The mixture was filtered and the filtrate was concentrated in vacuo to afford the title compound (235 mg, crude) as yellow oil that required no further purification.

### Step 4: (R)-tert-butyl ((4-(N-(5-chloro-4-(cyclopentylmethoxy)-2-fluorobenzoyl) sulfamoyl) morpholin-2-yl)methyl)(methyl)carbamate and (S)-tert-butyl ((4-(N-(5-chloro-4-(cyclopentylmethoxy)-2-fluorobenzoyl) sulfamoyl) morpholin-2-yl)methyl)(methyl)carbamate

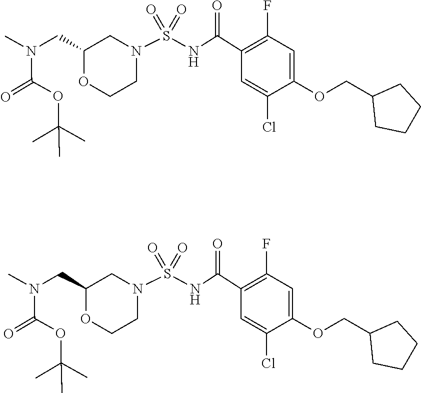 [SMILES: CN(C[C@@H]1CN(S(=O)(=O)NC(=O)c2cc(Cl)c(OCC3CCCC3)cc2F)CCO1)C(=O)OC(C)(C)C ; CN(C[C@H]1CN(S(=O)(=O)NC(=O)c2cc(Cl)c(OCC3CCCC3)cc2F)CCO1)C(=O)OC(C)(C)C]

Following the procedure described in Example 24 and making non-critical variations as required to replace tert-butyl (morpholin-2-ylmethyl)carbamate with tert-butyl methyl (morpholin-2-ylmethyl)carbamate, tert-butyl ((4-(N-(5-chloro-4-(cyclopentylmethoxy)-2-fluorobenzoyl)sulfamoyl)morpholin-2-yl)methyl)(methyl)carbamate was obtained as a white solid. Tert-butyl ((4-(N-(5-chloro-4-(cyclopentylmethoxy)-2-fluorobenzoyl)sulfamoyl)morpholin-2-yl)methyl)(methyl)carbamate (190 mg, 0.34 mmol) was separated by using chiral SFC (Chiralpak AD (250 mm*30 mm, 10 um), Supercritical CO₂/EtOH+0.1% NH₃·H₂O=55/45; 80 mL/min) to afford (R)-tert-butyl ((4-(N-(5-chloro-4-(cyclopentylmethoxy)-2-fluorobenzoyl)sulfamoyl)morpholin-2-yl)methyl)(methyl)carbamate (60 mg, first peak) and (S)-tert-butyl ((4-(N-(5-chloro-4-(cyclopentylmethoxy)-2-fluorobenzoyl)sulfamoyl)morpholin-2-yl)methyl)(methyl)carbamate (60 mg, second peak) both as a white solid. Absolute configuration was arbitrarily assigned to each enantiomer. LCMS (ESI) m/z. 464.2 [M-100+H]⁺.

### Step 5: (R)-5-chloro-4-(cyclopentylmethoxy)-2-fluoro-N-((2-((methylamino)methyl)morpholino)sulfonyl)benzamide 2,2,2-trifluoroacetate

To a stirred solution of (R)-tert-butyl ((4-(N-(5-chloro-4-(cyclopentylmethoxy)-2-fluorobenzoyl)sulfamoyl)morpholin-2-yl)methyl)(methyl)carbamate (60 mg, 0.11 mmol) in DCM (1 mL) was added 2,2,2-trifluoroacetic acid (0.5 mL) at room temperature for 1 h. The mixture was concentrated in vacuo. The crude residue was purified by reverse phase chromatography (acetonitrile 25-55%/0.2% HCOOH in water) to afford the title compound (15 mg, 25%) as a white solid. ¹H NMR (400 MHz, DMSO-d₆) δ 8.47 (s, 2H), 7.74 (d, J=8.0 Hz, 1H), 6.97 (d, J=12.4 Hz, 1H), 3.98-3.89 (m, 3H), 3.76-3.68 (m, 1H), 3.58-3.50 (m, 1H), 3.43-3.36 (m, 2H), 3.28-3.25 (m, 1H), 3.14-3.08 (m, 1H), 3.05-2.96 (m, 1H), 2.83-2.74 (m, 1H), 2.55 (s, 3H), 2.34-2.23 (m, 1H), 1.82-1.72 (m, 2H), 1.67-1.48 (m, 4H), 1.40-1.29 (m, 2H). LCMS (ESI) m/z: 464.2 [M+H]⁺.

### Example 27: (R)—N-((3-(aminomethyl)piperidin-1-yl)sulfonyl)-5-chloro-4-(cyclopentyl-methoxy)-2-fluorobenzamide 2,2,2-trifluoroacetate

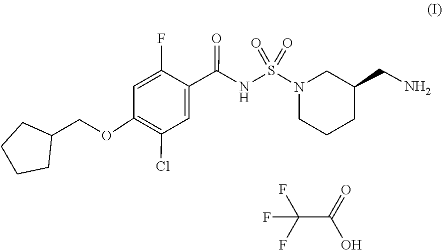 [SMILES: NC[C@H]1CCCN(S(=O)(=O)NC(=O)c2cc(Cl)c(OCC3CCCC3)cc2F)C1 ; O=C(O)C(F)(F)F]

### Step 1: (R)-tert-butyl ((1-(N-(5-chloro-4-(cyclopentylmethoxy)-2-fluorobenzoyl)-sulfamoyl)piperidin-3-yl)methyl)carbamate, and (S)-tert-butyl ((1-(N-(5-chloro-4-(cyclopentylmethoxy)-2-fluorobenzoyl)sulfamoyl)-piperidin-3-yl)methyl)carbamate

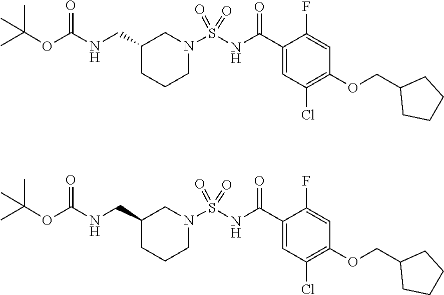 [SMILES: CC(C)(C)OC(=O)NC[C@H]1CCCN(S(=O)(=O)NC(=O)c2cc(Cl)c(OCC3CCCC3)cc2F)C1 ; CC(C)(C)OC(=O)NC[C@@H]1CCCN(S(=O)(=O)NC(=O)c2cc(Cl)c(OCC3CCCC3)cc2F)C1]

Following the procedure described in Example 24 and making non-critical variations as required to replace tert-butyl (morpholin-2-ylmethyl)carbamate with tert-butyl (piperidin-3-ylmethyl)carbamate, tert-butyl ((1-(N-(5-chloro-4-(cyclopentylmethoxy)-2-fluorobenzoyl)sulfamoyl)piperidin-3-yl)methyl)carbamate was obtained as a white solid. tert-butyl ((1-(N-(5-chloro-4-(cyclopentylmethoxy)-2-fluorobenzoyl)sulfamoyl)piperidin-3-yl)methyl)carbamate (180 mg, 0.33 mmol) was separated by using chiral SFC (DAICEL CHIRALCEL OD-H (250 mm*30 mm, 5 um); Supercritical CO₂/EtOH+0.1% NH₃·H₂O=50/50; 70 ml/min) to afford (R)-tert-butyl ((1-(N-(5-chloro-4-(cyclopentylmethoxy)-2-fluorobenzoyl)sulfamoyl)piperidin-3-yl)methyl)carbamate (60 mg, first peak) and (S)-tert-butyl ((1-(N-(5-chloro-4-(cyclopentylmethoxy)-2-fluorobenzoyl)sulfamoyl)piperidin-3-yl)methyl)carbamate (60 mg, second peak) both as a white solid. Absolute configuration was arbitrarily assigned to each enantiomer. LCMS (ESI) m/z: 448.0 [M-100+H]⁺.

### Step 2: (R)—N-((3-(aminomethyl)piperidin-1-yl)sulfonyl)-5-chloro-4-(cyclopentylmethoxy)-2-fluorobenzamide 2,2,2-trifluoroacetate

Following the procedure described in Example 24 and making non-critical variations as required to replace (R)-tert-butyl ((4-(N-(5-chloro-4-(cyclopentylmethoxy)-2-fluorobenzoyl)sulfamoyl)morpholin-2-yl)methyl)carbamate with (R)-tert-butyl ((1-(N-(5-chloro-4-(cyclopentylmethoxy)-2-fluorobenzoyl)sulfamoyl)piperidin-3-yl)methyl)carbamate, the title compound was obtained as a white solid. ¹H NMR (400 MHz, DMSO-d₆) δ 11.89 (s, 1H), 7.83 (s, 3H), 7.71 (d, J=7.6 Hz, 1H), 7.23 (d, J=12.8 Hz, 1H), 4.02 (d, J=7.2 Hz, 2H), 3.77 (d, J=10.0 Hz, 1H), 3.55 (d, J=12.0 Hz, 1H), 2.92-2.70 (m, 4H), 2.36-2.27 (m, 1H), 1.84-1.72 (m, 5H), 1.66-1.30 (m, 7H), 1.19-1.08 (m, 1H). LCMS (ESI) m/z: 448.0 [M+H]⁺.

### Example 28: (R)-5-chloro-4-(cyclopentylmethoxy)-N-((2-((dimethylamino)methyl)-morpholino)sulfonyl)-2-fluorobenzamide

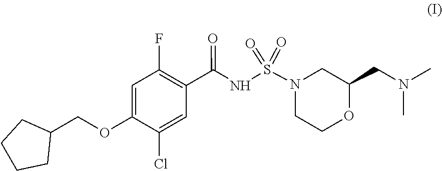 [SMILES: CN(C)C[C@@H]1CN(S(=O)(=O)NC(=O)c2cc(Cl)c(OCC3CCCC3)cc2F)CCO1]

### Step 1: (R)-tert-butyl ((4-(N-(5-chloro-4-(cyclopentylmethoxy)-2-fluorobenzoyl)sulfamoyl) morpholin-2-yl)methyl)carbamate and (S)-tert-butyl ((4-(N-(5-chloro-4-(cyclopentylmethoxy)-2-fluorobenzoyl)sulfamoyl)morpholin-2-yl)methyl)carbamate

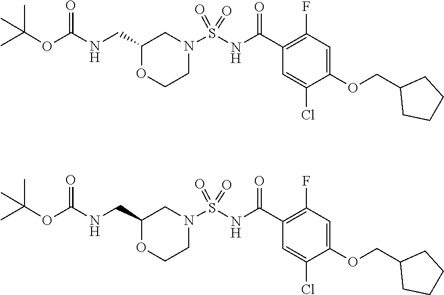 [SMILES: CC(C)(C)OC(=O)NC[C@@H]1CN(S(=O)(=O)NC(=O)c2cc(Cl)c(OCC3CCCC3)cc2F)CCO1 ; CC(C)(C)OC(=O)NC[C@H]1CN(S(=O)(=O)NC(=O)c2cc(Cl)c(OCC3CCCC3)cc2F)CCO1]

Following the procedure described in Example 25 and making non-critical variations as required to replace 4-(cyclopentylmethoxy)-5-cyclopropyl-2-fluorobenzoic acid with 5-chloro-4-(cyclopentylmethoxy)-2-fluorobenzoic acid, tert-butyl ((4-(N-(5-chloro-4-(cyclopentylmethoxy)-2-fluorobenzoyl)sulfamoyl)morpholin-2-yl)methyl)carbamate was obtained as a white solid. tert-butyl ((4-(N-(5-chloro-4-(cyclopentylmethoxy)-2-fluorobenzoyl)sulfamoyl)morpholin-2-yl)methyl)carbamate (220 mg, 0.4 mmol) was separated by using chiral SFC (DAICEL CHIRALCEL OD-H (250 mm*30 mm, 5 um), Supercritical CO₂/EtOH+0.1% NH₃·H₂O=65/35; 60 mL/min) to afford (R)-tert-butyl ((4-(N-(5-chloro-4-(cyclopentylmethoxy)-2-fluorobenzoyl)sulfamoyl)morpholin-2-yl)methyl)carbamate (40 mg, first peak) and (S)-tert-butyl ((4-(N-(5-chloro-4-(cyclopentylmethoxy)-2-fluorobenzoyl)sulfamoyl)morpholin-2-yl)methyl)carbamate (60 mg, second peak) both as a white solid. Absolute configuration was arbitrarily assigned to each enantiomer. LCMS (ESI) m/z: 450.2 [M-100+H]⁺.

### Step 2: (R)-5-chloro-4-(cyclopentylmethoxy)-N-((2-((dimethylamino)methyl)morpholino)sulfonyl)-2-fluorobenzamide

Following the procedure described in Example 9 and making non-critical variations as required to replace tert-butyl 3-((1-(N-(5-cyclopropyl-4-(cyclopropylmethoxy)-2-fluorobenzoyl)sulfamoyl)piperidin-4-yl)oxy)azetidine-1-carboxylate with (R)-tert-butyl ((4-(N-(5-chloro-4-(cyclopentylmethoxy)-2-fluorobenzoyl)sulfamoyl)morpholin-2-yl)methyl)carbamate, the title compound was obtained as a white solid. ¹H NMR (400 MHz, DMSO-d₆) δ 9.42 (s, 1H), 7.74 (d, J=8.0 Hz, 1H), 6.95 (d, J=12.0 Hz, 1H), 3.95 (d, J=6.8 Hz, 2H), 3.94-3.75 (m, 2H), 3.65-3.56 (m, 1H), 3.50-3.35 (m, 4H), 3.10-3.20 (m, 2H), 2.73 (s, 6H), 2.37-2.28 (m, 1H), 1.80-1.72 (m, 2H), 1.66-1.50 (m, 4H), 1.40-1.31 (m, 2H). LCMS (ESI) m/z: 478.0 [M+H]⁺.

### Example 29: (R)-4-(cyclopentylmethoxy)-5-cyclopropyl-2-fluoro-N-((4-(pyrrolidin-3-yloxy)piperidin-1-yl)sulfonyl)benzamide 2,2,2-trifluoroacetate

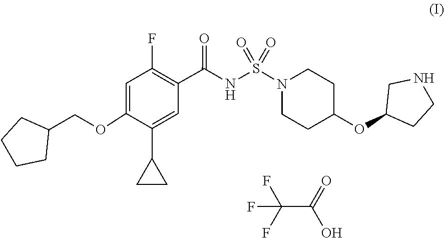 [SMILES: O=C(NS(=O)(=O)N1CCC(O[C@@H]2CCNC2)CC1)c1cc(C2CC2)c(OCC2CCCC2)cc1F ; O=C(O)C(F)(F)F]

### Step 1: (R)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate and (S)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate

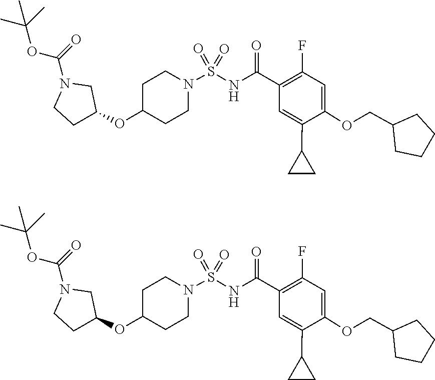 [SMILES: CC(C)(C)OC(=O)N1CC[C@@H](OC2CCN(S(=O)(=O)NC(=O)c3cc(C4CC4)c(OCC4CCCC4)cc3F)CC2)C1 ; CC(C)(C)OC(=O)N1CC[C@H](OC2CCN(S(=O)(=O)NC(=O)c3cc(C4CC4)c(OCC4CCCC4)cc3F)CC2)C1]

Following the procedure described in Example 25 and making non-critical variations as required to replace tert-butyl (morpholin-2-ylmethyl)carbamate with tert-butyl 3-(piperidin-4-yloxy)pyrrolidine-1-carboxylate, tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate was obtained as yellow oil. Tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate (120 mg, 0.23 mmol) was separated by using chiral SFC (Chiralpak AD (250 mm*30 mm, 10 um), Supercritical CO₂/IPA+0.1% NH₃·H₂O=65/35; 70 mL/min) to afford (R)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate (60 mg, first peak) and (S)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate (50 mg, second peak) both as yellow oil. Absolute configuration was arbitrarily assigned to each enantiomer. LCMS (ESI) m/z: 510.3 [M-100+H]⁺.

### Step 2: (R)-4-(cyclopentylmethoxy)-5-cyclopropyl-2-fluoro-N-((4-(pyrrolidin-3-yloxy)piperidin-1-yl)sulfonyl)benzamide 2,2,2-trifluoroacetate

Following the procedure described in Example 1 and making non-critical variations as required to replace tert-butyl 3-((1-(N-(5-chloro-4-(cyclopentylmethoxy)-2-fluorobenzoyl)sulfamoyl)piperidin-4-yl)oxy)azetidine-1-carboxylate with (R)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate, the title compound was obtained as a white solid. ¹H NMR (400 MHz, DMSO-d₆) δ 11.64 (s, 1H), 9.08-8.72 (m, 2H), 7.07 (d, J=8.4 Hz, 1H), 6.94 (d, J=13.2 Hz, 1H), 4.40-4.30 (m, 1H), 3.95 (d, J=6.8 Hz, 2H), 3.63-3.47 (m, 3H), 3.28-3.05 (m, 6H), 2.40-2.27 (m, 1H), 2.06-1.84 (m, 5H), 1.84-1.72 (m, 2H), 1.70-1.43 (m, 6H), 1.42-1.29 (m, 2H), 0.94-0.84 (m, 2H), 0.70-0.61 (m, 2H). LCMS (ESI) m/z: 510.1 [M+H]⁺.

### Example 30: (S)-4-(cyclopentylmethoxy)-5-cyclopropyl-2-fluoro-N-((4-(pyrrolidin-3-yloxy)piperidin-1-yl)sulfonyl)benzamide 2,2,2-trifluoroacetate

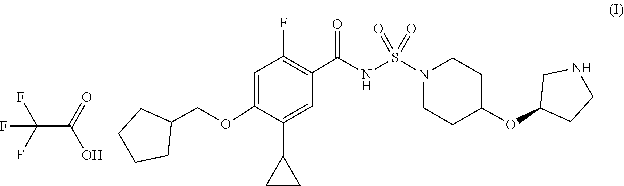

Following the procedure described in Example 29 and making non-critical variations as required to replace (R)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate with (S)-tert-butyl 3-((1-(N-(4-(cyclopentylmethoxy)-5-cyclopropyl-2-fluorobenzoyl)sulfamoyl)piperidin-4-yl)oxy)pyrrolidine-1-carboxylate, the title compound was obtained as a white solid. ¹H NMR (400 MHz, DMSO-d₆) δ 11.64 (s, 1H), 9.02-8.76 (m, 2H), 7.07 (d, J=8.4 Hz, 1H), 6.94 (d, J=13.2 Hz, 1H), 4.40-4.30 (m, 1H), 3.95 (d, J=6.8 Hz, 2H), 3.63-3.47 (m, 3H), 3.28-3.05 (m, 6H), 2.40-2.27 (m, 1H), 2.06-1.84 (m, 5H), 1.84-1.72 (m, 2H), 1.70-1.43 (m, 6H), 1.42-1.29 (m, 2H), 0.94-0.84 (m, 2H), 0.70-0.61 (m, 2H). LCMS (ESI) m/z: 510.1 [M+H]⁺.

### Example 31: (R)-5-chloro-4-(cyclopentylmethoxy)-2-fluoro-N-((3-(pyrrolidin-3-yloxy)azetidin-1-yl)sulfonyl)benzamide 2,2,2-trifluoroacetate

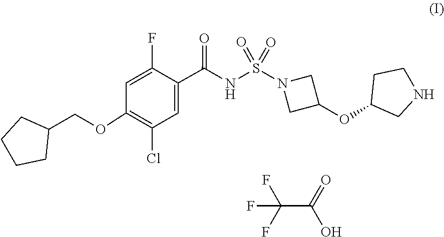 [SMILES: O=C(NS(=O)(=O)N1CC(O[C@@H]2CCNC2)C1)c1cc(Cl)c(OCC2CCCC2)cc1F ; O=C(O)C(F)(F)F]

### Step 1: tert-butyl 3-((1-((benzyloxy)carbonyl)azetidin-3-yl)oxy)pyrrolidine-1-carboxylate

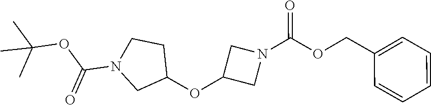 [SMILES: CC(C)(C)OC(=O)N1CCC(OC2CN(C(=O)OCc3ccccc3)C2)C1]

A mixture of benzyl 3-hydroxyazetidine-1-carboxylate (2.73 g, 13.18 mmol) and NaH (527 mg, 13.18 mmol, 60% in mineral oil) in DMF (35 mL) was stirred at 0° C. for 1 h. Tert-butyl 3-(tosyloxy)pyrrolidine-1-carboxylate (3.00 g, 8.79 mmol) was added at 0° C. The mixture was stirred at room temperature for 16 h. The reaction was diluted with water (50 mL) and extracted with DCM (50 mL×2). The combined organic layers were washed with brine (100 mL), dried over anhydrous Na₂SO₄, filtered and concentrated in vacuo. The crude residue was purified by reverse phase chromatography (acetonitrile 25-60%/0.05% NH₃·H₂O+10 mM NH₄HCO₃ in water) to afford the title compound (150 mg, 5%) as a white solid. ¹H NMR (400 MHz, CDCl₃) δ 7.38-7.27 (m, 5H), 5.09 (s, 2H), 4.34-4.28 (m, 1H), 4.21-4.17 (m, 2H), 3.99 (s, 1H), 3.93-3.89 (m, 2H), 3.46-3.33 (m, 4H), 1.93 (s, 2H), 1.46 (s, 9H). LCMS (ESI) m/z: 277.2 [M-100+H]⁺.

### Step 2: tert-butyl 3-(azetidin-3-yloxy)pyrrolidine-1-carboxylate

To a solution of tert-butyl 3-((1-((benzyloxy)carbonyl)azetidin-3-yl)oxy)pyrrolidine-1-carboxylate (150 mg, 0.4 mmol) in EtOAc (9 mL), 10% Pd/C (212 mg, 0.2 mmol) was added. The mixture was stirred at room temperature for 16 h under hydrogen atmosphere (15 psi). The reaction was filtered and concentrated in vacuo to afford the title compound (95 mg, crude) as colorless oil that required no further purification. ¹H NMR (400 MHz, CDCl₃) δ 4.32-4.23 (m, 1H), 3.92 (s, 1H), 3.67-3.52 (m, 3H), 3.35-3.19 (m, 6H), 1.83-1.78 (m, 2H), 1.36 (s, 9H).

### Step 3: (S)-tert-butyl 3-((1-(N-(5-chloro-4-(cyclopentylmethoxy)-2-fluorobenzoyl)-sulfamoyl)azetidin-3-yl)oxy)pyrrolidine-1-carboxylate and (R)-tert-butyl 3-((1-(N-(5-chloro-4-(cyclopentylmethoxy)-2-fluorobenzoyl)sulfamoyl)azetidin-3-yl)oxy)pyrrolidine-1-carboxylate

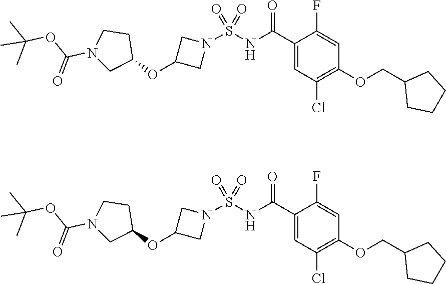 [SMILES: CC(C)(C)OC(=O)N1CC[C@H](OC2CN(S(=O)(=O)NC(=O)c3cc(Cl)c(OCC4CCCC4)cc3F)C2)C1 ; CC(C)(C)OC(=O)N1CC[C@@H](OC2CN(S(=O)(=O)NC(=O)c3cc(Cl)c(OCC4CCCC4)cc3F)C2)C1]

Following the procedure described in Example 19 and making non-critical variations as required to replace 1,2,3,4-tetrahydroisoquinoline with tert-butyl 3-(azetidin-3-yloxy)pyrrolidine-1-carboxylate, tert-butyl 3-((1-(N-(5-chloro-4-(cyclopentylmethoxy)-2-fluorobenzoyl)sulfamoyl)azetidin-3-yl)oxy)pyrrolidine-1-carboxylate was obtained as a white solid. tert-butyl 3-((1-(N-(5-chloro-4-(cyclopentylmethoxy)-2-fluorobenzoyl)sulfamoyl)azetidin-3-yl)oxy)pyrrolidine-1-carboxylate (110 mg, 0.19 mmol) was separated by using chiral SFC (Chiralpak AD (250 mm*30 mm, 10 um), Supercritical NH₃·H₂O/MeOH+Heptane=55/45; 80 mL/min) to afford (S)-tert-butyl 3-((1-(N-(5-chloro-4-(cyclopentylmethoxy)-2-fluorobenzoyl)sulfamoyl)azetidin-3-yl)oxy)pyrrolidine-1-carboxylate (40 mg, first peak) and (R)-tert-butyl 3-((1-(N-(5-chloro-4-(cyclopentylmethoxy)-2-fluorobenzoyl)sulfamoyl)azetidin-3-yl)oxy)pyrrolidine-1-carboxylate (40 mg, second peak) both as a white solid. Absolute configuration was arbitrarily assigned to each enantiomer. LCMS (ESI) m/z: 476.2 [M-100+H]⁺.

### Step 4: (R)-5-chloro-4-(cyclopentylmethoxy)-2-fluoro-N-((3-(pyrrolidin-3-yloxy)azetidin-1-yl)sulfonyl)benzamide 2,2,2-trifluoroacetate

Following the procedure described in Example 1 and making non-critical variations as required to replace tert-butyl 3-((1-(N-(5-chloro-4-(cyclopentylmethoxy)-2-fluorobenzoyl)sulfamoyl)piperidin-4-yl)oxy)azetidine-1-carboxylate with (R)-tert-butyl 3-((1-(N-(5-chloro-4-(cyclopentylmethoxy)-2-fluorobenzoyl)sulfamoyl)azetidin-3-yl)oxy)pyrrolidine-1-carboxylate, the title compound was obtained as a white solid. ¹H NMR (400 MHz, DMSO-d₆) δ 9.06 (s, 2H), 7.76 (d, J=7.6 Hz, 1H), 7.23 (d, J=12.4 Hz, 1H), 4.42-4.36 (m, 1H), 4.24-4.19 (m, 3H), 4.03 (d, J=6.8 Hz, 2H), 3.96-3.92 (m, 2H), 3.24-3.14 (m, 4H), 2.38-2.29 (m, 1H), 1.97-1.92 (m, 2H), 1.81-1.74 (m, 2H), 1.66-1.49 (m, 4H), 1.39-1.31 (m, 2H). LCMS (ESI) m/z: 476.1 [M+H]⁺.

### Example 36: (R)-5-chloro-4-(cyclopentylmethoxy)-2-fluoro-N-((4-(pyrrolidin-3-yloxy)piperidin-1-yl)sulfonyl)benzamide 2,2,2-trifluoroacetate

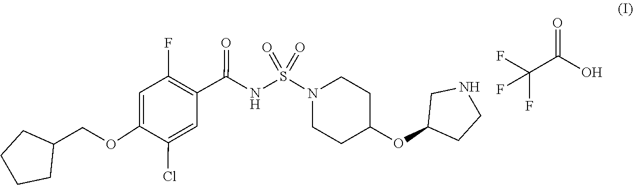

### a. Step 1: (S)-tert-butyl 3-((1-(N-(5-chloro-4-(cyclopentylmethoxy)-2-fluorobenzoyl)sulfamoyl)piperidin-4-yl)oxy)pyrrolidine-1-carboxylate and (R)-tert-butyl 3-((1-(N-(5-chloro-4-(cyclopentylmethoxy)-2-fluorobenzoyl)sulfamoyl)piperidin-4-yl)oxy)pyrrolidine-1-carboxylate

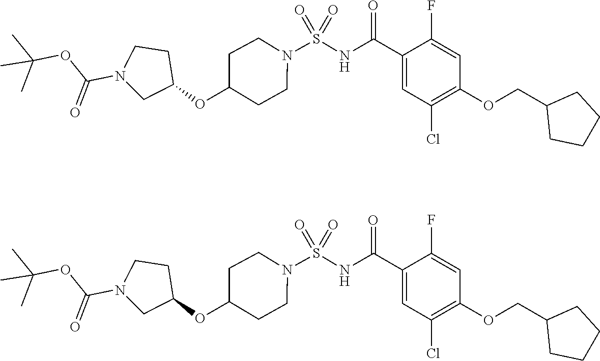 [SMILES: CC(C)(C)OC(=O)N1CC[C@H](OC2CCN(S(=O)(=O)NC(=O)c3cc(Cl)c(OCC4CCCC4)cc3F)CC2)C1 ; CC(C)(C)OC(=O)N1CC[C@@H](OC2CCN(S(=O)(=O)NC(=O)c3cc(Cl)c(OCC4CCCC4)cc3F)CC2)C1]

Following the procedure described in Example 29 and making non-critical variations as required to replace 4-(cyclopentylmethoxy)-5-cyclopropyl-2-fluorobenzoic acid with 5-chloro-4-(cyclopentylmethoxy)-2-fluorobenzoic acid, tert-butyl 3-((1-(N-(5-chloro-4-(cyclopentylmethoxy)-2-fluorobenzoyl)sulfamoyl)piperidin-4-yl)oxy)pyrrolidine-1-carboxylate was obtained as a white solid. Tert-butyl 3-((1-(N-(5-chloro-4-(cyclopentylmethoxy)-2-fluorobenzoyl)sulfamoyl)piperidin-4-yl)oxy)pyrrolidine-1-carboxylate (320 mg, 0.53 mmol) was separated by using chiral SFC (DAICEL CHIRALPAK AD-H (250 mm*30 mm, 5 um), Supercritical CO₂/EtOH+0.1% NH₃·H₂O=65/35; 60 mL/min) to afford (S)-tert-butyl 3-((1-(N-(5-chloro-4-(cyclopentylmethoxy)-2-fluorobenzoyl)sulfamoyl)piperidin-4-yl)oxy)pyrrolidine-1-carboxylate (160 mg, first peak) as a white solid and (R)-tert-butyl 3-((1-(N-(5-chloro-4-(cyclopentylmethoxy)-2-fluorobenzoyl)sulfamoyl)piperidin-4-yl)oxy)pyrrolidine-1-carboxylate (130 mg, second peak) as a white solid. Absolute configuration was arbitrarily assigned to each enantiomer. LCMS (ESI) m/z: 504.2 [M-100+H]⁺.

### Step 2: (R)-5-chloro-4-(cyclopentylmethoxy)-2-fluoro-N-((4-(pyrrolidin-3-yloxy)piperidin-1-yl)sulfonyl)benzamide 2,2,2-trifluoroacetate

Following the procedure described in Example 1 and making non-critical variations as required to replace tert-butyl 3-((1-(N-(5-chloro-4-(cyclopentylmethoxy)-2-fluorobenzoyl)-sulfamoyl)piperidin-4-yl)oxy)azetidine-1-carboxylate with (R)-tert-butyl 3-((1-(N-(5-chloro-4-(cyclopentylmethoxy)-2-fluorobenzoyl)sulfamoyl)piperidin-4-yl)oxy)pyrrolidine-1-carboxylate, the title compound was obtained as a white solid. ¹H NMR (400 MHz, DMSO-d₆) δ 11.81 (s, 1H), 8.99-8.70 (m, 2H), 7.69 (d, J=7.2 Hz, 1H), 7.23 (d, J=12.4 Hz, 1H), 4.38-4.32 (m, 1H), 4.03 (d, J=6.4 Hz, 2H), 3.56-3.51 (m, 2H), 3.24-3.11 (m, 6H), 2.42-2.28 (m, 1H), 2.02-1.93 (m, 2H), 1.92-1.84 (m, 2H), 1.82-1.73 (m, 2H), 1.68-1.59 (m, 2H), 1.58-1.44 (m, 4H), 1.41-1.30 (m, 2H). LCMS (ESI) m/z: 504.2 [M+H]⁺.

### Example 37: (S)-5-chloro-4-(cyclopentylmethoxy)-2-fluoro-N-((4-(pyrrolidin-3-yloxy)-piperidin-1-yl)sulfonyl)benzamide 2,2,2-trifluoroacetate

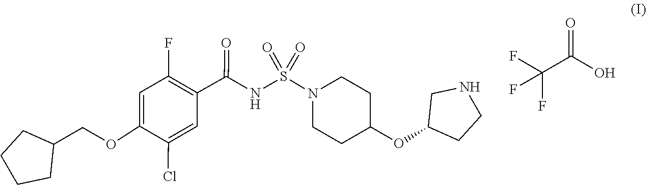

Following the procedure described in Example 36 and making non-critical variations as required to replace (R)-tert-butyl 3-((1-(N-(5-chloro-4-(cyclopentylmethoxy)-2-fluorobenzoyl)sulfamoyl)piperidin-4-yl)oxy)pyrrolidine-1-carboxylate with (S)-tert-butyl 3-((1-(N-(5-chloro-4-(cyclopentylmethoxy)-2-fluorobenzoyl)sulfamoyl)piperidin-4-yl)oxy)pyrrolidine-1-carboxylate, the title compound was obtained as a white solid. ¹H NMR (400 MHz, DMSO-d₆) δ 11.81 (s, 1H), 9.03-8.73 (m, 2H), 7.69 (d, J=7.2 Hz, 1H), 7.23 (d, J=12.4 Hz, 1H), 4.38-4.31 (m, 1H), 4.03 (d, J=6.8 Hz, 2H), 3.60-3.50 (m, 2H), 3.22-3.10 (m, 6H), 2.39-2.29 (m, 1H), 2.01-1.93 (m, 2H), 1.92-1.84 (m, 2H), 1.82-1.73 (m, 2H), 1.66-1.58 (m, 2H), 1.57-1.43 (m, 4H), 1.40-1.30 (m, 2H). LCMS (ESI) m/z: 504.2 [M+H]⁺.

### Example 38: (R)-5-chloro-4-(cyclopentylmethoxy)-2-fluoro-N-((4-((1-methylpyrrolidin-3-yl)oxy)piperidin-1-yl)sulfonyl)benzamide

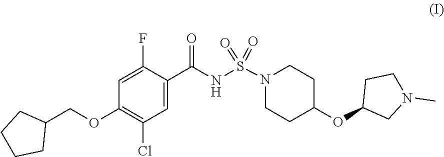 [SMILES: CN1CC[C@H](OC2CCN(S(=O)(=O)NC(=O)c3cc(Cl)c(OCC4CCCC4)cc3F)CC2)C1]

Following the procedure described in Example 1 and making non-critical variations as required to replace N-((4-(azetidin-3-yloxy)piperidin-1-yl)sulfonyl)-5-chloro-4-(cyclopentylmethoxy)-2-fluorobenzamide with (R)-5-chloro-4-(cyclopentylmethoxy)-2-fluoro-N-((4-(pyrrolidin-3-yloxy)piperidin-1-yl)sulfonyl)benzamide 2,2,2-trifluoroacetate, the title compound was obtained as a white solid. ¹H NMR (400 MHz, DMSO-d₆) δ 7.72 (d, J=8.0 Hz, 1H), 6.96 (d, J=12.4 Hz, 1H), 4.35 (m, 1H), 3.96 (d, J=6.8 Hz, 2H), 3.25-3.12 (m, 7H), 2.80-2.70 (m, 5H), 2.36-2.31 (m, 1H), 2.23-2.16 (m, 1H), 1.95-1.85 (m, 3H), 1.79-1.73 (m, 2H), 1.65-1.52 (m, 4H), 1.45-1.33 (m, 4H). LCMS (ESI) m/z: 518.2 [M+H]⁺.

### Example 40: N-((4-(azetidin-3-ylmethyl)piperidin-1-yl)sulfonyl)-5-chloro-4-(cyclopentylmethoxy)-2-fluorobenzamide 2,2,2-trifluoroacetate

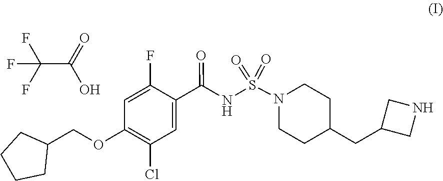

Following the procedure described in Example 1 and making non-critical variations as required to replace tert-butyl 3-(piperidin-4-yloxy)azetidine-1-carboxylate with tert-butyl 3-(piperidin-4-ylmethyl)azetidine-1-carboxylate (Reference: WO2017/95758), the title compound was obtained as a white solid. ¹H NMR (400 MHz, DMSO-d₆) δ 7.68 (d, J=7.6 Hz, 1H), 7.00 (d, J=12.8 Hz, 1H), 4.11 (t, J=10.0 Hz, 2H), 4.01 (d, J=6.4 Hz, 2H), 3.93-3.84 (m, 2H), 3.77 (t, J=8.4 Hz, 2H), 3.11-3.00 (m, 1H), 2.98-2.88 (m, 2H), 2.48-2.38 (m, 1H), 1.92-1.84 (m, 2H), 1.74-1.63 (m, 7H), 1.49-1.38 (m, 3H), 1.34-1.23 (m, 3H). LCMS (ESI) m/z: 488.1 [M+H]⁺.

### Example 41: N-((4-(benzyloxy)piperidin-1-yl)sulfonyl)-5-chloro-4-(cyclopentylmethoxy)-2-fluorobenzamide

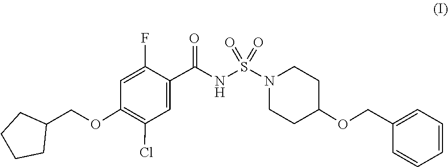 [SMILES: O=C(NS(=O)(=O)N1CCC(OCc2ccccc2)CC1)c1cc(Cl)c(OCC2CCCC2)cc1F]

Following the procedure described in Example 19 and making non-critical variations as required to replace 1,2,3,4-tetrahydroisoquinoline with 4-(benzyloxy)piperidine, the title compound was obtained as a white solid. ¹H NMR (400 MHz, DMSO-d₆) δ 11.83 (s, 1H), 7.72 (d, J=8.0 Hz, 1H), 7.35-7.19 (m, 6H), 4.50 (s, 2H), 4.01 (d, J=6.4 Hz, 2H), 3.57-3.46 (m, 3H), 3.22-3.06 (m, 2H), 2.36-2.30 (m, 1H), 1.94-1.85 (m, 2H), 1.81-1.73 (m, 2H), 1.67-1.57 (m, 4H), 1.56-1.49 (m, 2H), 1.38-1.33 (m, 2H). LCMS (ESI) m/z: 525.2 [M+H]⁺.

### Example 44 and Example 45: 5-chloro-4-(cyclopentylmethoxy)-2-fluoro-N-(((4aR,8aR)-2-oxo-1-phenylhexahydro-1H-pyrido[3,4-b][1,4]oxazin-6(7H)-yl)sulfonyl)benzamide, and 5-chloro-4-(cyclopentylmethoxy)-2-fluoro-N-(((4aS,8aS)-2-oxo-1-phenylhexahydro-1H-pyrido[3,4-b][1,4]oxazin-6(7H)-yl)sulfonyl)benzamide

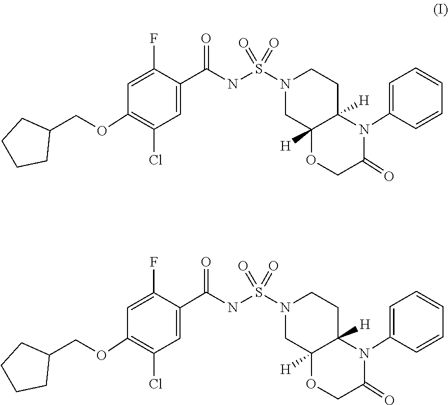

### Step 1: trans-tert-butyl 3-hydroxy-4-(phenylamino)piperidine-1-carboxylate

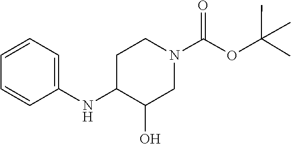 [SMILES: CC(C)(C)OC(=O)N1CCC(Nc2ccccc2)C(O)C1]

To a solution of tert-butyl 7-oxa-3-azabicyclo[4.1.0]heptane-3-carboxylate (2 g, 10.04 mmol) in 1,2-dichloroethane (40 mL) was added aniline (1 g, 11.04 mmol) and tri-(trifluoromethylsulfonyloxy)scandium (0.74 g, 1.51 mmol). The reaction was stirred at room temperature under nitrogen atmosphere for 16 h. The mixture was concentrated in vacuo and the crude residue was purified by silica gel chromatography (solvent gradient: 0-15% EtOAc in petroleum ether) to afford the title compound (800 mg, 27%) as a white solid. LCMS (ESI) m/z: 237.2 [M-56+H]⁺.

### Step 2: trans-tert-butyl 2-oxo-1-phenylhexahydro-1H-pyrido[3,4-b][1,4]oxazine-6(7H)-carboxylate

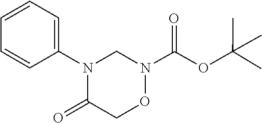 [SMILES: CC(C)(C)OC(=O)N1CN(c2ccccc2)C(=O)CO1]

To a solution of NaH (657 mg, 16.42 mmol, 60% in mineral oil) in THF (15 mL) was added ethyl bromoacetate (0.91 mL, 8.21 mmol) at 0° C. The mixture was stirred for 0.5 h. Trans-tert-butyl 3-hydroxy-4-(phenylamino)piperidine-1-carboxylate (800 mg, 2.74 mmol) was added to the mixture at 0° C. and the reaction was stirred at room temperature for 16 h. The reaction was quenched with saturated aqueous NH₄Cl (20 mL), extracted with EtOAc (30 mL×3). The combined organic layers were washed with brine (50 mL), dried over anhydrous Na₂SO₄, filtered and concentrated in vacuo. The crude residue was purified by silica gel chromatography (solvent gradient: 0-40% EtOAc in petroleum ether) to afford the title compound (450 mg, 50%) as a yellow solid. LCMS (ESI) m/z: 333.2 [M+H]⁺.

### Step 3: 5-chloro-4-(cyclopentylmethoxy)-2-fluoro-N-(((4aR,8aR)-2-oxo-1-phenylhexahydro-1H-pyrido[3,4-b][1,4]oxazin-6(7H)-yl)sulfonyl)benzamide and 5-chloro-4-(cyclopentylmethoxy)-2-fluoro-N-(((4aS,8aS)-2-oxo-1-phenylhexahydro-1H-pyrido[3,4-b][1,4]oxazin-6(7H)-yl)sulfonyl)benzamide

Following the procedure described in Example 42 and making non-critical variations as required to replace trans-1-methylhexahydro-1H-pyrido[3,4-b][1,4]oxazin-2(3H)-one with trans-tert-butyl 2-oxo-1-phenylhexahydro-1H-pyrido[3,4-b][1,4]oxazine-6(7H)-carboxylate, trans-5-chloro-4-(cyclopentylmethoxy)-2-fluoro-N-((2-oxo-1-phenylhexahydro-1H-pyrido[3,4-b][1,4]oxazin-6(7H)-yl)sulfonyl)benzamide was obtained as a yellow solid. Trans-5-chloro-4-(cyclopentylmethoxy)-2-fluoro-N-((2-oxo-1-phenylhexahydro-1H-pyrido[3,4-b][1,4]oxazin-6(7H)-yl)sulfonyl)benzamide (60 mg, 0.09 mmol) was separated by using chiral SFC (Chiralpak AD (250 mm*30 mm, 10 um), Supercritical CO₂/EtOH+0.1% NH₃·H₂O=60/40; 70 mL/min) to afford 5-chloro-4-(cyclopentylmethoxy)-2-fluoro-N-(((4aR,8aR)-2-oxo-1-phenylhexahydro-1H-pyrido[3,4-b][1,4]oxazin-6(7H)-yl)sulfonyl)benzamide (5 mg, second peak) as a white solid. Absolute configuration was arbitrarily assigned. Example 44: ¹H NMR (400 MHz, DMSO-d₆) δ 11.99 (s, 1H), 7.73 (d, J=7.6 Hz, 1H), 7.47-7.37 (m, 2H), 7.36-7.29 (m, 1H), 7.24 (d, J=7.6 Hz, 2H), 7.21-7.24 (m, 1H), 4.45-4.25 (m, 2H), 4.02 (d, J=6.4 Hz, 2H), 3.94-3.72 (m, 3H), 3.67-3.57 (m, 1H), 3.04-2.76 (m, 2H), 2.38-2.25 (m, 1H), 1.85-1.70 (m, 2H), 1.69-1.49 (m, 4H), 1.46-1.27 (m, 4H). LCMS (ESI) m/z: 565.9 [M+H]⁺.

### Example 46: N-((4-(azetidin-3-yloxy)piperidin-1-yl)sulfonyl)-5-chloro-4-(cyclopentyl-methoxy)-2-fluorobenzamide

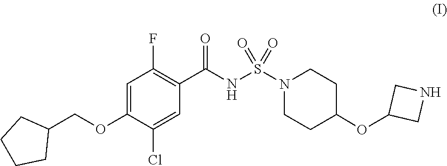 [SMILES: O=C(NS(=O)(=O)N1CCC(OC2CNC2)CC1)c1cc(Cl)c(OCC2CCCC2)cc1F]

Following the procedure described in Example 1 and making non-critical variations as required to replace tert-butyl 3-hydroxyazetidine-1-carboxylate with tert-butyl 3-hydroxyazetidine-1-carboxylate, the title compound was obtained as a white solid. ¹H NMR (400 MHz, DMSO-d₆) δ 7.71 (d, J=7.6 Hz, 1H), 6.92 (d, J=12.4 Hz, 1H), 4.50-4.30 (m, 1H), 4.05-3.96 (m, 2H), 3.96-3.94 (m, 1H), 3.90 (d, J=6.8 Hz, 2H), 3.74-3.67 (m, 2H), 3.26-3.23 (m, 2H), 2.74-2.65 (m, 2H), 2.38-2.27 (m, 1H), 1.85-1.71 (m, 4H), 1.67-1.50 (m, 4H), 1.46-1.29 (m, 4H). LCMS (ESI) m/z: 490.2 [M+H]⁺.

### Example 47: N-((4-(azetidin-3-yloxy)piperidin-1-yl)sulfonyl)-4-(cyclopentylmethoxy)-5-cyclopropyl-2-fluorobenzamide

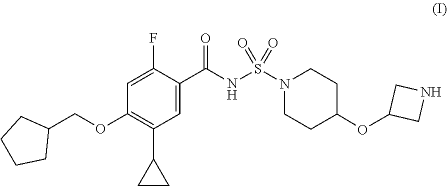 [SMILES: O=C(NS(=O)(=O)N1CCC(OC2CNC2)CC1)c1cc(C2CC2)c(OCC2CCCC2)cc1F]

Following the procedure described in Example 46 and making non-critical variations as required to replace 5-chloro-4-(cyclopentylmethoxy)-2-fluorobenzoic acid with 4-(cyclopentylmethoxy)-5-cyclopropyl-2-fluorobenzoic acid, the title compound was obtained as a white solid. ¹H NMR (400 MHz, DMSO-d₆) δ 7.19 (d, J=8.8 Hz, 1H), 6.64 (d, J=12.4 Hz, 1H), 4.51-4.41 (m, 1H), 4.16-4.07 (m, 2H), 3.87 (d, J=6.8 Hz, 2H), 3.84-3.74 (m, 2H), 3.45-3.37 (m, 4H), 2.78-2.65 (m, 1H), 2.37-2.27 (m, 1H), 2.03-1.94 (m, 1H), 1.84-1.72 (m, 4H), 1.68-1.49 (m, 4H), 1.48-1.31 (m, 4H), 0.90-0.81 (m, 2H), 0.57-0.49 (m, 2H). LCMS (ESI) m/z: 496.1 [M+H]⁺.

### Example 48: (S)-5-chloro-4-(cyclopentylmethoxy)-2-fluoro-N-((3-(piperidin-3-yloxy) azetidin-1-yl) sulfonyl)benzamide

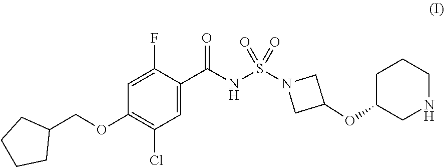 [SMILES: O=C(NS(=O)(=O)N1CC(O[C@@H]2CCCNC2)C1)c1cc(Cl)c(OCC2CCCC2)cc1F]

### Step 1: tert-butyl 3-(piperidin-3-yloxy)azetidine-1-carboxylate

Following the procedure described in Example 1 and making non-critical variations as required to replace tert-butyl 3-hydroxyazetidine-1-carboxylate and pyridin-4-ol with tert-butyl 3-hydroxyazetidine-1-carboxylate and pyridin-3-ol, the title compound was obtained as yellow oil. ¹H NMR (400 MHz, DMSO-d₆) δ 4.34-4.23 (m, 1H), 4.08-3.99 (m, 2H), 3.75-3.84 (m, 2H), 3.29-3.20 (m, 1H), 3.04-2.95 (m, 1H), 2.84-2.75 (m, 1H), 2.65-2.50 (m, 2H), 1.87-1.77 (m, 2H), 1.76-1.65 (m, 1H), 1.47-1.42 (m, 1H), 1.40 (s, 9H).

### Step 2: benzyl 3-((1-(tert-butoxycarbonyl)azetidin-3-yl)oxy)piperidine-1-carboxylate

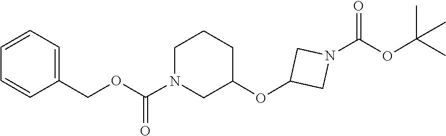 [SMILES: CC(C)(C)OC(=O)N1CC(OC2CCCN(C(=O)OCc3ccccc3)C2)C1]

To a solution of tert-butyl 3-(piperidin-3-yloxy)azetidine-1-carboxylate (500 mg, 1.95 mmol) in DCM (15 mL) was added DIPEA (0.97 mL, 5.85 mmol) and benzyl chloroformate (0.4 mL, 2.93 mmol). The mixture was stirred at room temperature for 16 h. The reaction was quenched with saturated aqueous NaHCO₃(20 mL) and extracted with DCM (50 mL×2). The combined organic layers were washed with brine (30 mL), dried over anhydrous Na₂SO₄, filtered and concentrated in vacuo to afford the title compound (620 mg, crude) as yellow oil that required no further purification. LCMS (ESI) m/z: 291.2 [M-100+H]⁺.

### Step 3: benzyl 3-(azetidin-3-yloxy)piperidine-1-carboxylate

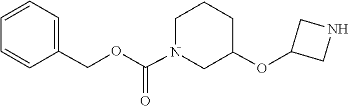 [SMILES: O=C(OCc1ccccc1)N1CCCC(OC2CNC2)C1]

Following the procedure described in Example 2 and making non-critical variations as required to replace tert-butyl 4-(cyclopentylmethoxy)-2-fluoro-5-methylbenzoate with benzyl 3-((1-(tert-butoxycarbonyl)azetidin-3-yl)oxy)piperidine-1-carboxylate, the title compound was obtained as yellow oil. LCMS (ESI) m/z: 291.2 [M+H]⁺.

### Step 4: benzyl 3-((1-(N-(5-chloro-4-(cyclopentylmethoxy)-2-fluorobenzoyl)sulfamoyl)azetidin-3-yl)oxy)piperidine-1-carboxylate

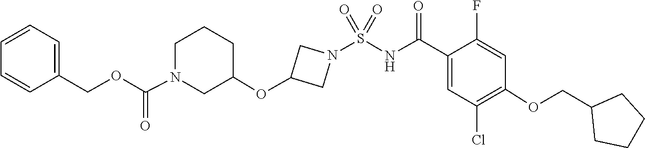 [SMILES: O=C(NS(=O)(=O)N1CC(OC2CCCN(C(=O)OCc3ccccc3)C2)C1)c1cc(Cl)c(OCC2CCCC2)cc1F]

Following the procedure described in Example 1 and making non-critical variations as required to replace tert-butyl 3-(piperidin-4-yloxy)azetidine-1-carboxylate with benzyl 3-(azetidin-3-yloxy)piperidine-1-carboxylate, the title compound was obtained as yellow oil. LCMS (ESI) m/z: 624.1 [M+H]⁺.

### Step 5: (S)-benzyl 3-((1-(N-(5-chloro-4-(cyclopentylmethoxy)-2-fluorobenzoyl)-sulfamoyl)azetidin-3-yl)oxy)piperidine-1-carboxylate, and (R)-benzyl 3-((1-(N-(5-chloro-4-(cyclopentylmethoxy)-2-fluorobenzoyl)sulfamoyl)-azetidin-3-yl)oxy)piperidine-1-carboxylate

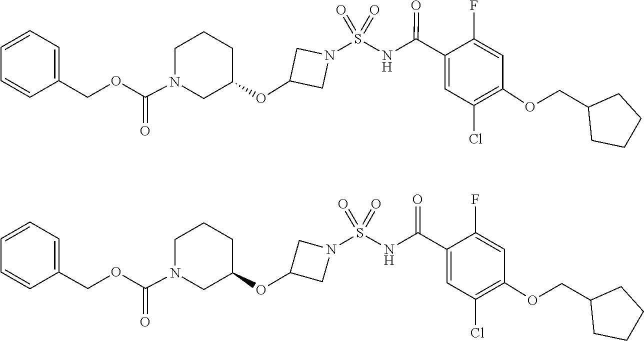 [SMILES: O=C(NS(=O)(=O)N1CC(O[C@H]2CCCN(C(=O)OCc3ccccc3)C2)C1)c1cc(Cl)c(OCC2CCCC2)cc1F ; O=C(NS(=O)(=O)N1CC(O[C@@H]2CCCN(C(=O)OCc3ccccc3)C2)C1)c1cc(Cl)c(OCC2CCCC2)cc1F]

benzyl 3-((1-(N-(5-chloro-4-(cyclopentylmethoxy)-2-fluorobenzoyl)sulfamoyl)azetidin-3-yl)oxy)piperidine-1-carboxylate (150 mg, 0.24 mmol) was separated by using chiral SFC daicel chiralpak IG (250 mm*30 mm, 10 um; Supercritical CO₂/i-PrOH+0.1% NH₃·H₂O=50/50; 80 mL/min) to afford (S)-benzyl 3-((1-(N-(5-chloro-4-(cyclopentylmethoxy)-2-fluorobenzoyl)sulfamoyl)azetidin-3-yl)oxy)piperidine-1-carboxylate (70 mg, first peak) and (R)-benzyl 3-((1-(N-(5-chloro-4-(cyclopentylmethoxy)-2-fluorobenzoyl)sulfamoyl)azetidin-3-yl)oxy)piperidine-1-carboxylate (70 mg, second peak) both as a white solid. Absolute configuration was arbitrarily assigned to each enantiomer. LCMS (ESI) m/z: 624.1 [M+H]⁺.

### Step 6: (S)-5-chloro-4-(cyclopentylmethoxy)-2-fluoro-N-((3-(piperidin-3-yloxy)azetidin-1-yl)sulfonyl)benzamide

A solution of (S)-benzyl 3-((1-(N-(5-chloro-4-(cyclopentylmethoxy)-2-fluorobenzoyl)-sulfamoyl)azetidin-3-yl)oxy)piperidine-1-carboxylate (70 mg, 0.11 mmol) in DCM (1 mL) was added PdCl₂ (5 mg, 0.025 mmol). The mixture was stirred for 16 h under hydrogen atmosphere (15 psi) at room temperature. The resulting residue was purified by reverse phase chromatography (acetonitrile 25-55%/0.2% formic acid in water) to afford the title compound (21 mg, 39%) as a white solid. ¹H NMR (400 MHz, DMSO-d₆) δ 7.79 (d, J=8.0 Hz, 1H), 6.95 (d, J=12.4 Hz, 1H), 4.34-4.24 (m, 1H), 3.95 (d, J=6.8 Hz, 2H), 3.84-3.75 (m, 2H), 3.72-3.56 (m, 3H), 3.18-3.08 (m, 1H), 2.99-2.85 (m, 3H), 2.36-2.27 (m, 1H), 1.81-1.73 (m, 4H), 1.63-1.52 (m, 6H), 1.41-1.31 (m, 2H). LCMS (ESI) m/z: 490.1 [M+H]⁺.

### Example 49: (R)-5-chloro-4-(cyclopentylmethoxy)-2-fluoro-N-((3-(piperidin-3-yloxy)-azetidin-1-yl)sulfonyl)benzamide

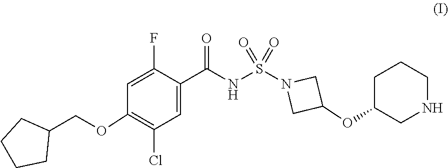 [SMILES: O=C(NS(=O)(=O)N1CC(O[C@@H]2CCCNC2)C1)c1cc(Cl)c(OCC2CCCC2)cc1F]

Following the procedure described in Example 48 and making non-critical variations as required to replace (S)-benzyl 3-((1-(N-(5-chloro-4-(cyclopentylmethoxy)-2-fluorobenzoyl)sulfamoyl)azetidin-3-yl)oxy)piperidine-1-carboxylate with (R)-benzyl 3-((1-(N-(5-chloro-4-(cyclopentylmethoxy)-2-fluorobenzoyl)sulfamoyl)azetidin-3-yl)oxy)piperidine-1-carboxylate, the title compound was obtained as a white solid. ¹H NMR (400 MHz, DMSO-d₆) δ 7.78 (d, J=8.0 Hz, 1H), 6.95 (d, J=12.4 Hz, 1H), 4.32-4.23 (m, 1H), 3.95 (d, J=6.8 Hz, 2H), 3.83-3.75 (m, 2H), 3.72-3.62 (m, 3H), 3.16-3.09 (m, 1H), 2.99-2.85 (m, 3H), 2.36-2.27 (m, 1H), 1.81-1.74 (m, 4H), 1.64-1.51 (m, 6H), 1.41-1.30 (m, 2H). LCMS (ESI) m/z: 490.0 [M+H]⁺.

### Example 50: 5-chloro-4-(cyclopentylmethoxy)-2-fluoro-N-((3-(piperidin-4-yloxy) azetidin-1-yl)sulfonyl)benzamide

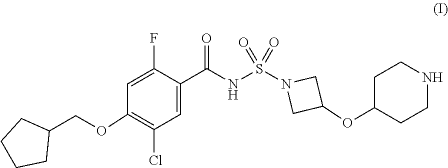 [SMILES: O=C(NS(=O)(=O)N1CC(OC2CCNCC2)C1)c1cc(Cl)c(OCC2CCCC2)cc1F]

Following the procedure described in Example 31 and making non-critical variations as required to replace tert-butyl 3-hydroxypyrrolidine-1-carboxylate with tert-butyl 4-hydroxypiperidine-1-carboxylate, the title compound was obtained as a white solid. ¹H NMR (400 MHz, DMSO-d₆) δ 7.78 (d, J=8.0 Hz, 1H), 6.94 (d, J=12.4 Hz, 1H), 4.26-4.18 (m, 1H), 3.95 (d, J=6.8 Hz, 2H), 3.79-3.73 (m, 2H), 3.67-3.61 (m, 2H), 3.30-3.25 (m, 1H), 3.19-3.14 (m, 1H), 2.97-2.88 (m, 2H), 2.37-2.27 (m, 1H), 1.94-1.85 (m, 2H), 1.82-1.75 (m, 2H), 1.66-1.52 (m, 6H), 1.40-1.33 (m, 2H), LCMS (ESI) m/z: 490.1 [M+H]⁺.

### Example 51: 5-chloro-4-(cyclopentylmethoxy)-2-fluoro-N-((4-(piperidin-3-yloxy)piperidin-1-yl)sulfonyl)benzamide 2,2,2-trifluoroacetate

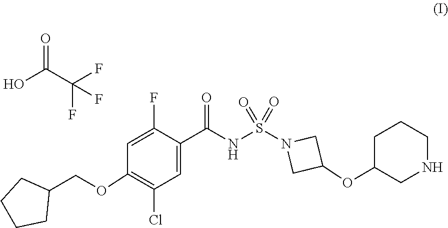 [SMILES: O=C(NS(=O)(=O)N1CC(OC2CCCNC2)C1)c1cc(Cl)c(OCC2CCCC2)cc1F ; O=C(O)C(F)(F)F]

Following the procedure described in Example 1 and making non-critical variations as required to replace tert-butyl 3-hydroxyazetidine-1-carboxylate with tert-butyl 3-hydroxy-piperidine-1-carboxylate, the title compound was obtained as a white solid, assumed to be a mixture of enantiomers. ¹H NMR (400 MHz, DMSO-d₆) δ 11.83 (s, 1H), 8.55 (s, 1H), 8.27 (s, 1H), 7.69 (d, J=7.2 Hz, 1H), 7.23 (d, J=12.4 Hz, 1H), 4.03 (d, J=6.8 Hz, 2H), 3.80-3.70 (m, 1H), 3.69-3.60 (m, 1H), 3.59-3.45 (m, 2H), 3.22-3.05 (m, 3H), 2.99-2.90 (m, 3H), 2.40-2.25 (m, 1H), 1.93-1.70 (m, 6H), 1.68-1.57 (m, 4H), 1.57-1.45 (m, 4H), 1.43-1.29 (m, 2H), LCMS (ESI) m/z: 518.1 [M+H]⁺.

### Example 52: 5-chloro-4-(cyclopentylmethoxy)-2-fluoro-N-((4-((1-methylpiperidin-3-yl)oxy)piperidin-1-yl)sulfonyl) benzamide

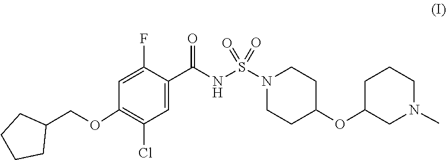 [SMILES: CN1CCCC(OC2CCN(S(=O)(=O)NC(=O)c3cc(Cl)c(OCC4CCCC4)cc3F)CC2)C1]

Following the procedure described in Example 1 and making non-critical variations as required to replace N-((4-(azetidin-3-yloxy)piperidin-1-yl)sulfonyl)-5-chloro-4-(cyclopentylmethoxy)-2-fluorobenzamide with 5-chloro-4-(cyclopentylmethoxy)-2-fluoro-N-((4-(piperidin-3-yloxy)piperidin-1-yl)sulfonyl)benzamide, the title compound was obtained as a white solid. ¹H NMR (400 MHz, DMSO-d₆) δ 7.72 (d, J=8.0 Hz, 1H), 6.96 (d, J=12.0 Hz, 1H), 3.95 (d, J=6.8 Hz, 2H), 3.75-3.68 (m, 1H), 3.60-3.40 (m, 3H), 3.21-3.05 (m, 2H), 2.97-2.89 (m, 1H), 2.85-2.72 (m, 3H), 2.60 (s, 3H), 2.37-2.25 (m, 1H), 1.89-1.69 (m, 6H), 1.67-1.49 (m, 6H), 1.48-1.26 (m, 4H), LCMS (ESI) m/z: 532.1 [M+H]⁺.

### Example 53: 5-chloro-4-(cyclopentylmethoxy)-2-fluoro-N-((4-(piperidin-4-yloxy) piperidin-1-yl)sulfonyl) benzamide

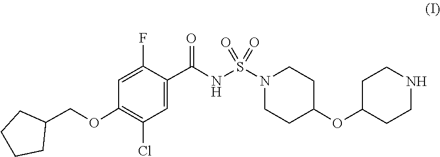 [SMILES: O=C(NS(=O)(=O)N1CCC(OC2CCNCC2)CC1)c1cc(Cl)c(OCC2CCCC2)cc1F]

Following the procedure described in Example 1 and making non-critical variations as required to replace tert-butyl 3-hydroxyazetidine-1-carboxylate with tert-butyl 4-(piperidin-4-yloxy) piperidine-1-carboxylate, the title compound was obtained as a yellow solid. ¹H NMR (400 MHz, DMSO-d₆) δ 8.35 (s, 1H), 7.71 (d, J=8.0 Hz, 1H), 7.06 (d, J=12.0 Hz, 1H), 3.98 (d, J=6.4 Hz, 2H), 3.78-3.62 (m, 1H), 3.61-3.48 (m, 1H), 3.46-3.40 (m, 2H), 3.21-3.08 (m, 2H), 3.03-2.86 (m, 4H), 2.36-2.29 (m, 1H), 1.96-1.86 (m, 2H), 1.85-1.76 (m, 4H), 1.66-1.57 (m, 4H), 1.56-1.52 (m, 2H), 1.49-1.40 (m, 2H), 1.35-1.25 (m, 2H). LCMS (ESI) m/z: 518.2 [M+H]⁺.

### Example 57: 3-chloro-4-(cyclopentylmethoxy)-N-((4-(((1S,2S)-2-(dimethylamino)-cyclohexyl)amino)-2-fluorophenyl)sulfonyl)benzamide formate

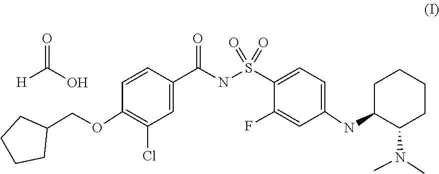

### Step 1: 3-chloro-4-(cyclopentylmethoxy)-N-((2,4-difluorophenyl)sulfonyl)benzamide

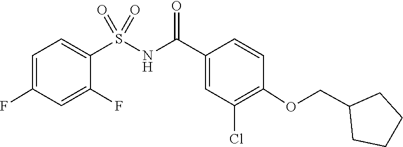 [SMILES: O=C(NS(=O)(=O)c1ccc(F)cc1F)c1ccc(OCC2CCCC2)c(Cl)c1]

To a mixture of DMAP (126 mg, 1.04 mmol) and EDCI (99 mg, 0.52 mmol) in DCM (4 mL) was added 3-chloro-4-(cyclopentylmethoxy)benzoic acid (132 mg, 0.52 mmol) and 2,4-difluorobenzenesulfonamide (100 mg, 0.52 mmol). The mixture was stirred at room temperature for 16 h under nitrogen atmosphere. The reaction was diluted with water (10 mL) and extracted with EtOAc (15 mL×3). The combined organic layers were washed with brine (50 mL), dried over anhydrous Na₂SO₄, filtered and concentrated in vacuo. The crude residue was purified by prep-TLC (DCM:MeOH=20:1) to afford the title compound (180 mg, 0.42 mmol) as colorless oil. LCMS (ESI) m/z: 430.0 [M+H]⁺.

### Step 2: 3-chloro-4-(cyclopentylmethoxy)-N-((4-(((1S,2S)-2-(dimethylamino)-cyclohexyl)amino)-2-fluorophenyl)sulfonyl)benzamide formate

Following the procedure described in Example 55 and making non-critical variations as required to replace N-((5-chloro-2,4-difluorophenyl)sulfonyl)-4-(cyclopentylmethoxy)-5-cyclopropyl-2-fluorobenzamide with 3-chloro-4-(cyclopentylmethoxy)-N-((2,4-difluorophenyl)sulfonyl)benzamide, the title compound was obtained as a yellow solid. ¹H NMR (400 MHz, DMSO-d₆) δ 12.48 (s, 1H), 8.78 (s, 1H), 7.98 (s, 1H), 7.90-7.80 (m, 1H), 7.65 (t, J=8.8 Hz, 1H), 7.23 (d, J=8.8 Hz, 1H), 6.77 (s, 1H), 6.69-6.50 (m, 2H), 4.02 (d, J=6.8 Hz, 2H), 3.90-3.75 (m, 1H), 3.20-3.09 (m, 1H), 2.76 (d, J=4.4 Hz, 3H), 2.59 (d, J=4.8 Hz, 3H), 2.40-2.30 (m, 1H), 2.11-2.01 (m, 1H), 2.00-1.91 (m, 1H), 1.87-1.72 (m, 3H), 1.67-1.50 (m, 5H), 1.46-1.25 (m, 5H), 1.21-1.08 (m, 1H). LCMS (ESI) m/z: 552.1 [M+H]⁺.

### Example 58: 5-chloro-4-(cyclopentylmethoxy)-N-((2,6-difluoro-4-(piperidin-1-yl)phenyl)-sulfonyl)-2-fluorobenzamide

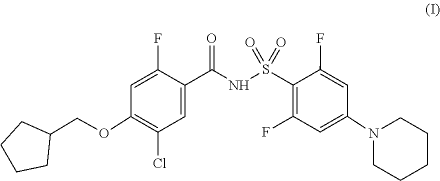 [SMILES: O=C(NS(=O)(=O)c1c(F)cc(N2CCCCC2)cc1F)c1cc(Cl)c(OCC2CCCC2)cc1F]

Following the procedure described in Example 57 and making non-critical variations as required to replace 2,4-difluorobenzenesulfonamide with 2,4,6-trifluorobenzenesulfonamide, replace 3-chloro-4-(cyclopentylmethoxy)benzoic acid with 5-chloro-4-(cyclopentylmethoxy)-2-fluorobenzoic acid, and replace (1S,2S)—N1,N1-dimethylcyclohexane-1,2-diamine with piperidine, the title compound was obtained as a white solid. ¹H NMR (400 MHz, DMSO-d₆) δ 7.66 (d, J=7.6 Hz, 1H), 7.21 (d, J=12.6 Hz, 1H), 6.70 (d, J=14.0 Hz, 2H), 4.02 (d, J=6.8 Hz, 2H), 3.42-3.39 (m, 4H), 2.37-2.27 (m, 1H), 1.83-1.73 (m, 2H), 1.64-1.48 (m, 1OH), 1.40-1.30 (m, 2H). LCMS (ESI) m/z: 531.2 [M+H]⁺.

### Example 59: 5-chloro-4-(cyclopentylmethoxy)-N-((4-(((1S,2S)-2-(dimethylamino)-cyclohexyl)amino)-2-fluorophenyl)sulfonyl)-2-fluorobenzamide

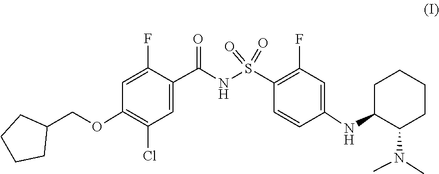 [SMILES: CN(C)[C@H]1CCCC[C@@H]1Nc1ccc(S(=O)(=O)NC(=O)c2cc(Cl)c(OCC3CCCC3)cc2F)c(F)c1]

### Step 1: 4-(((1S,2S)-2-(dimethylamino)cyclohexyl)amino)-2-fluorobenzenesulfonamide

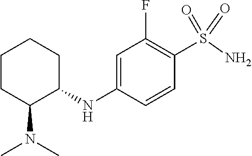 [SMILES: CN(C)[C@H]1CCCC[C@@H]1Nc1ccc(S(N)(=O)=O)c(F)c1]

A solution of (1S,2S)—N¹,N¹-dimethylcyclohexane-1,2-diamine (162 mg, 1.1 mmol), 2,4-difluorobenzenesulfonamide (200 mg, 1.0 mmol) and DIPEA (0.3 mL, 1.7 mmol) in DMSO (3 mL) was stirred at 80° C. for 16 h. After cooling to room temperature, the reaction was diluted with EtOAc (100 mL), and washed with brine (50 mL×5). The organic lawyer was dried over anhydrous Na₂SO₄, filtered and concentrated in vacuo. The crude residue was purified by silica gel chromatography (solvent gradient: 0-10% EtOAc in petroleum ether) to afford the title compound (60 mg, 0.19 mmol) as yellow oil. LCMS (ESI) m/z: 316.1 [M+H]⁺.

### Step 2: 5-chloro-4-(cyclopentylmethoxy)-N-((4-(((1S,2S)-2-(dimethylamino)-cyclohexyl)amino)-2-fluorophenyl)sulfonyl)-2-fluorobenzamide

To a solution of 4-(((1S,2S)-2-(dimethylamino)cyclohexyl)amino)-2-fluorobenzenesulfonamide (80 mg, 0.24 mmol) and DMAP (59 mg, 0.48 mmol) in DCM (2 mL) was added EDCI (51 mg, 0.26 mmol) and 5-chloro-4-(cyclopentylmethoxy)-2-fluoro-benzoic acid (72 mg, 0.26 mmol). The reaction was stirred at room temperature for 2 h. The reaction was quenched with 10% aqueous citric acid (5 mL). The reaction was diluted with water (10 mL) and extracted with DCM (10 mL×3). The combined organic layers were dried over anhydrous Na₂SO₄, filtered and concentrated in vacuo. The crude residue was purified by silica gel chromatography (solvent gradient: 0-5% MeOH in DCM) to afford the title compound (40 mg, 28%) as a white solid. ¹H NMR (400 MHz, DMSO-d₆) δ 7.75 (d, J=7.6 Hz, 1H), 7.55-7.48 (m, 1H), 6.94 (d, J=12.4 Hz, 1H), 6.50 (s, 1H), 6.47 (s, 1H), 6.07 (d, J=10.0 Hz, 1H), 3.94 (d, J=6.8 Hz, 2H), 3.79-3.64 (m, 1H), 3.15-3.05 (m, 1H), 2.66 (s, 6H), 2.35-2.25 (m, 1H), 2.09-1.94 (m, 2H), 1.84-1.69 (m, 3H), 1.63-1.14 (m, 11H). LCMS (ESI) m/z: 570.2 [M+H]⁺.

### Example 62: 5-chloro-4-(cyclopentylmethoxy)-N-((4-(3-(dimethylamino)piperidin-1-yl)-2,6-difluorophenyl)sulfonyl)-2-fluorobenzamide formate

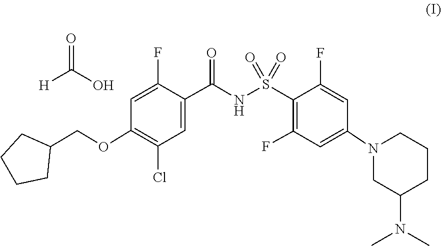

### Step 1: 4-(3-(dimethylamino)piperidin-1-yl)-2,6-difluorobenzenesulfonamide

A solution of 3-dimethylaminopiperidine (35 mg, 0.27 mmol), 2,4,6-trifluoro-benzenesulfonamide (57 mg, 0.27 mmol) and DIPEA (0.36 mL, 2.18 mmol) in DMSO (1 mL) was stirred at 40° C. for 16 h. After cooling to room temperature, the reaction was diluted with EtOAc (60 mL), and washed with brine (30 mL×5). The organic lawyer was dried over anhydrous Na₂SO₄, filtered and concentrated in vacuo. The crude residue was purified by prep-TLC (DCM:MeOH=10:1) to afford the title compound (20 mg, 16%) as a white solid. LCMS (ESI) m/z: 320.1 [M+H]⁺.

### Step 2: 5-chloro-4-(cyclopentylmethoxy)-N-((4-(3-(dimethylamino)piperidin-1-yl)-2,6-difluorophenyl)sulfonyl)-2-fluorobenzamide formate

Following the procedure described in Example 59 and making non-critical variations as required to replace 4-(((1S,2S)-2-(dimethylamino)cyclohexyl)amino)-2-fluorobenzene-sulfonamide with 4-(3-(dimethylamino)piperidin-1-yl)-2,6-difluorobenzenesulfonamide and replace (1S,2S)—N1,N1-dimethylcyclohexane-1,2-diamine with N,N-dimethylpiperidin-3-amine, the title compound was obtained as a white solid. ¹H NMR (400 MHz, DMSO-d₆) δ 7.75 (d, J=8.0 Hz, 1H), 6.95 (d, J=12.4 Hz, 1H), 6.57 (d, J=12.4 Hz, 2H), 3.95 (d, J=6.4 Hz, 3H), 3.78-3.73 (m, 1H), 3.13-2.94 (m, 2H), 2.86-2.75 (m, 8H), 2.35-2.28 (m, 1H), 2.07-1.95 (m, 1H), 1.78-1.72 (m, 3H), 1.61-1.51 (m, 6H), 1.39-1.30 (m, 2H). LCMS (ESI) m/z: 574.3 [M+H]⁺.

### Example 63: 4-(cyclopentylmethoxy)-5-cyclopropyl-N-((4-(((1S,2S)-2-(dimethylamino)-cyclohexyl)amino)-2-fluorophenyl)sulfonyl)-2-fluorobenzamide

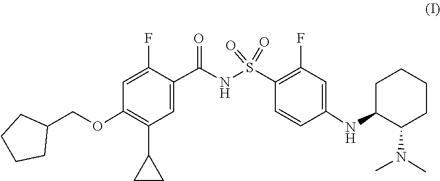 [SMILES: CN(C)[C@H]1CCCC[C@@H]1Nc1ccc(S(=O)(=O)NC(=O)c2cc(C3CC3)c(OCC3CCCC3)cc2F)c(F)c1]

Following the procedure described in Example 59 and replace 5-chloro-4-(cyclopentylmethoxy)-2-fluorobenzoic acid with 4-(cyclopentylmethoxy)-5-cyclopropyl-2-fluorobenzoic acid, the title compound was obtained as a white solid. ¹H NMR (400 MHz, DMSO-d₆) δ 7.50 (t, J=8.8 Hz, 1H), 7.20 (d, J=8.8 Hz, 1H), 6.65 (d, J=12.0 Hz, 1H), 6.52-6.34 (m, 2H), 5.99 (s, 1H), 3.87 (d, J=6.8 Hz, 2H), 3.72-3.52 (m, 1H), 3.05-2.81 (m, 1H), 2.61-2.52 (m, 6H), 2.37-2.26 (m, 1H), 2.07-1.90 (m, 3H), 1.87-1.71 (m, 3H), 1.66-1.52 (m, 5H), 1.45-1.30 (m, 4H), 1.29-1.20 (m, 1H), 1.15-1.05 (m, 1H), 0.91-0.80 (m, 2H), 0.60-0.45 (m, 2H). LCMS (ESI) m/z: 576.1[M+H]⁺.

### Example 64: 5-chloro-4-(cyclopentylmethoxy)-N-((4-(((1S,2S)-2-(dimethylamino)-cyclohexyl)amino)phenyl)sulfonyl)-2-fluorobenzamide formate

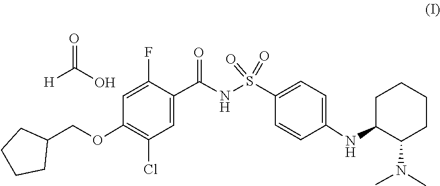

### Step 1: 4-(((1S,2S)-2-(dimethylamino)cyclohexyl)amino)benzenesulfonamide

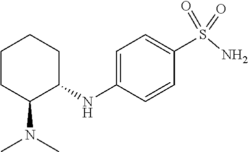 [SMILES: CN(C)[C@H]1CCCC[C@@H]1Nc1ccc(S(N)(=O)=O)cc1]

A solution of (1S,2S)—N1,N1-dimethylcyclohexane-1,2-diamine (811 mg, 5.71 mmol), 4-fluorobenzenesulfonamide (200 mg, 1.14 mmol) and DIPEA (737 mg, 5.71 mmol) in DMSO (1 mL) was stirred at 150° C. for 2 h under microwave. After cooling to room temperature, the reaction was diluted with EtOAc (60 mL), and washed with brine (30 mL×5). The organic lawyer was dried over anhydrous Na₂SO₄, filtered and concentrated in vacuo. The crude residue was purified by silica gel chromatography (solvent gradient: 0-50% EtOAc in petroleum ether) to afford the title compound (125 mg, 15%) as a white solid. LCMS (ESI) m/z: 298.2 [M+H]⁺.

### Step 2: 5-chloro-4-(cyclopentylmethoxy)-N-((4-(((1S,2S)-2-(dimethylamino)-cyclohexyl)amino)phenyl)sulfonyl)-2-fluorobenzamide formate

Following the procedure described in Example 59 and making non-critical variations as required to replace 4-(((1S,2S)-2-(dimethylamino)cyclohexyl)amino)-2-fluorobenzene-sulfonamide with 4-(((1S,2S)-2-(dimethylamino)cyclohexyl)amino) benzenesulfonamide as a white solid. ¹H NMR (400 MHz, DMSO-d₆) δ 8.27 (s, 1H), 7.71 (d, J=8.0 Hz, 1H), 7.58 (d, J=8.8 Hz, 2H), 6.92 (d, J=12.4 Hz, 1H), 6.68 (d, J=8.8 Hz, 2H), 5.77 (s, 1H), 3.93 (d, J=6.8 Hz, 2H), 3.70-3.58 (m, 1H), 3.12-3.07 (m, 1H), 2.63 (s, 6H), 2.34-2.26 (m, 1H), 2.03-1.93 (m, 2H), 1.92-1.68 (m, 3H), 1.67-1.39 (m, 6H), 1.38-1.21 (m, 4H), 1.19-1.07 (m, 1H). LCMS (ESI) m/z: 552.3 [M+H]⁺.

### Example 65: 4-(cyclopentylmethoxy)-5-cyclopropyl-N-((4-(((1S,2S)-2-(dimethylamino)cyclohexyl)oxy)-2-fluorophenyl)sulfonyl)-2-fluorobenzamide

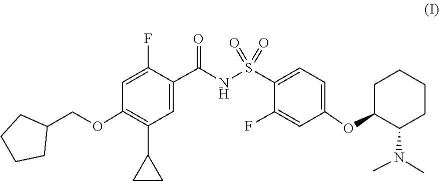 [SMILES: CN(C)[C@H]1CCCC[C@@H]1Oc1ccc(S(=O)(=O)NC(=O)c2cc(C3CC3)c(OCC3CCCC3)cc2F)c(F)c1]

### Step 1: 4-(((1S,2S)-2-(dimethylamino)cyclohexyl)oxy)-2-fluorobenzenesulfonamide

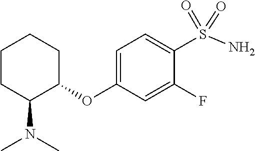 [SMILES: CN(C)[C@H]1CCCC[C@@H]1Oc1ccc(S(N)(=O)=O)c(F)c1]

To a solution of thionylchloride (0.07 mL, 0.95 mmol) in DCM (2 mL) at 0° C., 4-[(1S,2S)-2-(dimethylamino)cyclohexoxy]-2-fluoro-benzenesulfonic acid (100 mg, 0.32 mmol) and two drops of DMF were added. The mixture was stirred at room temperature for 2 h. Then NH₃·H₂O (0.1 mL) was added at 0° C. and the reaction was stirred at room temperature for 30 min. The solvent was concentrated in vacuo to afford the title compound (126 mg, crude) as colorless oil that required no further purification. LCMS (ESI) m/z: 317.2 [M+H]⁺.

### Step 2: 4-(cyclopentylmethoxy)-5-cyclopropyl-N-((4-(((1S,2S)-2-(dimethylamino)cyclohexyl)oxy)-2-fluorophenyl)sulfonyl)-2-fluorobenzamide

To a solution of 4-(((1S,2S)-2-(dimethylamino)cyclohexyl)oxy)-2-fluorobenzenesulfonamide in DCM (2 mL) was added HATU (35 mg, 0.09 mmol), DIPEA (0.06 mL, 0.36 mmol) and 4-(cyclopentylmethoxy)-5-cyclopropyl-2-fluoro-benzoic acid (20 mg, 0.07 mmol). The mixture was stirred at room temperature for 16 h. The reaction was diluted with water (30 mL), and extracted with EtOAc (30 mL×3). The combined organic layers were washed with brine (50 mL), dried over anhydrous Na₂SO₄, filtered and concentrated in vacuo. The crude residue was purified by reverse phase chromatography (acetonitrile 33-63%/0.2% HCOOH in water) to afford the title compound (3 mg, 7%) as a white solid. ¹H NMR (400 MHz, DMSO-d₆) δ 7.80-7.68 (m, 1H), 7.23 (d, J=8.4 Hz, 1H), 6.96-6.82 (m, 2H), 6.65 (d, J=11.6 Hz, 1H), 4.75-4.60 (m, 1H), 3.87 (d, J=6.8 Hz, 2H), 3.25-3.15 (m, 1H), 2.67 (s, 6H), 2.36-2.29 (m, 1H), 2.25-2.12 (m, 1H), 2.06-1.92 (m, 2H), 1.86-1.73 (m, 2H), 1.71-1.23 (m, 12H), 0.90-0.80 (m, 2H), 0.57-0.48 (m, 2H). LCMS (ESI) m/z: 577.1 [M+H]⁺.

### Example 66: 5-chloro-4-(cyclopentylmethoxy)-N-((4-(((1S,2S)-2-(dimethylamino)-cyclohexyl)oxy)-2-fluorophenyl)sulfonyl)-2-fluorobenzamide

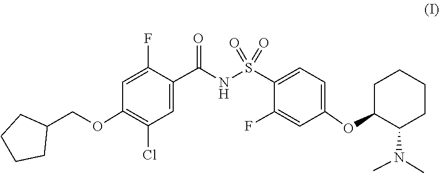 [SMILES: CN(C)[C@H]1CCCC[C@@H]1Oc1ccc(S(=O)(=O)NC(=O)c2cc(Cl)c(OCC3CCCC3)cc2F)c(F)c1]

Following the procedure described in Example 65 and making non-critical variations as required to replace 4-(cyclopentylmethoxy)-5-cyclopropyl-2-fluorobenzoic acid with 5-chloro-4-(cyclopentylmethoxy)-2-fluoro-benzoic acid, the title compound was obtained as a white solid. ¹H NMR (400 MHz, DMSO-d₆) δ 9.23 (s, 1H), 7.81-7.69 (m, 2H), 7.03-6.88 (m, 3H), 4.81-4.63 (m, 1H), 3.95 (d, J=6.8 Hz, 2H), 3.55-3.48 (m, 1H), 2.82-2.65 (m, 6H), 2.38-2.25 (m, 1H), 2.24-2.14 (m, 1H), 2.13-2.05 (m, 1H), 1.80-1.29 (m, 14H). LCMS (ESI) m/z: 571.2 [M+H]⁺.

### Example 69: (R)-5-chloro-4-(cyclopentylmethoxy)-2-fluoro-N-((4-(pyrrolidin-3-yloxy)-phenyl)sulfonyl)benzamide

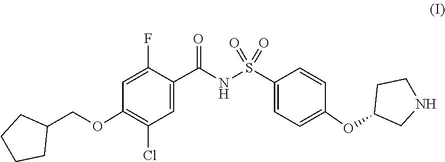 [SMILES: O=C(NS(=O)(=O)c1ccc(O[C@@H]2CCNC2)cc1)c1cc(Cl)c(OCC2CCCC2)cc1F]

### Step 1: tert-butyl 3-(4-sulfamoylphenoxy)pyrrolidine-1-carboxylate

To a stirred solution of tert-butyl 3-hydroxypyrrolidine-1-carboxylate (1.6 g, 8.56 mmol) in DMF (20 mL) was added NaH (345 mg, 8.62 mmol, 60% in mineral oil) at 0° C. After 10 min, 4-fluorobenzenesulfonamide (500 mg, 2.85 mmol) was added at 0° C. and stirred for 16 h at 80° C. After cooling to room temperature, the reaction was quenched with saturated aqueous NH₄Cl (40 mL), and extracted with EtOAc (60 mL×3). The combined organic layers were washed with brine (50 mL), dried over anhydrous Na₂SO₄, filtered and concentrated in vacuo. The crude residue was purified by silica gel chromatography (solvent gradient: 0-40% EtOAc in petroleum ether) to afford the title compound (210 mg, 22%) as a white solid. LCMS (ESI) m/z: 365.0 [M+Na]⁺.

### Step 2: tert-butyl 3-(4-(N-(5-chloro-4-(cyclopentylmethoxy)-2-fluorobenzoyl)-sulfamoyl)phenoxy)pyrrolidine-1-carboxylate

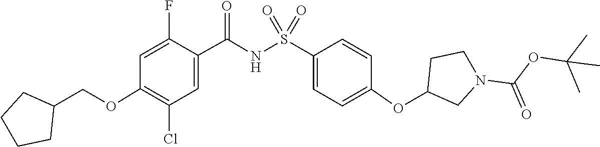 [SMILES: CC(C)(C)OC(=O)N1CCC(Oc2ccc(S(=O)(=O)NC(=O)c3cc(Cl)c(OCC4CCCC4)cc3F)cc2)C1]

Following the procedure described in Example 59 and replace 4-(((1S,2S)-2-(dimethylamino)cyclohexyl)amino)-2-fluorobenzenesulfonamide with tert-butyl 3-(4-sulfamoylphenoxy)pyrrolidine-1-carboxylate, the title compound was obtained as yellow oil. LCMS (ESI) m/z: 497.2 [M-100+H]⁺.

### Step 3: (R)-tert-butyl 3-(4-(N-(5-chloro-4-(cyclopentylmethoxy)-2-fluorobenzoyl)-sulfamoyl)phenoxy)pyrrolidine-1-carboxylate and (S)-tert-butyl 3-(4-(N-(5-chloro-4-(cyclopentylmethoxy)-2-fluorobenzoyl)sulfamoyl)-phenoxy)pyrrolidine-1-carboxylate

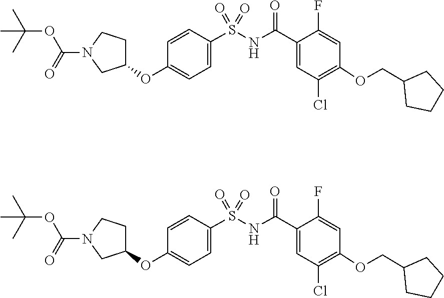 [SMILES: CC(C)(C)OC(=O)N1CC[C@H](Oc2ccc(S(=O)(=O)NC(=O)c3cc(Cl)c(OCC4CCCC4)cc3F)cc2)C1 ; CC(C)(C)OC(=O)N1CC[C@@H](Oc2ccc(S(=O)(=O)NC(=O)c3cc(Cl)c(OCC4CCCC4)cc3F)cc2)C1]

tert-butyl 3-(4-(N-(5-chloro-4-(cyclopentylmethoxy)-2-fluorobenzoyl)sulfamoyl) phenoxy)pyrrolidine-1-carboxylate (270 mg, 0.45 mmol) was separated by using chiral SFC (Chiralpak AD (250 mm*30 mm, 10 um), Supercritical CO₂/EtOH+0.1% NH₃·H₂O=50/50; 80 mL/min) to afford (R)-tert-butyl 3-(4-(N-(5-chloro-4-(cyclopentylmethoxy)-2-fluorobenzoyl)sulfamoyl)phenoxy)pyrrolidine-1-carboxylate (100 mg, first peak) and (S)-tert-butyl 3-(4-(N-(5-chloro-4-(cyclopentylmethoxy)-2-fluorobenzoyl)sulfamoyl)phenoxy)pyrrolidine-1-carboxylate (100 mg, second peak). Absolute configuration was arbitrarily assigned to each enantiomer. LCMS (ESI) m/z: 619.1 [M+Na]⁺.

### Step 4: (R)-5-chloro-4-(cyclopentylmethoxy)-2-fluoro-N-((4-(pyrrolidin-3-yloxy)phenyl)sulfonyl)benzamide

To a solution of (R)-tert-butyl 3-(4-(N-(5-chloro-4-(cyclopentylmethoxy)-2-fluorobenzoyl)sulfamoyl)phenoxy)pyrrolidine-1-carboxylate (70 mg, 0.12 mmol) in DCM (1 mL) was added trifluoroacetic acid (0.07 mL, 1 mmol) at room temperature. The mixture was stirred at room temperature for 1 h. The mixture was concentrated and purified by reverse phase chromatography (acetonitrile 25-55%/(0.05% NH₃·H₂O+10 mM NH₄HCO₃) in water) to afford the title compound (29 mg, 49%) as a white solid. ¹H NMR (400 MHz, DMSO-d₆) δ 7.80-7.70 (m, 3H), 6.98-6.85 (m, 3H), 5.12-4.99 (m, 1H), 3.93 (d, J=6.8 Hz, 2H), 3.26-3.20 (m, 2H), 3.19-3.11 (m, 2H), 2.35-2.25 (m, 1H), 2.23-2.10 (m, 1H), 2.07-1.95 (m, 1H), 1.81-1.70 (m, 2H), 1.64-1.48 (m, 4H), 1.40-1.25 (m, 2H). LCMS (ESI) m/z: 497.0 [M+H]⁺.

### Example 70: N-((4-(benzyloxy)-2-fluorophenyl)sulfonyl)-4-(cyclopentylmethoxy)-5-cyclopropyl-2-fluorobenzamide

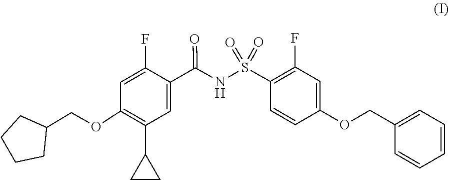 [SMILES: O=C(NS(=O)(=O)c1ccc(OCc2ccccc2)cc1F)c1cc(C2CC2)c(OCC2CCCC2)cc1F]

### Step 1: benzyl(4-(benzyloxy)-2-fluorophenyl)sulfane

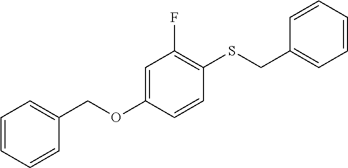 [SMILES: Fc1cc(OCc2ccccc2)ccc1SCc1ccccc1]

To a solution of 4-benzyloxy-1-bromo-2-fluoro-benzene (830 mg, 2.95 mmol), tris-(dibenzylideneacetone)dipalladium (270 mg, 0.3 mmol), 4,5-bis(diphenylphosphino)-9,9-dimethylxanthene (342 mg, 0.59 mmol) and DIPEA (1.03 mL, 5.9 mmol) in 1,4-dioxane (11 mL) under nitrogen atmosphere. Phenylmethanethiol (0.52 mL, 4.43 mmol) was added and the reaction was stirred at 120° C. under nitrogen atmosphere for 16 h. After cooling to room temperature, the reaction was diluted with water (30 mL) and extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-10% EtOAc in petroleum ether) to afford the title compound (510 mg, 53%) as colorless oil. ¹H NMR (400 MHz, CDCl₃) δ 7.59-7.51 (m, 4H), 7.50-7.45 (m, 1H), 7.41-7.29 (m, 6H), 6.90-6.80 (m, 1H), 6.79-6.69 (m, 1H), 5.16 (s, 2H), 4.10 (s, 2H).

### Step 2: 4-(benzyloxy)-2-fluorobenzenesulfonamide

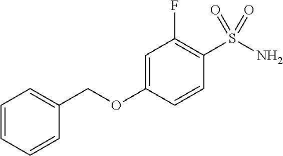 [SMILES: NS(=O)(=O)c1ccc(OCc2ccccc2)cc1F]

To a solution of benzyl(4-(benzyloxy)-2-fluorophenyl)sulfane (500 mg, 1.54 mmol) in acetonitrile (4 mL), acetic acid (3 mL) and water (3 mL) was added 1,3-dichloro-5,5-dimethylimidazolidine-2,4-dione (911 mg, 4.62 mmol) slowly at 0° C. After stirring at 0° C. for 1 h, NH₃·H₂O (2.7 mL, 36.8 mmol) was added at 0° C. The reaction was stirred at 25° C. for 2 h. The reaction was diluted with water (30 mL) and extracted with EtOAc (30 mL×3). The combined organic layers were washed with brine (30 mL), dried over anhydrous Na₂SO₄, filtered and concentrated in vacuo to afford the title compound (150 mg, crude) as colorless oil that required no further purification. ¹H NMR (400 MHz, CDCl₃) δ 7.89-7.72 (m, 1H), 7.39-7.36 (m, 6H), 6.87-6.73 (m, 1H), 5.15 (s, 2H).

### Step 3: N-((4-(benzyloxy)-2-fluorophenyl)sulfonyl)-4-(cyclopentylmethoxy)-5-cyclopropyl-2-fluorobenzamide

Following the procedure described in Example 63 and replace 4-(((1S,2S)-2-(dimethylamino)cyclohexyl)amino)-2-fluorobenzenesulfonamide with 4-(benzyloxy)-2-fluorobenzenesulfonamide, the title compound was obtained as a white solid. ¹H NMR (400 MHz, DMSO-d₆) δ 12.56 (s, 1H), 7.87 (t, J=8.4 Hz, 1H), 7.50-7.33 (m, 5H), 7.17 (d, J=12.4 Hz, 1H), 7.07 (br d, J=8.0 Hz, 2H), 6.91 (d, J=12.8 Hz, 1H), 5.22 (s, 2H), 3.95 (d, J=6.8 Hz, 2H), 2.40-2.25 (m, 1H), 2.04-1.92 (m, 1H), 1.85-1.70 (m, 2H), 1.69-1.45 (m, 4H), 1.41-1.29 (m, 2H), 0.92-0.82 (m, 2H), 0.73-0.59 (m, 2H). LCMS (ESI) m/z: 542.1[M+H]⁺.

### Example 72: 5-chloro-4-(cyclopentylmethoxy)-2-fluoro-N-((1-(5-fluoro-2-methoxy-phenyl)isoquinolin-6-yl)sulfonyl)benzamide

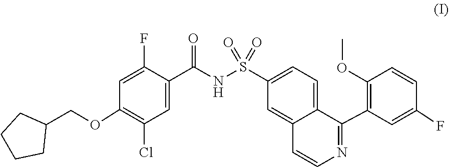 [SMILES: COc1ccc(F)cc1-c1nccc2cc(S(=O)(=O)NC(=O)c3cc(Cl)c(OCC4CCCC4)cc3F)ccc12]

### Step 1: 6-(benzylthio)-1-(5-fluoro-2-methoxyphenyl)isoquinoline

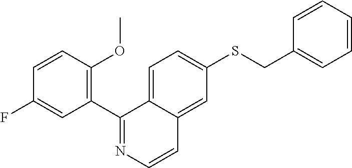 [SMILES: COc1ccc(F)cc1-c1nccc2cc(SCc3ccccc3)ccc12]

To a solution of (5-fluoro-2-methoxyphenyl)boronic acid (892 mg, 5.25 mmol) and 6-(benzylthio)-1-chloroisoquinoline (1.0 g, 3.5 mmol) in 1,4-dioxane (20 mL) and water (2 mL) was added K₃PO₄ (2.2 g, 10.5 mmol) and bis(di-tert-butyl(4-dimethylaminophenyl)phosphine)-dichloropalladium(II) (248 mg, 0.35 mmol) at room temperature under nitrogen atmosphere. Then the mixture was stirred at 100° C. for 16 h. After cooling to room temperature, the reaction was diluted with water (50 mL) and extracted with EtOAc (50 mL×3). The combined organic layers were washed with brine (100 mL), dried over anhydrous Na₂SO₄, filtered and concentrated in vacuo. The crude residue was purified by silica gel chromatography (solvent gradient: 0-30% EtOAc in petroleum ether) to afford the title compound (1.1 g, 84%) as yellow oil. LCMS (ESI) m/z: 376.2 [M+H]⁺.

### Step 2: 5-chloro-4-(cyclopentylmethoxy)-2-fluoro-N-((1-(5-fluoro-2-methoxyphenyl)isoquinolin-6-yl)sulfonyl)benzamide

Following the procedure described in Example 71 and making non-critical variations as required to replace trans-2-(4-(benzylthio)-3-fluorophenyl)-N,N-dimethylcyclohexanamine with 6-(benzylthio)-1-(5-fluoro-2-methoxyphenyl)isoquinoline, the title compound was obtained as a white solid. ¹H NMR (400 MHz, DMSO-d₆) δ 8.79-8.70 (m, 2H), 8.21 (d, J=5.6 Hz, 1H), 8.03-7.99 (m, 1H), 7.82 (d, J=8.8 Hz, 1H), 7.71 (d, J=8.0 Hz, 1H), 7.43-7.35 (m, 1H), 7.27-7.22 (m, 2H), 7.20 (d, J=12.8 Hz, 1H), 4.01 (d, J=6.8 Hz, 2H), 3.64 (s, 3H), 2.36-2.27 (m, 1H), 1.81-1.40 (m, 2H), 1.64-1.50 (m, 4H), 1.37-1.28 (m, 2H). LCMS (ESI) m/z: 587.2 [M+H]⁺.

### Example 74: 5-cyclopropyl-N-((4-(((1S,2S,4S)-2-(dimethylamino)-4-(3-(trifluoro-methyl)phenyl)cyclohexyl)oxy)-2-fluorophenyl)sulfonyl)-2-fluoro-4-methoxybenzamide

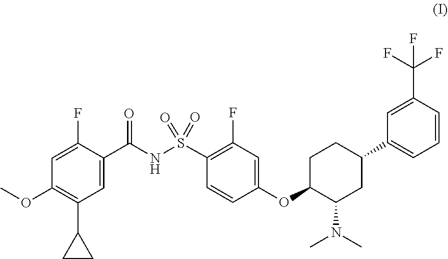 [SMILES: COc1cc(F)c(C(=O)NS(=O)(=O)c2ccc(O[C@H]3CC[C@H](c4cccc(C(F)(F)F)c4)C[C@@H]3N(C)C)cc2F)cc1C1CC1]

### Step 1: tert-butyl (2,4-difluorophenyl)sulfonyl(2,4-dimethoxybenzyl)carbamate

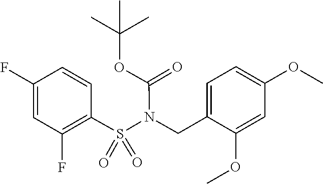 [SMILES: COc1ccc(CN(C(=O)OC(C)(C)C)S(=O)(=O)c2ccc(F)cc2F)c(OC)c1]

To a stirred solution of 2,4-dimethoxybenzylamine (7.87 g, 47.04 mmol) and pyridine (19 mL, 235.18 mmol) in DCM (220 mL) was added 2,4-difluorobenzenesulfonylchloride (10.0 g, 47.04 mmol) at 0° C. The reaction was stirred at room temperature for 1 h. Boc₂O (50.85 g, 233 mmol) and DMAP (5.69 g, 46.6 mmol) were added and the mixture was stirred at 40° C. for 16 h. After cooling to room temperature, the reaction was diluted with water (100 mL) and extracted with DCM (150 mL×3). The combined organic layers were washed with brine (100 mL), dried over anhydrous Na₂SO₄, filtered and concentrated in vacuo. The crude residue was purified by silica gel chromatography (solvent gradient: 0-15% EtOAc in petroleum ether) to afford the title compound (15 g, 73%) as a yellow solid. LCMS (ESI) m/z: 466.1 [M+Na]⁺.

### Step 2: tert-butyl 2,4-dimethoxybenzyl((4-(((1S,2S,4S)-2-(dimethylamino)-4-(3-(trifluoromethyl)phenyl)cyclohexyl)oxy)-2-fluorophenyl)sulfonyl)carbamate

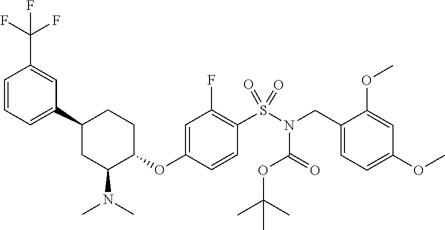 [SMILES: COc1ccc(CN(C(=O)OC(C)(C)C)S(=O)(=O)c2ccc(O[C@H]3CC[C@H](c4cccc(C(F)(F)F)c4)C[C@@H]3N(C)C)cc2F)c(OC)c1]

To a solution of NaH (100 mg, 2.51 mmol, 60% in mineral oil) in DMF (18 mL) was added (1S,2S, 4S)-2-(dimethylamino)-4-[3-(trifluoromethyl)phenyl]cyclo hexanol (0.6 g, 2.09 mmol) at 0° C. under nitrogen atmosphere. After stirring at 0° C. for 0.5 h, tert-butyl N-(2,4-difluorophenyl)sulfonyl-N-[(2,4-dimethoxyphenyl)methyl]carbamate (1.08 g, 2.09 mmol) was added dropwise at 0° C. The reaction was stirred at room temperature for 16 h. The reaction was quenched with saturated aqueous NH₄Cl (50 mL), extracted with EtOAc (50 mL×3). The combined organic layers were washed with brine (50 mL×5), dried over anhydrous Na₂SO₄, filtered and concentrated in vacuo. The crude residue was purified by silica gel chromatography (solvent gradient: 0-30% EtOAc in petroleum ether then 5% MeOH in DCM) to afford the title compound (0.60 g, 40%) as colorless oil. LCMS (ESI) m/z: 711.1 [M+H]⁺.

### Step 3: 4-(((1S,2S,4S)-2-(dimethylamino)-4-(3-(trifluoromethyl)phenyl)cyclohexyl)oxy)-2-fluorobenzenesulfonamide

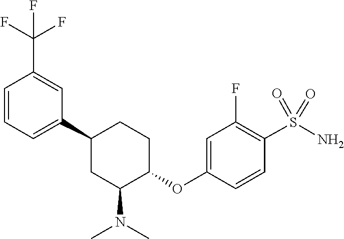 [SMILES: CN(C)[C@H]1C[C@@H](c2cccc(C(F)(F)F)c2)CC[C@@H]1Oc1ccc(S(N)(=O)=O)c(F)c1]

To a stirred solution of tert-butyl 2,4-dimethoxybenzyl((4-(((1S,2S,4S)-2-(dimethylamino)-4-(3-(trifluoromethyl)phenyl)cyclohexyl)oxy)-2-fluorophenyl)sulfonyl) carbamate (0.6 g, 0.84 mmol) in DCM (30 mL) was added TFA (96 mg, 0.84 mmol) and triethylsilane (1.41 mL, 8.81 mmol) at room temperature. Then the reaction was stirred at room temperature for 1 h. The mixture was concentrated in vacuo. The crude residue was purified by silica gel chromatography (solvent gradient: 0-10% MeOH in DCM) to afford the title compound (0.32 g, 82%) as yellow oil. LCMS (ESI) m/z: 461.3 [M+H]⁺.

### Step 4: 5-cyclopropyl-N-((4-(((1S,2S,4S)-2-(dimethylamino)-4-(3-(trifluoromethyl)-phenyl)cyclohexyl)oxy)-2-fluorophenyl)sulfonyl)-2-fluoro-4-methoxybenzamide

Following the procedure described in Example 59 and making non-critical variations as required to replace 4-(((1S,2S)-2-(dimethylamino)cyclohexyl)amino)-2-fluorobenzenesulfonamide and 5-chloro-4-(cyclopentylmethoxy)-2-fluorobenzoic acid with 4-(((1S,2S,4S)-2-(dimethylamino)-4-(3-(trifluoromethyl)phenyl)cyclohexyl)oxy)-2-fluorobenzenesulfonamide and 5-cyclopropyl-2-fluoro-4-methoxybenzoic acid, the title compound was obtained as a white solid. ¹H NMR (400 MHz, DMSO-d₆) δ 9.31 (s, 1H), 7.76 (t, J=8.8 Hz, 1H), 7.71-6.68 (m, 1H), 7.65-7.54 (m, 3H), 7.23 (d, J=8.4 Hz, 1H), 6.97-6.86 (m, 2H), 6.69 (d, J=12.8 Hz, 1H), 4.91-4.79 (m, 1H), 3.81 (s, 3H), 3.77-3.58 (m, 1H), 2.96-2.89 (m, 1H), 2.85-2.60 (m, 6H), 2.36-2.28 (m, 1H), 2.25-2.15 (m, 1H), 2.01-1.94 (m, 1H), 1.90-1.75 (m, 3H), 1.57-1.45 (m, 1H), 0.90-0.81 (m, 2H), 0.55-0.47 (m, 2H). LCMS (ESI) m/z: 653.3 [M+H]⁺.

### Example 75: 5-cyclopropyl-N-((4-(((1S,2S,4S)-2-(dimethylamino)-4-(3-(trifluoromethyl)-phenyl)cyclohexyl)oxy)-2-fluorophenyl)sulfonyl)-2-fluorobenzamide

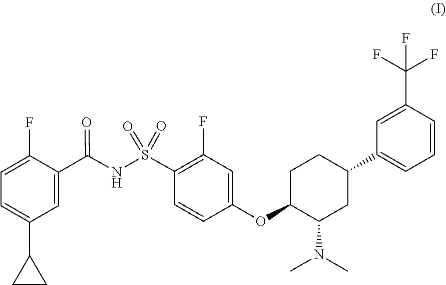 [SMILES: CN(C)[C@H]1C[C@@H](c2cccc(C(F)(F)F)c2)CC[C@@H]1Oc1ccc(S(=O)(=O)NC(=O)c2cc(C3CC3)ccc2F)c(F)c1]

### Step 1: 5-cyclopropyl-2-fluorobenzoic acid

To a solution of methyl 5-cyclopropyl-2-fluorobenzoate (580 mg, 2.99 mmol) in water (7.5 mL) and THF (7.5 mL) was added LiOH (716 mg, 30 mmol) at room temperature and the reaction was stirred at room temperature for 16 h. The reaction was quenched with aqueous HCl (1 M) to pH=2, and extracted with EtOAc (20 mL×3). The combined organic layers were washed with brine (30 mL), dried over anhydrous Na₂SO₄, filtered and concentrated in vacuo to afford the title compound (420 mg, 71%) as a white solid. LCMS (ESI) m/z: 181.1 [M+H]⁺.

### Step 2: 5-cyclopropyl-N-((4-(((1S,2S,4S)-2-(dimethylamino)-4-(3-(trifluoromethyl)phenyl)cyclohexyl)oxy)-2-fluorophenyl)sulfonyl)-2-fluorobenzamide

Following the procedure described in Example 74 and making non-critical variations as required to replace 5-cyclopropyl-2-fluoro-4-methoxybenzoic acid with 5-cyclopropyl-2-fluorobenzoic acid, the title compound was obtained as a white solid. ¹H NMR (400 MHz, DMSO-d₆) δ 7.77-7.72 (m, 1H), 7.69-7.55 (m, 4H), 7.39-7.30 (m, 1H), 7.06-6.97 (m, 1H), 6.96-6.86 (m, 3H), 4.85-4.75 (m, 1H), 3.83-3.76 (m, 1H), 2.95-2.87 (m, 1H), 2.63 (s, 6H), 2.33-2.25 (m, 1H), 2.20-2.05 (m, 1H), 1.93-1.61 (m, 3H), 1.57-1.42 (m, 1H), 1.23-1.16 (m, 1H), 0.96-0.86 (m, 2H), 0.62-0.53 (m, 2H). LCMS (ESI) m/z: 623.3 [M+H]⁺.

### Example 76: 5-chloro-4-(cyclopentylmethoxy)-N-((4-(3-(dimethylamino)-3-methylpiperidin-1-yl)-2,6-difluorophenyl)sulfonyl)-2-fluorobenzamide

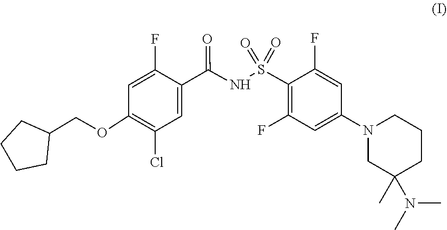 [SMILES: CN(C)C1(C)CCCN(c2cc(F)c(S(=O)(=O)NC(=O)c3cc(Cl)c(OCC4CCCC4)cc3F)c(F)c2)C1]

### Step 1: tert-butyl 3-(dimethylamino)-3-methylpiperidine-1-carboxylate

To a mixture of tert-butyl 3-amino-3-methylpiperidine-1-carboxylate (0.4 g, 1.87 mmol) in MeCN (20 mL) was added formaldehyde (16.22 mL, 215.78 mmol, 2 M in THF) and NaBH₃CN (0.59 g, 9.33 mmol). The mixture was stirred at room temperature for 16 h. The reaction was quenched with saturated aqueous NaHCO₃ solution (15 mL) to pH>7, and extracted with EtOAc (30 mL×2). The combined organic layers were washed with brine (50 mL), dried over anhydrous Na₂SO₄, filtered and concentrated in vacuo to afford the title compound (425 mg, 94%) as yellow oil that required no further purification. ¹H NMR (400 MHz, CDCl₃) δ 3.66-3.35 (m, 2H), 3.26-3.02 (m, 2H), 2.25 (s, 6H), 1.72-1.47 (m, 4H), 1.46 (s, 9H), 0.88 (s, 3H).

### Step 2: N,N, 3-trimethylpiperidin-3-amine

To a stirred solution of tert-butyl 3-(dimethylamino)-3-methyl-piperidine-1-carboxylate (0.4 g, 1.65 mmol) in DCM (8 mL) was added TFA (3 mL). The reaction was stirred at room temperature for 16 h. The reaction was concentrated in vacuo to remove most solvent and diluted with water (20 mL), neutralized with 10% aqueous NaOH solution (pH>7) and extracted with DCM (20 mL×3). The combined organic layers were washed with brine (50 mL), dried over anhydrous Na₂SO₄, filtered and concentrated in vacuo to afford the title compound (220 mg, crude) as yellow oil that required no further purification.

### Step 3: tert-butyl 2,4-dimethoxybenzyl((4-(3-(dimethylamino)-3-methylpiperidin-1-yl)-2,6-difluorophenyl)sulfonyl)carbamate

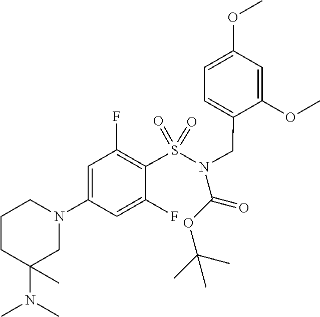 [SMILES: COc1ccc(CN(C(=O)OC(C)(C)C)S(=O)(=O)c2c(F)cc(N3CCCC(C)(N(C)C)C3)cc2F)c(OC)c1]

Following the procedure described in Example 62 and making non-critical variations as required to replace N,N-dimethylpiperidin-3-amine with N,N, 3-trimethylpiperidin-3-amine and replace 2,4,6-trifluorobenzenesulfonamide with tert-butyl 2,4-dimethoxybenzyl((2,4,6-trifluorophenyl)sulfonyl)carbamate, the title compound was obtained as colorless oil. LCMS (ESI) m/z: 584.3 [M+H]⁺.

### Step 4: 5-chloro-4-(cyclopentylmethoxy)-N-((4-(3-(dimethylamino)-3-methylpiperidin-1-yl)-2,6-difluorophenyl)sulfonyl)-2-fluorobenzamide

Following the procedure described in Example 74 and making non-critical variations as required to replace tert-butyl 2,4-dimethoxybenzyl((4-(((1S,2S,4S)-2-(dimethylamino)-4-(3-(trifluoromethyl)phenyl)cyclohexyl)oxy)-2-fluorophenyl)sulfonyl)carbamate with tert-butyl 2,4-dimethoxybenzyl((4-(3-(dimethylamino)-3-methylpiperidin-1-yl)-2,6-difluorophenyl)sulfonyl)carbamate and replace 5-cyclopropyl-2-fluoro-4-methoxybenzoic acid with 5-chloro-4-(cyclopentylmethoxy)-2-fluorobenzoic acid, the title compound was obtained as a white solid. ¹H NMR (400 MHz, DMSO-d₆) δ 7.74 (d, J=8.0 Hz, 1H), 6.94 (d, J=12.4 Hz, 1H), 6.60 (d, J=12.4 Hz, 2H), 3.95 (d, J=6.8 Hz, 2H), 3.47-3.35 (m, 2H), 3.30-3.15 (m, 2H), 2.57 (s, 6H), 2.36-2.29 (m, 1H), 1.80-1.69 (m, 5H), 1.63-1.51 (m, 5H), 1.39-1.32 (m, 2H), 1.12 (s, 3H). LCMS (ESI) m/z: 588.2 [M+H]⁺.

### Example 78: 5-chloro-4-(cyclopentylmethoxy)-2-fluoro-N-(phenylsulfonyl)benzamide

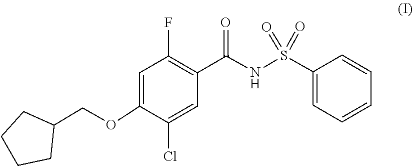 [SMILES: O=C(NS(=O)(=O)c1ccccc1)c1cc(Cl)c(OCC2CCCC2)cc1F]

Following the procedure described in Example 59 and making non-critical variations as required to replace 4-(((1S,2S)-2-(dimethylamino)cyclohexyl)amino)-2-fluorobenzene-sulfonamide with benzenesulfonamide, the title compound was obtained as a white solid. ¹H NMR (400 MHz, DMSO-d₆) δ 12.48 (s, 1H), 7.98 (d, J=7.2 Hz, 2H), 7.77-7.70 (m, 1H) 7.70-7.63 (m, 3H), 7.21 (d, J=12.8 Hz, 1H), 4.01 (d, J=6.8 Hz, 2H), 2.37-2.28 (m, 1H), 1.81-1.70 (m, 2H), 1.65-1.47 (m, 4H), 1.39-1.28 (m, 2H). LCMS (ESI) m/z: 412.2 [M+H]⁺.

### Example 79: 5-chloro-4-(cyclopentylmethoxy)-N-((2,6-difluorophenyl)sulfonyl)-2-fluorobenzamide

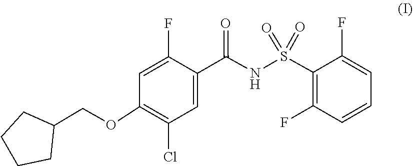 [SMILES: O=C(NS(=O)(=O)c1c(F)cccc1F)c1cc(Cl)c(OCC2CCCC2)cc1F]

Following the procedure described in Example 59 and replace 4-(((1S,2S)-2-(dimethylamino)cyclohexyl)amino)-2-fluorobenzenesulfonamide with 2,6-difluorobenzenesulfonamide, the title compound was obtained as a white solid. ¹H NMR (400 MHz, DMSO-d₆) δ 7.83-7.75 (m, 1H), 7.73 (d, J=7.6 Hz, 1H), 7.33 (t, J=9.2 Hz, 2H), 7.22 (d, J=12.4 Hz, 1H), 4.02 (d, J=6.8 Hz, 2H), 2.38-2.26 (m, 1H), 1.83-1.70 (m, 2H), 1.68-1.48 (m, 4H), 1.40-1.29 (m, 2H). LCMS (ESI) m/z: 447.9 [M+H]⁺.

### Example 80: 5-chloro-N-((5-chloro-2-fluorophenyl)sulfonyl)-4-(cyclopentylmethoxy)-2-fluorobenzamide

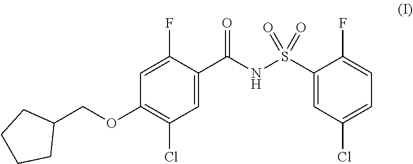 [SMILES: O=C(NS(=O)(=O)c1cc(Cl)ccc1F)c1cc(Cl)c(OCC2CCCC2)cc1F]

Following the procedure described in Example 1 and making non-critical variations as required to replace 5-chloro-2,4-difluorobenzene-1-sulfonyl chloride and 4-(cyclopentylmethoxy)-5-cyclopropyl-2-fluorobenzoic acid with 5-chloro-2-fluorobenzene-1-sulfonyl chloride and 5-chloro-4-(cyclopentylmethoxy)-2-fluorobenzoic acid, the title compound was obtained as a white solid. ¹H NMR (400 MHz, DMSO-d₆) δ 7.93-7.87 (m, 1H), 7.86-7.81 (m, 1H), 7.73 (d, J=7.6 Hz, 1H), 7.58-7.49 (m, 1H), 7.19 (d, J=12.4 Hz, 1H), 4.01 (d, J=7.2 Hz, 2H), 2.38-2.28 (m, 1H), 1.80-1.71 (m, 2H), 1.65-1.51 (m, 4H), 1.38-1.31 (m, 2H). LCMS (ESI) m/z: 464.1 [M+H]⁺.

### Example 81: 5-chloro-4-(cyclopentylmethoxy)-2-fluoro-N-((4-methoxyphenyl)sulfonyl)-benzamide

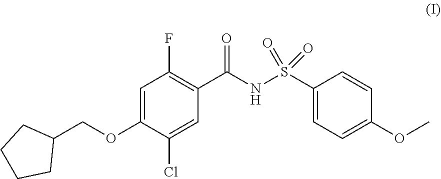 [SMILES: COc1ccc(S(=O)(=O)NC(=O)c2cc(Cl)c(OCC3CCCC3)cc2F)cc1]

Following the procedure described in Example 59 and replace 4-(((1S,2S)-2-(dimethylamino)cyclohexyl)amino)-2-fluorobenzenesulfonamide with 4-methoxy-benzenesulfonamide, the title compound was obtained as a white solid. ¹H NMR (400 MHz, DMSO-d₆) δ 12.32 (s, 1H), 7.92 (d, J=8.8 Hz, 2H), 7.66 (d, J=7.6 Hz, 1H), 7.21 (d, J=12.4 Hz, 1H), 7.16 (d, J=8.8 Hz, 2H), 4.01 (d, J=6.8 Hz, 2H), 3.86 (s, 3H), 2.37-2.25 (m, 1H), 1.82-1.70 (m, 2H), 1.66-1.47 (m, 4H), 1.39-1.27 (m, 2H), LCMS (ESI) m/z: 442.0 [M+H]⁺.

### Example 82: 5-chloro-N-(cyclohexylsulfonyl)-4-(cyclopentylmethoxy)-2-fluorobenzamide

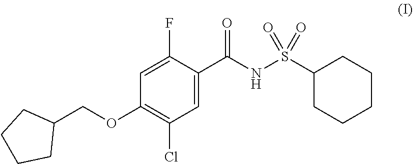 [SMILES: O=C(NS(=O)(=O)C1CCCCC1)c1cc(Cl)c(OCC2CCCC2)cc1F]

Following the procedure described in Example 80 and making non-critical variations as required to replace 5-chloro-2-fluorobenzene-1-sulfonyl chloride with cyclohexanesulfonyl chloride, the title compound was obtained as a white solid. ¹H NMR (400 MHz, DMSO-d₆) δ 11.94 (s, 1H), 7.74 (d, J=7.6 Hz, 1H), 7.23 (d, J=12.4 Hz, 1H), 4.03 (d, J=6.8 Hz, 2H), 3.55-3.45 (m, 1H), 2.39-2.28 (m, 1H), 2.09-2.01 (m, 2H), 1.88-1.71 (m, 4H), 1.68-1.59 (m, 3H), 1.58-1.51 (m, 2H), 1.50-1.41 (m, 2H), 1.40-1.23 (m, 4H), 1.22-1.09 (m, 1H). LCMS (ESI) m/z: 418.1 [M+H]⁺.

### Example 83: N-((4-(benzyloxy)phenyl)sulfonyl)-4-(cyclopentylmethoxy)-5-cyclopropyl-2-fluorobenzamide

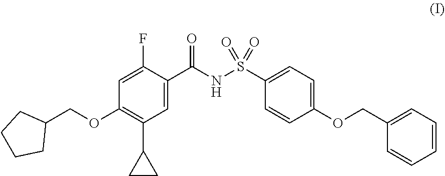 [SMILES: O=C(NS(=O)(=O)c1ccc(OCc2ccccc2)cc1)c1cc(C2CC2)c(OCC2CCCC2)cc1F]

Following the procedure described in Example 69 and making non-critical variations as required to replace tert-butyl 3-hydroxypyrrolidine-1-carboxylate and 5-chloro-4-(cyclopentylmethoxy)-2-fluorobenzoic acid with phenylmethanol and 4-(cyclopentylmethoxy)-5-cyclopropyl-2-fluorobenzoic acid, the title compound was obtained as a white solid. ¹H NMR (400 MHz, DMSO-d₆) δ 12.09 (s, 1H), 7.91 (d, J=8.8 Hz, 2H), 7.49-7.33 (m, 5H), 7.23 (d, J=8.8 Hz, 2H), 7.04 (d, J=8.4 Hz, 1H), 6.91 (d, J=13.2 Hz, 1H), 5.21 (s, 2H), 3.94 (d, J=6.8 Hz, 2H), 2.37-2.25 (m, 1H), 2.01-1.93 (m, 1H), 1.85-1.70 (m, 2H), 1.65-1.48 (m, 4H), 1.41-1.30 (m, 2H), 0.89-0.81 (m, 2H), 0.68-0.60 (m, 2H). LCMS (ESI) m/z: 524.2 [M+H]⁺.

### Example 84: N-((4-(benzylamino)phenyl)sulfonyl)-5-chloro-4-(cyclopentylmethoxy)-2-fluorobenzamide

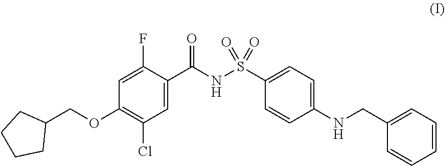 [SMILES: O=C(NS(=O)(=O)c1ccc(NCc2ccccc2)cc1)c1cc(Cl)c(OCC2CCCC2)cc1F]

Following the procedure described in Example 59 and making non-critical variations as required to replace 4-(((1S,2S)-2-(dimethylamino) cyclohexyl) amino)-2-fluorobenzene-sulfonamide with 4-(benzylamino)benzenesulfonamide (Reference: Bioorg. Med. Chem. Lett., 2014, 24, 1776), the title compound was obtained as a white solid. ¹H NMR (400 MHz, DMSO-d₆) δ 12.02 (s, 1H), 7.67-7.59 (m, 3H), 7.36-7.33 (m, 4H), 7.27-7.22 (m, 1H), 7.19 (d, J=11.6 Hz, 1H), 6.68 (d, J=8.8 Hz, 2H), 4.35 (d, J=5.6 Hz, 2H), 4.00 (d, J=6.8 Hz, 2H), 2.37-2.27 (m, 1H), 1.83-1.70 (m, 2H), 1.65-1.49 (m, 4H), 1.48-1.27 (m, 2H). LCMS (ESI) m/z: 517.1 [M+H]⁺.

### Example 85: 5-chloro-4-(cyclopentylmethoxy)-2-fluoro-N-((4-(pyridin-4-ylmethoxy)-phenyl)sulfonyl)benzamide

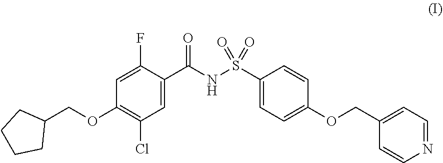 [SMILES: O=C(NS(=O)(=O)c1ccc(OCc2ccncc2)cc1)c1cc(Cl)c(OCC2CCCC2)cc1F]

Following the procedure described in Example 83 and making non-critical variations as required to replace phenylmethanol and 4-(cyclopentylmethoxy)-5-cyclopropyl-2-fluorobenzoic acid with pyridin-4-yl methanol and 5-chloro-4-(cyclopentylmethoxy)-2-fluorobenzoic acid, the title compound was obtained as a white solid. ¹H NMR (400 MHz, DMSO-d₆) δ 8.60 (d, J=5.6 Hz, 2H), 7.92 (d, J=9.2 Hz, 2H), 7.67 (d, J=7.2 Hz, 1H), 7.46 (d, J=5.6 Hz, 2H), 7.23 (d, J=9.2 Hz, 2H), 7.19 (d, J=12.4 Hz, 1H), 5.30 (s, 2H), 4.00 (d, J=6.8 Hz, 2H), 2.37-2.25 (m, 1H), 1.80-1.71 (m, 2H), 1.65-1.46 (m, 4H), 1.37-1.25 (m, 2H). LCMS (ESI) m/z: 519.2 [M+H]⁺.

### Example 86 and Example 87: trans-5-chloro-4-(cyclopentylmethoxy)-2-fluoro-N-((4-methoxycyclohexyl)sulfonyl)benzamide, and cis-5-chloro-4-(cyclopentylmethoxy)-2-fluoro-N-((4-methoxycyclohexyl)sulfonyl)benzamide

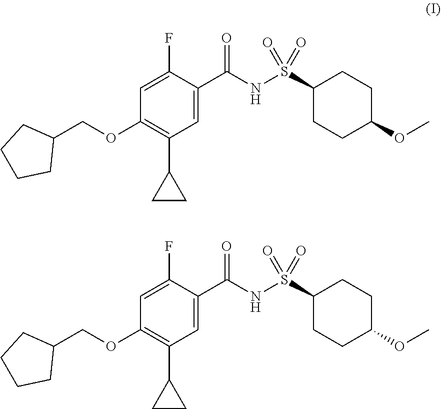 [SMILES: CO[C@H]1CC[C@@H](S(=O)(=O)NC(=O)c2cc(C3CC3)c(OCC3CCCC3)cc2F)CC1 ; CO[C@H]1CC[C@H](S(=O)(=O)NC(=O)c2cc(C3CC3)c(OCC3CCCC3)cc2F)CC1]

(These compounds may also be referred to as 5-chloro-4-(cyclopentylmethoxy)-2-fluoro-N-(((1r, 4r)-4-methoxycyclohexyl)sulfonyl)benzamide, and 5-chloro-4-(cyclopentylmethoxy)-2-fluoro-N-(((1s, 4s)-4-methoxycyclohexyl)sulfonyl)benzamide.

To a solution of NaH (28.21 mg, 0.71 mmol, 60% in mineral oil) in THF (4 mL) was added 5-chloro-4-(cyclopentylmethoxy)-2-fluoro-benzamide (113 mg, 0.42 mmol) at 0° C. and the reaction was stirred at 0° C. for 0.5 h. 4-methoxycyclohexanesulfonyl chloride (100 mg, 0.47 mmol) was added and the reaction was stirred at room temperature for 3 h. The reaction was then quenched with saturated aqueous NH4Cl solution (30 mL), extracted with EtOAc (30 mL×3). The combined organic layers were washed with brine (50 mL), dried over anhydrous Na₂SO₄, filtered and concentrated in vacuo. The crude residue was purified by reverse phase chromatography (acetonitrile 65-95%/0.2% HCOOH in water) to afford cis-5-chloro-4-(cyclopentylmethoxy)-2-fluoro-N-((4-methoxycyclohexyl) sulfonyl)benzamide (10 mg, first peak) and trans-5-chloro-4-(cyclopentylmethoxy)-2-fluoro-N-((4-methoxycyclohexyl)sulfonyl)-benzamide (10 mg, second peak) both as a white solid. Example 86: trans ¹H NMR (400 MHz, DMSO-d₆) δ 12.00 (s, 1H), 7.75 (d, J=7.6 Hz, 1H), 7.24 (d, J=12.4 Hz, 1H), 4.03 (d, J=6.8 Hz, 2H), 3.53-3.43 (m, 1H), 3.23 (s, 3H), 3.17-3.07 (m, 1H), 2.40-2.27 (m, 1H), 2.15-2.03 (m, 4H), 1.83-1.73 (m, 2H), 1.65-1.50 (m, 6H), 1.39-1.29 (m, 2H), 1.26-1.14 (m, 2H). LCMS (ESI) m/z: 448.2 [M+H]⁺. Example 87: cis ¹H NMR (400 MHz, CDCl₃) δ 8.45 (s, 1H), 8.07 (m, J=8.0 Hz, 1H), 6.70 (d, J=13.6 Hz, 1H), 3.96 (d, J=6.8 Hz, 2H), 3.78-3.68 (m, 1H), 3.50-3.45 (m, 1H), 3.30 (s, 3H), 2.51-2.40 (m, 1H), 2.17-2.08 (m, 2H), 2.05-1.95 (m, 2H), 1.93-1.84 (m, 2H), 1.73-1.59 (m, 6H), 1.49-1.37 (m, 4H). LCMS (ESI) m/z: 448.2 [M+H]⁺.

### Example 88: N-(5-chloro-6-(cyclopentylmethoxy)benzo[d]isoxazol-3-yl)-4-(((1S,2S)-2-(dimethylamino)cyclohexyl)oxy)-2-fluorobenzenesulfonamide

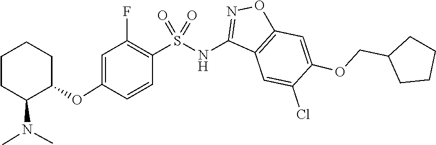 [SMILES: CN(C)[C@H]1CCCC[C@@H]1Oc1ccc(S(=O)(=O)Nc2noc3cc(OCC4CCCC4)c(Cl)cc23)c(F)c1]

### Step 1: 5-chloro-6-(cyclopentylmethoxy)benzo[d]isoxazol-3-amine

To a solution of 5-chloro-4-(cyclopentylmethoxy)-2-fluorobenzonitrile (2 g, 7.88 mmol) in DMF (40 mL) was added t-BuOK (2.65 g, 23.65 mmol) at room temperature under nitrogen atmosphere. The reaction was stirred at room temperature for 30 min. Then N-hydroxyacetamide (1.8 g, 23.65 mmol) was added and then the reaction was stirred at 50° C. for 16 h. The reaction was diluted with water (80 mL) and extracted with EtOAc (150 mL×3). The combined organic lawyers were washed with brine (100 mL), dried over anhydrous Na₂SO₄, filtered and concentrated in vacuo. The crude residue was purified by silica gel chromatography (solvent gradient: 0-20% EtOAc in petroleum ether) to afford the title compound (820 mg, 39%) as a white solid. LCMS (ESI) m/z: 267.1 [M+H]⁺.

### Step 2: 5-chloro-6-(cyclopentylmethoxy)-N-(2,4-dimethoxybenzyl)benzo[d]isoxazol-3-amine

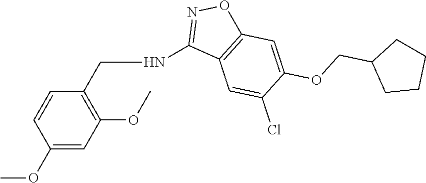 [SMILES: COc1ccc(CNc2noc3cc(OCC4CCCC4)c(Cl)cc23)c(OC)c1]

To a stirred solution of 5-chloro-6-(cyclopentylmethoxy)benzo[d]isoxazol-3-amine (0.71 g, 2.65 mmol) and 2,4-dimethoxybenzaldehyde (0.4 g, 2.41 mmol) in DCM (12 mL) was added TiCl(Oi-Pr)₃ (1.86 mL, 5.54 mmol) in one portion under nitrogen atmosphere. The solution was stirred for 10 min before the portion wise addition of NaBH(OAc)₃ (1.53 g, 7.22 mmol) at 0° C. The reaction was stirred at room temperature for 16 h. The reaction was quenched with saturated aqueous NaHCO₃ solution (50 mL), extracted with DCM (50 mL×3). The combined organic lawyers were washed with brine (50 mL), dried over anhydrous Na₂SO₄, filtered and concentrated in vacuo. The crude residue was purified by silica gel chromatography (solvent gradient: 0-25% EtOAc in petroleum ether) to afford the title compound (0.53 g, 53%) as a white solid. LCMS (ESI) m/z: 417.2 [M+H]⁺.

### Step 3: N-(5-chloro-6-(cyclopentylmethoxy)benzo[d]isoxazol-3-yl)-N-(2,4-dimethoxybenzyl)-2,4-difluorobenzenesulfonamide

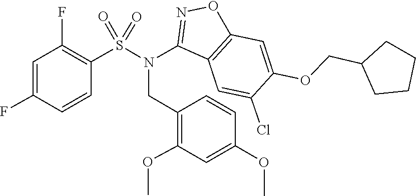 [SMILES: COc1ccc(CN(c2noc3cc(OCC4CCCC4)c(Cl)cc23)S(=O)(=O)c2ccc(F)cc2F)c(OC)c1]

To a solution of 5-chloro-6-(cyclopentylmethoxy)-N-(2,4-dimethoxybenzyl)-benzo[d]isoxazol-3-amine (170 mg, 0.52 mmol) in THF (2 mL) was added LiHMDS (0.62 mL, 0.62 mmol, 1 M) at −78° C. The reaction was stirred for 30 min at 0° C. and a solution of 2,4-difluorobenzenesulfonylchloride (0.22 g, 1.03 mmol) in THF (2 mL) was added dropwise at −78° C. After the addition was complete, the cooling bath was removed. The reaction mixture was stirred at room temperature for 3 h. The reaction was diluted with water (30 mL) and extracted with EtOAc (50 mL×3). The combined organic lawyers were washed with brine (50 mL), dried over anhydrous Na₂SO₄, filtered and concentrated in vacuo. The crude residue was purified by silica gel chromatography (solvent gradient: 0-30% EtOAc in petroleum ether) to afford the title compound (160 mg, 52%) as a white solid. ¹H NMR (400 MHz, CDCl₃) δ 7.92-7.78 (m, 1H), 7.62 (s, 1H), 7.25-7.14 (m, 1H), 7.12-6.84 (m, 3H), 6.44-6.27 (m, 2H), 5.00 (s, 2H), 3.92 (d, J=6.8 Hz, 2H), 3.75 (s, 3H), 3.58 (s, 3H), 2.56-2.35 (m, 1H), 1.97-1.84 (m, 2H), 1.74-1.55 (m, 4H), 1.49-1.37 (m, 2H).

### Step 4: N-(5-chloro-6-(cyclopentylmethoxy)benzo[d]isoxazol-3-yl)-N-(2,4-dimethoxybenzyl)-4-(((1S,2S)-2-(dimethylamino)cyclohexyl)oxy)-2-fluorobenzenesulfonamide

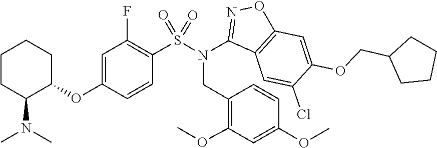 [SMILES: COc1ccc(CN(c2noc3cc(OCC4CCCC4)c(Cl)cc23)S(=O)(=O)c2ccc(O[C@H]3CCCC[C@@H]3N(C)C)cc2F)c(OC)c1]

To a stirred solution of NaH (9 mg, 0.2 mmol, 60% in mineral oil) in DMF (1.5 mL) was added (1S,2S)-2-(dimethylamino)cyclohexanol (30 mg, 0.2 mmol) at 0° C. After stirring at 0° C. for 0.5 h, N-(5-chloro-6-(cyclopentylmethoxy)benzo[d]isoxazol-3-yl)-N-(2,4-dimethoxybenzyl)-2,4-difluorobenzenesulfonamide (60 mg, 0.1 mmol) was added and the mixture was stirred at room temperature for 16 h. The reaction was quenched with saturated aqueous NH₄Cl (20 mL), extracted with EtOAc (20 mL×3). The combined organic lawyers were washed with brine (20 mL), dried over anhydrous Na₂SO₄, filtered and concentrated in vacuo to afford the title compound (70 mg, crude) as yellow oil that required no further purification. LCMS (ESI) m/z: 716.3 [M+H]⁺.

### Step 5: N-(5-chloro-6-(cyclopentylmethoxy)benzo[d]isoxazol-3-yl)-4-(((1S,2S)-2-(dimethylamino)cyclohexyl)oxy)-2-fluorobenzenesulfonamide

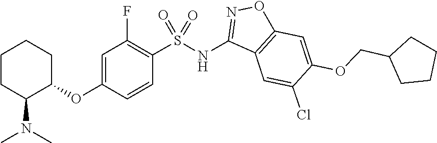 [SMILES: CN(C)[C@H]1CCCC[C@@H]1Oc1ccc(S(=O)(=O)Nc2noc3cc(OCC4CCCC4)c(Cl)cc23)c(F)c1]

A solution of N-(5-chloro-6-(cyclopentylmethoxy)benzo[d]isoxazol-3-yl)-N-(2,4-dimethoxybenzyl)-4-(((1S,2S)-2-(dimethylamino)cyclohexyl)oxy)-2-fluorobenzenesulfonamide (60 mg, 0.08 mmol) in HCOOH (3 mL) was stirred at room temperature for 16 h. The mixture was concentrated in vacuo and the crude residue was purified by reverse phase chromatography (acetonitrile 35-65%/0.2% HCOOH in water) to afford the title compound (2 mg, 4%) as a white solid. ¹H NMR (400 MHz, DMSO-d₆) δ 7.74 (t, J=8.4 Hz, 1H), 7.58 (s, 1H), 7.10 (s, 1H), 6.91-6.81 (m, 2H), 4.69-4.56 (m, 1H), 3.95 (d, J=6.8 Hz, 2H), 3.35-3.25 (m, 1H), 2.67 (s, 6H), 2.57-2.38 (m, 1H), 2.18-2.10 (m, 1H), 2.05-1.95 (m, 1H), 1.82-1.73 (m, 2H), 1.69-1.47 (m, 6H), 1.45-1.24 (m, 6H). LCMS (ESI) m/z: 566.3 [M+H]⁺.

### Example 89: N-(6-(cyclopentylmethoxy)-5-cyclopropylbenzo[d]isoxazol-3-yl)-4-(((1S,2S)-2-(dimethylamino)cyclohexyl)oxy)-2-fluorobenzenesulfonamide

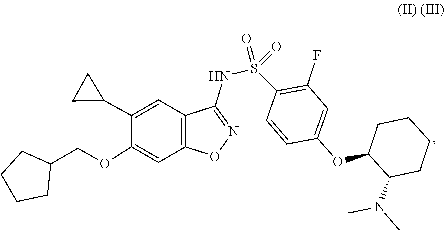 [SMILES: CN(C)[C@H]1CCCC[C@@H]1Oc1ccc(S(=O)(=O)Nc2noc3cc(OCC4CCCC4)c(C4CC4)cc23)c(F)c1]

### Step 1: 5-chloro-4-(cyclopentylmethoxy)-2-fluorobenzonitrile

To a stirred solution of NaH (183 mg, 25 mmol, 60% in mineral oil) in DMF (20 mL) was added cyclopentylmethanol (500 mg, 2.85 mmol) at 0° C. After 10 min, 5-chloro-2,4-difluorobenzonitrilee (0.72 g, 4.16 mmol) was added at 0° C. and stirred at room temperature for 16 h. The reaction was quenched with saturated aqueous NH₄Cl (30 mL), and extracted with EtOAc (30 mL×6). The combined organic layers were washed with brine (50 mL×3), dried over anhydrous Na₂SO₄, filtered and concentrated in vacuo. The crude residue was purified by silica gel chromatography (solvent gradient: 0-2% EtOAc in petroleum ether) to afford the title compound (810 mg, 77%) as a white solid. LCMS (ESI) m/z: 254.1 [M+H]⁺.

### Step 2: 4-(cyclopentylmethoxy)-5-cyclopropyl-2-fluorobenzonitrile

Following the procedure described in Example 4 and making non-critical variations as required to replace tert-butyl 5-chloro-4-(cyclopentylmethoxy)-2-fluorobenzoate with 5-chloro-4-(cyclopentylmethoxy)-2-fluorobenzonitrile, the title compound was obtained as a white solid. LCMS (ESI) m/z: 260.2 [M+H]⁺.

### Step 3: N-(6-(cyclopentylmethoxy)-5-cyclopropylbenzo[d]isoxazol-3-yl)-4-(((1S,2S)-2-(dimethylamino)cyclohexyl)oxy)-2-fluorobenzenesulfonamide

Following the procedure described in Example 88 and making non-critical variations as required to replace 5-chloro-4-(cyclopentylmethoxy)-2-fluorobenzonitrile with 4-(cyclopentylmethoxy)-5-cyclopropyl-2-fluorobenzonitrile, the title compound was obtained as a white solid. ¹H NMR (400 MHz, DMSO-d₆) δ 7.74 (t, J=8.8 Hz, 1H), 7.03 (s, 1H), 6.90 (d, J=12.4 Hz, 1H), 6.87 (s, 1H), 6.86-6.82 (m, 1H), 4.71-4.58 (m, 1H), 3.90 (d, J=6.8 Hz, 2H), 3.25-3.30 (m, 1H), 2.62 (s, 6H), 2.40-2.30 (m, 1H), 2.18-2.10 (m, 1H), 2.08-1.97 (m, 2H), 1.84-1.72 (m, 2H), 1.70-1.49 (m, 6H), 1.46-1.21 (m, 6H), 0.90-0.84 (m, 2H), 0.58-0.52 (m, 2H). LCMS (ESI) m/z: 572.2 [M+H]⁺.

### Example 90: (R)—N-(5-chloro-6-(cyclopentylmethoxy)benzo[d]isoxazol-3-yl)-4-(pyrrolidin-3-yloxy)piperidine-1-sulfonamide 2,2,2-trifluoroacetate

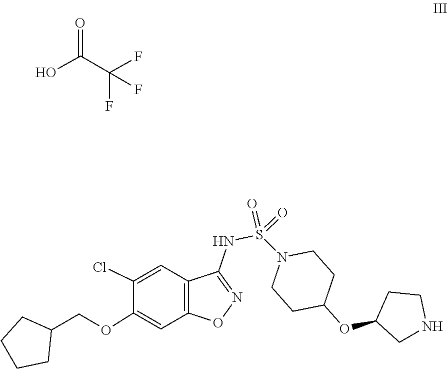 [SMILES: O=C(O)C(F)(F)F ; O=S(=O)(Nc1noc2cc(OCC3CCCC3)c(Cl)cc12)N1CCC(O[C@H]2CCNC2)CC1]

### Step 1: tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate

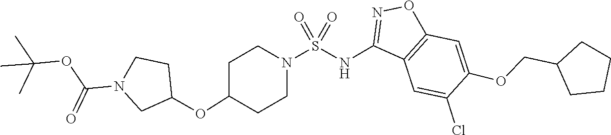 [SMILES: CC(C)(C)OC(=O)N1CCC(OC2CCN(S(=O)(=O)Nc3noc4cc(OCC5CCCC5)c(Cl)cc34)CC2)C1]

To a solution of imidazole (191 mg, 2.81 mmol) and 5-chloro-6-(cyclopentylmethoxy)benzo[d]isoxazol-3-amine (150 mg, 0.56 mmol) in DCM (6 mL) was added sulfurylchloride (0.07 mL, 0.84 mmol) at −78° C. The mixture was stirred at −78° C. for 0.5 h. Then tert-butyl 3-(piperidin-4-yloxy)pyrrolidine-1-carboxylate (456 mg, 1.69 mmol) in DCM (6 mL) was added dropwise. Then, the reaction was heated to 60° C. for 6 h. After cooling to room temperature, the reaction was diluted with water (20 mL) and extracted with DCM (30 mL×3). The combined organic layers were washed with saturated aqueous citric acid (20 mL) and brine (20 mL), dried over anhydrous Na₂SO₄, filtered and concentrated in vacuo. The crude residue was purified by silica gel chromatography (solvent gradient: 0-5% MeOH in DCM) to afford the title compound (120 mg, 36%) as colorless oil. LCMS (ESI) m/z: 499.2 [M-100+H]⁺.

### Step 2: (S)-tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate and (R)-tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate

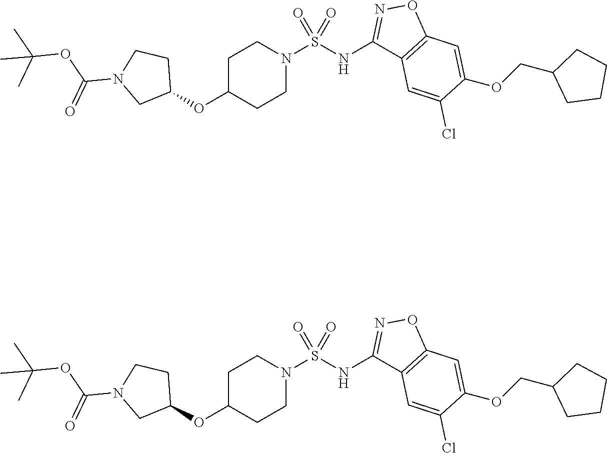 [SMILES: CC(C)(C)OC(=O)N1CC[C@H](OC2CCN(S(=O)(=O)Nc3noc4cc(OCC5CCCC5)c(Cl)cc34)CC2)C1 ; CC(C)(C)OC(=O)N1CC[C@@H](OC2CCN(S(=O)(=O)Nc3noc4cc(OCC5CCCC5)c(Cl)cc34)CC2)C1]

tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate (120 mg, 0.2 mmol) was separated by using chiral SFC (Chiralpak AD 250×30 mm I.D., 5 um; Supercritical CO₂/EtOH+0.1% NH₃·H₂O=60/40; 70 mL/min) to afford ((S)-tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate (43 mg, first peak) and (R)-tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate (42 mg, second peak) both as a white solid. Absolute configuration was arbitrarily assigned to each enantiomer. LCMS (ESI) m/z: 499.2 [M-100+H]⁺.

### Step 3: (R)—N-(5-chloro-6-(cyclopentylmethoxy)benzo[d]isoxazol-3-yl)-4-(pyrrolidin-3-yloxy)piperidine-1-sulfonamide 2,2,2-trifluoroacetate

Following the procedure described in Example 1 and making non-critical variations as required to replace tert-butyl 3-((1-(N-(5-chloro-4-(cyclopentylmethoxy)-2-fluorobenzoyl)sulfamoyl)piperidin-4-yl)oxy)azetidine-1-carboxylate with (R)-tert-butyl 3-((1-(N-(5-chloro-6-(cyclopentylmethoxy)benzo[d]isoxazol-3-yl)sulfamoyl)piperidin-4-yl)oxy)pyrrolidine-1-carboxylate, the title compound was obtained as a white solid. ¹H NMR (400 MHz, DMSO-d₆) δ 7.95 (s, 1H), 7.26 (s, 1H), 4.35-4.28 (m, 1H), 3.99 (d, J=6.8 Hz, 2H), 3.52-3.40 (m, 5H), 3.25-3.10 (m, 2H), 2.89 (t, J=10.4 Hz, 2H), 2.40-2.30 (m, 1H), 1.98-1.90 (m, 2H), 1.86-1.76 (m, 4H), 1.68-1.51 (m, 4H), 1.46-1.33 (m, 4H), LCMS (ESI) m/z: 499.1 [M+H]⁺.

### Example 91: (S)—N-(6-(cyclopentylmethoxy)-5-cyclopropylbenzo[d]isoxazol-3-yl)-4-(pyrrolidin-3-yloxy)piperidine-1-sulfonamide 2,2,2-trifluoroacetate

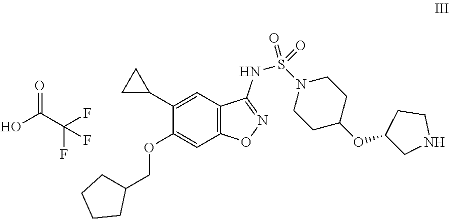

### Step 1: (S)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate and (R)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate

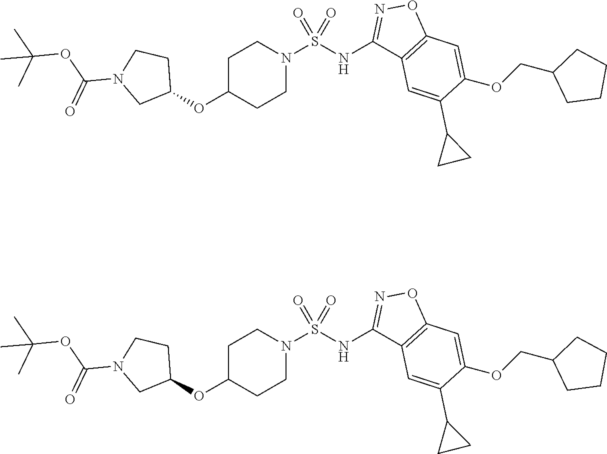 [SMILES: CC(C)(C)OC(=O)N1CC[C@H](OC2CCN(S(=O)(=O)Nc3noc4cc(OCC5CCCC5)c(C5CC5)cc34)CC2)C1 ; CC(C)(C)OC(=O)N1CC[C@@H](OC2CCN(S(=O)(=O)Nc3noc4cc(OCC5CCCC5)c(C5CC5)cc34)CC2)C1]

Following the procedure described in Example 90 and making non-critical variations as required to replace 5-chloro-6-(cyclopentylmethoxy)benzo[d]isoxazol-3-amine with 6-(cyclopentylmethoxy)-5-cyclopropylbenzo[d]isoxazol-3-amine, tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate was obtained as colorless oil. tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate (175 mg, 0.3 mmol) was separated by using chiral SFC (DAICEL CHIRALPAK AD (250 mm*30 mm, 10 um); Supercritical CO₂/MeOH+0.1% NH₃·H₂O=55/45; 70 mL/min) to afford (S)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate (70 mg, first peak) and (R)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate (72 mg, second peak) both as colorless oil. Absolute configuration was arbitrarily assigned to each enantiomer. LCMS (ESI) m/z: 505.2 [M-100+H]⁺.

### Step 2: (S)—N-(6-(cyclopentylmethoxy)-5-cyclopropylbenzo[d]isoxazol-3-yl)-4-(pyrrolidin-3-yloxy)piperidine-1-sulfonamide 2,2,2-trifluoroacetate

Following the procedure described in Example 1 and making non-critical variations as required to replace tert-butyl 3-((1-(N-(5-chloro-4-(cyclopentylmethoxy)-2-fluorobenzoyl)-sulfamoyl)piperidin-4-yl)oxy)azetidine-1-carboxylate with (S)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate, the title compound was obtained as a white solid. ¹H NMR (400 MHz, DMSO-d₆) δ 10.29-8.42 (m, 1H), 7.41 (s, 1H), 7.05 (s, 1H), 4.35-4.25 (m, 1H), 3.95 (d, J=6.8 Hz, 2H), 3.47-3.16 (m, 5H), 3.16-3.13 (m, 2H), 3.00-2.90 (m, 2H), 2.43-2.33 (m, 1H), 2.10-2.03 (m, 1H), 1.97-1.89 (m, 2H), 1.86-1.76 (m, 4H), 1.69-1.51 (m, 4H), 1.47-1.35 (m, 4H), 0.94-0.86 (m, 2H), 0.59-0.51 (m, 2H). LCMS (ESI) m/z: 505.3 [M+H]⁺.

### Example 92: (R)—N-(6-(cyclopentylmethoxy)-5-cyclopropylbenzo[d]isoxazol-3-yl)-4-(pyrrolidin-3-yloxy)piperidine-1-sulfonamide 2,2,2-trifluoroacetate

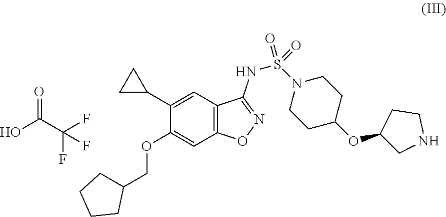

Following the procedure described in Example 91 and making non-critical variations as required to replace (S)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate with (R)-tert-butyl 3-((1-(N-(6-(cyclopentylmethoxy)-5-cyclopropylbenzo[d]isoxazol-3-yl)sulfamoyl)piperidin-4-yl)oxy)pyrrolidine-1-carboxylate, the title compound was obtained as a white solid. ¹H NMR (400 MHz, DMSO-d₆) δ 9.94-8.44 (m, 1H), 7.37 (s, 1H), 7.01 (s, 1H), 4.34-4.27 (m, 1H), 3.94 (d, J=6.8 Hz, 2H), 3.50-3.14 (m, 5H), 3.16-3.14 (m, 2H), 2.97-2.86 (m, 2H), 2.43-2.31 (m, 1H), 2.10-2.01 (m, 1H), 1.96-1.89 (m, 2H), 1.86-1.76 (m, 4H), 1.67-1.50 (m, 4H), 1.45-1.34 (m, 4H), 0.92-0.86 (m, 2H), 0.58-0.51 (m, 2H). LCMS (ESI) m/z: 505.2 [M+H]⁺.

### Example 93 and Example 94: (4aR,8aR)—N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b][1,4]oxazine-6(7H)-sulfonamide, and (4aS,8aS)—N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b][1,4]oxazine-6(7H)-sulfonamide

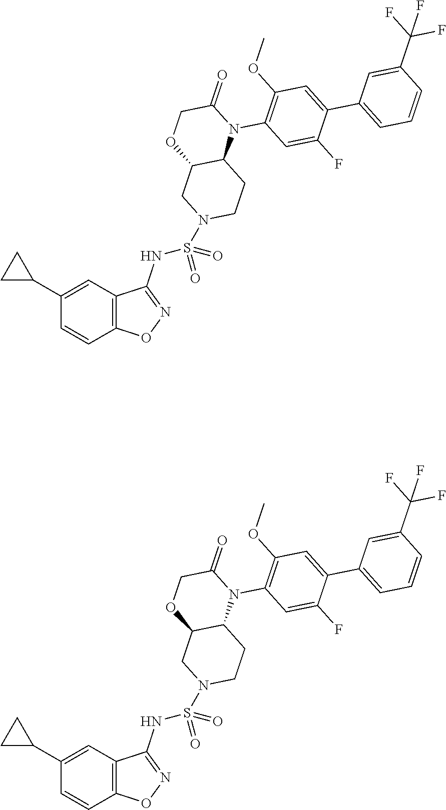 [SMILES: COc1cc(-c2cccc(C(F)(F)F)c2)c(F)cc1N1C(=O)CO[C@H]2CN(S(=O)(=O)Nc3noc4ccc(C5CC5)cc34)CC[C@@H]21 ; COc1cc(-c2cccc(C(F)(F)F)c2)c(F)cc1N1C(=O)CO[C@@H]2CN(S(=O)(=O)Nc3noc4ccc(C5CC5)cc34)CC[C@H]21]

### Step 1: trans-N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b][1,4]oxazine-6(7H)-sulfonamide

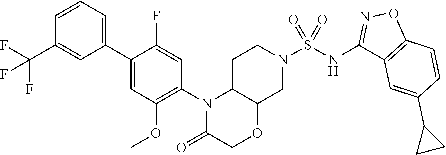 [SMILES: COc1cc(-c2cccc(C(F)(F)F)c2)c(F)cc1N1C(=O)COC2CN(S(=O)(=O)Nc3noc4ccc(C5CC5)cc34)CCC21]

Following the procedure described in Example 90 and making non-critical variations as required to replace tert-butyl 3-(piperidin-4-yloxy)pyrrolidine-1-carboxylate and 5-chloro-6-(cyclopentylmethoxy)benzo[d]isoxazol-3-amine with trans-1-(2-fluoro-5-methoxy-3′-(trifluoromethyl)-[1,1′-biphenyl]-4-yl)hexahydro-1H-pyrido[3,4-b][1,4]oxazin-2(3H)-one (Reference: WO2019/191702) and 5-cyclopropylbenzo[d]isoxazol-3-amine, the title compound was obtained as a white solid. LCMS (ESI) m/z: 661.0 [M+H]⁺.

### Step 2

trans-N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-H-pyrido[3,4-b][1,4]oxazine-6(7H)-sulfonamide (70 mg, 0.11 mmol) was separated by using chiral SFC (DAICEL CHIRALCEL OD-H (250 mm*30 mm, 5 um); Supercritical CO₂/EtOH+0.1% NH₃·H₂O=85/15; 60 mL/min) to afford (4aR,8aR)—N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b][1,4]oxazine-6(7H)-sulfonamide (13 mg, first peak) and (4aS,8aS)—N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b][1,4]oxazine-6(7H)-sulfonamide (16 mg, second peak) both as a white solid. Absolute configuration was arbitrarily assigned to each enantiomer. Example 93: ¹H NMR (400 MHz, DMSO-d₆) δ 11.40 (s, 1H), 7.92 (s, 2H), 7.84-7.71 (m, 2H), 7.67 (s, 1H), 7.58-7.50 (m, 1H), 7.43 (d, J=8.8 Hz, 1H), 7.40-7.14 (m, 2H), 4.42-4.25 (m, 2H), 4.04-3.90 (m, 1H), 3.85-3.62 (m, 6H), 3.06-2.81 (m, 2H), 2.11-2.01 (m, 1H), 1.55-1.27 (m, 2H), 1.04-0.95 (m, 2H), 0.71-0.64 (m, 2H). LCMS (ESI) m/z: 661.0 [M+H]⁺. Example 94: ¹H NMR (400 MHz, DMSO-d₆) δ 11.39 (s, 1H), 7.92 (s, 2H), 7.85-7.71 (m, 2H), 7.66 (s, 1H), 7.57-7.49 (m, 1H), 7.41 (d, J=8.8 Hz, 1H), 7.38-7.15 (m, 2H), 4.41-4.31 (m, 2H), 4.01-3.91 (m, 1H), 3.85-3.61 (m, 6H), 3.03-2.81 (m, 2H), 2.11-2.00 (m, 1H), 1.55-1.27 (m, 2H), 1.03-0.97 (m, 2H), 0.72-0.64 (m, 2H). LCMS (ESI) m/z: 661.0 [M+H]⁺.

### Example 95: N-(6-bromothiazolo[4,5-b]pyridin-2-yl)-4-(((1S,2S)-2-(dimethylamino)-cyclohexyl)amino)-2-fluorobenzenesulfonamide

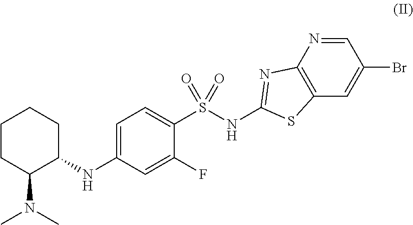 [SMILES: CN(C)[C@H]1CCCC[C@@H]1Nc1ccc(S(=O)(=O)Nc2nc3ncc(Br)cc3s2)c(F)c1]

### Step 1: (4aR,8aR)—N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b]1,4]oxazine-6(7H)-sulfonamide and (4aS,8aS)—N-(5-cyclopropylbenzo[d]isoxazol-3-yl)-1-(2-fluoro-5-methoxy-3′-(trifluoromethyl)-[1,1′-biphenyl]-4-yl)-2-oxohexahydro-1H-pyrido[3,4-b][1,4]oxazine-6(7H)-sulfonamide

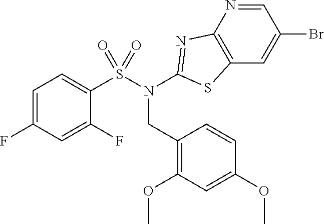 [SMILES: COc1ccc(CN(c2nc3ncc(Br)cc3s2)S(=O)(=O)c2ccc(F)cc2F)c(OC)c1]

Following the procedure described in Example 88 and making non-critical variations as required to replace 5-chloro-6-(cyclopentylmethoxy)benzo[d]isoxazol-3-amine with 6-bromothiazolo[4,5-b]pyridin-2-amine, the title compound was obtained as colorless oil. LCMS (ESI) m/z: 405.9 [M-150+H]⁺.

### Step 2: N-(6-bromothiazolo[4,5-b]pyridin-2-yl)-N-(2,4-dimethoxybenzyl)-4-(((1S,2S)-2-(dimethylamino)cyclohexyl)amino)-2-fluorobenzenesulfonamide

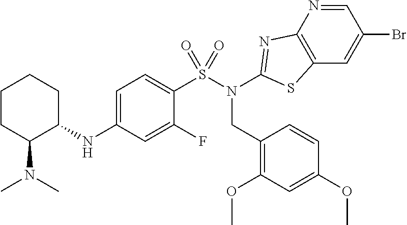 [SMILES: COc1ccc(CN(c2nc3ncc(Br)cc3s2)S(=O)(=O)c2ccc(N[C@H]3CCCC[C@@H]3N(C)C)cc2F)c(OC)c1]

To a solution of N-(6-bromothiazolo[4,5-b]pyridin-2-yl)-4-(((1S,2S)-2-(dimethylamino)cyclohexyl)amino)-2-fluorobenzenesulfonamide (40 mg, 0.07 mmol) in DMSO (1 mL) was added DIPEA (14 mg, 0.11 mmol) and (1S,2S)—N₁,N₁-dimethylcyclohexane-1,2-diamine (15 mg, 0.11 mmol) at room temperature. The reaction mixture was stirred at room temperature for 20 h. The reaction was quenched with saturated aqueous NH₄Cl (20 mL), extracted with EtOAc (30 mL×3). The combined organic layers were washed with brine (30 mL), dried over anhydrous Na₂SO₄, filtered and concentrated in vacuo to afford the title compound (48 mg, crude) as yellow oil that required no further purification. LCMS (ESI) m/z: 678.2 [M+H]⁺.

### Step 3: N-(6-bromothiazolo[4,5-b]pyridin-2-yl)-4-(((1S,2S)-2-(dimethylamino)cyclohexyl)amino)-2-fluorobenzenesulfonamide

Following the procedure described in Example 88 and making non-critical variations as required to replace N-(5-chloro-6-(cyclopentylmethoxy)benzo[d]isoxazol-3-yl)-N-(2,4-dimethoxybenzyl)-4-(((1S,2S)-2-(dimethylamino)cyclohexyl)oxy)-2-fluorobenzene sulfonamide with N-(6-bromothiazolo[4,5-b]pyridin-2-yl)-N-(2,4-dimethoxybenzyl)-4-(((1S,2S)-2-(dimethylamino)cyclohexyl)amino)-2-fluorobenzene sulfonamide, the title compound was obtained as a white solid. ¹H NMR (400 MHz, DMSO-d₆) δ 8.17 (d, J=2.4 Hz, 1H), 8.12 (d, J=2.4 Hz, 1H), 7.52 (t, J=8.8 Hz, 1H), 6.50-6.25 (m, 2H), 6.29-6.20 (m, 1H), 3.72-3.60 (m, 1H), 3.08-2.92 (m, 1H), 2.59 (s, 6H), 2.05-1.93 (m, 2H), 1.85-1.75 (m, 1H), 1.67-1.55 (m, 1H), 1.42-1.17 (m, 4H), LCMS (ESI) m/z: 529.9 [M+H]⁺.

### Example 96: N-(6-(cyclopentylmethoxy)benzo[d]thiazol-2-yl)-4-(((1S,2S)-2-(dimethyl-amino)cyclohexyl)-amino)-2-fluorobenzenesulfonamide formate

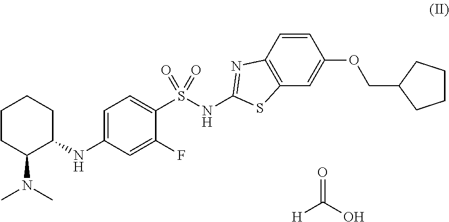 [SMILES: CN(C)[C@H]1CCCC[C@@H]1Nc1ccc(S(=O)(=O)Nc2nc3ccc(OCC4CCCC4)cc3s2)c(F)c1 ; O=CO]

### Step 1: 4-(cyclopentylmethoxy)aniline

To a stirred solution of NaH (1.32 g, 54.98 mmol, 60% in mineral oil) in DMF (20 mL) was added 4-aminophenol (2 g, 18.33 mmol) at 0° C. under nitrogen atmosphere. After stirring at 0° C. for 10 min, (bromomethyl)cyclopentane (4.48 g, 27.49 mmol) was added at 0° C. The reaction was stirred at room temperature for 16 h. The reaction was quenched with water (100 mL) and extracted with EtOAc (100 mL×3). The combined organic layers were washed with brine (100 mL), dried over anhydrous Na₂SO₄, filtered and concentrated in vacuo. The crude residue was purified by silica gel chromatography (solvent gradient: 10-20% EtOAc in petroleum ether) to afford the title compound (1.35 g, 39%) as black oil. ¹H NMR (400 MHz, CDCl₃) δ 6.77-6.74 (m, 2H), 6.66-6.63 (m, 2H), 3.76 (d, J=7.2 Hz, 2H), 3.42 (s, 2H), 2.38-2.27 (m, 1H), 1.86-1.78 (m, 2H), 1.64-1.58 (m, 4H), 1.39-1.30 (m, 2H). LCMS (ESI) m/z: 192.2 [M+H]⁺.

### Step 2: 6-(cyclopentylmethoxy)benzo[d]thiazol-2-amine

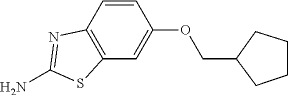 [SMILES: Nc1nc2ccc(OCC3CCCC3)cc2s1]

The solution of 4-(cyclopentylmethoxy)aniline (1.30 g, 7.06 mmol) and potassium thiocyanate (685 mg, 7.06 mmol) in acetic acid (7.5 mL) was stirred at 0° C. for 20 min. Bromine (0.36 mL, 7.06 mmol) in acetic acid (3.5 mL) was added slowly, maintaining the temperature below 10° C. Then, the mixture was stirred at room temperature for 18 h. The reaction was filtered and the filter cake was washed with acetic acid (5 mL). The filtrate was concentrated in vacuo and the crude reside was diluted with hot water (5 mL) and basified to pH>11 with NH₃·H₂O. The resulting precipitate was filtered and the filter cake was washed with water (5 mL). The filter cake was diluted with DCM (20 mL), dried over anhydrous Na₂SO₄, filtered and concentrated in vacuo. The crude residue was purified by silica gel chromatography (solvent gradient: 0-14% EtOAc in petroleum ether) to afford the title compound (800 mg, 46%) as a gray solid. ¹H NMR (400 MHz, CDCl₃) δ 7.43 (d, J=8.8 Hz, 1H), 7.13 (d, J=6.0 Hz, 1H), 6.93-6.90 (m, 1H), 5.22 (s, 2H), 3.84 (d, J=7.2 Hz, 2H), 2.41-2.33 (m, 1H), 1.89-1.81 (m, 2H), 1.68-1.58 (m, 4H), 1.41-1.33 (m, 2H). LCMS (ESI) m/z: 249.0 [M+H]⁺.

### Step 3: N-(6-(cyclopentylmethoxy)benzo[d]thiazol-2-yl)-4-(((1S,2S)-2-(dimethylamino)cyclohexyl)amino)-2-fluorobenzenesulfonamide formate

Following the procedure described in Example 95 and making non-critical variations as required to replace 6-bromothiazolo[4,5-b]pyridin-2-amine with 6-(cyclopentylmethoxy)benzo[d]thiazol-2-amine, the title compound was obtained as a white solid. ¹H NMR (400 MHz, DMSO-d₆) δ 7.49 (t, J=8.8 Hz, 1H), 7.26 (d, J=2.4 Hz, 1H), 7.16 (d, J=8.8 Hz, 1H), 6.84-6.81 (m, 1H), 6.42-6.36 (m, 3H), 3.79 (d, J=6.8 Hz, 2H), 3.47-3.40 (m, 1H), 2.82-2.75 (m, 1H), 2.42 (s, 6H), 2.33-2.24 (m, 1H), 2.02-1.92 (m, 2H), 1.79-1.71 (m, 3H), 1.61-1.48 (m, 5H), 1.34-1.20 (m, 6H). LCMS (ESI) m/z: 547.3 [M+H]⁺.

### Example 97: N-(6-(cyclopentylmethoxy)benzo[d]thiazol-2-yl)-4-(((1S,2S)-2-(dimethyl-amino)cyclohexyl)oxy)-2-fluorobenzenesulfonamide

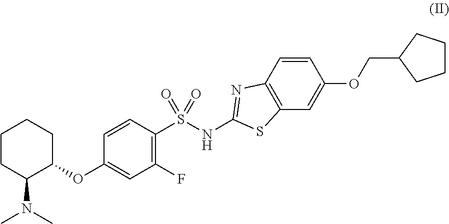 [SMILES: CN(C)[C@H]1CCCC[C@@H]1Oc1ccc(S(=O)(=O)Nc2nc3ccc(OCC4CCCC4)cc3s2)c(F)c1]

Following the procedure described in Example 88 and making non-critical variations as required to replace 5-chloro-6-(cyclopentylmethoxy)benzo[d]isoxazol-3-amine with 6-(cyclopentylmethoxy)benzo[d]thiazol-2-amine, the title compound was obtained as a white solid. ¹H NMR (400 MHz, DMSO-d₆) δ 7.73 (t, J=8.4 Hz, 1H), 7.24 (d, J=2.4 Hz, 1H), 7.17 (d, J=8.8 Hz, 1H), 6.97-6.92 (m, 1H), 6.86-6.83 (m, 1H), 6.82-6.79 (m, 1H), 4.62-4.56 (m, 1H), 3.79 (d, J=7.2 Hz, 2H), 3.09-2.98 (m, 1H), 2.47 (s, 6H), 2.30-2.21 (m, 1H), 2.11-2.08 (m, 1H), 1.93-1.90 (m, 1H), 1.78-1.72 (m, 3H), 1.63-1.47 (m, 5H), 1.37-1.20 (m, 6H). LCMS (ESI) m/z: 548.2 [M+H]⁺.

### Example 99: N-(7-chlorobenzo[d]thiazol-2-yl)-4-(((1S,2S)-2-(dimethylamino)cyclohexyl)-oxy)-2-fluorobenzenesulfonamide

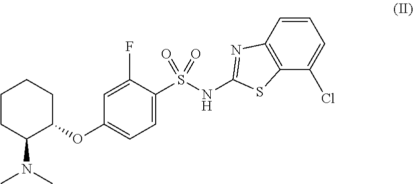 [SMILES: CN(C)[C@H]1CCCC[C@@H]1Oc1ccc(S(=O)(=O)Nc2nc3cccc(Cl)c3s2)c(F)c1]

Following the procedure described in Example 88 and making non-critical variations as required to replace 5-chloro-6-(cyclopentylmethoxy)benzo[d]isoxazol-3-amine with 7-chlorobenzo[d]thiazol-2-amine, the title compound was obtained as a white solid. ¹H NMR (400 MHz, DMSO-d₆) δ 7.76 (t, J=8.8 Hz, 1H), 7.24-7.20 (m, 1H), 7.15 (t, J=8.0 Hz, 1H), 7.03-6.97 (m, 2H), 6.92-6.87 (m, 1H), 4.72-4.64 (m, 1H), 3.43-3.42 (m, 1H), 2.67 (s, 6H), 2.21-2.12 (m, 1H), 2.07-2.00 (m, 1H), 1.83-1.73 (m, 1H), 1.67-1.60 (m, 1H), 1.47-1.20 (m, 4H). LCMS (ESI) m/z: 484.1 [M+H]⁺.

### Example 101: 5-chloro-4-(((1S,2S)-2-(dimethylamino)cyclohexyl)amino)-2-fluoro-N-(5-(2-(1-(trifluoromethyl)cyclopropyl)ethoxy)pyridin-2-yl)benzenesulfonamide

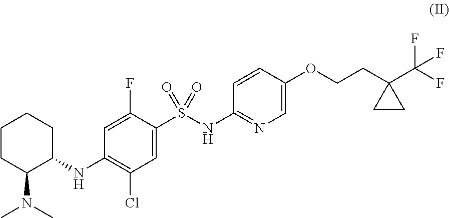 [SMILES: CN(C)[C@H]1CCCC[C@@H]1Nc1cc(F)c(S(=O)(=O)Nc2ccc(OCCC3(C(F)(F)F)CC3)cn2)cc1Cl]

### Step 1: 2-(1-(trifluoromethyl)cyclopropyl)ethanol

To a solution of LiAlH₄ (88 mg, 2.32 mmol) in THF (6 mL) was added 2-[1-(trifluoromethyl)cyclopropyl]acetic acid (300 mg, 1.78 mmol) in THF (6 mL) at room temperature. After stirring for 16 h, the reaction mixture was quenched with water (0.1 mL) and 15% aqueous NaOH solution (0.1 mL). The reaction mixture was directly dried over MgSO₄, filtered and concentrated under light vacuum to afford the title compound (260 mg, crude) as colorless oil that required no further purification. ¹H NMR (400 MHz, CDCl₃) δ 3.81 (t, J=6.0 Hz, 2H), 1.85 (t, J=7.2 Hz, 2H), 1.03-0.96 (m, 2H), 0.71-0.65 (m, 2H).

### Step 2: tert-butyl (5-(2-(1-(trifluoromethyl)cyclopropyl)ethoxy)pyridin-2-yl)carbamate

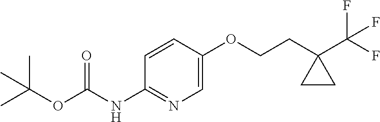 [SMILES: CC(C)(C)OC(=O)Nc1ccc(OCCC2(C(F)(F)F)CC2)cn1]

Following the procedure described in Example 1 and making non-critical variations as required to replace tert-butyl 3-hydroxyazetidine-1-carboxylate and pyridin-4-ol with tert-butyl (5-hydroxypyridin-2-yl)carbamate and 2-(1-(trifluoromethyl)cyclopropyl)ethanol, the title compound was obtained as a white solid. LCMS (ESI) m/z: 347.1 [M+H]⁺.

### Step 3: 5-(2-(1-(trifluoromethyl)cyclopropyl)ethoxy)pyridin-2-amine

Following the procedure described in Example 1 and making non-critical variations as required to replace tert-butyl 3-((1-(N-(5-chloro-4-(cyclopentylmethoxy)-2-fluorobenzoyl)sulfamoyl)piperidin-4-yl)oxy)azetidine-1-carboxylate with tert-butyl (5-(2-(1-(trifluoromethyl)cyclopropyl)ethoxy)pyridin-2-yl)carbamate, the title compound was obtained as a white solid. LCMS (ESI) m/z: 247.2 [M+H]⁺.

### Step 4: 5-chloro-4-(((1S,2S)-2-(dimethylamino)cyclohexyl)amino)-2-fluoro-N-(5-(2-(1-(trifluoromethyl)cyclopropyl)ethoxy)pyridin-2-yl)benzenesulfonamide

Following the procedure described in Example 88 and making non-critical variations as required to replace 5-chloro-6-(cyclopentylmethoxy)benzo[d]isoxazol-3-amine with 5-(2-(1-(trifluoromethyl)cyclopropyl)ethoxy)pyridin-2-amine, the title compound was obtained as a white solid. ¹H NMR (400 MHz, DMSO-d₆) δ 7.85 (s, 1H), 7.62 (d, J=6.8 Hz, 1H), 7.36 (d, J=6.8 Hz, 1H), 7.03 (d, J=8.8 Hz, 1H), 6.73 (d, J=13.2 Hz, 1H), 5.96 (d, J=3.2 Hz, 1H), 4.07-3.99 (m, 2H), 3.38-3.25 (m, 1H), 2.69-2.58 (m, 1H), 2.17 (s, 6H), 2.08-1.93 (m, 3H), 1.88-1.68 (m, 2H), 1.64-1.53 (m, 1H), 1.42-1.27 (m, 1H), 1.25-1.04 (m, 3H), 0.97-0.69 (m, 4H). LCMS (ESI) m/z. 579.1 [M+H]⁺.

### Example 102: 5-chloro-4-(((1S,2S)-2-(dimethylamino)cyclohexyl)amino)-2-fluoro-N-(1-methyl-2-((E)-3-methylbut-1-en-1-yl)-1H-pyrrolo[3,2-c]pyridin-6-yl)benzenesulfonamide

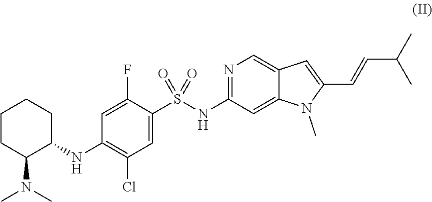 [SMILES: CC(C)/C=C/c1cc2cnc(NS(=O)(=O)c3cc(Cl)c(N[C@H]4CCCC[C@@H]4N(C)C)cc3F)cc2n1C]

### Step 1: tert-butyl 6-[bis(benzyloxycarbonyl)amino]-2-iodo-pyrrolo[3,2-c]pyridine-1-carboxylate

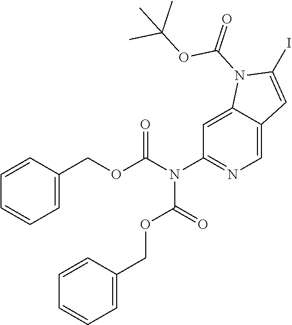 [SMILES: CC(C)(C)OC(=O)n1c(I)cc2cnc(N(C(=O)OCc3ccccc3)C(=O)OCc3ccccc3)cc21]

To a solution of tert-butyl 2-iodopyrrolo[3,2-c]pyridine-1-carboxylate (2.45 g, 7.12 mmol) and benzyl chloroformate (3.5 mL, 24.92 mmol) in DCM (50 mL) was added DIPEA (3.1 mL, 17.8 mmol) at 0° C. Then, the reaction was warmed to room temperature and stirred for 16 h. The mixture was diluted with water (50 mL), extracted with DCM (100 mL×3). The combined organic layers were washed with brine (100 mL), dried over anhydrous Na₂SO₄, filtered and concentrated in vacuo. The crude residue was purified by silica gel chromatography (solvent gradient: 0-30% EtOAc in petroleum ether) to afford the title compound (1.66 g, 37%) as yellow oil. LCMS (ESI) m/z: 628.3 [M+H]⁺.

### Step 2: benzyl N-benzyloxycarbonyl-N-(2-iodo-1H-pyrrolo[3,2-c]pyridin-6-yl)carbamate

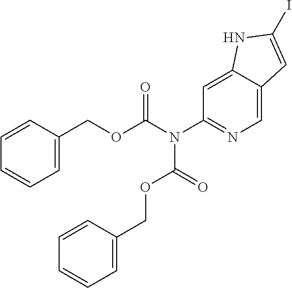 [SMILES: O=C(OCc1ccccc1)N(C(=O)OCc1ccccc1)c1cc2[nH]c(I)cc2cn1]

To a stirred solution of tert-butyl 6-[bis(benzyloxycarbonyl)amino]-2-iodo-pyrrolo[3,2-c]pyridine-1-carboxylate (1.7 g, 2.71 mmol) in DCM (30 mL) was added TFA (10 mL, 134.19 mmol) at room temperature and the reaction was stirred at room temperature for 2 h. The mixture was concentrated in vacuo, the crude residue was dissolved in DCM (100 mL), washed with 10% aqueous NaOH solution (50 mL) and brine (50 mL), the organic layer was dried over anhydrous Na₂SO₄, filtered, and concentrated in vacuo to afford the title compound (1.4 g, crude) as yellow oil that required no further purification. LCMS (ESI) m/z: 528.1 [M+H]⁺.

### Step 3: benzyl N-benzyloxycarbonyl-N-(2-iodo-1-methyl-pyrrolo[3,2-c]pyridin-6-yl)carbamate

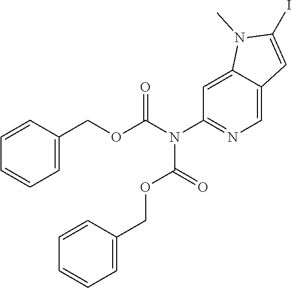 [SMILES: Cn1c(I)cc2cnc(N(C(=O)OCc3ccccc3)C(=O)OCc3ccccc3)cc21]

A mixture of benzyl N-benzyloxycarbonyl-N-(2-iodo-1H-pyrrolo[3,2-c]pyridin-6-yl)carbamate (1.39 g, 2.64 mmol) and cesium carbonate (1.29 g, 3.95 mmol) in DMF (25 mL) was stirred at room temperature for 30 min under nitrogen atmosphere. Then iodomethane (1.8 mL, 29.1 mmol) was added slowly. The reaction was stirred at room temperature for 16 h. The reaction was quenched with saturated aqueous NH₄Cl solution (40 mL) and extracted with EtOAc (100 mL×3). The combined organic layers were washed with brine (50 mL×2), dried over anhydrous Na₂SO₄, filtered and concentrated in vacuo. The crude residue was purified by silica gel chromatography (solvent gradient: 0-30% EtOAc in petroleum ether) to afford the title compound (220 mg, 15%) as a yellow solid. LCMS (ESI) m/z: 542.1 [M+H]⁺.

### Step 4: benzyl N-benzyloxycarbonyl-N-[1-methyl-2-[(E)-3-methylbut-1-enyl]pyrrolo[3,2-c]pyridin-6-yl]carbamate

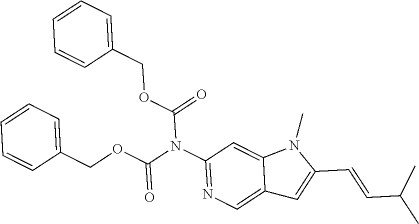 [SMILES: CC(C)/C=C/c1cc2cnc(N(C(=O)OCc3ccccc3)C(=O)OCc3ccccc3)cc2n1C]

To a solution of benzyl N-benzyloxycarbonyl-N-(2-iodo-1-methyl-pyrrolo[3,2-c]pyridin-6-yl)carbamate (350 mg, 0.65 mmol), K₂CO₃ (178 mg, 1.29 mmol) and 4,4,5,5-tetramethyl-2-[(E)-3-methylbut-1-enyl]-1,3,2-dioxaborolane (300 mg, 1.53 mmol) in 1,4-dioxane (2 mL) and water (0.2 mL) was added Pd(dppf)Cl₂ (47 mg, 0.06 mmol). The resulting mixture was stirred at 75° C. under N₂ atmosphere for 16 h. After cooling to room temperature, the reaction was diluted with water (30 mL) and extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-30% EtOAc in petroleum ether) to afford the title compound (120 mg, 38%) as yellow oil. LCMS (ESI) m/z: 484.3 [M+H]⁺.

### Step 5: (E)-1-methyl-2-(3-methylbut-1-en-1-yl)-1H-pyrrolo[3,2-c]pyridin-6-amine

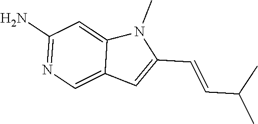 [SMILES: CC(C)/C=C/c1cc2cnc(N)cc2n1C]

A mixture of benzyl N-benzyloxycarbonyl-N-[1-methyl-2-[(E)-3-methylbut-1-enyl]pyrrolo[3,2-c]pyridin-6-yl]carbamate (190 mg, 0.39 mmol) in EtOH (13 mL), and water (7 mL) was added KOH (1.1 g, 19.65 mmol). The reaction was stirred at 100° C. for 2 h. After cooling to room temperature, the mixture was concentrated in vacuo to remove most solvent. The crude residue was diluted with water (20 mL) and extracted with EtOAc (20 mL×3). The combined organic layers were washed with brine (20 mL), dried over anhydrous Na₂SO₄, filtered and concentrated in vacuo to afford the title compound (84 mg, crude) as yellow oil that required no further purification. LCMS (ESI) m/z: 216.2 [M+H]⁺.

### Step 6: (E)-N-(2,4-dimethoxybenzyl)-1-methyl-2-(3-methylbut-1-en-1-yl)-1H-pyrrolo[3,2-c]pyridin-6-amine

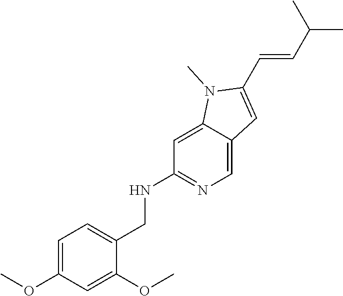 [SMILES: COc1ccc(CNc2cc3c(cn2)cc(/C=C/C(C)C)n3C)c(OC)c1]

Following the procedure described in Example 88 and making non-critical variations as required to replace 5-chloro-6-(cyclopentylmethoxy)benzo[d]isoxazol-3-amine with (E)-1-methyl-2-(3-methylbut-1-en-1-yl)-1H-pyrrolo[3,2-c]pyridin-6-amine, the title compound was obtained as yellow oil. LCMS (ESI) m/z: 366.3 [M+H]⁺.

### Step 7: (E)-5-chloro-N-(2,4-dimethoxybenzyl)-2,4-difluoro-N-(1-methyl-2-(3-methylbut-1-en-1-yl)-1H-pyrrolo[3,2-c]pyridin-6-yl)benzenesulfonamide

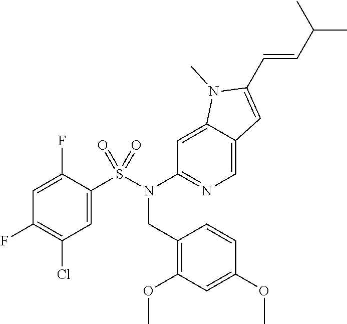 [SMILES: COc1ccc(CN(c2cc3c(cn2)cc(/C=C/C(C)C)n3C)S(=O)(=O)c2cc(Cl)c(F)cc2F)c(OC)c1]

A mixture of (E)-N-(2,4-dimethoxybenzyl)-1-methyl-2-(3-methylbut-1-en-1-yl)-1H-pyrrolo[3,2-c]pyridin-6-amine (70 mg, 0.19 mmol) in MeCN (3 mL) was added 5-chloro-2,4-difluorobenzenesulfonylchloride (94 mg, 0.38 mmol) and 1,4-diazabicyclo[2.2.2]octane (43 mg, 0.38 mmol) at room temperature. The mixture was stirred at room temperature for 16 h. The reaction was diluted with water (30 mL) and extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-50% EtOAc in petroleum ether) to afford the title compound (70 mg, 63%) as a yellow solid. LCMS (ESI) m/z: 576.2 [M+H]⁺.

### Step 8: 5-chloro-4-(((1S,2S)-2-(dimethylamino)cyclohexyl)amino)-2-fluoro-N-(1-methyl-2-((E)-3-methylbut-1-en-1-yl)-1H-pyrrolo[3,2-c]pyridin-6-yl)benzenesulfonamide

Following the procedure described in Example 95 and making non-critical variations as required to replace N-(6-bromothiazolo[4,5-b]pyridin-2-yl)-N-(2,4-dimethoxybenzyl)-2,4-difluorobenzenesulfonamide with (E)-5-chloro-N-(2,4-dimethoxybenzyl)-2,4-difluoro-N-(1-methyl-2-(3-methylbut-1-en-1-yl)-1H-pyrrolo[3,2-c]pyridin-6-yl)benzenesulfonamide, the title compound was obtained as a white solid. ¹H NMR (400 MHz, DMSO-d₆) δ 8.24 (s, 1H), 7.70 (d, J=7.2 Hz, 1H), 6.98 (s, 1H), 6.67-6.60 (m, 2H), 6.50-6.35 (m, 2H), 5.75 (d, J=2.8 Hz, 1H), 3.59 (s, 3H), 3.25-3.18 (m, 2H), 2.61-2.52 (m, 1H), 2.14 (s, 6H), 2.10-2.03 (m, 1H), 1.83-1.73 (m, 2H), 1.61-1.52 (m, 1H), 1.40-1.26 (m, 1H), 1.21-1.15 (m, 2H), 1.11-1.06 (m, 7H). LCMS (ESI) m/z: 548.3 [M+H]⁺.

### Example 103: 5-chloro-4-(((1S,2S)-2-(dimethylamino)cyclohexyl)amino)-2-fluoro-N-(5-(3-(4-(trifluoromethyl)phenyl)propoxy)pyridin-2-yl) benzenesulfonamide

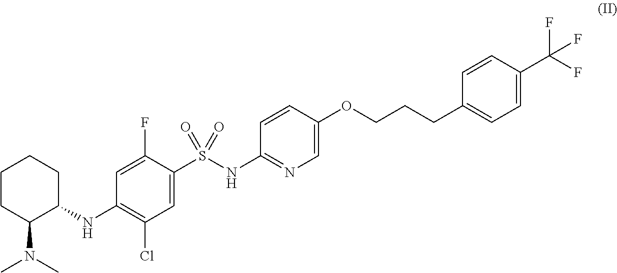 [SMILES: CN(C)[C@H]1CCCC[C@@H]1Nc1cc(F)c(S(=O)(=O)Nc2ccc(OCCCc3ccc(C(F)(F)F)cc3)cn2)cc1Cl]

### Step 1: tert-butyl (5-(3-(4-(trifluoromethyl)phenyl)propoxy)pyridin-2-yl)carbamate

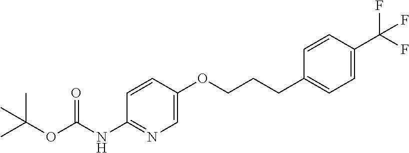 [SMILES: CC(C)(C)OC(=O)Nc1ccc(OCCCc2ccc(C(F)(F)F)cc2)cn1]

To a stirred solution of PPh₃ (390 mg, 1.48 mmol) and tert-butyl N-(5-hydroxy-2-pyridyl)carbamate (300 mg, 1.43 mmol) in THF (9 mL) was added DIAD (300 mg, 1.48 mmol) at 0° C. The mixture was stirred for 16 h at room temperature under nitrogen atmosphere. The reaction was diluted with water (50 mL) and extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-30% EtOAc in petroleum ether) to afford the title compound (320 mg, 57%) as a white solid. LCMS (ESI) m/z: 341.1 [M-56+H]⁺.

### Step 2: 5-chloro-4-(((1S,2S)-2-(dimethylamino)cyclohexyl)amino)-2-fluoro-N-(5-(3-(4-(trifluoromethyl)phenyl)propoxy)pyridin-2-yl) benzenesulfonamide

Following the procedure described in Example 101 and making non-critical variations as required to replace tert-butyl (5-(2-(1-(trifluoromethyl)cyclopropyl)ethoxy)pyridin-2-yl)carbamate with tert-butyl (5-(3-(4-(trifluoromethyl)phenyl)propoxy)pyridin-2-yl)carbamate, the title compound was obtained as a white solid. ¹H NMR (400 MHz, DMSO-d₆) δ 7.87 (d, J=2.8 Hz, 1H), 7.66-7.59 (m, 2H), 7.50-7.41 (m, 2H), 7.40-7.35 (m, 1H), 7.03 (d, J=8.4 Hz, 1H), 6.74 (d, J=13.2 Hz, 1H), 5.98-5.89 (m, 1H), 3.96 (t, J=6.4 Hz, 2H), 2.80 (t, J=7.6 Hz, 2H), 2.65-2.55 (m, 1H), 2.55-2.50 (m, 1H), 2.16 (s, 6H), 2.07-1.94 (m, 3H), 1.89-1.69 (m, 2H), 1.65-1.55 (m, 1H), 1.42-1.27 (m, 1H), 1.26-1.06 (m, 3H). LCMS (ESI) m/z: 629.3 [M+H]⁺.

### Example 104: (R)-2-(5-chloro-4-(cyclopentylmethoxy)-2-fluorobenzamido)-2-phenylacetic acid

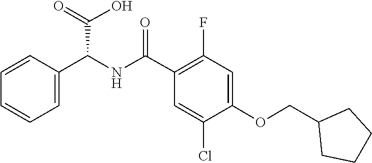 [SMILES: O=C(N[C@@H](C(=O)O)c1ccccc1)c1cc(Cl)c(OCC2CCCC2)cc1F]

### Step 1: methyl 2-(5-chloro-4-(cyclopentylmethoxy)-2-fluorobenzamido)-2-phenylacetate

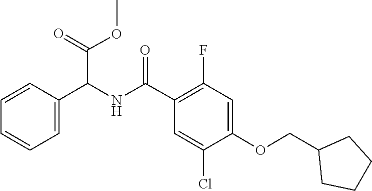 [SMILES: COC(=O)C(NC(=O)c1cc(Cl)c(OCC2CCCC2)cc1F)c1ccccc1]

Following the procedure described in Example 1 and making non-critical variations as required to replace tert-butyl 3-((1-sulfamoylpiperidin-4-yl)oxy)azetidine-1-carboxylate with methyl 2-amino-2-phenylacetate hydrochloride, the title compound was obtained as colorless oil. LCMS (ESI) m/z: 420.1 [M+H]⁺.

### Step 2: (R)-2-(5-chloro-4-(cyclopentylmethoxy)-2-fluorobenzamido)-2-phenylacetic acid

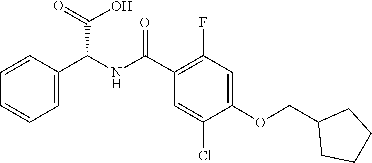 [SMILES: O=C(N[C@@H](C(=O)O)c1ccccc1)c1cc(Cl)c(OCC2CCCC2)cc1F]

Following the procedure described in Example 75 and making non-critical variations as required to replace methyl 5-cyclopropyl-2-fluorobenzoate with methyl 2-(5-chloro-4-(cyclopentylmethoxy)-2-fluorobenzamido)-2-phenylacetate, 2-(5-chloro-4-(cyclopentylmethoxy)-2-fluorobenzamido)-2-phenylacetic acid was obtained as a white solid. The enantiomer was separated by using chiral SFC (Daicel chir Alpak As (250 mm*30 mm, 10 um) Supercritical CO₂/EtOH+0.1% NH₃·H₂O=35/65; 60 mL/min) to afford the title compound (29 mg, second peak) as a white solid. Absolute configuration was arbitrarily assigned to each enantiomer. ¹H NMR (400 MHz, DMSO-d₆) δ 8.80-8.73 (m, 1H), 7.68 (d, J=7.6 Hz, 1H), 7.49-7.44 (m, 2H), 7.41-7.31 (m, 3H), 7.20 (d, J=12.8 Hz, 1H), 5.35-5.15 (m, 1H), 4.01 (d, J=6.8 Hz, 2H), 2.40-2.27 (m, 1H), 1.83-1.72 (m, 2H), 1.68-1.48 (m, 4H), 1.43-1.28 (m, 2H). LCMS (ESI) m/z: 406.0 [M+H]⁺.

### Example 107: (R)-2-(5-chloro-4-(cyclopentylmethoxy)-2-fluorobenzamido)-2-cyclohexyl-acetic acid

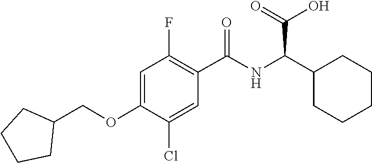 [SMILES: O=C(N[C@@H](C(=O)O)C1CCCCC1)c1cc(Cl)c(OCC2CCCC2)cc1F]

### Step 1: (R)-methyl 2-(5-chloro-4-(cyclopentylmethoxy)-2-fluorobenzamido)-2-cyclohexylacetate

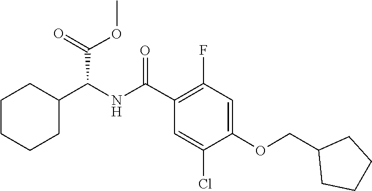 [SMILES: COC(=O)[C@H](NC(=O)c1cc(Cl)c(OCC2CCCC2)cc1F)C1CCCCC1]

To a solution of methyl (2R)-2-amino-2-cyclohexyl-acetate hydrochloride (100 mg, 0.48 mmol) and 5-chloro-4-(cyclopentylmethoxy)-2-fluoro-benzoic acid (131 mg, 0.48 mmol) in DCM (3 mL) was added DIPEA (0.26 mL, 1.44 mmol), 1-hydroxybenzotriazole (130 mg, 0.96 mmol) and 1-(3-dimethylaminopropyl)-3-ethylcarbodiimidehydrochloride (184 mg, 0.96 mmol). The reaction was stirred at room temperature for 16 h. The reaction was diluted with water (20 mL) and extracted with EtOAc (20 mL×3). The combined organic layers were washed with brine (30 mL), dried over anhydrous Na₂SO₄, filtered and concentrated in vacuo to afford the title compound (150 mg, crude) as yellow oil that required no further purification. LCMS (ESI) m/z: 426.1 [M+H]⁺.

### Step 2: (R)-2-(5-chloro-4-(cyclopentylmethoxy)-2-fluorobenzamido)-2-cyclohexylacetic acid

Following the procedure described in Example 104 and making non-critical variations as required to replace (R)-methyl 2-(5-chloro-4-(cyclopentylmethoxy)-2-fluorobenzamido)-2-phenylacetate with (R)-methyl 2-(5-chloro-4-(cyclopentylmethoxy)-2-fluorobenzamido)-2-cyclohexylacetate, the title compound was obtained as a white solid. ¹H NMR (400 MHz, DMSO-d₆) δ 12.76 (s, 1H), 8.26-8.13 (m, 1H), 7.63 (d, J=7.6 Hz, 1H), 7.18 (d, J=12.4 Hz, 1H), 4.35-4.22 (m, 1H), 4.01 (d, J=6.8 Hz, 2H), 2.41-2.24 (m, 1H), 1.86-1.51 (m, 13H), 1.42-1.30 (m, 2H), 1.22-1.08 (m, 4H). LCMS (ESI) m/z: 412.1 [M+H]⁺.

### Example 201: 4-(((1S,2S)-2-(dimethylamino)cyclohexyl)amino)-2-fluoro-N-(6-methoxybenzo[d]thiazol-2-yl)benzenesulfonamide

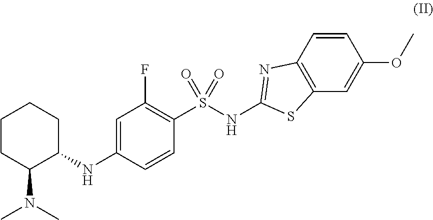 [SMILES: COc1ccc2nc(NS(=O)(=O)c3ccc(N[C@H]4CCCC[C@@H]4N(C)C)cc3F)sc2c1]

### Step 1: N-(2,4-dimethoxybenzyl)-6-methoxybenzo[d]thiazol-2-amine

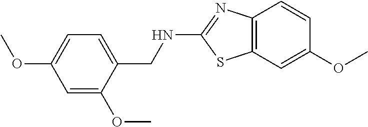

To a stirred solution of 6-methoxybenzo[d]thiazol-2-amine (500 mg, 2.77 mmol) and 2,4-dimethoxybenzaldehyde (461 g, 2.77 mmol) in DCM (20 mL) was added TiCl(Oi-Pr)₃ (6.38 mL, 6.38 mmol, 1 M in hexane) in one portion under nitrogen atmosphere. The solution was stirred for 10 min before the portion wise addition of NaBH(OAc)₃ (2.94 g, 13.87 mmol) at 0° C. The reaction was stirred at room temperature for 16 h. The reaction was quenched with saturated aqueous NaHCO₃ solution (100 mL), extracted with DCM (100 mL×3). The combined organic lawyers were washed with brine (100 mL), dried over anhydrous Na₂SO₄, filtered and concentrated in vacuo. The crude residue was purified by silica gel chromatography (solvent gradient: 0-50% EtOAc in petroleum ether) to afford the title compound (620 g, 61%) as a white solid. LCMS (ESI) m/z: 331.1 [M+H]⁺.

### Step 2: N-(2,4-dimethoxybenzyl)-2,4-difluoro-N-(6-methoxybenzo[d]thiazol-2-yl)benzenesulfonamide

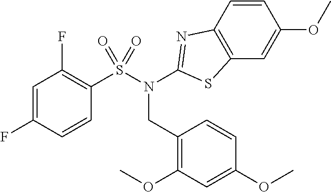 [SMILES: COc1ccc(CN(c2nc3ccc(OC)cc3s2)S(=O)(=O)c2ccc(F)cc2F)c(OC)c1]

To a solution of N-(2,4-dimethoxybenzyl)-6-methoxybenzo[d]thiazol-2-amine (350 mg, 1.06 mmol) in THF (7 mL) was added LiHMDS (1.27 mL, 1.27 mmol, 1 M) at −78° C. The reaction was stirred for 30 min at 0° C. and a solution of 2,4-difluorobenzenesulfonylchloride (450 mg, 2.12 mmol) in THF (4 mL) was added dropwise at −78° C. After stirring at room temperature for 16 h, the reaction was diluted with aqueous NH₄Cl solution (15 mL) and extracted with EtOAc (20 mL×3). The combined organic lawyers were washed with brine (30 mL), dried over anhydrous Na₂SO₄, filtered and concentrated in vacuo. The crude residue was purified by silica gel chromatography (solvent gradient: 10-30% EtOAc in petroleum ether) to afford the title compound (100 mg, 19%) as a white solid. LCMS (ESI) m/z: 507.1 [M+H]⁺.

### Step 3: N-(2,4-dimethoxybenzyl)-4-(((1S,2S)-2-(dimethylamino)cyclohexyl) amino)-2-fluoro-N-(6-methoxybenzo[d]thiazol-2-yl)benzenesulfonamide

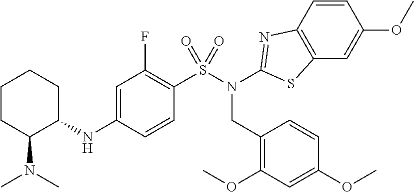 [SMILES: COc1ccc(CN(c2nc3ccc(OC)cc3s2)S(=O)(=O)c2ccc(N[C@H]3CCCC[C@@H]3N(C)C)cc2F)c(OC)c1]

To a stirred solution of (1S,2S)—N¹,N¹-dimethylcyclohexane-1,2-diamine (140 mg, 1 mmol) and DIPEA (127 mg, 1.0 mmol) in DMSO (2 mL) was added N-(2,4-dimethoxybenzyl)-2,4-difluoro-N-(6-methoxybenzo[d]thiazol-2-yl)benzenesulfonamide (100 mg, 0.2 mmol). The mixture was stirred at 60° C. for 16 h under nitrogen atmosphere. After cooling to room temperature, the reaction was quenched with sat. aq. NH₄Cl (20 mL), extracted with EtOAc (20 mL×3). The combined organic lawyers were washed with brine (20 mL), dried over anhydrous Na₂SO₄, filtered and concentrated in vacuo. The crude residue was purified by prep-TLC (PE:EtOAc=1:1) to afford the title compound (110 mg, 88%) as a white solid. LCMS (ESI) m/z: 629.3 [M+H]⁺.

### Step 4: 4-(((1S,2S)-2-(dimethylamino)cyclohexyl)amino)-2-fluoro-N-(6-methoxybenzo[d]thiazol-2-yl)benzenesulfonamide formate

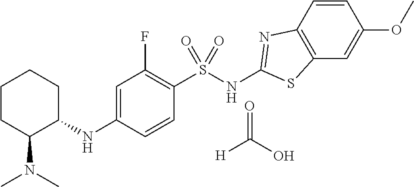

A solution of N-(2,4-dimethoxybenzyl)-4-(((1S,2S)-2-(dimethylamino)cyclohexyl) amino)-2-fluoro-N-(6-methoxybenzo[d]thiazol-2-yl)benzenesulfonamide (110 mg, 0.17 mmol) in formic acid (3 mL) was stirred at room temperature for 16 h. The mixture was concentrated in vacuo and the crude residue was purified by reverse phase chromatography (acetonitrile 15-45%/0.2% formic acid in water) to afford the title compound (13 mg, 17%) as a white solid. ¹H NMR (400 MHz, DMSO-d₆) δ 8.16 (s, 1H), 7.53-7.45 (m, 1H), 7.25 (d, J=2.8 Hz, 1H), 7.18 (d, J=8.8 Hz, 1H), 6.85-6.68 (m, 1H), 6.45-6.30 (m, 2H), 3.72 (s, 3H), 3.58-3.47 (m, 1H), 2.85-2.76 (m, 1H), 2.50 (s, 3H), 2.43 (s, 3H), 2.06-1.85 (m, 2H), 1.82-1.73 (m, 1H), 1.65-1.50 (m, 1H), 1.36-1.10 (m, 3H), 1.10-1.02 (m, 1H). LCMS (ESI) m/z: 479.1 [M+H]⁺.

### Example 202: 5-chloro-N-(6-(cyclopentylmethoxy)benzo[d]thiazol-2-yl)-4-(((1S,2S)-2-(dimethylamino)cyclohexyl)amino)-2-fluorobenzenesulfonamide

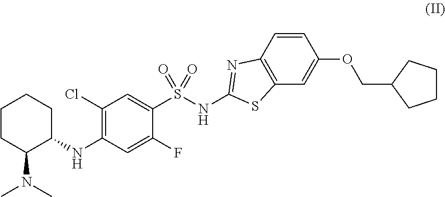 [SMILES: CN(C)[C@H]1CCCC[C@@H]1Nc1cc(F)c(S(=O)(=O)Nc2nc3ccc(OCC4CCCC4)cc3s2)cc1Cl]

### Step 1: 4-(cyclopentylmethoxy)aniline

To a stirred solution of NaH (1.32 g, 54.98 mmol, 60% in mineral oil) in DMF (20 mL) was added 4-aminophenol (2 g, 18.33 mmol) at 0° C. under nitrogen atmosphere. After stirring at 0° C. for 10 min, (bromomethyl)cyclopentane (4.48 g, 27.49 mmol) was added at 0° C. The reaction was stirred at room temperature for 16 h. The reaction was quenched with water (100 mL) and extracted with EtOAc (100 mL×3). The combined organic lawyers were washed with brine (100 mL), dried over anhydrous Na₂SO₄, filtered and concentrated in vacuo. The crude residue was purified by silica gel chromatography (solvent gradient: 10-20% EtOAc in petroleum ether) to afford the title compound (1.35 g, 39%) as black oil. ¹H NMR (400 MHz, CDCl₃) δ 6.77-6.74 (m, 2H), 6.66-6.63 (m, 2H), 3.76 (d, J=7.2 Hz, 2H), 3.42 (s, 2H), 2.38-2.27 (m, 1H), 1.86-1.78 (m, 2H), 1.64-1.58 (m, 4H), 1.39-1.30 (m, 2H). LCMS (ESI) m/z: 192.2 [M+H]⁺.

### Step 2: 6-(cyclopentylmethoxy)benzo[d]thiazol-2-amine

The solution of 4-(cyclopentylmethoxy)aniline (1.30 g, 7.06 mmol) and potassium thiocyanate (685 mg, 7.06 mmol) in acetic acid (7.5 mL) was stirred at 0° C. for 20 min. Bromine (0.36 mL, 7.06 mmol) in acetic acid (3.5 mL) was added slowly and kept the temperature below 10° C. Then, the mixture was stirred at room temperature for 18 h. The reaction was filtered and the filter cake was washed with acetic acid (5 mL). The filtrate was concentrated in vacuo and the crude reside was diluted with hot water (5 mL) and basified to pH>11 with ammonium hydroxide. The resulting precipitate was filtered and the filter cake was washed with water (5 mL). The filter cake was diluted with DCM (20 mL), dried over anhydrous Na₂SO₄, filtered and concentrated in vacuo. The crude residue was purified by silica gel chromatography (solvent gradient: 0-14% EtOAc in petroleum ether) to afford the title compound (800 mg, 46%) as a gray solid. ¹H NMR (400 MHz, CDCl₃) δ 7.43 (d, J=8.8 Hz, 1H), 7.13 (d, J=6.0 Hz, 1H), 6.93-6.90 (m, 1H), 5.22 (s, 2H), 3.84 (d, J=7.2 Hz, 2H), 2.41-2.33 (m, 1H), 1.89-1.81 (m, 2H), 1.68-1.58 (m, 4H), 1.41-1.33 (m, 2H). LCMS (ESI) m/z: 249.0 [M+H]⁺.

### Step 3: 5-chloro-N-(6-(cyclopentylmethoxy)benzo[d]thiazol-2-yl)-4-(((1S,2S)-2-(dimethylamino)cyclohexyl)amino)-2-fluorobenzenesulfonamide

Following the procedure described in Example 201 and making non-critical variations as required to replace difluorobenzenesulfonylchloride with 5-chloro-2,4-difluorobenzene-1-sulfonyl chloride, 6-methoxybenzo[d]thiazol-2-amine with 6-(cyclopentylmethoxy)benzo[d]thiazol-2-amine, the title compound was obtained as a white solid. ¹H NMR (400 MHz, DMSO-d₆) δ 13.13 (s, 1H), 8.69 (s, 1H), 7.66 (d, J=7.2 Hz, 1H), 7.46 (d, J=2.4 Hz, 1H), 7.22 (d, J=8.8 Hz, 1H), 7.07 (d, J=13.2 Hz, 1H), 7.01-6.98 (m, 1H), 6.26 (d, J=10.4 Hz, 1H), 3.98-3.92 (m, 1H), 3.83 (d, J=6.8 Hz, 2H), 3.58-3.47 (m, 1H), 2.75 (s, 3H), 2.60 (s, 3H), 2.32-2.23 (m, 1H), 2.10-2.02 (m, 1H), 1.91-1.72 (m, 4H), 1.63-1.49 (m, 5H), 1.43-1.23 (m, 6H). LCMS (ESI) m/z: 581.2 [M+H]⁺.

### Example 203: 4-(((1S,2S)-2-(dimethylamino)cyclohexyl)amino)-2-fluoro-N-(6-(trifluoromethoxy)benzo[d]thiazol-2-yl)benzenesulfonamide

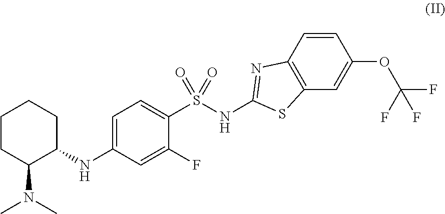 [SMILES: CN(C)[C@H]1CCCC[C@@H]1Nc1ccc(S(=O)(=O)Nc2nc3ccc(OC(F)(F)F)cc3s2)c(F)c1]

Following the procedure described in Example 201 and making non-critical variations as required to replace 6-methoxybenzo[d]thiazol-2-amine with 6-(trifluoromethoxy)-benzo[d]thiazol-2-amine, the title compound was obtained as a white solid. ¹H NMR (400 MHz, DMSO-d₆) δ 7.64 (s, 1H), 7.56-4.47 (m, 1H), 7.30 (d, J=8.8 Hz, 1H), 7.14-7.07 (m, 1H), 6.46-6.31 (m, 3H), 3.68-3.60 (m, 1H), 3.12-3.00 (m, 1H), 2.61 (s, 6H), 2.06-1.94 (m, 2H), 1.82-1.73 (m, 1H), 1.66-1.55 (m, 1H), 1.46-1.08 (m, 4H). LCMS (ESI) m/z: 533.2 [M+H]⁺.

### Example 204: 4-(cyclopentylmethoxy)-5-cyclopropyl-N-((4-(((1S,2S)-2-(dimethylamino)cyclohexyl)amino)-2,6-difluorophenyl)sulfonyl)-2-fluorobenzamide

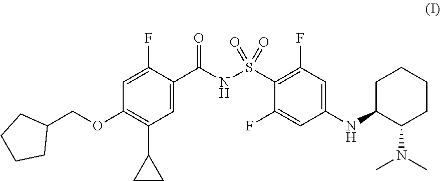 [SMILES: CN(C)[C@H]1CCCC[C@@H]1Nc1cc(F)c(S(=O)(=O)NC(=O)c2cc(C3CC3)c(OCC3CCCC3)cc2F)c(F)c1]

### Step 1: tert-butyl 2,4-dimethoxybenzyl((4-(((1S,2S)-2-(dimethylamino)cyclohexyl)-amino)-2,6-difluorophenyl)sulfonyl)carbamate

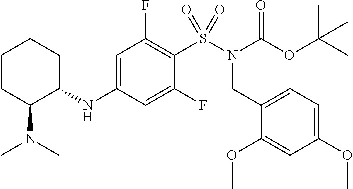 [SMILES: COc1ccc(CN(C(=O)OC(C)(C)C)S(=O)(=O)c2c(F)cc(N[C@H]3CCCC[C@@H]3N(C)C)cc2F)c(OC)c1]

To a stirred mixture of tert-butyl 2,4-dimethoxybenzyl((2,4,6-trifluorophenyl)sulfonyl)carbamate (450 mg, 0.98 mmol) and DIPEA (0.32 mL, 1.95 mmol) in DMF (5 mL) was added (1S,2S)—N¹,N¹-dimethylcyclohexane-1,2-diamine (138 mg, 0.98 mmol). After stirring at 40° C. for 2 h, the mixture was diluted with ethyl acetate (150 mL) and washed with water (50 mL×4). The organic layer was washed with brine (50 mL), dried over anhydrous NaSO₄, filtered and concentrated in vacuo. The residue was purified by prep-TLC (10% MeOH in DCM) to afford the title compound (180 mg, 32%) as yellow oil. LCMS (ESI) m/z: 584.1 [M+H]⁺.

### Step 2: 4-(((1S,2S)-2-(dimethylamino)cyclohexyl)amino)-2,6-difluorobenzenesulfonamide

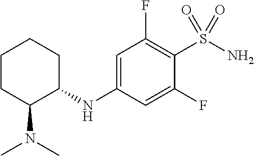 [SMILES: CN(C)[C@H]1CCCC[C@@H]1Nc1cc(F)c(S(N)(=O)=O)c(F)c1]

To a stirred solution of tert-butyl 2,4-dimethoxybenzyl((4-(((1S,2S)-2-(dimethyl-amino)cyclohexyl)amino)-2,6-difluorophenyl)sulfonyl)carbamate (180 mg, 0.31 mmol) in DCM (5 mL) was added TFA (0.5 mL, 0.31 mmol) and triethylsilane (0.51 mL, 3.22 mmol) at 20° C. After stirring at 20° C. for 1 h, the reaction was diluted with H₂O (20 mL) and extracted with DCM (20 mL×2). The combined organic layers were washed with brine (25 mL), dried over anhydrous Na₂SO₄, filtered and concentrated in vacuo. The crude residue was purified by pre-TLC (10% MeOH in DCM) to afford the title compound (100 mg, 97%) as a white solid. LCMS (ESI) m/z: 333.9 [M+H]⁺.

### Step 3: 4-(cyclopentylmethoxy)-5-cyclopropyl-N-((4-(((1S,2S)-2-(dimethylamino)-cyclohexyl)amino)-2,6-difluorophenyl)sulfonyl)-2-fluorobenzamide

A mixture of 4-(((1S,2S)-2-(dimethylamino)cyclohexyl)amino)-2,6-difluorobenzenesulfonamide (30 mg, 0.09 mmol) and 4-(cyclopentylmethoxy)-5-cyclopropyl-2-fluorobenzoic acid (27 mg, 0.10 mmol), DMAP (11 mg, 0.09 mmol) and EDCI (19 mg, 0.099 mmol) in DCM (5 mL) was stirred at room temperature for 16 h under nitrogen atmosphere. The reaction was quenched with 10% aqueous citric acid (15 mL) and extracted with DCM (20 mL×2). The combined organic lawyers were dried over anhydrous Na₂SO₄, filtered and concentrated in vacuo. The crude residue was purified by reverse phase chromatography (acetonitrile 35-65%/(0.2% formic acid) in water) to afford the title compound (10 mg, 16%) as a white solid. ¹H NMR (400 MHz, CD₃OD) δ 7.35 (d, J=8.8 Hz, 1H), 6.60 (d, J=13.2 Hz, 1H), 6.35 (d, J=11.6 Hz, 2H), 3.89 (d, J=6.4 Hz, 2H), 3.75-3.65 (m, 1H), 3.18-3.10 (m, 1H), 2.78 (s, 6H), 2.46-2.35 (m, 1H), 2.19-2.08 (m, 2H), 2.05-1.52 (m, 1OH), 1.48-1.36 (m, 4H), 1.29-1.23 (m, 1H), 0.87-0.83 (m, 2H), 0.67-0.63 (m, 2H). LCMS (ESI) m/z: 594.1 [M+H]⁺.

### Example 205: 5-chloro-4-(cyclopentylmethoxy)-N-((4-(3-(dimethylamino)piperidin-1-yl)-2-fluorophenyl)sulfonyl)-2-fluorobenzamide

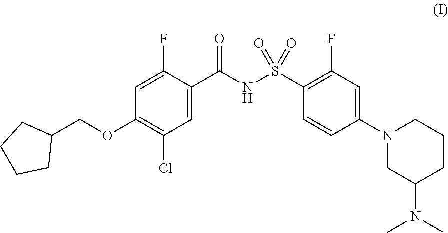 [SMILES: CN(C)C1CCCN(c2ccc(S(=O)(=O)NC(=O)c3cc(Cl)c(OCC4CCCC4)cc3F)c(F)c2)C1]

### Step 1: tert-butyl (2,4-difluorophenyl)sulfonyl(2,4-dimethoxybenzyl)carbamate

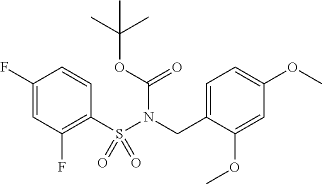 [SMILES: COc1ccc(CN(C(=O)OC(C)(C)C)S(=O)(=O)c2ccc(F)cc2F)c(OC)c1]

To a stirred solution of 2,4-dimethoxybenzylamine (7.87 g, 47.04 mmol) and pyridine (19 mL, 235.18 mmol) in DCM (220 mL) was added 2,4-difluorobenzenesulfonylchloride (10.0 g, 47.04 mmol) at 0° C. Then the reaction was stirred at room temperature for 1 h. Boc₂O (50.85 g, 233 mmol) and DMAP (5.69 g, 46.6 mmol) were added to the mixture. The reaction was stirred at 40° C. for 16 h. After cooling to room temperature, the reaction was diluted with water (100 mL) and extracted with DCM (150 mL×3). The combined organic lawyers were washed with brine (100 mL), dried over anhydrous Na₂SO₄, filtered and concentrated in vacuo. The crude residue was purified by silica gel chromatography (solvent gradient: 0-15% EtOAc in petroleum ether) to afford the title compound (15 g, 73%) as a yellow solid. LCMS (ESI) m/z: 466.1 [M+Na]⁺.

### Step 2: 5-chloro-4-(cyclopentylmethoxy)-N-((4-(3-(dimethylamino)piperidin-1-yl)-2-fluorophenyl)sulfonyl)-2-fluorobenzamide

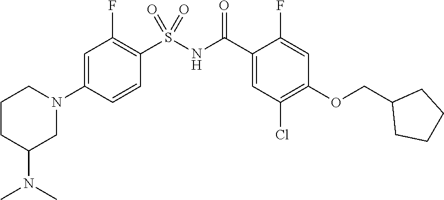 [SMILES: CN(C)C1CCCN(c2ccc(S(=O)(=O)NC(=O)c3cc(Cl)c(OCC4CCCC4)cc3F)c(F)c2)C1]

Following the procedure described in Example 204 and making non-critical variations as required to replace tert-butyl 2,4-dimethoxybenzyl((2,4,6-trifluorophenyl)sulfonyl)carbamate with tert-butyl (2,4-difluorophenyl)sulfonyl(2,4-dimethoxybenzyl)carbamate, 4-(cyclopentylmethoxy)-5-cyclopropyl-2-fluorobenzoic acid with 5-chloro-4-(cyclopentylmethoxy)-2-fluorobenzoic acid, the title compound was obtained as a white solid. ¹H NMR (400 MHz, DMSO-d₆) δ 7.75 (d, J=8.0 Hz, 1H), 7.65-7.55 (m, 1H), 6.94 (d, J=12.0 Hz, 1H), 6.78-6.70 (m, 2H), 3.98-3.90 (m, 1H), 3.94 (d, J=6.8 Hz, 2H), 3.78-3.69 (m, 1H), 3.26-3.15 (m, 1H), 3.09-2.98 (m, 1H), 2.87-2.81 (m, 1H), 2.78 (s, 6H), 2.36-2.25 (m, 1H), 2.09-2.01 (m, 1H), 1.85-1.70 (m, 3H), 1.66-1.45 (m, 6H), 1.38-1.29 (m, 2H). LCMS (ESI) m/z: 556.1 [M+H]⁺.

### Example 206 5-chloro-4-(cyclopentylmethoxy)-N-((4-(((1S,2S)-2-(dimethylamino)-cyclohexyl)amino)-2,6-difluorophenyl)sulfonyl)-2-fluorobenzamide

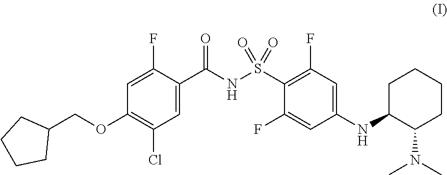 [SMILES: CN(C)[C@H]1CCCC[C@@H]1Nc1cc(F)c(S(=O)(=O)NC(=O)c2cc(Cl)c(OCC3CCCC3)cc2F)c(F)c1]

Following the procedure described in Example 204 and making non-critical variations as required to replace 4-(cyclopentylmethoxy)-5-cyclopropyl-2-fluorobenzoic acid with 5-chloro-4-(cyclopentylmethoxy)-2-fluorobenzoic acid, the title compound was obtained as a white solid. ¹H NMR (400 MHz, DMSO-d₆) δ 7.75 (d, J=8.0 Hz, 1H), 6.94 (d, J=12.4 Hz, 1H), 6.29 (d, J=11.6 Hz, 2H), 6.22 (d, J=10.0 Hz, 1H), 3.94 (d, J=6.8 Hz, 2H), 3.79-3.61 (m, 1H), 3.10-2.93 (m, 1H), 2.62 (s, 6H), 2.37-2.26 (m, 1H), 2.05-1.93 (m, 2H), 1.82-1.71 (m, 3H), 1.64-1.50 (m, 5H), 1.39-1.20 (m, 5H), 1.18-1.02 (m, 1H). LCMS (ESI) m/z: 588.1 [M+H]⁺.

### Example 207: 4-(cyclopentylmethoxy)-5-cyclopropyl-N-((4-(3-(dimethylamino)piperidin-1-yl)-2,6-difluorophenyl)sulfonyl)-2-fluorobenzamide

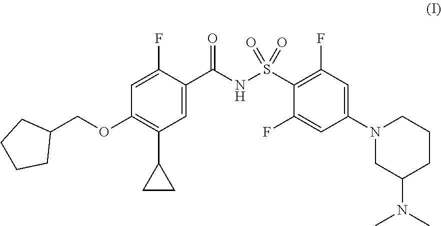 [SMILES: CN(C)C1CCCN(c2cc(F)c(S(=O)(=O)NC(=O)c3cc(C4CC4)c(OCC4CCCC4)cc3F)c(F)c2)C1]

Following the procedure described in Example 204 and making non-critical variations as required to replace (1S,2S)—N¹,N¹-dimethylcyclohexane-1,2-diamine with N,N-dimethyl-piperidin-3-amine, the title compound was obtained as a white solid. ¹H NMR (400 MHz, DMSO-d) δ 7.22 (d, J=8.8 Hz, 1H), 6.68 (d, J=12.8 Hz, 1H), 6.58 (d, J=12.4 Hz, 2H), 4.00-3.90 (m, 1H), 3.88 (d, J=6.8 Hz, 2H), 3.78 (m, 1H), 3.20-3.10 (m, 1H), 3.07-2.97 (m, 1H), 2.86-2.78 (m, 1H), 2.75 (s, 6H), 2.37-2.25 (m, 1H), 2.09-1.92 (m, 2H), 1.84-1.71 (m, 3H), 1.67-1.46 (m, 6H), 1.45-1.32 (m, 2H), 0.89-0.79 (m, 2H), 0.59-0.49 (m, 2H). LCMS (ESI) m/z: 580.3 [M+H]⁺.

### Example 208: 5-chloro-N-((4-(cyclohexylamino)-2-fluorophenyl)sulfonyl)-4-(cyclopentylmethoxy)-2-fluorobenzamide

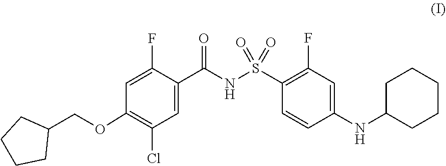 [SMILES: O=C(NS(=O)(=O)c1ccc(NC2CCCCC2)cc1F)c1cc(Cl)c(OCC2CCCC2)cc1F]

Following the procedure described in Example 205 and making non-critical variations as required to replace N,N-dimethylpiperidin-3-amine with cyclohexanamine, the title compound was obtained as a white solid. ¹H NMR (400 MHz, DMSO-d₆) δ 12.39 (s, 1H), 7.63 (d, J=7.6 Hz, 1H), 7.58-7.48 (m, 1H), 7.19 (d, J=12.4 Hz, 1H), 6.91 (d, J=7.2 Hz, 1H), 6.51-6.39 (m, 2H), 4.01 (d, J=6.8 Hz, 2H), 3.30-3.20 (m, 1H), 2.38-2.27 (m, 1H), 1.92-1.84 (m, 2H), 1.80-1.65 (m, 4H), 1.64-1.49 (m, 5H), 1.40-1.28 (m, 4H), 1.24-1.13 (m, 3H). LCMS (ESI) m/z: 527.0 [M+H]⁺.

### Example 209 5-chloro-4-(cyclopentylmethoxy)-N-((4-(3-(dimethylamino)piperidin-1-yl)phenyl)sulfonyl)-2-fluorobenzamide

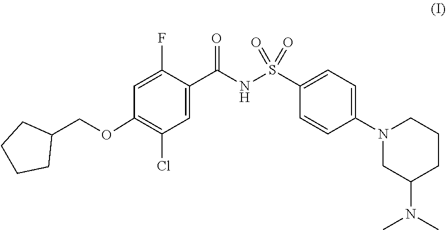 [SMILES: CN(C)C1CCCN(c2ccc(S(=O)(=O)NC(=O)c3cc(Cl)c(OCC4CCCC4)cc3F)cc2)C1]

### Step 1: 4-(3-(dimethylamino)piperidin-1-yl)benzenesulfonamide

A solution of N,N-dimethylpiperidin-3-amine (439 mg, 3.4 mmol) and 4-fluorobenzenesulfonamide (500 mg, 2.9 mmol) in DMSO (27 mL) was stirred at 100° C. for 16 h. After cooling to room temperature, the reaction was purified by reverse phase chromatography (acetonitrile 10-40%/0.05% NH₃·H₂O in water) to afford the title compound (210 mg, 26%) as a yellow solid. LCMS (ESI) m/z: 284.2 [M+H]⁺.

### Step 2: 5-chloro-4-(cyclopentylmethoxy)-N-((4-(3-(dimethylamino)piperidin-1-yl)phenyl)sulfonyl)-2-fluorobenzamide

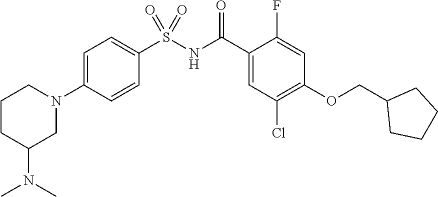 [SMILES: CN(C)C1CCCN(c2ccc(S(=O)(=O)NC(=O)c3cc(Cl)c(OCC4CCCC4)cc3F)cc2)C1]

Following the procedure described in Example 205 and making non-critical variations as required to replace 4-(3-(dimethylamino)piperidin-1-yl)-2-fluorobenzenesulfonamide with 4-(3-(dimethylamino)piperidin-1-yl)benzenesulfonamide, the title compound was obtained as a white solid. LCMS (ESI) m/z: 538.2 [M+H]⁺.

### Step 3

5-chloro-4-(cyclopentylmethoxy)-N-((4-(3-(dimethylamino)piperidin-1-yl)phenyl)sulfonyl)-2-fluorobenzamide (200 mg, 0.37 mmol) was separated by using chiral SFC (Chiralpak PAK-AS (250 mm*30 mm, 5 um), Supercritical CO₂/MeOH+0.1% NH₃·H₂O=50/50; 80 mL/min) to afford (S)-5-chloro-4-(cyclopentylmethoxy)-N-((4-(3-(dimethylamino)piperidin-1-yl)phenyl)sulfonyl)-2-fluorobenzamide (50 mg, second peak) as a white solid. Absolute configuration was arbitrarily assigned to each enantiomer. ¹H NMR (400 MHz, DMSO-d₆) δ ¹H NMR (400 MHz, DMSO-d₆) δ 7.73 (d, J=8.0 Hz, 1H), 7.66 (d, J=8.8 Hz, 2H), 6.97-6.92 (m, 3H), 3.94 (d, J=6.8 Hz, 2H), 3.94-3.85 (m, 1H), 3.74-3.64 (m, 1H), 3.12-3.02 (m, 1H), 3.01-2.90 (m, 1H), 2.83-2.75 (m, 1H), 2.73 (s, 6H), 2.35-2.27 (m, 1H), 2.08-2.00 (m, 1H), 1.84-1.70 (m, 3H), 1.65-1.42 (m, 6H), 1.39-1.27 (m, 2H). LCMS (ESI) m/z: 538.1 [M+H]⁺.

### Example 212; and Example 213: (S)-5-chloro-4-(cyclopentylmethoxy)-2-fluoro-N-((3-(piperidin-4-yloxy)pyrrolidin-1-yl)sulfonyl)benzamide, and (R)-5-chloro-4-(cyclopentylmethoxy)-2-fluoro-N-((3-(piperidin-4-yloxy)pyrrolidin-1-yl)sulfonyl)benzamide

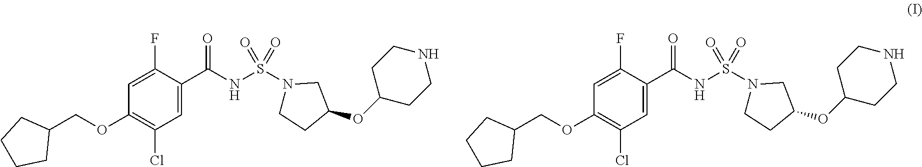 [SMILES: O=C(NS(=O)(=O)N1CC[C@H](OC2CCNCC2)C1)c1cc(Cl)c(OCC2CCCC2)cc1F ; O=C(NS(=O)(=O)N1CC[C@@H](OC2CCNCC2)C1)c1cc(Cl)c(OCC2CCCC2)cc1F]

### Step 1: benzyl 4-((1-(tert-butoxycarbonyl)pyrrolidin-3-yl)oxy)piperidine-1-carboxylate

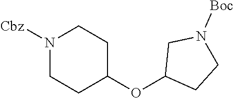 [SMILES: CC(C)(C)OC(=O)N1CCC(OC2CCN(C(=O)OCc3ccccc3)CC2)C1]

Benzyl chloroformate (0.22 mL, 1.63 mmol) was added dropwise to a stirred mixture of tert-butyl 3-(piperidin-4-yloxy)pyrrolidine-1-carboxylate (400 mg, 1.48 mmol) and triethylamine (0.21 mL, 1.48 mmol) in DCM (10 mL). The mixture was stirred at 20° C. for 16 h. The mixture was diluted in sat. aq. NaHCO₃(20 mL), extracted with DCM (30 mL×2). The combined organic layers were dried over anhydrous Na₂SO₄, concentrated in vacuo and purified by flash column chromatography eluting (solvent gradient: 0-50% EtOAc in petroleum ether) to afford the title compound (390 mg, 65%) as a colorless oil. LCMS (ESI) m/z: 305.2 [M-100+H]⁺.

### Step 2: benzyl 4-(pyrrolidin-3-yloxy)piperidine-1-carboxylate 2,2,2-trifluoroacetate

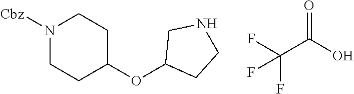

To a solution of benzyl 4-((1-(tert-butoxycarbonyl)pyrrolidin-3-yl)oxy)piperidine-1-carboxylate (390 mg, 0.96 mmol) in DCM (6 mL) was added trifluoroacetic acid (2 mL, 26.93 mmol) at 25° C. The mixture was stirred at 25° C. for 1 h. The mixture was concentrated in vacuo to afford the title compound (300 mg, 74%) as a yellow solid. LCMS (ESI) m/z: 305.2 [M+H]⁺.

### Step 3: benzyl 4-((1-sulfamoylpyrrolidin-3-yl)oxy)piperidine-1-carboxylate

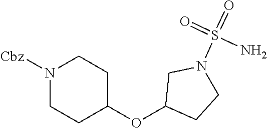 [SMILES: NS(=O)(=O)N1CCC(OC2CCN(C(=O)OCc3ccccc3)CC2)C1]

To a mixture of benzyl 4-(pyrrolidin-3-yloxy)piperidine-1-carboxylate 2,2,2-trifluoroacetate (300 mg, 0.99 mmol) in 1,4-dioxane (3.6 mL) was added sulfamide (237 mg, 2.46 mmol). The mixture was stirred at 110° C. for 16 h under nitrogen atmosphere. After cooling to room temperature, the reaction was diluted with water (20 mL) and extracted with EtOAc (20 mL×3). The combined organic lawyers were washed with brine (30 mL), dried over anhydrous Na₂SO₄, filtered and concentrated in vacuo to afford the title compound (320 mg, crude) as a yellow solid that required no further purification. LCMS (ESI) m/z: 385.1 [M+H]⁺.

### Step 4: benzyl 4-((1-(N-(5-chloro-4-(cyclopentylmethoxy)-2-fluorobenzoyl)sulfamoyl)-pyrrolidin-3-yl)oxy)piperidine-1-carboxylate

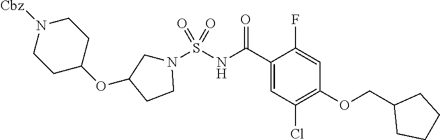 [SMILES: O=C(NS(=O)(=O)N1CCC(OC2CCN(C(=O)OCc3ccccc3)CC2)C1)c1cc(Cl)c(OCC2CCCC2)cc1F]

To a mixture of benzyl 4-(1-sulfamoylpyrrolidin-3-yl)oxypiperidine-1-carboxylate (316 mg, 0.83 mmol) and DMAP (134 mg, 1.1 mmol) in DCM (5 mL) was added 5-chloro-4-(cyclopentylmethoxy)-2-fluoro-benzoic acid (150 mg, 0.55 mmol) and EDCI (116 mg, 0.61 mmol). The resulting mixture was stirred at 20° C. for 2 h under nitrogen atmosphere. The reaction was extracted with DCM (30 mL×2). The organic layers were washed with washed with 10% citric aqueous solution (10 mL×2) and brine (20 mL), dried over anhydrous sodium sulfate, filtered and concentrated in vacuo. The residue was purified by flash column chromatography eluting (solvent gradient: 0-50% EtOAc in petroleum ether) to afford the title compound (150 mg, 43%) as a yellow solid. LCMS (ESI) m/z: 638.2 [M+H]⁺.

### Step 5: (S)-benzyl 4-((1-(N-(5-chloro-4-(cyclopentylmethoxy)-2-fluorobenzoyl)sulfamoyl)pyrrolidin-3-yl)oxy)piperidine-1-carboxylate, and (R)-benzyl 4-((1-(N-(5-chloro-4-(cyclopentylmethoxy)-2-fluorobenzoyl)sulfamoyl)pyrrolidin-3-yl)oxy)piperidine-1-carboxylate

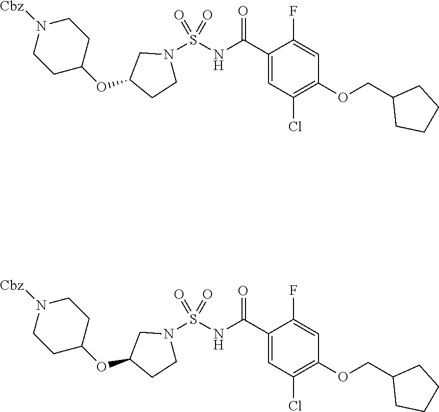 [SMILES: O=C(NS(=O)(=O)N1CC[C@H](OC2CCN(C(=O)OCc3ccccc3)CC2)C1)c1cc(Cl)c(OCC2CCCC2)cc1F ; O=C(NS(=O)(=O)N1CC[C@@H](OC2CCN(C(=O)OCc3ccccc3)CC2)C1)c1cc(Cl)c(OCC2CCCC2)cc1F]

Benzyl 4-((1-(N-(5-chloro-4-(cyclopentylmethoxy)-2-fluorobenzoyl)-sulfamoyl)pyrrolidin-3-yl)oxy)piperidine-1-carboxylate (150 mg, 0.24 mmol) was separated by using chiral SFC (DAICEL CHIRALCEL OD-H (250 mm*30 mm, 5 um); Supercritical CO₂/EtOH+0.1% NH₃·H₂O=65/35; 2.8 ml/min) to afford (S)-benzyl 4-((1-(N-(5-chloro-4-(cyclopentylmethoxy)-2-fluorobenzoyl)sulfamoyl)pyrrolidin-3-yl)oxy)piperidine-1-carboxylate (70 mg, first peak) as a white solid and (R)-benzyl 4-((1-(N-(5-chloro-4-(cyclopentylmethoxy)-2-fluorobenzoyl)sulfamoyl)pyrrolidin-3-yl)oxy)piperidine-1-carboxylate (70 mg, second peak) as a white solid. Absolute configuration was arbitrarily assigned to each enantiomer.

### Step 6: (S)-5-chloro-4-(cyclopentylmethoxy)-2-fluoro-N-((3-(piperidin-4-yloxy)-pyrrolidin-1-yl)sulfonyl)benzamide and (R)-5-chloro-4-(cyclopentylmethoxy)-2-fluoro-N-((3-(piperidin-4-yloxy)pyrrolidin-1-yl)sulfonyl)benzamide

A solution of (S)-benzyl 4-((1-(N-(5-chloro-4-(cyclopentylmethoxy)-2-fluorobenzoyl)sulfamoyl)pyrrolidin-3-yl)oxy)piperidine-1-carboxylate (35 mg, 0.05 mmol) in DCM (1.5 mL) was added PdCl₂ (9.73 mg, 0.05 mmol). After stirring at room temperature for 16 h under hydrogen atmosphere (15 psi), the reaction was filtered through Celite and concentrated in vacuo. The residue was purified by reverse phase chromatography (acetonitrile 28-58%/(0.2% formic acid) in water) to afford (S)-5-chloro-4-(cyclopentylmethoxy)-2-fluoro-N-((3-(piperidin-4-yloxy)pyrrolidin-1-yl)sulfonyl) benzamide (2 mg, 8%) as a white solid. ¹H NMR (400 MHz, DMSO-d₆) δ 8.39 (s, 1H), 7.72 (d, J=8.0 Hz, 1H), 6.93 (d, J=12.4 Hz, 1H), 4.21-4.15 (m, 1H), 3.94 (d, J=6.8 Hz, 2H), 3.64-3.57 (m, 1H), 3.45-3.35 (m, 2H), 3.27-3.11 (m, 3H), 3.02-2.78 (m, 3H), 2.34-2.26 (m, 1H), 2.03-1.85 (m, 3H), 1.81-1.66 (m, 3H), 1.65-1.47 (m, 6H), 1.41-1.29 (m, 2H). LCMS (ESI) m/z: 504.1 [M+H]⁺.

Following the same procedure, (R)-5-chloro-4-(cyclopentylmethoxy)-2-fluoro-N-((3-(piperidin-4-yloxy)pyrrolidin-1-yl)sulfonyl)benzamide was obtained as a white solid. ¹H NMR (400 MHz, DMSO-d₆) δ 7.72 (d, J=8.0 Hz, 1H), 6.93 (d, J=12.4 Hz, 1H), 4.21-4.15 (m, 1H), 3.94 (d, J=6.8 Hz, 2H), 3.65-3.57 (m, 1H), 3.45-3.35 (m, 2H), 3.27-3.10 (m, 3H), 3.03-2.78 (m, 3H), 2.34-2.28 (m, 1H), 2.02-1.87 (m, 3H), 1.79-1.68 (m, 3H), 1.63-1.50 (m, 6H), 1.39-1.31 (m, 2H). LCMS (ESI) m/z: 504.1 [M+H]⁺.

### Example 217; and Example 218: N-(((1R,5S)-1-amino-3-azabicyclo[3.1.0]hexan-3-yl)sulfonyl)-5-chloro-4-(cyclopentylmethoxy)-2-fluorobenzamide 2,2,2-trifluoroacetate, and N-(((1S,5R)-1-amino-3-azabicyclo[3.1.0]hexan-3-yl)sulfonyl)-5-chloro-4-(cyclopentyl-methoxy)-2-fluorobenzamide 2,2,2-trifluoroacetate

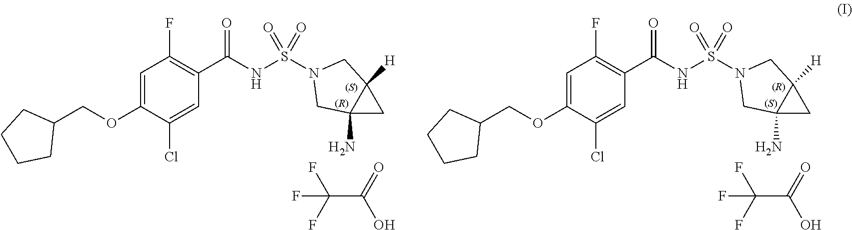

### Step 1: tert-butyl ((1R,5S)-3-(N-(5-chloro-4-(cyclopentylmethoxy)-2-fluorobenzoyl)sulfamoyl)-3-azabicyclo[3.1.0]hexan-1-yl)carbamate and tert-butyl ((1S,5R)-3-(N-(5-chloro-4-(cyclopentylmethoxy)-2-fluorobenzoyl)sulfamoyl)-3-azabicyclo[3.1.0]hexan-1-yl)carbamate

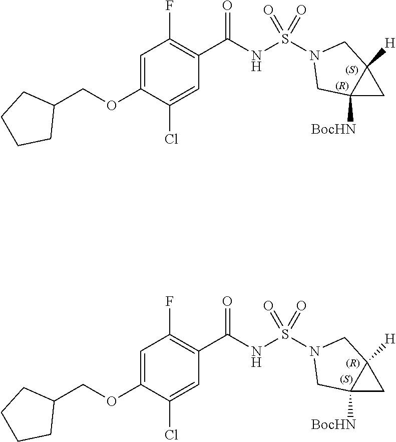

Following the procedure described in Example 31; and making non-critical variations as required to replace tert-butyl 3-(azetidin-3-yloxy)pyrrolidine-1-carboxylate with tert-butyl (3-azabicyclo[3.1.0]hexan-1-yl)carbamate, tert-butyl 3-(N-(5-chloro-4-(cyclopentylmethoxy)-2-fluorobenzoyl)sulfamoyl)-3-azabicyclo[3.1.0]hexan-1-yl)carbamate was obtained as a white solid. 3-(N-(5-chloro-4-(cyclopentylmethoxy)-2-fluorobenzoyl)sulfamoyl)-3-azabicyclo[3.1.0]hexan-1-yl)carbamate (140 mg, 0.26 mmol) was separated by using chiral SFC (DAICEL CHIRALPAK IC (250 mm*30 mm, 5 um), Supercritical CO₂/EtOH+0.1% NH₃·H₂O=70/30; 60 mL/min) to afford tert-butyl ((1R,5S)-3-(N-(5-chloro-4-(cyclopentylmethoxy)-2-fluorobenzoyl)sulfamoyl)-3-azabicyclo[3.1.0]hexan-1-yl)carbamate (50 mg, first peak) as a yellow solid and tert-butyl ((1S,5R)-3-(N-(5-chloro-4-(cyclopentylmethoxy)-2-fluorobenzoyl)sulfamoyl)-3-azabicyclo[3.1.0]hexan-1-yl)carbamate (70 mg, second peak) as a yellow solid. Absolute configuration was arbitrarily assigned to each enantiomer.

### Step 2: N-(((1R,5S)-1-amino-3-azabicyclo[3.1.0]hexan-3-yl)sulfonyl)-5-chloro-4-(cyclopentylmethoxy)-2-fluorobenzamide 2,2,2-trifluoroacetate, and N-(((1S,5R)-1-amino-3-azabicyclo[3.1.0]hexan-3-yl)sulfonyl)-5-chloro-4-(cyclopentylmethoxy)-2-fluorobenzamide 2,2,2-trifluoroacetate

Following the procedure described in Example 69 and making non-critical variations as required to replace (S)-tert-butyl 3-(4-(N-(5-chloro-4-(cyclopentylmethoxy)-2-fluorobenzoyl)sulfamoyl)phenoxy)pyrrolidine-1-carboxylate with tert-butyl ((1R,5S)-3-(N-(5-chloro-4-(cyclopentylmethoxy)-2-fluorobenzoyl)sulfamoyl)-3-azabicyclo[3.1.0]hexan-1-yl)carbamate and tert-butyl ((1S,5R)-3-(N-(5-chloro-4-(cyclopentylmethoxy)-2-fluorobenzoyl)sulfamoyl)-3-azabicyclo[3.1.0]hexan-1-yl)carbamate, the title compounds were obtained as white solids. Example 217: ¹H NMR (400 MHz, DMSO-d₆) δ 12.02 (s, 1H), 8.51 (s, 2H), 7.73 (d, J=7.6 Hz, 1H), 7.24 (d, J=12.8 Hz, 1H), 4.03 (d, J=7.2 Hz, 2H), 3.78 (d, J=9.6 Hz, 1H), 3.68-3.55 (m, 2H), 3.43 (d, J=9.6 Hz, 1H), 2.35-2.25 (m, 1H), 2.02-1.88 (m, 1H), 1.85-1.70 (m, 2H), 1.68-1.50 (m, 4H), 1.41-1.30 (m, 2H), 1.29-1.10 (m, 1H), 0.81-0.74 (m, 1H). LCMS (ESI) m/z: 432.1 [M+H]⁺. Example 218: ¹H NMR (400 MHz, DMSO-d₆) δ 12.03 (s, 1H), 8.53 (s, 2H), 7.73 (d, J=7.6 Hz, 1H), 7.24 (d, J=12.4 Hz, 1H), 4.03 (d, J=6.8 Hz, 2H), 3.78 (d, J=9.6 Hz, 1H), 3.70-3.55 (m, 2H), 3.43 (d, J=9.6 Hz, 1H), 2.40-2.25 (m, 1H), 2.02-1.93 (m, 1H), 1.85-1.69 (m, 2H), 1.68-1.46 (m, 4H), 1.42-1.30 (m, 2H), 1.28-1.19 (m, 1H), 0.80-0.70 (m, 1H). LCMS (ESI) m/z: 432.2 [M+H]⁺.

### Example 219: (S)—N-((3-aminoazepan-1-yl)sulfonyl)-5-chloro-4-(cyclopentylmethoxy)-2-fluorobenzamide 2,2,2-trifluoroacetate

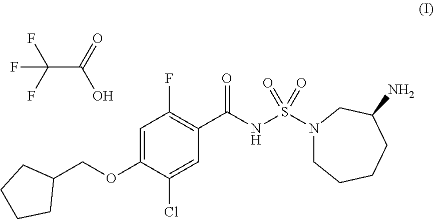 [SMILES: N[C@H]1CCCCN(S(=O)(=O)NC(=O)c2cc(Cl)c(OCC3CCCC3)cc2F)C1 ; O=C(O)C(F)(F)F]

### Step 1: tert-butyl (1-(N-(5-chloro-4-(cyclopentylmethoxy)-2-fluorobenzoyl)sulfamoyl) azepan-3-yl)carbamate

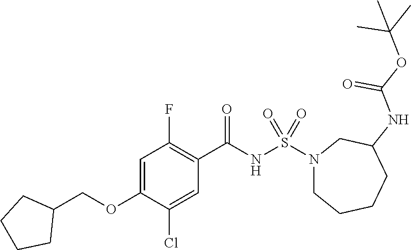 [SMILES: CC(C)(C)OC(=O)NC1CCCCN(S(=O)(=O)NC(=O)c2cc(Cl)c(OCC3CCCC3)cc2F)C1]

Following the procedure described in Example 31; and making non-critical variations as required to replace tert-butyl 3-(azetidin-3-yloxy)pyrrolidine-1-carboxylate with tert-butyl azepan-3-ylcarbamate, the title compound was obtained as a white solid.

### Step 2: tert-butyl (S)-(1-(N-(5-chloro-4-(cyclopentylmethoxy)-2-fluorobenzoyl)-sulfamoyl)azepan-3-yl)carbamate, and tert-butyl (R)-(1-(N-(5-chloro-4-(cyclopentylmethoxy)-2-fluorobenzoyl)sulfamoyl)azepan-3-yl)carbamate

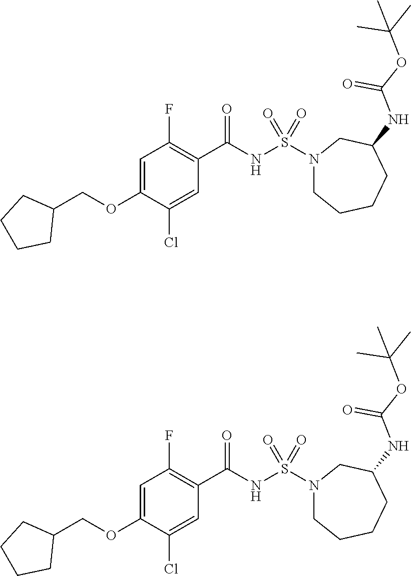 [SMILES: CC(C)(C)OC(=O)N[C@H]1CCCCN(S(=O)(=O)NC(=O)c2cc(Cl)c(OCC3CCCC3)cc2F)C1 ; CC(C)(C)OC(=O)N[C@@H]1CCCCN(S(=O)(=O)NC(=O)c2cc(Cl)c(OCC3CCCC3)cc2F)C1]

tert-butyl (1-(N-(5-chloro-4-(cyclopentylmethoxy)-2-fluorobenzoyl)sulfamoyl) azepan-3-yl)carbamate (140 mg, 0.26 mmol) was separated by using chiral SFC (DAICEL CHIRALPAK IC (250 mm*30 mm, 5 um), Supercritical CO₂/EtOH+0.1% NH₃·H₂O=70/30; 60 mL/min) to afford tert-butyl ((1R,5S)-3-(N-(5-chloro-4-(cyclopentylmethoxy)-2-fluorobenzoyl)sulfamoyl)-3-azabicyclo[3.1.0]hexan-1-yl)carbamate (50 mg, first peak) as a yellow solid and tert-butyl ((1S,5R)-3-(N-(5-chloro-4-(cyclopentylmethoxy)-2-fluorobenzoyl)sulfamoyl)-3-azabicyclo[3.1.0]hexan-1-yl)carbamate (70 mg, second peak) as a yellow solid. Absolute configuration was arbitrarily assigned to each enantiomer.

### Step 3: (S)—N-((3-aminoazepan-1-yl)sulfonyl)-5-chloro-4-(cyclopentylmethoxy)-2-fluorobenzamide 2,2,2-trifluoroacetate

Following the procedure described in Example 69 and making non-critical variations as required to replace (S)-tert-butyl 3-(4-(N-(5-chloro-4-(cyclopentylmethoxy)-2-fluorobenzoyl)sulfamoyl)phenoxy)pyrrolidine-1-carboxylate with tert-butyl (S)-(1-(N-(5-chloro-4-(cyclopentylmethoxy)-2-fluorobenzoyl)sulfamoyl)azepan-3-yl)carbamate, the title compound was obtained as a white solid. ¹H NMR (400 MHz, DMSO-d₆) δ 8.04 (s, 2H), 7.80 (d, J=8.0 Hz, 1H), 7.01 (d, J=12.4 Hz, 1H), 3.96 (d, J=6.8 Hz, 2H), 3.90-3.80 (m, 1H), 3.33-3.18 (m, 3H), 3.12-3.00 (m, 1H), 2.93-2.80 (m, 1H), 2.38-2.25 (m, 1H), 1.84-1.70 (m, 4H), 1.70-1.60 (m, 3H), 1.59-1.49 (m, 3H), 1.49-1.41 (m, 2H), 1.40-1.26 (m, 2H). LCMS (ESI) m/z: 448.4 [M+H]⁺.

### Example 220 and Example 221: 5-chloro-4-(cyclopentylmethoxy)-2-fluoro-N-(((3aS,6aR)-hexahydrocyclopenta[b]pyrrol-1(2H)-yl)sulfonyl)benzamide, and 5-chloro-4-(cyclopentylmethoxy)-2-fluoro-N-(((3aR,6aS)-hexahydrocyclopenta[b]pyrrol-1(2H)-yl)sulfonyl)benzamide

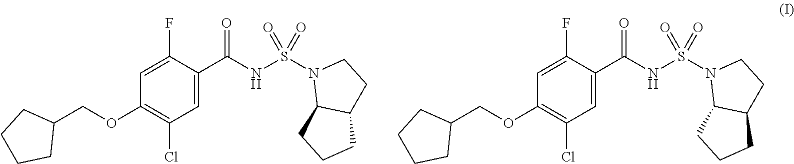

### Step 1: 5-chloro-4-(cyclopentylmethoxy)-2-fluoro-N-((hexahydrocyclopenta[b]pyrrol-1(2H)-yl)sulfonyl)benzamide

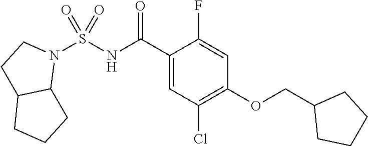 [SMILES: O=C(NS(=O)(=O)N1CCC2CCCC21)c1cc(Cl)c(OCC2CCCC2)cc1F]

Following the procedure described in Example 31; and making non-critical variations as required to replace tert-butyl 3-(azetidin-3-yloxy)pyrrolidine-1-carboxylate with octahydrocyclopenta[b]pyrrole, the title compound was obtained as a white solid. LCMS (ESI) m/z: 445.0 [M+H]⁺.

### Step 2: 5-chloro-4-(cyclopentylmethoxy)-2-fluoro-N-(((3aS,6aR)-hexahydrocyclo-penta[b]pyrrol-1(2H)-yl)sulfonyl)benzamide, and 5-chloro-4-(cyclopentylmethoxy)-2-fluoro-N-(((3aR,6aS)-hexahydrocyclopenta[b]pyrrol-1(2H)-yl)sulfonyl)benzamide

5-chloro-4-(cyclopentylmethoxy)-2-fluoro-N-((hexahydrocyclopenta[b]pyrrol-1(2H)-yl)sulfonyl)benzamide (146 mg, 0.33 mmol) was separated by using chiral SFC (DAICEL CHIRALCEL OD-H (250 mm*30 mm, 5 um), Supercritical CO₂/EtOH+0.1% NH₃·H₂O=50/50; 80 mL/min) to afford 5-chloro-4-(cyclopentylmethoxy)-2-fluoro-N-(((3aR,6aS)-hexahydrocyclopenta[b]pyrrol-1(2H)-yl)sulfonyl)benzamide (25 mg, first peak) as a white solid and 5-chloro-4-(cyclopentylmethoxy)-2-fluoro-N-(((3aS,6aR)-hexahydrocyclopenta[b]pyrrol-1(2H)-yl)sulfonyl)benzamide (68 mg, second peak) as a white solid. Absolute configuration was arbitrarily assigned to each enantiomer. Example 220: ¹H NMR (400 MHz, DMSO-d₆) δ 11.79 (s, 1H), 7.70 (d, J=7.6 Hz, 1H), 7.22 (d, J=12.4 Hz, 1H), 4.33-4.26 (m, 1H), 4.02 (d, J=6.8 Hz, 2H), 3.63-3.53 (m, 1H), 3.39-3.30 (m, 1H), 2.74-2.67 (m, 1H), 2.35-2.29 (m, 1H), 1.89-1.51 (m, 13H), 1.42-1.31 (m, 3H). LCMS (ESI) m/z: 445.0 [M+H]⁺. Example 221: ¹H NMR (400 MHz, DMSO-d₆) δ 11.78 (s, 1H), 7.71 (d, J=7.6 Hz, 1H), 7.22 (d, J=12.4 Hz, 1H), 4.32-4.25 (m, 1H), 4.02 (d, J=6.8 Hz, 2H), 3.62-3.54 (m, 1H), 3.40-3.34 (m, 1H), 2.73-2.66 (m, 1H), 2.37-2.28 (m, 1H), 1.86-1.52 (m, 13H), 1.42-1.28 (m, 3H). LCMS (ESI) m/z: 445.0 [M+H]⁺.

### Example 222 and Example 223: (R)-5-chloro-4-(cyclopentylmethoxy)-2-fluoro-N-((2-methylpyrrolidin-1-yl)sulfonyl)benzamide, and (S)-5-chloro-4-(cyclopentylmethoxy)-2-fluoro-N-((2-methylpyrrolidin-1-yl)sulfonyl)-benzamide

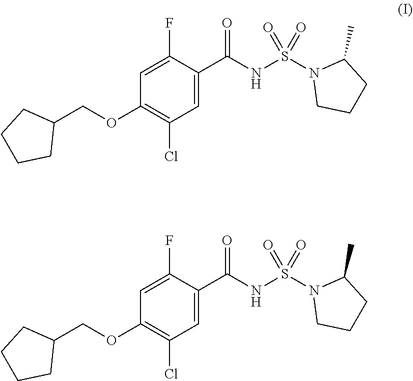 [SMILES: C[C@@H]1CCCN1S(=O)(=O)NC(=O)c1cc(Cl)c(OCC2CCCC2)cc1F ; C[C@H]1CCCN1S(=O)(=O)NC(=O)c1cc(Cl)c(OCC2CCCC2)cc1F]

### Step 1: 5-chloro-4-(cyclopentylmethoxy)-2-fluoro-N-((2-methylpyrrolidin-1-yl)sulfonyl)benzamide

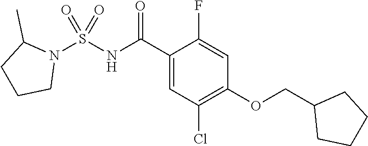 [SMILES: CC1CCCN1S(=O)(=O)NC(=O)c1cc(Cl)c(OCC2CCCC2)cc1F]

Following the procedure described in Example 31; and making non-critical variations as required to replace tert-butyl 3-(azetidin-3-yloxy)pyrrolidine-1-carboxylate with 2-methylpyrrolidine, the title compound was obtained as a white solid. LCMS (ESI) m/z: 419.2 [M+H]⁺.

### Step 2: (R)-5-chloro-4-(cyclopentylmethoxy)-2-fluoro-N-((2-methylpyrrolidin-1-yl)-sulfonyl)benzamide, and (S)-5-chloro-4-(cyclopentylmethoxy)-2-fluoro-N-((2-methylpyrrolidin-1-yl)sulfonyl)-benzamide

5-chloro-4-(cyclopentylmethoxy)-2-fluoro-N-((2-methylpyrrolidin-1-yl)sulfonyl)benzamide (50 mg, 0.12 mmol) was separated by using chiral SFC (DAICEL CHIRALPAK IC (250 mm*30 mm, 5 um), Supercritical CO₂/EtOH+0.1% NH₃·H₂O=75/25; 60 mL/min) to afford (R)-5-chloro-4-(cyclopentylmethoxy)-2-fluoro-N-((2-methylpyrrolidin-1-yl)sulfonyl)benzamide (6 mg, first peak) as a white solid and (S)-5-chloro-4-(cyclopentylmethoxy)-2-fluoro-N-((2-methylpyrrolidin-1-yl)sulfonyl)benzamide (6 mg, second peak) as a white solid. Absolute configuration was arbitrarily assigned to each enantiomer. Example 222:: ¹H NMR (400 MHz, DMSO-d₆) δ 11.75 (s, 1H), 7.71 (d, J=7.2 Hz, 1H), 7.22 (d, J=12.4 Hz, 1H), 4.18-4.07 (m, 1H), 4.02 (d, J=6.8 Hz, 2H), 3.54-3.44 (m, 1H), 3.40-3.35 (m, 1H), 2.38-2.28 (m, 1H), 2.01-1.84 (m, 2H), 1.83-1.70 (m, 3H), 1.67-1.45 (m, 5H), 1.42-1.28 (m, 2H), 1.19 (d, J=6.4 Hz, 3H). LCMS (ESI) m/z: 441.0 [M+Na]⁺. Example 223:: ¹H NMR (400 MHz, DMSO-d₆) δ 11.76 (s, 1H), 7.71 (d, J=7.6 Hz, 1H), 7.23 (d, J=12.4 Hz, 1H), 4.17-4.07 (m, 1H), 4.02 (d, J=6.8 Hz, 2H), 3.54-3.45 (m, 1H), 3.40-3.35 (m, 1H), 2.40-2.29 (m, 1H), 2.02-1.84 (m, 2H), 1.83-1.71 (m, 3H), 1.65-1.50 (m, 5H), 1.39-1.30 (m, 2H), 1.19 (d, J=6.4 Hz, 3H). LCMS (ESI) m/z: 441.0 [M+Na]⁺.

### Example 224; and Example 225: (S)-5-chloro-4-(cyclopentylmethoxy)-2-fluoro-N-((2-methylpiperidin-1-yl)sulfonyl)benzamide, and (R)-5-chloro-4-(cyclopentylmethoxy)-2-fluoro-N-((2-methylpiperidin-1-yl)sulfonyl)-benzamide

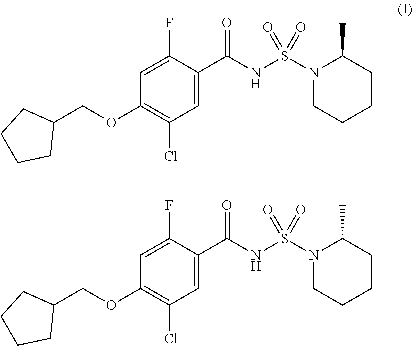 [SMILES: C[C@H]1CCCCN1S(=O)(=O)NC(=O)c1cc(Cl)c(OCC2CCCC2)cc1F ; C[C@@H]1CCCCN1S(=O)(=O)NC(=O)c1cc(Cl)c(OCC2CCCC2)cc1F]

### Step 1: 5-chloro-4-(cyclopentylmethoxy)-2-fluoro-N-((2-methylpiperidin-1-yl)sulfonyl)benzamide

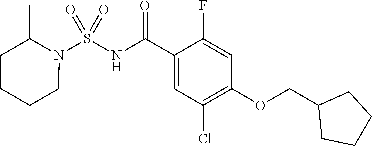 [SMILES: CC1CCCCN1S(=O)(=O)NC(=O)c1cc(Cl)c(OCC2CCCC2)cc1F]

Following the procedure described in Example 31; and making non-critical variations as required to replace tert-butyl 3-(azetidin-3-yloxy)pyrrolidine-1-carboxylate with 2-methylpiperidine, the title compound was obtained as a white solid. LCMS (ESI) m/z: 433.2 [M+H]⁺.

### Step 2: (S)-5-chloro-4-(cyclopentylmethoxy)-2-fluoro-N-((2-methylpiperidin-1-yl)sulfonyl)benzamide, and (R)-5-chloro-4-(cyclopentylmethoxy)-2-fluoro-N-((2-methylpiperidin-1-yl)sulfonyl)-benzamide

5-chloro-4-(cyclopentylmethoxy)-2-fluoro-N-((2-methylpiperidin-1-yl)sulfonyl)benzamide (190 mg, 0.44 mmol) was separated by using chiral SFC (DAICEL CHIRALPCEL OD-H (250 mm*30 mm, 5 um), Supercritical CO₂/EtOH+0.1% NH₃·H₂O=50/50; 80 mL/min) to afford (S)-5-chloro-4-(cyclopentylmethoxy)-2-fluoro-N-((2-methylpiperidin-1-yl)sulfonyl)benzamide (58 mg, first peak) as a white solid and (R)-5-chloro-4-(cyclopentylmethoxy)-2-fluoro-N-((2-methylpiperidin-1-yl)sulfonyl)benzamide (47 mg, second peak) as a white solid. Absolute configuration was arbitrarily assigned to each enantiomer. Example 224:: ¹H NMR (400 MHz, DMSO-d₆) δ 11.81 (s, 1H), 7.66 (d, J=7.6 Hz, 1H), 7.23 (d, J=12.4 Hz, 1H), 4.18-4.08 (m, 1H), 4.02 (d, J=6.8 Hz, 2H), 3.72-3.63 (m, 1H), 3.13 (t, J=11.2 Hz, 1H), 2.40-2.28 (m, 1H), 1.80-1.76 (m, 2H), 1.68-1.38 (m, 9H), 1.35-1.28 (m, 3H), 1.18 (d, J=6.8 Hz, 3H). LCMS (ESI) m/z: 433.0 [M+H]⁺. Example 225:: ¹H NMR (400 MHz, DMSO-d₆) δ 11.81 (s, 1H), 7.66 (d, J=7.6 Hz, 1H), 7.23 (d, J=12.4 Hz, 1H), 4.18-4.06 (m, 1H), 4.02 (d, J=6.8 Hz, 2H), 3.75-3.60 (m, 1H), 3.14 (t, J=11.2 Hz, 1H), 2.36-2.25 (m, 1H), 1.80-1.76 (m, 2H), 1.66-1.40 (m, 9H), 1.38-1.28 (m, 3H), 1.18 (d, J=6.8 Hz, 3H). LCMS (ESI) m/z: 433.0 [M+H]⁺.

### Example 226: 5-chloro-4-(cyclopentylmethoxy)-2-fluoro-N-((3-(methylamino)azetidin-1-yl)sulfonyl)benzamide formate

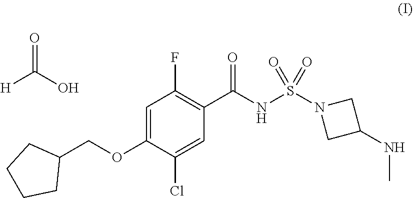 [SMILES: O=CO ; CNC1CN(S(=O)(=O)NC(=O)c2cc(Cl)c(OCC3CCCC3)cc2F)C1]

Following the procedure described in Example 31; and making non-critical variations as required to replace tert-butyl 3-(azetidin-3-yloxy)pyrrolidine-1-carboxylate with tert-butyl azetidin-3-yl(methyl)carbamate, the title compound was obtained as a white solid. ¹H NMR (400 MHz, DMSO-d₆) δ 8.13 (s, 1H), 7.77 (d, J=8.0 Hz, 1H), 6.97 (d, J=12.4 Hz, 1H), 4.12-3.97 (m, 2H), 3.96 (d, J=6.8 Hz, 2H), 3.84-3.76 (m, 1H), 3.75-3.69 (m, 2H), 2.52 (s, 3H), 2.37-2.26 (m, 1H), 1.85-1.71 (m, 2H), 1.66-1.45 (m, 4H), 1.43-1.25 (m, 2H). LCMS (ESI) m/z: 420.1 [M+H]⁺.

### Example 227: N-(azepan-1-ylsulfonyl)-5-chloro-4-(cyclopentylmethoxy)-2-fluorobenzamide

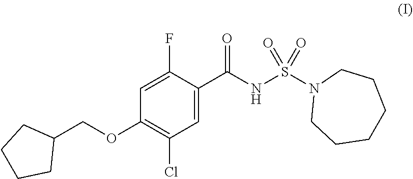 [SMILES: O=C(NS(=O)(=O)N1CCCCCC1)c1cc(Cl)c(OCC2CCCC2)cc1F]

Following the procedure described in Example 31; and making non-critical variations as required to replace tert-butyl 3-(azetidin-3-yloxy)pyrrolidine-1-carboxylate with azepane, the title compound was obtained as a white solid. ¹H NMR (400 MHz, DMSO-d₆) δ 11.76 (s, 1H), 7.70 (d, J=7.6 Hz, 1H), 7.22 (d, J=12.4 Hz, 1H), 4.02 (d, J=6.8 Hz, 2H), 3.41-3.32 (m, 4H), 2.34-2.32 (m, 1H), 1.83-1.73 (m, 2H), 1.70-1.60 (m, 6H), 1.57-1.50 (m, 6H), 1.39-1.30 (m, 2H). LCMS (ESI) m/z: 433.0 [M+H]⁺.

### Example 228: N-((1-oxa-6-azaspiro[3.3]heptan-6-yl)sulfonyl)-5-chloro-4-(cyclopentylmethoxy)-2-fluorobenzamide

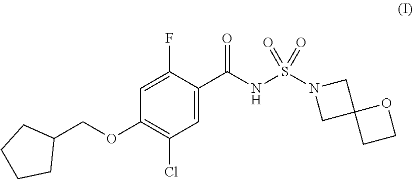 [SMILES: O=C(NS(=O)(=O)N1CC2(CCO2)C1)c1cc(Cl)c(OCC2CCCC2)cc1F]

Following the procedure described in Example 31 and making non-critical variations as required to replace tert-butyl 3-(azetidin-3-yloxy)pyrrolidine-1-carboxylate with 1-oxa-6-azaspiro[3.3]heptane, the title compound was obtained as a white solid. ¹H NMR (400 MHz, DMSO-d₆) δ 7.74 (d, J=8.0 Hz, 1H), 7.07 (s, 1H), 6.97 (d, J=12.0 Hz, 1H), 4.36 (t, J=7.6 Hz, 2H), 3.96 (d, J=6.8 Hz, 2H), 3.85 (s, 4H), 2.76 (t, J=7.2 Hz, 2H), 2.37-2.26 (m, 1H), 1.84-1.70 (m, 2H), 1.67-1.47 (m, 4H), 1.42-1.31 (m, 2H). LCMS (ESI) m/z: 433.0 [M+H]⁺.

### Example 229: N-((4-azaspiro[2.4]heptan-4-yl)sulfonyl)-5-chloro-4-(cyclopentylmethoxy)-2-fluorobenzamide

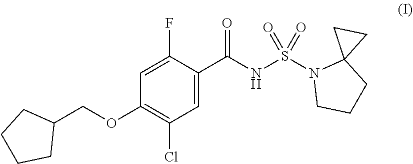 [SMILES: O=C(NS(=O)(=O)N1CCCC12CC2)c1cc(Cl)c(OCC2CCCC2)cc1F]

### Step 1: 4-azaspiro[2.4]heptane-4-sulfonamide

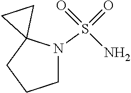 [SMILES: NS(=O)(=O)N1CCCC12CC2]

To a mixture of 4-azaspiro[2.4]heptane oxalate oxalic acid (100 mg, 0.53 mmol) in 1,4-dioxane (5 mL) was added sulfamide (130 mg, 1.34 mmol) and NEt₃ (162 mg, 1.60 mmol). The mixture was stirred at 110° C. for 16 h under nitrogen atmosphere. After cooling to room temperature, the reaction was diluted with water (50 mL) and extracted with EtOAc (50 mL×2). The combined organic lawyers were dried over anhydrous Na₂SO₄, filtered and concentrated in vacuo to afford the title compound (90 mg, crude) as a yellow oil that required no further purification.

### Step 2: N-((4-azaspiro[2.4]heptan-4-yl)sulfonyl)-5-chloro-4-(cyclopentylmethoxy)-2-fluorobenzamide

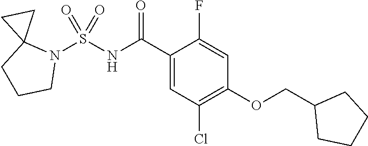 [SMILES: O=C(NS(=O)(=O)N1CCCC12CC2)c1cc(Cl)c(OCC2CCCC2)cc1F]

Following the procedure described in Example 31 and making non-critical variations as required to replace tert-butyl 3-((1-sulfamoylazetidin-3-yl)oxy)pyrrolidine-1-carboxylate with 4-azaspiro[2.4]heptane-4-sulfonamide, the title compound was obtained as a white solid. ¹H NMR (400 MHz, DMSO-d₆) δ 11.90 (s, 1H), 7.67 (d, J=7.2 Hz, 1H), 7.24 (d, J=12.0 Hz, 1H), 4.03 (d, J=6.8 Hz, 2H), 3.84 (t, J=7.2 Hz, 2H), 2.38-2.28 (m, 1H), 1.95-1.85 (m, 2H), 1.83-1.73 (m, 2H), 1.72-1.67 (m, 2H), 1.66-1.50 (m, 4H), 1.41-1.29 (m, 2H), 1.25-1.18 (m, 2H), 0.65-0.59 (m, 2H). LCMS (ESI) m/z: 431.1 [M+H]⁺.

### Example 230: N-(((1R,5S)-3-azabicyclo[3.2.1]octan-3-yl)sulfonyl)-5-chloro-4-(cyclopentylmethoxy)-2-fluorobenzamide

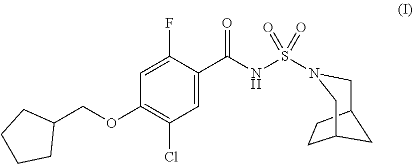 [SMILES: O=C(NS(=O)(=O)N1CC2CCC(C2)C1)c1cc(Cl)c(OCC2CCCC2)cc1F]

Following the procedure described in Example 31 and making non-critical variations as required to replace tert-butyl 3-(azetidin-3-yloxy)pyrrolidine-1-carboxylate with 4-azaspiro[2.4]heptane-4-sulfonamide, the title compound was obtained as a white solid. ¹H NMR (400 MHz, DMSO-d₆) δ 11.74 (s, 1H), 7.69 (d, J=7.6 Hz, 1H), 7.22 (d, J=12.4 Hz, 1H), 4.02 (d, J=6.8 Hz, 2H), 3.47-3.43 (m, 2H), 3.01 (d, J=11.2 Hz, 2H), 2.38-2.31 (m, 1H), 2.30-2.22 (m, 2H), 1.81-1.73 (m, 2H), 1.63-1.45 (m, 1OH), 1.40-1.33 (m, 2H). LCMS (ESI) m/z: 445.2 [M+H]⁺.

### Example 231: N-((1S,6R)-3,8-diazabicyclo[4.2.0]octan-3-ylsulfonyl)-5-chloro-4-(cyclopentylmethoxy)-2-fluorobenzamide 2,2,2-trifluoroacetate

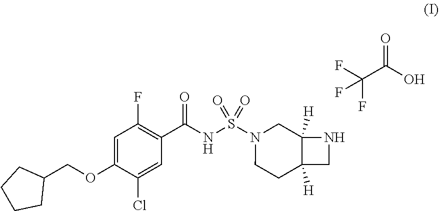

Following the procedure described in Example 31 and making non-critical variations as required to replace tert-butyl 3-(azetidin-3-yloxy)pyrrolidine-1-carboxylate with tert-butyl (1S,6R)-3,8-diazabicyclo[4.2.0]octane-8-carboxylate, the title compound was obtained as a white solid. ¹H NMR (400 MHz, DMSO-d₆) δ 12.00 (s, 1H), 8.90 (s, 1H), 7.74 (d, J=7.2 Hz, 1H), 7.22 (d, J=12.4 Hz, 1H), 4.64-4.52 (m, 1H), 4.09-3.93 (m, 3H), 3.80-3.73 (m, 1H), 3.68-3.52 (m, 4H), 3.31-3.19 (m, 1H), 2.90-2.83 (m, 1H), 2.38-2.28 (m, 1H), 2.05-1.96 (m, 1H), 1.86-1.71 (m, 3H), 1.67-1.48 (m, 4H), 1.41-1.29 (m, 2H). LCMS (ESI) m/z: 446.1 [M+H]⁺.

### Example 232: (S)—N-((3-(aminomethyl)pyrrolidin-1-yl)sulfonyl)-5-chloro-4-(cyclopentylmethoxy)-2-fluorobenzamide 2,2,2-trifluoroacetate

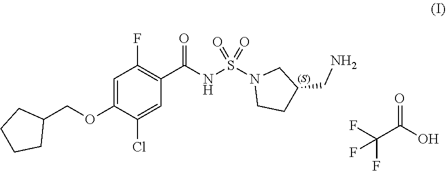

Following the procedure described in Example 31 and making non-critical variations as required to replace tert-butyl 3-(azetidin-3-yloxy)pyrrolidine-1-carboxylate with tert-butyl (R)-(pyrrolidin-3-ylmethyl)carbamate, the title compound was obtained as a white solid. ¹H NMR (400 MHz, DMSO-d₆) δ 11.87 (s, 1H), 7.79 (s, 2H), 7.73 (d, J=7.2 Hz, 1H), 7.24 (d, J=12.0 Hz, 1H), 4.03 (d, J=6.8 Hz, 2H), 3.75-3.65 (m, 1H), 3.55-3.45 (m, 1H), 3.44-3.35 (m, 1H), 3.21-3.13 (m, 1H), 2.87 (s, 2H), 2.45-2.40 (m, 1H), 2.40-2.28 (m, 1H), 2.10-2.00 (m, 1H), 1.84-1.72 (m, 2H), 1.72-1.50 (m, 5H), 1.40-1.29 (m, 2H). LCMS (ESI) m/z: 434.0 [M+H]⁺.

### Example 233: N-((3-aminoazetidin-1-yl)sulfonyl)-5-chloro-4-(cyclopentylmethoxy)-2-fluorobenzamide

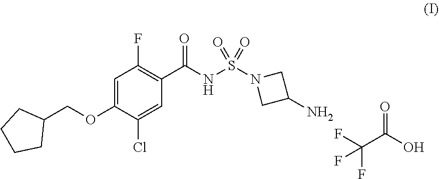

Following the procedure described in Example 31 and making non-critical variations as required to replace tert-butyl 3-((1-sulfamoylazetidin-3-yl)oxy)pyrrolidine-1-carboxylate with tert-butyl (1-sulfamoylazetidin-3-yl)carbamate (reference: WO200624823), the title compound was obtained as a white solid. ¹H NMR (400 MHz, DMSO-d₆) δ 8.14 (s, 2H), 7.77 (d, J=8.0 Hz, 1H), 6.98 (d, J=12.4 Hz, 1H), 4.08-3.94 (m, 4H), 3.86-3.80 (m, 1H), 3.70-3.62 (m, 1H), 2.32-2.24 (m, 1H), 1.83-1.72 (m, 2H), 1.68-1.49 (m, 4H), 1.42-1.27 (m, 2H). LCMS (ESI) m/z: 406.1 [M+H]⁺.

### Example 234: 5-chloro-4-(cyclopentylmethoxy)-N-((3-(dimethylamino)azetidin-1-yl)sulfonyl)-2-fluorobenzamide

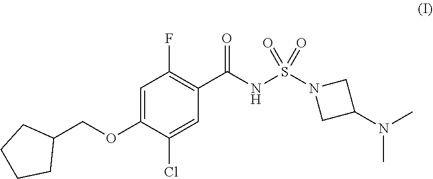 [SMILES: CN(C)C1CN(S(=O)(=O)NC(=O)c2cc(Cl)c(OCC3CCCC3)cc2F)C1]

To a mixture of N-((4-(azetidin-3-yloxy)piperidin-1-yl)sulfonyl)-5-chloro-4-(cyclopentylmethoxy)-2-fluorobenzamide (200 mg, 0.49 mmol, Example 233: in DCM (10 mL) was added formaldehyde (0.37 mL, 4.93 mmol, 37% in water) and NaBH(OAc)₃ (522 mg, 2.46 mmol). The resulting mixture was stirred at room temperature for 16 h. The mixture was quenched with saturated aqueous NaHCO₃ solution (20 mL) to pH>7 and then extracted with EtOAc (50 mL×2). The combined organic lawyers were washed with brine (60 mL), then dried over anhydrous Na₂SO₄, filtered and concentrated in vacuo. The crude residue was purified by reverse phase chromatography (solvent gradient: acetonitrile 22-52%/(0.2% fomic acid) in water) to afford the title compound (28 mg, 13%) as a white solid. ¹H NMR (400 MHz, DMSO-d) 6.7.77 (d, J=8.0 Hz, 1H), 7.05 (d, J=12.4 Hz, 1H), 4.03-3.92 (m, 4H), 3.88-3.78 (m, 2H), 3.62-3.55 (m, 1H), 2.51 (s, 6H), 2.36-2.25 (m, 1H), 1.79-1.73 (m, 2H), 1.70-1.46 (m, 4H), 1.40-1.30 (m, 2H). LCMS (ESI) m/z: 434.2 [M+H]⁺.

### Example 236: 4-(benzyloxy)-5-cyclopropyl-2-fluoro-N-((4-((1-methylazetidin-3-yl)oxy)piperidin-1-yl)sulfonyl)benzamide

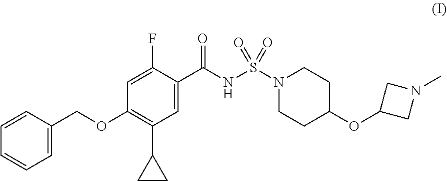 [SMILES: CN1CC(OC2CCN(S(=O)(=O)NC(=O)c3cc(C4CC4)c(OCc4ccccc4)cc3F)CC2)C1]

### Step 1: tert-butyl 4-(benzyloxy)-5-chloro-2-fluorobenzoate

To a solution of tert-butyl 5-chloro-2,4-difluoro-benzoate (5.0 g, 20.11 mmol) and Cs₂CO₃ (13.1 g, 40.22 mmol) in DMSO (50 mL), benzyl alcohol (2.17 g, 20.11 mmol) was added. The reaction was stirred at 80° C. under nitrogen atmosphere for 16 h. After cooling to room temperature, the reaction was diluted with water (100 mL) and extracted with EtOAc (100 mL×2). The combined organic lawyers were washed with brine (100 mL×3), dried over anhydrous Na₂SO₄, filtered and concentrated in vacuo. The crude residue was purified by silica gel chromatography (solvent gradient: 0-1% EtOAc in petroleum ether) to afford the title compound (4.7 g, 69%) as colorless oil. LCMS (ESI) m/z: 281.1 [M-56+H]⁺.

### Step 2: tert-butyl 4-(benzyloxy)-5-cyclopropyl-2-fluorobenzoate

To a solution of tert-butyl 4-benzyloxy-5-chloro-2-fluoro-benzoate (2.5 g, 7.42 mmol), K₃PO₄ (4.73 g, 22.27 mmol) and cyclopropylboronicacid (956 mg, 11.13 mmol) in toluene (17.5 mL) and water (2.5 mL), Pd(OAc)₂ (166 mg, 0.74 mmol) and 2-dicyclohexylphosphino-2′,6′-dimethoxybiphenyl (304 mg, 0.74 mmol) was added under nitrogen atmosphere at room temperature. The reaction was stirred at 100° C. for 16 h. After cooling to room temperature, the reaction was diluted with water (50 mL) and extracted with EtOAc (50 mL×3). The combined organic lawyers were washed with brine (50 mL), dried over anhydrous Na₂SO₄, filtered and concentrated in vacuo. The crude residue was purified by silica gel chromatography (solvent gradient: 0-2% EtOAc in petroleum ether) to afford the title compound (2.1 g, 82%) as colorless oil. LCMS (ESI) m/z: 287.1 [M-56+H]⁺.

### Step 3: 4-(benzyloxy)-5-cyclopropyl-2-fluorobenzoic acid

To a solution of tert-butyl 4-(benzyloxy)-5-cyclopropyl-2-fluorobenzoate (200 mg, 0.58 mmol) in DCM (2 mL) was added TFA (2 mL) under nitrogen atmosphere at room temperature. The reaction was stirred at room temperature for 2 h. The mixture was concentrated in vacuo to afford the title compound (160 mg, crude) as a white solid that required no further purification. LCMS (ESI) m/z: 287.2 [M-56+H]⁺.

### Step 4: 4-(benzyloxy)-5-cyclopropyl-2-fluoro-N-((4-((1-methylazetidin-3-yl)oxy)piperidin-1-yl)sulfonyl)benzamide

Following the procedure described in Example 38 and making non-critical variations as required to replace tert-butyl 3-(piperidin-4-yloxy)pyrrolidine-1-carboxylate with tert-butyl 3-(piperidin-4-yloxy)azetidine-1-carboxylate (reference: Tetrahedron Lett., 2007, 48, 791-794), 4-(benzyloxy)-5-chloro-2-fluorobenzoic acid with 4-(benzyloxy)-5-cyclopropyl-2-fluorobenzoic acid, the title compound was obtained as a white solid. ¹H NMR (400 MHz, DMSO-d₆) δ 8.24 (s, 1H), 7.50-7.43 (m, 2H), 7.42-7.35 (m, 2H), 7.33-7.28 (m, 1H), 7.22 (d, J=8.8 Hz, 1H), 6.79 (d, J=12.8 Hz, 1H), 5.17 (s, 2H), 4.23-4.14 (m, 1H), 3.80-3.73 (m, 2H), 3.37-3.28 (m, 3H), 3.13-3.02 (m, 2H), 2.77-2.68 (m, 2H), 2.41 (s, 3H), 2.07-1.98 (m, 1H), 1.80-1.72 (m, 2H), 1.46-1.35 (m, 2H), 0.93-0.82 (m, 2H), 0.62-0.49 (m, 2H). LCMS (ESI) m/z: 518.3 [M+H]⁺.

### Example 237: 5-cyclopropyl-2-fluoro-4-isobutoxy-N-((4-((1-methylazetidin-3-yl)oxy)piperidin-1-yl)sulfonyl)benzamide formic acid

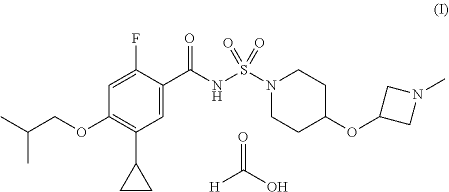

### Step 1: tert-butyl 5-cyclopropyl-2-fluoro-4-hydroxybenzoate

To a solution of tert-butyl 4-(benzyloxy)-5-cyclopropyl-2-fluorobenzoate (1.4 g, 4.09 mmol) in ethanol (30 mL) and Pd/C (870 mg, 0.82 mmol) was added at room temperature. The mixture was stirred at room temperature under hydrogen atmosphere (15 psi) for 16 h. The reaction was filtered and concentrated in vacuo to afford the title compound (1.0 g, crude) as colorless oil that required no further purification. LCMS (ESI) m/z: 197.1 [M-56+H].

### Step 2: tert-butyl 5-cyclopropyl-2-fluoro-4-isobutoxybenzoate

To a stirred solution of tert-butyl 5-cyclopropyl-2-fluoro-4-hydroxybenzoate (250 mg, 0.10 mmol) in DMF (5 mL) was added K₂CO₃ (1.1 g, 7.9 mmol) and 1-iodo-2-methylpropane (912 mg, 4.95 mmol) at room temperature under nitrogen atmosphere. Then the reaction was stirred at 70° C. for 16 h. After cooling to room temperature, the reaction was diluted with water (50 mL) and extracted with EtOAc (50 mL×3). The combined organic layers were washed with brine (100 mL×4), dried over anhydrous Na₂SO₄, filtered and concentrated in vacuo to afford the title compound (300 mg, 98%) as yellow oil that required no further purification. LCMS (ESI) m/z: 253.1 [M-56+H]⁺.

### Step 3: 5-cyclopropyl-2-fluoro-4-isobutoxy-N-((4-((1-methylazetidin-3-yl)oxy)piperidin-1-yl)sulfonyl)benzamide formic acid

Following the procedure described in Example 236 and making non-critical variations as required to replace tert-butyl 4-(benzyloxy)-5-cyclopropyl-2-fluorobenzoate with tert-butyl 5-cyclopropyl-2-fluoro-4-isobutoxybenzoate, tert-butyl 4-(benzyloxy)-5-cyclopropyl-2-fluorobenzoate with tert-butyl 5-cyclopropyl-2-fluoro-4-isobutoxybenzoate, the title compound was obtained as a white solid. ¹H NMR (400 MHz, DMSO-d₆) δ 8.15 (s, 1H), 7.16 (d, J=8.8 Hz, 1H), 6.72 (d, J=12.8 Hz, 1H), 4.36-4.26 (m, 1H), 4.09-4.00 (m, 2H), 3.80 (d, J=6.4 Hz, 2H), 3.58-3.50 (m, 1H), 3.44-3.36 (m, 4H), 2.88-2.78 (m, 2H), 2.65 (s, 3H), 2.09-1.96 (m, 2H), 1.84-1.75 (m, 2H), 1.48-1.39 (m, 2H), 1.01 (d, J=6.8 Hz, 6H), 0.90-0.82 (m, 2H), 0.60-0.53 (m, 2H). LCMS (ESI) m/z: 484.2 [M+H]⁺.

### Example 238: 5-cyclopropyl-2-fluoro-N-((4-((1-methylazetidin-3-yl)oxy)piperidin-1-yl)sulfonyl)-4-(2,2,2-trifluoroethoxy)benzamide

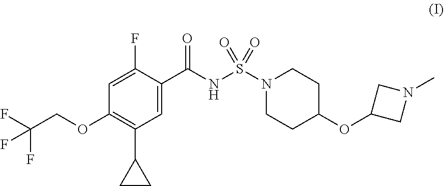 [SMILES: CN1CC(OC2CCN(S(=O)(=O)NC(=O)c3cc(C4CC4)c(OCC(F)(F)F)cc3F)CC2)C1]

Following the procedure described in Example 237 and making non-critical variations as required to replace 1-iodo-2-methylpropane with 1,1,1-trifluoro-2-iodoethane, the title compound was obtained as a white solid. ¹H NMR (400 MHz, DMSO-d₆) δ 7.20 (d, J=8.4 Hz, 1H), 6.91 (d, J=12.0 Hz, 1H), 4.83 (q, J=8.8 Hz, 2H), 4.42-4.30 (m, 1H), 4.18-4.10 (m, 2H), 3.72-3.57 (m, 5H), 2.83-2.76 (m, 2H), 2.70 (s, 3H), 2.03-1.93 (m, 1H), 1.86-1.76 (m, 2H), 1.51-1.36 (m, 2H), 0.97-0.83 (m, 2H), 0.65-0.55 (m, 2H). LCMS (ESI) m/z: 510.1 [M+H]⁺.

### Example 239: 5-chloro-4-(4-chloro-3-(trifluoromethyl)phenoxy)-2-fluoro-N-((4-((1-methylazetidin-3-yl)oxy)piperidin-1-yl)sulfonyl)benzamide

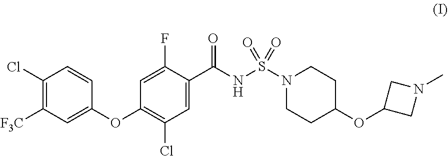 [SMILES: CN1CC(OC2CCN(S(=O)(=O)NC(=O)c3cc(Cl)c(Oc4ccc(Cl)c(C(F)(F)F)c4)cc3F)CC2)C1]

Following the procedure described in Example 237 and making non-critical variations as required to replace tert-butyl 5-cyclopropyl-2-fluoro-4-isobutoxybenzoate with tert-butyl 5-chloro-4-(4-chloro-3-(trifluoromethyl)phenoxy)-2-fluorobenzoate, the title compound was obtained as a white solid. ¹H NMR (400 MHz, DMSO-d₆) δ 7.84 (d, J=7.2 Hz, 1H), 7.73 (d, J=8.8 Hz, 1H), 7.52 (d, J=3.2 Hz, 1H), 7.34-7.27 (m, 1H), 7.13 (d, J=10.4 Hz, 1H), 4.45-4.37 (m, 1H), 4.28-4.21 (m, 2H), 3.89-3.79 (m, 2H), 3.45-3.40 (m, 2H), 2.79 (s, 3H), 2.78-2.65 (m, 3H), 1.85-1.78 (m, 2H), 1.46-1.37 (m, 2H). LCMS (ESI) m/z: 600.1 [M+H]⁺.

### Example 241: 5-chloro-4-(cyclopentylmethoxy)-2-fluoro-N-((2-fluorophenyl)sulfonyl)benzamide

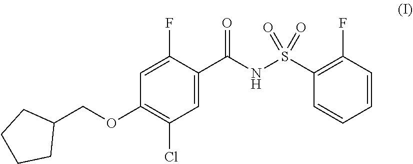 [SMILES: O=C(NS(=O)(=O)c1ccccc1F)c1cc(Cl)c(OCC2CCCC2)cc1F]

Following the procedure described in Example 204 and making non-critical variations as required to replace 4-(3-(dimethylamino)piperidin-1-yl)-2-fluorobenzenesulfonamide with 2-fluorobenzenesulfonamide, the title compound was obtained as a white solid. ¹H NMR (400 MHz, DMSO-d₆) δ 8.02-7.94 (m, 1H), 7.84-7.74 (m, 1H), 7.69 (d, J=7.2 Hz, 1H), 7.51-7.40 (m, 2H), 7.20 (d, J=12.4 Hz, 1H), 4.01 (d, J=7.2 Hz, 2H), 2.38-2.25 (m, 1H), 1.78-1.70 (m, 2H), 1.62-1.45 (m, 4H), 1.38-1.30 (m, 2H). LCMS (ESI) m/z: 430.1 [M+H]⁺.

### Example 242: 5-chloro-4-(cyclopentylmethoxy)-2-fluoro-N-(o-tolylsulfonyl)benzamide

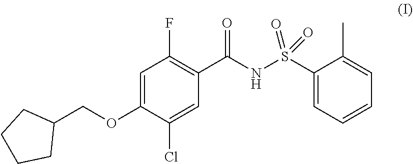 [SMILES: Cc1ccccc1S(=O)(=O)NC(=O)c1cc(Cl)c(OCC2CCCC2)cc1F]

Following the procedure described in Example 204 and making non-critical variations as required to replace 4-(3-(dimethylamino)piperidin-1-yl)-2-fluorobenzenesulfonamide with 1 2-fluorobenzenesulfonamide, the title compound was obtained as a white solid. ¹H NMR (400 MHz, DMSO-d₆) δ 12.64 (s, 1H), 8.02 (d, J=7.6 Hz, 1H), 7.68 (d, J=7.2 Hz, 1H), 7.63-7.56 (m, 1H), 7.49-7.41 (m, 2H), 7.22 (d, J=12.4 Hz, 1H), 4.01 (d, J=6.8 Hz, 2H), 2.61 (s, 3H), 2.38-2.25 (m, 1H), 1.82-1.70 (m, 2H), 1.67-1.47 (m, 4H), 1.40-1.29 (m, 2H). LCMS (ESI) m/z: 426.0 [M+H]⁺.

### Example 243: 5-chloro-4-(cyclopentylmethoxy)-N-((4-ethylphenyl)sulfonyl)-2-fluorobenzamide

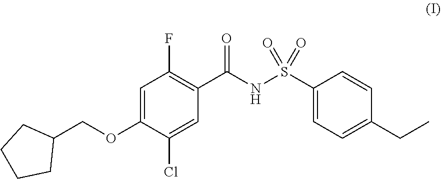 [SMILES: CCc1ccc(S(=O)(=O)NC(=O)c2cc(Cl)c(OCC3CCCC3)cc2F)cc1]

Following the procedure described in Example 204 and making non-critical variations as required to replace 4-(3-(dimethylamino)piperidin-1-yl)-2-fluorobenzenesulfonamide with 4-ethylbenzenesulfonamide, the title compound was obtained as a white solid. ¹H NMR (400 MHz, DMSO-d₆) δ 12.41 (s, 1H), 7.89 (d, J=8.4 Hz, 2H), 7.68 (d, J=7.6 Hz, 1H), 7.49 (d, J=8.4 Hz, 2H), 7.21 (d, J=12.4 Hz, 1H), 4.01 (d, J=7.2 Hz, 2H), 2.75-2.65 (m, 2H), 2.40-2.25 (m, 1H), 1.82-1.69 (m, 2H), 1.66-1.47 (m, 4H), 1.41-1.26 (m, 2H), 1.21 (t, J=7.6 Hz, 3H). LCMS (ESI) m/z: 440.2 [M+H]⁺.

### Example 244: 5-chloro-4-(cyclopentylmethoxy)-N-((3-ethylphenyl)sulfonyl)-2-fluorobenzamide

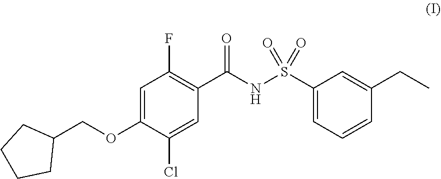 [SMILES: CCc1cccc(S(=O)(=O)NC(=O)c2cc(Cl)c(OCC3CCCC3)cc2F)c1]

Following the procedure described in Example 204 and making non-critical variations as required to replace 4-(3-(dimethylamino)piperidin-1-yl)-2-fluorobenzenesulfonamide with 3-ethylbenzenesulfonamide, the title compound was obtained as a white solid. ¹H NMR (400 MHz, DMSO-d₆) δ 12.45 (s, 1H), 7.83-7.77 (m, 2H), 7.68 (d, J=7.6 Hz, 1H), 7.62-7.52 (m, 2H), 7.22 (d, J=12.4 Hz, 1H), 4.01 (d, J=6.8 Hz, 2H), 2.72 (q, J=7.2 Hz, 2H), 2.40-2.28 (m, 1H), 1.82-1.69 (m, 2H), 1.66-1.47 (m, 4H), 1.39-1.23 (m, 2H), 1.21 (t, J=7.2 Hz, 3H). LCMS (ESI) m/z: 440.0 [M+H]⁺.

### Example 245: 5-chloro-4-(cyclopentylmethoxy)-2-fluoro-N-((3-methoxyphenyl)sulfonyl)benzamide

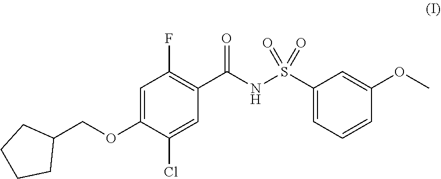 [SMILES: COc1cccc(S(=O)(=O)NC(=O)c2cc(Cl)c(OCC3CCCC3)cc2F)c1]

Following the procedure described in Example 204 and making non-critical variations as required to replace 4-(3-(dimethylamino)piperidin-1-yl)-2-fluorobenzenesulfonamide with 3-methoxybenzenesulfonamide, the title compound was obtained as a white solid. ¹H NMR (400 MHz, DMSO-d₆) δ 12.45 (s, 1H), 7.69 (d, J=7.62 Hz, 1H), 7.65-7.53 (m, 2H), 7.45 (s, 1H), 7.30-7.23 (m, 1H), 7.22 (d, J=12.8 Hz, 1H), 4.02 (d, J=6.8 Hz, 2H), 3.84 (s, 3H), 2.35-2.27 (m, 1H), 1.81-1.70 (m, 2H), 1.68-1.46 (m, 4H), 1.39-1.28 (m, 2H). LCMS (ESI) m/z: 442.1 [M+H]⁺.

### Example 246: 5-chloro-4-(cyclopentylmethoxy)-2-fluoro-N-((2-methoxyphenyl)sulfonyl)benzamide

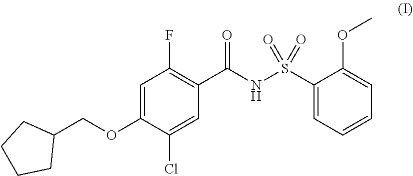 [SMILES: COc1ccccc1S(=O)(=O)NC(=O)c1cc(Cl)c(OCC2CCCC2)cc1F]

Following the procedure described in Example 204 and making non-critical variations as required to replace 4-(3-(dimethylamino)piperidin-1-yl)-2-fluorobenzenesulfonamide with 2-methoxybenzenesulfonamide, the title compound was obtained as a white solid. ¹H NMR (400 MHz, DMSO-d₆) δ 12.46 (s, 1H), 7.90 (d, J=8.0 Hz, 1H), 7.72-7.61 (m, 2H), 7.26 (d, J=8.4 Hz, 1H), 7.22-7.08 (m, 2H), 4.01 (d, J=6.8 Hz, 2H), 3.90 (s, 3H), 2.40-2.28 (m, 1H), 1.82-1.70 (m, 2H), 1.67-1.46 (m, 4H), 1.40-1.27 (m, 2H). LCMS (ESI) m/z: 442.1 [M+H]⁺.

### Example 247: 5-chloro-4-(cyclopentylmethoxy)-2-fluoro-N-(isopropylsulfonyl)benzamide

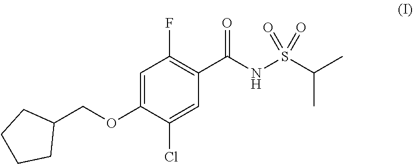 [SMILES: CC(C)S(=O)(=O)NC(=O)c1cc(Cl)c(OCC2CCCC2)cc1F]

Following the procedure described in Example 204 and making non-critical variations as required to replace 4-(3-(dimethylamino)piperidin-1-yl)-2-fluorobenzenesulfonamide with propane-2-sulfonamide, the title compound was obtained as a white solid. ¹H NMR (400 MHz, DMSO-d₆) δ 11.95 (s, 1H), 7.74 (d, J=7.6 Hz, 1H), 7.25 (d, J=12.4 Hz, 1H), 4.03 (d, J=6.8 Hz, 2H), 3.85-3.75 (m, 1H), 2.38-2.27 (m, 1H), 1.82-1.71 (m, 2H), 1.67-1.48 (m, 4H), 1.41-1.33 (m, 2H), 1.31 (d, J=6.8 Hz, 6H). LCMS (ESI) m/z: 378.1 [M+H]⁺.

### Example 248 and Example 249: (S)-5-chloro-4-(cyclopentylmethoxy)-2-fluoro-N-((1-methoxypropan-2-yl)sulfonyl)benzamide, and (R)-5-chloro-4-(cyclopentylmethoxy)-2-fluoro-N-((1-methoxypropan-2-yl)sulfonyl)-benzamide

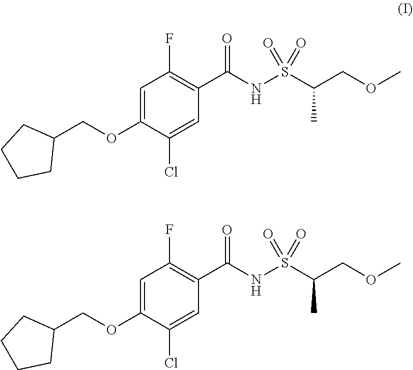 [SMILES: COC[C@H](C)S(=O)(=O)NC(=O)c1cc(Cl)c(OCC2CCCC2)cc1F ; COC[C@@H](C)S(=O)(=O)NC(=O)c1cc(Cl)c(OCC2CCCC2)cc1F]

Following the procedure described in Example 204 and making non-critical variations as required to replace 4-(3-(dimethylamino)piperidin-1-yl)-2-fluorobenzenesulfonamide with 1-methoxypropane-2-sulfonamide, 5-chloro-4-(cyclopentylmethoxy)-2-fluoro-N-((1-methoxypropan-2-yl)sulfonyl)benzamide was obtained as a yellow solid. 5-chloro-4-(cyclopentylmethoxy)-2-fluoro-N-((1-methoxypropan-2-yl)sulfonyl)benzamide (150 mg, 0.37 mmol) was separated by using chiral SFC (DAICEL CHIRALPAK AY-H (250 mm*30 mm, 5 um), Supercritical CO₂/EtOH+0.1% NH₃·H₂O=40/60; 60 mL/min) to afford (S)-5-chloro-4-(cyclopentylmethoxy)-2-fluoro-N-((1-methoxypropan-2-yl)sulfonyl)benzamide (19 mg, second peak) as a yellow solid and (R)-5-chloro-4-(cyclopentylmethoxy)-2-fluoro-N-((1-methoxypropan-2-yl)sulfonyl)benzamide (30 mg, first peak) as a yellow solid. Absolute configuration was arbitrarily assigned to each enantiomer. Example 248:: ¹H NMR (400 MHz, DMSO-d₆) δ 12.03 (s, 1H), 7.72 (d, J=7.6 Hz, 1H), 7.21 (d, J=12.4 Hz, 1H), 4.02 (d, J=6.8 Hz, 2H), 3.92-3.80 (m, 1H), 3.75-3.65 (m, 1H), 3.58-3.47 (m, 1H), 3.23 (s, 3H), 2.40-2.27 (m, 1H), 1.83-1.70 (m, 2H), 1.66-1.48 (m, 4H), 1.41-1.33 (m, 2H), 1.31 (d, J=6.8 Hz, 3H). LCMS (ESI) m/z: 408.2 [M+H]⁺. Example 249:: ¹H NMR (400 MHz, DMSO-d₆) δ 12.04 (s, 1H), 7.72 (d, J=7.6 Hz, 1H), 7.21 (d, J=12.4 Hz, 1H), 4.02 (d, J=6.8 Hz, 2H), 3.92-3.80 (m, 1H), 3.75-3.65 (m, 1H), 3.58-3.47 (m, 1H), 3.23 (s, 3H), 2.40-2.27 (m, 1H), 1.83-1.70 (m, 2H), 1.66-1.48 (m, 4H), 1.41-1.33 (m, 2H), 1.31 (d, J=6.8 Hz, 3H). LCMS (ESI) m/z: 408.1[M+H]⁺.

### Example 250 and Example 251: 5-chloro-4-(cyclopentylmethoxy)-2-fluoro-N-(((1S,2S)-2-methylcyclopropyl)sulfonyl)benzamide, and 5-chloro-4-(cyclopentylmethoxy)-2-fluoro-N-(((1R,2R)-2-methylcyclopropyl)sulfonyl)-benzamide

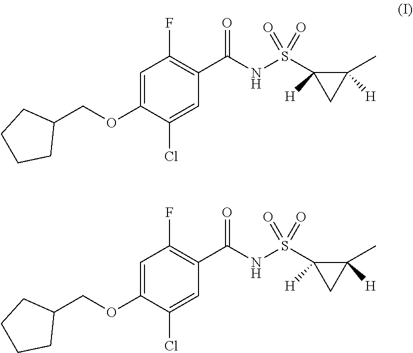 [SMILES: C[C@H]1C[C@@H]1S(=O)(=O)NC(=O)c1cc(Cl)c(OCC2CCCC2)cc1F ; C[C@@H]1C[C@H]1S(=O)(=O)NC(=O)c1cc(Cl)c(OCC2CCCC2)cc1F]

Following the procedure described in Example 204 and making non-critical variations as required to replace 4-(3-(dimethylamino)piperidin-1-yl)-2-fluorobenzenesulfonamide with (1S,2S)-2-methylcyclopropane-1-sulfonamide, 5-chloro-4-(cyclopentylmethoxy)-2-fluoro-N-((trans-2-methylcyclopropyl)sulfonyl)benzamide was obtained as a white solid. 5-chloro-4-(cyclopentylmethoxy)-2-fluoro-N-((trans-2-methylcyclopropyl)sulfonyl)benzamide (240 mg, 0.62 mmol) was separated by using chiral SFC (DAICEL CHIRALPAK IC (250 mm*30 mm, 10 um), Supercritical CO₂/EtOH+0.1% NH₃·H₂O=70/30; 70 mL/min) to afford 5-chloro-4-(cyclopentylmethoxy)-2-fluoro-N-(((1S,2S)-2-methylcyclopropyl)sulfonyl)benzamide (65 mg, first peak) as a white solid and 5-chloro-4-(cyclopentylmethoxy)-2-fluoro-N-(((1R,2R)-2-methylcyclopropyl)sulfonyl)benzamide (72 mg, second peak) as a white solid. Absolute configuration was arbitrarily assigned to each enantiomer. Example 250: ¹H NMR (400 MHz, DMSO-d₆) δ 12.00 (s, 1H), 7.74 (d, J=7.6 Hz, 1H), 7.24 (d, J=12.8 Hz, 1H), 4.04 (d, J=6.8 Hz, 2H), 2.85-2.75 (m, 1H), 2.40-2.28 (m, 1H), 1.83-1.69 (m, 2H), 1.67-1.48 (m, 5H), 1.41-1.23 (m, 3H), 1.10 (d, J=6.0 Hz, 3H), 1.03-0.91 (m, 1H). LCMS (ESI) m/z: 390.0 [M+H]⁺. Example 251: ¹H NMR (400 MHz, DMSO-d₆) δ 12.00 (s, 1H), 7.74 (d, J=7.6 Hz, 1H), 7.23 (d, J=12.8 Hz, 1H), 4.03 (d, J=6.8 Hz, 2H), 2.85-2.75 (m, 1H), 2.40-2.28 (m, 1H), 1.85-1.68 (m, 2H), 1.68-1.45 (m, 5H), 1.42-1.22 (m, 3H), 1.10 (d, J=6.0 Hz, 3H), 1.03-0.91 (m, 1H). LCMS (ESI) m/z: 390.0 [M+H]⁺.

### Example 252: 5-chloro-4-(cyclopentylmethoxy)-2-fluoro-N-((4-methylthiophen-2-yl)sulfonyl)benzamide

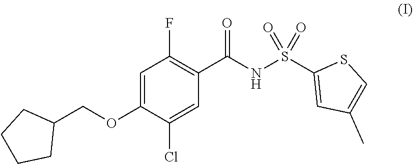 [SMILES: Cc1csc(S(=O)(=O)NC(=O)c2cc(Cl)c(OCC3CCCC3)cc2F)c1]

To a solution of NaH (41 mg, 1.02 mmol, 60% in mineral oil) in DMF (2 mL) was added 5-chloro-4-(cyclopentylmethoxy)-2-fluorobenzamide (332 mg, 1.22 mmol) at 0° C. under nitrogen atmosphere. After stirring at 0° C. for 10 min, 4-methylthiophene-2-sulfonyl chloride (200 mg, 1.02 mmol) in DMF (2 mL) was added at 0° C. After stirring at room temperature for 16 h, the reaction was quenched with water (30 mL) and extracted with EtOAc (60 mL×3). The combined organic lawyers were washed with brine (30 mL×5), dried over anhydrous Na₂SO₄, filtered and concentrated in vacuo. The crude residue was purified by reverse phase chromatography (acetonitrile 65-95%/0.2% formic acid in water) to afford the title compound (164 mg, 37%) as a white solid. ¹H NMR (400 MHz, DMSO-d₆) δ 12.61 (s, 1H), 7.76-7.62 (m, 3H), 7.23 (d, J=12.4 Hz, 1H), 4.02 (d, J=6.8 Hz, 2H), 2.37-2.28 (m, 1H), 2.25 (s, 3H), 1.82-1.72 (m, 2H), 1.67-1.48 (m, 4H), 1.38-1.29 (m, 2H). LCMS (ESI) m/z: 431.9 [M+H]⁺.

### Example 253: 5-chloro-4-(cyclopentylmethoxy)-2-fluoro-N-((3-(3-(trifluoromethyl)phenoxy)propyl)sulfonyl)benzamide

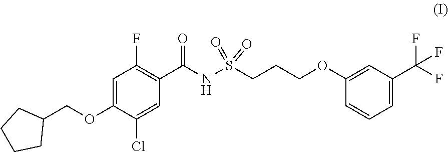 [SMILES: O=C(NS(=O)(=O)CCCOc1cccc(C(F)(F)F)c1)c1cc(Cl)c(OCC2CCCC2)cc1F]

Following the procedure described in Example 252 and making non-critical variations as required to replace 4-methylthiophene-2-sulfonyl chloride with 3-(3-(trifluoromethyl)-phenoxy)propane-1-sulfonyl chloride, the title compound was obtained as a white solid. ¹H NMR (400 MHz, DMSO-d₆) δ 12.08 (s, 1H), 7.68 (d, J=7.2 Hz, 1H), 7.52-7.46 (m, 1H), 7.31-7.16 (m, 4H), 4.19 (t, J=6.0 Hz, 2H), 4.02 (d, J=6.8 Hz, 2H), 3.75-3.65 (m, 2H), 2.45-2.35 (m, 1H), 2.25-2.13 (m, 2H), 1.85-1.75 (m, 2H), 1.68-1.48 (m, 4H), 1.42-1.29 (m, 2H). LCMS (ESI) m/z: 538.1 [M+H]⁺.

### Example 254 and Example 255: (S)—N-(sec-butylsulfonyl)-5-chloro-4-(cyclopentylmethoxy)-2-fluorobenzamide, and (R)—N-(sec-butylsulfonyl)-5-chloro-4-(cyclopentylmethoxy)-2-fluorobenzamide

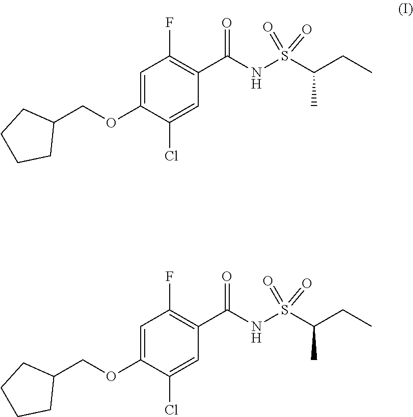 [SMILES: CC[C@H](C)S(=O)(=O)NC(=O)c1cc(Cl)c(OCC2CCCC2)cc1F ; CC[C@@H](C)S(=O)(=O)NC(=O)c1cc(Cl)c(OCC2CCCC2)cc1F]

Following the procedure described in Example 252 and making non-critical variations as required to replace 4-methylthiophene-2-sulfonyl chloride with (1S,2S)-2-methylcyclopropane-1-sulfonamide, N-(sec-butylsulfonyl)-5-chloro-4-(cyclopentylmethoxy)-2-fluorobenzamide was obtained as a white solid. N-(sec-butylsulfonyl)-5-chloro-4-(cyclopentylmethoxy)-2-fluorobenzamide (120 mg, 0.31 mmol) was separated by using chiral SFC (DAICEL CHIRALCEL OD-H (250 mm*30 mm, 5 um), Supercritical CO₂/MeOH+0.1% NH₃·H₂O=20/80; 60 mL/min) to afford (S)—N-(sec-butylsulfonyl)-5-chloro-4-(cyclopentylmethoxy)-2-fluorobenzamide (50 mg, second peak) as a white solid and (R)—N-(sec-butylsulfonyl)-5-chloro-4-(cyclopentylmethoxy)-2-fluorobenzamide (50 mg, first peak) as a white solid. Absolute configuration was arbitrarily assigned to each enantiomer. Example 254: ¹H NMR (400 MHz, DMSO-d₆) δ 11.94 (s, 1H), 7.73 (d, J=7.2 Hz, 1H), 7.23 (d, J=12.0 Hz, 1H), 4.03 (d, J=6.8 Hz, 2H), 3.58-3.46 (m, 1H), 2.38-2.27 (m, 1H), 2.02-1.89 (m, 1H), 1.83-1.72 (m, 2H), 1.64-1.48 (m, 5H), 1.39-1.30 (m, 2H), 1.30 (d, J=7.2 Hz, 3H), 0.99 (t, J=7.2 Hz, 3H). LCMS (ESI) m/z: 392.1 [M+H]⁺. Example 255: ¹H NMR (400 MHz, DMSO-d₆) δ 11.95 (s, 1H), 7.73 (d, J=7.2 Hz, 1H), 7.21 (d, J=12.0 Hz, 1H), 4.03 (d, J=6.8 Hz, 2H), 3.59-3.47 (m, 1H), 2.40-2.29 (m, 1H), 1.99-1.89 (m, 1H), 1.83-1.72 (m, 2H), 1.67-1.47 (m, 5H), 1.41-1.32 (m, 2H), 1.29 (d, J=6.8 Hz, 3H), 0.99 (t, J=7.2 Hz, 3H). LCMS (ESI) m/z: 392.2 [M+H]⁺.

### Example 256: 5-chloro-4-(cyclopentylmethoxy)-2-fluoro-N-(spiro[2.3]hexan-1-ylsulfonyl)benzamide

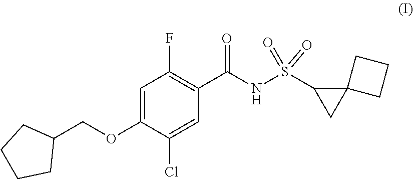 [SMILES: O=C(NS(=O)(=O)C1CC12CCC2)c1cc(Cl)c(OCC2CCCC2)cc1F]

Following the procedure described in Example 252 and making non-critical variations as required to replace 4-methylthiophene-2-sulfonyl chloride with spiro[2.3]hexane-1-sulfonyl chloride, the title compound was obtained as a white solid. ¹H NMR (400 MHz, DMSO-d₆) δ 12.10 (s, 1H), 7.74 (d, J=7.6 Hz, 1H), 7.25 (d, J=12.0 Hz, 1H), 4.04 (d, J=6.8 Hz, 2H), 3.02-2.93 (m, 1H), 2.64-2.55 (m, 1H), 2.42-2.26 (m, 1H), 2.26-2.20 (m, 1H), 2.19-2.10 (m, 1H), 2.08-2.00 (m, 2H), 1.98-1.89 (m, 1H), 1.82-1.72 (m, 2H), 1.66-1.48 (m, 4H), 1.39-1.28 (m, 4H). LCMS (ESI) m/z: 416.2 [M+H]⁺.

### Example 257: N-(5-((2-chlorobenzyl)thio)pyridin-2-yl)-4-(((1S,2S)-2-(dimethylamino)cyclohexyl)oxy)-2-fluorobenzenesulfonamide

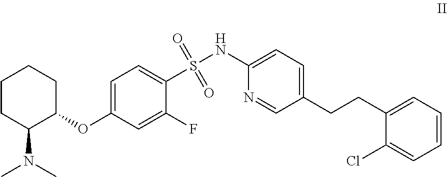 [SMILES: CN(C)[C@H]1CCCC[C@@H]1Oc1ccc(S(=O)(=O)Nc2ccc(CCc3ccccc3Cl)cn2)c(F)c1]

### Step 1: (1S,2S)-2-(dimethylamino)cyclohexanol

To a solution of (1S,2S)-2-aminocyclohexanol (5.0 g, 43.41 mmol) in formic acid (16 mL, 433 mmol) was added formaldehyde (32 mL, 433 mmol, 37% in water). The reaction mixture was stirred at 110° C. for 3 h. After cooling to room temperature, the mixture was concentrated in vacuo. The residue was dissolved in EtOAc (50 mL) and basified to pH=13 by anhydrous NaOH (5 M). The aqueous phase was extracted with EtOAc (75 mL×2). The combined organic layers were dried over anhydrous Na₂SO₄, filtered and concentrated in vacuo to give the title compound (7.8 g, crude) as yellow oil that required no further purification. ¹H NMR (400 MHz, CDCl₃) δ 3.89 (s, 1H), 3.37-3.24 (m, 1H), 2.23 (s, 6H), 2.18-2.09 (m, 1H), 2.08-2.03 (m, 1H), 1.81-1.61 (m, 3H), 1.24-1.11 (m, 4H).

### Step 2: (1S,2S)-2-(3-fluorophenoxy)-N,N-dimethylcyclohexanamine

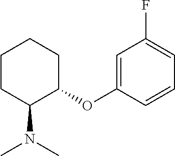 [SMILES: CN(C)[C@H]1CCCC[C@@H]1Oc1cccc(F)c1]

To a stirred solution of NaH (600.0 mg, 15.00 mmol, 60% in mineral oil) in 1-methyl-2-pyrrolidinone (20 mL) was added (1S,2S)-2-(dimethylamino)cyclohexanol (2.0 g, 13.96 mmol) slowly at 0° C. The mixture was stirred at room temperature for 20 min and then 1,3-difluorobenzene (1.91 g, 16.76 mmol) was added. The reaction was stirred at 100° C. for an additional 2 h. After cooling to room temperature, the mixture was quenched with brine (50 mL) and extracted by EtOAc (100 mL). The organic layer was washed with brine (100 mL×5), dried over anhydrous Na₂SO₄, filtered and concentrated in vacuo to give the title compound (1.9 g, crude) as yellow oil that required no further purification. ¹H NMR (400 MHz, CDCl₃) δ 7.26-7.16 (m, 1H), 6.78-6.68 (m, 1H), 6.67-6.57 (m, 2H), 4.31-4.18 (m, 1H), 2.72-2.68 (m, 1H), 2.45 (s, 6H), 2.21-2.18 (m, 1H), 1.96-1.85 (m, 1H), 1.83-1.70 (m, 2H), 1.36-1.25 (m, 4H). LCMS M/Z (M+H) 238.2.

### Step 3: 4-(((1S,2S)-2-(dimethylamino)cyclohexyl)oxy)-2-fluorobenzenesulfonic acid

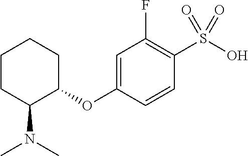 [SMILES: CN(C)[C@H]1CCCC[C@@H]1Oc1ccc(S(=O)(=O)O)c(F)c1]

To a solution of (1S,2S)-2-(3-fluorophenoxy)-N,N-dimethyl-cyclohexanamine (3.0 g, 12.64 mmol) in anhydrous DCM (60 mL) was added chlorosulfonic acid (2.18 mL, 31.60 mmol) at 0° C. slowly. The mixture was stirred at 0° C. for 3 h under a nitrogen atmosphere. The reaction was quenched by water (30 mL). The aqueous phase was neutralized by 28% ammonium hydroxide to pH=9 and then the mixture was concentrated in vacuo to remove most organic solvent. The residue was purified by reverse phase chromatography (acetonitrile 0-30%/0.05% NH₄OH in water) to give the title compound (803 mg, 20%) as a white solid. ¹H NMR (400 MHz, DMSO-d₆) δ 7.58 (dd, J=8.8, 8.8 Hz, 1H), 6.88 (dd, J=11.6, 2.4 Hz, 1H), 6.80 (dd, J=8.8, 2.4 Hz, 1H), 4.70-4.59 (m, 1H), 3.49-3.47 (m, 1H), 2.71 (s, 6H), 2.20-2.12 (m, 1H), 2.07-2.00 (m, 1H), 1.82-1.75 (m, 1H), 1.70-1.60 (m, 1H), 1.54-1.36 (m, 2H), 1.35-1.25 (m, 2H). LCMS M/Z (M+H) 318.2.

### Step 4: 4-(((1S,2S)-2-(dimethylamino)cyclohexyl)oxy)-2-fluorobenzene-1-sulfonyl chloride

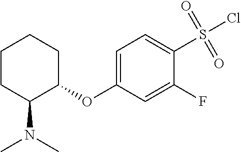 [SMILES: CN(C)[C@H]1CCCC[C@@H]1Oc1ccc(S(=O)(=O)Cl)c(F)c1]

To a solution of 4-(((1S,2S)-2-(dimethylamino)cyclohexyl)oxy)-2-fluorobenzenesulfonic acid (48 mg, 0.15 mmol) in DCM (2.5 mL) was added SOCl₂ (179 mg, 1.5 mmol) and two drops of DMF at 0° C. The mixture was stirred at room temperature for 2 h. The reaction was concentrated in vacuo to afford the title compound (40 mg, crude) as colorless oil that required no further purification. LCMS (ESI) m/z: 336.0 [M+H]⁺.

### Step 5: N-(5-((2-chlorobenzyl)thio)pyridin-2-yl)-4-(((1S,2S)-2-(dimethylamino)cyclohexyl)oxy)-2-fluorobenzenesulfonamide

To a solution of 4-(((1S,2S)-2-(dimethylamino)cyclohexyl)oxy)-2-fluorobenzene-1-sulfonyl chloride (40 mg, 0.13) and 5-((2-chlorobenzyl)thio)pyridin-2-amine (25 mg, 0.10 mmol) in pyridine (1 mL) was added 4A MS under a nitrogen atmosphere. The mixture was stirred at 80° C. for 5 h. The mixture was concentrated in vacuo and the crude residue was purified by reverse phase chromatography (acetonitrile 30-70%/0.2% HCOOH in water) to afford the title compound (14 mg, 44%). LCMS (ESI) m/z: 550.1 [M+H]⁺.

### Example 258: N-(5-(4-chlorophenoxy)pyridin-2-yl)-4-(((1S,2S)-2-(dimethylamino)cyclohexyl)oxy)-2-fluorobenzenesulfonamide

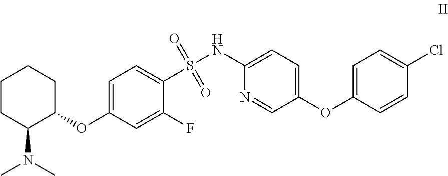 [SMILES: CN(C)[C@H]1CCCC[C@@H]1Oc1ccc(S(=O)(=O)Nc2ccc(Oc3ccc(Cl)cc3)cn2)c(F)c1]

Following the procedure described in Example 257 and making non-critical variations as required to replace 5-((2-chlorobenzyl)thio)pyridin-2-amine with 5-(4-chlorophenoxy)pyridin-2-amine. The title compound was obtained. LCMS (ESI) m/z: 520.1 [M+H]⁺.

### Example 259: 5-chloro-4-(cyclopentylmethoxy)-2-fluoro-N-((1-(3-(trifluoromethyl)phenyl)-1H-pyrazol-4-yl)sulfonyl)benzamide

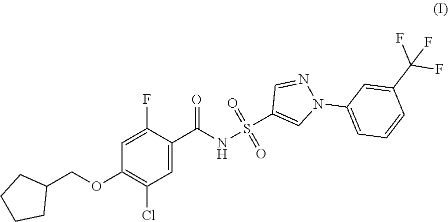 [SMILES: O=C(NS(=O)(=O)c1cnn(-c2cccc(C(F)(F)F)c2)c1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 1-(3-(trifluoromethyl)phenyl)-1H-pyrazole-4-sulfonamide. The title compound was obtained. LCMS (ESI) m/z: 546.1 [M+H]⁺.

### Example 260: 5-chloro-4-(cyclopentylmethoxy)-2-fluoro-N-((4-((3-methylbenzyl)oxy)phenyl)sulfonyl)benzamide

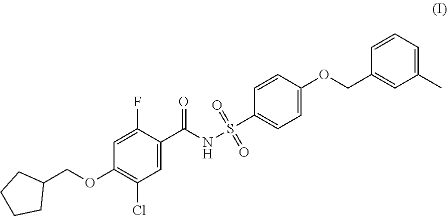 [SMILES: Cc1cccc(COc2ccc(S(=O)(=O)NC(=O)c3cc(Cl)c(OCC4CCCC4)cc3F)cc2)c1]

Following the procedure described in Example 204 and making non-critical variations as required to replace 4-(((1S,2S)-2-(dimethylamino)cyclohexyl)amino)-2,6-difluorobenzenesulfonamide with 4-((3-methylbenzyl)oxy)benzenesulfonamide. The title compound was obtained. LCMS (ESI) m/z: 532.1 [M+H]⁺.

### Example 261: 5-chloro-4-(cyclopentylmethoxy)-2-fluoro-N-((5,6,7,8-tetrahydronaphthalen-2-yl)sulfonyl)benzamide

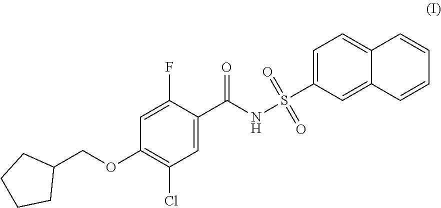 [SMILES: O=C(NS(=O)(=O)c1ccc2ccccc2c1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 5,6,7,8-tetrahydronaphthalene-2-sulfonamide. The title compound was obtained. LCMS (ESI) m/z: 466.1 [M+H]⁺.

### Example 262: 5-chloro-4-(cyclopentylmethoxy)-N-((2,3-dihydro-1H-inden-5-yl)sulfonyl)-2-fluorobenzamide

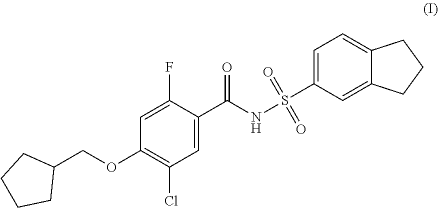 [SMILES: O=C(NS(=O)(=O)c1ccc2c(c1)CCC2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 2,3-dihydro-1H-indene-5-sulfonamide. The title compound was obtained. LCMS (ESI) m/z: 452.1 [M+H]⁺.

### Example 263: 5-chloro-4-(cyclopentylmethoxy)-2-fluoro-N-((4-phenoxyphenyl)sulfonyl)benzamide

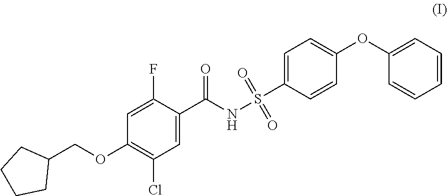 [SMILES: O=C(NS(=O)(=O)c1ccc(Oc2ccccc2)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 4-phenoxybenzenesulfonamide. The title compound was obtained. LCMS (ESI) m/z: 504.1 [M+H]⁺.

### Example 264: 5-chloro-N-((4-((2-chlorobenzyl)oxy)phenyl)sulfonyl)-4-(cyclopentylmethoxy)-2-fluorobenzamide

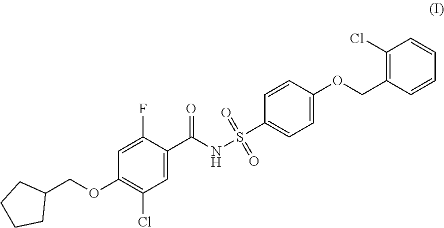 [SMILES: O=C(NS(=O)(=O)c1ccc(OCc2ccccc2Cl)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzene-sulfonamide with 4-((2-chlorobenzyl)oxy)benzenesulfonamide. The title compound was obtained. LCMS (ESI) m/z: 552.1 [M+H]⁺.

### Example 265: 5-chloro-N-(chroman-6-ylsulfonyl)-4-(cyclopentylmethoxy)-2-fluorobenzamide

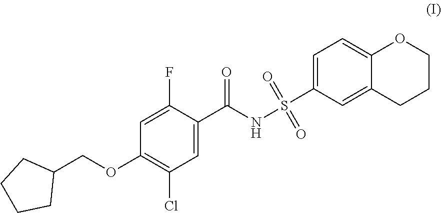 [SMILES: O=C(NS(=O)(=O)c1ccc2c(c1)CCCO2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with chroman-6-sulfonamide. The title compound was obtained. LCMS (ESI) m/z: 468.1 [M+H]⁺.

### Example 266: 5-chloro-4-(cyclopentylmethoxy)-N-(((2,3-dihydro-1H-inden-2-yl)methyl)sulfonyl)-2-fluorobenzamide

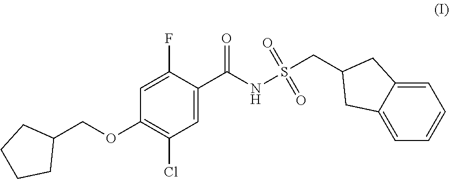 [SMILES: O=C(NS(=O)(=O)CC1Cc2ccccc2C1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with (2,3-dihydro-1H-inden-2-yl)methanesulfonamide. The title compound was obtained. LCMS (ESI) m/z: 466.1 [M+H]⁺.

### Example 267: 5-chloro-4-(cyclopentylmethoxy)-2-fluoro-N-((1,2,3,4-tetrahydroquinolin-7-yl)sulfonyl)benzamide

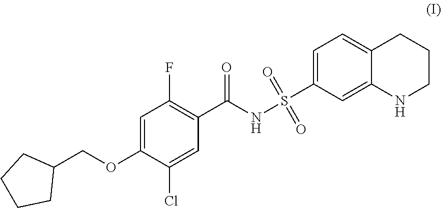 [SMILES: O=C(NS(=O)(=O)c1ccc2c(c1)NCCC2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 1,2,3,4-tetrahydroquinoline-7-sulfonamide. The title compound was obtained. LCMS (ESI) m/z: 467.1 [M+H]⁺.

### Example 268: 5-chloro-4-(cyclopentylmethoxy)-N-((2,3-difluorobenzyl)sulfonyl)-2-fluorobenzamide

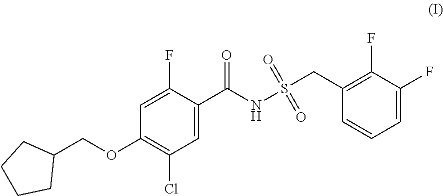 [SMILES: O=C(NS(=O)(=O)Cc1cccc(F)c1F)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with (2,3-difluorophenyl)methanesulfonamide. The title compound was obtained. LCMS (ESI) m/z: 462.0 [M+H]⁺.

### Example 269: 5-chloro-4-(cyclopentylmethoxy)-2-fluoro-N-((1-(2-fluorophenyl)-1H-pyrazol-4-yl)sulfonyl)benzamide

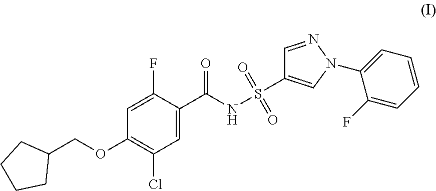 [SMILES: O=C(NS(=O)(=O)c1cnn(-c2ccccc2F)c1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 1-(2-fluorophenyl)-1H-pyrazole-4-sulfonamide. The title compound was obtained. LCMS (ESI) m/z: 496.1 [M+H]⁺.

### Example 270: (S)-5-chloro-4-(cyclopentylmethoxy)-2-fluoro-N-((2-methyl-2,3-dihydrobenzofuran-5-yl)sulfonyl)benzamide

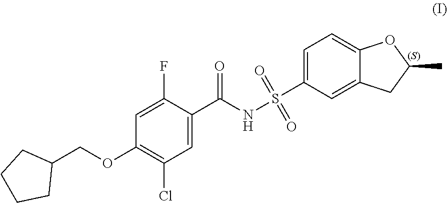

Following the procedure described in Example 204 and making non-critical variations as required to replace 4-(((1S,2S)-2-(dimethylamino)cyclohexyl)amino)-2,6-difluorobenzenesulfonamide with (S)-2-methyl-2,3-dihydrobenzofuran-5-sulfonamide. The title compound was obtained. LCMS (ESI) m/z: 468.1 [M+H]⁺.

### Example 271: 5-chloro-4-(cyclopentylmethoxy)-N-((2,3-dihydrobenzofuran-5-yl)sulfonyl)-2-fluorobenzamide

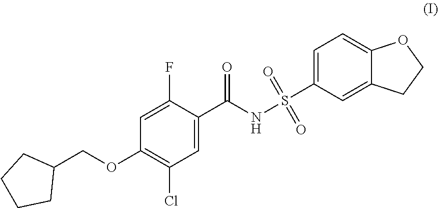 [SMILES: O=C(NS(=O)(=O)c1ccc2c(c1)CCO2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 2,3-dihydrobenzofuran-5-sulfonamide. The title compound was obtained. LCMS (ESI) m/z: 454.1 [M+H]⁺.

### Example 272: 5-chloro-N-((4-cyano-2-fluorophenyl)sulfonyl)-4-(cyclopentylmethoxy)-2-fluorobenzamide

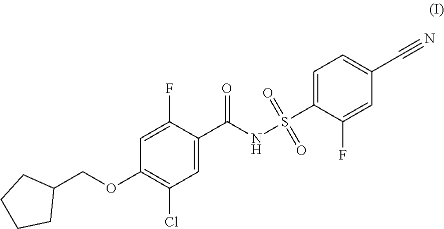 [SMILES: N#Cc1ccc(S(=O)(=O)NC(=O)c2cc(Cl)c(OCC3CCCC3)cc2F)c(F)c1]

Following the procedure described in Example 204 and making non-critical variations as required to replace 4-(((1S,2S)-2-(dimethylamino)cyclohexyl)amino)-2,6-difluorobenzenesulfonamide with 4-cyano-2-fluorobenzenesulfonamide. The title compound was obtained. LCMS (ESI) m/z: 455.0 [M+H]⁺.

### Example 273: 5-chloro-4-(cyclopentylmethoxy)-2-fluoro-N-((quinolin-6-ylmethyl)sulfonyl)benzamide

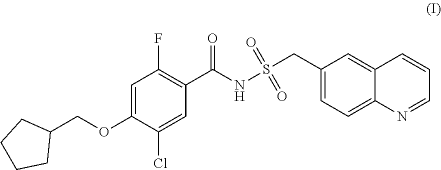 [SMILES: O=C(NS(=O)(=O)Cc1ccc2ncccc2c1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with quinolin-6-ylmethanesulfonamide. The title compound was obtained. LCMS (ESI) m/z: 477.1 [M+H]⁺.

### Example 274: N-((2-(benzyloxy)ethyl)sulfonyl)-5-chloro-4-(cyclopentylmethoxy)-2-fluorobenzamide

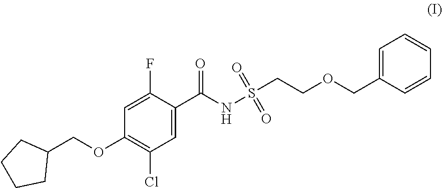 [SMILES: O=C(NS(=O)(=O)CCOCc1ccccc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 2-(benzyloxy)ethanesulfonamide. The title compound was obtained. LCMS (ESI) m/z: 470.2 [M+H]⁺.

### Example 275: 5-chloro-4-(cyclopentylmethoxy)-N-((3,4-dihydronaphthalen-2-yl)sulfonyl)-2-fluorobenzamide

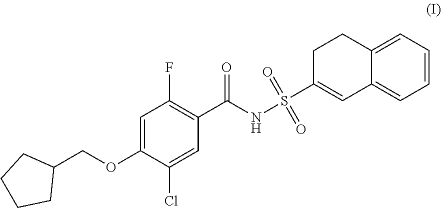 [SMILES: O=C(NS(=O)(=O)C1=Cc2ccccc2CC1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzene-sulfonamide with 3,4-dihydronaphthalene-2-sulfonamide. The title compound was obtained. LCMS (ESI) m/z: 464.2 [M+H]⁺.

### Example 276: 5-chloro-4-(cyclopentylmethoxy)-N-((4-cyclopropoxyphenyl)sulfonyl)-2-fluorobenzamide

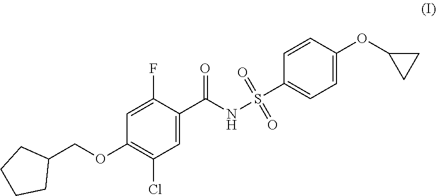 [SMILES: O=C(NS(=O)(=O)c1ccc(OC2CC2)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzene-sulfonamide with 4-cyclopropoxybenzenesulfonamide. The title compound was obtained. LCMS (ESI) m/z: 468.1 [M+H]⁺.

### Example 277: (S)-5-(N-(5-chloro-4-(cyclopentylmethoxy)-2-fluorobenzoyl)sulfamoyl)-2,3-dihydrobenzofuran-2-carboxamide

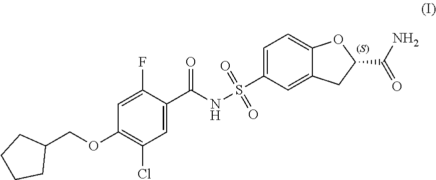

Following the procedure described in Example 204 and making non-critical variations as required to replace 4-(((1S,2S)-2-(dimethylamino)cyclohexyl)amino)-2,6-difluorobenzenesulfonamide with (S)-5-sulfamoyl-2,3-dihydrobenzofuran-2-carboxamide. The title compound was obtained. LCMS (ESI) m/z: 497.2 [M+H]⁺.

### Example 278: 5-chloro-4-(cyclopentylmethoxy)-2-fluoro-N-((1-fluoronaphthalen-2-yl)sulfonyl)benzamide

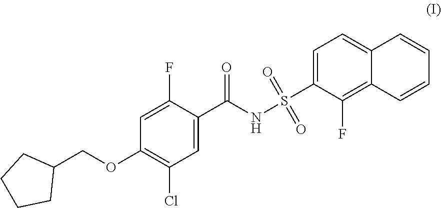 [SMILES: O=C(NS(=O)(=O)c1ccc2ccccc2c1F)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 1-fluoronaphthalene-2-sulfonamide. The title compound was obtained. LCMS (ESI) m/z: 480.1 [M+H]⁺.

### Example 279: 5-chloro-4-(cyclopentylmethoxy)-2-fluoro-N-((8-fluoroquinolin-5-yl)sulfonyl)benzamide

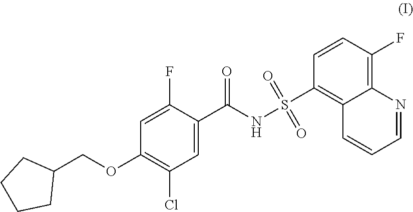 [SMILES: O=C(NS(=O)(=O)c1ccc(F)c2ncccc12)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzene-sulfonamide with 8-fluoroquinoline-5-sulfonamide. The title compound was obtained. ¹H NMR (400 MHz, DMSO-d₆) δ 12.93 (s, 1H), 9.16-9.05 (m, 2H), 8.50-8.38 (m, 1H), 7.92-7.79 (m, 2H), 7.64 (d, J=7.6 Hz, 1H), 7.19 (d, J=12.4 Hz, 1H), 3.99 (d, J=6.8 Hz, 2H), 2.35-2.25 (m, 1H), 1.81-1.67 (m, 2H), 1.65-1.48 (m, 4H), 1.27-1.40 (m, 2H). LCMS (ESI) m/z: 481.1 [M+H]⁺.

### Example 280: 5-chloro-N-((4-cyano-3-(trifluoromethyl)phenyl)sulfonyl)-4-(cyclopentylmethoxy)-2-fluorobenzamide

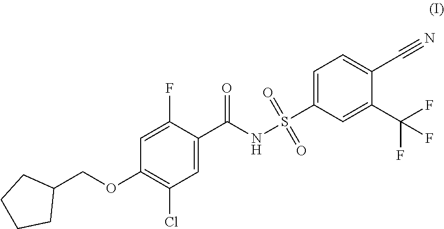 [SMILES: N#Cc1ccc(S(=O)(=O)NC(=O)c2cc(Cl)c(OCC3CCCC3)cc2F)cc1C(F)(F)F]

Following the procedure described in Example 204 and making non-critical variations as required to replace 4-(((1S,2S)-2-(dimethylamino)cyclohexyl)amino)-2,6-difluorobenzenesulfonamide with 4-cyano-3-(trifluoromethyl)benzenesulfonamide. The title compound was obtained. LCMS (ESI) m/z: 505.0 [M+H]⁺.

### Example 281: 5-chloro-4-(cyclopentylmethoxy)-N-((2,3-dihydrobenzo[b][1,4]dioxin-6-yl)sulfonyl)-2-fluorobenzamide

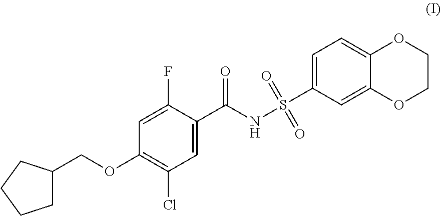 [SMILES: O=C(NS(=O)(=O)c1ccc2c(c1)OCCO2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 2,3-dihydro-1,4-benzodioxine-6-sulfonamide. The title compound was obtained. LCMS (ESI) m/z: 470.1 [M+H]⁺.

### Example 282: 5-chloro-4-(cyclopentylmethoxy)-2-fluoro-N-(indolin-6-ylsulfonyl)benzamide

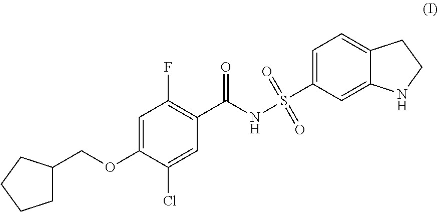 [SMILES: O=C(NS(=O)(=O)c1ccc2c(c1)NCC2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with indoline-6-sulfonamide. The title compound was obtained. LCMS (ESI) m/z: 453.1 [M+H]⁺.

### Example 283: 5-chloro-4-(cyclopentylmethoxy)-2-fluoro-N-((4-(trifluoromethyl)phenyl)sulfonyl)benzamide

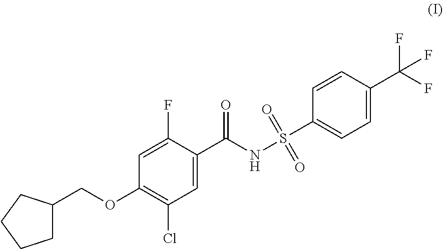 [SMILES: O=C(NS(=O)(=O)c1ccc(C(F)(F)F)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 4-(trifluoromethyl)benzenesulfonamide. The title compound was obtained. LCMS (ESI) m/z: 480.1 [M+H]⁺.

### Example 284: 5-chloro-4-(cyclopentylmethoxy)-2-fluoro-N-((trans-2-phenylcyclopropyl)sulfonyl)benzamide

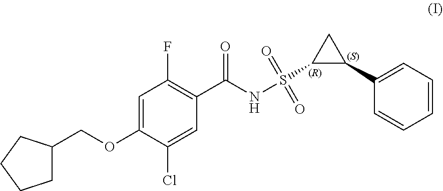

Following the procedure described in Example 204 and making non-critical variations as required to replace 4-(((1S,2S)-2-(dimethylamino)cyclohexyl)amino)-2,6-difluorobenzenesulfonamide with trans-2-phenylcyclopropane-1-sulfonamide. The title compound was obtained. LCMS (ESI) m/z: 452.1 [M+H]⁺.

### Example 285: 5-chloro-4-(cyclopentylmethoxy)-N-((6-ethoxypyridin-3-yl)sulfonyl)-2-fluorobenzamide

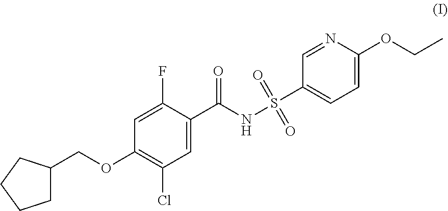 [SMILES: CCOc1ccc(S(=O)(=O)NC(=O)c2cc(Cl)c(OCC3CCCC3)cc2F)cn1]

Following the procedure described in Example 204 and making non-critical variations as required to replace 4-(((1S,2S)-2-(dimethylamino)cyclohexyl)amino)-2,6-difluorobenzenesulfonamide with 6-ethoxypyridine-3-sulfonamide. The title compound was obtained. LCMS (ESI) m/z: 457.1 [M+H]⁺.

### Example 286: 5-chloro-4-(cyclopentylmethoxy)-2-fluoro-N-((4-((1-methyl-1H-1,2,4-triazol-5-yl)methoxy)phenyl)sulfonyl)benzamide

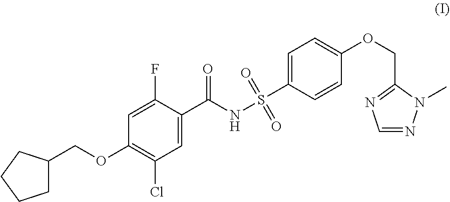 [SMILES: Cn1ncnc1COc1ccc(S(=O)(=O)NC(=O)c2cc(Cl)c(OCC3CCCC3)cc2F)cc1]

Following the procedure described in Example 204 and making non-critical variations as required to replace 4-(((1S,2S)-2-(dimethylamino)cyclohexyl)amino)-2,6-difluorobenzene-sulfonamide with 4-[(1-methyl-1H-1,2,4-triazol-5-yl)methoxy]benzene-1-sulfonamide. The title compound was obtained. ¹H NMR (400 MHz, DMSO-d₆) δ 12.37 (s, 1H), 7.99-7.89 (m, 3H), 7.67 (d, J=7.2 Hz, 1H), 7.30 (d, J=8.8 Hz, 2H), 7.21 (d, J=12.4 Hz, 1H), 5.44 (s, 2H), 4.01 (d, J=7.2 Hz, 2H), 3.91 (s, 3H), 2.46-2.28 (m, 1H), 1.82-1.70 (m, 2H), 1.68-1.46 (m, 4H), 1.38-1.28 (m, 2H). LCMS (ESI) m/z: 523.1 [M+H]⁺.

### Example 287: 5-chloro-4-(cyclopentylmethoxy)-2-fluoro-N-((4-(2,2,2-trifluoro-ethoxy)phenyl)sulfonyl)benzamide

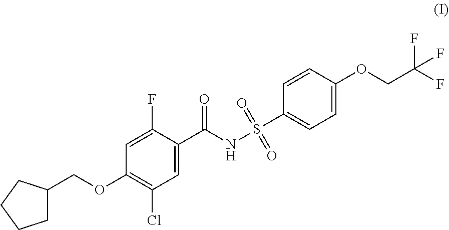 [SMILES: O=C(NS(=O)(=O)c1ccc(OCC(F)(F)F)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 4-(2,2,2-trifluoroethoxy)benzene-1-sulfonamide. The title compound was obtained. LCMS (ESI) m/z: 510.0 [M+H]⁺.

### Example 288: 5-chloro-N-((4-(4-chlorophenoxy)phenyl)sulfonyl)-4-(cyclopentylmethoxy)-2-fluorobenzamide

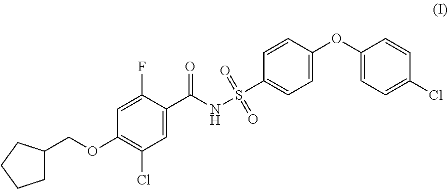 [SMILES: O=C(NS(=O)(=O)c1ccc(Oc2ccc(Cl)cc2)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 4-(4-chlorophenoxy)benzenesulfonamide. The title compound was obtained. LCMS (ESI) m/z: 538.0 [M+H]⁺.

### Example 289: 5-chloro-4-(cyclopentylmethoxy)-N-((3,4-dihydro-2H-benzo[b][1,4]dioxepin-7-yl)sulfonyl)-2-fluorobenzamide

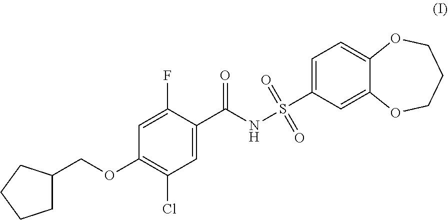 [SMILES: O=C(NS(=O)(=O)c1ccc2c(c1)OCCCO2)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 3,4-dihydro-2H-benzo[b][1,4]dioxepine-7-sulfonamide. The title compound was obtained. ¹H NMR (400 MHz, DMSO-d₆) δ 12.39 (s, 1H), 7.70 (d, J=7.6 Hz, 1H), 7.53-7.46 (m, 2H), 7.23 (d, J=12.8 Hz, 1H), 7.17 (d, J=8.4 Hz, 1H), 4.32-4.18 (m, 4H), 4.02 (d, J=6.8 Hz, 2H), 2.35-2.28 (m, 1H), 2.21-2.10 (m, 2H), 1.82-1.68 (m, 2H), 1.68-1.48 (m, 4H), 1.40-1.28 (m, 2H). LCMS (ESI) m/z: 484.1 [M+H]⁺.

### Example 290: 5-chloro-4-(cyclopentylmethoxy)-2-fluoro-N-(naphthalen-2-ylsulfonyl)benzamide

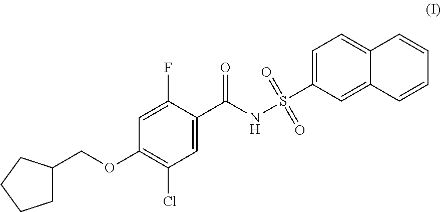 [SMILES: O=C(NS(=O)(=O)c1ccc2ccccc2c1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with naphthalene-2-sulfonamide. The title compound was obtained. LCMS (ESI) m/z: 462.1 [M+H]⁺.

### Example 291: 5-chloro-4-(cyclopentylmethoxy)-2-fluoro-N-((1-(2-methoxyphenyl)-1H-pyrazol-4-yl)sulfonyl)benzamide

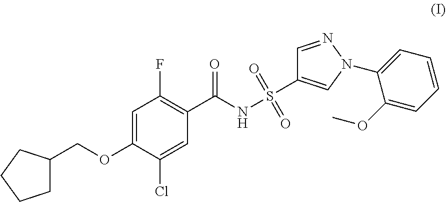 [SMILES: COc1ccccc1-n1cc(S(=O)(=O)NC(=O)c2cc(Cl)c(OCC3CCCC3)cc2F)cn1]

Following the procedure described in Example 204 and making non-critical variations as required to replace 4-(((1S,2S)-2-(dimethylamino)cyclohexyl)amino)-2,6-difluorobenzenesulfonamide with 1-(2-methoxyphenyl)-1H-pyrazole-4-sulfonamide. The title compound was obtained. ¹H NMR (400 MHz, DMSO-d₆) δ 12.50 (s, 1H), 8.78 (d, J=3.2 Hz, 1H), 8.18 (s, 1H), 7.74 (d, J=7.6 Hz, 1H), 7.68-7.60 (m, 1H), 7.49-7.40 (m, 1H), 7.33-7.26 (m, 1H), 7.23 (d, J=12.8 Hz, 1H), 7.13-7.06 (m, 1H), 4.02 (d, J=6.8 Hz, 2H), 3.89 (s, 3H), 2.39-2.23 (m, 1H), 1.82-1.69 (m, 2H), 1.67-1.46 (m, 4H), 1.40-1.26 (m, 2H). LCMS (ESI) m/z: 508.1 [M+H]⁺.

### Example 292: N-((4-((1H-1,2,4-triazol-1-yl)methyl)phenyl)sulfonyl)-5-chloro-4-(cyclopentylmethoxy)-2-fluorobenzamide

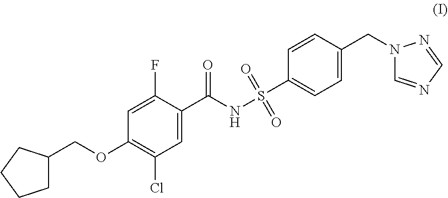 [SMILES: O=C(NS(=O)(=O)c1ccc(Cn2cncn2)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 4-[(1H-1,2,4-triazol-1-yl)methyl]benzene-1-sulfonamide. The title compound was obtained. LCMS (ESI) m/z: 493.1 [M+H]⁺.

### Example 293: 5-chloro-N-((2-chloro-4-(trifluoromethoxy)phenyl)sulfonyl)-4-(cyclopentylmethoxy)-2-fluorobenzamide

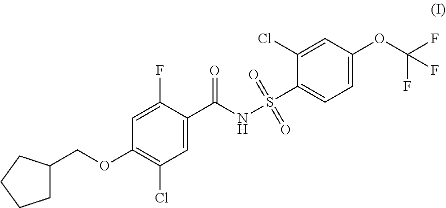 [SMILES: O=C(NS(=O)(=O)c1ccc(OC(F)(F)F)cc1Cl)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 2-chloro-4-(trifluoromethoxy)benzene-1-sulfonamide. The title compound was obtained. LCMS (ESI) m/z: 530.0 [M+H]⁺.

### Example 294: N-((4-(benzyloxy)phenyl)sulfonyl)-5-chloro-4-(cyclopentylmethoxy)-2-fluorobenzamide

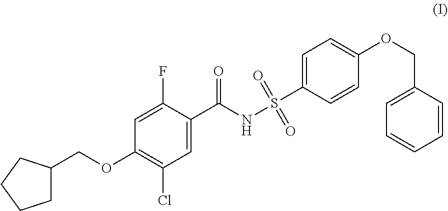 [SMILES: O=C(NS(=O)(=O)c1ccc(OCc2ccccc2)cc1)c1cc(Cl)c(OCC2CCCC2)cc1F]

Following the procedure described in Example 204 and making non-critical variations as required to replace 4-(((1S,2S)-2-(dimethylamino)cyclohexyl)amino)-2,6-difluorobenzenesulfonamide with 4-(benzyloxy)benzenesulfonamide. The title compound was obtained. LCMS (ESI) m/z: 518.1.1 [M+H]⁺.

### Example 295: 5-chloro-4-(cyclopentylmethoxy)-2-fluoro-N-((4-(pyrimidin-2-ylmethoxy)phenyl)sulfonyl)benzamide

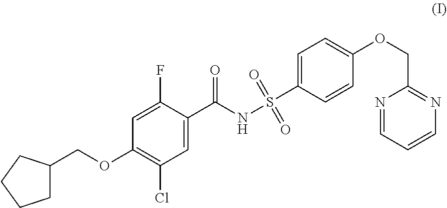 [SMILES: O=C(NS(=O)(=O)c1ccc(OCc2ncccn2)cc1)c1cc(Cl)c(OCC2CCCC2)cc1F]

### Step 1: 4-[(pyrimidin-2-yl)methoxy]benzene-1-sulfonamide

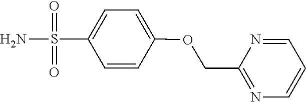 [SMILES: NS(=O)(=O)c1ccc(OCc2ncccn2)cc1]

To a solution of 4-hydroxybenzenesulfonamide (39 mg, 0.23 mmol) and 2-(chloromethyl)pyrimidine (58 mg, 0.45 mmol) in DMF (1 mL) was added K₂CO₃ (95 mg, 0.69 mmol). The mixture was stirred at 25° C. for 16 h. The reaction was filtered and concentrated in vacuo to afford the title compound (30 mg, crude) that required no further purification. LCMS (ESI) m/z: 266.1 [M+H]⁺.

### Step 2: 5-chloro-4-(cyclopentylmethoxy)-2-fluoro-N-((4-(pyrimidin-2-ylmethoxy)phenyl)sulfonyl)benzamide

To a solution of 5-chloro-4-(cyclopentylmethoxy)-2-fluorobenzoic acid (27 mg, 0.10 mmol) in DCM (1 mL) was added EDCI (23 mg, 0.12 mmol). The mixture was stirred at 30° C. for 2 h before the addition of 4-[(pyrimidin-2-yl)methoxy]benzene-1-sulfonamide (30 mg, 0.12 mmol) and DMAP (18 mg, 0.15 mmol). The reaction was then stirred at 50° C. for 16 h. After cooling to room temperature, the reaction was concentrated in vacuo and the crude residue was purified by reverse phase chromatography (acetonitrile 30-70%/0.2% formic acid in water) to afford the title compound (5 mg, 9%). LCMS (ESI) m/z: 520.1 [M+H]⁺.

### Example 296: N-((4-(azetidin-3-ylmethoxy)phenyl)sulfonyl)-5-chloro-4-(cyclopentylmethoxy)-2-fluorobenzamide

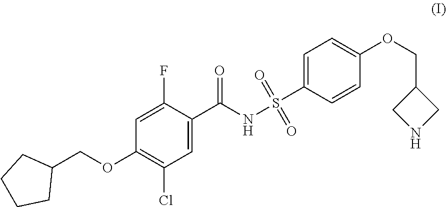 [SMILES: O=C(NS(=O)(=O)c1ccc(OCC2CNC2)cc1)c1cc(Cl)c(OCC2CCCC2)cc1F]

### Step 1: tert-butyl 3-(chloromethyl)azetidine-1-carboxylate

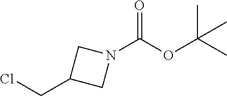

To a solution of tert-butyl 3-(hydroxymethyl)azetidine-1-carboxylate (93.5 mg, 0.50 mmol) in DCM (2.5 mL) was added SOCl₂ (0.5 mL) at 0° C. The mixture was stirred at 50° C. for 2 h. The reaction was concentrated in vacuo to afford the title compound (82.0 mg, crude) that required no further purification. LCMS (ESI) m/z: 206.09 [M+H]⁺.

### Step 2: tert-butyl 3-((4-sulfamoylphenoxy)methyl)azetidine-1-carboxylate

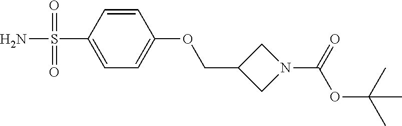 [SMILES: CC(C)(C)OC(=O)N1CC(COc2ccc(S(N)(=O)=O)cc2)C1]

To a solution of 4-hydroxybenzenesulfonamide (52 mg, 0.30 mmol) and tert-butyl 3-(chloromethyl)azetidine-1-carboxylate (82 mg, 0.40 mmol) in DMF (2 mL) was added Cs₂CO₃ (293 mg, 0.90 mmol). The mixture was stirred at 50° C. for 16 h. After cooling to room temperature, the reaction was filtered and the filtrate was concentrated in vacuo to afford the title compound (86 mg, crude) that required no further purification. LCMS (ESI) m/z: 343.1 [M+H]⁺.

### Step 3: tert-butyl 3-((4-(N-(5-chloro-4-(cyclopentylmethoxy)-2-fluorobenzoyl)sulfamoyl)phenoxy)methyl)azetidine-1-carboxylate

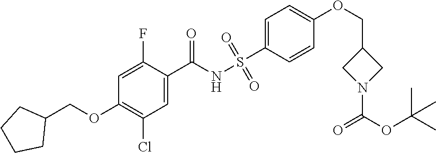 [SMILES: CC(C)(C)OC(=O)N1CC(COc2ccc(S(=O)(=O)NC(=O)c3cc(Cl)c(OCC4CCCC4)cc3F)cc2)C1]

To a solution of 5-chloro-4-(cyclopentylmethoxy)-2-fluorobenzoic acid (54 mg, 0.20 mmol) in DCM (1 mL) was added EDCI (46 mg, 0.24 mmol). The mixture was stirred at 30° C. for 2 h before the addition of tert-butyl 3-((4-sulfamoylphenoxy)methyl)azetidine-1-carboxylate (86 mg, 0.25 mmol) and DMAP (37 mg, 0.30 mmol). The reaction was then stirred at 50° C. for 16 h. After cooling to room temperature, the reaction was filtered and the filtrate was concentrated in vacuo to afford the title compound (89 mg, crude) that required no further purification. LCMS (ESI) m/z: 597.2 [M+H]⁺.

### Step 4: N-((4-(azetidin-3-ylmethoxy)phenyl)sulfonyl)-5-chloro-4-(cyclopentylmethoxy)-2-fluorobenzamide

To a solution of tert-butyl 3-((4-(N-(5-chloro-4-(cyclopentylmethoxy)-2-fluorobenzoyl)sulfamoyl)phenoxy)methyl)azetidine-1-carboxylate (89 mg, 0.15 mmol) in DCM (0.9 mL) was added TFA (0.3 mL). The mixture was stirred at 30° C. for 3 h. The mixture was concentrated in vacuo and the crude residue was purified by reverse phase chromatography (acetonitrile 15-45%/0.2% formic acid in water) to afford the title compound (3.0 mg, 6%). LCMS (ESI) m/z: 497.1 [M+H]⁺.

### Example 297: N-((4-((2-acetyl-1,2,3,4-tetrahydroisoquinolin-8-yl)methoxy)phenyl)sulfonyl)-5-chloro-4-(cyclopentylmethoxy)-2-fluorobenzamide

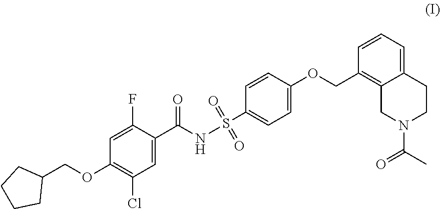 [SMILES: CC(=O)N1CCc2cccc(COc3ccc(S(=O)(=O)NC(=O)c4cc(Cl)c(OCC5CCCC5)cc4F)cc3)c2C1]

Following the procedure described in Example 296 and making non-critical variations as required to replace tert-butyl 3-(hydroxymethyl)azetidine-1-carboxylate with 1-(8-(hydroxymethyl)-3,4-dihydroisoquinolin-2(1H)-yl)ethanone. The title compound was obtained. LCMS (ESI) m/z: 615.1 [M+H]⁺.

### Example 298: 5-chloro-4-(cyclopentylmethoxy)-2-fluoro-N-((4-((3-fluorobenzyl)oxy)-phenyl)sulfonyl)benzamide

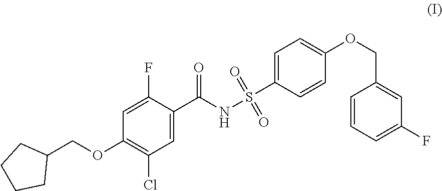 [SMILES: O=C(NS(=O)(=O)c1ccc(OCc2cccc(F)c2)cc1)c1cc(Cl)c(OCC2CCCC2)cc1F]

Following the procedure described in Example 295 and making non-critical variations as required to replace 2-(chloromethyl)pyrimidine with 1-(chloromethyl)-3-fluorobenzene. The title compound was obtained. LCMS (ESI) m/z: 536.1 [M+H]⁺.

### Example 299: 5-chloro-4-(cyclopentylmethoxy)-2-fluoro-N-((4-((1-methyl-1H-pyrazol-4-yl)methoxy)phenyl)sulfonyl)benzamide

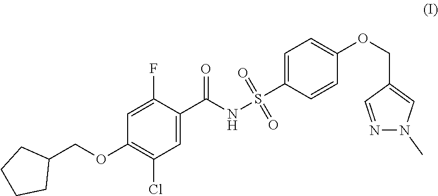 [SMILES: Cn1cc(COc2ccc(S(=O)(=O)NC(=O)c3cc(Cl)c(OCC4CCCC4)cc3F)cc2)cn1]

Following the procedure described in Example 296 and making non-critical variations as required to replace tert-butyl 3-(hydroxymethyl)azetidine-1-carboxylate with (1-methyl-1H-pyrazol-4-yl)methanol. The title compound was obtained. LCMS (ESI) m/z: 522.1 [M+H]⁺.

### Example 300: N-((4-((3-(aminomethyl)benzyl)oxy)phenyl)sulfonyl)-5-chloro-4-(cyclopentylmethoxy)-2-fluorobenzamide

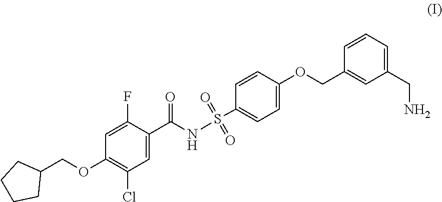 [SMILES: NCc1cccc(COc2ccc(S(=O)(=O)NC(=O)c3cc(Cl)c(OCC4CCCC4)cc3F)cc2)c1]

Following the procedure described in Example 296 and making non-critical variations as required to replace tert-butyl 3-(hydroxymethyl)azetidine-1-carboxylate with tert-butyl 3-(hydroxymethyl)benzylcarbamate. The title compound was obtained. ¹H NMR (400 MHz, DMSO-d₆) δ 8.15 (s, 2H), 7.77-7.70 (m, 3H), 7.60-7.36 (m, 4H), 7.00 (d, J=8.8 Hz, 2H), 6.92 (d, J=12.4 Hz, 1H), 5.17 (s, 2H), 4.06 (s, 2H), 3.94 (d, J=6.8 Hz, 2H), 2.36-2.28 (m, 1H), 1.80-1.68 (m, 2H), 1.67-1.47 (m, 4H), 1.40-1.30 (m, 2H). LCMS (ESI) m/z: 547.1 [M+H]⁺.

### Example 301: 5-chloro-4-(cyclopentylmethoxy)-2-fluoro-N-((4-(pyridin-2-ylmethoxy)phenyl)sulfonyl)benzamide

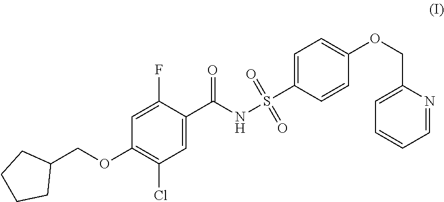 [SMILES: O=C(NS(=O)(=O)c1ccc(OCc2ccccn2)cc1)c1cc(Cl)c(OCC2CCCC2)cc1F]

Following the procedure described in Example 96 and making non-critical variations as required to replace tert-butyl 3-(hydroxymethyl)azetidine-1-carboxylate with pyridin-2-ylmethanol. The title compound was obtained. LCMS (ESI) m/z: 519.1 [M+H]⁺.

### Example 302: (R)-5-chloro-4-(cyclopentylmethoxy)-2-fluoro-N-((4-(pyrrolidin-3-ylmethoxy)phenyl)sulfonyl)benzamide

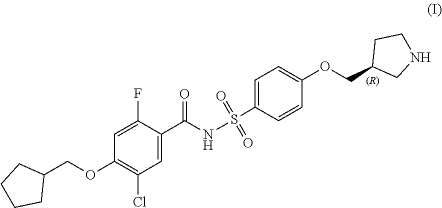

Following the procedure described in Example 296 and making non-critical variations as required to replace tert-butyl 3-(hydroxymethyl)azetidine-1-carboxylate with tert-butyl (R)-3-(hydroxymethyl)pyrrolidine-1-carboxylate. The title compound was obtained. LCMS (ESI) m/z: 511.1 [M+H]⁺.

### Example 303: 5-chloro-4-(cyclopentylmethoxy)-N-((4-((3,5-dimethyl-benzyl)oxy)phenyl)sulfonyl)-2-fluorobenzamide

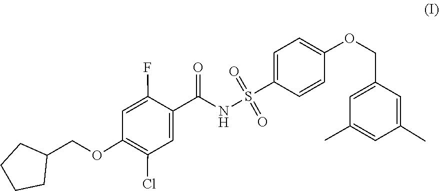 [SMILES: Cc1cc(C)cc(COc2ccc(S(=O)(=O)NC(=O)c3cc(Cl)c(OCC4CCCC4)cc3F)cc2)c1]

Following the procedure described in Example 295 and making non-critical variations as required to replace 2-(chloromethyl)pyrimidine with 1-(chloromethyl)-3,5-dimethylbenzene. The title compound was obtained. LCMS (ESI) m/z: 546.1 [M+H]⁺.

### Example 304: 5-chloro-4-(cyclopentylmethoxy)-2-fluoro-N-((4-(pyrimidin-5-ylmethoxy)phenyl)sulfonyl)benzamide

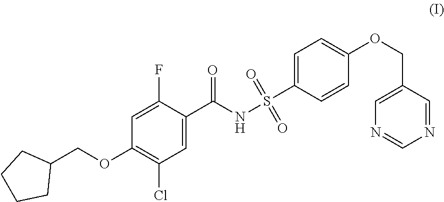 [SMILES: O=C(NS(=O)(=O)c1ccc(OCc2cncnc2)cc1)c1cc(Cl)c(OCC2CCCC2)cc1F]

Following the procedure described in Example 295 and making non-critical variations as required to replace tert-butyl 3-(hydroxymethyl)azetidine-1-carboxylate with pyrimidin-5-ylmethanol. The title compound was obtained. LCMS (ESI) m/z: 520.0 [M+H]⁺.

### Example 305: 5-chloro-4-(cyclopentylmethoxy)-2-fluoro-N-((4-(pyrrolidin-2-ylmethoxy)phenyl)sulfonyl)benzamide

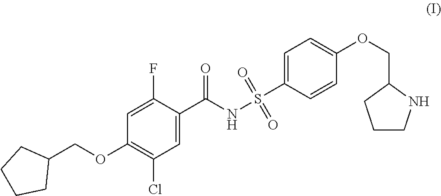 [SMILES: O=C(NS(=O)(=O)c1ccc(OCC2CCCN2)cc1)c1cc(Cl)c(OCC2CCCC2)cc1F]

Following the procedure described in Example 296 and making non-critical variations as required to replace tert-butyl 3-(hydroxymethyl)azetidine-1-carboxylate with tert-butyl 2-(hydroxymethyl)pyrrolidine-1-carboxylate. The title compound was obtained. LCMS (ESI) m/z: 511.1 [M+H]⁺.

### Example 306: 5-chloro-4-(cyclopentylmethoxy)-N-((4-((3,5-dimethoxy-benzyl)oxy)phenyl)sulfonyl)-2-fluorobenzamide

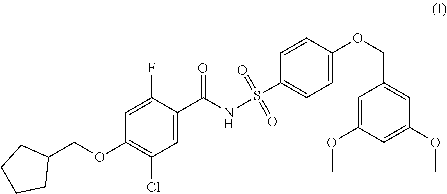 [SMILES: COc1cc(COc2ccc(S(=O)(=O)NC(=O)c3cc(Cl)c(OCC4CCCC4)cc3F)cc2)cc(OC)c1]

Following the procedure described in Example 295 and making non-critical variations as required to replace 2-(chloromethyl)pyrimidine with 1-(chloromethyl)-3,5-dimethoxybenzene. The title compound was obtained. LCMS (ESI) m/z: 578.1 [M+H]⁺.

### Example 307: 5-chloro-4-(cyclopentylmethoxy)-N-((4-((3,5-difluorobenzyl)oxy)phenyl)sulfonyl)-2-fluorobenzamide

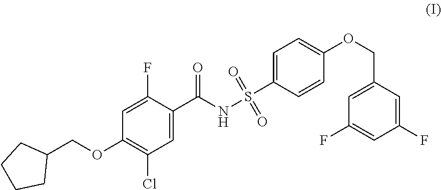 [SMILES: O=C(NS(=O)(=O)c1ccc(OCc2cc(F)cc(F)c2)cc1)c1cc(Cl)c(OCC2CCCC2)cc1F]

Following the procedure described in Example 295 and making non-critical variations as required to replace 2-(chloromethyl)pyrimidine with 1-(chloromethyl)-3,5-difluorobenzene. The title compound was obtained. ¹H NMR (400 MHz, DMSO-d₆) δ 12.37 (s, 1H), 7.91 (d, J=8.8 Hz, 2H), 7.67 (d, J=7.6 Hz, 1H), 7.26-7.15 (m, 6H), 5.24 (s, 2H), 4.00 (d, J=6.8 Hz, 2H), 2.38-2.22 (m, 1H), 1.81-1.70 (m, 2H), 1.68-1.42 (m, 4H), 1.39-1.28 (m, 2H). LCMS (ESI) m/z: 554.1 [M+H]⁺.

### Example 308: 5-chloro-4-(cyclopentylmethoxy)-2-fluoro-N-((4-((3-methoxybenzyl)oxy)phenyl)sulfonyl)benzamide

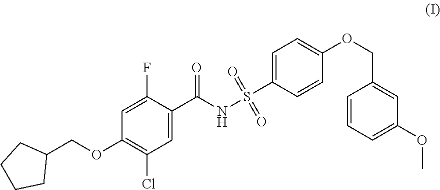 [SMILES: COc1cccc(COc2ccc(S(=O)(=O)NC(=O)c3cc(Cl)c(OCC4CCCC4)cc3F)cc2)c1]

Following the procedure described in Example 295 and making non-critical variations as required to replace 2-(chloromethyl)pyrimidine with 1-(chloromethyl)-3-methoxybenzene. The title compound was obtained. LCMS (ESI) m/z: 548.1 [M+H]⁺.

### Example 309: 5-chloro-4-(cyclopentylmethoxy)-2-fluoro-N-((4-((4-methoxybenzyl)oxy)phenyl)sulfonyl)benzamide

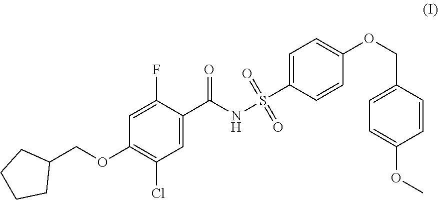 [SMILES: COc1ccc(COc2ccc(S(=O)(=O)NC(=O)c3cc(Cl)c(OCC4CCCC4)cc3F)cc2)cc1]

Following the procedure described in Example 295 and making non-critical variations as required to replace 2-(chloromethyl)pyrimidine with 1-(chloromethyl)-4-methoxybenzene. The title compound was obtained. LCMS (ESI) m/z: 548.1 [M+H]⁺.

### Example 310: 5-chloro-4-(cyclopentylmethoxy)-2-fluoro-N-((4-(pyridin-3-ylmethoxy)phenyl)sulfonyl)benzamide

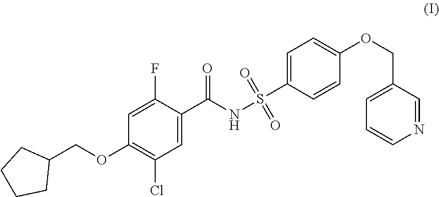 [SMILES: O=C(NS(=O)(=O)c1ccc(OCc2cccnc2)cc1)c1cc(Cl)c(OCC2CCCC2)cc1F]

Following the procedure described in Example 296 and making non-critical variations as required to replace tert-butyl 3-(hydroxymethyl)azetidine-1-carboxylate with pyridin-3-ylmethanol. The title compound was obtained. LCMS (ESI) m/z: 519.1 [M+H]⁺.

### Example 311: 5-chloro-4-(cyclopentylmethoxy)-2-fluoro-N-((4-((3-(morpholinomethyl)benzyl)oxy)phenyl)sulfonyl)benzamide

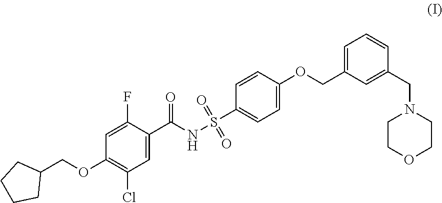 [SMILES: O=C(NS(=O)(=O)c1ccc(OCc2cccc(CN3CCOCC3)c2)cc1)c1cc(Cl)c(OCC2CCCC2)cc1F]

Following the procedure described in Example 296 and making non-critical variations as required to replace tert-butyl 3-(hydroxymethyl)azetidine-1-carboxylate with (3-(morpholinomethyl)phenyl)methanol. The title compound was obtained. ¹H NMR (400 MHz, DMSO-d₆) δ 11.36 (s, 1H), 7.84 (d, J=8.8 Hz, 2H), 7.69 (d, J=8.0 Hz, 1H), 7.49-7.33 (m, 4H), 7.22-7.05 (m, 3H), 5.21 (s, 2H), 3.98 (d, J=6.8 Hz, 2H), 3.85 (s, 2H), 3.68-3.59 (m, 4H), 2.80-2.56 (m, 4H), 2.38-2.26 (m, 1H), 1.81-1.72 (m, 2H), 1.65-1.48 (m, 4H), 1.39-1.29 (m, 2H). LCMS (ESI) m/z: 617.1 [M+H]⁺.

### Example 312: 5-chloro-4-(cyclopentylmethoxy)-2-fluoro-N-((4-((1-methyl-1H-imidazol-2-yl)methoxy)phenyl)sulfonyl)benzamide

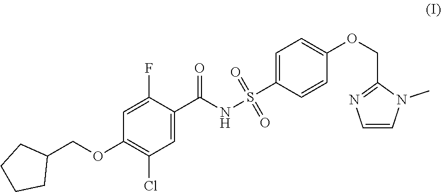 [SMILES: Cn1ccnc1COc1ccc(S(=O)(=O)NC(=O)c2cc(Cl)c(OCC3CCCC3)cc2F)cc1]

Following the procedure described in Example 295 and making non-critical variations as required to replace 2-(chloromethyl)pyrimidine with 2-(chloromethyl)-1-methyl-1H-imidazole. The title compound was obtained. LCMS (ESI) m/z: 522.1 [M+H]⁺.

### Example 313: 5-chloro-4-(cyclopentylmethoxy)-2-fluoro-N-((4-((4-methylbenzyl)oxy)phenyl)sulfonyl)benzamide

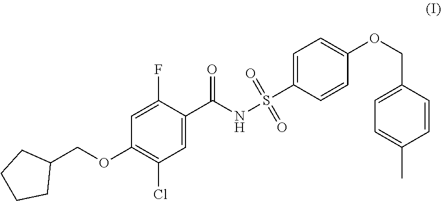 [SMILES: Cc1ccc(COc2ccc(S(=O)(=O)NC(=O)c3cc(Cl)c(OCC4CCCC4)cc3F)cc2)cc1]

Following the procedure described in Example 295 and making non-critical variations as required to replace 2-(chloromethyl)pyrimidine with 1-(chloromethyl)-4-methylbenzene. The title compound was obtained. LCMS (ESI) m/z: 532.1 [M+H]⁺.

### Example 314: N-((4-(3-aminopropoxy)phenyl)sulfonyl)-5-chloro-4-(cyclopentylmethoxy)-2-fluorobenzamide

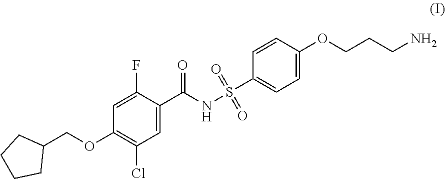 [SMILES: NCCCOc1ccc(S(=O)(=O)NC(=O)c2cc(Cl)c(OCC3CCCC3)cc2F)cc1]

Following the procedure described in Example 296 and making non-critical variations as required to replace tert-butyl 3-(hydroxymethyl)azetidine-1-carboxylate with tert-butyl (3-hydroxypropyl)carbamate. The title compound was obtained. LCMS (ESI) m/z: 485.1 [M+H]⁺.

### Example 315: 5-chloro-4-(cyclopentylmethoxy)-2-fluoro-N-((4-((1-methyl-1H-pyrazol-5-yl)methoxy)phenyl)sulfonyl)benzamide

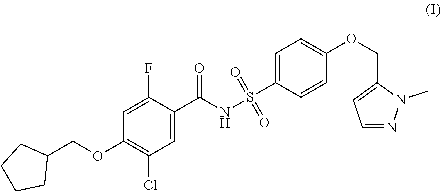 [SMILES: Cn1nccc1COc1ccc(S(=O)(=O)NC(=O)c2cc(Cl)c(OCC3CCCC3)cc2F)cc1]

Following the procedure described in Example 296 and making non-critical variations as required to replace tert-butyl 3-(hydroxymethyl)azetidine-1-carboxylate with (1-methyl-1H-pyrazol-5-yl)methanol. The title compound was obtained. LCMS (ESI) m/z: 522.1 [M+H]⁺.

### Example 316: 5-chloro-4-(cyclopentylmethoxy)-2-fluoro-N-((4-((1-methyl-1H-imidazol-5-yl)methoxy)phenyl)sulfonyl)benzamide

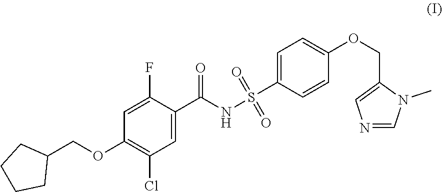 [SMILES: Cn1cncc1COc1ccc(S(=O)(=O)NC(=O)c2cc(Cl)c(OCC3CCCC3)cc2F)cc1]

Following the procedure described in Example 296 and making non-critical variations as required to replace tert-butyl 3-(hydroxymethyl)azetidine-1-carboxylate with (1-methyl-1H-imidazol-5-yl)methanol. The title compound was obtained. LCMS (ESI) m/z: 522.1 [M+H]⁺.

### Example 317: 5-chloro-4-(cyclopentylmethoxy)-N-((4-((2,4-difluorobenzyl)oxy)phenyl)sulfonyl)-2-fluorobenzamide

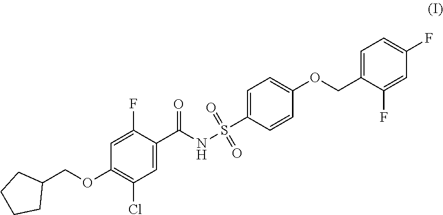 [SMILES: O=C(NS(=O)(=O)c1ccc(OCc2ccc(F)cc2F)cc1)c1cc(Cl)c(OCC2CCCC2)cc1F]

Following the procedure described in Example 295 and making non-critical variations as required to replace 2-(chloromethyl)pyrimidine with 1-(chloromethyl)-2,4-difluorobenzene. The title compound was obtained. LCMS (ESI) m/z: 554.1 [M+H]⁺.

### Example 318: 5-chloro-4-(cyclopentylmethoxy)-2-fluoro-N-((4-((2-methoxybenzyl)oxy)phenyl)sulfonyl)benzamide

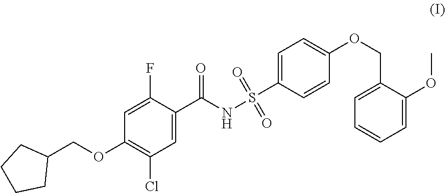 [SMILES: COc1ccccc1COc1ccc(S(=O)(=O)NC(=O)c2cc(Cl)c(OCC3CCCC3)cc2F)cc1]

Following the procedure described in Example 295 and making non-critical variations as required to replace 2-(chloromethyl)pyrimidine with 1-(chloromethyl)-2-methoxybenzene. The title compound was obtained. LCMS (ESI) m/z: 548.1 [M+H]⁺.

### Example 319: 5-chloro-4-(cyclopentylmethoxy)-2-fluoro-N-((4-((4-fluorobenzyl)oxy)phenyl)sulfonyl)benzamide

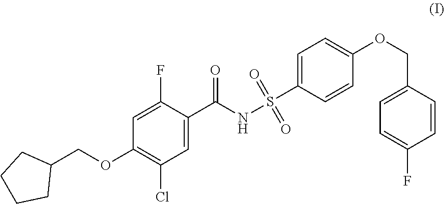 [SMILES: O=C(NS(=O)(=O)c1ccc(OCc2ccc(F)cc2)cc1)c1cc(Cl)c(OCC2CCCC2)cc1F]

Following the procedure described in Example 295 and making non-critical variations as required to replace 2-(chloromethyl)pyrimidine with 1-(chloromethyl)-4-fluorobenzene. The title compound was obtained. LCMS (ESI) m/z: 536.1 [M+H]⁺.

### Example 320: 4-(cyclopentylmethoxy)-5-cyclopropyl-N-((4-(((1S,2S)-2-(dimethylamino)cyclohexyl)amino)-2-fluorophenyl)sulfonyl)-2-fluorobenzamide

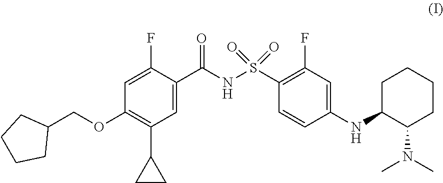 [SMILES: CN(C)[C@H]1CCCC[C@@H]1Nc1ccc(S(=O)(=O)NC(=O)c2cc(C3CC3)c(OCC3CCCC3)cc2F)c(F)c1]

Following the procedure described in compound 206 and replacing 5-chloro-4-(cyclopentylmethoxy)-2-fluorobenzoic acid with 4-(cyclopentylmethoxy)-5-cyclopropyl-2-fluorobenzoic acid, the title compound was obtained as a white solid.

¹H NMR (400 MHz, DMSO-d₆) δ 7.50 (t, J=8.8 Hz, 1H), 7.20 (d, J=8.8 Hz, 1H), 6.65 (d, J=12.0 Hz, 1H), 6.52-6.34 (m, 2H), 5.99 (s, 1H), 3.87 (d, J=6.8 Hz, 2H), 3.72-3.52 (m, 1H), 3.05-2.81 (m, 1H), 2.61-2.52 (m, 6H), 2.37-2.26 (m, 1H), 2.07-1.90 (m, 3H), 1.87-1.71 (m, 3H), 1.66-1.52 (m, 5H), 1.45-1.30 (m, 4H), 1.29-1.20 (m, 1H), 1.15-1.05 (m, 1H), 0.91-0.80 (m, 2H), 0.60-0.45 (m, 2H). HRMS m z calcd for C₃₀H₃₉F₂N₃O₄S [M+H]⁺: 576.2702. Found 576.2713.

The compounds of Examples 321-333 were prepared according to the methods developed and deployed for other compounds herein.

### Example 321: 5-chloro-4-(cyclopentylmethoxy)-2-fluoro-N-((octahydro-2,7-naphthyridin-2(1H)-yl)sulfonyl)benzamide

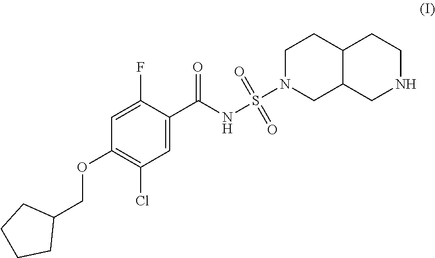 [SMILES: O=C(NS(=O)(=O)N1CCC2CCNCC2C1)c1cc(Cl)c(OCC2CCCC2)cc1F]

### Example 322: 5-chloro-4-(cyclopentylmethoxy)-2-fluoro-N-(pyrrolidin-3-ylsulfonyl)-benzamide

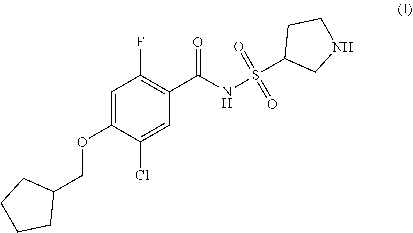 [SMILES: O=C(NS(=O)(=O)C1CCNC1)c1cc(Cl)c(OCC2CCCC2)cc1F]

### Example 323: 5-chloro-4-(cyclopentylmethoxy)-2-fluoro-N-((1-methylpyrrolidin-3-yl)sulfonyl)benzamide

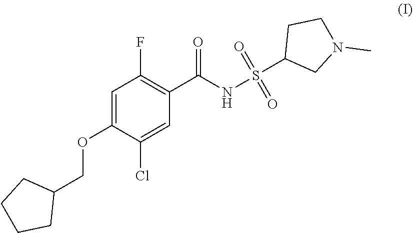 [SMILES: CN1CCC(S(=O)(=O)NC(=O)c2cc(Cl)c(OCC3CCCC3)cc2F)C1]

### Example 324: 5-chloro-4-(cyclopentylmethoxy)-2-fluoro-N-(piperidin-4-ylsulfonyl)-benzamide

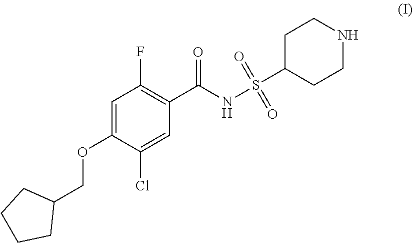 [SMILES: O=C(NS(=O)(=O)C1CCNCC1)c1cc(Cl)c(OCC2CCCC2)cc1F]

### Example 325: 5-chloro-4-(cyclopentylmethoxy)-2-fluoro-N-((4-(pyrrolidin-3-yloxy)phenyl)sulfonyl)benzamide

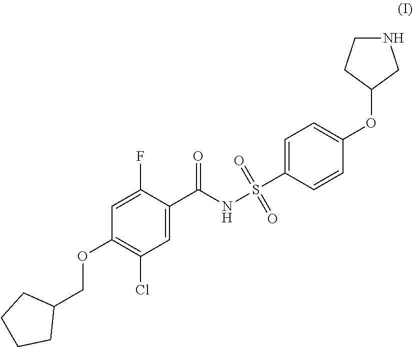 [SMILES: O=C(NS(=O)(=O)c1ccc(OC2CCNC2)cc1)c1cc(Cl)c(OCC2CCCC2)cc1F]

### Example 326: (S)-5-chloro-4-(cyclopentylmethoxy)-2-fluoro-N-((4-((1-methylpyrrolidin-3-yl)oxy)piperidin-1-yl)sulfonyl)benzamide

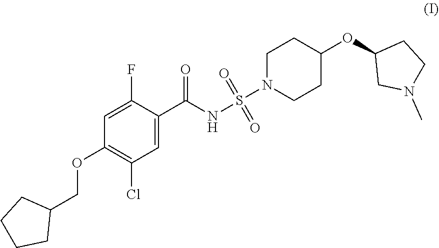 [SMILES: CN1CC[C@H](OC2CCN(S(=O)(=O)NC(=O)c3cc(Cl)c(OCC4CCCC4)cc3F)CC2)C1]

### Example 327: tert-butyl 6-((N-(5-chloro-4-(cyclopentylmethoxy)-2-fluorobenzoyl)-sulfamoyl)amino)bicyclo[3.1.0]hexane-3-carboxylate

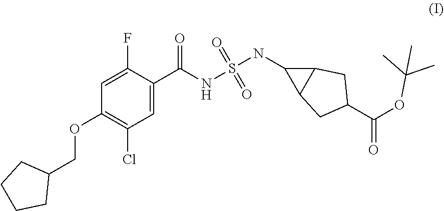 [SMILES: CC(C)(C)OC(=O)C1CC2C(C1)C2NS(=O)(=O)NC(=O)c1cc(Cl)c(OCC2CCCC2)cc1F]

### Example 328: 5-chloro-4-(cyclopentylmethoxy)-N-(cyclopropylsulfonyl)-2-fluorobenzamide

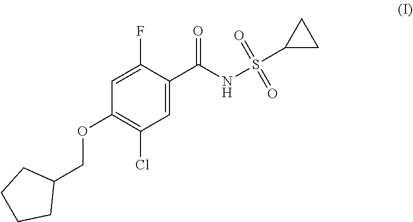 [SMILES: O=C(NS(=O)(=O)C1CC1)c1cc(Cl)c(OCC2CCCC2)cc1F]

### Example 329: 5-chloro-4-(cyclopentylmethoxy)-N-((4-(dimethylamino)piperidin-1-yl)sulfonyl)-2-fluorobenzamide

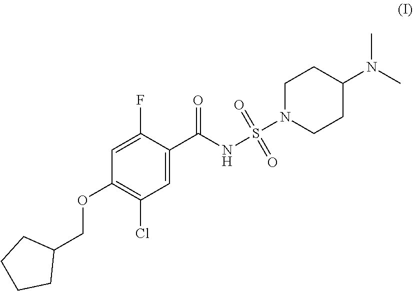 [SMILES: CN(C)C1CCN(S(=O)(=O)NC(=O)c2cc(Cl)c(OCC3CCCC3)cc2F)CC1]

### Example 330: N-((2-(aminomethyl)morpholino)sulfonyl)-4-(cyclopentylmethoxy)-5-cyclopropyl-2-fluorobenzamide

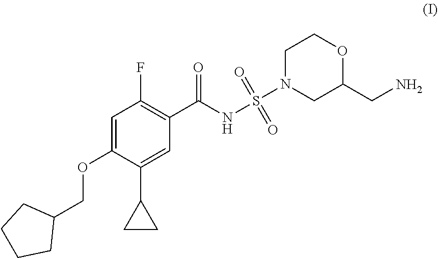 [SMILES: NCC1CN(S(=O)(=O)NC(=O)c2cc(C3CC3)c(OCC3CCCC3)cc2F)CCO1]

### Example 331: 4-(cyclopentylmethoxy)-5-cyclopropyl-N-((4-(((1S,2S)-2-(dimethylamino)cyclohexyl)oxy)piperidin-1-yl)sulfonyl)-2-fluorobenzamide

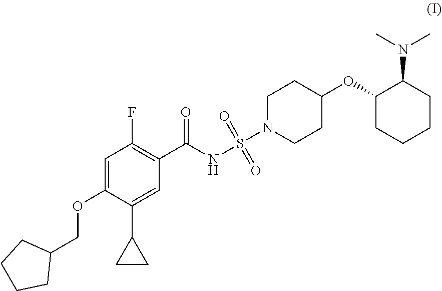 [SMILES: CN(C)[C@H]1CCCC[C@@H]1OC1CCN(S(=O)(=O)NC(=O)c2cc(C3CC3)c(OCC3CCCC3)cc2F)CC1]

### Example 332: 4-(((1S,2S)-2-(dimethylamino)cyclohexyl)amino)-2-fluoro-N-(thiazol-2-yl)benzenesulfonamide

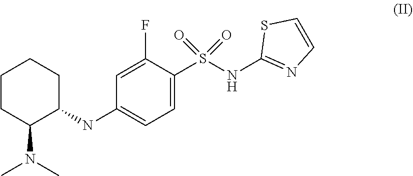 [SMILES: CN(C)[C@H]1CCCC[C@@H]1Nc1ccc(S(=O)(=O)Nc2nccs2)c(F)c1]

### Example 333: N-(7-chloro-6-(cyclopentylmethoxy)benzo[d]thiazol-2-yl)-4-(((1S,2S)-2-(dimethylamino)cyclohexyl)amino)-2-fluorobenzenesulfonamide

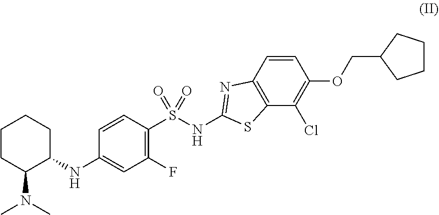 [SMILES: CN(C)[C@H]1CCCC[C@@H]1Nc1ccc(S(=O)(=O)Nc2nc3ccc(OCC4CCCC4)c(Cl)c3s2)c(F)c1]

### Example 400: Electrophysiological Assay (EP) (in vitro assay)

Patch voltage clamp electrophysiology allows for the direct measurement and quantification of block of voltage-gated sodium channels (NaV's), and allows the determination of the time- and voltage-dependence of block which has been interpreted as differential binding to the resting, open, and inactivated states of the sodium channel (Hille, B., Journal of General Physiology (1977), 69: 497-515).

The following voltage clamp electrophysiology studies are performed on representative compounds using cells heterologously expressing Nav1.7 or Nav1.5 channels. cDNAs for Nav1.7 (NM_002977) and Nav1.5 (AC137587) are stably expressed in Chinese Hamstr Ovary (CHO) cells and CHL (Chinese Hamster Lung) cells respectively. Sodium currents are measured in the whole-cell configuration using Syncropatch 384PE (Nanlon Technologies, Germany). 1NPC®-384 chips with custom medium resistance and single hole mode are used. Internal solution consists of (in mM): 110 CsCl, 10 CsCl, 20 EGTA, and 10 Hepes (pH adjusted to 7.2); and external solution contains (in mM): 60 NMDG, 80 NaCl, 4 KCl, 1 MgCl₂, 2 CaCl₂), 2 D-Glucose monohydrate, 10 Hepes (pH adjusted to 7.4 with NaOH).

After system flushing, testing compounds are dissolved in external solution containing 0.1% Pluronic F-127. The chip is moved into the measuring head and the instrument primes the chip with external and internal solutions. 10 μl cells are added to the chip from a cell hotel, and a negative pressure of −50 mBar is applied to form a seal. Following treatment with seal enhancer solution and wash-off with external solution, negative pressure of −250 mbar is applied for 1 second to achieve the whole-cell configuration, followed by three washing steps in external solution, 20 μl of compounds is added to 40 μl in each well (1:3 dilution of compounds), and after mixing, 20 μl is removed so the volume is retained at 40 ul. After approximately 13 minutes recordings, 20 μl/well of 2 μM TTX, or 333 μM Tetracaine (for Nav1.5) is added to achieve full block.

For voltage protocol, an holding potential of −50 mV is applied during the whole experiment. A depolarizing step is applied to −10 mV for 10 ms, followed by a hyperpolarization step to −150 mV for 20 ms to allow channel recovery from inactivation. A second depolarizing step is applied from −150 mV to −10 mV for 10 ms, where currents are measured to derive blocking effects of compounds. Inhibition is determined based on 7.5 min. of compound incubation.

Data for representative compounds is provided in Table 1. Compounds in the following table that are not prepared in the Examples above were prepared using synthetic schemes and reagents similar to those used in the Examples.

TABLE 1

| | | | hNaV1.7 |
|---|---|---|---|
| Formula | Example | Comment | EP_SP (μM) |
| I | 1 | | 0.11 |
| I | 2 | | 0.064 |
| I | 3 | | 0.022 |
| I | 4 | | 0.015 |
| I | 5 | | 0.039 |
| I | 6 | | 0.32 |
| I | 7 | | 0.008 |
| I | 9 | | 0.059 |
| I | 10 | | 0.031 |
| I | 11 | | 0.13 |
| I | 12 | | 0.029 |
| I | 13 | | 0.24 |
| I | 14 | | 0.078 |
| I | 15 | | 0.11 |
| I | 16 | | 0.24 |
| I | 17 | | 0.1 |
| I | | (S) enantiomer of Example 18 | 0.5 |
| I | 18 | | 0.024 |
| I | 19 | | 0.31 |
| I | 20 | | 0.049 |
| I | 21 | | 0.014 |
| I | 22 | | 0.0091 |
| I | 23 | | 0.11 |
| I | 24 | | 0.1 |
| I | 25 | | 0.015 |
| I | 26 | | 0.19 |
| I | 27 | | 0.09 |
| I | | (S) enantiomer of Example 27 | 0.4 |
| I | 28 | | 0.23 |
| I | 29 | | 0.027 |
| I | 30 | | 0.016 |
| I | 31 | | 0.39 |
| I | 36 | | 0.2 |
| I | 37 | | 0.077 |
| I | 38 | | 0.039 |
| I | 40 | | 0.059 |
| I | 41 | | 0.17 |
| I | 44 | | 0.13 |
| I | 46 | | 0.25 |
| I | 47 | | 0.036 |
| I | 48 | | 0.86 |
| I | 49 | | 0.86 |
| I | 50 | | 0.26 |
| I | 51 | | 0.068 |
| I | 52 | | 0.17 |
| I | 53 | | 0.25 |
| I | 57 | | 0.54 |
| I | 58 | | 0.32 |
| I | 59 | | 0.047 |
| I | 62 | | 0.017 |
| I | 63 | | 0.016 |
| I | 64 | | 0.29 |
| I | 65 | | 0.025 |
| I | 66 | | 0.22 |
| I | 69 | | 0.13 |
| I | 70 | | 0.06 |
| I | 72 | | 0.34 |
| I | 74 | | 0.0055 |
| I | 75 | | 0.12 |
| I | 76 | | 0.032 |
| I | 78 | | 0.1 |
| I | 79 | | 0.1 |
| I | 80 | | 0.7 |
| I | 81 | | 0.14 |
| I | 82 | | 0.03 |
| I | 83 | | 0.092 |
| I | 84 | | 0.69 |
| I | 85 | | 0.016 |
| I | 86 | | 0.1 |
| I | 87 | | 0.39 |
| III | 89 | | 0.34 |
| III | 90 | | 0.4 |
| III | 91 | | 0.79 |
| III | 92 | | 0.069 |
| III | 93 | | 0.11 |
| III | 94 | | 0.24 |
| II | 95 | | 0.17 |
| II | 96 | | 0.0088 |
| II | 97 | | 0.02 |
| II | 99 | | 0.43 |
| II | 101 | | 0.48 |
| II | 102 | | 0.65 |
| II | 103 | | 0.22 |
| | 107 | | 0.99 |
| II | 201 | | 0.37 |
| II | 202 | | 0.022 |
| II | 203 | | 0.074 |
| I | 204 | | 0.01 |
| I | 205 | | 0.039 |
| I | 206 | | 0.05 |
| I | 208 | | 0.88 |
| I | 209 | | 0.11 |
| I | 212 | | 0.087 |
| I | 213 | | 0.42 |
| I | 217 | | 0.37 |
| I | 218 | | 0.56 |
| I | 219 | | 0.65 |
| I | 220 | | 0.014 |
| I | 221 | | 0.016 |
| I | 222 | | 0.033 |
| I | 223 | | 0.039 |
| I | 224 | | 0.04 |
| I | 225 | | 0.01 |
| I | 226 | | 0.75 |
| I | 227 | | 0.02 |
| I | 228 | | 0.21 |
| I | 229 | | 0.012 |
| I | 230 | | 0.063 |
| I | 231 | | 0.43 |
| I | 232 | | 0.88 |
| I | 233 | | 0.87 |
| I | 234 | | 0.34 |
| I | 236 | | 0.039 |
| I | 237 | | 0.05 |
| I | 238 | | 0.084 |
| I | 239 | | 0.025 |
| I | 241 | | 0.065 |
| I | 242 | | 0.11 |
| I | 243 | | 0.67 |
| I | 244 | | 0.77 |
| I | 245 | | 0.69 |
| I | 246 | | 0.23 |
| I | 247 | | 0.052 |
| I | 248 | | 0.76 |
| I | 249 | | 0.11 |
| I | 250 | | 0.0098 |
| I | 251 | | 0.016 |
| I | 252 | | 0.11 |
| I | 253 | | 0.42 |
| I | 254 | | 0.014 |
| I | 255 | | 0.026 |
| I | 256 | | 0.013 |
| II | 257 | | 0.73 |
| II | 258 | | 0.83 |
| I | 259 | | 0.79 |
| I | 260 | | 0.2 |
| I | 261 | | 0.53 |
| I | 262 | | 0.27 |
| I | 263 | | 0.22 |
| I | 264 | | 0.084 |
| I | 265 | | 0.33 |
| I | 266 | | 0.13 |
| I | 267 | | 0.8 |
| I | 268 | | 0.088 |
| I | 269 | | 0.94 |
| I | 270 | | 0.38 |
| I | 271 | | 0.26 |
| I | 272 | | 0.21 |
| I | 273 | | 0.23 |
| I | 274 | | 0.23 |
| I | 275 | | 0.51 |
| I | 276 | | 0.13 |
| I | 277 | | 0.59 |
| I | 278 | | 0.96 |
| I | 279 | | 0.5 |
| I | 280 | | 0.71 |
| I | 281 | | 0.32 |
| I | 282 | | 0.76 |
| I | 283 | | 0.62 |
| I | 284 | | 0.12 |
| I | 285 | | 0.35 |
| I | 286 | | 0.16 |
| I | 287 | | 0.25 |
| I | 288 | | 0.49 |
| I | 289 | | 0.71 |
| I | 290 | | 0.76 |
| I | 291 | | 0.35 |
| I | 292 | | 0.43 |
| I | 293 | | 0.85 |
| I | 294 | | 0.13 |
| I | 295 | | 0.23 |
| I | 296 | | 0.28 |
| I | 297 | | 0.022 |
| I | 298 | | 0.22 |
| I | 299 | | 0.038 |
| I | 300 | | 0.0032 |
| I | 301 | | 0.039 |
| I | 302 | | 0.12 |
| I | 303 | | 0.26 |
| I | 304 | | 0.57 |
| I | 305 | | 0.36 |
| I | 306 | | 0.17 |
| I | 307 | | 0.73 |
| I | 308 | | 0.1 |
| I | 309 | | 0.12 |
| I | 310 | | 0.018 |
| I | 311 | | 0.023 |
| I | 312 | | 0.083 |
| I | 313 | | 0.14 |
| I | 314 | | 0.31 |
| I | 315 | | 0.047 |
| I | 316 | | 0.028 |
| I | 317 | | 0.25 |
| I | 318 | | 0.19 |
| I | 319 | | 0.11 |
| I | 320 | | 0.012 |
| I | 321 | | 0.21 |
| I | 322 | | 0.51 |
| I | 323 | | 0.47 |
| I | 324 | | 0.79 |
| I | 325 | | 0.24 |
| I | 326 | | 0.23 |
| I | 327 | | 0.92 |
| I | 328 | | 0.01 |
| I | 329 | | 1 |
| I | 330 | | 0.62 |
| I | 331 | | 0.14 |
| II | 332 | | 0.22 |
| II | 333 | | 0.065 |

### Example 401: Analgesia Induced by Sodium Channel Blockers

This example describes experimental protocols that can be employed to test efficacy of the compounds disclosed herein.

### Heat Induced Tail Flick Latency Test

In this test, the analgesia effect produced by administering a compound of the invention can be observed through heat-induced tail-flick in mice. The test includes a heat source consisting of a projector lamp with a light beam focused and directed to a point on the tail of a mouse being tested. The tail-flick latencies, which are assessed prior to drug treatment, and in response to a noxious heat stimulus, i.e., the response time from applying radiant heat on the dorsal surface of the tail to the occurrence of tail flick, are measured and recorded at 40, 80, 120, and 160 minutes.

For the first part of this study, 65 animals undergo assessment of baseline tail flick latency once a day over two consecutive days. These animals are then randomly assigned to one of the 11 different treatment groups including a vehicle control, a morphine control, and 9 compounds at 30 mg/Kg are administered intramuscularly. Following dose administration, the animals are closely monitored for signs of toxicity including tremor or seizure, hyperactivity, shallow, rapid or depressed breathing and failure to groom. The optimal incubation time for each compound is determined via regression analysis. The analgesic activity of the test compounds is expressed as a percentage of the maximum possible effect (% MPE) and is calculated using the following formula:

$$\% MPE = \frac{Postdrug latency - Predrug latency}{Cut - off time ( 10 s ) - Predrug latency} \times 100 \%$$

where:
Postdrug latency=the latency time for each individual animal taken before the tail is removed (flicked) from the heat source after receiving drug.
Predrug latency=the latency time for each individual animal taken before the tail is flicked from the heat source prior to receiving drug.
Cut-off time (10 s)=is the maximum exposure to the heat source.

### Acute Pain (Formalin Test)

The formalin test is used as an animal model of acute pain. In the formalin test, animals are briefly habituated to the plexiglass test chamber on the day prior to experimental day for 20 minutes. On the test day, animals are randomly injected with the test articles. At 30 minutes after drug administration, 50 μL of 10% formalin is injected subcutaneously into the plantar surface of the left hind paw of the rats. Video data acquisition begins immediately after formalin administration, for duration of 90 minutes.

The images are captured using the Actimetrix Limelight software which stores files under the *.llii extension, and then converts it into the MPEG-4 coding. The videos are then analyzed using behaviour analysis software “The Observer 5.1”, (Version 5.0, Noldus Information Technology, Wageningen, The Netherlands). The video analysis is conducted by watching the animal behaviour and scoring each according to type, and defining the length of the behaviour (Dubuisson and Dennis, 1977). Scored behaviours include: (1) normal behaviour, (2) putting no weight on the paw, (3) raising the paw, (4) licking/biting or scratching the paw. Elevation, favoring, or excessive licking, biting and scratching of the injected paw indicate a pain response. Analgesic response or protection from compounds is indicated if both paws are resting on the floor with no obvious favoring, excessive licking, biting or scratching of the injected paw.

Analysis of the formalin test data is done according to two factors: (1) Percent Maximal Potential Inhibitory Effect (% MPIE) and (2) pain score. The % MPIEs is calculated by a series of steps, where the first is to sum the length of non-normal behaviours (behaviours 1,2,3) of each animal. A single value for the vehicle group is obtained by averaging all scores within the vehicle treatment group. The following calculation yields the MPIE value for each animal:

MPIE (%)=100−[(treatment sum/average vehicle value)×100%]

The pain score is calculated from a weighted scale as described above. The duration of the behaviour is multiplied by the weight (rating of the severity of the response), and divided by the total length of observation to determine a pain rating for each animal. The calculation is represented by the following formula:

Pain rating=[0(To)+1(T1)+2(T2)+3(T3)]/(To+T1+T2+T3)

### CFA Induced Chronic Inflammatory Pain

In this test, tactile allodynia is assessed with calibrated von Frey filaments. Following a full week of acclimatization to the vivarium facility, 150 μL of the “Complete Freund's Adjuvant” (CFA) emulsion (CFA suspended in an oil/saline (1:1) emulsion at a concentration of 0.5 mg/mL) is injected subcutaneously into the plantar surface of the left hind paw of rats under light isoflurane anaesthesia. Animals are allowed to recover from the anaesthesia and the baseline thermal and mechanical nociceptive thresholds of all animals are assessed one week after the administration of CFA. All animals are habituated to the experimental equipment for 20 minutes on the day prior to the start of the experiment. The test and control articles are administrated to the animals, and the nociceptive thresholds measured at defined time points after drug administration to determine the analgesic responses to each of the six available treatments. The time points used are previously determined to show the highest analgesic effect for each test compound.

Thermal nociceptive thresholds of the animals are assessed using the Hargreaves test. Animals are placed in a Plexiglas enclosure set on top of an elevated glass platform with heating units. The glass platform is thermostatically controlled at a temperature of approximately 30° C. for all test trials. Animals are allowed to accommodate for 20 minutes following placement into the enclosure until all exploration behaviour ceases. The Model 226 Plantar/Tail Stimulator Analgesia Meter (IITC, Woodland Hills, CA) is used to apply a radiant heat beam from underneath the glass platform to the plantar surface of the hind paws. During all test trials, the idle intensity and active intensity of the heat source are set at 1 and 45 respectively, and a cut off time of 20 seconds is employed to prevent tissue damage.

The response thresholds of animals to tactile stimuli are measured using the Model 2290 Electrovonfrey anesthesiometer (IITC Life Science, Woodland Hills, CA) following the Hargreaves test. Animals are placed in an elevated Plexiglas enclosure set on a mire mesh surface. After 10 minutes of accommodation, pre-calibrated Von Frey hairs are applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force is used because it represent approximately 10% of the animals' body weight and it serves to prevent raising of the entire limb due to the use of stiffer hairs, which would change the nature of the stimulus.

### Postoperative Models of Nociception

In this model, the hypealgesia caused by an intra-planar incision in the paw is measured by applying increased tactile stimuli to the paw until the animal withdraws its paw from the applied stimuli. While animals are anaesthetized under 3.5% isofluorane, which is delivered via a nose cone, a 1 cm longitudinal incision is made using a number 10 scalpel blade in the plantar aspect of the left hind paw through the skin and fascia, starting 0.5 cm from the proximal edge of the heel and extending towards the toes. Following the incision, the skin is apposed using 2,3-0 sterilized silk sutures. The injured site is covered with Polysporin and Betadine. Animals are returned to their home cage for overnight recovery.

The withdrawal thresholds of animals to tactile stimuli for both operated (ipsilateral) and unoperated (contralateral) paws can be measured using the Model 2290 Electrovonfrey anesthesiometer (IITC Life Science, Woodland Hills, CA). Animals are placed in an elevated Plexiglas enclosure set on a mire mesh surface. After at least 10 minutes of acclimatization, pre-calibrated Von Frey hairs are applied perpendicularly to the plantar surface of both paws of the animals in an ascending order starting from the 10 g hair, with sufficient force to cause slight buckling of the hair against the paw. Testing continues until the hair with the lowest force to induce a rapid flicking of the paw is determined or when the cut off force of approximately 20 g is reached. This cut off force is used because it represent approximately 10% of the animals' body weight and it serves to prevent raising of the entire limb due to the use of stiffer hairs, which would change the nature of the stimulus.

### Neuropathic Pain Model; Chronic Constriction Injury

Briefly, an approximately 3 cm incision is made through the skin and the fascia at the mid thigh level of the animals' left hind leg using a no. 10 scalpel blade. The left sciatic nerve is exposed via blunt dissection through the biceps femoris with care to minimize haemorrhagia. Four loose ligatures are tied along the sciatic nerve using 4-0 non-degradable sterilized silk sutures at intervals of 1 to 2 mm apart. The tension of the loose ligatures is tight enough to induce slight constriction of the sciatic nerve when viewed under a dissection microscope at a magnification of 4 fold. In the sham-operated animal, the left sciatic nerve is exposed without further manipulation. Antibacterial ointment is applied directly into the wound, and the muscle is closed using sterilized sutures. Betadine is applied onto the muscle and its surroundings, followed by skin closure with surgical clips.

The response thresholds of animals to tactile stimuli are measured using the Model 2290 Electrovonfrey anesthesiometer (IITC Life Science, Woodland Hills, CA). Animals are placed in an elevated Plexiglas enclosure set on a mire mesh surface. After 10 minutes of accommodation, pre-calibrated Von Frey hairs are applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force is used because it represents approximately 10% of the animals' body weight and it serves to prevent raising of the entire limb due to the use of stiffer hairs, which would change the nature of the stimulus.

Thermal nociceptive thresholds of the animals are assessed using the Hargreaves test. Following the measurement of tactile thresholds, animals are placed in a Plexiglass enclosure set on top of an elevated glass platform with heating units. The glass platform is thermostatically controlled at a temperature of approximately 24 to 26° C. for all test trials. Animals are allowed to accommodate for 10 minutes following placement into the enclosure until all exploration behaviour ceases. The Model 226 Plantar/Tail Stimulator Analgesia Meter (IITC, Woodland Hills, CA) is used to apply a radiant heat beam from underneath the glass platform to the plantar surface of the hind paws. During all test trials, the idle intensity and active intensity of the heat source are set at 1 and 55 respectively, and a cut off time of 20 seconds is used to prevent tissue damage.

### Neuropathic Pain Model: Spinal Nerve Ligation

The spinal nerve ligation (SNL) neuropathic pain model is used as an animal (i.e. rat) model of neuropathic pain. In the SNL test, the lumbar roots of spinal nerves L5 and L6 are tightly ligated to cause nerve injury, which results in the development of mechanical hyperalgesia, mechanical allodynia and thermal hypersensitivity. The surgery is performed two weeks before the test day in order for the pain state to fully develop in the animals. Several spinal nerve ligation variations are used to characterize the analgesic properties of a compound of the invention.
Ligation of the L5 spinal nerve;
Ligation of the L5 and L6 spinal nerves;
Ligation and transection of the L5 spinal nerve;
Ligation and transection of the L5 and L6 spinal nerves; or
Mild irritation of the L4 spinal nerve in combination with any one of the above (1)-(4).

While the animals are anaesthetized under 3.5% isofluorane delivered via a nose cone, an approximately 2.5 cm longitudinal incision is made using a number 10 scalpel blade in the skin just lateral to the dorsal midline, using the level of the posterior iliac crests as the midpoint of the incision. Following the incision, the isoflourane is readjusted to maintenance levels (1.5%-2.5%). At mid-sacral region, an incision is made with the scalpel blade, sliding the blade along the side of the vertebral column (in the saggital plane) until the blade hits the sacrum. Scissors tips are introduced through the incision and the muscle and ligaments are removed from the spine to expose 2-3 cm of the vertebral column. The muscle and fascia are cleared from the spinal vertebra in order to locate the point where the nerve exits from the vertebra. A small glass hook is placed medial to the spinal nerves and the spinal nerves are gently elevated from the surrounding tissues. Once the spinal nerves have been isolated, a small length of non-degradable 6-0 sterilized silk thread is wound twice around the ball at the tip of the glass hook and passed back under the nerve. The spinal nerves are then firmly ligated by tying a knot, ensuring that the nerve bulges on both sides of the ligature. The procedure may be repeated as needed. In some animals, the L4 spinal nerve may be lightly rubbed (up to 20 times) with the small glass hook to maximize the development of neuropathic pain. Antibacterial ointment is applied directly into the incision, and the muscle is closed using sterilized sutures. Betadine is applied onto the muscle and its surroundings, followed by skin closure with surgical staples or sterile non-absorbable monofilament 5-0 nylon sutures.

The analgesic effect produced by topical administration of a compound of the invention to the animals can then be observed by measuring the paw withdrawal threshold of animals to mechanical tactile stimuli. These may be measured using either the mechanical allodynia procedure or the mechanical hyperalgesia procedure as described below. After establishment of the appropriate baseline measurements by either method, topical formulation of a compound of the invention is applied on the ipsilateral ankle and foot. The animals are then placed in plastic tunnels for 15 minutes to prevent them from licking the treated area and removing the compound. Animals are placed in the acrylic enclosure for 15 minutes before testing the ipsilateral paw by either of the methods described below, and the responses are recorded at 0.5, 1.0 and 2.0 hour post treatment.

### Mechanical Allodynia Method

The pain threshold of animals to mechanical alloydnia for both operated and control animals can be measured approximately 14 days post-surgery using manual calibrated von Frey filaments as follows. Animals are placed in an elevated plexiglass enclosure set on a mire mesh surface. Animals are allowed to acclimate for 20-30 minutes. Pre-calibrated Von Frey hairs are applied perpendicularly to the plantar surface of the ipsilateral paw of the animals starting from the 2.0 g hair, with sufficient force to cause slight buckling of the hair against the paw to establish the baseline measurements. Stimuli are presented in a consecutive manner, either in an ascending or descending order until the first change in response is noted, after which four additional responses are recorded for a total of six responses. The six responses measured in grams are entered into a formula as described by Chaplan, S. R. et al., J. Neurosci. Methods, 1994 Jul; 53(1):55-63, and a 50% withdrawal threshold is calculated. This constitutes the mechanical allodynia value.

### Mechanical Hyperalgesia Method

The response thresholds of animals to tactile stimuli are measured using the Model 2290 Electrovonfrey anesthesiometer (IITC Life Science, Woodland Hills, CA). Animals are placed in an elevated Plexiglas enclosure set on a wire mesh surface. After 15 minutes of accommodation in this enclosure, a von Frey hair is applied perpendicularly to the plantar surface of the ipsilateral hind paws of the animals, with sufficient force, measured in grams, to elicit a crisp response of the paw. A response indicates a withdrawal from the painful stimulus and constitutes the efficacy endpoint. The data are expressed as percent change from baseline threshold measured in grams.

### Example 402: In Vivo Assay for Treatment of Pruritus

This example describes experimental protocols that can be employed to test efficacy of the compounds disclosed herein.

The compounds of the invention can be evaluated for their activity as antipruritic agents by in vivo test using rodent models. One established model for peripherally elicited pruritus is through the injection of serotonin into the rostral back area (neck) in hairless rats. Prior to serotonin injections (e.g., 2 mg/mL, 50 μL), a dose of a compound of the present invention can be applied systemically through oral, intravenous or intraperitoneal routes or topically to a circular area fixed diameter (e.g. 18 mm). Following dosing, the serotonin injections are given in the area of the topical dosing. After serotonin injection the animal behaviour is monitored by video recording for 20 min-1.5 h, and the number of scratches in this time compared to vehicle treated animals. Thus, application of a compound of the current invention could suppress serotonin-induced scratching in rats.

### Example 403: Cryo-Electron Microscopy Methods

Crystal structures of NaV1.7 receptors, with inhibitor molecules as described herein, were obtained using cryo-electron microscopy methods.

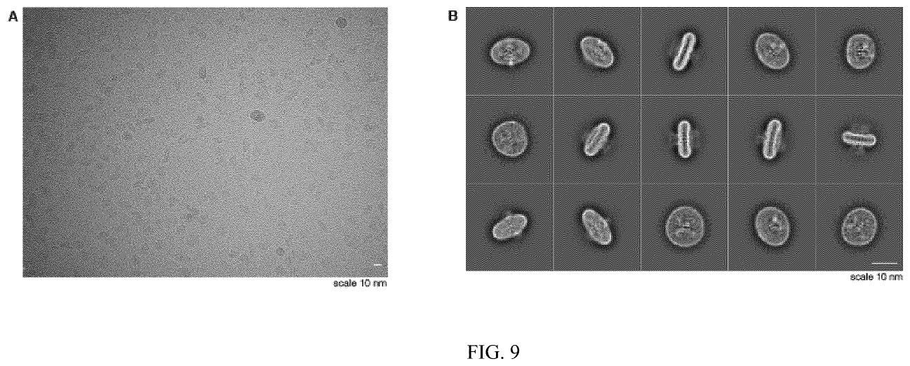

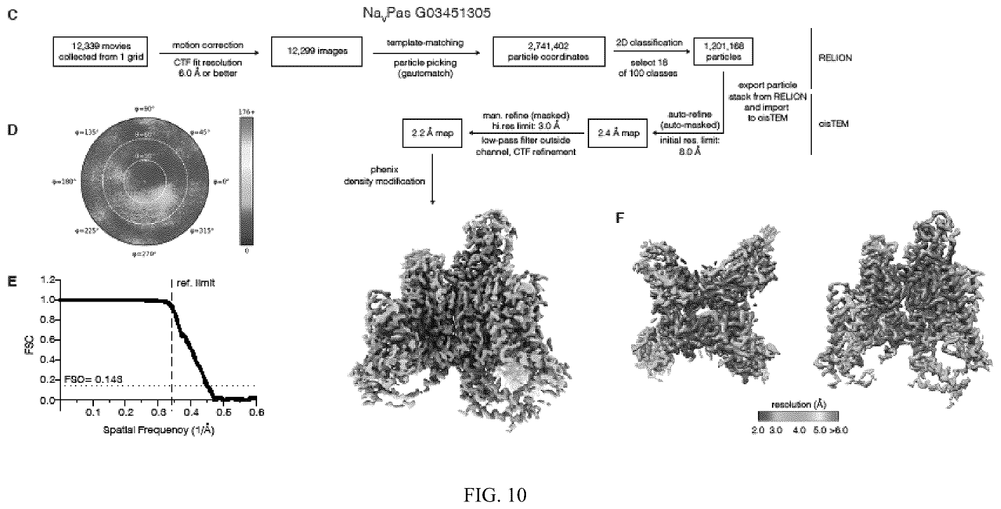

Cryo-EM data were processed using a combination of RELION (Scheres, S. H. RELION: implementation of a Bayesian approach to cryo-EM structure determination. J. Struct. Biol., 180, 519-530, doi:10.1016/j.jsb.2012.09.006 (2012)) and cisTEM (Grant, T., Rohou, A. and Grigorieff, N., cisTEM, user-friendly software for single-particle image processing. Elife 7, doi:10.7554/eLife.35383 (2018)) software packages. Movies were corrected for frame motion using relion's MotionCor2 (Zheng, S. Q. et al. MotionCor2: anisotropic correction of beam-induced motion for improved cryo-electron microscopy. Nat. Methods 14, 331-332, doi: 10.1038/nmeth.4193 (2017)) implementation and contrast-transfer function (CTF) parameters were fit using the 30-4.5 Å band of the spectrum with CTFFIND-4 (Rohou, A. and Grigorieff, N. CTFFIND4: Fast and accurate defocus estimation from electron micrographs. J. Struct. Biol. 192, 216-221, doi:10.1016/j.jsb.2015.08.008 (2015)). For example, for the compound of Example 96, 12,339 movies were motion corrected and filtered based on the detected fit resolution better than 6 Å. Particles were picked using 30 Å low-pass filtered projections of a NavPas 3D reconstruction as template with gautomatch (https://www2.mrc-lmb.cam.ac.uk/research/locally-developed-software/zhang-software/#gauto). Particles were sorted during RELION 2D classification and selected particles were imported into cisTEM for 3D refinements. For the compound of Example 96 2,741,402 particles were isolated from the images resulting from movie alignment and subjected to 2D classification, by dividing them into 100 classes. High-resolution reconstructions were obtained after auto-refine and manual refinement with a mask excluding the nanodisc scaffold proteins and by applying low-pass filter outside the mask (filter resolution 20 Å) and a score threshold of 0.1-0.3. For 3D refinement, which at no point used any data at frequencies higher than 3.0-4.0 Å (i.e., 3.0 Å for NaVPas—Example 96), converged to a high resolution map (2.2 Å in the case of NaVPas—Example 96, using a Fourier shell correlation (FSC)=0.143, determined in cisTEM). For model building and figure preparation, Phenix ResolveCryoEM (Terwilliger T. C., Ludtke S. J., Read R. J., Adams P. D., and Afonine P. V. “Improvement of cryo-EM maps by density modification”, bioRxiv (2019)) density modification was applied. Local resolution was determined in cisTEM using an in-house re-implementation of the blocres algorithm (Cardone, G., Heymann, J. B. and Steven, A. C., One number does not fit all: mapping local variations in resolution in cryo-EM reconstructions. J. Struct. Biol. 184, 226-236, (2013), doi:10.1016/j.jsb.2013.08.002). FIGS. 9 and 10 illustrate aspects of the CryoEM studies of this example.

A PDB-file (Protein Databank format) of atom coordinates for VSD-only residues and small organic molecule bound thereto is found in the Appendix to this specification. The structure illustrates an inhibitor molecule bound in a “hybrid” pose relative to previously known binding configurations for prior inhibitor molecules.

FIGS. 3A, 3B illustrate the “hybrid pose” relative to distinct poses for prior families of NaV1.7 inhibitors, referred to as “aryl” series, and “acyl” series, respectively. It can be seen from these depictions that the binding site comprises two distinct binding regions, approximately at right-angles to one another. One region predominantly accommodates aryl series molecules, whereas the other predominantly fits acyl-series molecules. Based on this understanding, molecules that are “hybrids” or “fusions” or “chimaeras” or molecules having respectively aryl series and acyl series characteristics can be expected to fit simultaneously into both binding regions. Molecules that are able to fit into both binding regions can thereby be expected to have superior binding to molecules that can only fit in one or other of the two binding regions.

All of the U.S. patents, U.S. patent application publications, U.S. patent applications, foreign patents, foreign patent applications and non patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

APPENDIX
PDB File

| REMARK | 3 | | | | |
|---|---|---|---|---|---|
| REMARK | 3 | REFINEMENT. | | | |
| REMARK | 3 | PROGRAM: | PHENIX (1.18.2-3874_final: phenix.real_space_refine) | | |
| REMARK | 3 | AUTHORS: | Adams, Afonine, Bunkoczi, Burnley, Chen, Dar, Davis, | | |
| REMARK | 3 | | : Draizen, Echols, Gildea, Gros, Grosse-Kunstleve, Headd, | | |
| REMARK | 3 | | : Hintze, Hung, Ioerger, Liebschner, McCoy, McKee, Moriarty, | | |
| REMARK | 3 | | : Oeffner, Poon, Read, Richardson, Richardson, Sacchettini, | | |
| REMARK | 3 | | : Sauter, Sobolev, Storoni, Terwilliger, Williams, Zwart | | |
| REMARK | 3 | | | | |
| REMARK | 3 | SCATTERING TABLE: ELECTRON | | | |
| REMARK | 3 | | | | |
| REMARK | 3 | REFINEMENT TARGET: REAL-SPACE (WEIGHTED MAP SUM AT ATOM CENTERS) | | | |
| REMARK | 3 | | | | |
| REMARK | 3 | MODEL TO MAP FIT. | | | |
| REMARK | 3 | CC_mask: | 0.7148 | | |
| REMARK | 3 | CC_volume: | 0.7034 | | |
| REMARK | 3 | CC_peaks: | 0.5910 | | |
| REMARK | 3 | | | | |
| REMARK | 3 | GEOMETRY RESTRAINTS LIBRARY: GEOSTD + MONOMER LIBRARY + CDL V1.2 | | | |
| REMARK | 3 | DEVIATIONS FROM IDEAL VALUES. | | | |
| REMARK | 3 | BOND: | 0.003 | 0.047 | 9826 |
| REMARK | 3 | ANGLE: | 0.651 | 11.559 | 13319 |
| REMARK | 3 | CHIRALITY: | 0.141 | 1.691 | 1538 |
| REMARK | 3 | PLANARITY: | 0.003 | 0.040 | 1573 |
| REMARK | 3 | DIHEDRAL: | 27.882 | 179.999 | 1451 |

| REMARK | 3 | MIN NONBONDED DISTANCE: 1.972 | |
|---|---|---|---|
| REMARK | 3 | | |
| REMARK | 3 | MOLPROBITY STATISTICS. | |
| REMARK | 3 | ALL-ATOM CLASHSCORE: 3.38 | |
| REMARK | 3 | RAMACHANDRAN PLOT: | |
| REMARK | 3 | OUTLIERS: | 0.00% |
| REMARK | 3 | ALLOWED: | 2.08% |
| REMARK | 3 | FAVORED: | 97.92% |

| | | | |
|---|---|---|---|
| REMARK | 3 | ROTAMER OUTLIERS: | 0.30% |
| REMARK | 3 | CBETA DEVIATIONS: | 0.00% |
| REMARK | 3 | PEPTIDE PLANE: | |

| | | | |
|---|---|---|---|
| REMARK | 3 | CIS-PROLINE: | 0.00% |
| REMARK | 3 | CIS-GENERAL: | 0.00% |
| REMARK | 3 | TWISTED PROLINE: | 0.00% |
| REMARK | 3 | TWISTED GENERAL: | 0.00% |
| REMARK | 3 | | |

| | | |
|---|---|---|
| REMARK | 3 | RAMA-Z (RAMACHANDRAN PLOT Z-SCORE): |
| REMARK | 3 | INTERPRETATION: BAD \|RAMA-Z\| > 3; SUSPICIOUS 2 < \|RAMA-Z\| < 3; |

GOOD |RAMA-Z| < 2.

| REMARK | 3 | SCORES FOR WHOLE/HELIX/SHEET/LOOP ARE SCALED INDEPENDENTLY; | |
|---|---|---|---|
| REMARK | 3 | THEREFORE, THE VALUES ARE NOT RELATED IN A SIMPLE MANNER. | |
| REMARK | 3 | WHOLE: | 2.10 (0.26), RESIDUES: 1108 |
| REMARK | 3 | HELIX: | 1.96 (0.18), RESIDUES: 821 |
| REMARK | 3 | SHEET: | −3.37 (0.82), RESIDUES: 22 |
| REMARK | 3 | LOOP: | 0.10 (0.41), RESIDUES: 265 |

| | | | | | |
|---|---|---|---|---|---|
| REMARK | 3 | | | | |
| HELIX | 38 | 38 LYS A 1158 | VAL A 1167 | 1 | 10 |
| HELIX | 39 | 39 GLN A 1170 | MET A 1188 | 1 | 19 |
| HELIX | 40 | 40 GLN A 1196 | THR A 1230 | 1 | 35 |
| HELIX | 41 | 41 GLY A 1232 | GLU A 1255 | 1 | 24 |
| HELIX | 42 | 42 PRO A 1261 | ARG A 1274 | 1 | 14 |
| HELIX | 43 | 43 LEU A 1276 | VAL A 1279 | 5 | 4 |
| HELIX | 44 | 44 LYS A 1283 | LYS A 1294 | 1 | 12 |

| CRYST1 | 112.292 118.158 100.560 90.00 90.00 90.00 P 1 | | | | |
|---|---|---|---|---|---|
| SCALE1 | | 0.008905 | 0.000000 | 0.000000 | 0.00000 |
| SCALE2 | | 0.000000 | 0.008463 | 0.000000 | 0.00000 |
| SCALE3 | | 0.000000 | 0.000000 | 0.009944 | 0.00000 |

| | | | | | | | | | | |
|---|---|---|---|---|---|---|---|---|---|---|
| ATOM | 13796 | N | LYS | A1158 | 109.878 | 186.314 | 162.958 | 1.00 | 29.10 | N |
| ATOM | 13797 | CA | LYS | A1158 | 110.480 | 187.597 | 163.308 | 1.00 | 29.10 | C |
| ATOM | 13798 | C | LYS | A1158 | 111.535 | 187.438 | 164.396 | 1.00 | 29.10 | C |
| ATOM | 13799 | O | LYS | A1158 | 112.648 | 187.961 | 164.277 | 1.00 | 29.10 | O |
| ATOM | 13800 | CB | LYS | A1158 | 109.393 | 188.577 | 163.754 | 1.00 | 29.10 | C |
| ATOM | 13801 | H | LYS | A1158 | 109.032 | 186.265 | 163.101 | 1.00 | 29.10 | H |
| ATOM | 13802 | HA | LYS | A1158 | 110.913 | 187.968 | 162.523 | 1.00 | 29.10 | H |
| ATOM | 13803 | N | ILE | A1159 | 111.206 | 186.712 | 165.466 | 1.00 | 28.02 | N |
| ATOM | 13804 | CA | ILE | A1159 | 112.138 | 186.575 | 166.582 | 1.00 | 28.02 | C |
| ATOM | 13805 | C | ILE | A1159 | 113.352 | 185.754 | 166.163 | 1.00 | 28.02 | C |
| ATOM | 13806 | O | ILE | A1159 | 114.504 | 186.142 | 166.404 | 1.00 | 28.02 | O |
| ATOM | 13807 | CB | ILE | A1159 | 111.427 | 185.951 | 167.798 | 1.00 | 28.02 | C |
| ATOM | 13808 | CG1 | ILE | A1159 | 110.374 | 186.915 | 168.353 | 1.00 | 28.02 | C |
| ATOM | 13809 | CG2 | ILE | A1159 | 112.437 | 185.582 | 168.879 | 1.00 | 28.02 | C |
| ATOM | 13810 | CD1 | ILE | A1159 | 108.988 | 186.717 | 167.771 | 1.00 | 28.02 | C |
| ATOM | 13811 | H | ILE | A1159 | 110.463 | 186.290 | 165.564 | 1.00 | 28.02 | H |
| ATOM | 13812 | HA | ILE | A1159 | 112.451 | 187.456 | 166.839 | 1.00 | 28.02 | H |
| ATOM | 13813 | HB | ILE | A1159 | 110.979 | 185.141 | 167.508 | 1.00 | 28.02 | H |
| ATOM | 13814 | HG12 | ILE | A1159 | 110.308 | 186.794 | 169.313 | 1.00 | 28.02 | H |
| ATOM | 13815 | HG13 | ILE | A1159 | 110.648 | 187.825 | 168.157 | 1.00 | 28.02 | H |
| ATOM | 13816 | HG21 | ILE | A1159 | 111.963 | 185.392 | 169.704 | 1.00 | 28.02 | H |
| ATOM | 13817 | HG22 | ILE | A1159 | 112.932 | 184.796 | 168.600 | 1.00 | 28.02 | H |
| ATOM | 13818 | HG23 | ILE | A1159 | 113.044 | 186.327 | 169.013 | 1.00 | 28.02 | H |
| ATOM | 13819 | HD11 | ILE | A1159 | 108.367 | 187.302 | 168.233 | 1.00 | 28.02 | H |
| ATOM | 13820 | HD12 | ILE | A1159 | 109.003 | 186.935 | 166.826 | 1.00 | 28.02 | H |
| ATOM | 13821 | HD13 | ILE | A1159 | 108.724 | 185.792 | 167.894 | 1.00 | 28.02 | H |
| ATOM | 13822 | N | GLN | A1160 | 113.112 | 184.605 | 165.528 | 1.00 | 25.91 | N |
| ATOM | 13823 | CA | GLN | A1160 | 114.221 | 183.773 | 165.075 | 1.00 | 25.91 | C |
| ATOM | 13824 | C | GLN | A1160 | 115.039 | 184.489 | 164.010 | 1.00 | 25.91 | C |
| ATOM | 13825 | O | GLN | A1160 | 116.267 | 184.375 | 163.985 | 1.00 | 25.91 | O |
| ATOM | 13826 | CB | GLN | A1160 | 113.700 | 182.435 | 164.552 | 1.00 | 25.91 | C |
| ATOM | 13827 | CG | GLN | A1160 | 113.134 | 181.533 | 165.639 | 1.00 | 25.91 | C |
| ATOM | 13828 | CD | GLN | A1160 | 112.556 | 180.244 | 165.088 | 1.00 | 25.91 | C |
| ATOM | 13829 | OE1 | GLN | A1160 | 111.470 | 180.236 | 164.509 | 1.00 | 25.91 | O |
| ATOM | 13830 | NE2 | GLN | A1160 | 113.283 | 179.148 | 165.260 | 1.00 | 25.91 | N |
| ATOM | 13831 | H | GLN | A1160 | 112.331 | 184.292 | 165.351 | 1.00 | 25.91 | H |
| ATOM | 13832 | HA | GLN | A1160 | 114.805 | 183.592 | 165.828 | 1.00 | 25.91 | H |
| ATOM | 13833 | HB2 | GLN | A1160 | 112.994 | 182.603 | 163.909 | 1.00 | 25.91 | H |
| ATOM | 13834 | HB3 | GLN | A1160 | 114.430 | 181.961 | 164.124 | 1.00 | 25.91 | H |
| ATOM | 13835 | HG2 | GLN | A1160 | 113.843 | 181.302 | 166.260 | 1.00 | 25.91 | H |
| ATOM | 13836 | HG3 | GLN | A1160 | 112.426 | 182.004 | 166.104 | 1.00 | 25.91 | H |
| ATOM | 13837 | HE21 | GLN | A1160 | 114.038 | 179.193 | 165.667 | 1.00 | 25.91 | H |
| ATOM | 13838 | HE22 | GLN | A1160 | 112.999 | 178.392 | 164.964 | 1.00 | 25.91 | H |
| ATOM | 13839 | N | GLY | A1161 | 114.378 | 185.232 | 163.121 | 1.00 | 24.52 | N |
| ATOM | 13840 | CA | GLY | A1161 | 115.112 | 185.998 | 162.126 | 1.00 | 24.52 | C |
| ATOM | 13841 | C | GLY | A1161 | 115.994 | 187.066 | 162.746 | 1.00 | 24.52 | C |
| ATOM | 13842 | O | GLY | A1161 | 117.129 | 187.281 | 162.311 | 1.00 | 24.52 | O |
| ATOM | 13843 | H | GLY | A1161 | 113.522 | 185.307 | 163.076 | 1.00 | 24.52 | H |
| ATOM | 13844 | HA2 | GLY | A1161 | 115.673 | 185.400 | 161.608 | 1.00 | 24.52 | H |
| ATOM | 13845 | HA3 | GLY | A1161 | 114.485 | 186.429 | 161.525 | 1.00 | 24.52 | H |
| ATOM | 13846 | N | CYS | A1162 | 115.482 | 187.758 | 163.766 | 1.00 | 27.46 | N |
| ATOM | 13847 | CA | CYS | A1162 | 116.284 | 188.768 | 164.449 | 1.00 | 27.46 | C |
| ATOM | 13848 | C | CYS | A1162 | 117.483 | 188.137 | 165.145 | 1.00 | 27.46 | C |
| ATOM | 13849 | O | CYS | A1162 | 118.598 | 188.672 | 165.088 | 1.00 | 27.46 | O |
| ATOM | 13850 | CB | CYS | A1162 | 115.420 | 189.529 | 165.453 | 1.00 | 27.46 | C |
| ATOM | 13851 | SG | CYS | A1162 | 116.360 | 190.460 | 166.685 | 1.00 | 27.46 | S |
| ATOM | 13852 | H | CYS | A1162 | 114.686 | 187.663 | 164.076 | 1.00 | 27.46 | H |
| ATOM | 13853 | HA | CYS | A1162 | 116.615 | 189.404 | 163.795 | 1.00 | 27.46 | H |
| ATOM | 13854 | HB2 | CYS | A1162 | 114.862 | 190.159 | 164.970 | 1.00 | 27.46 | H |
| ATOM | 13855 | HB3 | CYS | A1162 | 114.860 | 188.894 | 165.927 | 1.00 | 27.46 | H |
| ATOM | 13856 | HG | CYS | A1162 | 115.593 | 191.009 | 167.427 | 1.00 | 27.46 | H |
| ATOM | 13857 | N | ILE | A1163 | 117.274 | 187.000 | 165.812 | 1.00 | 23.24 | N |
| ATOM | 13858 | CA | ILE | A1163 | 118.392 | 186.317 | 166.459 | 1.00 | 23.24 | C |
| ATOM | 13859 | C | ILE | A1163 | 119.401 | 185.855 | 165.412 | 1.00 | 23.24 | C |
| ATOM | 13860 | O | ILE | A1163 | 120.617 | 185.901 | 165.634 | 1.00 | 23.24 | O |
| ATOM | 13861 | CB | ILE | A1163 | 117.877 | 185.149 | 167.322 | 1.00 | 23.24 | C |
| ATOM | 13862 | CG1 | ILE | A1163 | 117.008 | 185.684 | 168.464 | 1.00 | 23.24 | C |
| ATOM | 13863 | CG2 | ILE | A1163 | 119.042 | 184.356 | 167.911 | 1.00 | 23.24 | C |
| ATOM | 13864 | CD1 | ILE | A1163 | 116.450 | 184.610 | 169.383 | 1.00 | 23.24 | C |
| ATOM | 13865 | H | ILE | A1163 | 116.511 | 186.613 | 165.903 | 1.00 | 23.24 | H |
| ATOM | 13866 | HA | ILE | A1163 | 118.843 | 186.944 | 167.047 | 1.00 | 23.24 | H |
| ATOM | 13867 | HB | ILE | A1163 | 117.342 | 184.559 | 166.769 | 1.00 | 23.24 | H |
| ATOM | 13868 | HG12 | ILE | A1163 | 117.544 | 186.282 | 169.008 | 1.00 | 23.24 | H |
| ATOM | 13869 | HG13 | ILE | A1163 | 116.258 | 186.171 | 168.088 | 1.00 | 23.24 | H |
| ATOM | 13870 | HG21 | ILE | A1163 | 118.693 | 183.596 | 168.403 | 1.00 | 23.24 | H |
| ATOM | 13871 | HG22 | ILE | A1163 | 119.617 | 184.040 | 167.201 | 1.00 | 23.24 | H |
| ATOM | 13872 | HG23 | ILE | A1163 | 119.544 | 184.932 | 168.508 | 1.00 | 23.24 | H |
| ATOM | 13873 | HD11 | ILE | A1163 | 115.776 | 185.006 | 169.958 | 1.00 | 23.24 | H |
| ATOM | 13874 | HD12 | ILE | A1163 | 116.052 | 183.909 | 168.842 | 1.00 | 23.24 | H |
| ATOM | 13875 | HD13 | ILE | A1163 | 117.169 | 184.244 | 169.921 | 1.00 | 23.24 | H |
| ATOM | 13876 | N | PHE | A1164 | 118.910 | 185.405 | 164.253 | 1.00 | 23.58 | N |
| ATOM | 13877 | CA | PHE | A1164 | 119.790 | 185.022 | 163.152 | 1.00 | 23.58 | C |
| ATOM | 13878 | C | PHE | A1164 | 120.645 | 186.194 | 162.696 | 1.00 | 23.58 | C |
| ATOM | 13879 | O | PHE | A1164 | 121.857 | 186.052 | 162.498 | 1.00 | 23.58 | O |
| ATOM | 13880 | CB | PHE | A1164 | 118.946 | 184.496 | 161.990 | 1.00 | 23.58 | C |
| ATOM | 13881 | CG | PHE | A1164 | 119.698 | 183.616 | 161.034 | 1.00 | 23.58 | C |
| ATOM | 13882 | CD1 | PHE | A1164 | 119.744 | 182.245 | 161.222 | 1.00 | 23.58 | C |
| ATOM | 13883 | CD2 | PHE | A1164 | 120.340 | 184.161 | 159.932 | 1.00 | 23.58 | C |
| ATOM | 13884 | CE1 | PHE | A1164 | 120.429 | 181.433 | 160.339 | 1.00 | 23.58 | C |
| ATOM | 13885 | CE2 | PHE | A1164 | 121.025 | 183.354 | 159.044 | 1.00 | 23.58 | C |
| ATOM | 13886 | CZ | PHE | A1164 | 121.069 | 181.989 | 159.248 | 1.00 | 23.58 | C |
| ATOM | 13887 | H | PHE | A1164 | 118.072 | 185.312 | 164.084 | 1.00 | 23.58 | H |
| ATOM | 13888 | HA | PHE | A1164 | 120.383 | 184.312 | 163.442 | 1.00 | 23.58 | H |
| ATOM | 13889 | HB2 | PHE | A1164 | 118.213 | 183.974 | 162.352 | 1.00 | 23.58 | H |
| ATOM | 13890 | HB3 | PHE | A1164 | 118.597 | 185.249 | 161.488 | 1.00 | 23.58 | H |
| ATOM | 13891 | HD1 | PHE | A1164 | 119.315 | 181.866 | 161.955 | 1.00 | 23.58 | H |
| ATOM | 13892 | HD2 | PHE | A1164 | 120.312 | 185.081 | 159.793 | 1.00 | 23.58 | H |
| ATOM | 13893 | HE1 | PHE | A1164 | 120.457 | 180.512 | 160.478 | 1.00 | 23.58 | H |
| ATOM | 13894 | HE2 | PHE | A1164 | 121.457 | 183.728 | 158.309 | 1.00 | 23.58 | H |
| ATOM | 13895 | HZ | PHE | A1164 | 121.530 | 181.446 | 158.651 | 1.00 | 23.58 | H |
| ATOM | 13896 | N | ASP | A1165 | 120.029 | 187.363 | 162.523 | 1.00 | 24.66 | N |
| ATOM | 13897 | CA | ASP | A1165 | 120.787 | 188.540 | 162.114 | 1.00 | 24.66 | C |
| ATOM | 13898 | C | ASP | A1165 | 121.819 | 188.918 | 163.169 | 1.00 | 24.66 | C |
| ATOM | 13899 | O | ASP | A1165 | 122.952 | 189.286 | 162.838 | 1.00 | 24.66 | O |
| ATOM | 13900 | CB | ASP | A1165 | 119.838 | 189.708 | 161.846 | 1.00 | 24.66 | C |
| ATOM | 13901 | CG | ASP | A1165 | 118.948 | 189.471 | 160.642 | 1.00 | 24.66 | C |
| ATOM | 13902 | OD1 | ASP | A1165 | 119.373 | 188.744 | 159.720 | 1.00 | 24.66 | O |
| ATOM | 13903 | OD2 | ASP | A1165 | 117.822 | 190.012 | 160.618 | 1.00 | 24.66 | O |
| ATOM | 13904 | H | ASP | A1165 | 119.186 | 187.497 | 162.634 | 1.00 | 24.66 | H |
| ATOM | 13905 | HA | ASP | A1165 | 121.260 | 188.342 | 161.290 | 1.00 | 24.66 | H |
| ATOM | 13906 | HB2 | ASP | A1165 | 119.269 | 189.839 | 162.621 | 1.00 | 24.66 | H |
| ATOM | 13907 | HB3 | ASP | A1165 | 120.361 | 190.508 | 161.679 | 1.00 | 24.66 | H |
| ATOM | 13908 | N | LEU | A1166 | 121.444 | 188.829 | 164.447 | 1.00 | 23.50 | N |
| ATOM | 13909 | CA | LEU | A1166 | 122.362 | 189.211 | 165.516 | 1.00 | 23.50 | C |
| ATOM | 13910 | C | LEU | A1166 | 123.525 | 188.234 | 165.649 | 1.00 | 23.50 | C |
| ATOM | 13911 | O | LEU | A1166 | 124.631 | 188.640 | 166.020 | 1.00 | 23.50 | O |
| ATOM | 13912 | CB | LEU | A1166 | 121.608 | 189.308 | 166.842 | 1.00 | 23.50 | C |
| ATOM | 13913 | CG | LEU | A1166 | 121.110 | 190.701 | 167.230 | 1.00 | 23.50 | C |
| ATOM | 13914 | CD1 | LEU | A1166 | 119.873 | 190.600 | 168.109 | 1.00 | 23.50 | C |
| ATOM | 13915 | CD2 | LEU | A1166 | 122.206 | 191.488 | 167.931 | 1.00 | 23.50 | C |
| ATOM | 13916 | H | LEU | A1166 | 120.675 | 188.554 | 164.715 | 1.00 | 23.50 | H |
| ATOM | 13917 | HA | LEU | A1166 | 122.729 | 190.086 | 165.316 | 1.00 | 23.50 | H |
| ATOM | 13918 | HB2 | LEU | A1166 | 120.834 | 188.725 | 166.798 | 1.00 | 23.50 | H |
| ATOM | 13919 | HB3 | LEU | A1166 | 122.198 | 189.004 | 167.550 | 1.00 | 23.50 | H |
| ATOM | 13920 | HG | LEU | A1166 | 120.865 | 191.183 | 166.425 | 1.00 | 23.50 | H |
| ATOM | 13921 | HD11 | LEU | A1166 | 119.575 | 191.495 | 168.338 | 1.00 | 23.50 | H |
| ATOM | 13922 | HD12 | LEU | A1166 | 119.177 | 190.134 | 167.622 | 1.00 | 23.50 | H |
| ATOM | 13923 | HD13 | LEU | A1166 | 120.099 | 190.110 | 168.915 | 1.00 | 23.50 | H |
| ATOM | 13924 | HD21 | LEU | A1166 | 121.841 | 192.332 | 168.239 | 1.00 | 23.50 | H |
| ATOM | 13925 | HD22 | LEU | A1166 | 122.531 | 190.973 | 168.687 | 1.00 | 23.50 | H |
| ATOM | 13926 | HD23 | LEU | A1166 | 122.929 | 191.649 | 167.305 | 1.00 | 23.50 | H |
| ATOM | 13927 | N | VAL | A1167 | 123.301 | 186.952 | 165.354 | 1.00 | 20.50 | N |
| ATOM | 13928 | CA | VAL | A1167 | 124.310 | 185.931 | 165.619 | 1.00 | 20.50 | C |
| ATOM | 13929 | C | VAL | A1167 | 125.281 | 185.728 | 164.459 | 1.00 | 20.50 | C |
| ATOM | 13930 | O | VAL | A1167 | 126.412 | 185.273 | 164.684 | 1.00 | 20.50 | O |
| ATOM | 13931 | CB | VAL | A1167 | 123.621 | 184.600 | 165.977 | 1.00 | 20.50 | C |
| ATOM | 13932 | CG1 | VAL | A1167 | 124.530 | 183.404 | 165.691 | 1.00 | 20.50 | C |
| ATOM | 13933 | CG2 | VAL | A1167 | 123.186 | 184.607 | 167.435 | 1.00 | 20.50 | C |
| ATOM | 13934 | H | VAL | A1167 | 122.576 | 186.651 | 165.002 | 1.00 | 20.50 | H |
| ATOM | 13935 | HA | VAL | A1167 | 124.831 | 186.206 | 166.390 | 1.00 | 20.50 | H |
| ATOM | 13936 | HB | VAL | A1167 | 122.825 | 184.504 | 165.431 | 1.00 | 20.50 | H |
| ATOM | 13937 | HG11 | VAL | A1167 | 124.153 | 182.621 | 166.124 | 1.00 | 20.50 | H |
| ATOM | 13938 | HG12 | VAL | A1167 | 124.578 | 183.252 | 164.734 | 1.00 | 20.50 | H |
| ATOM | 13939 | HG13 | VAL | A1167 | 125.413 | 183.578 | 166.051 | 1.00 | 20.50 | H |
| ATOM | 13940 | HG21 | VAL | A1167 | 122.777 | 183.754 | 167.648 | 1.00 | 20.50 | H |
| ATOM | 13941 | HG22 | VAL | A1167 | 123.965 | 184.749 | 167.996 | 1.00 | 20.50 | H |
| ATOM | 13942 | HG23 | VAL | A1167 | 122.547 | 185.324 | 167.570 | 1.00 | 20.50 | H |
| ATOM | 13943 | N | THR | A1168 | 124.885 | 186.065 | 163.230 | 1.00 | 22.64 | N |
| ATOM | 13944 | CA | THR | A1168 | 125.692 | 185.793 | 162.046 | 1.00 | 22.64 | C |
| ATOM | 13945 | C | THR | A1168 | 126.568 | 186.975 | 161.640 | 1.00 | 22.64 | C |
| ATOM | 13946 | O | THR | A1168 | 126.834 | 187.160 | 160.445 | 1.00 | 22.64 | O |
| ATOM | 13947 | CB | THR | A1168 | 124.790 | 185.383 | 160.881 | 1.00 | 22.64 | C |
| ATOM | 13948 | OG1 | THR | A1168 | 123.842 | 186.425 | 160.615 | 1.00 | 22.64 | O |
| ATOM | 13949 | CG2 | THR | A1168 | 124.049 | 184.093 | 161.204 | 1.00 | 22.64 | C |
| ATOM | 13950 | H | THR | A1168 | 124.140 | 186.458 | 163.057 | 1.00 | 22.64 | H |
| ATOM | 13951 | HA | THR | A1168 | 126.281 | 185.046 | 162.237 | 1.00 | 22.64 | H |
| ATOM | 13952 | HB | THR | A1168 | 125.332 | 185.233 | 160.091 | 1.00 | 22.64 | H |
| ATOM | 13953 | HG1 | THR | A1168 | 123.336 | 186.530 | 161.277 | 1.00 | 22.64 | H |
| ATOM | 13954 | HG21 | THR | A1168 | 123.508 | 183.823 | 160.445 | 1.00 | 22.64 | H |
| ATOM | 13955 | HG22 | THR | A1168 | 124.684 | 183.388 | 161.403 | 1.00 | 22.64 | H |
| ATOM | 13956 | HG23 | THR | A1168 | 123.472 | 184.224 | 161.972 | 1.00 | 22.64 | H |
| ATOM | 13957 | N | ASN | A1169 | 127.023 | 187.778 | 162.597 | 1.00 | 25.59 | N |
| ATOM | 13958 | CA | ASN | A1169 | 127.870 | 188.929 | 162.324 | 1.00 | 25.59 | C |
| ATOM | 13959 | C | ASN | A1169 | 129.243 | 188.741 | 162.959 | 1.00 | 25.59 | C |
| ATOM | 13960 | O | ASN | A1169 | 129.413 | 187.986 | 163.919 | 1.00 | 25.59 | O |
| ATOM | 13961 | CB | ASN | A1169 | 127.223 | 190.218 | 162.844 | 1.00 | 25.59 | C |
| ATOM | 13962 | CG | ASN | A1169 | 127.877 | 191.465 | 162.283 | 1.00 | 25.59 | C |
| ATOM | 13963 | OD1 | ASN | A1169 | 128.396 | 191.460 | 161.167 | 1.00 | 25.59 | O |
| ATOM | 13964 | ND2 | ASN | A1169 | 127.850 | 192.545 | 163.055 | 1.00 | 25.59 | N |
| ATOM | 13965 | H | ASN | A1169 | 126.850 | 187.671 | 163.433 | 1.00 | 25.59 | H |
| ATOM | 13966 | HA | ASN | A1169 | 127.992 | 189.019 | 161.366 | 1.00 | 25.59 | H |
| ATOM | 13967 | HB2 | ASN | A1169 | 126.288 | 190.228 | 162.588 | 1.00 | 25.59 | H |
| ATOM | 13968 | HB3 | ASN | A1169 | 127.304 | 190.245 | 163.810 | 1.00 | 25.59 | H |
| ATOM | 13969 | HD21 | ASN | A1169 | 127.477 | 192.512 | 163.829 | 1.00 | 25.59 | H |
| ATOM | 13970 | HD22 | ASN | A1169 | 128.207 | 193.278 | 162.781 | 1.00 | 25.59 | H |
| ATOM | 13971 | N | GLN | A1170 | 130.231 | 189.448 | 162.400 | 1.00 | 25.54 | N |
| ATOM | 13972 | CA | GLN | A1170 | 131.598 | 189.341 | 162.901 | 1.00 | 25.54 | C |
| ATOM | 13973 | C | GLN | A1170 | 131.706 | 189.808 | 164.347 | 1.00 | 25.54 | C |
| ATOM | 13974 | O | GLN | A1170 | 132.553 | 189.309 | 165.100 | 1.00 | 25.54 | O |
| ATOM | 13975 | CB | GLN | A1170 | 132.544 | 190.157 | 162.018 | 1.00 | 25.54 | C |
| ATOM | 13976 | CG | GLN | A1170 | 132.659 | 189.656 | 160.587 | 1.00 | 25.54 | C |
| ATOM | 13977 | CD | GLN | A1170 | 133.224 | 188.253 | 160.500 | 1.00 | 25.54 | C |
| ATOM | 13978 | OE1 | GLN | A1170 | 134.136 | 187.891 | 161.244 | 1.00 | 25.54 | O |
| ATOM | 13979 | NE2 | GLN | A1170 | 132.688 | 187.454 | 159.585 | 1.00 | 25.54 | N |
| ATOM | 13980 | H | GLN | A1170 | 130.134 | 189.989 | 161.739 | 1.00 | 25.54 | H |
| ATOM | 13981 | HA | GLN | A1170 | 131.876 | 188.412 | 162.866 | 1.00 | 25.54 | H |
| ATOM | 13982 | HB2 | GLN | A1170 | 132.224 | 191.072 | 161.985 | 1.00 | 25.54 | H |
| ATOM | 13983 | HB3 | GLN | A1170 | 133.431 | 190.134 | 162.410 | 1.00 | 25.54 | H |
| ATOM | 13984 | HG2 | GLN | A1170 | 131.777 | 189.650 | 160.182 | 1.00 | 25.54 | H |
| ATOM | 13985 | HG3 | GLN | A1170 | 133.247 | 190.248 | 160.092 | 1.00 | 25.54 | H |
| ATOM | 13986 | HE21 | GLN | A1170 | 132.052 | 187.743 | 159.082 | 1.00 | 25.54 | H |
| ATOM | 13987 | HE22 | GLN | A1170 | 132.974 | 186.649 | 159.495 | 1.00 | 25.54 | H |
| ATOM | 13988 | N | ALA | A1171 | 130.866 | 190.764 | 164.749 | 1.00 | 21.39 | N |
| ATOM | 13989 | CA | ALA | A1171 | 130.940 | 191.289 | 166.108 | 1.00 | 21.39 | C |
| ATOM | 13990 | C | ALA | A1171 | 130.718 | 190.189 | 167.136 | 1.00 | 21.39 | C |
| ATOM | 13991 | O | ALA | A1171 | 131.395 | 190.149 | 168.169 | 1.00 | 21.39 | O |
| ATOM | 13992 | CB | ALA | A1171 | 129.917 | 192.409 | 166.292 | 1.00 | 21.39 | C |
| ATOM | 13993 | H | ALA | A1171 | 130.254 | 191.119 | 164.261 | 1.00 | 21.39 | H |
| ATOM | 13994 | HA | ALA | A1171 | 131.824 | 191.663 | 166.254 | 1.00 | 21.39 | H |
| ATOM | 13995 | HB1 | ALA | A1171 | 129.979 | 192.747 | 167.199 | 1.00 | 21.39 | H |
| ATOM | 13996 | HB2 | ALA | A1171 | 130.110 | 193.120 | 165.660 | 1.00 | 21.39 | H |
| ATOM | 13997 | HB3 | ALA | A1171 | 129.029 | 192.055 | 166.130 | 1.00 | 21.39 | H |
| ATOM | 13998 | N | PHE | A1172 | 129.771 | 189.287 | 166.872 | 1.00 | 17.35 | N |
| ATOM | 13999 | CA | PHE | A1172 | 129.500 | 188.197 | 167.804 | 1.00 | 17.35 | C |
| ATOM | 14000 | C | PHE | A1172 | 130.732 | 187.318 | 167.994 | 1.00 | 17.35 | C |
| ATOM | 14001 | O | PHE | A1172 | 131.119 | 187.001 | 169.127 | 1.00 | 17.35 | O |
| ATOM | 14002 | CB | PHE | A1172 | 128.318 | 187.373 | 167.292 | 1.00 | 17.35 | C |
| ATOM | 14003 | CG | PHE | A1172 | 127.697 | 186.486 | 168.331 | 1.00 | 17.35 | C |
| ATOM | 14004 | CD1 | PHE | A1172 | 126.917 | 187.021 | 169.341 | 1.00 | 17.35 | C |
| ATOM | 14005 | CD2 | PHE | A1172 | 127.882 | 185.115 | 168.288 | 1.00 | 17.35 | C |
| ATOM | 14006 | CE1 | PHE | A1172 | 126.342 | 186.205 | 170.296 | 1.00 | 17.35 | C |
| ATOM | 14007 | CE2 | PHE | A1172 | 127.309 | 184.295 | 169.238 | 1.00 | 17.35 | C |
| ATOM | 14008 | CZ | PHE | A1172 | 126.538 | 184.841 | 170.244 | 1.00 | 17.35 | C |
| ATOM | 14009 | H | PHE | A1172 | 129.278 | 189.285 | 166.167 | 1.00 | 17.35 | H |
| ATOM | 14010 | HA | PHE | A1172 | 129.258 | 188.569 | 168.666 | 1.00 | 17.35 | H |
| ATOM | 14011 | HB2 | PHE | A1172 | 127.631 | 187.978 | 166.971 | 1.00 | 17.35 | H |
| ATOM | 14012 | HB3 | PHE | A1172 | 128.623 | 186.808 | 166.565 | 1.00 | 17.35 | H |
| ATOM | 14013 | HD1 | PHE | A1172 | 126.783 | 187.941 | 169.381 | 1.00 | 17.35 | H |
| ATOM | 14014 | HD2 | PHE | A1172 | 128.402 | 184.742 | 167.613 | 1.00 | 17.35 | H |
| ATOM | 14015 | HE1 | PHE | A1172 | 125.822 | 186.575 | 170.972 | 1.00 | 17.35 | H |
| ATOM | 14016 | HE2 | PHE | A1172 | 127.443 | 183.375 | 169.201 | 1.00 | 17.35 | H |
| ATOM | 14017 | HZ | PHE | A1172 | 126.151 | 184.289 | 170.885 | 1.00 | 17.35 | H |
| ATOM | 14018 | N | ASP | A1173 | 131.370 | 186.920 | 166.891 | 1.00 | 18.37 | N |
| ATOM | 14019 | CA | ASP | A1173 | 132.546 | 186.062 | 166.986 | 1.00 | 18.37 | C |
| ATOM | 14020 | C | ASP | A1173 | 133.692 | 186.773 | 167.695 | 1.00 | 18.37 | C |
| ATOM | 14021 | O | ASP | A1173 | 134.392 | 186.173 | 168.521 | 1.00 | 18.37 | O |
| ATOM | 14022 | CB | ASP | A1173 | 132.979 | 185.610 | 165.591 | 1.00 | 18.37 | C |
| ATOM | 14023 | CG | ASP | A1173 | 131.837 | 185.018 | 164.789 | 1.00 | 18.37 | C |
| ATOM | 14024 | OD1 | ASP | A1173 | 130.666 | 185.284 | 165.133 | 1.00 | 18.37 | O |
| ATOM | 14025 | OD2 | ASP | A1173 | 132.110 | 184.287 | 163.814 | 1.00 | 18.37 | O |
| ATOM | 14026 | H | ASP | A1173 | 131.143 | 187.132 | 166.088 | 1.00 | 18.37 | H |
| ATOM | 14027 | HA | ASP | A1173 | 132.320 | 185.271 | 167.501 | 1.00 | 18.37 | H |
| ATOM | 14028 | HB2 | ASP | A1173 | 133.323 | 186.375 | 165.104 | 1.00 | 18.37 | H |
| ATOM | 14029 | HB3 | ASP | A1173 | 133.668 | 184.933 | 165.677 | 1.00 | 18.37 | H |
| ATOM | 14030 | N | ILE | A1174 | 133.905 | 188.053 | 167.380 | 1.00 | 15.56 | N |
| ATOM | 14031 | CA | ILE | A1174 | 134.981 | 188.798 | 168.029 | 1.00 | 15.56 | C |
| ATOM | 14032 | C | ILE | A1174 | 134.714 | 188.913 | 169.525 | 1.00 | 15.56 | C |
| ATOM | 14033 | O | ILE | A1174 | 135.635 | 188.815 | 170.345 | 1.00 | 15.56 | O |
| ATOM | 14034 | CB | ILE | A1174 | 135.152 | 190.177 | 167.364 | 1.00 | 15.56 | C |
| ATOM | 14035 | CG1 | ILE | A1174 | 135.657 | 189.992 | 165.926 | 1.00 | 15.56 | C |
| ATOM | 14036 | CG2 | ILE | A1174 | 136.101 | 191.064 | 168.184 | 1.00 | 15.56 | C |
| ATOM | 14037 | CD1 | ILE | A1174 | 136.448 | 191.159 | 165.361 | 1.00 | 15.56 | C |
| ATOM | 14038 | H | ILE | A1174 | 133.451 | 188.504 | 166.806 | 1.00 | 15.56 | H |
| ATOM | 14039 | HA | ILE | A1174 | 135.811 | 188.309 | 167.916 | 1.00 | 15.56 | H |
| ATOM | 14040 | HB | ILE | A1174 | 134.284 | 190.609 | 167.330 | 1.00 | 15.56 | H |
| ATOM | 14041 | HG12 | ILE | A1174 | 136.231 | 189.211 | 165.897 | 1.00 | 15.56 | H |
| ATOM | 14042 | HG13 | ILE | A1174 | 134.892 | 189.852 | 165.346 | 1.00 | 15.56 | H |
| ATOM | 14043 | HG21 | ILE | A1174 | 136.160 | 191.941 | 167.774 | 1.00 | 15.56 | H |
| ATOM | 14044 | HG22 | ILE | A1174 | 135.760 | 191.166 | 169.086 | 1.00 | 15.56 | H |
| ATOM | 14045 | HG23 | ILE | A1174 | 136.978 | 190.650 | 168.207 | 1.00 | 15.56 | H |
| ATOM | 14046 | HD11 | ILE | A1174 | 136.568 | 191.024 | 164.408 | 1.00 | 15.56 | H |
| ATOM | 14047 | HD12 | ILE | A1174 | 135.956 | 191.980 | 165.518 | 1.00 | 15.56 | H |
| ATOM | 14048 | HD13 | ILE | A1174 | 137.312 | 191.200 | 165.799 | 1.00 | 15.56 | H |
| ATOM | 14049 | N | SER | A1175 | 133.452 | 189.126 | 169.905 | 1.00 | 13.45 | N |
| ATOM | 14050 | CA | SER | A1175 | 133.108 | 189.200 | 171.321 | 1.00 | 13.45 | C |
| ATOM | 14051 | C | SER | A1175 | 133.358 | 187.869 | 172.017 | 1.00 | 13.45 | C |
| ATOM | 14052 | O | SER | A1175 | 133.837 | 187.836 | 173.157 | 1.00 | 13.45 | O |
| ATOM | 14053 | CB | SER | A1175 | 131.649 | 189.625 | 171.480 | 1.00 | 13.45 | C |
| ATOM | 14054 | OG | SER | A1175 | 131.338 | 190.703 | 170.616 | 1.00 | 13.45 | O |
| ATOM | 14055 | H | SER | A1175 | 132.787 | 189.228 | 169.369 | 1.00 | 13.45 | H |
| ATOM | 14056 | HA | SER | A1175 | 133.665 | 189.871 | 171.746 | 1.00 | 13.45 | H |
| ATOM | 14057 | HB2 | SER | A1175 | 131.076 | 188.872 | 171.265 | 1.00 | 13.45 | H |
| ATOM | 14058 | HB3 | SER | A1175 | 131.500 | 189.902 | 172.398 | 1.00 | 13.45 | H |
| ATOM | 14059 | HG | SER | A1175 | 131.837 | 191.358 | 170.783 | 1.00 | 13.45 | H |
| ATOM | 14060 | N | ILE | A1176 | 133.029 | 186.759 | 171.354 | 1.00 | 9.36 | N |
| ATOM | 14061 | CA | ILE | A1176 | 133.299 | 185.447 | 171.941 | 1.00 | 9.36 | C |
| ATOM | 14062 | C | ILE | A1176 | 134.798 | 185.251 | 172.137 | 1.00 | 9.36 | C |
| ATOM | 14063 | O | ILE | A1176 | 135.245 | 184.735 | 173.170 | 1.00 | 9.36 | O |
| ATOM | 14064 | CB | ILE | A1176 | 132.689 | 184.331 | 171.071 | 1.00 | 9.36 | C |
| ATOM | 14065 | CG1 | ILE | A1176 | 131.160 | 184.441 | 171.039 | 1.00 | 9.36 | C |
| ATOM | 14066 | CG2 | ILE | A1176 | 133.116 | 182.962 | 171.582 | 1.00 | 9.36 | C |
| ATOM | 14067 | CD1 | ILE | A1176 | 130.490 | 184.330 | 172.398 | 1.00 | 9.36 | C |
| ATOM | 14068 | H | ILE | A1176 | 132.656 | 186.737 | 170.579 | 1.00 | 9.36 | H |
| ATOM | 14069 | HA | ILE | A1176 | 132.887 | 185.407 | 172.818 | 1.00 | 9.36 | H |
| ATOM | 14070 | HB | ILE | A1176 | 133.021 | 184.436 | 170.165 | 1.00 | 9.36 | H |
| ATOM | 14071 | HG12 | ILE | A1176 | 130.917 | 185.301 | 170.668 | 1.00 | 9.36 | H |
| ATOM | 14072 | HG13 | ILE | A1176 | 130.809 | 183.732 | 170.478 | 1.00 | 9.36 | H |
| ATOM | 14073 | HG21 | ILE | A1176 | 132.527 | 182.289 | 171.206 | 1.00 | 9.36 | H |
| ATOM | 14074 | HG22 | ILE | A1176 | 134.030 | 182.791 | 171.307 | 1.00 | 9.36 | H |
| ATOM | 14075 | HG23 | ILE | A1176 | 133.052 | 182.953 | 172.550 | 1.00 | 9.36 | H |
| ATOM | 14076 | HD11 | ILE | A1176 | 129.531 | 184.271 | 172.270 | 1.00 | 9.36 | H |
| ATOM | 14077 | HD12 | ILE | A1176 | 130.811 | 183.533 | 172.849 | 1.00 | 9.36 | H |
| ATOM | 14078 | HD13 | ILE | A1176 | 130.703 | 185.116 | 172.924 | 1.00 | 9.36 | H |
| ATOM | 14079 | N | MET | A1177 | 135.599 | 185.652 | 171.146 | 1.00 | 12.43 | N |
| ATOM | 14080 | CA | MET | A1177 | 137.050 | 185.549 | 171.286 | 1.00 | 12.43 | C |
| ATOM | 14081 | C | MET | A1177 | 137.552 | 186.395 | 172.452 | 1.00 | 12.43 | C |
| ATOM | 14082 | O | MET | A1177 | 138.422 | 185.962 | 173.220 | 1.00 | 12.43 | O |
| ATOM | 14083 | CB | MET | A1177 | 137.735 | 185.976 | 169.986 | 1.00 | 12.43 | C |
| ATOM | 14084 | CG | MET | A1177 | 137.323 | 185.171 | 168.760 | 1.00 | 12.43 | C |
| ATOM | 14085 | SD | MET | A1177 | 138.314 | 183.682 | 168.529 | 1.00 | 12.43 | S |
| ATOM | 14086 | CE | MET | A1177 | 137.257 | 182.449 | 169.281 | 1.00 | 12.43 | C |
| ATOM | 14087 | H | MET | A1177 | 135.332 | 185.981 | 170.398 | 1.00 | 12.43 | H |
| ATOM | 14088 | HA | MET | A1177 | 137.282 | 184.622 | 171.452 | 1.00 | 12.43 | H |
| ATOM | 14089 | HB2 | MET | A1177 | 137.520 | 186.906 | 169.814 | 1.00 | 12.43 | H |
| ATOM | 14090 | HB3 | MET | A1177 | 138.693 | 185.876 | 170.095 | 1.00 | 12.43 | H |
| ATOM | 14091 | HG2 | MET | A1177 | 136.398 | 184.898 | 168.854 | 1.00 | 12.43 | H |
| ATOM | 14092 | HG3 | MET | A1177 | 137.427 | 185.725 | 167.971 | 1.00 | 12.43 | H |
| ATOM | 14093 | HE1 | MET | A1177 | 137.683 | 181.580 | 169.205 | 1.00 | 12.43 | H |
| ATOM | 14094 | HE2 | MET | A1177 | 137.127 | 182.673 | 170.215 | 1.00 | 12.43 | H |
| ATOM | 14095 | HE3 | MET | A1177 | 136.404 | 182.441 | 168.820 | 1.00 | 12.43 | H |
| ATOM | 14096 | N | VAL | A1178 | 137.018 | 187.610 | 172.595 | 1.00 | 7.81 | N |
| ATOM | 14097 | CA | VAL | A1178 | 137.426 | 188.480 | 173.695 | 1.00 | 7.81 | C |
| ATOM | 14098 | C | VAL | A1178 | 137.065 | 187.846 | 175.033 | 1.00 | 1.81 | C |
| ATOM | 14099 | O | VAL | A1178 | 137.838 | 187.907 | 175.997 | 1.00 | 7.81 | O |
| ATOM | 14100 | CB | VAL | A1178 | 136.786 | 189.872 | 173.535 | 1.00 | 7.81 | C |
| ATOM | 14101 | CG1 | VAL | A1178 | 136.852 | 190.648 | 174.844 | 1.00 | 7.81 | C |
| ATOM | 14102 | CG2 | VAL | A1178 | 137.465 | 190.642 | 172.412 | 1.00 | 7.81 | C |
| ATOM | 14103 | H | VAL | A1178 | 136.424 | 187.949 | 172.073 | 1.00 | 7.81 | H |
| ATOM | 14104 | HA | VAL | A1178 | 138.389 | 188.590 | 173.668 | 1.00 | 7.81 | H |
| ATOM | 14105 | HB | VAL | A1178 | 135.851 | 189.763 | 173.300 | 1.00 | 7.81 | H |
| ATOM | 14106 | HG11 | VAL | A1178 | 136.714 | 191.590 | 174.656 | 1.00 | 7.81 | H |
| ATOM | 14107 | HG12 | VAL | A1178 | 136.159 | 190.327 | 175.442 | 1.00 | 7.81 | H |
| ATOM | 14108 | HG13 | VAL | A1178 | 137.726 | 190.518 | 175.245 | 1.00 | 7.81 | H |
| ATOM | 14109 | HG21 | VAL | A1178 | 137.034 | 191.505 | 172.315 | 1.00 | 7.81 | H |
| ATOM | 14110 | HG22 | VAL | A1178 | 138.402 | 190.762 | 172.634 | 1.00 | 7.81 | H |
| ATOM | 14111 | HG23 | VAL | A1178 | 137.383 | 190.135 | 171.589 | 1.00 | 7.81 | H |
| ATOM | 14112 | N | LEU | A1179 | 135.878 | 187.243 | 175.117 | 1.00 | 6.70 | N |
| ATOM | 14113 | CA | LEU | A1179 | 135.474 | 186.571 | 176.348 | 1.00 | 6.70 | C |
| ATOM | 14114 | C | LEU | A1179 | 136.403 | 185.408 | 176.669 | 1.00 | 6.70 | C |
| ATOM | 14115 | O | LEU | A1179 | 136.751 | 185.182 | 177.834 | 1.00 | 6.70 | O |
| ATOM | 14116 | CB | LEU | A1179 | 134.030 | 186.083 | 176.230 | 1.00 | 6.70 | C |
| ATOM | 14117 | CG | LEU | A1179 | 132.941 | 187.153 | 176.336 | 1.00 | 6.70 | C |
| ATOM | 14118 | CD1 | LEU | A1179 | 131.585 | 186.564 | 175.980 | 1.00 | 6.70 | C |
| ATOM | 14119 | CD2 | LEU | A1179 | 132.911 | 187.764 | 177.729 | 1.00 | 6.70 | C |
| ATOM | 14120 | H | LEU | A1179 | 135.297 | 187.208 | 174.484 | 1.00 | 6.70 | H |
| ATOM | 14121 | HA | LEU | A1179 | 135.522 | 187.203 | 177.083 | 1.00 | 6.70 | H |
| ATOM | 14122 | HB2 | LEU | A1179 | 133.926 | 185.651 | 175.368 | 1.00 | 6.70 | H |
| ATOM | 14123 | HB3 | LEU | A1179 | 133.869 | 185.437 | 176.935 | 1.00 | 6.70 | H |
| ATOM | 14124 | HG | LEU | A1179 | 133.135 | 187.862 | 175.703 | 1.00 | 6.70 | H |
| ATOM | 14125 | HD11 | LEU | A1179 | 130.918 | 187.267 | 176.010 | 1.00 | 6.70 | H |
| ATOM | 14126 | HD12 | LEU | A1179 | 131.630 | 186.187 | 175.087 | 1.00 | 6.70 | H |
| ATOM | 14127 | HD13 | LEU | A1179 | 131.365 | 185.870 | 176.621 | 1.00 | 6.70 | H |
| ATOM | 14128 | HD21 | LEU | A1179 | 132.122 | 188.322 | 177.811 | 1.00 | 6.70 | H |
| ATOM | 14129 | HD22 | LEU | A1179 | 132.882 | 187.051 | 178.386 | 1.00 | 6.70 | H |
| ATOM | 14130 | HD23 | LEU | A1179 | 133.709 | 188.300 | 177.858 | 1.00 | 6.70 | H |
| ATOM | 14131 | N | ILE | A1180 | 136.804 | 184.648 | 175.648 | 1.00 | 6.90 | N |
| ATOM | 14132 | CA | ILE | A1180 | 137.742 | 183.549 | 175.871 | 1.00 | 6.90 | C |
| ATOM | 14133 | C | ILE | A1180 | 139.068 | 184.082 | 176.400 | 1.00 | 6.90 | C |
| ATOM | 14134 | O | ILE | A1180 | 139.663 | 183.509 | 177.322 | 1.00 | 6.90 | O |
| ATOM | 14135 | CB | ILE | A1180 | 137.934 | 182.739 | 174.575 | 1.00 | 6.90 | C |
| ATOM | 14136 | CG1 | ILE | A1180 | 136.653 | 181.978 | 174.232 | 1.00 | 6.90 | C |
| ATOM | 14137 | CG2 | ILE | A1180 | 139.102 | 181.774 | 174.718 | 1.00 | 6.90 | C |
| ATOM | 14138 | CD1 | ILE | A1180 | 136.462 | 181.749 | 172.753 | 1.00 | 6.90 | C |
| ATOM | 14139 | H | ILE | A1180 | 136.553 | 184.746 | 174.831 | 1.00 | 6.90 | H |
| ATOM | 14140 | HA | ILE | A1180 | 137.371 | 182.954 | 176.541 | 1.00 | 6.90 | H |
| ATOM | 14141 | HB | ILE | A1180 | 138.130 | 183.356 | 173.853 | 1.00 | 6.90 | H |
| ATOM | 14142 | HG12 | ILE | A1180 | 136.679 | 181.111 | 174.665 | 1.00 | 6.90 | H |
| ATOM | 14143 | HG13 | ILE | A1180 | 135.890 | 182.483 | 174.554 | 1.00 | 6.90 | H |
| ATOM | 14144 | HG21 | ILE | A1180 | 139.032 | 181.092 | 174.032 | 1.00 | 6.90 | H |
| ATOM | 14145 | HG22 | ILE | A1180 | 139.934 | 182.262 | 174.612 | 1.00 | 6.90 | H |
| ATOM | 14146 | HG23 | ILE | A1180 | 139.067 | 181.363 | 175.596 | 1.00 | 6.90 | H |
| ATOM | 14147 | HD11 | ILE | A1180 | 135.709 | 181.152 | 172.621 | 1.00 | 6.90 | H |
| ATOM | 14148 | HD12 | ILE | A1180 | 136.287 | 182.601 | 172.323 | 1.00 | 6.90 | H |
| ATOM | 14149 | HD13 | ILE | A1180 | 137.268 | 181.352 | 172.389 | 1.00 | 6.90 | H |
| ATOM | 14150 | N | CYS | A1181 | 139.555 | 185.181 | 175.820 | 1.00 | 8.30 | N |
| ATOM | 14151 | CA | CYS | A1181 | 140.802 | 185.772 | 176.300 | 1.00 | 8.30 | C |
| ATOM | 14152 | C | CYS | A1181 | 140.672 | 186.229 | 177.749 | 1.00 | 8.30 | C |
| ATOM | 14153 | O | CYS | A1181 | 141.591 | 186.039 | 178.557 | 1.00 | 8.30 | O |
| ATOM | 14154 | CB | CYS | A1181 | 141.207 | 186.943 | 175.407 | 1.00 | 8.30 | C |
| ATOM | 14155 | SG | CYS | A1181 | 141.647 | 186.475 | 173.719 | 1.00 | 8.30 | S |
| ATOM | 14156 | H | CYS | A1181 | 139.191 | 185.595 | 175.160 | 1.00 | 8.30 | H |
| ATOM | 14157 | HA | CYS | A1181 | 141.505 | 185.104 | 176.259 | 1.00 | 8.30 | H |
| ATOM | 14158 | HB2 | CYS | A1181 | 140.465 | 187.566 | 175.356 | 1.00 | 8.30 | H |
| ATOM | 14159 | HB3 | CYS | A1181 | 141.977 | 187.383 | 175.801 | 1.00 | 8.30 | H |
| ATOM | 14160 | HG | CYS | A1181 | 141.941 | 187.466 | 173.109 | 1.00 | 8.30 | H |
| ATOM | 14161 | N | LEU | A1182 | 139.539 | 186.844 | 178.093 | 1.00 | 5.89 | N |
| ATOM | 14162 | CA | LEU | A1182 | 139.325 | 187.286 | 179.467 | 1.00 | 5.89 | C |
| ATOM | 14163 | C | LEU | A1182 | 139.296 | 186.101 | 180.425 | 1.00 | 5.89 | C |
| ATOM | 14164 | O | LEU | A1182 | 139.847 | 186.170 | 181.530 | 1.00 | 5.89 | O |
| ATOM | 14165 | CB | LEU | A1182 | 138.025 | 188.083 | 179.561 | 1.00 | 5.89 | C |
| ATOM | 14166 | CG | LEU | A1182 | 138.104 | 189.538 | 179.095 | 1.00 | 5.89 | C |
| ATOM | 14167 | CD1 | LEU | A1182 | 136.714 | 190.152 | 179.044 | 1.00 | 5.89 | C |
| ATOM | 14168 | CD2 | LEU | A1182 | 139.021 | 190.350 | 179.997 | 1.00 | 5.89 | C |
| ATOM | 14169 | H | LEU | A1182 | 138.889 | 187.016 | 177.557 | 1.00 | 5.89 | H |
| ATOM | 14170 | HA | LEU | A1182 | 140.058 | 187.865 | 179.728 | 1.00 | 5.89 | H |
| ATOM | 14171 | HB2 | LEU | A1182 | 137.353 | 187.640 | 179.020 | 1.00 | 5.89 | H |
| ATOM | 14172 | HB3 | LEU | A1182 | 137.738 | 188.091 | 180.488 | 1.00 | 5.89 | H |
| ATOM | 14173 | HG | LEU | A1182 | 138.472 | 189.559 | 178.199 | 1.00 | 5.89 | H |
| ATOM | 14174 | HD11 | LEU | A1182 | 136.780 | 191.052 | 178.688 | 1.00 | 5.89 | H |
| ATOM | 14175 | HD12 | LEU | A1182 | 136.152 | 189.609 | 178.469 | 1.00 | 5.89 | H |
| ATOM | 417 | HD13 | LEU | A1182 | 136.346 | 190.176 | 179.941 | 1.00 | 5.89 | H |
| ATOM | 14177 | HD21 | LEU | A1182 | 138.913 | 191.292 | 179.791 | 1.00 | 5.89 | H |
| ATOM | 14178 | HD22 | LEU | A1182 | 138.781 | 190.184 | 180.922 | 1.00 | 5.89 | H |
| ATOM | 14179 | HD23 | LEU | A1182 | 139.939 | 190.081 | 179.841 | 1.00 | 5.89 | H |
| ATOM | 14180 | N | ASN | A1183 | 138.650 | 185.005 | 180.020 | 1.00 | 4.91 | N |
| ATOM | 14181 | CA | ASN | A1183 | 138.626 | 183.815 | 180.865 | 1.00 | 4.91 | C |
| ATOM | 14182 | C | ASN | A1183 | 140.027 | 183.248 | 181.051 | 1.00 | 4.91 | C |
| ATOM | 14183 | O | ASN | A1183 | 140.383 | 182.793 | 182.145 | 1.00 | 4.91 | O |
| ATOM | 14184 | CB | ASN | A1183 | 137.697 | 182.760 | 180.262 | 1.00 | 4.91 | C |
| ATOM | 14185 | CG | ASN | A1183 | 137.281 | 181.679 | 181.266 | 1.00 | 4.91 | C |
| ATOM | 14186 | OD1 | ASN | A1183 | 136.591 | 180.728 | 180.900 | 1.00 | 4.91 | O |
| ATOM | 14187 | ND2 | ASN | A1183 | 137.702 | 181.812 | 182.524 | 1.00 | 4.91 | N |
| ATOM | 14188 | H | ASN | A1183 | 138.229 | 184.928 | 179.274 | 1.00 | 4.91 | H |
| ATOM | 14189 | HA | ASN | A1183 | 138.281 | 184.078 | 181.731 | 1.00 | 4.91 | H |
| ATOM | 14190 | HB2 | ASN | A1183 | 136.895 | 183.198 | 179.937 | 1.00 | 4.91 | H |
| ATOM | 14191 | HB3 | ASN | A1183 | 138.155 | 182.323 | 179.527 | 1.00 | 4.91 | H |
| ATOM | 14192 | HD21 | ASN | A1183 | 137.482 | 181.216 | 183.103 | 1.00 | 4.91 | H |
| ATOM | 14193 | HD22 | ASN | A1183 | 138.186 | 182.477 | 182.768 | 1.00 | 4.91 | H |
| ATOM | 14194 | N | MET | A1184 | 140.836 | 183.256 | 179.990 | 1.00 | 4.33 | N |
| ATOM | 14195 | CA | MET | A1184 | 142.220 | 182.808 | 180.117 | 1.00 | 4.33 | C |
| ATOM | 14196 | C | MET | A1184 | 142.990 | 183.683 | 181.098 | 1.00 | 4.33 | C |
| ATOM | 14197 | O | MET | A1184 | 143.746 | 183.178 | 181.939 | 1.00 | 4.33 | O |
| ATOM | 14198 | CB | MET | A1184 | 142.900 | 182.817 | 178.749 | 1.00 | 4.33 | C |
| ATOM | 14199 | CG | MET | A1184 | 143.789 | 181.617 | 178.498 | 1.00 | 4.33 | C |
| ATOM | 14200 | SD | MET | A1184 | 144.439 | 181.580 | 176.818 | 1.00 | 4.33 | S |
| ATOM | 14201 | CE | MET | A1184 | 146.109 | 181.006 | 177.114 | 1.00 | 4.33 | C |
| ATOM | 14202 | H | MET | A1184 | 140.611 | 183.511 | 179.200 | 1.00 | 4.33 | H |
| ATOM | 14203 | HA | MET | A1184 | 142.223 | 181.895 | 180.445 | 1.00 | 4.33 | H |
| ATOM | 14204 | HB2 | MET | A1184 | 142.219 | 182.828 | 178.059 | 1.00 | 4.33 | H |
| ATOM | 14205 | HB3 | MET | A1184 | 143.450 | 183.612 | 178.679 | 1.00 | 4.33 | H |
| ATOM | 14206 | HG2 | MET | A1184 | 144.541 | 181.648 | 179.110 | 1.00 | 4.33 | H |
| ATOM | 14207 | HG3 | MET | A1184 | 143.276 | 180.809 | 178.646 | 1.00 | 4.33 | H |
| ATOM | 14208 | HE1 | MET | A1184 | 146.575 | 180.940 | 176.266 | 1.00 | 4.33 | H |
| ATOM | 14209 | HE2 | MET | A1184 | 146.562 | 181.639 | 177.693 | 1.00 | 4.33 | H |
| ATOM | 14210 | HE3 | MET | A1184 | 146.071 | 180.136 | 177.540 | 1.00 | 4.33 | H |
| ATOM | 14211 | N | VAL | A1185 | 142.809 | 185.002 | 181.007 | 1.00 | 5.28 | N |
| ATOM | 14212 | CA | VAL | A1185 | 143.482 | 185.905 | 181.936 | 1.00 | 5.28 | C |
| ATOM | 14213 | C | VAL | A1185 | 143.052 | 185.603 | 183.365 | 1.00 | 5.28 | C |
| ATOM | 14214 | O | VAL | A1185 | 143.874 | 185.594 | 184.290 | 1.00 | 5.28 | O |
| ATOM | 14215 | CB | VAL | A1185 | 143.203 | 187.371 | 181.557 | 1.00 | 5.28 | C |
| ATOM | 14216 | CG1 | VAL | A1185 | 143.555 | 188.299 | 182.713 | 1.00 | 5.28 | C |
| ATOM | 14217 | CG2 | VAL | A1185 | 143.978 | 187.751 | 180.306 | 1.00 | 5.28 | C |
| ATOM | 14218 | H | VAL | A1185 | 142.309 | 185.392 | 180.426 | 1.00 | 5.28 | H |
| ATOM | 14219 | HA | VAL | A1185 | 144.440 | 185.760 | 181.877 | 1.00 | 5.28 | H |
| ATOM | 14220 | HB | VAL | A1185 | 142.257 | 187.473 | 181.367 | 1.00 | 5.28 | H |
| ATOM | 14221 | HG11 | VAL | A1185 | 143.646 | 189.203 | 182.373 | 1.00 | 5.28 | H |
| ATOM | 14222 | HG12 | VAL | A1185 | 142.848 | 188.268 | 183.376 | 1.00 | 5.28 | H |
| ATOM | 14223 | HG13 | VAL | A1185 | 144.394 | 188.010 | 183.105 | 1.00 | 5.28 | H |
| ATOM | 14224 | HG21 | VAL | A1185 | 143.792 | 188.678 | 180.089 | 1.00 | 5.28 | H |
| ATOM | 14225 | HG22 | VAL | A1185 | 144.926 | 187.635 | 180.475 | 1.00 | 5.28 | H |
| ATOM | 14226 | HG23 | VAL | A1185 | 143.700 | 187.178 | 179.575 | 1.00 | 5.28 | H |
| ATOM | 14227 | N | THR | A1186 | 141.758 | 185.350 | 183.568 | 1.00 | 4.79 | N |
| ATOM | .4228 | CA | THR | A1186 | 141.277 | 184.983 | 184.897 | 1.00 | 4.79 | C |
| ATOM | 14229 | C | THR | A1186 | 141.941 | 183.701 | 185.382 | 1.00 | 4.79 | C |
| ATOM | 14230 | o | THR | A1186 | 142.314 | 183.590 | 186.556 | 1.00 | 4.79 | o |
| ATOM | 14231 | CB | THR | A1186 | 139.756 | 184.821 | 184.880 | 1.00 | 4.79 | C |
| ATOM | 14232 | OG1 | THR | A1186 | 139.150 | 186.002 | 184.341 | 1.00 | 4.79 | O |
| ATOM | 14233 | CG2 | THR | A1186 | 139.223 | 184.573 | 186.287 | 1.00 | 4.79 | C |
| ATOM | 14234 | H | THR | A1186 | 141.148 | 185.383 | 182.963 | 1.00 | 4.79 | H |
| ATOM | 14235 | HA | THR | A1186 | 141.498 | 185.692 | 185.521 | 1.00 | 4.79 | H |
| ATOM | 14236 | HB | THR | A1186 | 139.521 | 184.059 | 184.327 | 1.00 | 4.79 | H |
| ATOM | 14237 | HG1 | THR | A1186 | 139.346 | 186.667 | 184.815 | 1.00 | 4.79 | H |
| ATOM | 14238 | HG21 | THR | A1186 | 138.254 | 184.595 | 186.281 | 1.00 | 4.79 | H |
| ATOM | 14239 | HG22 | THR | A1186 | 139.515 | 183.706 | 186.608 | 1.00 | 4.79 | H |
| ATOM | 14240 | HG23 | THR | A1186 | 139.549 | 185.260 | 186.890 | 1.00 | 4.79 | H |
| ATOM | 14241 | N | MET | A1187 | 142.093 | 182.719 | 184.492 | 1.00 | 4.25 | N |
| ATOM | 14242 | CA | MET | A1187 | 142.759 | 181.477 | 184.871 | 1.00 | 4.25 | C |
| ATOM | 14243 | C | MET | A1187 | 144.206 | 181.731 | 185.274 | 1.00 | 4.25 | C |
| ATOM | 14244 | O | MET | A1187 | 144.735 | 181.066 | 186.172 | 1.00 | 4.25 | O |
| ATOM | 14245 | CB | MET | A1187 | 142.699 | 180.475 | 183.718 | 1.00 | 4.25 | C |
| ATOM | 14246 | CG | MET | A1187 | 141.366 | 179.764 | 183.581 | 1.00 | 4.25 | C |
| ATOM | 14247 | SD | MET | A1187 | 141.511 | 178.217 | 182.668 | 1.00 | 4.25 | S |
| ATOM | 14248 | CE | MET | A1187 | 139.921 | 178.161 | 181.847 | 1.00 | 4.25 | C |
| ATOM | 14249 | H | MET | A1187 | 141.823 | 182.747 | 183.676 | 1.00 | 4.25 | H |
| ATOM | 14250 | HA | MET | A1187 | 142.289 | 181.091 | 185.625 | 1.00 | 4.25 | H |
| ATOM | 14251 | HB2 | MET | A1187 | 142.870 | 180.946 | 182.888 | 1.00 | 4.25 | H |
| ATOM | 14252 | HB3 | MET | A1187 | 143.382 | 179.800 | 183.858 | 1.00 | 4.25 | H |
| ATOM | 14253 | HG2 | MET | A1187 | 141.021 | 179.561 | 184.465 | 1.00 | 4.25 | H |
| ATOM | 14254 | HG3 | MET | A1187 | 140.747 | 180.339 | 183.105 | 1.00 | 4.25 | H |
| ATOM | 14255 | HE1 | MET | A1187 | 139.874 | 177.359 | 181.304 | 1.00 | 4.25 | H |
| ATOM | 14256 | HE2 | MET | A1187 | 139.221 | 178.150 | 182.518 | 1.00 | 4.25 | H |
| ATOM | 14257 | HE3 | MET | A1187 | 139.829 | 178.947 | 181.287 | 1.00 | 4.25 | H |
| ATOM | 14258 | N | MET | A1188 | 144.867 | 182.685 | 184.614 | 1.00 | 3.50 | N |
| ATOM | 14259 | CA | MET | A1188 | 146.268 | 182.963 | 184.916 | 1.00 | 3.50 | C |
| ATOM | 14260 | C | MET | A1188 | 146.471 | 183.523 | 186.321 | 1.00 | 3.50 | C |
| ATOM | 14261 | O | MET | A1188 | 147.596 | 183.485 | 186.830 | 1.00 | 3.50 | O |
| ATOM | 14262 | CB | MET | A1188 | 146.839 | 183.943 | 183.891 | 1.00 | 3.50 | C |
| ATOM | 14263 | CG | MET | A1188 | 147.138 | 183.318 | 182.539 | 1.00 | 3.50 | C |
| ATOM | 14264 | SD | MET | A1188 | 147.488 | 184.545 | 181.266 | 1.00 | 3.50 | S |
| ATOM | 14265 | CE | MET | A1188 | 147.467 | 183.523 | 179.795 | 1.00 | 3.50 | C |
| ATOM | 14266 | H | MET | A1188 | 144.531 | 183.177 | 183.994 | 1.00 | 3.50 | H |
| ATOM | 14267 | HA | MET | A1188 | 146.769 | 182.136 | 184.840 | 1.00 | 3.50 | H |
| ATOM | 14268 | HB2 | MET | A1188 | 146.198 | 184.657 | 183.751 | 1.00 | 3.50 | H |
| ATOM | 14269 | HB3 | MET | A1188 | 147.668 | 184.309 | 184.237 | 1.00 | 3.50 | H |
| ATOM | 14270 | HG2 | MET | A1188 | 147.913 | 182.742 | 182.622 | 1.00 | 3.50 | H |
| ATOM | 14271 | HG3 | MET | A1188 | 146.369 | 182.800 | 182.254 | 1.00 | 3.50 | H |
| ATOM | 14272 | HE1 | MET | A1188 | 147.633 | 184.083 | 179.020 | 1.00 | 3.50 | H |
| ATOM | 14273 | HE2 | MET | A1188 | 148.159 | 182.848 | 179.870 | 1.00 | 3.50 | H |
| ATOM | 14274 | HE3 | MET | A1188 | 146.597 | 183.100 | 179.717 | 1.00 | 3.50 | H |
| ATOM | 14275 | N | VAL | A1189 | 145.418 | 184.042 | 186.958 | 1.00 | 4.57 | N |
| ATOM | 14276 | CA | VAL | A1189 | 145.579 | 184.694 | 188.254 | 1.00 | 4.57 | C |
| ATOM | 14277 | C | VAL | A1189 | 145.800 | 183.691 | 189.381 | 1.00 | 4.57 | C |
| ATOM | 14278 | O | VAL | A1189 | 146.350 | 184.054 | 190.428 | 1.00 | 4.57 | O |
| ATOM | 14279 | CB | VAL | A1189 | 144.354 | 185.581 | 188.551 | 1.00 | 4.57 | C |
| ATOM | 14280 | CG1 | VAL | A1189 | 144.349 | 186.032 | 190.007 | 1.00 | 4.57 | C |
| ATOM | 14281 | CG2 | VAL | A1189 | 144.334 | 186.784 | 187.620 | 1.00 | 4.57 | C |
| ATOM | 14282 | H | VAL | A1189 | 144.610 | 184.029 | 186.664 | 1.00 | 4.57 | H |
| ATOM | 14283 | HA | VAL | A1189 | 146.358 | 185.270 | 188.217 | 1.00 | 4.57 | H |
| ATOM | 14284 | HB | VAL | A1189 | 143.547 | 185.067 | 188.394 | 1.00 | 4.57 | H |
| ATOM | 14285 | HG11 | VAL | A1189 | 143.755 | 186.793 | 190.097 | 1.00 | 4.57 | H |
| ATOM | 14286 | HG12 | VAL | A1189 | 144.036 | 185.304 | 190.567 | 1.00 | 4.57 | H |
| ATOM | 14287 | HG13 | VAL | A1189 | 145.249 | 186.286 | 190.262 | 1.00 | 4.57 | H |
| ATOM | 14288 | HG21 | VAL | A1189 | 143.559 | 187.329 | 187.826 | 1.00 | 4.57 | H |
| ATOM | 14289 | HG22 | VAL | A1189 | 145.146 | 187.298 | 187.751 | 1.00 | 4.57 | H |
| ATOM | 14290 | HG23 | VAL | A1189 | 144.284 | 186.471 | 186.703 | 1.00 | 4.57 | H |
| ATOM | 14291 | N | GLU | A1190 | 145.395 | 182.438 | 189.198 | 1.00 | 4.10 | N |
| ATOM | 14292 | CA | GLU | A1190 | 145.502 | 181.451 | 190.265 | 1.00 | 4.10 | C |
| ATOM | 14293 | C | GLU | A1190 | 146.962 | 181.205 | 190.629 | 1.00 | 4.10 | C |
| ATOM | 14294 | O | GLU | A1190 | 147.824 | 181.074 | 189.755 | 1.00 | 4.10 | O |
| ATOM | 14295 | CB | GLU | A1190 | 144.837 | 180.144 | 189.835 | 1.00 | 4.10 | C |
| ATOM | 14296 | CG | GLU | A1190 | 144.684 | 179.119 | 190.944 | 1.00 | 4.10 | C |
| ATOM | 14297 | CD | GLU | A1190 | 144.294 | 177.751 | 190.418 | 1.00 | 4.10 | C |
| ATOM | 14298 | OE1 | GLU | A1190 | 143.296 | 177.664 | 189.672 | 1.00 | 4.10 | O |
| ATOM | 14299 | OE2 | GLU | A1190 | 144.983 | 176.764 | 190.748 | 1.00 | 4.10 | O |
| ATOM | 14300 | H | GLU | A1190 | 145.054 | 182.135 | 188.469 | 1.00 | 4.10 | H |
| ATOM | 14301 | HA | GLU | A1190 | 145.044 | 181.781 | 191.054 | 1.00 | 4.10 | H |
| ATOM | 14302 | HB2 | GLU | A1190 | 143.950 | 180.345 | 189.496 | 1.00 | 4.10 | H |
| ATOM | 14303 | HB3 | GLU | A1190 | 145.371 | 179.740 | 189.133 | 1.00 | 4.10 | H |
| ATOM | 14304 | HG2 | GLU | A1190 | 145.525 | 179.034 | 191.417 | 1.00 | 4.10 | H |
| ATOM | 14305 | HG3 | GLU | A1190 | 143.990 | 179.415 | 191.554 | 1.00 | 4.10 | H |
| ATOM | 14306 | N | LYS | A1191 | 147.238 | 181.140 | 191.931 | 1.00 | 4.84 | N |
| ATOM | 14307 | CA | LYS | A1191 | 148.595 | 180.930 | 192.414 | 1.00 | 4.84 | C |
| ATOM | 14308 | C | LYS | A1191 | 148.553 | 180.179 | 193.737 | 1.00 | 4.84 | C |
| ATOM | 14309 | O | LYS | A1191 | 147.526 | 180.133 | 194.419 | 1.00 | 4.84 | O |
| ATOM | 14310 | CB | LYS | A1191 | 149.346 | 182.256 | 192.585 | 1.00 | 4.84 | C |
| ATOM | 14311 | CG | LYS | A1191 | 148.888 | 183.078 | 193.782 | 1.00 | 4.84 | C |
| ATOM | 14312 | CD | LYS | A1191 | 149.101 | 184.577 | 193.581 | 1.00 | 4.84 | C |
| ATOM | 14313 | CE | LYS | A1191 | 150.448 | 184.899 | 192.941 | 1.00 | 4.84 | C |
| ATOM | 14314 | NZ | LYS | A1191 | 151.585 | 184.300 | 193.693 | 1.00 | 4.84 | N |
| ATOM | 14315 | H | LYS | A1191 | 146.652 | 181.217 | 192.555 | 1.00 | 4.84 | H |
| ATOM | 14316 | HA | LYS | A1191 | 149.082 | 180.388 | 191.774 | 1.00 | 4.84 | H |
| ATOM | 14317 | HB2 | LYS | A1191 | 150.291 | 182.069 | 192.694 | 1.00 | 4.84 | H |
| ATOM | 14318 | HB3 | LYS | A1191 | 149.205 | 182.789 | 191.787 | 1.00 | 4.84 | H |
| ATOM | 14319 | HG2 | LYS | A1191 | 147.941 | 182.926 | 193.928 | 1.00 | 4.84 | H |
| ATOM | 14320 | HG3 | LYS | A1191 | 149.393 | 182.806 | 194.564 | 1.00 | 4.84 | H |
| ATOM | 14321 | HD2 | LYS | A1191 | 148.403 | 184.921 | 193.002 | 1.00 | 4.84 | H |
| ATOM | 14322 | HD3 | LYS | A1191 | 149.065 | 185.020 | 194.443 | 1.00 | 4.84 | H |
| ATOM | 14323 | HE2 | LYS | A1191 | 150.471 | 184.566 | 192.031 | 1.00 | 4.84 | H |
| ATOM | 14324 | HE3 | LYS | A1191 | 150.569 | 185.861 | 192.940 | 1.00 | 4.84 | H |
| ATOM | 14325 | HZ1 | LYS | A1191 | 152.352 | 184.477 | 193.277 | 1.00 | 4.84 | H |
| ATOM | 14326 | HZ2 | LYS | A1191 | 151.618 | 184.640 | 194.515 | 1.00 | 4.84 | H |
| ATOM | 14327 | HZ3 | LYS | A1191 | 151.484 | 183.418 | 193.749 | 1.00 | 4.84 | H |
| ATOM | 14328 | N | GLU | A1192 | 149.692 | 179.588 | 194.089 | 1.00 | 5.92 | N |
| ATOM | 14329 | CA | GLU | A1192 | 149.803 | 178.842 | 195.335 | 1.00 | 5.92 | C |
| ATOM | 14330 | C | GLU | A1192 | 149.748 | 179.781 | 196.533 | 1.00 | 5.92 | C |
| ATOM | 14331 | O | GLU | A1192 | 150.279 | 180.895 | 196.497 | 1.00 | 5.92 | O |
| ATOM | 14332 | CB | GLU | A1192 | 151.107 | 178.044 | 195.359 | 1.00 | 5.92 | C |
| ATOM | 14333 | CG | GLU | A1192 | 151.145 | 176.942 | 196.413 | 1.00 | 5.92 | C |
| ATOM | 14334 | CD | GLU | A1192 | 152.094 | 177.247 | 197.560 | 1.00 | 5.92 | C |
| ATOM | 14335 | OE1 | GLU | A1192 | 152.441 | 178.431 | 197.757 | 1.00 | 5.92 | O |
| ATOM | 14336 | OE2 | GLU | A1192 | 152.495 | 176.299 | 198.267 | 1.00 | 5.92 | O |
| ATOM | 14337 | H | GLU | A1192 | 150.415 | 179.605 | 193.623 | 1.00 | 5.92 | H |
| ATOM | 14338 | HA | GLU | A1192 | 149.062 | 178.219 | 195.402 | 1.00 | 5.92 | H |
| ATOM | 14339 | HB2 | GLU | A1192 | 151.231 | 177.628 | 194.493 | 1.00 | 5.92 | H |
| ATOM | 14340 | HB3 | GLU | A1192 | 151.840 | 178.654 | 195.531 | 1.00 | 5.92 | H |
| ATOM | 14341 | HG2 | GLU | A1192 | 150.256 | 176.828 | 196.783 | 1.00 | 5.92 | H |
| ATOM | 14342 | HG3 | GLU | A1192 | 151.440 | 176.119 | 195.994 | 1.00 | 5.92 | H |
| ATOM | 14343 | N | GLY | A1193 | 149.103 | 179.321 | 197.603 | 1.00 | 6.35 | N |
| ATOM | 14344 | CA | GLY | A1193 | 149.020 | 180.074 | 198.835 | 1.00 | 6.35 | C |
| ATOM | 14345 | C | GLY | A1193 | 147.904 | 181.093 | 198.898 | 1.00 | 6.35 | C |
| ATOM | 14346 | O | GLY | A1193 | 147.751 | 181.753 | 199.934 | 1.00 | 6.35 | O |
| ATOM | 14347 | H | GLY | A1193 | 148.699 | 178.562 | 197.632 | 1.00 | 6.35 | H |
| ATOM | 14348 | HA2 | GLY | A1193 | 148.901 | 179.456 | 199.573 | 1.00 | 6.35 | H |
| ATOM | 14349 | HA3 | GLY | A1193 | 149.858 | 180.544 | 198.972 | 1.00 | 6.35 | H |
| ATOM | 14350 | N | GLN | A1194 | 147.119 | 181.244 | 197.836 | 1.00 | 6.28 | N |
| ATOM | 14351 | CA | GLN | A1194 | 146.029 | 182.207 | 197.851 | 1.00 | 6.28 | C |
| ATOM | 14352 | C | GLN | A1194 | 144.952 | 181.780 | 198.844 | 1.00 | 6.28 | C |
| ATOM | 14353 | O | GLN | A1194 | 144.753 | 180.591 | 199.109 | 1.00 | 6.28 | O |
| ATOM | 14354 | CB | GLN | A1194 | 145.431 | 182.358 | 196.452 | 1.00 | 6.28 | C |
| ATOM | 14355 | CG | GLN | A1194 | 144.404 | 181.297 | 196.090 | 1.00 | 6.28 | C |
| ATOM | 14356 | CD | GLN | A1194 | 144.168 | 181.201 | 194.594 | 1.00 | 6.28 | C |
| ATOM | 14357 | OE1 | GLN | A1194 | 144.854 | 181.845 | 193.801 | 1.00 | 6.28 | O |
| ATOM | 14358 | NE2 | GLN | A1194 | 143.191 | 180.392 | 194.202 | 1.00 | 6.28 | N |
| ATOM | 14359 | H | GLN | A1194 | 147.198 | 180.806 | 197.100 | 1.00 | 6.28 | H |
| ATOM | 14360 | HA | GLN | A1194 | 146.369 | 183.071 | 198.131 | 1.00 | 6.28 | H |
| ATOM | 14361 | HB2 | GLN | A1194 | 144.994 | 183.222 | 196.393 | 1.00 | 6.28 | H |
| ATOM | 14362 | HB3 | GLN | A1194 | 146.149 | 182.310 | 195.803 | 1.00 | 6.28 | H |
| ATOM | 14363 | HG2 | GLN | A1194 | 144.717 | 180.433 | 196.401 | 1.00 | 6.28 | H |
| ATOM | 14364 | HG3 | GLN | A1194 | 143.558 | 181.516 | 196.511 | 1.00 | 6.28 | H |
| ATOM | 14365 | HE21 | GLN | A1194 | 142.732 | 179.958 | 194.786 | 1.00 | 6.28 | H |
| ATOM | 14366 | HE22 | GLN | A1194 | 143.017 | 180.302 | 193.365 | 1.00 | 6.28 | H |
| ATOM | 14367 | N | SER | A1195 | 144.259 | 182.769 | 199.399 | 1.00 | 9.34 | N |
| ATOM | 14368 | CA | SER | A1195 | 143.211 | 182.496 | 200.369 | 1.00 | 9.34 | C |
| ATOM | 14369 | C | SER | A1195 | 142.035 | 181.789 | 199.704 | 1.00 | 9.34 | C |
| ATOM | 14370 | O | SER | A1195 | 141.815 | 181.899 | 198.495 | 1.00 | 9.34 | O |
| ATOM | 14371 | CB | SER | A1195 | 142.738 | 183.795 | 201.022 | 1.00 | 9.34 | C |
| ATOM | 14372 | OG | SER | A1195 | 141.955 | 184.562 | 200.124 | 1.00 | 9.34 | O |
| ATOM | 14373 | H | SER | A1195 | 144.377 | 183.604 | 199.228 | 1.00 | 9.34 | H |
| ATOM | 14374 | HA | SER | A1195 | 143.561 | 181.916 | 201.063 | 1.00 | 9.34 | H |
| ATOM | 14375 | HB2 | SER | A1195 | 142.204 | 183.581 | 201.802 | 1.00 | 9.34 | H |
| ATOM | 14376 | HB3 | SER | A1195 | 143.515 | 184.313 | 201.284 | 1.00 | 9.34 | H |
| ATOM | 14377 | HG | SER | A1195 | 141.720 | 185.281 | 200.490 | 1.00 | 9.34 | H |
| ATOM | 14378 | N | GLN | A1196 | 141.280 | 181.042 | 200.513 | 1.00 | 11.80 | N |
| ATOM | 14379 | CA | GLN | A1196 | 140.120 | 180.324 | 199.991 | 1.00 | 11.80 | C |
| ATOM | 14380 | C | GLN | A1196 | 139.088 | 181.277 | 199.395 | 1.00 | 11.80 | C |
| ATOM | 14381 | O | GLN | A1196 | 138.363 | 180.905 | 198.463 | 1.00 | 11.80 | O |
| ATOM | 14382 | CB | GLN | A1196 | 139.483 | 179.459 | 201.088 | 1.00 | 11.80 | C |
| ATOM | 14383 | CG | GLN | A1196 | 138.835 | 180.195 | 202.274 | 1.00 | 11.80 | C |
| ATOM | 14384 | CD | GLN | A1196 | 139.674 | 181.324 | 202.837 | 1.00 | 11.80 | C |
| ATOM | 14385 | OE1 | GLN | A1196 | 140.795 | 181.109 | 203.298 | 1.00 | 11.80 | O |
| ATOM | 14386 | NE2 | GLN | A1196 | 139.136 | 182.536 | 202.802 | 1.00 | 11.80 | N |
| ATOM | 14387 | H | GLN | A1196 | 141.433 | 180.926 | 201.350 | 1.00 | 11.80 | H |
| ATOM | 14388 | HA | GLN | A1196 | 140.416 | 179.731 | 199.283 | 1.00 | 11.80 | H |
| ATOM | 14389 | HB2 | GLN | A1196 | 138.796 | 178.910 | 200.682 | 1.00 | 11.80 | H |
| ATOM | 14390 | HB3 | GLN | A1196 | 140.175 | 178.886 | 201.455 | 1.00 | 11.80 | H |
| ATOM | 14391 | HG2 | GLN | A1196 | 137.988 | 180.568 | 201.987 | 1.00 | 11.80 | H |
| ATOM | 14392 | HG3 | GLN | A1196 | 138.686 | 179.556 | 202.989 | 1.00 | 11.80 | H |
| ATOM | 14393 | HE21 | GLN | A1196 | 138.351 | 182.646 | 202.468 | 1.00 | 11.80 | H |
| ATOM | 14394 | HE22 | GLN | A1196 | 139.573 | 183.211 | 203.106 | 1.00 | 11.80 | H |
| ATOM | 14395 | N | HIS | A1197 | 139.010 | 182.508 | 199.909 | 1.00 | 8.61 | N |
| ATOM | 14396 | CA | HIS | A1197 | 138.066 | 183.481 | 199.365 | 1.00 | 8.61 | C |
| ATOM | 14397 | C | HIS | A1197 | 138.387 | 183.793 | 197.908 | 1.00 | 8.61 | C |
| ATOM | 14398 | O | HIS | A1197 | 137.484 | 183.874 | 197.064 | 1.00 | 8.61 | O |
| ATOM | 14399 | CB | HIS | A1197 | 138.094 | 184.753 | 200.216 | 1.00 | 8.61 | C |
| ATOM | 14400 | CG | HIS | A1197 | 137.290 | 185.884 | 199.652 | 1.00 | 8.61 | C |
| ATOM | 14401 | ND1 | HIS | A1197 | 135.918 | 185.835 | 199.528 | 1.00 | 8.61 | N |
| ATOM | 14402 | CD2 | HIS | A1197 | 137.666 | 187.098 | 199.185 | 1.00 | 8.61 | C |
| ATOM | 14403 | CE1 | HIS | A1197 | 135.484 | 186.970 | 199.009 | 1.00 | 8.61 | C |
| ATOM | 14404 | NE2 | HIS | A1197 | 136.525 | 187.753 | 198.791 | 1.00 | 8.61 | N |
| ATOM | 14405 | H | HIS | A1197 | 139.492 | 182.803 | 200.555 | 1.00 | 8.61 | H |
| ATOM | 14406 | HA | HIS | A1197 | 137.171 | 183.112 | 199.404 | 1.00 | 8.61 | H |
| ATOM | 14407 | HB2 | HIS | A1197 | 137.738 | 184.549 | 201.095 | 1.00 | 8.61 | H |
| ATOM | 14408 | HB3 | HIS | A1197 | 139.012 | 185.053 | 200.298 | 1.00 | 8.61 | H |
| ATOM | 14409 | HD1 | HIS | A1197 | 135.422 | 185.171 | 199.755 | 1.00 | 8.61 | H |
| ATOM | 14410 | HD2 | HIS | A1197 | 138.535 | 187.426 | 199.139 | 1.00 | 8.61 | H |
| ATOM | 14411 | HE1 | HIS | A1197 | 134.597 | 187.181 | 198.826 | 1.00 | 8.61 | H |
| ATOM | 14412 | N | MET | A1198 | 139.674 | 183.962 | 197.594 | 1.00 | 11.33 | N |
| ATOM | 14413 | CA | MET | A1198 | 140.077 | 184.199 | 196.212 | 1.00 | 11.33 | C |
| ATOM | 14414 | C | MET | A1198 | 139.711 | 183.012 | 195.331 | 1.00 | 11.33 | C |
| ATOM | 14415 | O | MET | A1198 | 139.263 | 183.185 | 194.191 | 1.00 | 11.33 | O |
| ATOM | 14416 | CB | MET | A1198 | 141.581 | 184.474 | 196.147 | 1.00 | 11.33 | C |
| ATOM | 14417 | CG | MET | A1198 | 142.089 | 184.862 | 194.766 | 1.00 | 11.33 | C |
| ATOM | 14418 | SD | MET | A1198 | 141.586 | 186.524 | 194.280 | 1.00 | 11.33 | S |
| ATOM | 14419 | CE | MET | A1198 | 142.860 | 186.930 | 193.089 | 1.00 | 11.33 | C |
| ATOM | 14420 | H | MET | A1198 | 140.324 | 183.938 | 198.158 | 1.00 | 11.33 | H |
| ATOM | 14421 | HA | MET | A1198 | 139.617 | 184.987 | 195.883 | 1.00 | 11.33 | H |
| ATOM | 14422 | HB2 | MET | A1198 | 141.793 | 185.199 | 196.756 | 1.00 | 11.33 | H |
| ATOM | 14423 | HB3 | MET | A1198 | 142.053 | 183.671 | 196.418 | 1.00 | 11.33 | H |
| ATOM | 14424 | HG2 | MET | A1198 | 143.059 | 184.835 | 194.770 | 1.00 | 11.33 | H |
| ATOM | 14425 | HG3 | MET | A1198 | 141.744 | 184.239 | 194.109 | 1.00 | 11.33 | H |
| ATOM | 14426 | HE1 | MET | A1198 | 142.692 | 187.820 | 192.740 | 1.00 | 11.33 | H |
| ATOM | 14427 | HE2 | MET | A1198 | 143.724 | 186.904 | 193.528 | 1.00 | 11.33 | H |
| ATOM | 14428 | HE3 | MET | A1198 | 142.834 | 186.282 | 192.368 | 1.00 | 11.33 | H |
| ATOM | 14429 | N | THR | A1199 | 139.902 | 181.795 | 195.844 | 1.00 | 7.76 | N |
| ATOM | 14430 | CA | THR | A1199 | 139.513 | 180.603 | 195.099 | 1.00 | 7.76 | C |
| ATOM | 14431 | C | THR | A1199 | 138.023 | 180.627 | 194.782 | 1.00 | 7.76 | C |
| ATOM | 14432 | O | THR | A1199 | 137.607 | 180.318 | 193.658 | 1.00 | 7.76 | O |
| ATOM | 14433 | CB | THR | A1199 | 139.872 | 179.348 | 195.898 | 1.00 | 7.76 | C |
| ATOM | 14434 | OG1 | THR | A1199 | 141.270 | 179.362 | 196.215 | 1.00 | 7.76 | O |
| ATOM | 14435 | CG2 | THR | A1199 | 139.548 | 178.088 | 195.105 | 1.00 | 7.76 | C |
| ATOM | 14436 | H | THR | A1199 | 140.253 | 181.636 | 196.614 | 1.00 | 7.76 | H |
| ATOM | 14437 | HA | THR | A1199 | 139.999 | 180.579 | 194.260 | 1.00 | 7.76 | H |
| ATOM | 14438 | HB | THR | A1199 | 139.357 | 179.333 | 196.720 | 1.00 | 7.76 | H |
| ATOM | 14439 | HG1 | THR | A1199 | 141.465 | 178.690 | 196.680 | 1.00 | 7.76 | H |
| ATOM | 14440 | HG21 | THR | A1199 | 139.885 | 177.309 | 195.574 | 1.00 | 7.76 | H |
| ATOM | 14441 | HG22 | THR | A1199 | 138.589 | 177.998 | 194.997 | 1.00 | 7.76 | H |
| ATOM | 14442 | HG23 | THR | A1199 | 139.963 | 178.131 | 194.230 | 1.00 | 7.76 | H |
| ATOM | 14443 | N | GLU | A1200 | 137.201 | 180.995 | 195.768 | 1.00 | 9.58 | N |
| ATOM | 14444 | CA | GLU | A1200 | 135.760 | 181.051 | 195.537 | 1.00 | 9.58 | C |
| ATOM | 14445 | C | GLU | A1200 | 135.408 | 182.096 | 194.486 | 1.00 | 9.58 | C |
| ATOM | 14446 | O | GLU | A1200 | 134.570 | 181.848 | 193.610 | 1.00 | 9.58 | O |
| ATOM | 14447 | CB | GLU | A1200 | 135.028 | 181.339 | 196.848 | 1.00 | 9.58 | C |
| ATOM | 14448 | CG | GLU | A1200 | 135.188 | 180.251 | 197.895 | 1.00 | 9.58 | C |
| ATOM | 14449 | CD | GLU | A1200 | 134.883 | 178.869 | 197.350 | 1.00 | 9.58 | C |
| ATOM | 14450 | OE1 | GLU | A1200 | 133.709 | 178.610 | 197.012 | 1.00 | 9.58 | O |
| ATOM | 14451 | OE2 | GLU | A1200 | 135.818 | 178.047 | 197.250 | 1.00 | 9.58 | O |
| ATOM | 14452 | H | GLU | A1200 | 137.448 | 181.214 | 196.563 | 1.00 | 9.58 | H |
| ATOM | 14453 | HA | GLU | A1200 | 135.463 | 180.193 | 195.198 | 1.00 | 9.58 | H |
| ATOM | 14454 | HB2 | GLU | A1200 | 135.371 | 182.165 | 197.225 | 1.00 | 9.58 | H |
| ATOM | 14455 | HB3 | GLU | A1200 | 134.080 | 181.436 | 196.662 | 1.00 | 9.58 | H |
| ATOM | 14456 | HG2 | GLU | A1200 | 136.104 | 180.249 | 198.213 | 1.00 | 9.58 | H |
| ATOM | 14457 | HG3 | GLU | A1200 | 134.582 | 180.428 | 198.631 | 1.00 | 9.58 | H |
| ATOM | 14458 | N | VAL | A1201 | 136.035 | 183.273 | 194.554 | 1.00 | 6.93 | N |
| ATOM | 14459 | CA | VAL | A1201 | 135.735 | 184.312 | 193.568 | 1.00 | 6.93 | C |
| ATOM | 14460 | C | VAL | A1201 | 136.112 | 183.837 | 192.170 | 1.00 | 6.93 | C |
| ATOM | 14461 | O | VAL | A1201 | 135.361 | 184.035 | 191.205 | 1.00 | 6.93 | O |
| ATOM | 14462 | CB | VAL | A1201 | 136.440 | 185.639 | 193.912 | 1.00 | 6.93 | C |
| ATOM | 14463 | CG1 | VAL | A1201 | 135.731 | 186.802 | 193.236 | 1.00 | 6.93 | C |
| ATOM | 14464 | CG2 | VAL | A1201 | 136.485 | 185.877 | 195.410 | 1.00 | 6.93 | C |
| ATOM | 14465 | H | VAL | A1201 | 136.627 | 183.479 | 195.142 | 1.00 | 6.93 | H |
| ATOM | 14466 | HA | VAL | A1201 | 134.779 | 184.478 | 193.574 | 1.00 | 6.93 | H |
| ATOM | 14467 | HB | VAL | A1201 | 137.352 | 185.608 | 193.583 | 1.00 | 6.93 | H |
| ATOM | 14468 | HG11 | VAL | A1201 | 136.205 | 187.623 | 193.440 | 1.00 | 6.93 | H |
| ATOM | 14469 | HG12 | VAL | A1201 | 135.729 | 186.650 | 192.278 | 1.00 | 6.93 | H |
| ATOM | 14470 | HG13 | VAL | A1201 | 134.819 | 186.856 | 193.566 | 1.00 | 6.93 | H |
| ATOM | 14471 | HG21 | VAL | A1201 | 136.220 | 186.791 | 195.595 | 1.00 | 6.93 | H |
| ATOM | 14472 | HG22 | VAL | A1201 | 135.880 | 185.270 | 195.862 | 1.00 | 6.93 | H |
| ATOM | 14473 | HG23 | VAL | A1201 | 137.393 | 185.732 | 195.715 | 1.00 | 6.93 | H |
| ATOM | 14474 | N | LEU | A1202 | 137.285 | 183.214 | 192.035 | 1.00 | 6.12 | N |
| ATOM | 14475 | CA | LEU | A1202 | 137.714 | 182.735 | 190.725 | 1.00 | 6.12 | C |
| ATOM | 14476 | C | LEU | A1202 | 136.765 | 181.668 | 190.192 | 1.00 | 6.12 | C |
| ATOM | 14477 | O | LEU | A1202 | 136.412 | 181.675 | 189.006 | 1.00 | 6.12 | O |
| ATOM | 14478 | CB | LEU | A1202 | 139.142 | 182.195 | 190.810 | 1.00 | 6.12 | C |
| ATOM | 14479 | CG | LEU | A1202 | 140.233 | 183.225 | 191.122 | 1.00 | 6.12 | C |
| ATOM | 14480 | CD1 | LEU | A1202 | 141.603 | 182.562 | 191.169 | 1.00 | 6.12 | C |
| ATOM | 14481 | CD2 | LEU | A1202 | 140.224 | 184.365 | 190.113 | 1.00 | 6.12 | C |
| ATOM | 14482 | H | LEU | A1202 | 137.840 | 183.061 | 192.675 | 1.00 | 6.12 | H |
| ATOM | 14483 | HA | LEU | A1202 | 137.705 | 183.476 | 190.099 | 1.00 | 6.12 | H |
| ATOM | 14484 | HB2 | LEU | A1202 | 139.172 | 181.523 | 191.509 | 1.00 | 6.12 | H |
| ATOM | 14485 | HB3 | LEU | A1202 | 139.365 | 181.786 | 189.959 | 1.00 | 6.12 | H |
| ATOM | 14486 | HG | LEU | A1202 | 140.062 | 183.607 | 191.996 | 1.00 | 6.12 | H |
| ATOM | 14487 | HD11 | LEU | A1202 | 142.273 | 183.235 | 191.366 | 1.00 | 6.12 | H |
| ATOM | 14488 | HD12 | LEU | A1202 | 141.601 | 181.883 | 191.862 | 1.00 | 6.12 | H |
| ATOM | 14489 | HD13 | LEU | A1202 | 141.787 | 182.155 | 190.308 | 1.00 | 6.12 | H |
| ATOM | 14490 | HD21 | LEU | A1202 | 141.026 | 184.897 | 190.231 | 1.00 | 6.12 | H |
| ATOM | 14491 | HD22 | LEU | A1202 | 140.201 | 183.994 | 189.218 | 1.00 | 6.12 | H |
| ATOM | 14492 | HD23 | LEU | A1202 | 139.439 | 184.914 | 190.264 | 1.00 | 6.12 | H |
| ATOM | 14493 | N | TYR | A1203 | 136.337 | 180.743 | 191.055 | 1.00 | 6.87 | N |
| ATOM | 14494 | CA | TYR | A1203 | 135.408 | 179.707 | 190.614 | 1.00 | 6.87 | C |
| ATOM | 14495 | C | TYR | A1203 | 134.077 | 180.310 | 190.178 | 1.00 | 6.87 | C |
| ATOM | 14496 | O | TYR | A1203 | 133.490 | 179.879 | 189.179 | 1.00 | 6.87 | O |
| ATOM | 14497 | CB | TYR | A1203 | 135.199 | 178.680 | 191.729 | 1.00 | 6.87 | C |
| ATOM | 14498 | CG | TYR | A1203 | 136.368 | 177.734 | 191.955 | 1.00 | 6.87 | C |
| ATOM | 14499 | CD1 | TYR | A1203 | 137.437 | 177.678 | 191.066 | 1.00 | 5.87 | C |
| ATOM | 14500 | CD2 | TYR | A1203 | 136.396 | 176.891 | 193.059 | 1.00 | 6.87 | C |
| ATOM | 14501 | CE1 | TYR | A1203 | 138.497 | 176.816 | 191.273 | 1.00 | 6.87 | C |
| ATOM | 14502 | CE2 | TYR | A1203 | 137.453 | 176.025 | 193.274 | 1.00 | 6.87 | C |
| ATOM | 14503 | CZ | TYR | A1203 | 138.499 | 175.991 | 192.378 | 1.00 | 6.87 | C |
| ATOM | 14504 | OH | TYR | A1203 | 139.554 | 175.132 | 192.586 | 1.00 | 6.87 | O |
| ATOM | 14505 | H | TYR | A1203 | 136.566 | 180.694 | 191.882 | 1.00 | 6.87 | H |
| ATOM | 14506 | HA | TYR | A1203 | 135.776 | 179.256 | 189.838 | 1.00 | 6.87 | H |
| ATOM | 14507 | HB2 | TYR | A1203 | 135.041 | 179.154 | 192.561 | 1.00 | 6.87 | H |
| ATOM | 14508 | HB3 | TYR | A1203 | 134.423 | 178.140 | 191.511 | 1.00 | 6.87 | H |
| ATOM | 14509 | HD1 | TYR | A1203 | 137.444 | 178.230 | 190.319 | 1.00 | 6.87 | H |
| ATOM | 14510 | HD2 | TYR | A1203 | 135.692 | 176.910 | 193.666 | 1.00 | 6.87 | H |
| ATOM | 14511 | HE1 | TYR | A1203 | 139.204 | 176.791 | 190.670 | 1.00 | 6.87 | H |
| ATOM | 14512 | HE2 | TYR | A1203 | 137.457 | 175.467 | 194.018 | 1.00 | 6.87 | H |
| ATOM | 14513 | HH | TYR | A1203 | 139.436 | 174.698 | 193.295 | 1.00 | 6.87 | H |
| ATOM | 14514 | N | TRP | A1204 | 133.582 | 181.308 | 190.914 | 1.00 | 6.27 | N |
| ATOM | 14515 | CA | TRP | A1204 | 132.326 | 181.944 | 190.525 | 1.00 | 6.27 | C |
| ATOM | 14516 | C | TRP | A1204 | 132.465 | 182.673 | 189.191 | 1.00 | 6.27 | C |
| ATOM | 14517 | O | TRP | A1204 | 131.551 | 182.638 | 188.357 | 1.00 | 6.27 | O |
| ATOM | 14518 | CB | TRP | A1204 | 131.860 | 182.901 | 191.621 | 1.00 | 6.27 | C |
| ATOM | 14519 | CG | TRP | A1204 | 131.146 | 182.205 | 192.743 | 1.00 | 6.27 | C |
| ATOM | 14520 | CD1 | TRP | A1204 | 131.610 | 182.003 | 194.011 | 1.00 | 6.27 | C |
| ATOM | 14521 | CD2 | TRP | A1204 | 129.845 | 181.607 | 192.693 | 1.00 | 6.27 | C |
| ATOM | 14522 | NE1 | TRP | A1204 | 130.677 | 181.322 | 194.754 | 1.00 | 5.27 | N |
| ATOM | 14523 | CE2 | TRP | A1204 | 129.585 | 181.066 | 193.968 | 1.00 | 6.27 | C |
| ATOM | 14524 | CE3 | TRP | A1204 | 128.875 | 181.478 | 191.695 | 1.00 | 6.27 | C |
| ATOM | 14525 | CZ2 | TRP | A1204 | 128.394 | 180.408 | 194.271 | 1.00 | 6.27 | C |
| ATOM | 14526 | CZ3 | TRP | A1204 | 127.693 | 180.825 | 191.998 | 1.00 | 6.27 | C |
| ATOM | 14527 | CH2 | TRP | A1204 | 127.464 | 180.298 | 193.275 | 1.00 | 6.27 | C |
| ATOM | 14528 | H | TRP | A1204 | 133.944 | 181.627 | 191.626 | 1.00 | 6.27 | H |
| ATOM | 14529 | HA | TRP | A1204 | 131.647 | 181.259 | 190.420 | 1.00 | 6.27 | H |
| ATOM | 14530 | HB2 | TRP | A1204 | 132.632 | 183.354 | 191.994 | 1.00 | 6.27 | H |
| ATOM | 14531 | HB3 | TRP | A1204 | 131.250 | 183.549 | 191.236 | 1.00 | 6.27 | H |
| ATOM | 14532 | HD1 | TRP | A1204 | 132.437 | 182.287 | 194.327 | 1.00 | 6.27 | H |
| ATOM | 14533 | HE1 | TRP | A1204 | 130.765 | 181.092 | 195.578 | 1.00 | 6.27 | H |
| ATOM | 14534 | HE3 | TRP | A1204 | 129.020 | 181.826 | 190.845 | 1.00 | 6.27 | H |
| ATOM | 14535 | HZ2 | TRP | A1204 | 128.239 | 180.057 | 195.118 | 1.00 | 6.27 | H |
| ATOM | 14536 | HZ3 | TRP | A1204 | 127.041 | 180.733 | 191.342 | 1.00 | 6.27 | H |
| ATOM | 14537 | HH2 | TRP | A1204 | 126.660 | 179.863 | 193.450 | 1.00 | 6.27 | H |
| ATOM | 14538 | N | ILE | A1205 | 133.601 | 183.337 | 188.968 | 1.00 | 5.39 | N |
| ATOM | 14539 | CA | ILE | A1205 | 133.829 | 183.991 | 187.681 | 1.00 | 5.39 | C |
| ATOM | 14540 | C | ILE | A1205 | 133.842 | 182.958 | 186.560 | 1.00 | 5.39 | C |
| ATOM | 14541 | O | ILE | A1205 | 133.293 | 183.185 | 185.470 | 1.00 | 5.39 | O |
| ATOM | 14542 | CB | ILE | A1205 | 135.137 | 184.803 | 187.722 | 1.00 | 5.39 | C |
| ATOM | 14543 | CG1 | ILE | A1205 | 134.982 | 186.009 | 188.652 | 1.00 | 5.39 | C |
| ATOM | 14544 | CG2 | ILE | A1205 | 135.527 | 185.253 | 186.319 | 1.00 | 5.39 | C |
| ATOM | 14545 | CD1 | ILE | A1205 | 136.292 | 186.688 | 189.004 | 1.00 | 5.39 | C |
| ATOM | 14546 | H | ILE | A1205 | 134.242 | 183.422 | 189.535 | 1.00 | 5.39 | H |
| ATOM | 14547 | HA | ILE | A1205 | 133.101 | 184.608 | 187.509 | 1.00 | 5.39 | H |
| ATOM | 14548 | HB | ILE | A1205 | 135.842 | 184.233 | 188.070 | 1.00 | 5.39 | H |
| ATOM | 14549 | HG12 | ILE | A1205 | 134.415 | 186.666 | 188.219 | 1.00 | 5.39 | H |
| ATOM | 14550 | HG13 | ILE | A1205 | 134.571 | 185.715 | 189.480 | 1.00 | 5.39 | H |
| ATOM | 14551 | HG21 | ILE | A1205 | 136.186 | 185.961 | 186.385 | 1.00 | 5.39 | H |
| ATOM | 14552 | HG22 | ILE | A1205 | 135.903 | 184.501 | 185.836 | 1.00 | 5.39 | H |
| ATOM | 14553 | HG23 | ILE | A1205 | 134.737 | 185.581 | 185.863 | 1.00 | 5.39 | H |
| ATOM | 14554 | HD11 | ILE | A1205 | 136.137 | 187.314 | 189.729 | 1.00 | 5.39 | H |
| ATOM | 14555 | HD12 | ILE | A1205 | 136.932 | 186.014 | 189.281 | 1.00 | 5.39 | H |
| ATOM | 14556 | HD13 | ILE | A1205 | 136.625 | 187.161 | 188.225 | 1.00 | 5.39 | H |
| ATOM | 14557 | N | ASN | A1206 | 134.473 | 181.808 | 186.806 | 1.00 | 5.12 | N |
| ATOM | 14558 | CA | ASN | A1206 | 134.484 | 180.747 | 185.804 | 1.00 | 5.12 | C |
| ATOM | 14559 | C | ASN | A1206 | 133.073 | 180.245 | 185.518 | 1.00 | 5.12 | C |
| ATOM | 14560 | O | ASN | A1206 | 132.730 | 179.955 | 184.365 | 1.00 | 5.12 | O |
| ATOM | 14561 | CB | ASN | A1206 | 135.381 | 179.600 | 186.267 | 1.00 | 5.12 | C |
| ATOM | 14562 | CG | ASN | A1206 | 136.850 | 179.872 | 186.013 | 1.00 | 5.12 | C |
| ATOM | 14563 | OD1 | ASN | A1206 | 137.432 | 179.358 | 185.058 | 1.00 | 5.12 | O |
| ATOM | 14564 | ND2 | ASN | A1206 | 137.459 | 180.686 | 186.867 | 1.00 | 5.12 | N |
| ATOM | 14565 | H | ASN | A1206 | 134.895 | 181.622 | 187.532 | 1.00 | 5.12 | H |
| ATOM | 14566 | HA | ASN | A1206 | 134.850 | 181.098 | 184.977 | 1.00 | 5.12 | H |
| ATOM | 14567 | HB2 | ASN | A1206 | 135.260 | 179.469 | 187.221 | 1.00 | 5.12 | H |
| ATOM | 14568 | HB3 | ASN | A1206 | 135.137 | 178.793 | 185.787 | 1.00 | 5.12 | H |
| ATOM | 14569 | HD21 | ASN | A1206 | 138.292 | 180.871 | 186.765 | 1.00 | 5.12 | H |
| ATOM | 14570 | HD22 | ASN | A1206 | 137.019 | 181.027 | 187.523 | 1.00 | 5.12 | H |
| ATOM | 14571 | N | VAL | A1207 | 132.242 | 180.123 | 186.556 | 1.00 | 6.12 | N |
| ATOM | 14572 | CA | VAL | A1207 | 130.858 | 179.699 | 186.340 | 1.00 | 6.12 | C |
| ATOM | 14573 | C | VAL | A1207 | 130.117 | 180.730 | 185.497 | 1.00 | 6.12 | C |
| ATOM | 14574 | O | VAL | A1207 | 129.292 | 180.382 | 184.642 | 1.00 | 6.12 | O |
| ATOM | 14575 | CB | VAL | A1207 | 130.132 | 179.446 | 187.682 | 1.00 | 6.12 | C |
| ATOM | 14576 | CG1 | VAL | A1207 | 130.775 | 178.418 | 188.645 | 1.00 | 6.12 | C |
| ATOM | 14577 | CG2 | VAL | A1207 | 129.141 | 180.535 | 188.171 | 1.00 | 6.12 | C |
| ATOM | 14578 | H | VAL | A1207 | 132.465 | 180.275 | 187.372 | 1.00 | 6.12 | H |
| ATOM | 14579 | HA | VAL | A1207 | 130.862 | 178.864 | 185.846 | 1.00 | 6.12 | H |
| ATOM | 14580 | HB | VAL | A1207 | 130.777 | 180.045 | 188.082 | 1.00 | 6.12 | H |
| ATOM | 14581 | HG11 | VAL | A1207 | 130.530 | 178.644 | 189.556 | 1.00 | 6.12 | H |
| ATOM | 14582 | HG12 | VAL | A1207 | 130.447 | 177.532 | 188.424 | 1.00 | 6.12 | H |
| ATOM | 14583 | HG13 | VAL | A1207 | 131.737 | 178.449 | 188.544 | 1.00 | 6.12 | H |
| ATOM | 14584 | HG21 | VAL | A1207 | 128.878 | 180.334 | 189.082 | 1.00 | 6.12 | H |
| ATOM | 14585 | HG22 | VAL | A1207 | 129.574 | 181.401 | 188.136 | 1.00 | 6.12 | H |
| ATOM | 14586 | HG23 | VAL | A1207 | 128.361 | 180.533 | 187.594 | 1.00 | 6.12 | H |
| ATOM | 14587 | N | VAL | A1208 | 130.388 | 182.015 | 185.732 | 1.00 | 5.50 | N |
| ATOM | 14588 | CA | VAL | A1208 | 129.768 | 183.063 | 184.922 | 1.00 | 5.50 | C |
| ATOM | 14589 | C | VAL | A1208 | 130.164 | 182.900 | 183.458 | 1.00 | 5.50 | C |
| ATOM | 14590 | O | VAL | A1208 | 129.328 | 183.002 | 182.549 | 1.00 | 5.50 | O |
| ATOM | 14591 | CB | VAL | A1208 | 130.154 | 184.454 | 185.459 | 1.00 | 5.50 | C |
| ATOM | 14592 | CG1 | VAL | A1208 | 129.787 | 185.537 | 184.452 | 1.00 | 5.50 | C |
| ATOM | 14593 | CG2 | VAL | A1208 | 129.480 | 184.713 | 186.797 | 1.00 | 5.50 | C |
| ATOM | 14594 | H | VAL | A1208 | 130.919 | 182.302 | 186.345 | 1.00 | 5.50 | H |
| ATOM | 14595 | HA | VAL | A1208 | 128.803 | 182.978 | 184.982 | 1.00 | 5.50 | H |
| ATOM | 14596 | HB | VAL | A1208 | 131.113 | 184.485 | 185.595 | 1.00 | 5.50 | H |
| ATOM | 14597 | HG11 | VAL | A1208 | 129.767 | 186.394 | 184.907 | 1.00 | 5.50 | H |
| ATOM | 14598 | HG12 | VAL | A1208 | 130.451 | 185.556 | 183.746 | 1.00 | 5.50 | H |
| ATOM | 14599 | HG13 | VAL | A1208 | 128.912 | 185.342 | 184.082 | 1.00 | 5.50 | H |
| ATOM | 14600 | HG21 | VAL | A1208 | 129.769 | 185.576 | 187.134 | 1.00 | 5.50 | H |
| ATOM | 14601 | HG22 | VAL | A1208 | 128.518 | 184.711 | 186.672 | 1.00 | 5.50 | H |
| ATOM | 14602 | HG23 | VAL | A1208 | 129.734 | 184.014 | 187.419 | 1.00 | 5.50 | H |
| ATOM | 14603 | N | PHE | A1209 | 131.451 | 182.650 | 183.211 | 1.00 | 5.46 | N |
| ATOM | 14604 | CA | PHE | A1209 | 131.908 | 182.439 | 181.838 | 1.00 | 5.46 | C |
| ATOM | 14605 | C | PHE | A1209 | 131.227 | 181.226 | 181.211 | 1.00 | 5.46 | C |
| ATOM | 14606 | O | PHE | A1209 | 130.847 | 181.251 | 180.032 | 1.00 | 5.46 | O |
| ATOM | 14607 | CB | PHE | A1209 | 133.427 | 182.267 | 181.811 | 1.00 | 5.46 | C |
| ATOM | 14608 | CG | PHE | A1209 | 134.184 | 183.562 | 181.722 | 1.00 | 5.46 | C |
| ATOM | 14609 | CD1 | PHE | A1209 | 134.120 | 184.343 | 180.579 | 1.00 | 5.46 | C |
| ATOM | 14610 | CD2 | PHE | A1209 | 134.965 | 183.997 | 182.780 | 1.00 | 5.46 | C |
| ATOM | 14611 | CE1 | PHE | A1209 | 134.817 | 185.533 | 180.495 | 1.00 | 5.46 | C |
| ATOM | 14612 | CE2 | PHE | A1209 | 135.664 | 185.187 | 182.701 | 1.00 | 5.46 | C |
| ATOM | 14613 | CZ | PHE | A1209 | 135.590 | 185.955 | 181.558 | 1.00 | 5.46 | C |
| ATOM | 14614 | H | PHE | A1209 | 132.068 | 182.599 | 183.808 | 1.00 | 5.46 | H |
| ATOM | 14615 | HA | PHE | A1209 | 131.682 | 183.217 | 181.307 | 1.00 | 5.46 | H |
| ATOM | 14616 | HB2 | PHE | A1209 | 133.706 | 181.817 | 182.624 | 1.00 | 5.46 | H |
| ATOM | 14617 | HB3 | PHE | A1209 | 133.667 | 181.731 | 181.040 | 1.00 | 5.46 | H |
| ATOM | 14618 | HD1 | PHE | A1209 | 133.600 | 184.063 | 179.860 | 1.00 | 5.46 | H |
| ATOM | 14619 | HD2 | PHE | A1209 | 135.018 | 183.483 | 183.553 | 1.00 | 5.46 | H |
| ATOM | 14620 | HE1 | PHE | A1209 | 134.766 | 186.049 | 179.723 | 1.00 | 5.46 | H |
| ATOM | 14621 | HE2 | PHE | A1209 | 136.184 | 185.469 | 183.419 | 1.00 | 5.46 | H |
| ATOM | 14622 | HZ | PHE | A1209 | 136.060 | 186.756 | 181.503 | 1.00 | 5.46 | H |
| ATOM | 14623 | N | ILE | A1210 | 131.080 | 180.147 | 181.983 | 1.00 | 6.24 | N |
| ATOM | 14624 | CA | ILE | A1210 | 130.420 | 178.949 | 181.473 | 1.00 | 6.24 | C |
| ATOM | 14625 | C | ILE | A1210 | 128.979 | 179.262 | 181.094 | 1.00 | 6.24 | C |
| ATOM | 14626 | O | ILE | A1210 | 128.478 | 178.807 | 180.059 | 1.00 | 6.24 | O |
| ATOM | 14627 | CB | ILE | A1210 | 130.498 | 177.814 | 182.513 | 1.00 | 6.24 | C |
| ATOM | 14628 | CG1 | ILE | A1210 | 131.882 | 177.163 | 182.483 | 1.00 | 6.24 | C |
| ATOM | 14629 | CG2 | ILE | A1210 | 129.419 | 176.769 | 182.253 | 1.00 | 6.24 | C |
| ATOM | 14630 | CD1 | ILE | A1210 | 132.196 | 176.339 | 183.712 | 1.00 | 6.24 | C |
| ATOM | 14631 | H | ILE | A1210 | 131.352 | 180.086 | 182.797 | 1.00 | 6.24 | H |
| ATOM | 14632 | HA | ILE | A1210 | 130.881 | 178.652 | 180.673 | 1.00 | 6.24 | H |
| ATOM | 14633 | HB | ILE | A1210 | 130.354 | 178.193 | 183.394 | 1.00 | 6.24 | H |
| ATOM | 14634 | HG12 | ILE | A1210 | 131.935 | 176.578 | 181.711 | 1.00 | 6.24 | H |
| ATOM | 14635 | HG13 | ILE | A1210 | 132.553 | 177.860 | 182.414 | 1.00 | 6.24 | H |
| ATOM | 14636 | HG21 | ILE | A1210 | 129.649 | 175.956 | 182.729 | 1.00 | 6.24 | H |
| ATOM | 14637 | HG22 | ILE | A1210 | 128.566 | 177.103 | 182.572 | 1.00 | 6.24 | H |
| ATOM | 14638 | HG23 | ILE | A1210 | 129.374 | 176.589 | 181.301 | 1.00 | 6.24 | H |
| ATOM | 14639 | HD11 | ILE | A1210 | 133.154 | 176.193 | 183.759 | 1.00 | 6.24 | H |
| ATOM | 14640 | HD12 | ILE | A1210 | 131.895 | 176.819 | 184.499 | 1.00 | 6.24 | H |
| ATOM | 14641 | HD13 | ILE | A1210 | 131.734 | 175.488 | 183.648 | 1.00 | 6.24 | H |
| ATOM | 14642 | N | ILE | A1211 | 128.286 | 180.033 | 181.935 | 1.00 | 6.76 | N |
| ATOM | 14643 | CA | ILE | A1211 | 126.910 | 180.415 | 181.631 | 1.00 | 6.76 | C |
| ATOM | 14644 | C | ILE | A1211 | 126.859 | 181.225 | 180.342 | 1.00 | 6.76 | C |
| ATOM | 14645 | O | ILE | A1211 | 125.964 | 181.038 | 179.507 | 1.00 | 6.76 | O |
| ATOM | 14646 | CB | ILE | A1211 | 126.298 | 181.194 | 182.810 | 1.00 | 6.76 | C |
| ATOM | 14647 | CG1 | ILE | A1211 | 126.085 | 180.263 | 184.006 | 1.00 | 6.76 | C |
| ATOM | 14648 | CG2 | ILE | A1211 | 124.984 | 181.846 | 182.391 | 1.00 | 6.76 | C |
| ATOM | 14649 | CD1 | ILE | A1211 | 125.759 | 180.986 | 185.296 | 1.00 | 6.76 | C |
| ATOM | 14650 | H | ILE | A1211 | 128.587 | 180.343 | 182.678 | 1.00 | 6.76 | H |
| ATOM | 14651 | HA | ILE | A1211 | 126.382 | 179.612 | 181.499 | 1.00 | 6.76 | H |
| ATOM | 14652 | HB | ILE | A1211 | 126.918 | 181.893 | 183.071 | 1.00 | 6.76 | H |
| ATOM | 14653 | HG12 | ILE | A1211 | 125.348 | 179.665 | 183.807 | 1.00 | 6.76 | H |
| ATOM | 14654 | HG13 | ILE | A1211 | 126.895 | 179.750 | 184.151 | 1.00 | 6.76 | H |
| ATOM | 14655 | HG21 | ILE | A1211 | 124.486 | 182.098 | 183.183 | 1.00 | 6.76 | H |
| ATOM | 14656 | HG22 | ILE | A1211 | 125.174 | 182.635 | 181.860 | 1.00 | 6.76 | H |
| ATOM | 14657 | HG23 | ILE | A1211 | 124.470 | 181.210 | 181.869 | 1.00 | 6.76 | H |
| ATOM | 14658 | HD11 | ILE | A1211 | 125.836 | 180.363 | 186.035 | 1.00 | 6.76 | H |
| ATOM | 14659 | HD12 | ILE | A1211 | 126.385 | 181.718 | 185.414 | 1.00 | 6.76 | H |
| ATOM | 14660 | HD13 | ILE | A1211 | 124.854 | 181.331 | 185.247 | 1.00 | 6.76 | H |
| ATOM | 14661 | N | LEU | A1212 | 127.807 | 182.148 | 180.167 | 1.00 | 5.76 | N |
| ATOM | 14662 | CA | LEU | A1212 | 127.823 | 182.959 | 178.952 | 1.00 | 5.76 | C |
| ATOM | 14663 | C | LEU | A1212 | 128.017 | 182.092 | 177.711 | 1.00 | 5.76 | C |
| ATOM | 14664 | O | LEU | A1212 | 127.337 | 182.283 | 176.693 | 1.00 | 5.76 | O |
| ATOM | 14665 | CB | LEU | A1212 | 128.923 | 184.016 | 179.044 | 1.00 | 5.76 | C |
| ATOM | 14666 | CG | LEU | A1212 | 128.609 | 185.236 | 179.913 | 1.00 | 5.76 | C |
| ATOM | 14667 | CD1 | LEU | A1212 | 129.853 | 186.091 | 180.097 | 1.00 | 5.76 | C |
| ATOM | 14668 | CD2 | LEU | A1212 | 127.478 | 186.057 | 179.310 | 1.00 | 5.76 | C |
| ATOM | 14669 | H | LEU | A1212 | 128.439 | 182.320 | 180.724 | 1.00 | 5.76 | H |
| ATOM | 14670 | HA | LEU | A1212 | 126.971 | 183.413 | 178.866 | 1.00 | 5.76 | H |
| ATOM | 14671 | HB2 | LEU | A1212 | 129.718 | 183.598 | 179.409 | 1.00 | 5.76 | H |
| ATOM | 14672 | HB3 | LEU | A1212 | 129.111 | 184.339 | 178.149 | 1.00 | 5.76 | H |
| ATOM | 14673 | HG | LEU | A1212 | 128.323 | 184.933 | 180.789 | 1.00 | 5.76 | H |
| ATOM | 14674 | HD11 | LEU | A1212 | 129.645 | 186.826 | 180.695 | 1.00 | 5.76 | H |
| ATOM | 14675 | HD12 | LEU | A1212 | 130.558 | 185.544 | 180.478 | 1.00 | 5.76 | H |
| ATOM | 14676 | HD13 | LEU | A1212 | 130.131 | 186.434 | 179.234 | 1.00 | 5.76 | H |
| ATOM | 14677 | HD21 | LEU | A1212 | 127.417 | 186.903 | 179.781 | 1.00 | 5.76 | H |
| ATOM | 14678 | HD22 | LEU | A1212 | 127.667 | 186.213 | 178.372 | 1.00 | 5.76 | H |
| ATOM | 14679 | HD23 | LEU | A1212 | 126.646 | 185.567 | 179.403 | 1.00 | 5.76 | H |
| ATOM | 14680 | N | PHE | A1213 | 128.945 | 181.136 | 177.774 | 1.00 | 6.22 | N |
| ATOM | 14681 | CA | PHE | A1213 | 129.167 | 180.257 | 176.627 | 1.00 | 6.22 | C |
| ATOM | 14682 | C | PHE | A1213 | 127.944 | 179.387 | 176.353 | 1.00 | 6.22 | C |
| ATOM | 14683 | O | PHE | A1213 | 127.591 | 179.146 | 175.190 | 1.00 | 6.22 | O |
| ATOM | 14684 | CB | PHE | A1213 | 130.410 | 179.397 | 176.859 | 1.00 | 6.22 | C |
| ATOM | 14685 | CG | PHE | A1213 | 131.690 | 180.187 | 176.923 | 1.00 | 6.22 | C |
| ATOM | 14686 | CD1 | PHE | A1213 | 131.949 | 181.191 | 176.003 | 1.00 | 6.22 | C |
| ATOM | 14687 | CD2 | PHE | A1213 | 132.631 | 179.930 | 177.906 | 1.00 | 6.22 | C |
| ATOM | 14688 | CE1 | PHE | A1213 | 133.122 | 181.919 | 176.060 | 1.00 | 6.22 | C |
| ATOM | 14689 | CE2 | PHE | A1213 | 133.806 | 180.656 | 177.968 | 1.00 | 6.22 | C |
| ATOM | 14690 | CZ | PHE | A1213 | 134.052 | 181.651 | 177.044 | 1.00 | 6.22 | C |
| ATOM | 14691 | H | PHE | A1213 | 129.449 | 180.978 | 178.453 | 1.00 | 6.22 | H |
| ATOM | 14692 | HA | PHE | A1213 | 129.316 | 180.802 | 175.839 | 1.00 | 6.22 | H |
| ATOM | 14693 | HB2 | PHE | A1213 | 130.311 | 178.925 | 177.700 | 1.00 | 6.22 | H |
| ATOM | 14694 | HB3 | PHE | A1213 | 130.490 | 178.761 | 176.132 | 1.00 | 6.22 | H |
| ATOM | 14695 | HD1 | PHE | A1213 | 131.327 | 181.376 | 175.337 | 1.00 | 6.22 | H |
| ATOM | 14696 | HD2 | PHE | A1213 | 132.470 | 179.260 | 178.531 | 1.00 | 6.22 | H |
| ATOM | 14697 | HE1 | PHE | A1213 | 133.285 | 182.589 | 175.436 | 1.00 | 6.22 | H |
| ATOM | 14698 | HE2 | PHE | A1213 | 134.431 | 180.474 | 178.632 | 1.00 | 6.22 | H |
| ATOM | 14699 | HZ | PHE | A1213 | 134.842 | 182.140 | 177.085 | 1.00 | 6.22 | H |
| ATOM | 14700 | N | THR | A1214 | 127.284 | 178.906 | 177.409 | 1.00 | 8.09 | N |
| ATOM | 14701 | CA | THR | A1214 | 126.057 | 178.139 | 177.222 | 1.00 | 8.09 | C |
| ATOM | 14702 | C | THR | A1214 | 124.989 | 178.981 | 176.535 | 1.00 | 8.09 | C |
| ATOM | 14703 | O | THR | A1214 | 124.275 | 178.494 | 175.650 | 1.00 | 8.09 | O |
| ATOM | 14704 | CB | THR | A1214 | 125.545 | 177.628 | 178.569 | 1.00 | 8.09 | C |
| ATOM | 14705 | OG1 | THR | A1214 | 126.548 | 176.814 | 179.190 | 1.00 | 8.09 | O |
| ATOM | 14706 | CG2 | THR | A1214 | 124.273 | 176.809 | 178.386 | 1.00 | 8.09 | C |
| ATOM | 14707 | H | THR | A1214 | 127.522 | 179.009 | 178.229 | 1.00 | 8.09 | H |
| ATOM | 14708 | HA | THR | A1214 | 126.244 | 177.372 | 176.659 | 1.00 | 8.09 | H |
| ATOM | 14709 | HB | THR | A1214 | 125.342 | 178.383 | 179.143 | 1.00 | 8.09 | H |
| ATOM | 14710 | HG1 | THR | A1214 | 126.270 | 176.529 | 179.929 | 1.00 | 8.09 | H |
| ATOM | 14711 | HG21 | THR | A1214 | 124.071 | 176.327 | 179.203 | 1.00 | 8.09 | H |
| ATOM | 14712 | HG22 | THR | A1214 | 123.527 | 177.391 | 178.173 | 1.00 | 8.09 | H |
| ATOM | 14713 | HG23 | THR | A1214 | 124.391 | 176.170 | 177.666 | 1.00 | 8.09 | H |
| ATOM | 14714 | N | GLY | A1215 | 124.859 | 180.245 | 176.938 | 1.00 | 9.16 | N |
| ATOM | 14715 | CA | GLY | A1215 | 123.908 | 181.126 | 176.281 | 1.00 | 9.16 | C |
| ATOM | 14716 | C | GLY | A1215 | 124.238 | 181.343 | 174.817 | 1.00 | 9.16 | C |
| ATOM | 14717 | O | GLY | A1215 | 123.344 | 181.378 | 173.966 | 1.00 | 9.16 | O |
| ATOM | 14718 | H | GLY | A1215 | 125.303 | 180.608 | 177.579 | 1.00 | 9.16 | H |
| ATOM | 14719 | HA2 | GLY | A1215 | 123.018 | 180.744 | 176.342 | 1.00 | 9.16 | H |
| ATOM | 14720 | HA3 | GLY | A1215 | 123.904 | 181.987 | 176.726 | 1.00 | 9.16 | H |
| ATOM | 14721 | N | GLU | A1216 | 125.526 | 181.502 | 174.505 | 1.00 | 10.96 | N |
| ATOM | 14722 | CA | GLU | A1216 | 125.936 | 181.624 | 173.108 | 1.00 | 10.96 | C |
| ATOM | 14723 | C | GLU | A1216 | 125.514 | 180.391 | 172.316 | 1.00 | 10.96 | C |
| ATOM | 14724 | O | GLU | A1216 | 124.975 | 180.498 | 171.205 | 1.00 | 10.96 | O |
| ATOM | 14725 | CB | GLU | A1216 | 127.451 | 181.829 | 173.028 | 1.00 | 10.96 | C |
| ATOM | 14726 | CG | GLU | A1216 | 128.046 | 181.767 | 171.613 | 1.00 | 10.96 | C |
| ATOM | 14727 | CD | GLU | A1216 | 129.404 | 181.084 | 171.561 | 1.00 | 10.96 | C |
| ATOM | 14728 | OE1 | GLU | A1216 | 129.829 | 180.496 | 172.578 | 1.00 | 10.96 | O |
| ATOM | 14729 | OE2 | GLU | A1216 | 130.047 | 181.132 | 170.491 | 1.00 | 10.96 | O |
| ATOM | 14730 | H | GLU | A1216 | 126.170 | 181.542 | 175.073 | 1.00 | 10.96 | H |
| ATOM | 14731 | HA | GLU | A1216 | 125.504 | 182.399 | 172.715 | 1.00 | 10.96 | H |
| ATOM | 14732 | HB2 | GLU | A1216 | 127.663 | 182.702 | 173.394 | 1.00 | 10.96 | H |
| ATOM | 14733 | HB3 | GLU | A1216 | 127.875 | 181.143 | 173.564 | 1.00 | 10.96 | H |
| ATOM | 14734 | HG2 | GLU | A1216 | 127.454 | 181.290 | 171.014 | 1.00 | 10.96 | H |
| ATOM | 14735 | HG3 | GLU | A1216 | 128.167 | 182.674 | 171.294 | 1.00 | 10.96 | H |
| ATOM | 14736 | N | CYS | A1217 | 125.760 | 179.205 | 172.877 | 1.00 | 12.05 | N |
| ATOM | 14737 | CA | CYS | A1217 | 125.382 | 177.973 | 172.191 | 1.00 | 12.05 | C |
| ATOM | 14738 | C | CYS | A1217 | 123.875 | 177.901 | 171.979 | 1.00 | 12.05 | C |
| ATOM | 14739 | O | CYS | A1217 | 123.409 | 177.501 | 170.905 | 1.00 | 12.05 | O |
| ATOM | 14740 | CB | CYS | A1217 | 125.868 | 176.760 | 172.984 | 1.00 | 12.05 | C |
| ATOM | 14741 | SG | CYS | A1217 | 125.818 | 175.203 | 172.065 | 1.00 | 12.05 | S |
| ATOM | 14742 | H | CYS | A1217 | 126.137 | 179.089 | 173.641 | 1.00 | 12.05 | H |
| ATOM | 14743 | HA | CYS | A1217 | 125.809 | 177.953 | 171.320 | 1.00 | 12.05 | H |
| ATOM | 14744 | HB2 | CYS | A1217 | 126.787 | 176.914 | 173.255 | 1.00 | 12.05 | H |
| ATOM | 14745 | HB3 | CYS | A1217 | 125.308 | 176.658 | 173.769 | 1.00 | 12.05 | H |
| ATOM | 4746 | HG | CYS | A1217 | 126.206 | 174.317 | 172.774 | 1.00 | 12.05 | H |
| ATOM | 14747 | N | VAL | A1218 | 123.096 | 178.276 | 172.996 | 1.00 | 13.11 | N |
| ATOM | 14748 | CA | VAL | A1218 | 121.641 | 178.231 | 172.872 | 1.00 | 13.11 | C |
| ATOM | 14749 | C | VAL | A1218 | 121.170 | 179.186 | 171.783 | 1.00 | 13.11 | C |
| ATOM | 14750 | O | VAL | A1218 | 120.295 | 178.848 | 170.975 | 1.00 | 13.11 | O |
| ATOM | 14751 | CB | VAL | A1218 | 120.979 | 178.546 | 174.227 | 1.00 | 13.11 | C |
| ATOM | 14752 | CG1 | VAL | A1218 | 119.471 | 178.670 | 174.069 | 1.00 | 13.11 | C |
| ATOM | 14753 | CG2 | VAL | A1218 | 121.317 | 177.469 | 175.246 | 1.00 | 13.11 | C |
| ATOM | 14754 | H | VAL | A1218 | 123.382 | 178.556 | 173.757 | 1.00 | 13.11 | H |
| ATOM | 14755 | HA | VAL | A1218 | 121.376 | 177.334 | 172.615 | 1.00 | 13.11 | H |
| ATOM | 14756 | HB | VAL | A1218 | 121.319 | 179.392 | 174.558 | 1.00 | 13.11 | H |
| ATOM | 14757 | HG11 | VAL | A1218 | 119.059 | 178.609 | 174.946 | 1.00 | 13.11 | H |
| ATOM | 14758 | HG12 | VAL | A1218 | 119.262 | 179.528 | 173.666 | 1.00 | 13.11 | H |
| ATOM | 14759 | HG13 | VAL | A1218 | 119.152 | 177.949 | 173.505 | 1.00 | 13.11 | H |
| ATOM | 14760 | HG21 | VAL | A1218 | 121.057 | 177.777 | 176.128 | 1.00 | 13.11 | H |
| ATOM | 14761 | HG22 | VAL | A1218 | 120.830 | 176.661 | 175.021 | 1.00 | 13.11 | H |
| ATOM | 14762 | HG23 | VAL | A1218 | 122.270 | 177.296 | 175.224 | 1.00 | 13.11 | H |
| ATOM | 14763 | N | LEU | A1219 | 121.733 | 180.396 | 171.749 | 1.00 | 15.91 | N |
| ATOM | 14764 | CA | LEU | A1219 | 121.347 | 181.356 | 170.721 | 1.00 | 15.91 | C |
| ATOM | 14765 | C | LEU | A1219 | 121.670 | 180.828 | 169.329 | 1.00 | 15.91 | C |
| ATOM | 14766 | O | LEU | A1219 | 120.849 | 180.934 | 168.408 | 1.00 | 15.91 | O |
| ATOM | 14767 | CB | LEU | A1219 | 122.053 | 182.693 | 170.959 | 1.00 | 15.91 | C |
| ATOM | 14768 | CG | LEU | A1219 | 121.456 | 183.655 | 171.994 | 1.00 | 15.91 | C |
| ATOM | 14769 | CD1 | LEU | A1219 | 122.156 | 185.005 | 171.920 | 1.00 | 15.91 | C |
| ATOM | 14770 | CD2 | LEU | A1219 | 119.954 | 183.831 | 171.817 | 1.00 | 15.91 | C |
| ATOM | 14771 | H | LEU | A1219 | 122.329 | 180.679 | 172.301 | 1.00 | 15.91 | H |
| ATOM | 14772 | HA | LEU | A1219 | 120.389 | 181.496 | 170.764 | 1.00 | 15.91 | H |
| ATOM | 14773 | HB2 | LEU | A1219 | 122.961 | 182.503 | 171.242 | 1.00 | 15.91 | H |
| ATOM | 14774 | HB3 | LEU | A1219 | 122.079 | 183.170 | 170.115 | 1.00 | 15.91 | H |
| ATOM | 14775 | HG | LEU | A1219 | 121.609 | 183.292 | 172.881 | 1.00 | 15.91 | H |
| ATOM | 14776 | HD11 | LEU | A1219 | 121.810 | 185.578 | 172.622 | 1.00 | 15.91 | H |
| ATOM | 14777 | HD12 | LEU | A1219 | 123.110 | 184.874 | 172.040 | 1.00 | 15.91 | H |
| ATOM | 14778 | HD13 | LEU | A1219 | 121.983 | 185.403 | 171.053 | 1.00 | 15.91 | H |
| ATOM | 14779 | HD21 | LEU | A1219 | 119.666 | 184.605 | 172.326 | 1.00 | 15.91 | H |
| ATOM | 14780 | HD22 | LEU | A1219 | 119.760 | 183.965 | 170.876 | 1.00 | 15.91 | H |
| ATOM | 14781 | HD23 | LEU | A1219 | 119.501 | 183.037 | 172.139 | 1.00 | 15.91 | H |
| ATOM | 14782 | N | LYS | A1220 | 122.863 | 180.252 | 169.154 | 1.00 | 15.12 | N |
| ATOM | 14783 | CA | LYS | A1220 | 123.227 | 179.732 | 167.840 | 1.00 | 15.12 | C |
| ATOM | 14784 | C | LYS | A1220 | 122.334 | 178.566 | 167.433 | 1.00 | 15.12 | C |
| ATOM | 14785 | O | LYS | A1220 | 121.964 | 178.453 | 166.258 | 1.00 | 15.12 | O |
| ATOM | 14786 | CB | LYS | A1220 | 124.697 | 179.314 | 167.821 | 1.00 | 15.12 | C |
| ATOM | 14787 | CG | LYS | A1220 | 125.665 | 180.487 | 167.891 | 1.00 | 15.12 | C |
| ATOM | 14788 | CD | LYS | A1220 | 127.107 | 180.022 | 168.002 | 1.00 | 15.12 | C |
| ATOM | 14789 | CE | LYS | A1220 | 127.593 | 179.402 | 166.702 | 1.00 | 15.12 | C |
| ATOM | 14790 | NZ | LYS | A1220 | 129.047 | 179.090 | 166.743 | 1.00 | 15.12 | N |
| ATOM | 14791 | H | LYS | A1220 | 123.461 | 180.151 | 169.764 | 1.00 | 15.12 | H |
| ATOM | 14792 | HA | LYS | A1220 | 123.111 | 180.436 | 167.183 | 1.00 | 15.12 | H |
| ATOM | 14793 | HB2 | LYS | A1220 | 124.870 | 178.740 | 168.584 | 1.00 | 15.12 | H |
| ATOM | 14794 | HB3 | LYS | A1220 | 124.873 | 178.832 | 166.999 | 1.00 | 15.12 | H |
| ATOM | 14795 | HG2 | LYS | A1220 | 125.579 | 181.020 | 167.085 | 1.00 | 15.12 | H |
| ATOM | 14796 | HG3 | LYS | A1220 | 125.459 | 181.026 | 168.671 | 1.00 | 15.12 | H |
| ATOM | 14797 | HD2 | LYS | A1220 | 127.674 | 180.781 | 168.208 | 1.00 | 15.12 | H |
| ATOM | 14798 | HD3 | LYS | A1220 | 127.174 | 179.353 | 168.701 | 1.00 | 15.12 | H |
| ATOM | 14799 | HE2 | LYS | A1220 | 127.110 | 178.578 | 166.538 | 1.00 | 15.12 | H |
| ATOM | 14800 | HE3 | LYS | A1220 | 127.440 | 180.027 | 165.977 | 1.00 | 15.12 | H |
| ATOM | 14801 | HZ1 | LYS | A1220 | 129.292 | 178.683 | 165.990 | 1.00 | 15.12 | H |
| ATOM | 14802 | HZ2 | LYS | A1220 | 129.517 | 179.840 | 166.833 | 1.00 | 15.12 | H |
| ATOM | 14803 | HZ3 | LYS | A1220 | 129.223 | 178.554 | 167.432 | 1.00 | 15.12 | H |
| ATOM | 14804 | N | LEU | A1221 | 121.979 | 177.693 | 168.379 | 1.00 | 15.41 | N |
| ATOM | 14805 | CA | LEU | A1221 | 121.044 | 176.615 | 168.070 | 1.00 | 15.41 | C |
| ATOM | 14806 | C | LEU | A1221 | 119.698 | 177.174 | 167.628 | 1.00 | 15.41 | C |
| ATOM | 14807 | O | LEU | A1221 | 119.147 | 176.749 | 166.603 | 1.00 | 15.41 | O |
| ATOM | 14808 | CB | LEU | A1221 | 120.867 | 175.709 | 169.290 | 1.00 | 15.41 | C |
| ATOM | 14809 | CG | LEU | A1221 | 121.749 | 174.462 | 169.390 | 1.00 | 15.41 | C |
| ATOM | 14810 | CD1 | LEU | A1221 | 121.500 | 173.750 | 170.710 | 1.00 | 15.41 | C |
| ATOM | 14811 | CD2 | LEU | A1221 | 121.494 | 173.517 | 168.228 | 1.00 | 15.41 | C |
| ATOM | 14812 | H | LEU | A1221 | 122.258 | 177.705 | 169.192 | 1.00 | 15.41 | H |
| ATOM | 14813 | HA | LEU | A1221 | 121.403 | 176.086 | 167.342 | 1.00 | 15.41 | H |
| ATOM | 14814 | HB2 | LEU | A1221 | 121.031 | 176.239 | 170.086 | 1.00 | 15.41 | H |
| ATOM | 14815 | HB3 | LEU | A1221 | 119.946 | 175.403 | 169.299 | 1.00 | 15.41 | H |
| ATOM | 14816 | HG | LEU | A1221 | 122.681 | 174.729 | 169.363 | 1.00 | 15.41 | H |
| ATOM | 14817 | HD11 | LEU | A1221 | 122.027 | 172.936 | 170.734 | 1.00 | 15.41 | H |
| ATOM | 14818 | HD12 | LEU | A1221 | 121.761 | 174.335 | 171.439 | 1.00 | 15.41 | H |
| ATOM | 14819 | HD13 | LEU | A1221 | 120.556 | 173.535 | 170.780 | 1.00 | 15.41 | H |
| ATOM | 14820 | HD21 | LEU | A1221 | 121.939 | 172.673 | 168.405 | 1.00 | 15.41 | H |
| ATOM | 14821 | HD22 | LEU | A1221 | 120.538 | 173.374 | 168.144 | 1.00 | 15.41 | H |
| ATOM | 14822 | HD23 | LEU | A1221 | 121.843 | 173.910 | 167.413 | 1.00 | 15.41 | H |
| ATOM | 14823 | N | ILE | A1222 | 119.169 | 178.147 | 168.375 | 1.00 | 19.75 | N |
| ATOM | 14824 | CA | ILE | A1222 | 117.889 | 178.750 | 168.017 | 1.00 | 19.75 | C |
| ATOM | 14825 | C | ILE | A1222 | 117.957 | 179.321 | 166.610 | 1.00 | 19.75 | C |
| ATOM | 14826 | O | ILE | A1222 | 117.017 | 179.184 | 165.818 | 1.00 | 19.75 | O |
| ATOM | 14827 | CB | ILE | A1222 | 117.506 | 179.836 | 169.040 | 1.00 | 19.75 | C |
| ATOM | 14828 | CG1 | ILE | A1222 | 116.986 | 179.201 | 170.329 | 1.00 | 19.75 | C |
| ATOM | 14829 | CG2 | ILE | A1222 | 116.460 | 180.782 | 168.456 | 1.00 | 19.75 | C |
| ATOM | 14830 | CD1 | ILE | A1222 | 117.263 | 180.034 | 171.559 | 1.00 | 19.75 | C |
| ATOM | 14831 | H | ILE | A1222 | 119.530 | 178.472 | 169.084 | 1.00 | 19.75 | H |
| ATOM | 14832 | HA | ILE | A1222 | 117.201 | 178.067 | 168.032 | 1.00 | 19.75 | H |
| ATOM | 14833 | HB | ILE | A1222 | 118.300 | 180.351 | 169.251 | 1.00 | 19.75 | H |
| ATOM | 14834 | HG12 | ILE | A1222 | 116.026 | 179.084 | 170.258 | 1.00 | 19.75 | H |
| ATOM | 14835 | HG13 | ILE | A1222 | 117.417 | 178.341 | 170.450 | 1.00 | 19.75 | H |
| ATOM | 14836 | HG21 | ILE | A1222 | 116.040 | 181.270 | 169.182 | 1.00 | 19.75 | H |
| ATOM | 14837 | HG22 | ILE | A1222 | 116.894 | 181.406 | 167.852 | 1.00 | 19.75 | H |
| ATOM | 14838 | HG23 | ILE | A1222 | 115.793 | 180.264 | 167.980 | 1.00 | 19.75 | H |
| ATOM | 14839 | HD11 | ILE | A1222 | 116.912 | 179.574 | 172.338 | 1.00 | 19.75 | H |
| ATOM | 14840 | HD12 | ILE | A1222 | 118.222 | 180.155 | 171.649 | 1.00 | 19.75 | H |
| ATOM | 14841 | HD13 | ILE | A1222 | 116.830 | 180.896 | 171.459 | 1.00 | 19.75 | H |
| ATOM | 14842 | N | SER | A1223 | 119.068 | 179.979 | 166.282 | 1.00 | 21.08 | N |
| ATOM | 14843 | CA | SER | A1223 | 119.190 | 180.609 | 164.973 | 1.00 | 21.08 | C |
| ATOM | 14844 | C | SER | A1223 | 119.260 | 179.572 | 163.857 | 1.00 | 21.08 | C |
| ATOM | 14845 | O | SER | A1223 | 118.573 | 179.700 | 162.837 | 1.00 | 21.08 | O |
| ATOM | 14846 | CB | SER | A1223 | 120.422 | 181.513 | 164.944 | 1.00 | 21.08 | C |
| ATOM | 14847 | OG | SER | A1223 | 120.075 | 182.848 | 165.264 | 1.00 | 21.08 | O |
| ATOM | 14848 | H | SER | A1223 | 119.753 | 180.074 | 166.792 | 1.00 | 21.08 | H |
| ATOM | 14849 | HA | SER | A1223 | 118.410 | 181.162 | 164.816 | 1.00 | 21.08 | H |
| ATOM | 14850 | HB2 | SER | A1223 | 121.069 | 181.191 | 165.591 | 1.00 | 21.08 | H |
| ATOM | 14851 | HB3 | SER | A1223 | 120.806 | 181.494 | 164.053 | 1.00 | 21.08 | H |
| ATOM | 14852 | HG | SER | A1223 | 120.768 | 183.321 | 165.317 | 1.00 | 21.08 | H |
| ATOM | 14853 | N | LEU | A1224 | 120.080 | 178.534 | 164.031 | 1.00 | 20.02 | N |
| ATOM | 14854 | CA | LEU | A1224 | 120.460 | 177.701 | 162.896 | 1.00 | 20.02 | C |
| ATOM | 14855 | C | LEU | A1224 | 119.635 | 176.427 | 162.744 | 1.00 | 20.02 | C |
| ATOM | 14856 | O | LEU | A1224 | 119.672 | 175.827 | 161.666 | 1.00 | 20.02 | O |
| ATOM | 14857 | CB | LEU | A1224 | 121.947 | 177.346 | 162.986 | 1.00 | 20.02 | C |
| ATOM | 14858 | CG | LEU | A1224 | 122.858 | 178.579 | 163.035 | 1.00 | 20.02 | C |
| ATOM | 14859 | CD1 | LEU | A1224 | 124.236 | 178.225 | 163.552 | 1.00 | 20.02 | C |
| ATOM | 14860 | CD2 | LEU | A1224 | 122.951 | 179.244 | 161.669 | 1.00 | 20.02 | C |
| ATOM | 14861 | H | LEU | A1224 | 120.425 | 178.297 | 164.782 | 1.00 | 20.02 | H |
| ATOM | 14862 | HA | LEU | A1224 | 120.334 | 178.217 | 162.085 | 1.00 | 20.02 | H |
| ATOM | 14863 | HB2 | LEU | A1224 | 122.103 | 176.828 | 163.791 | 1.00 | 20.02 | H |
| ATOM | 14864 | HB3 | LEU | A1224 | 122.193 | 176.827 | 162.204 | 1.00 | 20.02 | H |
| ATOM | 14865 | HG | LEU | A1224 | 122.478 | 179.224 | 163.651 | 1.00 | 20.02 | H |
| ATOM | 14866 | HD11 | LEU | A1224 | 124.791 | 179.021 | 163.538 | 1.00 | 20.02 | H |
| ATOM | 14867 | HD12 | LEU | A1224 | 124.148 | 177.900 | 164.460 | 1.00 | 20.02 | H |
| ATOM | 14868 | HD13 | LEU | A1224 | 124.625 | 177.539 | 162.986 | 1.00 | 20.02 | H |
| ATOM | 14869 | HD21 | LEU | A1224 | 123.292 | 180.145 | 161.778 | 1.00 | 20.02 | H |
| ATOM | 14870 | HD22 | LEU | A1224 | 123.549 | 178.732 | 161.102 | 1.00 | 20.02 | H |
| ATOM | 14871 | HD23 | LEU | A1224 | 122.068 | 179.275 | 161.274 | 1.00 | 20.02 | H |
| ATOM | 14872 | N | ARG | A1225 | 118.897 | 175.990 | 163.769 | 1.00 | 23.58 | N |
| ATOM | 14873 | CA | ARG | A1225 | 117.881 | 174.941 | 163.601 | 1.00 | 23.58 | C |
| ATOM | 14874 | C | ARG | A1225 | 118.377 | 173.761 | 162.762 | 1.00 | 23.58 | C |
| ATOM | 14875 | O | ARG | A1225 | 117.921 | 173.576 | 161.634 | 1.00 | 23.58 | O |
| ATOM | 14876 | CB | ARG | A1225 | 116.613 | 175.505 | 162.956 | 1.00 | 23.58 | C |
| ATOM | 14877 | CG | ARG | A1225 | 116.032 | 176.739 | 163.619 | 1.00 | 23.58 | C |
| ATOM | 14878 | CD | ARG | A1225 | 115.429 | 177.653 | 162.567 | 1.00 | 23.58 | C |
| ATOM | 14879 | NE | ARG | A1225 | 116.429 | 178.057 | 161.579 | 1.00 | 23.58 | N |
| ATOM | 14880 | CZ | ARG | A1225 | 116.165 | 178.375 | 160.315 | 1.00 | 23.58 | C |
| ATOM | 14881 | NH1 | ARG | A1225 | 114.924 | 178.325 | 159.851 | 1.00 | 23.58 | N |
| ATOM | 14882 | NH2 | ARG | A1225 | 117.154 | 178.732 | 159.507 | 1.00 | 23.58 | N |
| ATOM | 14883 | H | ARG | A1225 | 118.961 | 176.288 | 164.573 | 1.00 | 23.58 | H |
| ATOM | 14884 | HA | ARG | A1225 | 117.641 | 174.599 | 164.476 | 1.00 | 23.58 | H |
| ATOM | 14885 | HB2 | ARG | A1225 | 116.814 | 175.720 | 162.033 | 1.00 | 23.58 | H |
| ATOM | 14886 | HB3 | ARG | A1225 | 115.931 | 174.816 | 162.981 | 1.00 | 23.58 | H |
| ATOM | 14887 | HG2 | ARG | A1225 | 115.331 | 176.475 | 164.236 | 1.00 | 23.58 | H |
| ATOM | 14888 | HG3 | ARG | A1225 | 116.729 | 177.224 | 164.087 | 1.00 | 23.58 | H |
| ATOM | 14889 | HD2 | ARG | A1225 | 114.720 | 177.175 | 162.109 | 1.00 | 23.58 | H |
| ATOM | 14890 | HD3 | ARG | A1225 | 115.078 | 178.451 | 162.992 | 1.00 | 23.58 | H |
| ATOM | 14891 | HE | ARG | A1225 | 117.257 | 178.002 | 161.808 | 1.00 | 23.58 | H |
| ATOM | 14892 | HH11 | ARG | A1225 | 114.275 | 178.096 | 160.365 | 1.00 | 23.58 | H |
| ATOM | 14893 | HH12 | ARG | A1225 | 114.768 | 178.531 | 159.030 | 1.00 | 23.58 | H |
| ATOM | 14894 | HH21 | ARG | A1225 | 117.961 | 178.763 | 159.803 | 1.00 | 23.58 | H |
| ATOM | 14895 | HH22 | ARG | A1225 | 116.988 | 178.934 | 158.688 | 1.00 | 23.58 | H |
| ATOM | 14896 | N | HIS | A1226 | 119.328 | 172.985 | 163.277 | 1.00 | 21.12 | N |
| ATOM | 14897 | CA | HIS | A1226 | 119.819 | 171.756 | 162.652 | 1.00 | 21.12 | C |
| ATOM | 14898 | C | HIS | A1226 | 120.754 | 172.054 | 161.484 | 1.00 | 21.12 | C |
| ATOM | 14899 | O | HIS | A1226 | 121.369 | 171.135 | 160.933 | 1.00 | 21.12 | O |
| ATOM | 14900 | CB | HIS | A1226 | 118.667 | 170.856 | 162.173 | 1.00 | 21.12 | C |
| ATOM | 14901 | CG | HIS | A1226 | 118.364 | 170.962 | 160.706 | 1.00 | 21.12 | C |
| ATOM | 14902 | ND1 | HIS | A1226 | 119.172 | 170.401 | 159.739 | 1.00 | 21.12 | N |
| ATOM | 14903 | CD2 | HIS | A1226 | 117.316 | 171.510 | 160.044 | 1.00 | 21.12 | C |
| ATOM | 14904 | CE1 | HIS | A1226 | 118.657 | 170.635 | 158.546 | 1.00 | 21.12 | C |
| ATOM | 14905 | NE2 | HIS | A1226 | 117.531 | 171.306 | 158.703 | 1.00 | 21.12 | N |
| ATOM | 14906 | H | HIS | A1226 | 119.722 | 173.160 | 164.021 | 1.00 | 21.12 | H |
| ATOM | 14907 | HA | HIS | A1226 | 120.326 | 171.257 | 163.311 | 1.00 | 21.12 | H |
| ATOM | 14908 | HB2 | HIS | A1226 | 118.897 | 169.932 | 162.357 | 1.00 | 21.12 | H |
| ATOM | 14909 | HB3 | HIS | A1226 | 117.862 | 171.093 | 162.657 | 1.00 | 21.12 | H |
| ATOM | 14910 | HD2 | HIS | A1226 | 116.599 | 171.963 | 160.425 | 1.00 | 21.12 | H |
| ATOM | 14911 | HE1 | HIS | A1226 | 119.020 | 170.366 | 157.733 | 1.00 | 21.12 | H |
| ATOM | 14912 | HE2 | HIS | A1226 | 117.012 | 171.567 | 158.067 | 1.00 | 21.12 | H |
| ATOM | 14913 | N | TYR | A1227 | 120.880 | 173.326 | 161.101 | 1.00 | 21.95 | N |
| ATOM | 14914 | CA | TYR | A1227 | 122.022 | 173.730 | 160.292 | 1.00 | 21.95 | C |
| ATOM | 14915 | C | TYR | A1227 | 123.251 | 173.946 | 161.162 | 1.00 | 21.95 | C |
| ATOM | 14916 | O | TYR | A1227 | 124.363 | 174.067 | 160.636 | 1.00 | 21.95 | O |
| ATOM | 14917 | CB | TYR | A1227 | 121.704 | 175.002 | 159.501 | 1.00 | 21.95 | C |
| ATOM | 14918 | CG | TYR | A1227 | 120.583 | 174.866 | 158.486 | 1.00 | 21.95 | C |
| ATOM | 14919 | CD1 | TYR | A1227 | 120.838 | 174.400 | 157.202 | 1.00 | 21.95 | C |
| ATOM | 14920 | CD2 | TYR | A1227 | 119.278 | 175.230 | 158.799 | 1.00 | 21.95 | C |
| ATOM | 14921 | CE1 | TYR | A1227 | 119.825 | 174.282 | 156.268 | 1.00 | 21.95 | C |
| ATOM | 14922 | CE2 | TYR | A1227 | 118.259 | 175.116 | 157.869 | 1.00 | 21.95 | C |
| ATOM | 14923 | CZ | TYR | A1227 | 118.539 | 174.643 | 156.606 | 1.00 | 21.95 | C |
| ATOM | 14924 | OH | TYR | A1227 | 117.529 | 174.528 | 155.677 | 1.00 | 21.95 | O |
| ATOM | 14925 | H | TYR | A1227 | 120.332 | 173.960 | 161.287 | 1.00 | 21.95 | H |
| ATOM | 14926 | HA | TYR | A1227 | 122.223 | 173.026 | 159.656 | 1.00 | 21.95 | H |
| ATOM | 14927 | HB2 | TYR | A1227 | 121.455 | 175.702 | 160.124 | 1.00 | 21.95 | H |
| ATOM | 14928 | HB3 | TYR | A1227 | 122.502 | 175.268 | 159.018 | 1.00 | 21.95 | H |
| ATOM | 14929 | HD1 | TYR | A1227 | 121.704 | 174.154 | 156.970 | 1.00 | 21.95 | H |
| ATOM | 14930 | HD2 | TYR | A1227 | 119.081 | 175.550 | 159.647 | 1.00 | 21.95 | H |
| ATOM | 14931 | HE1 | TYR | A1227 | 120.012 | 173.963 | 155.414 | 1.00 | 21.95 | H |
| ATOM | 14932 | HE2 | TYR | A1227 | 117.390 | 175.359 | 158.095 | 1.00 | 21.95 | H |
| ATOM | 14933 | HH | TYR | A1227 | 116.822 | 174.868 | 155.977 | 1.00 | 21.95 | H |
| ATOM | 14934 | N | TYR | A1228 | 123.062 | 173.987 | 162.484 | 1.00 | 14.63 | N |
| ATOM | 14935 | CA | TYR | A1228 | 124.162 | 174.214 | 163.414 | 1.00 | 14.63 | C |
| ATOM | 14936 | C | TYR | A1228 | 125.209 | 173.111 | 163.331 | 1.00 | 14.63 | C |
| ATOM | 14937 | O | TYR | A1228 | 126.413 | 173.379 | 163.408 | 1.00 | 14.63 | O |
| ATOM | 14938 | CB | TYR | A1228 | 123.603 | 174.311 | 164.836 | 1.00 | 14.63 | C |
| ATOM | 14939 | CG | TYR | A1228 | 124.641 | 174.526 | 165.913 | 1.00 | 14.63 | C |
| ATOM | 14940 | CD1 | TYR | A1228 | 125.119 | 175.795 | 166.200 | 1.00 | 14.63 | C |
| ATOM | 14941 | CD2 | TYR | A1228 | 125.136 | 173.457 | 166.649 | 1.00 | 14.63 | C |
| ATOM | 14942 | CE1 | TYR | A1228 | 126.062 | 175.997 | 167.186 | 1.00 | 14.63 | C |
| ATOM | 14943 | CE2 | TYR | A1228 | 126.081 | 173.649 | 167.637 | 1.00 | 14.63 | C |
| ATOM | 14944 | CZ | TYR | A1228 | 126.541 | 174.921 | 167.901 | 1.00 | 14.63 | C |
| ATOM | 14945 | OH | TYR | A1228 | 127.483 | 175.118 | 168.884 | 1.00 | 14.63 | O |
| ATOM | 14946 | H | TYR | A1228 | 122.300 | 173.886 | 162.868 | 1.00 | 14.63 | H |
| ATOM | 14947 | HA | TYR | A1228 | 124.592 | 175.056 | 163.200 | 1.00 | 14.63 | H |
| ATOM | 14948 | HB2 | TYR | A1228 | 122.982 | 175.055 | 164.875 | 1.00 | 14.63 | H |
| ATOM | 14949 | HB3 | TYR | A1228 | 123.134 | 173.486 | 165.041 | 1.00 | 14.63 | H |
| ATOM | 14950 | HD1 | TYR | A1228 | 124.797 | 176.522 | 165.721 | 1.00 | 14.63 | H |
| ATOM | 14951 | HD2 | TYR | A1228 | 124.828 | 172.598 | 166.472 | 1.00 | 14.63 | H |
| ATOM | 14952 | HE1 | TYR | A1228 | 126.374 | 176.854 | 167.367 | 1.00 | 14.63 | H |
| ATOM | 14953 | HE2 | TYR | A1228 | 126.407 | 172.925 | 168.121 | 1.00 | 14.63 | H |
| ATOM | 14954 | HH | TYR | A1228 | 127.682 | 174.384 | 169.241 | 1.00 | 14.63 | H |
| ATOM | 14955 | N | PHE | A1229 | 124.769 | 171.864 | 163.178 | 1.00 | 14.80 | N |
| ATOM | 14956 | CA | PHE | A1229 | 125.660 | 170.715 | 163.278 | 1.00 | 14.80 | C |
| ATOM | 14957 | C | PHE | A1229 | 126.379 | 170.390 | 161.975 | 1.00 | 14.80 | C |
| ATOM | 14958 | O | PHE | A1229 | 127.139 | 169.417 | 161.936 | 1.00 | 14.80 | O |
| ATOM | 14959 | CB | PHE | A1229 | 124.874 | 169.491 | 163.752 | 1.00 | 14.80 | C |
| ATOM | 14960 | CG | PHE | A1229 | 124.277 | 169.653 | 165.119 | 1.00 | 14.80 | C |
| ATOM | 14961 | CD1 | PHE | A1229 | 125.065 | 169.531 | 166.251 | 1.00 | 14.80 | C |
| ATOM | 14962 | CD2 | PHE | A1229 | 122.931 | 169.938 | 165.273 | 1.00 | 14.80 | C |
| ATOM | 14963 | CE1 | PHE | A1229 | 124.520 | 169.684 | 167.511 | 1.00 | 14.80 | C |
| ATOM | 14964 | CE2 | PHE | A1229 | 122.380 | 170.091 | 166.530 | 1.00 | 14.80 | C |
| ATOM | 14965 | CZ | PHE | A1229 | 123.176 | 169.964 | 167.650 | 1.00 | 14.80 | C |
| ATOM | 14966 | H | PHE | A1229 | 123.951 | 171.656 | 163.013 | 1.00 | 14.80 | H |
| ATOM | 14967 | HA | PHE | A1229 | 126.337 | 170.907 | 163.946 | 1.00 | 14.80 | H |
| ATOM | 14968 | HB2 | PHE | A1229 | 124.149 | 169.324 | 163.129 | 1.00 | 14.80 | H |
| ATOM | 14969 | HB3 | PHE | A1229 | 125.470 | 168.726 | 163.777 | 1.00 | 14.80 | H |
| ATOM | 14970 | HD1 | PHE | A1229 | 125.971 | 169.343 | 166.163 | 1.00 | 14.80 | H |
| ATOM | 14971 | HD2 | PHE | A1229 | 122.391 | 170.024 | 164.520 | 1.00 | 14.80 | H |
| ATOM | 14972 | HE1 | PHE | A1229 | 125.058 | 169.598 | 168.265 | 1.00 | 14.80 | H |
| ATOM | 14973 | HE2 | PHE | A1229 | 121.474 | 170.280 | 166.621 | 1.00 | 14.80 | H |
| ATOM | 14974 | HZ | PHE | A1229 | 122.807 | 170.067 | 168.498 | 1.00 | 14.80 | H |
| ATOM | 14975 | N | THR | A1230 | 126.157 | 171.160 | 160.909 | 1.00 | 16.95 | N |
| ATOM | 14976 | CA | THR | A1230 | 126.917 | 170.940 | 159.684 | 1.00 | 16.95 | C |
| ATOM | 14977 | C | THR | A1230 | 128.359 | 171.415 | 159.827 | 1.00 | 16.95 | C |
| ATOM | 14978 | O | THR | A1230 | 129.239 | 170.946 | 159.097 | 1.00 | 16.95 | O |
| ATOM | 14979 | CB | THR | A1230 | 126.242 | 171.645 | 158.507 | 1.00 | 16.95 | C |
| ATOM | 14980 | OG1 | THR | A1230 | 126.177 | 173.055 | 158.760 | 1.00 | 16.95 | O |
| ATOM | 14981 | CG2 | THR | A1230 | 124.836 | 171.100 | 158.290 | 1.00 | 16.95 | C |
| ATOM | 14982 | H | THR | A1230 | 125.585 | 171.801 | 160.869 | 1.00 | 16.95 | H |
| ATOM | 14983 | HA | THR | A1230 | 126.935 | 169.989 | 159.490 | 1.00 | 16.95 | H |
| ATOM | 14984 | HB | THR | A1230 | 126.756 | 171.487 | 157.700 | 1.00 | 16.95 | H |
| ATOM | 14985 | HG1 | THR | A1230 | 125.830 | 173.448 | 158.104 | 1.00 | 16.95 | H |
| ATOM | 14986 | HG21 | THR | A1230 | 124.427 | 171.536 | 157.527 | 1.00 | 16.95 | H |
| ATOM | 14987 | HG22 | THR | A1230 | 124.874 | 170.145 | 158.124 | 1.00 | 16.95 | H |
| ATOM | 14988 | HG23 | THR | A1230 | 124.290 | 171.262 | 159.076 | 1.00 | 16.95 | H |
| ATOM | 14989 | N | VAL | A1231 | 128.618 | 172.332 | 160.756 | 1.00 | 13.54 | N |
| ATOM | 14990 | CA | VAL | A1231 | 129.953 | 172.876 | 160.973 | 1.00 | 13.54 | C |
| ATOM | 14991 | C | VAL | A1231 | 130.625 | 172.096 | 162.097 | 1.00 | 13.54 | C |
| ATOM | 14992 | O | VAL | A1231 | 130.032 | 171.889 | 163.163 | 1.00 | 13.54 | O |
| ATOM | 14993 | CB | VAL | A1231 | 129.884 | 174.376 | 161.307 | 1.00 | 13.54 | C |
| ATOM | 14994 | CG1 | VAL | A1231 | 131.281 | 174.975 | 161.385 | 1.00 | 13.54 | C |
| ATOM | 14995 | CG2 | VAL | A1231 | 129.031 | 175.112 | 160.278 | 1.00 | 13.54 | C |
| ATOM | 14996 | H | VAL | A1231 | 128.023 | 172.662 | 161.283 | 1.00 | 13.54 | H |
| ATOM | 14997 | HA | VAL | A1231 | 130.481 | 172.768 | 160.167 | 1.00 | 13.54 | H |
| ATOM | 14998 | HB | VAL | A1231 | 129.464 | 174.486 | 162.174 | 1.00 | 13.54 | H |
| ATOM | 14999 | HG11 | VAL | A1231 | 131.205 | 175.936 | 161.501 | 1.00 | 13.54 | H |
| ATOM | 15000 | HG12 | VAL | A1231 | 131.752 | 174.588 | 162.139 | 1.00 | 13.54 | H |
| ATOM | 15001 | HG13 | VAL | A1231 | 131.754 | 174.780 | 160.561 | 1.00 | 13.54 | H |
| ATOM | 15002 | HG21 | VAL | A1231 | 129.087 | 176.066 | 160.446 | 1.00 | 13.54 | H |
| ATOM | 15003 | HG22 | VAL | A1231 | 129.364 | 174.912 | 159.390 | 1.00 | 13.54 | H |
| ATOM | 15004 | HG23 | VAL | A1231 | 128.111 | 174.816 | 160.360 | 1.00 | 13.54 | H |
| ATOM | 15005 | N | GLY | A1232 | 131.863 | 171.658 | 161.859 | 1.00 | 11.45 | N |
| ATOM | 15006 | CA | GLY | A1232 | 132.557 | 170.850 | 162.850 | 1.00 | 11.45 | C |
| ATOM | 15007 | C | GLY | A1232 | 132.891 | 171.616 | 164.118 | 1.00 | 11.45 | C |
| ATOM | 15008 | O | GLY | A1232 | 132.763 | 171.086 | 165.227 | 1.00 | 11.45 | O |
| ATOM | 15009 | H | GLY | A1232 | 132.314 | 171.815 | 161.143 | 1.00 | 11.45 | H |
| ATOM | 15010 | HA2 | GLY | A1232 | 132.000 | 170.093 | 163.091 | 1.00 | 11.45 | H |
| ATOM | 15011 | HA3 | GLY | A1232 | 133.383 | 170.512 | 162.470 | 1.00 | 11.45 | H |
| ATOM | 15012 | N | TRP | A1233 | 133.333 | 172.867 | 163.974 | 1.00 | 9.40 | N |
| ATOM | 15013 | CA | TRP | A1233 | 133.640 | 173.672 | 165.151 | 1.00 | 9.40 | C |
| ATOM | 15014 | C | TRP | A1233 | 132.402 | 173.858 | 166.017 | 1.00 | 9.40 | C |
| ATOM | 15015 | O | TRP | A1233 | 132.495 | 173.901 | 167.250 | 1.00 | 9.40 | O |
| ATOM | 15016 | CB | TRP | A1233 | 134.209 | 175.028 | 164.732 | 1.00 | 9.40 | C |
| ATOM | 15017 | CG | TRP | A1233 | 135.703 | 175.037 | 164.583 | 1.00 | 9.40 | C |
| ATOM | 15018 | CD1 | TRP | A1233 | 136.412 | 175.032 | 163.417 | 1.00 | 9.40 | C |
| ATOM | 15019 | CD2 | TRP | A1233 | 136.671 | 175.048 | 165.640 | 1.00 | 9.40 | C |
| ATOM | 15020 | NE1 | TRP | A1233 | 137.759 | 175.044 | 163.682 | 1.00 | 9.40 | N |
| ATOM | 15021 | CE2 | TRP | A1233 | 137.945 | 175.054 | 165.039 | 1.00 | 9.40 | C |
| ATOM | 15022 | CE3 | TRP | A1233 | 136.583 | 175.058 | 167.036 | 1.00 | 9.40 | C |
| ATOM | 15023 | CZ2 | TRP | A1233 | 139.122 | 175.067 | 165.785 | 1.00 | 9.40 | C |
| ATOM | 15024 | CZ3 | TRP | A1233 | 137.753 | 175.071 | 167.774 | 1.00 | 9.40 | C |
| ATOM | 15025 | CH2 | TRP | A1233 | 139.005 | 175.075 | 167.148 | 1.00 | 9.40 | C |
| ATOM | 15026 | H | TRP | A1233 | 133.460 | 173.264 | 163.222 | 1.00 | 9.40 | H |
| ATOM | 15027 | HA | TRP | A1233 | 134.311 | 173.215 | 165.682 | 1.00 | 9.40 | H |
| ATOM | 15028 | HB2 | TRP | A1233 | 133.824 | 175.282 | 163.879 | 1.00 | 9.40 | H |
| ATOM | 15029 | HB3 | TRP | A1233 | 133.973 | 175.682 | 165.406 | 1.00 | 9.40 | H |
| ATOM | 15030 | HD1 | TRP | A1233 | 136.038 | 175.024 | 162.566 | 1.00 | 9.40 | H |
| ATOM | 15031 | HE1 | TRP | A1233 | 138.385 | 175.043 | 163.092 | 1.00 | 9.40 | H |
| ATOM | 15032 | HE3 | TRP | A1233 | 135.755 | 175.056 | 167.458 | 1.00 | 9.40 | H |
| ATOM | 15033 | HZ2 | TRP | A1233 | 139.956 | 175.069 | 165.372 | 1.00 | 9.40 | H |
| ATOM | 15034 | HZ3 | TRP | A1233 | 137.708 | 175.077 | 168.703 | 1.00 | 9.40 | H |
| ATOM | 15035 | HH2 | TRP | A1233 | 139.775 | 175.084 | 167.670 | 1.00 | 9.40 | H |
| ATOM | 15036 | N | ASN | A1234 | 131.231 | 173.972 | 165.388 | 1.00 | 11.66 | N |
| ATOM | 15037 | CA | ASN | A1234 | 129.991 | 174.060 | 166.151 | 1.00 | 11.66 | C |
| ATOM | 15038 | C | ASN | A1234 | 129.776 | 172.803 | 166.984 | 1.00 | 11.66 | C |
| ATOM | 15039 | O | ASN | A1234 | 129.358 | 172.882 | 168.145 | 1.00 | 11.66 | O |
| ATOM | 15040 | CB | ASN | A1234 | 128.814 | 174.288 | 165.203 | 1.00 | 11.66 | C |
| ATOM | 15041 | CG | ASN | A1234 | 128.684 | 175.737 | 164.776 | 1.00 | 11.66 | C |
| ATOM | 15042 | OD1 | ASN | A1234 | 129.596 | 176.538 | 164.976 | 1.00 | 11.66 | O |
| ATOM | 15043 | ND2 | ASN | A1234 | 127.549 | 176.079 | 164.178 | 1.00 | 11.66 | N |
| ATOM | 15044 | H | ASN | A1234 | 131.130 | 174.000 | 164.535 | 1.00 | 11.66 | H |
| ATOM | 15045 | HA | ASN | A1234 | 130.043 | 174.816 | 166.756 | 1.00 | 11.66 | H |
| ATOM | 15046 | HB2 | ASN | A1234 | 128.942 | 173.751 | 164.405 | 1.00 | 11.66 | H |
| ATOM | 15047 | HB3 | ASN | A1234 | 127.993 | 174.031 | 165.648 | 1.00 | 11.66 | H |
| ATOM | 15048 | HD21 | ASN | A1234 | 126.933 | 175.491 | 164.054 | 1.00 | 11.66 | H |
| ATOM | 15049 | HD22 | ASN | A1234 | 127.429 | 176.889 | 163.917 | 1.00 | 11.66 | H |
| ATOM | 15050 | N | ILE | A1235 | 130.068 | 171.634 | 166.412 | 1.00 | 8.89 | N |
| ATOM | 15051 | CA | ILE | A1235 | 129.938 | 170.387 | 167.160 | 1.00 | 8.89 | C |
| ATOM | 15052 | C | ILE | A1235 | 130.919 | 170.364 | 168.325 | 1.00 | 8.89 | C |
| ATOM | 15053 | O | ILE | A1235 | 130.581 | 169.923 | 169.431 | 1.00 | 8.89 | O |
| ATOM | 15054 | CB | ILE | A1235 | 130.144 | 169.179 | 166.227 | 1.00 | 8.89 | C |
| ATOM | 15055 | CG1 | ILE | A1235 | 129.126 | 169.200 | 165.083 | 1.00 | 8.89 | C |
| ATOM | 15056 | CG2 | ILE | A1235 | 130.036 | 167.876 | 167.008 | 1.00 | 8.89 | C |
| ATOM | 15057 | CD1 | ILE | A1235 | 129.514 | 168.322 | 163.913 | 1.00 | 8.89 | C |
| ATOM | 15058 | H | ILE | A1235 | 130.343 | 171.538 | 165.603 | 1.00 | 8.89 | H |
| ATOM | 15059 | HA | ILE | A1235 | 129.041 | 170.332 | 167.524 | 1.00 | 8.89 | H |
| ATOM | 15060 | HB | ILE | A1235 | 131.034 | 169.235 | 165.845 | 1.00 | 8.89 | H |
| ATOM | 15061 | HG12 | ILE | A1235 | 128.271 | 168.888 | 165.420 | 1.00 | 8.89 | H |
| ATOM | 15062 | HG13 | ILE | A1235 | 129.036 | 170.106 | 164.751 | 1.00 | 8.89 | H |
| ATOM | 15063 | HG21 | ILE | A1235 | 129.997 | 167.135 | 166.383 | 1.00 | 8.89 | H |
| ATOM | 15064 | HG22 | ILE | A1235 | 130.814 | 167.781 | 167.579 | 1.00 | 8.89 | H |
| ATOM | 15065 | HG23 | ILE | A1235 | 129.229 | 167.896 | 167.546 | 1.00 | 8.89 | H |
| ATOM | 15066 | HD11 | ILE | A1235 | 128.872 | 168.446 | 163.198 | 1.00 | 8.89 | H |
| ATOM | 15067 | HD12 | ILE | A1235 | 130.399 | 168.575 | 163.610 | 1.00 | 8.89 | H |
| ATOM | 15068 | HD13 | ILE | A1235 | 129.514 | 167.395 | 164.199 | 1.00 | 8.89 | H |
| ATOM | 15069 | N | PHE | A1236 | 132.151 | 170.822 | 168.094 | 1.00 | 7.21 | N |
| ATOM | 15070 | CA | PHE | A1236 | 133.133 | 170.848 | 169.175 | 1.00 | 7.21 | C |
| ATOM | 15071 | C | PHE | A1236 | 132.674 | 171.757 | 170.311 | 1.00 | 7.21 | C |
| ATOM | 15072 | O | PHE | A1236 | 132.779 | 171.392 | 171.489 | 1.00 | 7.21 | O |
| ATOM | 15073 | CB | PHE | A1236 | 134.490 | 171.299 | 168.635 | 1.00 | 7.21 | C |
| ATOM | 15074 | CG | PHE | A1236 | 135.553 | 171.414 | 169.688 | 1.00 | 7.21 | C |
| ATOM | 15075 | CD1 | PHE | A1236 | 136.267 | 170.298 | 170.092 | 1.00 | 7.21 | C |
| ATOM | 15076 | CD2 | PHE | A1236 | 135.845 | 172.636 | 170.270 | 1.00 | 7.21 | C |
| ATOM | 15077 | CE1 | PHE | A1236 | 137.247 | 170.398 | 171.059 | 1.00 | 7.21 | C |
| ATOM | 15078 | CE2 | PHE | A1236 | 136.824 | 172.742 | 171.237 | 1.00 | 7.21 | C |
| ATOM | 15079 | CZ | PHE | A1236 | 137.526 | 171.622 | 171.633 | 1.00 | 7.21 | C |
| ATOM | 15080 | H | PHE | A1236 | 132.436 | 171.118 | 167.339 | 1.00 | 7.21 | H |
| ATOM | 15081 | HA | PHE | A1236 | 133.234 | 169.952 | 169.531 | 1.00 | 7.21 | H |
| ATOM | 15082 | HB2 | PHE | A1236 | 134.793 | 170.656 | 167.975 | 1.00 | 7.21 | H |
| ATOM | 15083 | HB3 | PHE | A1236 | 134.387 | 172.171 | 168.222 | 1.00 | 7.21 | H |
| ATOM | 15084 | HD1 | PHE | A1236 | 136.081 | 169.471 | 169.709 | 1.00 | 7.21 | H |
| ATOM | 15085 | HD2 | PHE | A1236 | 135.374 | 173.394 | 170.007 | 1.00 | 7.21 | H |
| ATOM | 15086 | HE1 | PHE | A1236 | 137.719 | 169.642 | 171.324 | 1.00 | 7.21 | H |
| ATOM | 15087 | HE2 | PHE | A1236 | 137.011 | 173.568 | 171.622 | 1.00 | 7.21 | H |
| ATOM | 15088 | HZ | PHE | A1236 | 138.187 | 171.691 | 172.284 | 1.00 | 7.21 | H |
| ATOM | 15089 | N | ASP | A1237 | 132.150 | 172.938 | 169.976 | 1.00 | 10.05 | N |
| ATOM | 15090 | CA | ASP | A1237 | 131.661 | 173.850 | 171.008 | 1.00 | 10.05 | C |
| ATOM | 15091 | C | ASP | A1237 | 130.464 | 173.260 | 171.744 | 1.00 | 10.05 | C |
| ATOM | 15092 | O | ASP | A1237 | 130.332 | 173.423 | 172.962 | 1.00 | 10.05 | O |
| ATOM | 15093 | CB | ASP | A1237 | 131.300 | 175.202 | 170.391 | 1.00 | 10.05 | C |
| ATOM | 15094 | CG | ASP | A1237 | 132.332 | 175.682 | 169.390 | 1.00 | 10.05 | C |
| ATOM | 15095 | OD1 | ASP | A1237 | 133.470 | 175.167 | 169.416 | 1.00 | 10.05 | O |
| ATOM | 15096 | OD2 | ASP | A1237 | 132.007 | 176.571 | 168.575 | 1.00 | 10.05 | O |
| ATOM | 15097 | H | ASP | A1237 | 132.064 | 173.225 | 169.170 | 1.00 | 10.05 | H |
| ATOM | 15098 | HA | ASP | A1237 | 132.367 | 173.997 | 171.656 | 1.00 | 10.05 | H |
| ATOM | 15099 | HB2 | ASP | A1237 | 130.450 | 175.123 | 169.930 | 1.00 | 10.05 | H |
| ATOM | 15100 | HB3 | ASP | A1237 | 131.235 | 175.865 | 171.096 | 1.00 | 10.05 | H |
| ATOM | 15101 | N | PHE | A1238 | 129.575 | 172.578 | 171.019 | 1.00 | 8.65 | N |
| ATOM | 15102 | CA | PHE | A1238 | 128.428 | 171.932 | 171.650 | 1.00 | 8.65 | C |
| ATOM | 15103 | C | PHE | A1238 | 128.881 | 170.874 | 172.652 | 1.00 | 8.65 | C |
| ATOM | 15104 | O | PHE | A1238 | 128.372 | 170.801 | 173.781 | 1.00 | 8.65 | O |
| ATOM | 15105 | CB | PHE | A1238 | 127.548 | 171.319 | 170.558 | 1.00 | 8.65 | C |
| ATOM | 15106 | CG | PHE | A1238 | 126.298 | 170.667 | 171.066 | 1.00 | 8.65 | C |
| ATOM | 15107 | CD1 | PHE | A1238 | 125.231 | 171.430 | 171.505 | 1.00 | 8.65 | C |
| ATOM | 15108 | CD2 | PHE | A1238 | 126.180 | 169.288 | 171.077 | 1.00 | 8.65 | C |
| ATOM | 15109 | CE1 | PHE | A1238 | 124.075 | 170.829 | 171.962 | 1.00 | 8.65 | C |
| ATOM | 15110 | CE2 | PHE | A1238 | 125.027 | 168.681 | 171.530 | 1.00 | 8.65 | C |
| ATOM | 15111 | CZ | PHE | A1238 | 123.972 | 169.454 | 171.974 | 1.00 | 8.65 | C |
| ATOM | 15112 | H | PHE | A1238 | 129.615 | 172.477 | 170.166 | 1.00 | 8.65 | H |
| ATOM | 15113 | HA | PHE | A1238 | 127.906 | 172.597 | 172.125 | 1.00 | 8.65 | H |
| ATOM | 15114 | HB2 | PHE | A1238 | 127.285 | 172.021 | 169.942 | 1.00 | 8.65 | H |
| ATOM | 15115 | HB3 | PHE | A1238 | 128.062 | 170.645 | 170.086 | 1.00 | 8.65 | H |
| ATOM | 15116 | HD1 | PHE | A1238 | 125.296 | 172.358 | 171.499 | 1.00 | 8.65 | H |
| ATOM | 15117 | HD2 | PHE | A1238 | 126.889 | 168.763 | 170.779 | 1.00 | 8.65 | H |
| ATOM | 15118 | HE1 | PHE | A1238 | 123.365 | 171.351 | 172.260 | 1.00 | 8.65 | H |
| ATOM | 15119 | HE2 | PHE | A1238 | 124.961 | 167.754 | 171.538 | 1.00 | 8.65 | H |
| ATOM | 15120 | HZ | PHE | A1238 | 123.195 | 169.047 | 172.282 | 1.00 | 8.65 | H |
| ATOM | 15121 | N | VAL | A1239 | 129.852 | 170.049 | 172.253 | 1.00 | 6.89 | N |
| ATOM | 15122 | CA | VAL | A1239 | 130.386 | 169.031 | 173.152 | 1.00 | 6.89 | C |
| ATOM | 15123 | C | VAL | A1239 | 131.019 | 169.685 | 174.371 | 1.00 | 6.89 | C |
| ATOM | 15124 | O | VAL | A1239 | 130.845 | 169.221 | 175.506 | 1.00 | 6.89 | O |
| ATOM | 15125 | CB | VAL | A1239 | 131.392 | 168.135 | 172.406 | 1.00 | 6.89 | C |
| ATOM | 15126 | CG1 | VAL | A1239 | 132.161 | 167.259 | 173.386 | 1.00 | 6.89 | C |
| ATOM | 15127 | CG2 | VAL | A1239 | 130.677 | 167.281 | 171.370 | 1.00 | 6.89 | C |
| ATOM | 15128 | H | VAL | A1239 | 130.215 | 170.061 | 171.473 | 1.00 | 6.89 | H |
| ATOM | 15129 | HA | VAL | A1239 | 129.657 | 168.469 | 173.459 | 1.00 | 6.89 | H |
| ATOM | 15130 | HB | VAL | A1239 | 132.031 | 168.697 | 171.941 | 1.00 | 6.89 | H |
| ATOM | 15131 | HG11 | VAL | A1239 | 132.550 | 166.514 | 172.902 | 1.00 | 6.89 | H |
| ATOM | 15132 | HG12 | VAL | A1239 | 132.864 | 167.786 | 173.798 | 1.00 | 6.89 | H |
| ATOM | 15133 | HG13 | VAL | A1239 | 131.550 | 166.929 | 174.063 | 1.00 | 6.89 | H |
| ATOM | 15134 | HG21 | VAL | A1239 | 131.334 | 166.750 | 170.892 | 1.00 | 6.89 | H |
| ATOM | 15135 | HG22 | VAL | A1239 | 130.047 | 166.699 | 171.822 | 1.00 | 6.89 | H |
| ATOM | 15136 | HG23 | VAL | A1239 | 130.207 | 167.862 | 170.752 | 1.00 | 6.89 | H |
| ATOM | 15137 | N | VAL | A1240 | 131.769 | 170.769 | 174.158 | 1.00 | 6.57 | N |
| ATOM | 15138 | CA | VAL | A1240 | 132.418 | 171.451 | 175.274 | 1.00 | 6.57 | C |
| ATOM | 15139 | C | VAL | A1240 | 131.376 | 171.994 | 176.244 | 1.00 | 6.57 | C |
| ATOM | 15140 | O | VAL | A1240 | 131.548 | 171.918 | 177.464 | 1.00 | 6.57 | O |
| ATOM | 15141 | CB | VAL | A1240 | 133.345 | 172.566 | 174.754 | 1.00 | 6.57 | C |
| ATOM | 15142 | CG1 | VAL | A1240 | 133.720 | 173.519 | 175.881 | 1.00 | 6.57 | C |
| ATOM | 15143 | CG2 | VAL | A1240 | 134.591 | 171.965 | 174.123 | 1.00 | 6.57 | C |
| ATOM | 15144 | H | VAL | A1240 | 131.915 | 171.123 | 173.388 | 1.00 | 6.57 | H |
| ATOM | 15145 | HA | VAL | A1240 | 132.965 | 170.811 | 175.755 | 1.00 | 6.57 | H |
| ATOM | 15146 | HB | VAL | A1240 | 132.878 | 173.074 | 174.073 | 1.00 | 6.57 | H |
| ATOM | 15147 | HG11 | VAL | A1240 | 134.505 | 174.023 | 175.615 | 1.00 | 6.57 | H |
| ATOM | 15148 | HG12 | VAL | A1240 | 132.981 | 174.126 | 176.046 | 1.00 | 6.57 | H |
| ATOM | 15149 | HG13 | VAL | A1240 | 133.915 | 173.004 | 176.679 | 1.00 | 6.57 | H |
| ATOM | 15150 | HG21 | VAL | A1240 | 135.142 | 172.681 | 173.770 | 1.00 | 6.57 | H |
| ATOM | 15151 | HG22 | VAL | A1240 | 135.082 | 171.473 | 174.801 | 1.00 | 6.57 | H |
| ATOM | 15152 | HG23 | VAL | A1240 | 134.326 | 171.367 | 173.407 | 1.00 | 6.57 | H |
| ATOM | 15153 | N | VAL | A1241 | 130.285 | 172.558 | 175.721 | 1.00 | 7.29 | N |
| ATOM | 15154 | CA | VAL | A1241 | 129.245 | 173.112 | 176.586 | 1.00 | 7.29 | C |
| ATOM | 15155 | C | VAL | A1241 | 128.587 | 172.010 | 177.410 | 1.00 | 7.29 | C |
| ATOM | 15156 | O | VAL | A1241 | 128.350 | 172.168 | 178.618 | 1.00 | 7.29 | O |
| ATOM | 15157 | CB | VAL | A1241 | 128.210 | 173.883 | 175.745 | 1.00 | 7.29 | C |
| ATOM | 15158 | CG1 | VAL | A1241 | 126.965 | 174.184 | 176.568 | 1.00 | 7.29 | C |
| ATOM | 15159 | CG2 | VAL | A1241 | 128.819 | 175.166 | 175.203 | 1.00 | 7.29 | C |
| ATOM | 15160 | H | VAL | A1241 | 130.126 | 172.630 | 174.879 | 1.00 | 7.29 | H |
| ATOM | 15161 | HA | VAL | A1241 | 129.653 | 173.740 | 177.203 | 1.00 | 7.29 | H |
| ATOM | 15162 | HB | VAL | A1241 | 127.946 | 173.334 | 174.990 | 1.00 | 7.29 | H |
| ATOM | 15163 | HG11 | VAL | A1241 | 126.467 | 174.893 | 176.133 | 1.00 | 7.29 | H |
| ATOM | 15164 | HG12 | VAL | A1241 | 126.418 | 173.385 | 176.624 | 1.00 | 7.29 | H |
| ATOM | 15165 | HG13 | VAL | A1241 | 127.234 | 174.468 | 177.456 | 1.00 | 7.29 | H |
| ATOM | 15166 | HG21 | VAL | A1241 | 128.184 | 175.587 | 174.603 | 1.00 | 7.29 | H |
| ATOM | 15167 | HG22 | VAL | A1241 | 129.018 | 175.759 | 175.944 | 1.00 | 7.29 | H |
| ATOM | 15168 | HG23 | VAL | A1241 | 129.634 | 174.951 | 174.724 | 1.00 | 7.29 | H |
| ATOM | 15169 | N | ILE | A1242 | 128.267 | 170.881 | 176.772 | 1.00 | 7.62 | N |
| ATOM | 15170 | CA | ILE | A1242 | 127.659 | 169.774 | 177.509 | 1.00 | 7.62 | C |
| ATOM | 15171 | C | ILE | A1242 | 128.606 | 169.287 | 178.600 | 1.00 | 7.62 | C |
| ATOM | 15172 | O | ILE | A1242 | 128.199 | 169.052 | 179.749 | 1.00 | 7.62 | O |
| ATOM | 15173 | CB | ILE | A1242 | 127.271 | 168.635 | 176.548 | 1.00 | 7.62 | C |
| ATOM | 15174 | CG1 | ILE | A1242 | 125.909 | 168.920 | 175.916 | 1.00 | 7.62 | C |
| ATOM | 15175 | CG2 | ILE | A1242 | 127.242 | 167.294 | 177.278 | 1.00 | 7.62 | C |
| ATOM | 15176 | CD1 | ILE | A1242 | 125.667 | 168.162 | 174.638 | 1.00 | 7.62 | C |
| ATOM | 15177 | H | ILE | A1242 | 128.387 | 170.735 | 175.933 | 1.00 | 7.62 | H |
| ATOM | 15178 | HA | ILE | A1242 | 126.848 | 170.090 | 177.938 | 1.00 | 7.62 | H |
| ATOM | 15179 | HB | ILE | A1242 | 127.935 | 168.588 | 175.842 | 1.00 | 7.62 | H |
| ATOM | 15180 | HG12 | ILE | A1242 | 125.214 | 168.672 | 176.546 | 1.00 | 7.62 | H |
| ATOM | 15181 | HG13 | ILE | A1242 | 125.849 | 169.868 | 175.716 | 1.00 | 7.62 | H |
| ATOM | 15182 | HG21 | ILE | A1242 | 126.767 | 166.650 | 176.729 | 1.00 | 7.62 | H |
| ATOM | 15183 | HG22 | ILE | A1242 | 128.150 | 166.987 | 177.425 | 1.00 | 7.62 | H |
| ATOM | 15184 | HG23 | ILE | A1242 | 126.781 | 167.401 | 178.125 | 1.00 | 7.62 | H |
| ATOM | 15185 | HD11 | ILE | A1242 | 124.760 | 168.331 | 174.341 | 1.00 | 7.62 | H |
| ATOM | 15186 | HD12 | ILE | A1242 | 126.301 | 168.465 | 173.969 | 1.00 | 7.62 | H |
| ATOM | 15187 | HD13 | ILE | A1242 | 125.788 | 167.214 | 174.803 | 1.00 | 7.62 | H |
| ATOM | 15188 | N | ILE | A1243 | 129.884 | 169.123 | 178.253 | 1.00 | 8.15 | N |
| ATOM | 15189 | CA | ILE | A1243 | 130.865 | 168.669 | 179.232 | 1.00 | 8.15 | C |
| ATOM | 15190 | C | ILE | A1243 | 130.969 | 169.669 | 180.373 | 1.00 | 8.15 | C |
| ATOM | 15191 | O | ILE | A1243 | 131.125 | 169.286 | 181.534 | 1.00 | 8.15 | O |
| ATOM | 15192 | CB | ILE | A1243 | 132.230 | 168.438 | 178.559 | 1.00 | 8.15 | C |
| ATOM | 15193 | CG1 | ILE | A1243 | 132.184 | 167.192 | 177.673 | 1.00 | 8.15 | C |
| ATOM | 15194 | CG2 | ILE | A1243 | 133.323 | 168.295 | 179.607 | 1.00 | 8.15 | C |
| ATOM | 15195 | CD1 | ILE | A1243 | 133.202 | 167.207 | 176.556 | 1.00 | 8.15 | C |
| ATOM | 15196 | H | ILE | A1243 | 130.203 | 169.266 | 177.467 | 1.00 | 8.15 | H |
| ATOM | 15197 | HA | ILE | A1243 | 130.569 | 167.824 | 179.603 | 1.00 | 8.15 | H |
| ATOM | 15198 | HB | ILE | A1243 | 132.434 | 169.205 | 178.002 | 1.00 | 8.15 | H |
| ATOM | 15199 | HG12 | ILE | A1243 | 132.356 | 166.411 | 178.222 | 1.00 | 8.15 | H |
| ATOM | 15200 | HG13 | ILE | A1243 | 131.303 | 167.125 | 177.271 | 1.00 | 8.15 | H |
| ATOM | 15201 | HG21 | ILE | A1243 | 134.094 | 167.869 | 179.200 | 1.00 | 8.15 | H |
| ATOM | 15202 | HG22 | ILE | A1243 | 133.568 | 169.175 | 179.935 | 1.00 | 8.15 | H |
| ATOM | 15203 | HG23 | ILE | A1243 | 132.992 | 167.747 | 180.335 | 1.00 | 8.15 | H |
| ATOM | 15204 | HD11 | ILE | A1243 | 133.051 | 166.441 | 175.980 | 1.00 | 8.15 | H |
| ATOM | 15205 | HD12 | ILE | A1243 | 133.102 | 168.028 | 176.049 | 1.00 | 8.15 | H |
| ATOM | 15206 | HD13 | ILE | A1243 | 134.092 | 167.163 | 176.940 | 1.00 | 8.15 | H |
| ATOM | 15207 | N | SER | A1244 | 130.888 | 170.964 | 180.062 | 1.00 | 8.76 | N |
| ATOM | 15208 | CA | SER | A1244 | 130.985 | 171.982 | 181.102 | 1.00 | 8.76 | C |
| ATOM | 15209 | C | SER | A1244 | 129.810 | 171.901 | 182.067 | 1.00 | 8.76 | C |
| ATOM | 15210 | O | SER | A1244 | 129.993 | 172.020 | 183.282 | 1.00 | 8.76 | O |
| ATOM | 15211 | CB | SER | A1244 | 131.061 | 173.371 | 180.468 | 1.00 | 8.76 | C |
| ATOM | 15212 | OG | SER | A1244 | 132.214 | 173.497 | 179.655 | 1.00 | 8.76 | O |
| ATOM | 15213 | H | SER | A1244 | 130.779 | 171.273 | 179.267 | 1.00 | 8.76 | H |
| ATOM | 15214 | HA | SER | A1244 | 131.799 | 171.840 | 181.609 | 1.00 | 8.76 | H |
| ATOM | 15215 | HB2 | SER | A1244 | 130.272 | 173.512 | 179.921 | 1.00 | 8.76 | H |
| ATOM | 15216 | HB3 | SER | A1244 | 131.098 | 174.037 | 181.172 | 1.00 | 8.76 | H |
| ATOM | 15217 | HG | SER | A1244 | 132.236 | 174.260 | 179.305 | 1.00 | 8.76 | H |
| ATOM | 15218 | N | ILE | A1245 | 128.596 | 171.710 | 181.549 | 1.00 | 8.47 | N |
| ATOM | 15219 | CA | ILE | A1245 | 127.435 | 171.604 | 182.434 | 1.00 | 8.47 | C |
| ATOM | 15220 | C | ILE | A1245 | 127.560 | 170.371 | 183.326 | 1.00 | 8.47 | C |
| ATOM | 15221 | O | ILE | A1245 | 127.370 | 170.436 | 184.554 | 1.00 | 8.47 | O |
| ATOM | 15222 | CB | ILE | A1245 | 126.134 | 171.577 | 181.612 | 1.00 | 8.47 | C |
| ATOM | 15223 | CG1 | ILE | A1245 | 125.874 | 172.949 | 180.987 | 1.00 | 8.47 | C |
| ATOM | 15224 | CG2 | ILE | A1245 | 124.957 | 171.163 | 182.487 | 1.00 | 8.47 | C |
| ATOM | 15225 | CD1 | ILE | A1245 | 124.911 | 172.917 | 179.821 | 1.00 | 8.47 | C |
| ATOM | 15226 | H | ILE | A1245 | 128.420 | 171.640 | 180.711 | 1.00 | 8.47 | H |
| ATOM | 15227 | HA | ILE | A1245 | 127.406 | 172.385 | 183.009 | 1.00 | 8.47 | H |
| ATOM | 15228 | HB | ILE | A1245 | 126.232 | 170.926 | 180.899 | 1.00 | 8.47 | H |
| ATOM | 15229 | HG12 | ILE | A1245 | 125.500 | 173.534 | 181.664 | 1.00 | 8.47 | H |
| ATOM | 15230 | HG13 | ILE | A1245 | 126.714 | 173.312 | 180.668 | 1.00 | 8.47 | H |
| ATOM | 15231 | HG21 | ILE | A1245 | 124.132 | 171.425 | 182.050 | 1.00 | 8.47 | H |
| ATOM | 15232 | HG22 | ILE | A1245 | 124.973 | 170.201 | 182.609 | 1.00 | 8.47 | H |
| ATOM | 15233 | HG23 | ILE | A1245 | 125.030 | 171.610 | 183.345 | 1.00 | 8.47 | H |
| ATOM | 15234 | HD11 | ILE | A1245 | 124.882 | 173.797 | 179.413 | 1.00 | 8.47 | H |
| ATOM | 15235 | HD12 | ILE | A1245 | 125.220 | 172.263 | 179.175 | 1.00 | 8.47 | H |
| ATOM | 15236 | HD13 | ILE | A1245 | 124.030 | 172.673 | 180.144 | 1.00 | 8.47 | H |
| ATOM | 15237 | N | VAL | A1246 | 127.893 | 169.228 | 182.720 | 1.00 | 10.08 | N |
| ATOM | 15238 | CA | VAL | A1246 | 128.034 | 168.001 | 183.501 | 1.00 | 10.08 | C |
| ATOM | 15239 | C | VAL | A1246 | 129.134 | 168.163 | 184.541 | 1.00 | 10.08 | C |
| ATOM | 15240 | O | VAL | A1246 | 129.013 | 167.692 | 185.678 | 1.00 | 10.08 | O |
| ATOM | 15241 | CB | VAL | A1246 | 128.303 | 166.802 | 182.573 | 1.00 | 10.08 | C |
| ATOM | 15242 | CG1 | VAL | A1246 | 128.570 | 165.545 | 183.390 | 1.00 | 10.08 | C |
| ATOM | 15243 | CG2 | VAL | A1246 | 127.129 | 166.589 | 181.630 | 1.00 | 10.08 | C |
| ATOM | 15244 | H | VAL | A1246 | 128.038 | 169.138 | 181.877 | 1.00 | 10.08 | H |
| ATOM | 15245 | HA | VAL | A1246 | 127.203 | 167.832 | 183.971 | 1.00 | 10.08 | H |
| ATOM | 15246 | HB | VAL | A1246 | 129.090 | 166.986 | 182.037 | 1.00 | 10.08 | H |
| ATOM | 15247 | HG11 | VAL | A1246 | 128.505 | 164.773 | 182.807 | 1.00 | 10.08 | H |
| ATOM | 15248 | HG12 | VAL | A1246 | 129.460 | 165.595 | 183.771 | 1.00 | 10.08 | H |
| ATOM | 15249 | HG13 | VAL | A1246 | 127.908 | 165.481 | 184.096 | 1.00 | 10.08 | H |
| ATOM | 15250 | HG21 | VAL | A1246 | 127.315 | 165.825 | 181.063 | 1.00 | 10.08 | H |
| ATOM | 15251 | HG22 | VAL | A1246 | 126.330 | 166.425 | 182.154 | 1.00 | 10.08 | H |
| ATOM | 15252 | HG23 | VAL | A1246 | 127.010 | 167.383 | 181.086 | 1.00 | 10.08 | H |
| ATOM | 15253 | N | GLY | A1247 | 130.225 | 168.833 | 184.167 | 1.00 | 10.63 | N |
| ATOM | 15254 | CA | GLY | A1247 | 131.317 | 169.041 | 185.097 | 1.00 | 10.63 | C |
| ATOM | 15255 | C | GLY | A1247 | 130.956 | 169.973 | 186.233 | 1.00 | 10.63 | C |
| ATOM | 15256 | O | GLY | A1247 | 131.418 | 169.793 | 187.358 | 1.00 | 10.63 | O |
| ATOM | 15257 | H | GLY | A1247 | 130.350 | 169.171 | 183.387 | 1.00 | 10.63 | H |
| ATOM | 15258 | HA2 | GLY | A1247 | 131.596 | 168.190 | 185.469 | 1.00 | 10.63 | H |
| ATOM | 15259 | HA3 | GLY | A1247 | 132.059 | 169.429 | 184.619 | 1.00 | 10.63 | H |
| ATOM | 15260 | N | MET | A1248 | 130.153 | 170.999 | 185.954 | 1.00 | 10.80 | N |
| ATOM | 15261 | CA | MET | A1248 | 129.682 | 171.864 | 187.029 | 1.00 | 10.80 | C |
| ATOM | 15262 | C | MET | A1248 | 128.869 | 171.062 | 188.033 | 1.00 | 10.80 | C |
| ATOM | 15263 | O | MET | A1248 | 129.064 | 171.180 | 189.250 | 1.00 | 10.80 | O |
| ATOM | 15264 | CB | MET | A1248 | 128.850 | 173.015 | 186.463 | 1.00 | 10.80 | C |
| ATOM | 15265 | CG | MET | A1248 | 129.646 | 174.090 | 185.729 | 1.00 | 10.80 | C |
| ATOM | 15266 | SD | MET | A1248 | 130.719 | 175.088 | 186.784 | 1.00 | 10.80 | S |
| ATOM | 15267 | CE | MET | A1248 | 132.247 | 174.145 | 186.740 | 1.00 | 10.80 | C |
| ATOM | 15268 | H | MET | A1248 | 129.876 | 171.213 | 185.168 | 1.00 | 10.80 | H |
| ATOM | 15269 | HA | MET | A1248 | 130.440 | 172.240 | 187.494 | 1.00 | 10.80 | H |
| ATOM | 15270 | HB2 | MET | A1248 | 128.206 | 172.648 | 185.837 | 1.00 | 10.80 | H |
| ATOM | 15271 | HB3 | MET | A1248 | 128.381 | 173.444 | 187.195 | 1.00 | 10.80 | H |
| ATOM | 15272 | HG2 | MET | A1248 | 130.204 | 173.664 | 185.061 | 1.00 | 10.80 | H |
| ATOM | 15273 | HG3 | MET | A1248 | 129.021 | 174.694 | 185.295 | 1.00 | 10.80 | H |
| ATOM | 15274 | HE1 | MET | A1248 | 132.943 | 174.660 | 187.175 | 1.00 | 10.80 | H |
| ATOM | 15275 | HE2 | MET | A1248 | 132.135 | 173.302 | 187.201 | 1.00 | 10.80 | H |
| ATOM | 15276 | HE3 | MET | A1248 | 132.485 | 173.987 | 185.813 | 1.00 | 10.80 | H |
| ATOM | 15277 | N | PHE | A1249 | 127.964 | 170.215 | 187.536 | 1.00 | 14.74 | N |
| ATOM | 15278 | CA | PHE | A1249 | 127.198 | 169.363 | 188.443 | 1.00 | 14.74 | C |
| ATOM | 15279 | C | PHE | A1249 | 128.120 | 168.449 | 189.247 | 1.00 | 14.74 | C |
| ATOM | 15280 | O | PHE | A1249 | 127.993 | 168.334 | 190.476 | 1.00 | 14.74 | O |
| ATOM | 15281 | CB | PHE | A1249 | 126.179 | 168.541 | 187.655 | 1.00 | 14.74 | C |
| ATOM | 15282 | CG | PHE | A1249 | 124.865 | 169.239 | 187.449 | 1.00 | 14.74 | C |
| ATOM | 15283 | CD1 | PHE | A1249 | 123.999 | 169.436 | 188.511 | 1.00 | 14.74 | C |
| ATOM | 15284 | CD2 | PHE | A1249 | 124.493 | 169.692 | 186.194 | 1.00 | 14.74 | C |
| ATOM | 15285 | CE1 | PHE | A1249 | 122.788 | 170.075 | 188.327 | 1.00 | 14.74 | C |
| ATOM | 15286 | CE2 | PHE | A1249 | 123.282 | 170.331 | 186.004 | 1.00 | 14.74 | C |
| ATOM | 15287 | CZ | PHE | A1249 | 122.429 | 170.523 | 187.073 | 1.00 | 14.74 | C |
| ATOM | 15288 | H | PHE | A1249 | 127.778 | 170.120 | 186.702 | 1.00 | 14.74 | H |
| ATOM | 15289 | HA | PHE | A1249 | 126.714 | 169.924 | 189.069 | 1.00 | 14.74 | H |
| ATOM | 15290 | HB2 | PHE | A1249 | 126.549 | 168.343 | 186.780 | 1.00 | 14.74 | H |
| ATOM | 15291 | HB3 | PHE | A1249 | 126.005 | 167.715 | 188.134 | 1.00 | 14.74 | H |
| ATOM | 15292 | HD1 | PHE | A1249 | 124.236 | 169.137 | 189.358 | 1.00 | 14.74 | H |
| ATOM | 15293 | HD2 | PHE | A1249 | 125.064 | 169.565 | 185.471 | 1.00 | 14.74 | H |
| ATOM | 15294 | HE1 | PHE | A1249 | 122.215 | 170.203 | 189.049 | 1.00 | 14.74 | H |
| ATOM | 15295 | HE2 | PHE | A1249 | 123.042 | 170.633 | 185.157 | 1.00 | 14.74 | H |
| ATOM | 15296 | HZ | PHE | A1249 | 121.614 | 170.953 | 186.947 | 1.00 | 14.74 | H |
| ATOM | 15297 | N | LEU | A1250 | 129.068 | 167.801 | 188.568 | 1.00 | 17.81 | N |
| ATOM | 15298 | CA | LEU | A1250 | 129.948 | 166.854 | 189.246 | 1.00 | 17.81 | C |
| ATOM | 15299 | C | LEU | A1250 | 130.825 | 167.551 | 190.278 | 1.00 | 17.81 | C |
| ATOM | 15300 | O | LEU | A1250 | 131.081 | 167.001 | 191.351 | 1.00 | 17.81 | O |
| ATOM | 15301 | CB | LEU | A1250 | 130.813 | 166.113 | 188.226 | 1.00 | 17.81 | C |
| ATOM | 15302 | CG | LEU | A1250 | 131.434 | 164.775 | 188.647 | 1.00 | 17.81 | C |
| ATOM | 15303 | CD1 | LEU | A1250 | 130.456 | 163.901 | 189.425 | 1.00 | 17.81 | C |
| ATOM | 15304 | CD2 | LEU | A1250 | 131.974 | 164.030 | 187.440 | 1.00 | 17.81 | C |
| ATOM | 15305 | H | LEU | A1250 | 129.220 | 167.891 | 187.727 | 1.00 | 17.81 | H |
| ATOM | 15306 | HA | LEU | A1250 | 129.396 | 166.209 | 189.714 | 1.00 | 17.81 | H |
| ATOM | 15307 | N | ALA | A1251 | 131.316 | 168.750 | 189.962 | 1.00 | 16.97 | N |
| ATOM | 15308 | CA | ALA | A1251 | 132.136 | 169.493 | 190.910 | 1.00 | 16.97 | C |
| ATOM | 15309 | C | ALA | A1251 | 131.319 | 169.926 | 192.117 | 1.00 | 16.97 | C |
| ATOM | 15310 | O | ALA | A1251 | 131.796 | 169.851 | 193.256 | 1.00 | 16.97 | O |
| ATOM | 15311 | CB | ALA | A1251 | 132.762 | 170.706 | 190.222 | 1.00 | 16.97 | C |
| ATOM | 15312 | H | ALA | A1251 | 131.187 | 169.150 | 189.211 | 1.00 | 16.97 | H |
| ATOM | 15313 | HA | ALA | A1251 | 132.855 | 168.922 | 191.222 | 1.00 | 16.97 | H |
| ATOM | 15314 | HB1 | ALA | A1251 | 133.306 | 171.188 | 190.864 | 1.00 | 16.97 | H |
| ATOM | 15315 | HB2 | ALA | A1251 | 133.312 | 170.401 | 189.484 | 1.00 | 16.97 | H |
| ATOM | 15316 | HB3 | ALA | A1251 | 132.054 | 171.280 | 189.891 | 1.00 | 16.97 | H |
| ATOM | 15317 | N | ASP | A1252 | 130.083 | 170.382 | 191.892 | 1.00 | 20.66 | N |
| ATOM | 15318 | CA | ASP | A1252 | 129.217 | 170.708 | 193.018 | 1.00 | 20.66 | C |
| ATOM | 15319 | C | ASP | A1252 | 129.030 | 169.496 | 193.918 | 1.00 | 20.66 | C |
| ATOM | 15320 | O | ASP | A1252 | 129.030 | 169.618 | 195.149 | 1.00 | 20.66 | O |
| ATOM | 15321 | CB | ASP | A1252 | 127.866 | 171.214 | 192.512 | 1.00 | 20.66 | C |
| ATOM | 15322 | CG | ASP | A1252 | 127.016 | 171.818 | 193.616 | 1.00 | 20.66 | C |
| ATOM | 15323 | OD1 | ASP | A1252 | 126.889 | 171.190 | 194.689 | 1.00 | 20.66 | O |
| ATOM | 15324 | OD2 | ASP | A1252 | 126.473 | 172.924 | 193.411 | 1.00 | 20.66 | O |
| ATOM | 15325 | H | ASP | A1252 | 129.734 | 170.506 | 191.116 | 1.00 | 20.66 | H |
| ATOM | 15326 | HA | ASP | A1252 | 129.628 | 171.413 | 193.542 | 1.00 | 20.66 | H |
| ATOM | 15327 | HB2 | ASP | A1252 | 128.013 | 171.894 | 191.837 | 1.00 | 20.66 | H |
| ATOM | 15328 | HB3 | ASP | A1252 | 127.374 | 170.470 | 192.130 | 1.00 | 20.66 | H |
| ATOM | 15329 | N | LEU | A1253 | 128.874 | 168.313 | 193.321 | 1.00 | 24.50 | N |
| ATOM | 15330 | CA | LEU | A1253 | 128.697 | 167.108 | 194.127 | 1.00 | 24.50 | C |
| ATOM | 15331 | C | LEU | A1253 | 129.984 | 166.725 | 194.854 | 1.00 | 24.50 | C |
| ATOM | 15332 | O | LEU | A1253 | 129.947 | 166.325 | 196.022 | 1.00 | 24.50 | O |
| ATOM | 15333 | CB | LEU | A1253 | 128.213 | 165.958 | 193.241 | 1.00 | 24.50 | C |
| ATOM | 15334 | CG | LEU | A1253 | 128.102 | 164.574 | 193.891 | 1.00 | 24.50 | C |
| ATOM | 15335 | CD1 | LEU | A1253 | 127.006 | 163.767 | 193.214 | 1.00 | 24.50 | C |
| ATOM | 15336 | CD2 | LEU | A1253 | 129.424 | 163.814 | 193.842 | 1.00 | 24.50 | C |
| ATOM | 15337 | H | LEU | A1253 | 128.868 | 168.184 | 192.471 | 1.00 | 24.50 | H |
| ATOM | 15338 | HA | LEU | A1253 | 128.015 | 167.275 | 194.796 | 1.00 | 24.50 | H |
| ATOM | 15339 | HB2 | LEU | A1253 | 127.331 | 166.188 | 192.910 | 1.00 | 24.50 | H |
| ATOM | 15340 | HB3 | LEU | A1253 | 128.822 | 165.879 | 192.490 | 1.00 | 24.50 | H |
| ATOM | 15341 | HG | LEU | A1253 | 127.857 | 164.685 | 194.823 | 1.00 | 24.50 | H |
| ATOM | 15342 | HD11 | LEU | A1253 | 126.942 | 162.899 | 193.643 | 1.00 | 24.50 | H |
| ATOM | 15343 | HD12 | LEU | A1253 | 126.164 | 164.241 | 193.303 | 1.00 | 24.50 | H |
| ATOM | 15344 | HD13 | LEU | A1253 | 127.228 | 163.658 | 192.276 | 1.00 | 24.50 | H |
| ATOM | 15345 | HD21 | LEU | A1253 | 129.240 | 162.862 | 193.860 | 1.00 | 24.50 | H |
| ATOM | 15346 | HD22 | LEU | A1253 | 129.895 | 164.042 | 193.026 | 1.00 | 24.50 | H |
| ATOM | 15347 | HD23 | LEU | A1253 | 129.961 | 164.059 | 194.612 | 1.00 | 24.50 | H |
| ATOM | 15348 | N | ILE | A1254 | 131.130 | 166.837 | 194.180 | 1.00 | 22.27 | N |
| ATOM | 15349 | CA | ILE | A1254 | 132.383 | 166.340 | 194.740 | 1.00 | 22.27 | C |
| ATOM | 15350 | C | ILE | A1254 | 132.877 | 167.247 | 195.860 | 1.00 | 22.27 | C |
| ATOM | 15351 | O | ILE | A1254 | 133.372 | 166.770 | 196.888 | 1.00 | 22.27 | O |
| ATOM | 15352 | CB | ILE | A1254 | 133.438 | 166.196 | 193.627 | 1.00 | 22.27 | C |
| ATOM | 15353 | CG1 | ILE | A1254 | 133.099 | 165.003 | 192.729 | 1.00 | 22.27 | C |
| ATOM | 15354 | CG2 | ILE | A1254 | 134.834 | 166.045 | 194.232 | 1.00 | 22.27 | C |
| ATOM | 15355 | CD1 | ILE | A1254 | 133.997 | 164.866 | 191.515 | 1.00 | 22.27 | C |
| ATOM | 15356 | H | ILE | A1254 | 131.206 | 167.194 | 193.402 | 1.00 | 22.27 | H |
| ATOM | 15357 | HA | ILE | A1254 | 132.230 | 165.460 | 195.118 | 1.00 | 22.27 | H |
| ATOM | 15358 | HB | ILE | A1254 | 133.424 | 167.000 | 193.086 | 1.00 | 22.27 | H |
| ATOM | 15359 | HG12 | ILE | A1254 | 133.182 | 164.188 | 193.250 | 1.00 | 22.27 | H |
| ATOM | 15360 | HG13 | ILE | A1254 | 132.187 | 165.098 | 192.414 | 1.00 | 22.27 | H |
| ATOM | 15361 | HG21 | ILE | A1254 | 135.443 | 165.710 | 193.558 | 1.00 | 22.27 | H |
| ATOM | 15362 | HG22 | ILE | A1254 | 135.141 | 166.911 | 194.544 | 1.00 | 22.27 | H |
| ATOM | 15363 | HG23 | ILE | A1254 | 134.790 | 165.422 | 194.974 | 1.00 | 22.27 | H |
| ATOM | 15364 | HD11 | ILE | A1254 | 133.567 | 164.285 | 190.869 | 1.00 | 22.27 | H |
| ATOM | 15365 | HD12 | ILE | A1254 | 134.139 | 165.744 | 191.128 | 1.00 | 22.27 | H |
| ATOM | 15366 | HD13 | ILE | A1254 | 134.846 | 164.485 | 191.789 | 1.00 | 22.27 | H |
| ATOM | 15367 | N | GLU | A1255 | 132.760 | 168.565 | 195.681 | 1.00 | 25.31 | N |
| ATOM | 15368 | CA | GLU | A1255 | 133.384 | 169.498 | 196.615 | 1.00 | 25.31 | C |
| ATOM | 15369 | C | GLU | A1255 | 132.867 | 169.292 | 198.034 | 1.00 | 25.31 | C |
| ATOM | 15370 | O | GLU | A1255 | 133.646 | 169.275 | 198.995 | 1.00 | 25.31 | O |
| ATOM | 15371 | CB | GLU | A1255 | 133.137 | 170.936 | 196.158 | 1.00 | 25.31 | C |
| ATOM | 15372 | CG | GLU | A1255 | 133.947 | 171.346 | 194.938 | 1.00 | 25.31 | C |
| ATOM | 15373 | CD | GLU | A1255 | 133.468 | 172.649 | 194.327 | 1.00 | 25.31 | C |
| ATOM | 15374 | OE1 | GLU | A1255 | 132.363 | 173.107 | 194.686 | 1.00 | 25.31 | O |
| ATOM | 15375 | OE2 | GLU | A1255 | 134.199 | 173.219 | 193.490 | 1.00 | 25.31 | O |
| ATOM | 15376 | H | GLU | A1255 | 132.331 | 168.937 | 195.036 | 1.00 | 25.31 | H |
| ATOM | 15377 | HA | GLU | A1255 | 134.342 | 169.345 | 196.620 | 1.00 | 25.31 | H |
| ATOM | 15378 | HB2 | GLU | A1255 | 132.197 | 171.033 | 195.937 | 1.00 | 25.31 | H |
| ATOM | 15379 | HB3 | GLU | A1255 | 133.368 | 171.538 | 196.882 | 1.00 | 25.31 | H |
| ATOM | 15380 | HG2 | GLU | A1255 | 134.874 | 171.461 | 195.199 | 1.00 | 25.31 | H |
| ATOM | 15381 | HG3 | GLU | A1255 | 133.877 | 170.653 | 194.262 | 1.00 | 25.31 | H |
| ATOM | 15382 | N | THR | A1256 | 131.552 | 169.132 | 198.188 | 1.00 | 32.68 | N |
| ATOM | 15383 | CA | THR | A1256 | 130.980 | 169.003 | 199.525 | 1.00 | 32.68 | C |
| ATOM | 15384 | C | THR | A1256 | 131.323 | 167.657 | 200.151 | 1.00 | 32.68 | C |
| ATOM | 15385 | O | THR | A1256 | 131.572 | 167.575 | 201.360 | 1.00 | 32.68 | O |
| ATOM | 15386 | CB | THR | A1256 | 129.465 | 169.195 | 199.469 | 1.00 | 32.68 | C |
| ATOM | 15387 | OG1 | THR | A1256 | 128.891 | 168.238 | 198.569 | 1.00 | 32.68 | O |
| ATOM | 15388 | CG2 | THR | A1256 | 129.122 | 170.601 | 199.000 | 1.00 | 32.68 | C |
| ATOM | 15389 | H | THR | A1256 | 130.980 | 169.096 | 197.547 | 1.00 | 32.68 | H |
| ATOM | 15390 | HA | THR | A1256 | 131.349 | 169.699 | 200.092 | 1.00 | 32.68 | H |
| ATOM | 15391 | HB | THR | A1256 | 129.092 | 169.068 | 200.355 | 1.00 | 32.68 | H |
| ATOM | 15392 | HG1 | THR | A1256 | 128.059 | 168.347 | 198.527 | 1.00 | 32.68 | H |
| ATOM | 15393 | HG21 | THR | A1256 | 128.165 | 170.745 | 199.053 | 1.00 | 32.68 | H |
| ATOM | 15394 | HG22 | THR | A1256 | 129.570 | 171.255 | 199.559 | 1.00 | 32.68 | H |
| ATOM | 15395 | HG23 | THR | A1256 | 129.410 | 170.724 | 198.081 | 1.00 | 32.68 | H |
| ATOM | 15396 | N | TYR | A1257 | 131.342 | 166.593 | 199.353 | 1.00 | 33.33 | N |
| ATOM | 15397 | CA | TYR | A1257 | 131.603 | 165.250 | 199.856 | 1.00 | 33.33 | C |
| ATOM | 15398 | C | TYR | A1257 | 133.086 | 164.897 | 199.872 | 1.00 | 33.33 | C |
| ATOM | 15399 | O | TYR | A1257 | 133.438 | 163.800 | 200.318 | 1.00 | 33.33 | O |
| ATOM | 15400 | CB | TYR | A1257 | 130.823 | 164.225 | 199.027 | 1.00 | 33.33 | C |
| ATOM | 15401 | CG | TYR | A1257 | 129.323 | 164.333 | 199.209 | 1.00 | 33.33 | C |
| ATOM | 15402 | CD1 | TYR | A1257 | 128.664 | 163.578 | 200.170 | 1.00 | 33.33 | C |
| ATOM | 15403 | CD2 | TYR | A1257 | 128.572 | 165.207 | 198.436 | 1.00 | 33.33 | C |
| ATOM | 15404 | CE1 | TYR | A1257 | 127.295 | 163.680 | 200.343 | 1.00 | 33.33 | C |
| ATOM | 15405 | CE2 | TYR | A1257 | 127.205 | 165.316 | 198.603 | 1.00 | 33.33 | C |
| ATOM | 15406 | CZ | TYR | A1257 | 126.572 | 164.551 | 199.557 | 1.00 | 33.33 | C |
| ATOM | 15407 | OH | TYR | A1257 | 125.211 | 164.660 | 199.724 | 1.00 | 33.33 | O |
| ATOM | 15408 | H | TYR | A1257 | 131.203 | 166.624 | 198.504 | 1.00 | 33.33 | H |
| ATOM | 15409 | HA | TYR | A1257 | 131.279 | 165.195 | 200.768 | 1.00 | 33.33 | H |
| ATOM | 15410 | HB2 | TYR | A1257 | 131.020 | 164.363 | 198.087 | 1.00 | 33.33 | H |
| ATOM | 15411 | HB3 | TYR | A1257 | 131.092 | 163.333 | 199.294 | 1.00 | 33.33 | H |
| ATOM | 15412 | HD1 | TYR | A1257 | 129.149 | 162.989 | 200.700 | 1.00 | 33.33 | H |
| ATOM | 15413 | HD2 | TYR | A1257 | 128.993 | 165.724 | 197.793 | 1.00 | 33.33 | H |
| ATOM | 15414 | HE1 | TYR | A1257 | 126.866 | 163.165 | 200.987 | 1.00 | 33.33 | H |
| ATOM | 15415 | HE2 | TYR | A1257 | 126.716 | 165.904 | 198.074 | 1.00 | 33.33 | H |
| ATOM | 15416 | HH | TYR | A1257 | 124.960 | 164.183 | 200.368 | 1.00 | 33.33 | H |
| ATOM | 15417 | N | PHE | A1258 | 133.953 | 165.791 | 199.402 | 1.00 | 32.31 | N |
| ATOM | 15418 | CA | PHE | A1258 | 135.399 | 165.689 | 199.602 | 1.00 | 32.31 | C |
| ATOM | 15419 | C | PHE | A1258 | 135.929 | 164.303 | 199.232 | 1.00 | 32.31 | C |
| ATOM | 15420 | O | PHE | A1258 | 136.403 | 163.540 | 200.075 | 1.00 | 32.31 | O |
| ATOM | 15421 | CB | PHE | A1258 | 135.768 | 166.048 | 201.044 | 1.00 | 32.31 | C |
| ATOM | 15422 | N | VAL | A1259 | 135.833 | 163.980 | 197.941 | 1.00 | 28.46 | N |
| ATOM | 15423 | CA | VAL | A1259 | 136.362 | 162.705 | 197.459 | 1.00 | 28.46 | C |
| ATOM | 15424 | C | VAL | A1259 | 137.874 | 162.656 | 197.652 | 1.00 | 28.46 | C |
| ATOM | 15425 | O | VAL | A1259 | 138.397 | 161.825 | 198.403 | 1.00 | 28.46 | O |
| ATOM | 15426 | CB | VAL | A1259 | 135.974 | 162.481 | 195.987 | 1.00 | 28.46 | C |
| ATOM | 15427 | N | SER | A1260 | 138.597 | 163.555 | 196.981 | 1.00 | 19.09 | N |
| ATOM | 15428 | CA | SER | A1260 | 140.041 | 163.666 | 197.151 | 1.00 | 19.09 | C |
| ATOM | 15429 | C | SER | A1260 | 140.562 | 164.900 | 196.420 | 1.00 | 19.09 | C |
| ATOM | 15430 | O | SER | A1260 | 140.083 | 165.214 | 195.323 | 1.00 | 19.09 | O |
| ATOM | 15431 | CB | SER | A1260 | 140.747 | 162.410 | 196.635 | 1.00 | 19.09 | C |
| ATOM | 15432 | OG | SER | A1260 | 142.154 | 162.531 | 196.759 | 1.00 | 19.09 | O |
| ATOM | 15433 | HA | SER | A1260 | 140.234 | 163.749 | 198.097 | 1.00 | 19.09 | H |
| ATOM | 15434 | HB2 | SER | A1260 | 140.452 | 161.644 | 197.151 | 1.00 | 19.09 | H |
| ATOM | 15435 | HB3 | SER | A1260 | 140.524 | 162.285 | 195.699 | 1.00 | 19.09 | H |
| ATOM | 15436 | HG | SER | A1260 | 142.362 | 162.647 | 197.565 | 1.00 | 19.09 | H |
| ATOM | 15437 | N | PRO | A1261 | 141.534 | 165.626 | 196.985 | 1.00 | 14.96 | N |
| ATOM | 15438 | CA | PRO | A1261 | 142.071 | 166.793 | 196.262 | 1.00 | 14.96 | C |
| ATOM | 15439 | C | PRO | A1261 | 142.656 | 166.448 | 194.903 | 1.00 | 14.96 | C |
| ATOM | 15440 | O | PRO | A1261 | 142.472 | 167.208 | 193.943 | 1.00 | 14.96 | O |
| ATOM | 15441 | CB | PRO | A1261 | 143.145 | 167.339 | 197.214 | 1.00 | 14.96 | C |
| ATOM | 15442 | CG | PRO | A1261 | 142.810 | 166.799 | 198.554 | 1.00 | 14.96 | C |
| ATOM | 15443 | CD | PRO | A1261 | 142.066 | 165.518 | 198.355 | 1.00 | 14.96 | C |
| ATOM | 15444 | HA | PRO | A1261 | 141.377 | 167.462 | 196.148 | 1.00 | 14.96 | H |
| ATOM | 15445 | HB2 | PRO | A1261 | 144.018 | 167.028 | 196.930 | 1.00 | 14.96 | H |
| ATOM | 15446 | HB3 | PRO | A1261 | 143.112 | 168.308 | 197.219 | 1.00 | 14.96 | H |
| ATOM | 15447 | HG2 | PRO | A1261 | 143.630 | 166.637 | 199.047 | 1.00 | 14.96 | H |
| ATOM | 15448 | HG3 | PRO | A1261 | 142.255 | 167.439 | 199.026 | 1.00 | 14.96 | H |
| ATOM | 15449 | HD2 | PRO | A1261 | 142.670 | 164.762 | 198.425 | 1.00 | 14.96 | H |
| ATOM | 15450 | HD3 | PRO | A1261 | 141.340 | 165.452 | 198.996 | 1.00 | 14.96 | H |
| ATOM | 15451 | N | THR | A1262 | 143.357 | 165.318 | 194.791 | 1.00 | 10.97 | N |
| ATOM | 15452 | CA | THR | A1262 | 143.983 | 164.964 | 193.520 | 1.00 | 10.97 | C |
| ATOM | 15453 | C | THR | A1262 | 142.936 | 164.634 | 192.464 | 1.00 | 10.97 | C |
| ATOM | 15454 | O | THR | A1262 | 143.101 | 164.982 | 191.289 | 1.00 | 10.97 | O |
| ATOM | 15455 | CB | THR | A1262 | 144.938 | 163.786 | 193.710 | 1.00 | 10.97 | C |
| ATOM | 15456 | OG1 | THR | A1262 | 145.852 | 164.076 | 194.775 | 1.00 | 10.97 | O |
| ATOM | 15457 | CG2 | THR | A1262 | 145.723 | 163.519 | 192.431 | 1.00 | 10.97 | C |
| ATOM | 15458 | H | THR | A1262 | 143.483 | 164.749 | 195.424 | 1.00 | 10.97 | H |
| ATOM | 15459 | HA | THR | A1262 | 144.500 | 165.721 | 193.202 | 1.00 | 10.97 | H |
| ATOM | 15460 | HB | THR | A1262 | 144.428 | 162.991 | 193.928 | 1.00 | 10.97 | H |
| ATOM | 15461 | HG1 | THR | A1262 | 146.413 | 163.456 | 194.846 | 1.00 | 10.97 | H |
| ATOM | 15462 | HG21 | THR | A1262 | 146.420 | 162.868 | 192.602 | 1.00 | 10.97 | H |
| ATOM | 15463 | HG22 | THR | A1262 | 145.132 | 163.175 | 191.743 | 1.00 | 10.97 | H |
| ATOM | 15464 | HG23 | THR | A1262 | 146.131 | 164.341 | 192.116 | 1.00 | 10.97 | H |
| ATOM | 15465 | N | LEU | A1263 | 141.857 | 163.958 | 192.860 | 1.00 | 11.85 | N |
| ATOM | 15466 | CA | LEU | A1263 | 140.784 | 163.671 | 191.914 | 1.00 | 11.85 | C |
| ATOM | 15467 | C | LEU | A1263 | 140.143 | 164.959 | 191.415 | 1.00 | 11.85 | C |
| ATOM | 15468 | O | LEU | A1263 | 139.828 | 165.086 | 190.225 | 1.00 | 11.85 | O |
| ATOM | 15469 | CB | LEU | A1263 | 139.743 | 162.762 | 192.570 | 1.00 | 11.85 | C |
| ATOM | 15470 | CG | LEU | A1263 | 138.428 | 162.548 | 191.817 | 1.00 | 11.85 | C |
| ATOM | 15471 | CD1 | LEU | A1263 | 138.667 | 161.748 | 190.548 | 1.00 | 11.85 | C |
| ATOM | 15472 | CD2 | LEU | A1263 | 137.419 | 161.847 | 192.709 | 1.00 | 11.85 | C |
| ATOM | 15473 | H | LEU | A1263 | 141.725 | 163.660 | 193.656 | 1.00 | 11.85 | H |
| ATOM | 15474 | HA | LEU | A1263 | 141.153 | 163.202 | 191.149 | 1.00 | 11.85 | H |
| ATOM | 15475 | HB2 | LEU | A1263 | 140.144 | 161.888 | 192.697 | 1.00 | 11.85 | H |
| ATOM | 15476 | HB3 | LEU | A1263 | 139.518 | 163.138 | 193.435 | 1.00 | 11.85 | H |
| ATOM | 15477 | HG | LEU | A1263 | 138.053 | 163.406 | 191.566 | 1.00 | 11.85 | H |
| ATOM | 15478 | HD11 | LEU | A1263 | 137.822 | 161.624 | 190.089 | 1.00 | 11.85 | H |
| ATOM | 15479 | HD12 | LEU | A1263 | 139.284 | 162.236 | 189.980 | 1.00 | 11.85 | H |
| ATOM | 15480 | HD13 | LEU | A1263 | 139.045 | 160.887 | 190.785 | 1.00 | 11.85 | H |
| ATOM | 15481 | HD21 | LEU | A1263 | 136.594 | 161.721 | 192.215 | 1.00 | 11.85 | H |
| ATOM | 15482 | HD22 | LEU | A1263 | 137.779 | 160.988 | 192.978 | 1.00 | 11.85 | H |
| ATOM | 15483 | HD23 | LEU | A1263 | 137.255 | 162.397 | 193.492 | 1.00 | 11.85 | H |
| ATOM | 15484 | N | PHE | A1264 | 139.935 | 165.925 | 192.313 | 1.00 | 9.59 | N |
| ATOM | 15485 | CA | PHE | A1264 | 139.396 | 167.216 | 191.898 | 1.00 | 9.59 | C |
| ATOM | 15486 | C | PHE | A1264 | 140.348 | 167.915 | 190.936 | 1.00 | 9.59 | C |
| ATOM | 15487 | O | PHE | A1264 | 139.920 | 168.474 | 189.919 | 1.00 | 9.59 | O |
| ATOM | 15488 | CB | PHE | A1264 | 139.132 | 168.086 | 193.127 | 1.00 | 9.59 | C |
| ATOM | 15489 | CG | PHE | A1264 | 138.433 | 169.380 | 192.821 | 1.00 | 9.59 | C |
| ATOM | 15490 | CD1 | PHE | A1264 | 137.241 | 169.391 | 192.115 | 1.00 | 9.59 | C |
| ATOM | 15491 | CD2 | PHE | A1264 | 138.965 | 170.586 | 193.246 | 1.00 | 9.59 | C |
| ATOM | 15492 | CE1 | PHE | A1264 | 136.595 | 170.581 | 191.837 | 1.00 | 9.59 | C |
| ATOM | 15493 | CE2 | PHE | A1264 | 138.324 | 171.779 | 192.969 | 1.00 | 9.59 | C |
| ATOM | 15494 | CZ | PHE | A1264 | 137.138 | 171.776 | 192.264 | 1.00 | 9.59 | C |
| ATOM | 15495 | H | PHE | A1264 | 140.095 | 165.857 | 193.155 | 1.00 | 9.59 | H |
| ATOM | 15496 | HA | PHE | A1264 | 138.554 | 167.072 | 191.438 | 1.00 | 9.59 | H |
| ATOM | 15497 | HB2 | PHE | A1264 | 138.576 | 167.589 | 193.748 | 1.00 | 9.59 | H |
| ATOM | 15498 | HB3 | PHE | A1264 | 139.980 | 168.300 | 193.546 | 1.00 | 9.59 | H |
| ATOM | 15499 | HD1 | PHE | A1264 | 136.871 | 168.589 | 191.825 | 1.00 | 9.59 | H |
| ATOM | 15500 | HD2 | PHE | A1264 | 139.764 | 170.593 | 193.722 | 1.00 | 9.59 | H |
| ATOM | 15501 | HE1 | PHE | A1264 | 135.797 | 170.577 | 191.361 | 1.00 | 9.59 | H |
| ATOM | 15502 | HE2 | PHE | A1264 | 138.692 | 172.582 | 193.259 | 1.00 | 9.59 | H |
| ATOM | 15503 | HZ | PHE | A1264 | 136.705 | 172.578 | 192.077 | 1.00 | 9.59 | H |
| ATOM | 15504 | N | ARG | A1265 | 141.650 | 167.876 | 191.231 | 1.00 | 7.07 | N |
| ATOM | 15505 | CA | ARG | A1265 | 142.630 | 168.475 | 190.332 | 1.00 | 7.07 | C |
| ATOM | 15506 | C | ARG | A1265 | 142.580 | 167.824 | 188.956 | 1.00 | 7.07 | C |
| ATOM | 15507 | O | ARG | A1265 | 142.653 | 168.511 | 187.931 | 1.00 | 7.07 | O |
| ATOM | 15508 | CB | ARG | A1265 | 144.031 | 168.344 | 190.929 | 1.00 | 7.07 | C |
| ATOM | 15509 | CG | ARG | A1265 | 144.303 | 169.258 | 192.112 | 1.00 | 7.07 | C |
| ATOM | 15510 | CD | ARG | A1265 | 145.646 | 168.935 | 192.754 | 1.00 | 7.07 | C |
| ATOM | 15511 | NE | ARG | A1265 | 146.603 | 170.030 | 192.630 | 1.00 | 7.07 | N |
| ATOM | 15512 | CZ | ARG | A1265 | 147.853 | 169.988 | 193.084 | 1.00 | 7.07 | C |
| ATOM | 15513 | NH1 | ARG | A1265 | 148.308 | 168.904 | 193.697 | 1.00 | 7.07 | N |
| ATOM | 15514 | NH2 | ARG | A1265 | 148.649 | 171.033 | 192.922 | 1.00 | 7.07 | N |
| ATOM | 15515 | H | ARG | A1265 | 141.984 | 167.514 | 191.936 | 1.00 | 7.07 | H |
| ATOM | 15516 | HA | ARG | A1265 | 142.432 | 169.419 | 190.228 | 1.00 | 7.07 | H |
| ATOM | 15517 | HB2 | ARG | A1265 | 144.155 | 167.430 | 191.228 | 1.00 | 7.07 | H |
| ATOM | 15518 | HB3 | ARG | A1265 | 144.682 | 168.553 | 190.240 | 1.00 | 7.07 | H |
| ATOM | 15519 | HG2 | ARG | A1265 | 144.323 | 170.179 | 191.809 | 1.00 | 7.07 | H |
| ATOM | 15520 | HG3 | ARG | A1265 | 143.609 | 169.137 | 192.779 | 1.00 | 7.07 | H |
| ATOM | 15521 | HD2 | ARG | A1265 | 145.512 | 168.759 | 193.698 | 1.00 | 7.07 | H |
| ATOM | 15522 | HD3 | ARG | A1265 | 146.026 | 168.154 | 192.321 | 1.00 | 7.07 | H |
| ATOM | 15523 | HE | ARG | A1265 | 146.325 | 170.769 | 192.290 | 1.00 | 7.07 | H |
| ATOM | 15524 | HH11 | ARG | A1265 | 147.797 | 168.221 | 193.806 | 1.00 | 7.07 | H |
| ATOM | 15525 | HH12 | ARG | A1265 | 149.117 | 168.885 | 193.989 | 1.00 | 7.07 | H |
| ATOM | 15526 | HH21 | ARG | A1265 | 148.356 | 171.736 | 192.524 | 1.00 | 7.07 | H |
| ATOM | 15527 | HH22 | ARG | A1265 | 149.458 | 171.009 | 193.215 | 1.00 | 7.07 | H |
| ATOM | 15528 | N | VAL | A1266 | 142.459 | 166.497 | 188.916 | 1.00 | 7.00 | N |
| ATOM | 15529 | CA | VAL | A1266 | 142.464 | 165.784 | 187.643 | 1.00 | 7.00 | C |
| ATOM | 15530 | C | VAL | A1266 | 141.209 | 166.110 | 186.842 | 1.00 | 7.00 | C |
| ATOM | 15531 | O | VAL | A1266 | 141.279 | 166.408 | 185.644 | 1.00 | 7.00 | O |
| ATOM | 15532 | CB | VAL | A1266 | 142.602 | 164.270 | 187.880 | 1.00 | 7.00 | C |
| ATOM | 15533 | CG1 | VAL | A1266 | 142.487 | 163.512 | 186.565 | 1.00 | 7.00 | C |
| ATOM | 15534 | CG2 | VAL | A1266 | 143.922 | 163.963 | 188.568 | 1.00 | 7.00 | C |
| ATOM | 15535 | H | VAL | A1266 | 142.375 | 165.992 | 189.607 | 1.00 | 7.00 | H |
| ATOM | 15536 | HA | VAL | A1266 | 143.231 | 166.074 | 187.124 | 1.00 | 7.00 | H |
| ATOM | 15537 | N | ILE | A1267 | 140.040 | 166.058 | 187.488 | 1.00 | 8.29 | N |
| ATOM | 15538 | CA | ILE | A1267 | 138.799 | 166.319 | 186.768 | 1.00 | 8.29 | C |
| ATOM | 15539 | C | ILE | A1267 | 138.662 | 167.790 | 186.400 | 1.00 | 8.29 | C |
| ATOM | 15540 | O | ILE | A1267 | 137.869 | 168.128 | 185.514 | 1.00 | 8.29 | O |
| ATOM | 15541 | CB | ILE | A1267 | 137.578 | 165.848 | 187.583 | 1.00 | 8.29 | C |
| ATOM | 15542 | CG1 | ILE | A1267 | 137.429 | 166.673 | 188.862 | 1.00 | 8.29 | C |
| ATOM | 15543 | CG2 | ILE | A1267 | 137.691 | 164.362 | 187.902 | 1.00 | 8.29 | C |
| ATOM | 15544 | CD1 | ILE | A1267 | 136.359 | 167.740 | 188.775 | 1.00 | 8.29 | C |
| ATOM | 15545 | H | ILE | A1267 | 139.943 | 165.878 | 188.323 | 1.00 | 8.29 | H |
| ATOM | 15546 | HA | ILE | A1267 | 138.810 | 165.811 | 185.942 | 1.00 | 8.29 | H |
| ATOM | 15547 | HB | ILE | A1267 | 136.784 | 165.982 | 187.042 | 1.00 | 8.29 | H |
| ATOM | 15548 | HG12 | ILE | A1267 | 137.196 | 166.079 | 189.592 | 1.00 | 8.29 | H |
| ATOM | 15549 | HG13 | ILE | A1267 | 138.270 | 167.111 | 189.051 | 1.00 | 8.29 | H |
| ATOM | 15550 | HG21 | ILE | A1267 | 136.908 | 164.087 | 188.402 | 1.00 | 8.29 | H |
| ATOM | 15551 | HG22 | ILE | A1267 | 137.743 | 163.866 | 187.070 | 1.00 | 8.29 | H |
| ATOM | 15552 | HG23 | ILE | A1267 | 138.491 | 164.209 | 188.428 | 1.00 | 8.29 | H |
| ATOM | 15553 | HD11 | ILE | A1267 | 136.367 | 168.261 | 189.594 | 1.00 | 8.29 | H |
| ATOM | 15554 | HD12 | ILE | A1267 | 136.544 | 168.315 | 188.016 | 1.00 | 8.29 | H |
| ATOM | 15555 | HD13 | ILE | A1267 | 135.496 | 167.313 | 188.664 | 1.00 | 8.29 | H |
| ATOM | 15556 | N | ARG | A1268 | 139.412 | 168.681 | 187.053 | 1.00 | 6.16 | N |
| ATOM | 15557 | CA | ARG | A1268 | 139.418 | 170.081 | 186.649 | 1.00 | 6.16 | C |
| ATOM | 15558 | C | ARG | A1268 | 140.088 | 170.302 | 185.299 | 1.00 | 6.16 | C |
| ATOM | 15559 | O | ARG | A1268 | 139.953 | 171.392 | 184.733 | 1.00 | 6.16 | O |
| ATOM | 15560 | CB | ARG | A1268 | 140.121 | 170.928 | 187.710 | 1.00 | 6.16 | C |
| ATOM | 15561 | CG | ARG | A1268 | 139.188 | 171.525 | 188.751 | 1.00 | 6.16 | C |
| ATOM | 15562 | CD | ARG | A1268 | 139.933 | 172.472 | 189.680 | 1.00 | 6.16 | C |
| ATOM | 15563 | NE | ARG | A1268 | 140.280 | 173.728 | 189.018 | 1.00 | 6.16 | N |
| ATOM | 15564 | CZ | ARG | A1268 | 141.330 | 174.484 | 189.330 | 1.00 | 6.16 | C |
| ATOM | 15565 | NH1 | ARG | A1268 | 142.157 | 174.126 | 190.304 | 1.00 | 6.16 | N |
| ATOM | 15566 | NH2 | ARG | A1268 | 141.554 | 175.609 | 188.664 | 1.00 | 6.16 | N |
| ATOM | 15567 | H | ARG | A1268 | 139.918 | 168.499 | 187.724 | 1.00 | 6.16 | H |
| ATOM | 15568 | HA | ARG | A1268 | 138.501 | 170.390 | 186.580 | 1.00 | 6.16 | H |
| ATOM | 15569 | HB2 | ARG | A1268 | 140.767 | 170.374 | 188.176 | 1.00 | 6.16 | H |
| ATOM | 15570 | HB3 | ARG | A1268 | 140.578 | 171.662 | 187.270 | 1.00 | 6.16 | H |
| ATOM | 15571 | HG2 | ARG | A1268 | 138.487 | 172.025 | 188.305 | 1.00 | 6.16 | H |
| ATOM | 15572 | HG3 | ARG | A1268 | 138.806 | 170.812 | 189.286 | 1.00 | 6.16 | H |
| ATOM | 15573 | HD2 | ARG | A1268 | 139.372 | 172.677 | 190.444 | 1.00 | 6.16 | H |
| ATOM | 15574 | HD3 | ARG | A1268 | 140.751 | 172.041 | 189.971 | 1.00 | 6.16 | H |
| ATOM | 15575 | HE | ARG | A1268 | 139.755 | 174.011 | 188.397 | 1.00 | 6.16 | H |
| ATOM | 15576 | HH11 | ARG | A1268 | 142.023 | 173.401 | 190.744 | 1.00 | 6.16 | H |
| ATOM | 557 | HH12 | ARG | A1268 | 142.831 | 174.624 | 190.496 | 1.00 | 6.16 | H |
| ATOM | 15578 | HH21 | ARG | A1268 | 141.022 | 175.847 | 188.031 | 1.00 | 6.16 | H |
| ATOM | 15579 | HH22 | ARG | A1268 | 142.231 | 176.099 | 188.863 | 1.00 | 6.16 | H |
| ATOM | 15580 | N | LEU | A1269 | 140.801 | 169.304 | 184.770 | 1.00 | 7.10 | N |
| ATOM | 15581 | CA | LEU | A1269 | 141.495 | 169.477 | 183.499 | 1.00 | 7.10 | C |
| ATOM | 15582 | C | LEU | A1269 | 140.538 | 169.753 | 182.348 | 1.00 | 7.10 | C |
| ATOM | 15583 | O | LEU | A1269 | 140.958 | 170.305 | 181.326 | 1.00 | 7.10 | O |
| ATOM | 15584 | CB | LEU | A1269 | 142.335 | 168.237 | 183.187 | 1.00 | 7.10 | C |
| ATOM | 15585 | CG | LEU | A1269 | 143.619 | 168.075 | 184.005 | 1.00 | 7.10 | C |
| ATOM | 15586 | CD1 | LEU | A1269 | 144.122 | 166.641 | 183.934 | 1.00 | 7.10 | C |
| ATOM | 15587 | CD2 | LEU | A1269 | 144.691 | 169.043 | 183.524 | 1.00 | 7.10 | C |
| ATOM | 15588 | H | LEU | A1269 | 140.896 | 168.526 | 185.125 | 1.00 | 7.10 | H |
| ATOM | 15589 | HA | LEU | A1269 | 142.096 | 170.235 | 183.573 | 1.00 | 7.10 | H |
| ATOM | 15590 | HB2 | LEU | A1269 | 141.790 | 167.451 | 183.346 | 1.00 | 7.10 | H |
| ATOM | 15591 | HB3 | LEU | A1269 | 142.590 | 168.269 | 182.252 | 1.00 | 7.10 | H |
| ATOM | 15592 | HG | LEU | A1269 | 143.428 | 168.279 | 184.934 | 1.00 | 7.10 | H |
| ATOM | 15593 | HD11 | LEU | A1269 | 143.708 | 166.128 | 184.646 | 1.00 | 7.10 | H |
| ATOM | 15594 | HD12 | LEU | A1269 | 143.884 | 166.265 | 183.073 | 1.00 | 7.10 | H |
| ATOM | 15595 | HD13 | LEU | A1269 | 145.086 | 166.639 | 184.044 | 1.00 | 7.10 | H |
| ATOM | 15596 | HD21 | LEU | A1269 | 145.459 | 168.979 | 184.113 | 1.00 | 7.10 | H |
| ATOM | 15597 | HD22 | LEU | A1269 | 144.947 | 168.805 | 182.619 | 1.00 | 7.10 | H |
| ATOM | 15598 | HD23 | LEU | A1269 | 144.335 | 169.945 | 183.543 | 1.00 | 7.10 | H |
| ATOM | 15599 | N | ALA | A1270 | 139.263 | 169.381 | 182.486 | 1.00 | 4.99 | N |
| ATOM | 15600 | CA | ALA | A1270 | 138.297 | 169.642 | 181.426 | 1.00 | 4.99 | C |
| ATOM | 15601 | C | ALA | A1270 | 138.042 | 171.131 | 181.231 | 1.00 | 4.99 | C |
| ATOM | 15602 | O | ALA | A1270 | 137.486 | 171.523 | 180.199 | 1.00 | 4.99 | O |
| ATOM | 15603 | CB | ALA | A1270 | 136.984 | 168.923 | 181.729 | 1.00 | 4.99 | C |
| ATOM | 15604 | H | ALA | A1270 | 138.939 | 168.980 | 183.174 | 1.00 | 4.99 | H |
| ATOM | 15605 | HA | ALA | A1270 | 138.644 | 169.289 | 180.592 | 1.00 | 4.99 | H |
| ATOM | 15606 | HB1 | ALA | A1270 | 136.313 | 169.202 | 181.087 | 1.00 | 4.99 | H |
| ATOM | 15607 | HB2 | ALA | A1270 | 137.126 | 167.965 | 181.668 | 1.00 | 4.99 | H |
| ATOM | 15608 | HB3 | ALA | A1270 | 136.706 | 169.160 | 182.625 | 1.00 | 4.99 | H |
| ATOM | 15609 | N | ARG | A1271 | 138.430 | 171.970 | 182.196 | 1.00 | 4.81 | N |
| ATOM | 15610 | CA | ARG | A1271 | 138.264 | 173.410 | 182.041 | 1.00 | 4.81 | C |
| ATOM | 15611 | C | ARG | A1271 | 139.013 | 173.945 | 180.828 | 1.00 | 4.81 | C |
| ATOM | 15612 | O | ARG | A1271 | 138.627 | 174.985 | 180.283 | 1.00 | 4.81 | O |
| ATOM | 15613 | CB | ARG | A1271 | 138.739 | 174.131 | 183.306 | 1.00 | 4.81 | C |
| ATOM | 15614 | CG | ARG | A1271 | 140.254 | 174.255 | 183.425 | 1.00 | 4.81 | C |
| ATOM | 15615 | CD | ARG | A1271 | 140.669 | 174.878 | 184.747 | 1.00 | 4.81 | C |
| ATOM | 15616 | NE | ARG | A1271 | 142.125 | 174.931 | 184.881 | 1.00 | 4.81 | N |
| ATOM | 15617 | CZ | ARG | A1271 | 142.818 | 175.982 | 185.312 | 1.00 | 4.81 | C |
| ATOM | 15618 | NH1 | ARG | A1271 | 142.208 | 177.105 | 185.673 | 1.00 | 4.81 | N |
| ATOM | 15619 | NH2 | ARG | A1271 | 144.140 | 175.908 | 185.386 | 1.00 | 4.81 | N |
| ATOM | 15620 | H | ARG | A1271 | 138.787 | 171.730 | 182.941 | 1.00 | 4.81 | H |
| ATOM | 15621 | HA | ARG | A1271 | 137.322 | 173.607 | 181.921 | 1.00 | 4.81 | H |
| ATOM | 15622 | HB2 | ARG | A1271 | 138.369 | 175.028 | 183.310 | 1.00 | 4.81 | H |
| ATOM | 15623 | HB3 | ARG | A1271 | 138.420 | 173.643 | 184.081 | 1.00 | 4.81 | H |
| ATOM | 15624 | HG2 | ARG | A1271 | 140.652 | 173.372 | 183.367 | 1.00 | 4.81 | H |
| ATOM | 15625 | HG3 | ARG | A1271 | 140.591 | 174.822 | 182.715 | 1.00 | 4.81 | H |
| ATOM | 15626 | HD2 | ARG | A1271 | 140.309 | 175.776 | 184.790 | 1.00 | 4.81 | H |
| ATOM | 15627 | HD3 | ARG | A1271 | 140.317 | 174.343 | 185.476 | 1.00 | 4.81 | H |
| ATOM | 15628 | HE | ARG | A1271 | 142.568 | 174.227 | 184.661 | 1.00 | 4.81 | H |
| ATOM | 15629 | HH11 | ARG | A1271 | 141.354 | 177.173 | 185.632 | 1.00 | 4.81 | H |
| ATOM | 15630 | HH12 | ARG | A1271 | 142.675 | 177.773 | 185.950 | 1.00 | 4.81 | H |
| ATOM | 15631 | HH21 | ARG | A1271 | 144.543 | 175.184 | 185.156 | 1.00 | 4.81 | H |
| ATOM | 15632 | HH22 | ARG | A1271 | 144.594 | 176.583 | 185.666 | 1.00 | 4.81 | H |
| ATOM | 15633 | N | ILE | A1272 | 140.071 | 173.258 | 180.393 | 1.00 | 4.75 | N |
| ATOM | 15634 | CA | ILE | A1272 | 140.836 | 173.705 | 179.236 | 1.00 | 4.75 | C |
| ATOM | 15635 | C | ILE | A1272 | 140.004 | 173.665 | 177.961 | 1.00 | 4.75 | C |
| ATOM | 15636 | O | ILE | A1272 | 140.358 | 174.321 | 176.974 | 1.00 | 4.75 | O |
| ATOM | 15637 | CB | ILE | A1272 | 142.107 | 172.846 | 179.077 | 1.00 | 4.75 | C |
| ATOM | 15638 | CG1 | ILE | A1272 | 142.950 | 172.879 | 180.357 | 1.00 | 4.75 | C |
| ATOM | 15639 | CG2 | ILE | A1272 | 142.932 | 173.319 | 177.889 | 1.00 | 4.75 | C |
| ATOM | 15640 | CD1 | ILE | A1272 | 143.417 | 174.264 | 180.765 | 1.00 | 4.75 | C |
| ATOM | 15641 | H | ILE | A1272 | 140.361 | 172.531 | 180.750 | 1.00 | 4.75 | H |
| ATOM | 15642 | HA | ILE | A1272 | 141.110 | 174.624 | 179.377 | 1.00 | 4.75 | H |
| ATOM | 15643 | HB | ILE | A1272 | 141.836 | 171.930 | 178.916 | 1.00 | 4.75 | H |
| ATOM | 15644 | HG12 | ILE | A1272 | 142.427 | 172.516 | 181.089 | 1.00 | 4.75 | H |
| ATOM | 15645 | HG13 | ILE | A1272 | 143.740 | 172.332 | 180.221 | 1.00 | 4.75 | H |
| ATOM | 15646 | HG21 | ILE | A1272 | 143.818 | 172.929 | 177.949 | 1.00 | 4.75 | H |
| ATOM | 15647 | HG22 | ILE | A1272 | 142.501 | 173.032 | 177.068 | 1.00 | 4.75 | H |
| ATOM | 15648 | HG23 | ILE | A1272 | 142.995 | 174.286 | 177.912 | 1.00 | 4.75 | H |
| ATOM | 15649 | HD11 | ILE | A1272 | 144.010 | 174.182 | 181.529 | 1.00 | 4.75 | H |
| ATOM | 15650 | HD12 | ILE | A1272 | 143.891 | 174.672 | 180.024 | 1.00 | 4.75 | H |
| ATOM | 15651 | HD13 | ILE | A1272 | 142.647 | 174.803 | 181.003 | 1.00 | 4.75 | H |
| ATOM | 15652 | N | GLY | A1273 | 138.902 | 172.913 | 177.954 | 1.00 | 5.01 | N |
| ATOM | 15653 | CA | GLY | A1273 | 138.130 | 172.756 | 176.732 | 1.00 | 5.01 | C |
| ATOM | 15654 | C | GLY | A1273 | 137.619 | 174.068 | 176.171 | 1.00 | 5.01 | C |
| ATOM | 15655 | O | GLY | A1273 | 137.660 | 174.293 | 174.959 | 1.00 | 5.01 | O |
| ATOM | 15656 | H | GLY | A1273 | 138.589 | 172.489 | 178.634 | 1.00 | 5.01 | H |
| ATOM | 15657 | HA2 | GLY | A1273 | 138.681 | 172.330 | 176.057 | 1.00 | 5.01 | H |
| ATOM | 15658 | HA3 | GLY | A1273 | 137.367 | 172.183 | 176.908 | 1.00 | 5.01 | H |
| ATOM | 15659 | N | ARG | A1274 | 137.133 | 174.957 | 177.040 | 1.00 | 5.16 | N |
| ATOM | 15660 | CA | ARG | A1274 | 136.546 | 176.204 | 176.564 | 1.00 | 5.16 | C |
| ATOM | 15661 | C | ARG | A1274 | 137.592 | 177.252 | 176.206 | 1.00 | 5.16 | C |
| ATOM | 15662 | O | ARG | A1274 | 137.235 | 178.295 | 175.648 | 1.00 | 5.16 | O |
| ATOM | 15663 | CB | ARG | A1274 | 135.577 | 176.770 | 177.608 | 1.00 | 5.16 | C |
| ATOM | 15664 | CG | ARG | A1274 | 136.194 | 177.120 | 178.952 | 1.00 | 5.16 | C |
| ATOM | 15665 | CD | ARG | A1274 | 135.114 | 177.567 | 179.928 | 1.00 | 5.16 | C |
| ATOM | 15666 | NE | ARG | A1274 | 135.652 | 177.939 | 181.233 | 1.00 | 5.16 | N |
| ATOM | 15667 | CZ | ARG | A1274 | 135.921 | 177.077 | 182.210 | 1.00 | 5.16 | C |
| ATOM | 15668 | NH1 | ARG | A1274 | 135.701 | 175.779 | 182.043 | 1.00 | 5.16 | N |
| ATOM | 15669 | NH2 | ARG | A1274 | 136.408 | 177.516 | 183.362 | 1.00 | 5.16 | N |
| ATOM | 15670 | H | ARG | A1274 | 137.131 | 174.861 | 177.894 | 1.00 | 5.16 | H |
| ATOM | 15671 | HA | ARG | A1274 | 136.034 | 176.018 | 175.762 | 1.00 | 5.16 | H |
| ATOM | 15672 | HB2 | ARG | A1274 | 135.178 | 177.579 | 177.251 | 1.00 | 5.16 | H |
| ATOM | 15673 | HB3 | ARG | A1274 | 134.883 | 176.111 | 177.770 | 1.00 | 5.16 | H |
| ATOM | 15674 | HG2 | ARG | A1274 | 136.636 | 176.339 | 179.320 | 1.00 | 5.16 | H |
| ATOM | 15675 | HG3 | ARG | A1274 | 136.826 | 177.847 | 178.839 | 1.00 | 5.16 | H |
| ATOM | 15676 | HD2 | ARG | A1274 | 134.660 | 178.343 | 179.561 | 1.00 | 5.16 | H |
| ATOM | 15677 | HD3 | ARG | A1274 | 134.480 | 176.844 | 180.054 | 1.00 | 5.16 | H |
| ATOM | 15678 | HE | ARG | A1274 | 135.726 | 178.777 | 181.410 | 1.00 | 5.16 | H |
| ATOM | 15679 | HH11 | ARG | A1274 | 135.385 | 175.483 | 181.301 | 1.00 | 5.16 | H |
| ATOM | 15680 | HH12 | ARG | A1274 | 135.877 | 175.230 | 182.681 | 1.00 | 5.16 | H |
| ATOM | 15681 | HH21 | ARG | A1274 | 136.551 | 178.356 | 183.474 | 1.00 | 5.16 | H |
| ATOM | 15682 | HH22 | ARG | A1274 | 136.582 | 176.961 | 183.995 | 1.00 | 5.16 | H |
| ATOM | 15683 | N | ILE | A1275 | 138.870 | 177.002 | 176.508 | 1.00 | 4.40 | N |
| ATOM | 15684 | CA | ILE | A1275 | 139.939 | 177.906 | 176.092 | 1.00 | 4.40 | C |
| ATOM | 15685 | C | ILE | A1275 | 140.562 | 177.495 | 174.766 | 1.00 | 4.40 | C |
| ATOM | 15686 | O | ILE | A1275 | 141.210 | 178.327 | 174.112 | 1.00 | 4.40 | O |
| ATOM | 15687 | CB | ILE | A1275 | 141.038 | 177.982 | 177.172 | 1.00 | 4.40 | C |
| ATOM | 15688 | CG1 | ILE | A1275 | 140.453 | 178.443 | 178.512 | 1.00 | 4.40 | C |
| ATOM | 15689 | CG2 | ILE | A1275 | 142.155 | 178.921 | 176.740 | 1.00 | 4.40 | C |
| ATOM | 15690 | CD1 | ILE | A1275 | 139.865 | 179.845 | 178.494 | 1.00 | 4.40 | C |
| ATOM | 15691 | H | ILE | A1275 | 139.140 | 176.317 | 176.952 | 1.00 | 4.40 | H |
| ATOM | 15692 | HA | ILE | A1275 | 139.572 | 178.795 | 175.975 | 1.00 | 4.40 | H |
| ATOM | 15693 | HB | ILE | A1275 | 141.413 | 177.095 | 177.291 | 1.00 | 4.40 | H |
| ATOM | 15694 | HG12 | ILE | A1275 | 139.748 | 177.831 | 178.773 | 1.00 | 4.40 | H |
| ATOM | 15695 | HG13 | ILE | A1275 | 141.158 | 178.430 | 179.178 | 1.00 | 4.40 | H |
| ATOM | 15696 | HG21 | ILE | A1275 | 142.736 | 179.080 | 177.498 | 1.00 | 4.40 | H |
| ATOM | 15697 | HG22 | ILE | A1275 | 142.662 | 178.511 | 176.023 | 1.00 | 4.40 | H |
| ATOM | 15698 | HG23 | ILE | A1275 | 141.766 | 179.757 | 176.439 | 1.00 | 4.40 | H |
| ATOM | 15699 | HD11 | ILE | A1275 | 139.438 | 180.016 | 179.348 | 1.00 | 4.40 | H |
| ATOM | 15700 | HD12 | ILE | A1275 | 140.579 | 180.485 | 178.350 | 1.00 | 4.40 | H |
| ATOM | 15701 | HD13 | ILE | A1275 | 139.209 | 179.913 | 177.784 | 1.00 | 4.40 | H |
| ATOM | 15702 | N | LEU | A1276 | 140.377 | 176.245 | 174.338 | 1.00 | 4.77 | N |
| ATOM | 15703 | CA | LEU | A1276 | 140.971 | 175.772 | 173.095 | 1.00 | 4.77 | C |
| ATOM | 15704 | C | LEU | A1276 | 140.300 | 176.363 | 171.861 | 1.00 | 4.77 | C |
| ATOM | 15705 | O | LEU | A1276 | 140.857 | 176.264 | 170.763 | 1.00 | 4.77 | O |
| ATOM | 15706 | CB | LEU | A1276 | 140.902 | 174.244 | 173.035 | 1.00 | 4.77 | C |
| ATOM | 15707 | CG | LEU | A1276 | 141.833 | 173.491 | 173.992 | 1.00 | 4.77 | C |
| ATOM | 15708 | CD1 | LEU | A1276 | 141.512 | 172.004 | 173.995 | 1.00 | 4.77 | C |
| ATOM | 15709 | CD2 | LEU | A1276 | 143.293 | 173.718 | 173.627 | 1.00 | 4.77 | C |
| ATOM | 15710 | H | LEU | A1276 | 139.911 | 175.653 | 174.753 | 1.00 | 4.77 | H |
| ATOM | 15711 | HA | LEU | A1276 | 141.904 | 176.032 | 173.076 | 1.00 | 4.77 | H |
| ATOM | 15712 | HB2 | LEU | A1276 | 139.994 | 173.971 | 173.241 | 1.00 | 4.77 | H |
| ATOM | 15713 | HB3 | LEU | A1276 | 141.127 | 173.963 | 172.134 | 1.00 | 4.77 | H |
| ATOM | 15714 | HG | LEU | A1276 | 141.695 | 173.826 | 174.892 | 1.00 | 4.77 | H |
| ATOM | 15715 | HD11 | LEU | A1276 | 142.112 | 171.554 | 174.610 | 1.00 | 4.77 | H |
| ATOM | 15716 | HD12 | LEU | A1276 | 140.593 | 171.880 | 174.279 | 1.00 | 4.77 | H |
| ATOM | 15717 | HD13 | LEU | A1276 | 141.632 | 171.653 | 173.099 | 1.00 | 4.77 | H |
| ATOM | 15718 | HD21 | LEU | A1276 | 143.847 | 173.143 | 174.177 | 1.00 | 4.77 | H |
| ATOM | 15719 | HD22 | LEU | A1276 | 143.420 | 173.503 | 172.690 | 1.00 | 4.77 | H |
| ATOM | 15720 | HD23 | LEU | A1276 | 143.519 | 174.647 | 173.788 | 1.00 | 4.77 | H |
| ATOM | 15721 | N | ARG | A1277 | 139.122 | 176.970 | 172.016 | 1.00 | 7.12 | N |
| ATOM | 15722 | CA | ARG | A1277 | 138.421 | 177.576 | 170.890 | 1.00 | 7.12 | C |
| ATOM | 15723 | C | ARG | A1277 | 139.115 | 178.826 | 170.363 | 1.00 | 7.12 | C |
| ATOM | 15724 | O | ARG | A1277 | 138.751 | 179.304 | 169.283 | 1.00 | 7.12 | O |
| ATOM | 15725 | CB | ARG | A1277 | 136.988 | 177.925 | 171.299 | 1.00 | 7.12 | C |
| ATOM | 15726 | CG | ARG | A1277 | 136.221 | 176.771 | 171.925 | 1.00 | 7.12 | C |
| ATOM | 15727 | CD | ARG | A1277 | 134.820 | 177.192 | 172.336 | 1.00 | 7.12 | C |
| ATOM | 15728 | NE | ARG | A1277 | 134.054 | 177.728 | 171.213 | 1.00 | 7.12 | N |
| ATOM | 15729 | CZ | ARG | A1277 | 133.116 | 178.666 | 171.314 | 1.00 | 7.12 | C |
| ATOM | 15730 | NH1 | ARG | A1277 | 132.480 | 179.081 | 170.227 | 1.00 | 7.12 | N |
| ATOM | 15731 | NH2 | ARG | A1277 | 132.804 | 179.189 | 172.494 | 1.00 | 7.12 | N |
| ATOM | 15732 | H | ARG | A1277 | 138.710 | 177.044 | 172.766 | 1.00 | 7.12 | H |
| ATOM | 15733 | HA | ARG | A1277 | 138.376 | 176.933 | 170.166 | 1.00 | 7.12 | H |
| ATOM | 15734 | HB2 | ARG | A1277 | 137.017 | 178.647 | 171.947 | 1.00 | 7.12 | H |
| ATOM | 15735 | HB3 | ARG | A1277 | 136.502 | 178.212 | 170.510 | 1.00 | 7.12 | H |
| ATOM | 15736 | HG2 | ARG | A1277 | 136.144 | 176.051 | 171.280 | 1.00 | 7.12 | H |
| ATOM | 15737 | HG3 | ARG | A1277 | 136.688 | 176.464 | 172.718 | 1.00 | 7.12 | H |
| ATOM | 15738 | HD2 | ARG | A1277 | 134.346 | 176.423 | 172.688 | 1.00 | 7.12 | H |
| ATOM | 15739 | HD3 | ARG | A1277 | 134.894 | 177.879 | 173.016 | 1.00 | 7.12 | H |
| ATOM | 15740 | HE | ARG | A1277 | 134.213 | 177.405 | 170.432 | 1.00 | 7.12 | H |
| ATOM | 15741 | HH11 | ARG | A1277 | 132.677 | 178.746 | 169.460 | 1.00 | 7.12 | H |
| ATOM | 15742 | HH12 | ARG | A1277 | 131.872 | 179.686 | 170.289 | 1.00 | 7.12 | H |
| ATOM | 15743 | HH21 | ARG | A1277 | 133.207 | 178.929 | 173.207 | 1.00 | 7.12 | H |
| ATOM | 15744 | HH22 | ARG | A1277 | 132.194 | 179.794 | 172.545 | 1.00 | 7.12 | H |
| ATOM | 15745 | N | LEU | A1278 | 140.095 | 179.363 | 171.092 | 1.00 | 6.90 | N |
| ATOM | 15746 | CA | LEU | A1278 | 140.737 | 180.607 | 170.681 | 1.00 | 6.90 | C |
| ATOM | 15747 | C | LEU | A1278 | 141.469 | 180.477 | 169.352 | 1.00 | 6.90 | C |
| ATOM | 15748 | O | LEU | A1278 | 141.663 | 181.485 | 168.664 | 1.00 | 6.90 | O |
| ATOM | 15749 | CB | LEU | A1278 | 141.711 | 181.069 | 171.766 | 1.00 | 6.90 | C |
| ATOM | 15750 | CG | LEU | A1278 | 142.255 | 182.491 | 171.625 | 1.00 | 6.90 | C |
| ATOM | 15751 | CD1 | LEU | A1278 | 141.148 | 183.523 | 171.779 | 1.00 | 6.90 | C |
| ATOM | 15752 | CD2 | LEU | A1278 | 143.359 | 182.733 | 172.640 | 1.00 | 6.90 | C |
| ATOM | 15753 | H | LEU | A1278 | 140.404 | 179.029 | 171.821 | 1.00 | 6.90 | H |
| ATOM | 15754 | HA | LEU | A1278 | 140.055 | 181.288 | 170.579 | 1.00 | 6.90 | H |
| ATOM | 15755 | HB2 | LEU | A1278 | 141.259 | 181.016 | 172.623 | 1.00 | 6.90 | H |
| ATOM | 15756 | HB3 | LEU | A1278 | 142.472 | 180.467 | 171.767 | 1.00 | 6.90 | H |
| ATOM | 15757 | HG | LEU | A1278 | 142.638 | 182.595 | 170.740 | 1.00 | 6.90 | H |
| ATOM | 15758 | HD11 | LEU | A1278 | 141.540 | 184.410 | 171.750 | 1.00 | 6.90 | H |
| ATOM | 15759 | HD12 | LEU | A1278 | 140.514 | 183.421 | 171.052 | 1.00 | 6.90 | H |
| ATOM | 15760 | HD13 | LEU | A1278 | 140.703 | 183.385 | 172.630 | 1.00 | 6.90 | H |
| ATOM | 15761 | HD21 | LEU | A1278 | 143.745 | 183.608 | 172.483 | 1.00 | 6.90 | H |
| ATOM | 15762 | HD22 | LEU | A1278 | 142.981 | 182.692 | 173.532 | 1.00 | 6.90 | H |
| ATOM | 15763 | HD23 | LEU | A1278 | 144.038 | 182.048 | 172.536 | 1.00 | 6.90 | H |
| ATOM | 15764 | N | VAL | A1279 | 141.878 | 179.265 | 168.971 | 1.00 | 7.19 | N |
| ATOM | 15765 | CA | VAL | A1279 | 142.590 | 179.087 | 167.711 | 1.00 | 7.19 | C |
| ATOM | 15766 | C | VAL | A1279 | 141.667 | 179.208 | 166.505 | 1.00 | 7.19 | C |
| ATOM | 15767 | O | VAL | A1279 | 142.153 | 179.302 | 165.371 | 1.00 | 7.19 | O |
| ATOM | 15768 | CB | VAL | A1279 | 143.309 | 177.726 | 167.682 | 1.00 | 7.19 | C |
| ATOM | 15769 | CG1 | VAL | A1279 | 144.203 | 177.572 | 168.905 | 1.00 | 7.19 | C |
| ATOM | 15770 | CG2 | VAL | A1279 | 142.300 | 176.589 | 167.605 | 1.00 | 7.19 | C |
| ATOM | 15771 | H | VAL | A1279 | 141.757 | 178.542 | 169.420 | 1.00 | 7.19 | H |
| ATOM | 15772 | HA | VAL | A1279 | 143.266 | 179.779 | 167.634 | 1.00 | 7.19 | H |
| ATOM | 15773 | HB | VAL | A1279 | 143.870 | 177.682 | 166.892 | 1.00 | 7.19 | H |
| ATOM | 15774 | HG11 | VAL | A1279 | 144.725 | 176.761 | 168.813 | 1.00 | 7.19 | H |
| ATOM | 15775 | HG12 | VAL | A1279 | 144.792 | 178.341 | 168.962 | 1.00 | 7.19 | H |
| ATOM | 15776 | HG13 | VAL | A1279 | 143.649 | 177.521 | 169.699 | 1.00 | 7.19 | H |
| ATOM | 15777 | HG21 | VAL | A1279 | 142.745 | 175.759 | 167.839 | 1.00 | 7.19 | H |
| ATOM | 15778 | HG22 | VAL | A1279 | 141.578 | 176.763 | 168.229 | 1.00 | 7.19 | H |
| ATOM | 15779 | HG23 | VAL | A1279 | 141.951 | 176.534 | 166.702 | 1.00 | 7.19 | H |
| ATOM | 15780 | N | LYS | A1280 | 140.353 | 179.204 | 166.715 | 1.00 | 11.33 | N |
| ATOM | 15781 | CA | LYS | A1280 | 139.415 | 179.277 | 165.602 | 1.00 | 11.33 | C |
| ATOM | 15782 | C | LYS | A1280 | 139.597 | 180.576 | 164.825 | 1.00 | 11.33 | C |
| ATOM | 15783 | O | LYS | A1280 | 139.646 | 181.663 | 165.408 | 1.00 | 11.33 | O |
| ATOM | 15784 | CB | LYS | A1280 | 137.982 | 179.169 | 166.122 | 1.00 | 11.33 | C |
| ATOM | 15785 | CG | LYS | A1280 | 136.987 | 178.658 | 165.093 | 1.00 | 11.33 | C |
| ATOM | 15786 | CD | LYS | A1280 | 135.677 | 179.434 | 165.129 | 1.00 | 11.33 | C |
| ATOM | 15787 | CE | LYS | A1280 | 134.487 | 178.523 | 164.881 | 1.00 | 11.33 | C |
| ATOM | 15788 | NZ | LYS | A1280 | 133.186 | 179.220 | 165.078 | 1.00 | 11.33 | N |
| ATOM | 15789 | H | LYS | A1280 | 139.978 | 179.160 | 167.488 | 1.00 | 11.33 | H |
| ATOM | 15790 | HA | LYS | A1280 | 139.577 | 178.536 | 164.997 | 1.00 | 11.33 | H |
| ATOM | 15791 | HB2 | LYS | A1280 | 137.969 | 178.557 | 166.874 | 1.00 | 11.33 | H |
| ATOM | 15792 | HB3 | LYS | A1280 | 137.693 | 180.049 | 166.409 | 1.00 | 11.33 | H |
| ATOM | 15793 | HG2 | LYS | A1280 | 137.370 | 178.752 | 164.207 | 1.00 | 11.33 | H |
| ATOM | 15794 | HG3 | LYS | A1280 | 136.792 | 177.727 | 165.275 | 1.00 | 11.33 | H |
| ATOM | 15795 | HD2 | LYS | A1280 | 135.564 | 179.843 | 166.002 | 1.00 | 11.33 | H |
| ATOM | 15796 | HD3 | LYS | A1280 | 135.688 | 180.115 | 164.439 | 1.00 | 11.33 | H |
| ATOM | 15797 | HE2 | LYS | A1280 | 134.520 | 178.191 | 163.970 | 1.00 | 11.33 | H |
| ATOM | 15798 | HE3 | LYS | A1280 | 134.525 | 177.784 | 165.507 | 1.00 | 11.33 | H |
| ATOM | 15799 | HZ1 | LYS | A1280 | 132.513 | 178.657 | 164.927 | 1.00 | 11.33 | H |
| ATOM | 15800 | HZ2 | LYS | A1280 | 133.128 | 179.523 | 165.912 | 1.00 | 11.33 | H |
| ATOM | 15801 | HZ3 | LYS | A1280 | 133.119 | 179.907 | 164.515 | 1.00 | 11.33 | H |
| ATOM | 15802 | N | GLY | A1281 | 139.695 | 180.457 | 163.501 | 1.00 | 12.86 | N |
| ATOM | 15803 | CA | GLY | A1281 | 139.755 | 181.592 | 162.609 | 1.00 | 12.86 | C |
| ATOM | 15804 | C | GLY | A1281 | 141.153 | 182.010 | 162.201 | 1.00 | 12.86 | C |
| ATOM | 15805 | O | GLY | A1281 | 141.307 | 182.680 | 161.173 | 1.00 | 12.86 | O |
| ATOM | 15806 | H | GLY | A1281 | 139.729 | 179.701 | 163.093 | 1.00 | 12.86 | H |
| ATOM | 15807 | HA2 | GLY | A1281 | 139.258 | 181.384 | 161.803 | 1.00 | 12.86 | H |
| ATOM | 15808 | HA3 | GLY | A1281 | 139.328 | 182.351 | 163.036 | 1.00 | 12.86 | H |
| ATOM | 15809 | N | ALA | A1282 | 142.172 | 181.635 | 162.969 | 1.00 | 11.28 | N |
| ATOM | 15810 | CA | ALA | A1282 | 143.540 | 182.006 | 162.632 | 1.00 | 11.28 | C |
| ATOM | 15811 | C | ALA | A1282 | 143.982 | 181.290 | 161.361 | 1.00 | 11.28 | C |
| ATOM | 15812 | O | ALA | A1282 | 143.726 | 180.094 | 161.190 | 1.00 | 11.28 | O |
| ATOM | 15813 | CB | ALA | A1282 | 144.477 | 181.669 | 163.789 | 1.00 | 11.28 | C |
| ATOM | 15814 | H | ALA | A1282 | 142.098 | 181.168 | 163.688 | 1.00 | 11.28 | H |
| ATOM | 15815 | HA | ALA | A1282 | 143.583 | 182.962 | 162.474 | 1.00 | 11.28 | H |
| ATOM | 15816 | HB1 | ALA | A1282 | 145.391 | 181.853 | 163.520 | 1.00 | 11.28 | H |
| ATOM | 15817 | HB2 | ALA | A1282 | 144.240 | 182.213 | 164.556 | 1.00 | 11.28 | H |
| ATOM | 15818 | HB3 | ALA | A1282 | 144.378 | 180.729 | 164.006 | 1.00 | 11.28 | H |
| ATOM | 15819 | N | LYS | A1283 | 144.650 | 182.023 | 160.468 | 1.00 | 13.19 | N |
| ATOM | 15820 | CA | LYS | A1283 | 145.006 | 181.467 | 159.166 | 1.00 | 13.19 | C |
| ATOM | 15821 | C | LYS | A1283 | 146.316 | 180.686 | 159.221 | 1.00 | 13.19 | C |
| ATOM | 15822 | O | LYS | A1283 | 146.365 | 179.522 | 158.814 | 1.00 | 13.19 | O |
| ATOM | 15823 | CB | LYS | A1283 | 145.093 | 182.587 | 158.127 | 1.00 | 13.19 | C |
| ATOM | 15824 | CG | LYS | A1283 | 143.812 | 183.403 | 157.973 | 1.00 | 13.19 | C |
| ATOM | 15825 | CD | LYS | A1283 | 142.621 | 182.550 | 157.549 | 1.00 | 13.19 | C |
| ATOM | 15826 | CE | LYS | A1283 | 142.772 | 182.032 | 156.128 | 1.00 | 13.19 | C |
| ATOM | 15827 | NZ | LYS | A1283 | 141.541 | 181.336 | 155.660 | 1.00 | 13.19 | N |
| ATOM | 15828 | H | LYS | A1283 | 144.906 | 182.835 | 160.592 | 1.00 | 13.19 | H |
| ATOM | 15829 | HA | LYS | A1283 | 144.314 | 180.849 | 158.888 | 1.00 | 13.19 | H |
| ATOM | 15830 | HB2 | LYS | A1283 | 145.800 | 183.197 | 158.388 | 1.00 | 13.19 | H |
| ATOM | 15831 | HB3 | LYS | A1283 | 145.301 | 182.196 | 157.264 | 1.00 | 13.19 | H |
| ATOM | 15832 | HG2 | LYS | A1283 | 143.596 | 183.818 | 158.823 | 1.00 | 13.19 | H |
| ATOM | 15833 | HG3 | LYS | A1283 | 143.950 | 184.084 | 157.296 | 1.00 | 13.19 | H |
| ATOM | 15834 | HD2 | LYS | A1283 | 142.530 | 181.790 | 158.143 | 1.00 | 13.19 | H |
| ATOM | 15835 | HD3 | LYS | A1283 | 141.818 | 183.092 | 157.584 | 1.00 | 13.19 | H |
| ATOM | 15836 | HE2 | LYS | A1283 | 142.943 | 182.778 | 155.532 | 1.00 | 13.19 | H |
| ATOM | 15837 | HE3 | LYS | A1283 | 143.508 | 181.400 | 156.094 | 1.00 | 13.19 | H |
| ATOM | 15838 | HZ1 | LYS | A1283 | 141.654 | 181.045 | 154.826 | 1.00 | 13.19 | H |
| ATOM | 15839 | HZ2 | LYS | A1283 | 141.367 | 180.640 | 156.187 | 1.00 | 13.19 | H |
| ATOM | 15840 | HZ3 | LYS | A1283 | 140.849 | 181.895 | 155.681 | 1.00 | 13.19 | H |
| ATOM | 15841 | N | GLY | A1284 | 147.390 | 181.311 | 159.706 | 1.00 | 8.89 | N |
| ATOM | 15842 | CA | GLY | A1284 | 148.658 | 180.601 | 159.805 | 1.00 | 8.89 | C |
| ATOM | 15843 | C | GLY | A1284 | 148.614 | 179.472 | 160.817 | 1.00 | 8.89 | C |
| ATOM | 15844 | O | GLY | A1284 | 149.165 | 178.388 | 160.585 | 1.00 | 8.89 | O |
| ATOM | 15845 | H | GLY | A1284 | 147.410 | 182.126 | 159.978 | 1.00 | 8.89 | H |
| ATOM | 15846 | HA2 | GLY | A1284 | 148.888 | 180.229 | 158.940 | 1.00 | 8.89 | H |
| ATOM | 15847 | HA3 | GLY | A1284 | 149.355 | 181.222 | 160.068 | 1.00 | 8.89 | H |
| ATOM | 15848 | N | ILE | A1285 | 147.968 | 179.711 | 161.960 | 1.00 | 7.17 | N |
| ATOM | 15849 | CA | ILE | A1285 | 147.792 | 178.650 | 162.944 | 1.00 | 7.17 | C |
| ATOM | 15850 | C | ILE | A1285 | 146.986 | 177.510 | 162.340 | 1.00 | 7.17 | C |
| ATOM | 15851 | O | ILE | A1285 | 147.287 | 176.332 | 162.563 | 1.00 | 7.17 | O |
| ATOM | 15852 | CB | ILE | A1285 | 147.130 | 179.209 | 164.218 | 1.00 | 7.17 | C |
| ATOM | 15853 | CG1 | ILE | A1285 | 148.074 | 180.218 | 164.889 | 1.00 | 7.17 | C |
| ATOM | 15854 | CG2 | ILE | A1285 | 146.737 | 178.066 | 165.165 | 1.00 | 7.17 | C |
| ATOM | 15855 | CD1 | ILE | A1285 | 147.884 | 180.384 | 166.387 | 1.00 | 7.17 | C |
| ATOM | 15856 | H | ILE | A1285 | 147.628 | 180.469 | 162.184 | 1.00 | 7.17 | H |
| ATOM | 15857 | HA | ILE | A1285 | 148.663 | 178.300 | 163.188 | 1.00 | 7.17 | H |
| ATOM | 15858 | HB | ILE | A1285 | 146.323 | 179.676 | 163.957 | 1.00 | 7.17 | H |
| ATOM | 15859 | HG12 | ILE | A1285 | 148.988 | 179.930 | 164.741 | 1.00 | 7.17 | H |
| ATOM | 15860 | HG13 | ILE | A1285 | 147.937 | 181.086 | 164.479 | 1.00 | 7.17 | H |
| ATOM | 15861 | HG21 | ILE | A1285 | 146.320 | 178.429 | 165.961 | 1.00 | 7.17 | H |
| ATOM | 15862 | HG22 | ILE | A1285 | 146.105 | 177.480 | 164.722 | 1.00 | 7.17 | H |
| ATOM | 15863 | HG23 | ILE | A1285 | 147.534 | 177.569 | 165.403 | 1.00 | 7.17 | H |
| ATOM | 15864 | HD11 | ILE | A1285 | 148.395 | 181.152 | 166.684 | 1.00 | 7.17 | H |
| ATOM | 15865 | HD12 | ILE | A1285 | 146.942 | 180.523 | 166.572 | 1.00 | 7.17 | H |
| ATOM | 15866 | HD13 | ILE | A1285 | 148.197 | 179.584 | 166.838 | 1.00 | 7.17 | H |
| ATOM | 15867 | N | ARG | A1286 | 145.948 | 177.840 | 161.567 | 1.00 | 9.89 | N |
| ATOM | 15868 | CA | ARG | A1286 | 145.172 | 176.804 | 160.893 | 1.00 | 9.89 | C |
| ATOM | 15869 | C | ARG | A1286 | 146.037 | 176.030 | 159.908 | 1.00 | 9.89 | C |
| ATOM | 15870 | O | ARG | A1286 | 145.898 | 174.811 | 159.776 | 1.00 | 9.89 | O |
| ATOM | 15871 | CB | ARG | A1286 | 143.970 | 177.424 | 160.179 | 1.00 | 9.89 | C |
| ATOM | 15872 | CG | ARG | A1286 | 143.231 | 176.470 | 159.242 | 1.00 | 9.89 | C |
| ATOM | 15873 | CD | ARG | A1286 | 142.142 | 177.183 | 158.460 | 1.00 | 9.89 | C |
| ATOM | 15874 | NE | ARG | A1286 | 140.903 | 176.411 | 158.421 | 1.00 | 9.89 | N |
| ATOM | 15875 | CZ | ARG | A1286 | 140.623 | 175.478 | 157.514 | 1.00 | 9.89 | C |
| ATOM | 15876 | NH1 | ARG | A1286 | 139.466 | 174.833 | 157.566 | 1.00 | 9.89 | N |
| ATOM | 15877 | NH2 | ARG | A1286 | 141.495 | 175.183 | 156.558 | 1.00 | 9.89 | N |
| ATOM | 15878 | H | ARG | A1286 | 145.678 | 178.644 | 161.420 | 1.00 | 9.89 | H |
| ATOM | 15879 | HA | ARG | A1286 | 144.839 | 176.178 | 161.555 | 1.00 | 9.89 | H |
| ATOM | 15880 | HB2 | ARG | A1286 | 143.335 | 177.726 | 160.847 | 1.00 | 9.89 | H |
| ATOM | 15881 | HB3 | ARG | A1286 | 144.275 | 178.178 | 159.651 | 1.00 | 9.89 | H |
| ATOM | 15882 | HG2 | ARG | A1286 | 143.849 | 176.092 | 158.598 | 1.00 | 9.89 | H |
| ATOM | 15883 | HG3 | ARG | A1286 | 142.818 | 175.765 | 159.764 | 1.00 | 9.89 | H |
| ATOM | 15884 | HD2 | ARG | A1286 | 141.952 | 178.032 | 158.888 | 1.00 | 9.89 | H |
| ATOM | 15885 | HD3 | ARG | A1286 | 142.444 | 177.333 | 157.550 | 1.00 | 9.89 | H |
| ATOM | 15886 | HE | ARG | A1286 | 140.312 | 176.572 | 159.025 | 1.00 | 9.89 | H |
| ATOM | 15887 | HH11 | ARG | A1286 | 138.897 | 175.018 | 158.183 | 1.00 | 9.89 | H |
| ATOM | 15888 | HH12 | ARG | A1286 | 139.285 | 174.229 | 156.981 | 1.00 | 9.89 | H |
| ATOM | 15889 | HH21 | ARG | A1286 | 142.249 | 175.594 | 156.514 | 1.00 | 9.89 | H |
| ATOM | 15890 | HH22 | ARG | A1286 | 141.304 | 174.578 | 155.978 | 1.00 | 9.89 | H |
| ATOM | 15891 | N | LEU | A1287 | 146.922 | 176.724 | 159.193 | 1.00 | 7.01 | N |
| ATOM | 15892 | CA | LEU | A1287 | 147.808 | 176.051 | 158.248 | 1.00 | 7.01 | C |
| ATOM | 15893 | C | LEU | A1287 | 148.700 | 175.043 | 158.963 | 1.00 | 7.01 | C |
| ATOM | 15894 | O | LEU | A1287 | 148.821 | 173.883 | 158.543 | 1.00 | 7.01 | O |
| ATOM | 15895 | CB | LEU | A1287 | 148.652 | 177.094 | 157.512 | 1.00 | 7.01 | C |
| ATOM | 15896 | CG | LEU | A1287 | 149.063 | 176.789 | 156.072 | 1.00 | 7.01 | C |
| ATOM | 15897 | CD1 | LEU | A1287 | 147.851 | 176.712 | 155.156 | 1.00 | 7.01 | C |
| ATOM | 15898 | CD2 | LEU | A1287 | 150.051 | 177.832 | 155.570 | 1.00 | 7.01 | C |
| ATOM | 15899 | H | LEU | A1287 | 147.029 | 177.576 | 159.238 | 1.00 | 7.01 | H |
| ATOM | 15900 | HA | LEU | A1287 | 147.273 | 175.572 | 157.596 | 1.00 | 7.01 | H |
| ATOM | 15901 | HB2 | LEU | A1287 | 148.152 | 177.925 | 157.492 | 1.00 | 7.01 | H |
| ATOM | 15902 | HB3 | LEU | A1287 | 149.469 | 177.227 | 158.017 | 1.00 | 7.01 | H |
| ATOM | 15903 | HG | LEU | A1287 | 149.507 | 175.930 | 156.057 | 1.00 | 7.01 | H |
| ATOM | 15904 | HD11 | LEU | A1287 | 148.155 | 176.660 | 154.237 | 1.00 | 7.01 | H |
| ATOM | 15905 | HD12 | LEU | A1287 | 147.334 | 175.922 | 155.378 | 1.00 | 7.01 | H |
| ATOM | 15906 | HD13 | LEU | A1287 | 147.311 | 177.509 | 155.281 | 1.00 | 7.01 | H |
| ATOM | 15907 | HD21 | LEU | A1287 | 150.301 | 177.617 | 154.658 | 1.00 | 7.01 | H |
| ATOM | 15908 | HD22 | LEU | A1287 | 149.631 | 178.706 | 155.601 | 1.00 | 7.01 | H |
| ATOM | 15909 | HD23 | LEU | A1287 | 150.837 | 177.822 | 156.139 | 1.00 | 7.01 | H |
| ATOM | 15910 | N | LEU | A1288 | 149.330 | 175.472 | 160.058 | 1.00 | 4.51 | N |
| ATOM | 15911 | CA | LEU | A1288 | 150.204 | 174.568 | 160.802 | 1.00 | 4.51 | C |
| ATOM | 15912 | C | LEU | A1288 | 149.416 | 173.410 | 161.407 | 1.00 | 4.51 | C |
| ATOM | 15913 | O | LEU | A1288 | 149.890 | 172.267 | 161.420 | 1.00 | 4.51 | O |
| ATOM | 15914 | CB | LEU | A1288 | 150.954 | 175.339 | 161.887 | 1.00 | 4.51 | C |
| ATOM | 15915 | CG | LEU | A1288 | 151.990 | 176.336 | 161.363 | 1.00 | 4.51 | C |
| ATOM | 15916 | CD1 | LEU | A1288 | 152.324 | 177.379 | 162.418 | 1.00 | 4.51 | C |
| ATOM | 15917 | CD2 | LEU | A1288 | 153.248 | 175.612 | 160.903 | 1.00 | 4.51 | C |
| ATOM | 15918 | H | LEU | A1288 | 149.269 | 176.265 | 160.384 | 1.00 | 4.51 | H |
| ATOM | 15919 | HA | LEU | A1288 | 150.860 | 174.195 | 160.193 | 1.00 | 4.51 | H |
| ATOM | 15920 | HB2 | LEU | A1288 | 150.310 | 175.835 | 162.416 | 1.00 | 4.51 | H |
| ATOM | 15921 | HB3 | LEU | A1288 | 151.420 | 174.702 | 162.453 | 1.00 | 4.51 | H |
| ATOM | 15922 | HG | LEU | A1288 | 151.618 | 176.799 | 160.596 | 1.00 | 4.51 | H |
| ATOM | 15923 | HD11 | LEU | A1288 | 152.952 | 178.016 | 162.044 | 1.00 | 4.51 | H |
| ATOM | 15924 | HD12 | LEU | A1288 | 151.509 | 177.833 | 162.682 | 1.00 | 4.51 | H |
| ATOM | 15925 | HD13 | LEU | A1288 | 152.719 | 176.935 | 163.185 | 1.00 | 4.51 | H |
| ATOM | 15926 | HD21 | LEU | A1288 | 153.905 | 176.268 | 160.621 | 1.00 | 4.51 | H |
| ATOM | 15927 | HD22 | LEU | A1288 | 153.598 | 175.088 | 161.640 | 1.00 | 4.51 | H |
| ATOM | 15928 | HD23 | LEU | A1288 | 153.023 | 175.029 | 160.160 | 1.00 | 4.51 | H |
| ATOM | 15929 | N | LEU | A1289 | 148.212 | 173.684 | 161.914 | 1.00 | 3.68 | N |
| ATOM | 15930 | CA | LEU | A1289 | 147.391 | 172.624 | 162.488 | 1.00 | 3.68 | C |
| ATOM | 15931 | C | LEU | A1289 | 146.961 | 171.623 | 161.424 | 1.00 | 3.68 | C |
| ATOM | 15932 | O | LEU | A1289 | 146.898 | 170.420 | 161.689 | 1.00 | 3.68 | O |
| ATOM | 15933 | CB | LEU | A1289 | 146.171 | 173.229 | 163.183 | 1.00 | 3.68 | C |
| ATOM | 15934 | CG | LEU | A1289 | 146.438 | 174.144 | 164.382 | 1.00 | 3.68 | C |
| ATOM | 15935 | CD1 | LEU | A1289 | 145.138 | 174.479 | 165.097 | 1.00 | 3.68 | C |
| ATOM | 15936 | CD2 | LEU | A1289 | 147.436 | 173.518 | 165.348 | 1.00 | 3.68 | C |
| ATOM | 15937 | H | LEU | A1289 | 147.854 | 174.466 | 161.936 | 1.00 | 3.68 | H |
| ATOM | 15938 | HA | LEU | A1289 | 147.913 | 172.144 | 163.148 | 1.00 | 3.68 | H |
| ATOM | 15939 | N | LEU | A1290 | 146.649 | 172.099 | 160.217 | 1.00 | 5.20 | N |
| ATOM | 15940 | CA | LEU | A1290 | 146.327 | 171.194 | 159.119 | 1.00 | 5.20 | C |
| ATOM | 15941 | C | LEU | A1290 | 147.524 | 170.322 | 158.765 | 1.00 | 5.20 | C |
| ATOM | 15942 | O | LEU | A1290 | 147.381 | 169.112 | 158.541 | 1.00 | 5.20 | O |
| ATOM | 15943 | CB | LEU | A1290 | 145.869 | 172.001 | 157.903 | 1.00 | 5.20 | C |
| ATOM | 15944 | CG | LEU | A1290 | 145.769 | 171.264 | 156.565 | 1.00 | 5.20 | C |
| ATOM | 15945 | CD1 | LEU | A1290 | 144.792 | 170.105 | 156.660 | 1.00 | 5.20 | C |
| ATOM | 15946 | CD2 | LEU | A1290 | 145.362 | 172.224 | 155.458 | 1.00 | 5.20 | C |
| ATOM | 15947 | H | LEU | A1290 | 146.616 | 172.934 | 160.012 | 1.00 | 5.20 | H |
| ATOM | 15948 | HA | LEU | A1290 | 145.598 | 170.613 | 159.388 | 1.00 | 5.20 | H |
| ATOM | 15949 | HB2 | LEU | A1290 | 144.989 | 172.361 | 158.095 | 1.00 | 5.20 | H |
| ATOM | 15950 | HB3 | LEU | A1290 | 146.492 | 172.734 | 157.777 | 1.00 | 5.20 | H |
| ATOM | 15951 | HG | LEU | A1290 | 146.639 | 170.902 | 156.334 | 1.00 | 5.20 | H |
| ATOM | 15952 | HD11 | LEU | A1290 | 144.698 | 169.700 | 155.784 | 1.00 | 5.20 | H |
| ATOM | 15953 | HD12 | LEU | A1290 | 145.135 | 169.453 | 157.292 | 1.00 | 5.20 | H |
| ATOM | 15954 | HD13 | LEU | A1290 | 143.934 | 170.441 | 156.963 | 1.00 | 5.20 | H |
| ATOM | 15955 | HD21 | LEU | A1290 | 145.249 | 171.725 | 154.634 | 1.00 | 5.20 | H |
| ATOM | 15956 | HD22 | LEU | A1290 | 144.528 | 172.653 | 155.704 | 1.00 | 5.20 | H |
| ATOM | 15957 | HD23 | LEU | A1290 | 146.058 | 172.891 | 155.349 | 1.00 | 5.20 | H |
| ATOM | 15958 | N | ALA | A1291 | 148.716 | 170.921 | 158.714 | 1.00 | 4.03 | N |
| ATOM | 15959 | CA | ALA | A1291 | 149.915 | 170.133 | 158.449 | 1.00 | 4.03 | C |
| ATOM | 15960 | C | ALA | A1291 | 150.102 | 169.056 | 159.511 | 1.00 | 4.03 | C |
| ATOM | 15961 | O | ALA | A1291 | 150.424 | 167.906 | 159.192 | 1.00 | 4.03 | O |
| ATOM | 15962 | CB | ALA | A1291 | 151.141 | 171.043 | 158.384 | 1.00 | 4.03 | C |
| ATOM | 15963 | H | ALA | A1291 | 148.854 | 171.763 | 158.826 | 1.00 | 4.03 | H |
| ATOM | 15964 | HA | ALA | A1291 | 149.821 | 169.693 | 157.589 | 1.00 | 4.03 | H |
| ATOM | 15965 | HB1 | ALA | A1291 | 151.926 | 170.501 | 158.209 | 1.00 | 4.03 | H |
| ATOM | 15966 | HB2 | ALA | A1291 | 151.018 | 171.688 | 157.671 | 1.00 | 4.03 | H |
| ATOM | 15967 | HB3 | ALA | A1291 | 151.238 | 171.502 | 159.233 | 1.00 | 4.03 | H |
| ATOM | 15968 | N | LEU | A1292 | 149.900 | 169.412 | 160.781 | 1.00 | 2.64 | N |
| ATOM | 15969 | CA | LEU | A1292 | 150.006 | 168.422 | 161.851 | 1.00 | 2.64 | C |
| ATOM | 15970 | C | LEU | A1292 | 148.950 | 167.332 | 161.701 | 1.00 | 2.64 | C |
| ATOM | 15971 | O | LEU | A1292 | 149.233 | 166.147 | 161.910 | 1.00 | 2.64 | O |
| ATOM | 15972 | CB | LEU | A1292 | 149.878 | 169.109 | 163.211 | 1.00 | 2.64 | C |
| ATOM | 15973 | CG | LEU | A1292 | 150.115 | 168.230 | 164.442 | 1.00 | 2.64 | C |
| ATOM | 15974 | CD1 | LEU | A1292 | 151.549 | 167.723 | 164.480 | 1.00 | 2.64 | C |
| ATOM | 15975 | CD2 | LEU | A1292 | 149.781 | 168.995 | 165.714 | 1.00 | 2.64 | C |
| ATOM | 15976 | H | LEU | A1292 | 149.705 | 170.206 | 161.046 | 1.00 | 2.64 | H |
| ATOM | 15977 | HA | LEU | A1292 | 150.879 | 168.003 | 161.805 | 1.00 | 2.64 | H |
| ATOM | 15978 | HB2 | LEU | A1292 | 150.522 | 169.833 | 163.247 | 1.00 | 2.64 | H |
| ATOM | 15979 | HB3 | LEU | A1292 | 148.982 | 169.472 | 163.285 | 1.00 | 2.64 | H |
| ATOM | 15980 | HG | LEU | A1292 | 149.528 | 167.459 | 164.396 | 1.00 | 2.64 | H |
| ATOM | 15981 | HD11 | LEU | A1292 | 151.691 | 167.245 | 165.312 | 1.00 | 2.64 | H |
| ATOM | 15982 | HD12 | LEU | A1292 | 151.695 | 167.127 | 163.728 | 1.00 | 2.64 | H |
| ATOM | 15983 | HD13 | LEU | A1292 | 152.153 | 168.480 | 164.426 | 1.00 | 2.64 | H |
| ATOM | 15984 | HD21 | LEU | A1292 | 149.923 | 168.414 | 166.478 | 1.00 | 2.64 | H |
| ATOM | 15985 | HD22 | LEU | A1292 | 150.359 | 169.771 | 165.776 | 1.00 | 2.64 | H |
| ATOM | 15986 | HD23 | LEU | A1292 | 148.853 | 169.275 | 165.678 | 1.00 | 2.64 | H |
| ATOM | 15987 | N | ARG | A1293 | 147.724 | 167.716 | 161.339 | 1.00 | 4.79 | N |
| ATOM | 15988 | CA | ARG | A1293 | 146.644 | 166.745 | 161.201 | 1.00 | 4.79 | C |
| ATOM | 15989 | C | ARG | A1293 | 146.949 | 165.735 | 160.105 | 1.00 | 4.79 | C |
| ATOM | 15990 | O | ARG | A1293 | 146.681 | 164.538 | 160.261 | 1.00 | 4.79 | O |
| ATOM | 15991 | CB | ARG | A1293 | 145.329 | 167.469 | 160.910 | 1.00 | 4.79 | C |
| ATOM | 15992 | CG | ARG | A1293 | 144.101 | 166.563 | 160.885 | 1.00 | 4.79 | C |
| ATOM | 15993 | CD | ARG | A1293 | 142.915 | 167.213 | 160.176 | 1.00 | 4.79 | C |
| ATOM | 15994 | NE | ARG | A1293 | 143.042 | 168.665 | 160.069 | 1.00 | 4.79 | N |
| ATOM | 15995 | CZ | ARG | A1293 | 142.344 | 169.417 | 159.222 | 1.00 | 4.79 | C |
| ATOM | 15996 | NH1 | ARG | A1293 | 141.462 | 168.863 | 158.401 | 1.00 | 4.79 | N |
| ATOM | 15997 | NH2 | ARG | A1293 | 142.528 | 170.730 | 159.198 | 1.00 | 4.79 | N |
| ATOM | 15998 | H | ARG | A1293 | 147.495 | 168.528 | 161.169 | 1.00 | 4.79 | H |
| ATOM | 15999 | HA | ARG | A1293 | 146.544 | 166.262 | 162.036 | 1.00 | 4.79 | H |
| ATOM | 16000 | HB2 | ARG | A1293 | 145.186 | 168.144 | 161.593 | 1.00 | 4.79 | H |
| ATOM | 16001 | HB3 | ARG | A1293 | 145.404 | 167.893 | 160.040 | 1.00 | 4.79 | H |
| ATOM | 16002 | HG2 | ARG | A1293 | 144.315 | 165.742 | 160.416 | 1.00 | 4.79 | H |
| ATOM | 16003 | HG3 | ARG | A1293 | 143.835 | 166.366 | 161.796 | 1.00 | 4.79 | H |
| ATOM | 16004 | HD2 | ARG | A1293 | 142.852 | 166.850 | 159.278 | 1.00 | 4.79 | H |
| ATOM | 16005 | HD3 | ARG | A1293 | 142.105 | 167.018 | 160.671 | 1.00 | 4.79 | H |
| ATOM | 16006 | HE | ARG | A1293 | 143.422 | 169.073 | 160.724 | 1.00 | 4.79 | H |
| ATOM | 16007 | HH11 | ARG | A1293 | 141.335 | 168.013 | 158.409 | 1.00 | 4.79 | H |
| ATOM | 16008 | HH12 | ARG | A1293 | 141.014 | 169.356 | 157.857 | 1.00 | 4.79 | H |
| ATOM | 16009 | HH21 | ARG | A1293 | 143.099 | 171.094 | 159.728 | 1.00 | 4.79 | H |
| ATOM | 16010 | HH22 | ARG | A1293 | 142.077 | 171.217 | 158.651 | 1.00 | 4.79 | H |
| ATOM | 16011 | N | LYS | A1294 | 147.501 | 166.198 | 158.981 | 1.00 | 4.64 | N |
| ATOM | 16012 | CA | LYS | A1294 | 147.821 | 165.277 | 157.895 | 1.00 | 4.64 | C |
| ATOM | 16013 | C | LYS | A1294 | 148.866 | 164.252 | 158.324 | 1.00 | 4.64 | C |
| ATOM | 16014 | O | LYS | A1294 | 148.860 | 163.112 | 157.846 | 1.00 | 4.64 | O |
| ATOM | 16015 | CB | LYS | A1294 | 148.306 | 166.054 | 156.672 | 1.00 | 4.64 | C |
| ATOM | 16016 | CG | LYS | A1294 | 147.204 | 166.400 | 155.686 | 1.00 | 4.64 | C |
| ATOM | 16017 | CD | LYS | A1294 | 147.503 | 167.692 | 154.946 | 1.00 | 4.64 | C |
| ATOM | 16018 | CE | LYS | A1294 | 146.398 | 168.037 | 153.962 | 1.00 | 4.64 | C |
| ATOM | 16019 | NZ | LYS | A1294 | 146.613 | 169.365 | 153.327 | 1.00 | 4.64 | N |
| ATOM | 16020 | H | LYS | A1294 | 147.695 | 167.021 | 158.828 | 1.00 | 4.64 | H |
| ATOM | 16021 | HA | LYS | A1294 | 147.017 | 164.795 | 157.644 | 1.00 | 4.64 | H |
| ATOM | 16022 | HB2 | LYS | A1294 | 148.712 | 166.884 | 156.968 | 1.00 | 4.64 | H |
| ATOM | 16023 | HB3 | LYS | A1294 | 148.962 | 165.517 | 156.202 | 1.00 | 4.64 | H |
| ATOM | 16024 | HG2 | LYS | A1294 | 147.123 | 165.688 | 155.033 | 1.00 | 4.64 | H |
| ATOM | 16025 | HG3 | LYS | A1294 | 146.368 | 166.511 | 156.167 | 1.00 | 4.64 | H |
| ATOM | 16026 | HD2 | LYS | A1294 | 147.577 | 168.418 | 155.585 | 1.00 | 4.64 | H |
| ATOM | 16027 | HD3 | LYS | A1294 | 148.331 | 167.594 | 154.451 | 1.00 | 4.64 | H |
| ATOM | 16028 | HE2 | LYS | A1294 | 146.376 | 167.367 | 153.261 | 1.00 | 4.64 | H |
| ATOM | 16029 | HE3 | LYS | A1294 | 145.549 | 168.058 | 154.430 | 1.00 | 4.64 | H |
| ATOM | 16030 | HZ1 | LYS | A1294 | 145.936 | 169.554 | 152.780 | 1.00 | 4.64 | H |
| ATOM | 16031 | HZ2 | LYS | A1294 | 146.663 | 169.996 | 153.953 | 1.00 | 4.64 | H |
| ATOM | 16032 | HZ3 | LYS | A1294 | 147.370 | 169.360 | 152.860 | 1.00 | 4.64 | H |
| HETATM | 19177 | C01 | DRG | A 7 | 131.686 | 178.132 | 193.163 | 1.00 | 7.50 | C |
| HETATM | 19178 | C02 | DRG | A 7 | 131.050 | 176.792 | 195.152 | 1.00 | 7.50 | C |
| HETATM | 19179 | C03 | DRG | A 7 | 130.616 | 175.808 | 192.865 | 1.00 | 7.50 | C |
| HETATM | 19180 | C04 | DRG | A 7 | 129.142 | 176.240 | 192.831 | 1.00 | 7.50 | C |
| HETATM | 19181 | C05 | DRG | A 7 | 128.477 | 175.993 | 191.476 | 1.00 | 7.50 | C |
| HETATM | 19182 | C06 | DRG | A 7 | 128.918 | 174.675 | 190.854 | 1.00 | 7.50 | C |
| HETATM | 19183 | C07 | DRG | A 7 | 130.430 | 174.570 | 190.657 | 1.00 | 7.50 | C |
| HETATM | 19184 | C08 | DRG | A 7 | 131.232 | 175.614 | 191.448 | 1.00 | 7.50 | C |
| HETATM | 19185 | C09 | DRG | A 7 | 133.601 | 175.319 | 190.542 | 1.00 | 7.50 | C |
| HETATM | 19186 | C10 | DRG | A 7 | 133.547 | 176.289 | 189.533 | 1.00 | 7.50 | C |
| HETATM | 19187 | C11 | DRG | A 7 | 134.545 | 176.378 | 188.553 | 1.00 | 7.50 | C |
| HETATM | 19188 | C12 | DRG | A 7 | 135.619 | 175.486 | 188.570 | 1.00 | 7.50 | C |
| HETATM | 19189 | C13 | DRG | A 7 | 135.688 | 174.521 | 189.557 | 1.00 | 7.50 | C |
| HETATM | 19190 | C14 | DRG | A 7 | 134.691 | 174.438 | 190.532 | 1.00 | 7.50 | C |
| HETATM | 19191 | C15 | DRG | A 7 | 135.771 | 173.825 | 185.589 | 1.00 | 7.50 | C |
| HETATM | 19192 | C16 | DRG | A 7 | 134.949 | 172.279 | 184.247 | 1.00 | 7.50 | C |
| HETATM | 19193 | C17 | DRG | A 7 | 135.360 | 171.460 | 185.297 | 1.00 | 7.50 | C |
| HETATM | 19194 | C18 | DRG | A 7 | 135.187 | 170.072 | 185.268 | 1.00 | 7.50 | C |
| HETATM | 19195 | C19 | DRG | A 7 | 134.586 | 169.496 | 184.147 | 1.00 | 7.50 | C |
| HETATM | 19196 | C20 | DRG | A 7 | 133.788 | 167.444 | 183.193 | 1.00 | 7.50 | C |
| HETATM | 19197 | C21 | DRG | A 7 | 134.044 | 165.955 | 183.394 | 1.00 | 7.50 | C |
| HETATM | 19198 | C22 | DRG | A 7 | 133.537 | 165.405 | 184.726 | 1.00 | 7.50 | C |
| HETATM | 19199 | C23 | DRG | A 7 | 133.493 | 163.898 | 184.504 | 1.00 | 7.50 | C |
| HETATM | 19200 | C24 | DRG | A 7 | 133.407 | 163.689 | 182.991 | 1.00 | 7.50 | C |
| HETATM | 19201 | C25 | DRG | A 7 | 133.319 | 165.080 | 182.381 | 1.00 | 7.50 | C |
| HETATM | 19202 | C26 | DRG | A 7 | 134.165 | 170.292 | 183.086 | 1.00 | 7.50 | C |
| HETATM | 19203 | C27 | DRG | A 7 | 134.343 | 171.682 | 183.125 | 1.00 | 7.50 | C |
| HETATM | 19204 | F01 | DRG | A 7 | 136.699 | 173.644 | 189.613 | 1.00 | 7.50 | F |
| HETATM | 19205 | N01 | DRG | A 7 | 131.467 | 176.755 | 193.714 | 1.00 | 7.50 | N |
| HETATM | 19206 | N02 | DRG | A 7 | 132.645 | 175.185 | 191.535 | 1.00 | 7.50 | N |
| HETATM | 19207 | N03 | DRG | A 7 | 136.090 | 175.178 | 185.927 | 1.00 | 7.50 | N |
| HETATM | 19208 | N04 | DRG | A 7 | 135.191 | 173.632 | 184.427 | 1.00 | 7.50 | N |
| HETATM | 19209 | O01 | DRG | A 7 | 137.932 | 174.656 | 187.582 | 1.00 | 7.50 | O |
| HETATM | 19210 | O02 | DRG | A 7 | 137.201 | 177.017 | 187.210 | 1.00 | 7.50 | O |
| HETATM | 19211 | O03 | DRG | A 7 | 134.471 | 168.141 | 184.237 | 1.00 | 7.50 | O |
| HETATM | 19212 | S01 | DRG | A 7 | 136.862 | 175.608 | 187.332 | 1.00 | 7.50 | S |
| HETATM | 19213 | S02 | DRG | A 7 | 136.059 | 172.430 | 186.521 | 1.00 | 7.50 | S |
| HETATM | 19214 | H01 | DRG | A 7 | 132.140 | 178.068 | 192.308 | 1.00 | 7.50 | H |
| HETATM | 19215 | H02 | DRG | A 7 | 132.258 | 178.612 | 193.781 | 1.00 | 7.50 | H |
| HETATM | 19216 | H03 | DRG | A 7 | 130.833 | 178.586 | 193.076 | 1.00 | 7.50 | H |
| HETATM | 19217 | H04 | DRG | A 7 | 130.205 | 177.263 | 195.226 | 1.00 | 7.50 | H |
| HETATM | 19218 | H05 | DRG | A 7 | 130.968 | 175.879 | 195.470 | 1.00 | 7.50 | H |
| HETATM | 19219 | H06 | DRG | A 7 | 131.741 | 177.250 | 195.655 | 1.00 | 7.50 | H |
| HETATM | 19220 | H07 | DRG | A 7 | 130.674 | 174.955 | 193.323 | 1.00 | 7.50 | H |
| HETATM | 19221 | H08 | DRG | A 7 | 128.663 | 175.742 | 193.512 | 1.00 | 7.50 | H |
| HETATM | 19222 | H09 | DRG | A 7 | 129.020 | 177.176 | 193.055 | 1.00 | 7.50 | H |
| HETATM | 19223 | H10 | DRG | A 7 | 127.513 | 176.002 | 191.584 | 1.00 | 7.50 | H |
| HETATM | 19224 | H11 | DRG | A 7 | 128.694 | 176.719 | 190.871 | 1.00 | 7.50 | H |
| HETATM | 19225 | H12 | DRG | A 7 | 128.469 | 174.540 | 190.004 | 1.00 | 7.50 | H |
| HETATM | 19226 | H13 | DRG | A 7 | 128.628 | 173.952 | 191.430 | 1.00 | 7.50 | H |
| HETATM | 19227 | H14 | DRG | A 7 | 130.710 | 173.671 | 190.891 | 1.00 | 7.50 | H |
| HETATM | 19228 | H15 | DRG | A 7 | 130.636 | 174.666 | 189.714 | 1.00 | 7.50 | H |
| HETATM | 19229 | H16 | DRG | A 7 | 131.166 | 176.464 | 190.985 | 1.00 | 7.50 | H |
| HETATM | 19230 | H17 | DRG | A 7 | 132.895 | 174.807 | 192.301 | 1.00 | 7.50 | H |
| HETATM | 19231 | H18 | DRG | A 7 | 132.849 | 176.901 | 189.489 | 1.00 | 7.50 | H |
| HETATM | 19232 | H19 | DRG | A 7 | 134.483 | 177.033 | 187.895 | 1.00 | 7.50 | H |
| HETATM | 19233 | H20 | DRG | A 7 | 134.769 | 173.777 | 191.182 | 1.00 | 7.50 | H |
| HETATM | 19234 | H21 | DRG | A 7 | 135.861 | 175.820 | 185.356 | 1.00 | 7.50 | H |
| HETATM | 19235 | H22 | DRG | A 7 | 135.468 | 169.542 | 185.979 | 1.00 | 7.50 | H |
| HETATM | 19236 | H23 | DRG | A 7 | 134.133 | 167.697 | 182.322 | 1.00 | 7.50 | H |
| HETATM | 19237 | H24 | DRG | A 7 | 132.839 | 167.640 | 183.238 | 1.00 | 7.50 | H |
| HETATM | 19238 | H25 | DRG | A 7 | 134.998 | 165.788 | 183.346 | 1.00 | 7.50 | H |
| HETATM | 19239 | H26 | DRG | A 7 | 132.645 | 165.735 | 184.918 | 1.00 | 7.50 | H |
| HETATM | 19240 | H27 | DRG | A 7 | 134.109 | 165.644 | 185.471 | 1.00 | 7.50 | H |
| HETATM | 19241 | H28 | DRG | A 7 | 134.295 | 163.478 | 184.850 | 1.00 | 7.50 | H |
| HETATM | 19242 | H29 | DRG | A 7 | 132.737 | 163.498 | 184.959 | 1.00 | 7.50 | H |
| HETATM | 19243 | H30 | DRG | A 7 | 132.645 | 163.141 | 182.744 | 1.00 | 7.50 | H |
| HETATM | 19244 | H31 | DRG | A 7 | 134.204 | 163.234 | 182.679 | 1.00 | 7.50 | H |
| HETATM | 19245 | H32 | DRG | A 7 | 132.390 | 165.343 | 182.297 | 1.00 | 7.50 | H |
| HETATM | 19246 | H33 | DRG | A 7 | 133.722 | 165.117 | 181.499 | 1.00 | 7.50 | H |
| HETATM | 19247 | H34 | DRG | A 7 | 133.761 | 169.928 | 182.332 | 1.00 | 7.50 | H |
| HETATM | 19248 | H35 | DRG | A 7 | 134.062 | 172.211 | 182.414 | 1.00 | 7.50 | H |
| HETATM | 19249 | H36 | DRG | A 7 | 132.274 | 176.379 | 193.694 | 1.00 | 7.50 | H |
| TER | | | | | | | | | | |
| END | | | | | | | | | | |

We claim:

1. A compound of Formula I; 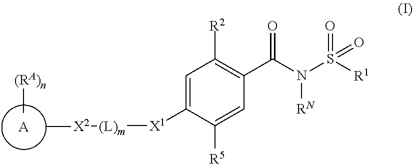
and pharmaceutically acceptable salts thereof, wherein;
R¹ is selected from a first set of moieties consisting of C₁₋₈alkyl, C₃₋₁₂cycloalkyl, C-linked C₂₋₁₁heterocycloalkyl, C₃₋₁₂carbocycle, aryl, heteroaryl, and —NR^{1A}R^{1B}, wherein;
R^{1A} and R^{1B} are selected from H, C₁₋₈alkyl, C₃₋₈cycloalkyl, C₁₋₈haloalkyl, or R^{1A} and R^{1B} together with the nitrogen atom to which they are both bonded form a 3 to 10 membered heterocyclic group;
any of the first set of moieties, R^{1A}, or R^{1B}, is optionally substituted with one or more substituents selected from a second set of moieties consisting of:
C₁₋₈alkyl, C₂₋₈alkenyl, C₃₋₈cycloalkyl, C₁₋₈haloalkyl, aryl, (aryl)C₁₋₈alkyl, heteroaryl, (heteroaryl)C₁₋₈alkyl, C₂₋₁₁heterocycloalkyl, (C₂₋₁₁heterocycloalkyl)C₁₋₈alkyl, F, Cl, Br, I, —OH, —CN, —NO₂, —O,
aminocarbonyl, —X^{R1}NR^{R1a}R^{R1b}, —X^{R1}OR^{R1a}, and —X^{R1}SR^{R1a}, wherein;
X^{R1} is selected from the group consisting of C(═O), C₁₋₄ alkylene, C₁₋₄heteroalkylene, C₂₋₄alkenylene and C₂₋₄ alkynylene, or is absent; and
R^{R1a} and R^{R1b} are independently selected from a third set of moieties consisting of: H, C₁₋₈alkyl, C₂₋₈alkenyl, C₁₋₈haloalkyl, C₃₋₈cycloalkyl, C₃₋₁₂carbocycle, (C₃₋₈ cycloalkyl)C₁₋₈ alkyl, aryl, (aryl)C₁₋₈alkyl, heteroaryl, (heteroaryl)C₁₋₈alkyl, C₂₋₁₁heterocycloalkyl, and (C₂₋₁₁heterocycloalkyl)C₁₋₈alkyl,
or R^{R1a} and R^{R1b} together with the nitrogen atom to which they are both bonded form a 3 to 10 membered heterocyclic group; and
wherein any of the second set of moieties, R^{R1a}, or R^{R1b}, where present, are each optionally substituted by one or more groups independently selected from: C₁₋₈ alkyl, C₁₋₈haloalkyl, F, Cl, Br, I, —OH, —CN, aryl, C₁₋₈haloalkyl-substituted aryl, C₁₋₈alkoxy, C₁₋₈alkanoyl, C₁₋₈alkoxycarbonyl, C₃₋₈cycloalkyl, C₂₋₁₁heterocycloalkyl, amino, (C₁₋₃alkyl)amino, di(C₁₋₃alkyl)-amino, C₁₋₃alkylamido, C₁₋₃alkylcarboxy, and —NO₂;
R^{N} is hydrogen, C₁₋₄ alkyl, or C₁₋₄haloalkyl;
R² is selected from the group consisting of H, F, Cl, Br, I, —CN, C₁₋₈ alkyl, C₁₋₈ haloalkyl and C₁₋₈ alkoxy;
R⁵ is selected from the group consisting of H, F, Cl, Br, I, —CN, C₁₋₈ alkyl, C₂₋₈ alkenyl, C₂₋₈ alkynyl, C₁₋₈ haloalkyl, C₁₋₈ alkoxy, C₃₋₈ cycloalkyl, C₂₋₁₁ heterocycloalkyl, phenyl and 5-6 membered heteroaryl comprising 1 to 3 heteroatoms selected from N, O and S,
wherein said 5-6 membered heteroaryl, C₁₋₈ alkyl, C₃₋₈ cycloalkyl or C₂₋₁₁ heterocycloalkyl is further optionally substituted with from 1 to 3 substituents selected from F, Cl, Br, I, —OH, —O, C₃₋₆ cycloalkyl, —CN, C₁₋₄ alkyl, —C₁₋₄ alkyl-O—C₁₋₄ alkyl, C₁₋₄ haloalkyl and C₁₋₄ alkoxy;
L is a linker selected from the group consisting of C₁₋₄ alkylene, C₂₋₄ alkenylene, C₂₋₄ alkynylene, and C₁₋₄ heteroalkylene, wherein L is optionally substituted with from 1 to 3 substituents selected from the group consisting of ═O, —OH, —OCH₂-phenyl, C₁₋₄ alkyl, C₁₋₄ haloalkyl and C₁₋₄ acyl;
m is 0 or 1;
X¹ and X² are each independently selected from the group consisting of, —O—, —S(O)—, —S(O)₂— and —N(R^{N})—, wherein R^{x} is H, C₁₋₈ alkyl, C₁₋₈ acyl and —S(O)₂(C₁₋₈ alkyl), or is absent;
and wherein if m is 0 then at least one of X¹ or X² is absent;
n is an integer from 0 to 5;
A is selected from the group consisting of: hydrogen, C₁₋₈alkyl, C₁₋₈haloalkyl, C₃₋₁₂ cycloalkyl, and aryl, and wherein if A is hydrogen then n is 0; and
each R^{A} is independently selected from the group consisting of C₁₋₈ alkyl, C₃₋₈ cycloalkyl, C₁₋₈ haloalkyl, F, Cl, Br, I, —OH, —CN, —NO₂, and ═O.

2. A compound of Formula II; 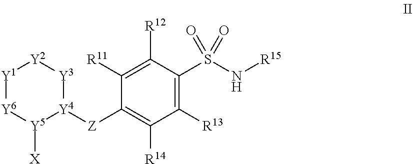
or a pharmaceutically acceptable salt thereof, wherein;
R¹¹, R¹², R¹³, and R¹⁴ are each independently selected from hydrogen, C₁-C₆ alkyl, cyano, C₃-C₆ cycloalkyl, hydroxy, C₁-C₆ alkoxy, —NH₂, —NHR, —NR₂, —SR, —S(O)R, —SO₂R, SO₂NR₂, nitro, and halo, wherein any C₁-C₆ alkyl, and C₃-C₆ cycloalkyl is optionally substituted with one or more groups independently selected from halo, cyano, hydroxy,
C₁-C₆ alkoxy, C₁-C₆ haloalkoxy, C₃-C₆ cycloalkyl, and phenyl;
Z is selected from —N(R¹⁵)—, —O—, —S—, —S(O)—, and —S(O)₂—;
R¹⁵ is pyridyl that is substituted with —(X^{15R})₀₋₁OR^{R15a} or —(X^{15R})₀₋₁SR^{R15a}, wherein X^{15R} is C₁₋₄ alkylene; wherein R^{R15a} is hydrogen, C₁₋₈ alkyl, C₂₋₈ alkenyl, C₁₋₈ haloalkyl, C₃₋₈ cycloalkyl, (C₃₋₈ cycloalkyl)C₁₋₈ alkyl, phenyl, (aryl)C₁₋₈ alkyl, and C₂₋₁₁ heterocycloalkyl;
where any C₂₋₈ alkenyl, C₃₋₈ cycloalkyl, (C₃₋₈ cycloalkyl)C₁₋₈ alkyl, phenyl, (aryl)C₁₋₈ alkyl, and C₂₋₁₁ heterocycloalkyl of R^{R15a} is optionally substituted with from 1 to 5 substituents independently selected from the group consisting of C₁₋₈ haloalkyl, F, Cl, Br, I, —OH, —CN, and —NO₂;
X is selected from hydrogen, C₁₋₈alkyl, —N(R¹⁶)₂ and —N(R¹⁷)₃^{+ -}W, and amine oxides thereof;
Y¹ is —C(R¹⁸)₂—; and Y² is selected from —(C(R¹⁸)₂)ₙ—, —N(R¹⁹)—, —O—, —C(R¹⁸)₂—N(R¹⁹)—, —N(R¹⁹)—C(R¹⁸)₂—, —C(R¹⁸)₂—O—, and —O—C(R¹⁸)₂—; or
Y¹ is selected from —N(R¹⁹)— and —O—; and Y² is —(C(R¹⁸)₂)ₙ—;
n is selected from 0, 1, and 2;
Y³ and Y⁶ are each —C(R¹⁸)₂—;
Y⁴ and Y⁵ are each —C(R¹⁸)—;
each R¹⁶ is independently selected from hydrogen, C₁-C₆ alkyl, C₃-C₆ cycloalkyl, C₁-C₆ alkanoyl, 4-7 membered heterocycle, 5-6 membered heteroaryl, and C₆-C₁₂ aryl, wherein any C₁-C₆ alkyl, C₃-C₆ cycloalkyl, C₁-C₆ alkanoyl, 4-7 membered heterocycle, 5-6 membered heteroaryl, and C₆-C₁₂ aryl is optionally substituted with one or more groups independently selected from deuterium, halo, cyano, hydroxy, C₁-C₆ alkyl, C₁-C₆ alkoxy, C₁-C₆ alkanoyl, C₁-C₆ haloalkyl, C₁-C₆ haloalkoxy, C₃-C₆ cycloalkyl, C₃-C₆ halocycloalkyl, —NR^{a}R^{b}, —C(═O)NR^{a}R^{b}, Re, and phenyl, wherein any C₁-C₆ alkyl, C₁-C₆ alkoxy, and C₁-C₆ alkanoyl is optionally substituted with C₃-C₆ cycloalkyl; or two R¹⁶ groups together with the nitrogen to which they are both attached form a 4- to 10-membered heterocycle that is optionally substituted with one or more groups independently selected from deuterium, halo, cyano, hydroxy, C₁-C₆ alkyl, and C₃-C₆ cycloalkyl, which C₁-C₆ alkyl, and C₃-C₆ cycloalkyl is optionally substituted with one or more groups independently selected from hydroxy and halo;
each R¹⁷ is independently selected from C₁-C₆ alkyl; or two R¹⁷ groups together with the nitrogen to which they are both attached form a 4- to 7-membered heterocycle;
each R¹⁸ is independently selected from hydrogen, C₁-C₆ alkyl, C₁-C₆ haloalkyl, C₃-C₆ cycloalkyl, benzyl, 5-15 membered heteroaryl, C₁-C₆ alkoxy, C₁-C₆ haloalkoxy, C₃-C₆ cycloalkoxy, hydroxy, halo, cyano, and -L-C₆-C₁₂ aryl; wherein each C₃-C₆ cycloalkyl, -L-C₆-C₁₂ aryl, benzyl, 5-15 membered heteroaryl, and C₁-C₆ alkoxy, is optionally substituted with one to three substituents R^{x} each independently selected from C₁-C₆ alkyl, C₃-C₆ cycloalkyl, C₁-C₆ alkoxy, C₁-C₆ haloalkoxy, cyano, halo, hydroxy, C₃-C₆ cycloalkoxy, C₃-C₆ halocycloalkyl, —S(C₁-C₆ alkyl), —S(O)(C₁-C₆ alkyl), —S(O)₂(C₁-C₆ alkyl), —NH₂ —NH(C₁-C₆ alkyl), —N(C₁-C₆ alkyl)₂, 4-6 membered heterocycle, and C₁-C₆ haloalkyl or two R¹⁸ that are on adjacent carbons taken together form a double bond; and
R¹⁹ is selected from hydrogen, C₁-C₆ alkyl and C₆-C₁₂ aryl, which C₆-C₁₂ aryl is optionally substituted with one to three substituents each independently selected from C₁-C₆ alkyl, C₃-C₆ cycloalkyl, C₁-C₆ alkoxy, C₁-C₆ haloalkoxy, cyano, halo, and C₁-C₆ haloalkyl; or
one R¹⁸ or R¹⁹ taken together with another R¹⁸ or R¹⁹ and the atoms to which they are attached form a 3-8 membered fused, bridged or spirocyclic ring, which 3-8 membered ring is optionally substituted with one to three substituents each independently selected from C₁-C₆ alkyl, C₁-C₆ alkoxy, cyano, halo, and C₁-C₆ haloalkyl; and L is selected from a bond, —O—, —S—, —S(O)—, and —S(O)₂—;
each R^{a} and R^{b} is independently selected from the group consisting of hydrogen, C₁-C₆ alkyl, and C₁-C₆ alkanoyl;
each R^{c} is independently selected from 4-7 membered heterocycle that is optionally substituted with one or more groups independently selected from halo, C₁-C₆ alkyl, and
C₁-C₆ haloalkyl; and
W⁻ is a counterion.

3. A compound of Formula III; 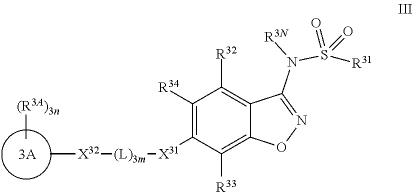
or a pharmaceutically acceptable salt thereof, wherein;
R³¹ is selected from a first set of moieties consisting of C₁₋₈alkyl, C₃₋₁₂cycloalkyl, C-linked C₂₋₁₁heterocycloalkyl, C₃₋₁₂carbocycle, aryl, heteroaryl, and —NR^{31A}R^{31B}, wherein;
R^{31A} and R^{31B} are selected from H, C₁₋₈alkyl, C₃₋₈cycloalkyl, C₁₋₈haloalkyl, or R^{31A} and R^{31B} together with the nitrogen atom to which they are both bonded form a 3 to 10 membered heterocyclic group;
any of the first set of moieties, R^{31A}, or R^{31B}, is optionally substituted with one or more substituents selected from a second set of moieties consisting of:
C₁₋₈alkyl, C₂₋₈alkenyl, C₃₋₈cycloalkyl, C₁₋₈haloalkyl, aryl, (aryl)C₁₋₈alkyl, heteroaryl, (heteroaryl)C₁₋₈alkyl, C₂₋₁₁heterocycloalkyl,
(C₂₋₁₁heterocycloalkyl)C₁₋₈alkyl, F, Cl, Br, I, —OH, —CN, —NO₂, ═O, aminocarbonyl, —X^{R1}NR^{R1a}R^{R1b},
—X^{R1}OR^{R1a}, and —X^{R1}SR^{R1a}, wherein;
X^{R1} is selected from the group consisting of C(═O), C₁₋₄ alkylene, C₁₋₄heteroalkylene, C₂₋₄alkenylene and C₂₋₄ alkynylene, or is absent; and
R^{R1a} and R^{R1b} are independently selected from a third set of moieties consisting of: H, C₁₋₈alkyl, C₂₋₈alkenyl, C₁₋₈haloalkyl, C₃₋₈cycloalkyl, C₃₋₁₂carbocycle, (C₃₋₈ cycloalkyl)C₁₋₈ alkyl, aryl, (aryl)C₁₋₈alkyl, heteroaryl, (heteroaryl)C₁₋₈alkyl, C₂₋₁₁heterocycloalkyl, and
(C₂₋₁₁heterocycloalkyl)C₁₋₈alkyl,
or R^{R1a} and R^{R1b} together with the nitrogen atom to which they are both bonded form a 3 to 10 membered heterocyclic group;
and
wherein any of the second set of moieties, R^{R1a}, or R^{R1b}, where present, are each optionally substituted by one or more groups independently selected from: C₁₋₈ alkyl, C₁₋₈haloalkyl, F, Cl, Br, I, —OH, —CN, aryl, C₁₋₈haloalkyl-substituted aryl, C₁₋₈alkoxy, C₁₋₈alkanoyl, C₁₋₈alkoxycarbonyl, C₃₋₈cycloalkyl, C₂₋₁₁heterocycloalkyl, amino, (C₁₋₃alkyl)amino, di(C₁₋₃alkyl)amino, C₁₋₃alkylamido, C₁₋₃alkylcarboxy, and —NO₂;
R^{3N} is hydrogen, C₁₋₄ alkyl or C₁₋₄ haloalkyl;
R³² and R³³ are independently selected from the group consisting of H, F, Cl, Br, I, —CN, C₁₋₈ alkyl, C₁₋₈ haloalkyl and C₁₋₈ alkoxy;
R³⁴ is selected from the group consisting of H, F, Cl, Br, I, —CN, C₁₋₈ alkyl, C₂₋₈ alkenyl, C₁₋₈haloalkyl, C₁₋₈ alkoxy, C₃₋₈ cycloalkyl, C₂₋₁₁ heterocycloalkyl, phenyl and 5-6 membered heteroaryl comprising 1 to 3 heteroatoms selected from N, O and S, wherein said 5-6 membered heteroaryl, C₁₋₈ alkyl, C₃₋₈ cycloalkyl or C₂₋₁₁ heterocycloalkyl is further optionally substituted with from 1 to 3 R^{5a} substituents selected from F, Cl, Br, I, —OH, ═O, C₃₋₆ cycloalkyl, —CN, C₁₋₄ alkyl, —C₁₋₄ alkyl-O—C₁₋₄ alkyl, C₁₋₄ haloalkyl and C₁₋₄ alkoxy;
L is a linker selected from the group consisting of C₁₋₄ alkylene, C₂₋₄ alkenylene, C₂₋₄ alkynylene, and C₁₋₄ heteroalkylene, wherein L is optionally substituted with from 1 to 3
R^{L} substituents selected from the group consisting of ═O, —OH, —OCH₂-phenyl, C₁₋₄ alkyl, C₁₋₄ haloalkyl and C₁₋₄ acyl;
3m is 0 or 1;
X³¹ and X³² are each independently selected from the group consisting of absent, —O—, —S(O)—, —S(O)₂— and —N(R^{N})— wherein R^{x} is H, C₁₋₈ alkyl, C₁₋₈ acyl or —S(O)₂(C₁₋₈ alkyl), and
wherein if 3m is 0 then at least one of X¹ or X² is absent;
3n is an integer from 0 to 5;
3A is selected from the group consisting of hydrogen, C₁₋₈alkyl, C₁₋₈haloalkyl, C₃₋₁₂ cycloalkyl, and aryl, and wherein if 3A is hydrogen then n is 0; and
each R^{3A} is independently selected from the group consisting of C₁₋₈ alkyl, C₃₋₈ cycloalkyl, C₁₋₈ haloalkyl, F, Cl, Br, I, —OH, —CN, —NO₂, and ═O.

4. A pharmaceutical composition comprising a compound or a pharmaceutically acceptable salt as described in claim 1 and a pharmaceutically acceptable excipient.

5. A method of treating a disease or condition in a mammal selected from the group consisting of pain, depression, cardiovascular diseases, respiratory diseases, and psychiatric diseases, and combinations thereof, wherein the method comprises administering to the mammal in need thereof a therapeutically effective amount of a compound or a pharmaceutically acceptable salt as described in claim 1.

6. The method of claim 27, wherein said disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, persistent pain, peripherally mediated pain, centrally mediated pain, chronic headache, migraine headache, sinus headache, tension headache, phantom limb pain, dental pain, peripheral nerve injury or a combination thereof.

7. The method of claim 27, wherein said disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritis, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxi related illnesses, familial erythromelalgia, primary erythromelalgia, familial rectal pain, cancer, epilepsy, partial and general tonic seizures, restless leg syndrome, arrhythmias, fibromyalgia, neuroprotection under ischaemic conditions cause by stroke or neural trauma, tach-arrhythmias, atrial fibrillation and ventricular fibrillation.

8. A method of treating pruritus in a mammal, wherein the method comprises administering to the mammal in need thereof a therapeutically effective amount of a compound or a pharmaceutically acceptable salt as described in claim 1.

9. A computer-based method of designing an inhibitor of NaV1.7, the method comprising docking molecular structures of compounds into a model of the NaV1.7 binding site based on coordinates found in the PDB File in Appendix 1.

10. A method of identifying a compound that binds to the VSD4 domain of the NaV1.7 receptor, the method comprising:
computationally modeling a test molecule that fits spatially into an atomic structural model of the NaV1.7 receptor VSD4 binding site or portion thereof, wherein said atomic structural model comprises atomic coordinates found in Appendix 1; and
screening said test molecule in an assay characterized by binding of the test molecule to the VSD4 binding site of the NaV1.7 receptor, thereby identifying a compound that inhibits NaV1.7 receptor activity.
